(19) United States
(12) Patent Application Publication
(10) Pub. No.: US 2026/0151470 A1
(43) Pub. Date: Jun. 4, 2026

(54) COMPOSITIONS FOR DELIVERY OF PLASMODIUM CSP ANTIGENS AND RELATED METHODS

(71) Applicant: BioNTech SE, Mainz (DE)

(72) Inventors: Ugur Sahin, Mainz (DE); Asaf Poran, Stoneham, MA (US); Daniel Abram Rothenberg, Melrose, MA (US); Stephanie Erbar, Chiclana de la Frontera (ES); Annette Vogel, Mainz (DE); Patricia dos Santos Meireles, Mainz (DE); John Srouji, Danville, CA (US); Anja Dokic, Mainz (DE); Thorsten Klamp, Mainz (DE); Charles Jennison, Mainz (DE)

(21) Appl. No.: 19/114,305

(22) PCT Filed: Sep. 22, 2023
(86) PCT No.: PCT/US23/74959

§ 371 Date: Mar. 21, 2025

Related U.S. Application Data
(60) Provisional application No. 63/486,619, filed on Feb. 23, 2023.
(60) Provisional application No. 63/515,329, filed on Jul. 24, 2023.

(30) Foreign Application Priority Data
Sep. 23, 2022 (WO) PCT/US22/44626

(51) Int. Cl. A61K 39/015; A61K 39/00; A61P 37/04; C07K 14/445

(52) U.S. Cl. CPC A61K 39/015; A61P 37/04; C07K 14/445; A61K 2039/53; A61K 2039/6093

(57) ABSTRACT

The present disclosure provides compositions (e.g., pharmaceutical compositions) for delivery of malarial protein antigens and related technologies (e.g., components thereof and/or methods relating thereto). Among other things, the present disclosure provides polyribonucleotides encoding malarial protein antigens.

### BACKGROUND

Malaria is a mosquito-borne infectious disease caused by protozoan parasites of the Plasmodium genus. According to the World Health Organization, an estimated 3.4 billion people in 92 countries are at risk of being infected with the Plasmodium parasite and developing disease.

### SUMMARY

The present disclosure provides technologies (e.g., compositions, methods, etc.) for delivery of Plasmodium antigens (also referred to herein as “malaria antigens” or “malarial antigens”). In one aspect, provided herein is a polyribonucleotide encoding a polypeptide, wherein the polypeptide comprises one or more Plasmodium CSP polypeptide regions or portions thereof. In some embodiments, each of the one or more Plasmodium CSP polypeptide regions or portions thereof comprise 25 or more contiguous amino acids of the amino acid sequence according to SEQ ID NO: 1. In some embodiments, a “fragment” of a polypeptide is a “portion” of a polypeptide.

In some embodiments, the polypeptide encoded by a provided polyribonucleotide comprises one or more repeats of the amino acid sequence of NANPNVDP (SEQ ID NO: 102), and the polypeptide does not comprise the amino acid sequence of NPNA (SEQ ID NO: 141). In some embodiments, the polypeptide encoded by a provided polyribonucleotide comprises five or more repeats of the amino acid sequence of NANPNVDP (SEQ ID NO: 102).

One aspect provided herein relates to a polyribonucleotide encoding a polypeptide, wherein the polypeptide comprises: (i) a heterologous secretory signal and (ii) one or more Plasmodium CSP polypeptide regions or portions thereof.

One aspect provided herein relates to a polyribonucleotide encoding a polypeptide, wherein the polypeptide comprises: (i) one or more Plasmodium CSP polypeptide regions or portions thereof and (ii) a heterologous transmembrane region.

In some embodiments, a polypeptide encoded by a polyribonucleotide comprises one or more repeats of the amino acid sequence of NANPNVDP (SEQ ID NO: 102). In some embodiments, a polypeptide comprises two or more repeats of the amino acid sequence of NANPNVDP (SEQ ID NO: 102). In some embodiments, a polypeptide comprises between two and twelve repeats of the amino acid sequence of NANPNVDP (SEQ ID NO: 102). In some embodiments, a polypeptide comprises exactly three repeats of the amino acid sequence of NANPNVDP (SEQ ID NO: 102). In some embodiments, a polypeptide comprises between four and twelve repeats of the amino acid sequence of NANPNVDP (SEQ ID NO: 102). In some embodiments, a polypeptide comprises: (i) exactly eight repeats of the amino acid sequence of NANPNVDP (SEQ ID NO: 102); or (ii) exactly nine repeats of the amino acid sequence of NANPNVDP (SEQ ID NO: 102). In some embodiments, repeats of the amino acid sequence of NANPNVDP (SEQ ID NO: 102) are all contiguous with each other. In some embodiments, repeats of the amino acid sequence of NANPNVDP (SEQ ID NO: 102) are not all contiguous with each other.

In some embodiments, a polypeptide encoded by a polyribonucleotide comprises four portions of a Plasmodium CSP polypeptide, and each portion comprises two contiguous repeats of the amino acid sequence of NANPNVDP (SEQ ID NO: 102).

In some embodiments, a polypeptide encoded by a polyribonucleotide comprises one or more Plasmodium CSP C-terminal regions or portions thereof. In some embodiments, a polypeptide comprises exactly one Plasmodium CSP C-terminal region, and the Plasmodium CSP C-terminal region comprises or consists of an amino acid sequence with at least 85%, at least 90%, at least 95%, at least 96%, at least 97%, at least 98%, at least 99%, or 100% sequence identity to an amino acid sequence according to SEQ ID NO: 1. In some embodiments, a polypeptide comprises two or more portions of a Plasmodium CSP C-terminal region. In some embodiments, a polypeptide comprises one or more portions of the Plasmodium CSP C-terminal region, wherein each of the one or more portions comprise or consist of: (i) an amino acid sequence according to SEQ ID NO: 111, (ii) an amino acid sequence according to SEQ ID NO: 114, (iii) an amino acid sequence according to SEQ ID NO: 117, (iv) an amino acid sequence according to SEQ ID NO: 120, or (v) a combination thereof. In some embodiments, a polypeptide comprises one portion of the Plasmodium CSP C-terminal region, wherein the portion comprises or consists of: (i) an amino acid sequence according to SEQ ID NO: 111, (ii) an amino acid sequence according to SEQ ID NO: 114, (iii) an amino acid sequence according to SEQ ID NO: 117, (iv) an amino acid sequence according to SEQ ID NO: 120, or (v) a combination thereof. In some embodiments, a polypeptide comprises one or more portions of the Plasmodium CSP C-terminal region, wherein the one or more portions collectively comprise or consist of: (i) an amino acid sequence according to SEQ ID NO: 111, (ii) an amino acid sequence according to SEQ ID NO: 114, (iii) an amino acid sequence according to SEQ ID NO: 117, and (iv) an amino acid sequence according to SEQ ID NO: 120.

In some embodiments, a polypeptide encoded by a provided polyribonucleotide comprises a serine immediately following the Plasmodium CSP C-terminal region. In some embodiments, a polypeptide comprises a serine-valine sequence immediately following the Plasmodium CSP C-terminal region.

In some embodiments, a polypeptide encoded by a provided polyribonucleotide comprises one or more Plasmodium CSP junction regions or portions thereof. In some embodiments, a polypeptide comprises two or more Plasmodium CSP junction regions or portions thereof. In some embodiments, two or more Plasmodium CSP junction regions consist of an amino acid sequence according to SEQ ID NO: 126. In some embodiments, a polypeptide comprises two or more portions of a Plasmodium CSP junction region. In some embodiments, two or more portions of a Plasmodium CSP junction region comprise a deletion of one or more of K93, L94, K95, Q96 and P97, wherein the amino acid numbering is relative to SEQ ID NO: 1. In some embodiments, two or more portions of a Plasmodium CSP junction region comprise a deletion of K93, L94, K95, and Q96, wherein the amino acid numbering is relative to SEQ ID NO: 1. In some embodiments, two or more portions of a Plasmodium CSP junction region comprise a deletion of K93, L94, K95, Q96 and P97, wherein the amino acid numbering is relative to SEQ ID NO: 1.

In some embodiments, a polypeptide encoded by a provided polyribonucleotide comprises exactly one Plasmodium CSP junction region. In some embodiments, a Plasmodium CSP junction region consists of an amino acid sequence according to SEQ ID NO: 126. In some embodiments, a polypeptide comprises one or more portions of a Plasmodium CSP junction region. In some embodiments, one or more portions of a Plasmodium CSP junction region comprise a deletion of one or more of K93, L94, K95, Q96 and P97, wherein the amino acid numbering is relative to SEQ ID NO: 1. In some embodiments, one or more portions of a Plasmodium CSP junction region comprise a deletion of K93, L94, K95, and Q96, wherein the amino acid numbering is relative to SEQ ID NO: 1. In some embodiments, one or more portions of a Plasmodium CSP junction region comprise a deletion of K93, L94, K95, Q96 and P97, wherein the amino acid numbering is relative to SEQ ID NO: 1. In some embodiments, each portion of a Plasmodium CSP junction region comprises or consists of an amino acid sequence according to SEQ ID NO: 129. In some embodiments, each portion of a Plasmodium CSP junction region comprises or consists of an amino acid sequence according to SEQ ID NO: 132. In some embodiments, each portion of a Plasmodium CSP junction region comprises or consists of an amino acid sequence according to SEQ ID NO: 129.

In some embodiments, a polypeptide encoded by a provided polyribonucleotide comprises one or more Plasmodium CSP junction region variants. In some embodiments, a Plasmodium CSP junction region variant comprises one or more substitution mutations. In some embodiments, one or more substitution mutations comprise a K93A mutation, an L94A mutation, or both, wherein the amino acid numbering is relative to SEQ ID NO: 1. In some embodiments, each Plasmodium CSP junction region variant comprises the amino acid sequence of AAKQ (SEQ ID NO: 426).

In some embodiments, a polypeptide encoded by a provided polyribonucleotide comprises one or more Plasmodium CSP N-terminal end regions or portions thereof. In some embodiments, a polypeptide comprises two or more Plasmodium CSP N-terminal end regions or portions thereof. In some embodiments, each Plasmodium CSP N-terminal end region consists of an amino acid sequence according to SEQ ID NO: 135.

In some embodiments, a polypeptide encoded by a provided polyribonucleotide does not comprise a Plasmodium CSP N-terminal end region or any portion thereof. In some embodiments, a polypeptide comprises one or more Plasmodium CSP N-terminal regions or portions thereof. In some embodiments, a polypeptide comprises two or more Plasmodium CSP N-terminal regions or portions thereof. In some embodiments, each Plasmodium CSP N-terminal region comprises or consists of an amino acid sequence with at least 85%, at least 90%, at least 95%, at least 96%, at least 97%, at least 98%, at least 99%, or 100% sequence identity to an amino acid sequence according to SEQ ID NO: 138.

In some embodiments, a polypeptide encoded by a provided polyribonucleotide does not comprise a Plasmodium CSP N-terminal region or any portion thereof.

In some embodiments, a polypeptide encoded by a provided polyribonucleotide comprises one or more Plasmodium CSP major repeat regions or portions thereof. In some embodiments, one or more Plasmodium CSP major repeat regions or portions thereof comprise the amino acid sequence NANPNA (SEQ ID NO: 153) or NPNANP (SEQ ID NO: 150). In some embodiments, a polypeptide comprises exactly one Plasmodium CSP major repeat region or portion thereof, and the Plasmodium CSP major repeat region or portion thereof comprises a total of at least 2 and at most 35 repeats of the amino acid sequence NANP (SEQ ID NO: 147). In some embodiments, a Plasmodium CSP major repeat region or portion thereof comprises two contiguous stretches of repeats of the amino acid sequence NANP (SEQ ID NO: 147), and wherein the two contiguous stretches of repeats of the amino acid sequence NANP (SEQ ID NO: 147) flank an amino acid sequence of NVDP (SEQ ID NO: 144). In some embodiments, a Plasmodium CSP major repeat region comprises, in N-terminus to C-terminus order, 17 repeats of the amino acid sequence NANP (SEQ ID NO: 147), an amino acid sequence of NVDP (SEQ ID NO: 144), and 18 repeats of the amino acid sequence NANP (SEQ ID NO: 147). In some embodiments, a portion of the Plasmodium CSP major repeat region consists of at most 18 contiguous repeats of the amino acid sequence NANP (SEQ ID NO: 147). In some embodiments, a portion of the Plasmodium CSP major repeat region consists of 2 contiguous repeats of the amino acid sequence NANP (SEQ ID NO: 147). In some embodiments, a Plasmodium CSP major repeat region comprises or consists of an amino acid sequence with at least 85%, at least 90%, at least 95%, at least 96%, at least 97%, at least 98%, at least 99%, or 100% sequence identity to an amino acid sequence according to SEQ ID NO: 156.

In some embodiments, a polypeptide encoded by a provided polyribonucleotide does not comprise a Plasmodium CSP major repeat region or a portion of a Plasmodium CSP major repeat region comprising the amino acid sequence NPNA (SEQ ID NO: 141).

In some embodiments, one or more Plasmodium CSP polypeptide regions or portions thereof, if present in the polypeptide encoded by a provided polyribonucleotide, are in the following N-terminus to C-terminus order: (i) one or more Plasmodium CSP N-terminal regions or portions thereof, (ii) one or more Plasmodium CSP N-terminal end regions or portions thereof, (iii) one or more Plasmodium CSP junction regions, portions thereof, or variants thereof, (iv) one or more repeats of the amino acid sequence of NANPNVDP (SEQ ID NO: 102), (v) one or more Plasmodium CSP major repeat regions or portions thereof, and (vi) one or more Plasmodium CSP C-terminal regions or portions thereof.

In some embodiments, one or more Plasmodium CSP polypeptide regions or portions thereof, if present in the polypeptide encoded by a provided polyribonucleotide, are in the following N-terminus to C-terminus order: (i) one Plasmodium CSP N-terminal region or portion thereof, (ii) one Plasmodium CSP N-terminal end region or portion thereof, (iii) one Plasmodium CSP junction region, portion thereof, or variant thereof, (iv) one or more repeats of the amino acid sequence of NANPNVDP (SEQ ID NO: 102), (v) one Plasmodium CSP major repeat region or portion thereof, and (vi) one Plasmodium CSP C-terminal region or portion thereof.

In some embodiments, a polypeptide encoded by a provided polyribonucleotide comprises one or more helper antigens. In some embodiments, one or more helper antigens comprise a Plasmodium antigen. In some embodiments, one or more helper antigens are Plasmodium 2-phospho-D-glycerate hydro-lyase antigen, Plasmodium liver stage antigen 1 (a), (LSA-1 (a)), Plasmodium liver stage antigen 1 (b) (LSA-1 (b)), Plasmodium thrombospondin-related anonymous protein (TRAP), Plasmodium liver stage associated protein 1 (LSAP1), Plasmodium liver stage associated protein 2 (LSAP2), Plasmodium UIS3, Plasmodium UIS4, Plasmodium ETRAP10.3, Plasmodium liver specific protein 1 (LISP-1), Plasmodium liver specific protein 2 (LISP-2), Plasmodium liver stage antigen 3 (LSA-3), Plasmodium EXP1, Plasmodium E140, Plasmodium reticulocyte-binding protein homolog 5 (Rh5), Plasmodium glutamic acid-rich protein (GARP), Plasmodium parasite-infected erythrocyte surface protein 2 (PIESP2), Plasmodium Cysteine-Rich Protective Antigen (CyRPA), Plasmodium Ripr, Plasmodium P113, or a combination thereof. In some embodiments, one or more helper antigens comprise or consist of a P. falciparum 2-phospho-D-glycerate hydro-lyase antigen. In some embodiments, a P. falciparum 2-phospho-D-glycerate hydro-lyase antigen comprises or consists of an amino acid sequence according to SEQ ID NO: 240. In some embodiments, one or more helper antigens comprise or consist of a P. falciparum liver-stage antigen 3. In some embodiments, a P. falciparum liver-stage antigen 3 comprises or consists of an amino acid sequence according to SEQ ID NO: 243. In some embodiments, one or more helper antigens comprise an Anopheles antigen. In some embodiments, a helper antigen comprises or consists of an Anopheles gambiae TRIO. In some embodiments, an Anopheles gambiae TRIO comprises or consists of an amino acid sequence according to SEQ ID NO: 246.

In some embodiments, a polypeptide encoded by a provided polyribonucleotide comprises a secretory signal and a helper antigen immediately follows the secretory signal.

In some embodiments, a polypeptide encoded by a provided polyribonucleotide comprises a helper antigen at the C-terminus of the polypeptide.

In some embodiments, a polypeptide encoded by a provided polyribonucleotide comprises a multimerization region. In some embodiments, a multimerization region comprises or consists of a trimerization region. In some embodiments, a trimerization region comprises or consists of a fibritin region. In some embodiments, a fibritin region comprises or consists of an amino acid sequence according to SEQ ID NO: 255. In some embodiments, a polypeptide comprises a multimerization region at the N-terminus of the polypeptide.

In some embodiments, a polypeptide encoded by a provided polyribonucleotide comprises a secretory signal. In some embodiments, a secretory signal comprises or consists a Plasmodium secretory signal. In some embodiments, a Plasmodium secretory signal comprises or consists of a Plasmodium CSP secretory signal. In some embodiments, a Plasmodium CSP secretory signal comprises or consists of an amino acid sequence according to SEQ ID NO: 174. In some embodiments, a secretory signal comprises or consists of a heterologous secretory signal. In some embodiments, a heterologous secretory signal comprises or consists of a non-human secretory signal. In some embodiments, a heterologous secretory signal comprises or consists of a viral secretory signal. In some embodiments, a viral secretory signal comprises or consists of an HSV secretory signal. In some embodiments, an HSV secretory signal comprises or consists of an HSV-1 or HSV-2 secretory signal. In some embodiments, an HSV secretory signal comprises or consists of an HSV glycoprotein D (gD) secretory signal. In some embodiments, an HSV gD secretory signal comprises or consists of an amino acid sequence according to SEQ ID NO: 159. In some embodiments, an HSV gD secretory signal comprises or consists of an amino acid sequence according to SEQ ID NO: 165. In some embodiments, a secretory signal comprises or consists of an Ebola virus secretory signal. In some embodiments, an Ebola virus secretory signal comprises or consists of an Ebola virus spike glycoprotein (SGP) secretory signal. In some embodiments, an Ebola virus SGP secretory signal comprises or consists of an amino acid sequence according to SEQ ID NO: 177.

In some embodiments, a secretory signal present in the polypeptide encoded by a provided polyribonucleotide is located at the N-terminus of the polypeptide.

In some embodiments, a polypeptide encoded by a provided polyribonucleotide comprises a transmembrane region. In some embodiments, a transmembrane region comprises or consists of a Plasmodium transmembrane region. In some embodiments, a Plasmodium transmembrane region comprises or consists of a Plasmodium CSP glycosylphosphatidylinositol (GPI) anchor region. In some embodiments, a Plasmodium CSP GPI anchor region comprises or consists of an amino acid sequence according to SEQ ID NO: 231.

In some embodiments, a transmembrane region present in the polypeptide encoded by a provided polyribonucleotide comprises or consists of a heterologous transmembrane region. In some embodiments, a heterologous transmembrane region does not comprise a hemagglutin transmembrane region. In some embodiments, a heterologous transmembrane region comprises or consists of a non-human transmembrane region. In some embodiments, a heterologous transmembrane region comprises or consists of a viral transmembrane region. In some embodiments, a heterologous transmembrane region comprises or consists of an HSV transmembrane region. In some embodiments, an HSV transmembrane region comprises or consists of an HSV-1 or HSV-2 transmembrane region. In some embodiments, an HSV transmembrane region comprises or consists of an HSV gD transmembrane region. In some embodiments, an HSV gD transmembrane region comprises or consists of an amino acid sequence according to SEQ ID NO: 234.

In some embodiments, a transmembrane region present in the polypeptide encoded by a provided polyribonucleotide comprises or consists of a human transmembrane region. In some embodiments, a human transmembrane region comprises or consists of a human decay accelerating factor glycosylphosphatidylinositol (hDAF-GPI) anchor region. In some embodiments, an hDAF-GPI anchor region comprises or consists of an amino acid sequence according to SEQ ID NO: 237.

In some embodiments, a polypeptide encoded by a provided polyribonucleotide does not comprise a secretory signal.

In some embodiments, a polypeptide encoded by a provided polyribonucleotide does not comprise a transmembrane region.

In some embodiments, a polypeptide encoded by a provided polyribonucleotide comprises one or more linkers. In some embodiments, one or more linkers comprise or consist of an amino acid sequence according to SEQ ID NO: 258. In some embodiments, one or more linkers comprise or consist of an amino acid sequence according to SEQ ID NO: 279. In some embodiments, one or more linkers comprise or consist of an amino acid sequence according to SEQ ID NO: 270. In some embodiments, one or more linkers comprise or consist of an amino acid sequence according to SEQ ID NO: 282.

In some embodiments where a transmembrane is present, a polypeptide encoded by a provided polyribonucleotide comprises a linker between the C-terminal region or portion thereof and the transmembrane region.

In some embodiments where a polypeptide encoded by a provided polyribonucleotide comprises an amino acid sequence of NANPNVDP (SEQ ID NO: 102), the polypeptide comprises a linker after an amino acid sequence of NANPNVDP (SEQ ID NO: 102).

In some embodiments, a polypeptide encoded by a provided polyribonucleotide comprises: (i) one or more Plasmodium CSP junction regions, portions thereof, or variants thereof (e.g., according to certain embodiments described herein); (ii) one or more repeats of the amino acid sequence of NANPNVDP (SEQ ID NO: 102) (e.g., according to certain embodiments described herein); (iii) one or more Plasmodium CSP C-terminal regions or portions thereof (e.g., according to certain embodiments described herein), (iv) a secretory signal (e.g., according to certain embodiments described herein), and (v) a transmembrane region (e.g., according to certain embodiments described herein), and wherein the polypeptide does not comprise: (a) an amino acid sequence of NPNA (SEQ ID NO: 141), and (b) a Plasmodium CSP N-terminal region or portion thereof. In some embodiments, a polypeptide does not comprise a Plasmodium CSP N-terminal end region. In some embodiments, a polypeptide comprises one or more Plasmodium CSP N-terminal end regions or portions thereof (e.g., according to certain embodiments described herein). In some embodiments, a polypeptide comprises one or more helper antigens (e.g., according to certain embodiments described herein).

In some embodiments, a polypeptide encoded by a provided polyribonucleotide comprises: (i) a secretory signal (e.g., according to certain embodiments described herein), (ii) a Plasmodium CSP N-terminal end region (e.g., according to certain embodiments described herein), (iii) a Plasmodium CSP junction region (e.g., according to certain embodiments described herein), (iv) nine repeats of the amino acid sequence of NANPNVDP (SEQ ID NO: 102) (e.g., according to certain embodiments described herein), (v) a Plasmodium CSP C-terminal region (e.g., according to certain embodiments described herein), and (vi) five antigenic repeat regions, wherein each antigenic repeat region comprises: (A) a linker (e.g., according to certain embodiments described herein), and (B) a helper antigen (e.g., according to certain embodiments described herein), and wherein the polypeptide does not comprise any of: (a) an amino acid sequence of NPNA (SEQ ID NO: 141), (b) a Plasmodium CSP N-terminal region or portion thereof, and (c) a transmembrane region. In some embodiments, a polypeptide comprises or consists of an amino acid sequence with at least 85%, at least 90%, at least 95%, at least 96%, at least 97%, at least 98%, at least 99%, or 100% sequence identity to an amino acid sequence according to SEQ ID NO: 36.

In some embodiments, a polypeptide encoded by a provided polyribonucleotide comprises: (i) a secretory signal (e.g., according to certain embodiments described herein), (ii) a helper antigen (e.g., according to certain embodiments described herein), (iii) a linker (e.g., according to certain embodiments described herein), (iv) a Plasmodium CSP N-terminal end region (e.g., according to certain embodiments described herein), (v) a Plasmodium CSP junction region (e.g., according to certain embodiments described herein), (vi) nine repeats of the amino acid sequence of NANPNVDP (SEQ ID NO: 102) (e.g., according to certain embodiments described herein), (vii) a Plasmodium CSP C-terminal region (e.g., according to certain embodiments described herein), (viii) a serine-valine sequence immediately following the Plasmodium CSP C-terminal region (e.g., according to certain embodiments described herein), (ix) a linker (e.g., according to certain embodiments described herein), and (x) a transmembrane region (e.g., according to certain embodiments described herein), and wherein the polypeptide does not comprise any of: (a) an amino acid sequence of NPNA (SEQ ID NO: 141), and (b) a Plasmodium CSP N-terminal region or portion thereof. In some embodiments, a polypeptide comprises or consists of an amino acid sequence with at least 85%, at least 90%, at least 95%, at least 96%, at least 97%, at least 98%, at least 99%, or 100% sequence identity to an amino acid sequence according to SEQ ID NO: 39.

In some embodiments, a polypeptide encoded by a provided polyribonucleotide comprises: (i) a secretory signal (e.g., according to certain embodiments described herein), (ii) a portion of a Plasmodium CSP junction region (e.g., according to certain embodiments described herein), (iii) three repeats of the amino acid sequence of NANPNVDP (SEQ ID NO: 102) (e.g., according to certain embodiments described herein), (iv) a Plasmodium CSP C-terminal region (e.g., according to certain embodiments described herein), (v) a serine-valine sequence immediately following the Plasmodium CSP C-terminal region (e.g., according to certain embodiments described herein), (vi) a linker (e.g., according to certain embodiments described herein), and (vii) a transmembrane region (e.g., according to certain embodiments described herein), and wherein the polypeptide does not comprise any of: (a) a Plasmodium CSP N-terminal region or portion thereof, (b) a Plasmodium CSP N-terminal end region or portion thereof, and (c) an amino acid sequence of NPNA (SEQ ID NO: 141). In some embodiments, a polypeptide comprises or consists of an amino acid sequence with at least 85%, at least 90%, at least 95%, at least 96%, at least 97%, at least 98%, at least 99%, or 100% sequence identity to an amino acid sequence according to SEQ ID NO: 57.

In some embodiments, a polypeptide encoded by a provided polyribonucleotide comprises (i) a secretory signal (e.g., according to certain embodiments described herein), (ii) a portion of a Plasmodium CSP junction region (e.g., according to certain embodiments described herein), (iii) nine repeats of the amino acid sequence of NANPNVDP (SEQ ID NO: 102) (e.g., according to certain embodiments described herein), (iv) a Plasmodium CSP C-terminal region (e.g., according to certain embodiments described herein), (v) a serine-valine sequence immediately following the Plasmodium CSP C-terminal region (e.g., according to certain embodiments described herein), (vi) a linker (e.g., according to certain embodiments described herein), and (vii) a transmembrane region (e.g., according to certain embodiments described herein), and wherein the polypeptide does not comprise any of: (a) a Plasmodium CSP N-terminal region or portion thereof, (b) a Plasmodium CSP N-terminal end region or portion thereof, and (c) an amino acid sequence of NPNA (SEQ ID NO: 141). In some embodiments, a polypeptide comprises or consists of an amino acid sequence with at least 85%, at least 90%, at least 95%, at least 96%, at least 97%, at least 98%, at least 99%, or 100% sequence identity to an amino acid sequence according to SEQ ID NO: 60.

In some embodiments, a polypeptide encoded by a provided polyribonucleotide comprises (i) a secretory signal (e.g., according to certain embodiments described herein), (ii) a Plasmodium CSP junction region (e.g., according to certain embodiments described herein), (iii) three repeats of the amino acid sequence of NANPNVDP (SEQ ID NO: 102) (e.g., according to certain embodiments described herein), (iv) a Plasmodium CSP C-terminal region (e.g., according to certain embodiments described herein), (v) a serine-valine sequence immediately following the Plasmodium CSP C-terminal region (e.g., according to certain embodiments described herein), (vi) a linker (e.g., according to certain embodiments described herein), and (vii) a transmembrane region (e.g., according to certain embodiments described herein), and wherein the polypeptide does not comprise any of: (a) a Plasmodium CSP N-terminal region or portion thereof, (b) a Plasmodium CSP N-terminal end region or portion thereof, and (c) an amino acid sequence of NPNA (SEQ ID NO: 141). In some embodiments, a polypeptide comprises or consists of an amino acid sequence with at least 85%, at least 90%, at least 95%, at least 96%, at least 97%, at least 98%, at least 99%, or 100% sequence identity to an amino acid sequence according to SEQ ID NO: 63.

In some embodiments, a polypeptide encoded by a provided polyribonucleotide comprises (i) a secretory signal (e.g., according to certain embodiments described herein), (ii) a Plasmodium CSP junction region (e.g., according to certain embodiments described herein), (iii) nine repeats of the amino acid sequence of NANPNVDP (SEQ ID NO: 102) (e.g., according to certain embodiments described herein), (iv) a Plasmodium CSP C-terminal region (e.g., according to certain embodiments described herein), (v) a serine-valine sequence immediately following the Plasmodium CSP C-terminal region (e.g., according to certain embodiments described herein), (vi) a linker (e.g., according to certain embodiments described herein), and (vii) a transmembrane region (e.g., according to certain embodiments described herein), and wherein the polypeptide does not comprise any of: (a) a Plasmodium CSP N-terminal region or portion thereof, (b) a Plasmodium CSP N-terminal end region or portion thereof, and (c) an amino acid sequence of NPNA (SEQ ID NO: 141). In some embodiments, a polypeptide comprises or consists of an amino acid sequence with at least 85%, at least 90%, at least 95%, at least 96%, at least 97%, at least 98%, at least 99%, or 100% sequence identity to an amino acid sequence according to SEQ ID NO: 66.

In some embodiments, a polypeptide encoded by a provided polyribonucleotide comprises (i) a secretory signal (e.g., according to certain embodiments described herein), (ii) a Plasmodium CSP junction region variant (e.g., according to certain embodiments described herein), (iii) three repeats of the amino acid sequence of NANPNVDP (SEQ ID NO: 102) (e.g., according to certain embodiments described herein), (iv) a Plasmodium CSP C-terminal region (e.g., according to certain embodiments described herein), (v) a serine-valine sequence immediately following the Plasmodium CSP C-terminal region (e.g., according to certain embodiments described herein), (vi) a linker (e.g., according to certain embodiments described herein), and (vii) a transmembrane region (e.g., according to certain embodiments described herein), and wherein the polypeptide does not comprise any of: (a) a Plasmodium CSP N-terminal region or portion thereof, (b) a Plasmodium CSP N-terminal end region or portion thereof, and (c) an amino acid sequence of NPNA (SEQ ID NO: 141). In some embodiments, a polypeptide comprises or consists of an amino acid sequence with at least 85%, at least 90%, at least 95%, at least 96%, at least 97%, at least 98%, at least 99%, or 100% sequence identity to an amino acid sequence according to SEQ ID NO: 69.

In some embodiments, a polypeptide encoded by a provided polyribonucleotide comprises (i) a secretory signal (e.g., according to certain embodiments described herein), (ii) a Plasmodium CSP junction region variant (e.g., according to certain embodiments described herein), (iii) nine repeats of the amino acid sequence of NANPNVDP (SEQ ID NO: 102) (e.g., according to certain embodiments described herein), (iv) a Plasmodium CSP C-terminal region (e.g., according to certain embodiments described herein), (v) a serine-valine sequence immediately following the Plasmodium CSP C-terminal region (e.g., according to certain embodiments described herein), (vi) a linker (e.g., according to certain embodiments described herein), and (vii) a transmembrane region (e.g., according to certain embodiments described herein), and wherein the polypeptide does not comprise any of: (a) a Plasmodium CSP N-terminal region or portion thereof, (b) a Plasmodium CSP N-terminal end region or portion thereof, and (c) an amino acid sequence of NPNA (SEQ ID NO: 141). In some embodiments, a polypeptide comprises or consists of an amino acid sequence with at least 85%, at least 90%, at least 95%, at least 96%, at least 97%, at least 98%, at least 99%, or 100% sequence identity to an amino acid sequence according to SEQ ID NO: 72.

In some embodiments, a polypeptide encoded by a provided polyribonucleotide comprises: (i) a secretory signal (e.g., according to certain embodiments described herein), (ii) a portion of a Plasmodium CSP junction region (e.g., according to certain embodiments described herein), (iii) three repeats of the amino acid sequence of NANPNVDP (SEQ ID NO: 102) (e.g., according to certain embodiments described herein), (iv) a Plasmodium CSP C-terminal region (e.g., according to certain embodiments described herein), (v) a serine-valine sequence immediately following the Plasmodium CSP C-terminal region (e.g., according to certain embodiments described herein), (vi) a linker (e.g., according to certain embodiments described herein), and (vii) a transmembrane region (e.g., according to certain embodiments described herein), and wherein the polypeptide does not comprise any of: (a) a Plasmodium CSP N-terminal region or portion thereof, (b) a Plasmodium CSP N-terminal end region or portion thereof, and (c) an amino acid sequence of NPNA (SEQ ID NO: 141). In some embodiments, a polypeptide comprises or consists of an amino acid sequence with at least 85%, at least 90%, at least 95%, at least 96%, at least 97%, at least 98%, at least 99%, or 100% sequence identity to an amino acid sequence according to SEQ ID NO: 75.

In some embodiments, a polypeptide encoded by a provided polyribonucleotide comprises: (i) a secretory signal (e.g., according to certain embodiments described herein), (ii) a portion of a Plasmodium CSP junction region (e.g., according to certain embodiments described herein), (iii) nine repeats of the amino acid sequence of NANPNVDP (SEQ ID NO: 102) (e.g., according to certain embodiments described herein), (iv) a Plasmodium CSP C-terminal region (e.g., according to certain embodiments described herein), (v) a serine-valine sequence immediately following the Plasmodium CSP C-terminal region (e.g., according to certain embodiments described herein), (vi) a linker (e.g., according to certain embodiments described herein), and (vii) a transmembrane region (e.g., according to certain embodiments described herein), and wherein the polypeptide does not comprise any of: (a) a Plasmodium CSP N-terminal region or portion thereof, (b) a Plasmodium CSP N-terminal end region or portion thereof, and (c) an amino acid sequence of NPNA (SEQ ID NO: 141). In some embodiments, a polypeptide comprises or consists of an amino acid sequence with at least 85%, at least 90%, at least 95%, at least 96%, at least 97%, at least 98%, at least 99%, or 100% sequence identity to an amino acid sequence according to SEQ ID NO: 78.

In some embodiments, a polypeptide encoded by a provided polyribonucleotide comprises: (i) a secretory signal (e.g., according to certain embodiments described herein), (ii) a Plasmodium CSP N-terminal end region (e.g., according to certain embodiments described herein), (iii) a Plasmodium CSP junction region (e.g., according to certain embodiments described herein), (iv) nine repeats of the amino acid sequence of NANPNVDP (SEQ ID NO: 102) (e.g., according to certain embodiments described herein), (v) a Plasmodium CSP C-terminal region (e.g., according to certain embodiments described herein), (vi) a serine-valine sequence immediately following the Plasmodium CSP C-terminal region (e.g., according to certain embodiments described herein), (vii) a linker (e.g., according to certain embodiments described herein), and (viii) a transmembrane region (e.g., according to certain embodiments described herein), and wherein the polypeptide does not comprise any of: (a) a Plasmodium CSP N-terminal region or portion thereof, and (b) an amino acid sequence of NPNA (SEQ ID NO: 141). In some embodiments, a polypeptide comprises or consists of an amino acid sequence with at least 85%, at least 90%, at least 95%, at least 96%, at least 97%, at least 98%, at least 99%, or 100% sequence identity to an amino acid sequence according to SEQ ID NO: 81.

In some embodiments, a polypeptide encoded by a provided polyribonucleotide comprises: (i) a secretory signal (e.g., according to certain embodiments described herein), (ii) a Plasmodium CSP N-terminal end region (e.g., according to certain embodiments described herein), (iii) a Plasmodium CSP junction region variant (e.g., according to certain embodiments described herein), (iv) nine repeats of the amino acid sequence of NANPNVDP (SEQ ID NO: 102) (e.g., according to certain embodiments described herein), (v) a Plasmodium CSP C-terminal region (e.g., according to certain embodiments described herein), (vi) a serine-valine sequence immediately following the Plasmodium CSP C-terminal region (e.g., according to certain embodiments described herein), (vii) a linker (e.g., according to certain embodiments described herein), and (viii) a transmembrane region (e.g., according to certain embodiments described herein), and wherein the polypeptide does not comprise any of: (a) a Plasmodium CSP N-terminal region or portion thereof, and (b) an amino acid sequence of NPNA (SEQ ID NO: 141). In some embodiments, a polypeptide comprises or consists of an amino acid sequence with at least 85%, at least 90%, at least 95%, at least 96%, at least 97%, at least 98%, at least 99%, or 100% sequence identity to an amino acid sequence according to SEQ ID NO: 84.

In some embodiments, a polypeptide encoded by a provided polyribonucleotide comprises (i) a secretory signal (e.g., according to certain embodiments described herein), (ii) a Plasmodium CSP N-terminal end region (e.g., according to certain embodiments described herein), (iii) a Plasmodium CSP junction region (e.g., according to certain embodiments described herein), (iv) nine repeats of the amino acid sequence of NANPNVDP (SEQ ID NO: 102) (e.g., according to certain embodiments described herein), (v) a Plasmodium CSP C-terminal region (e.g., according to certain embodiments described herein), (vi) a transmembrane region (e.g., according to certain embodiments described herein), and wherein the polypeptide does not comprise any of: (a) a Plasmodium CSP N-terminal region or portion thereof, and (b) an amino acid sequence of NPNA (SEQ ID NO: 141). In some embodiments, a polypeptide comprises or consists of an amino acid sequence with at least 85%, at least 90%, at least 95%, at least 96%, at least 97%, at least 98%, at least 99%, or 100% sequence identity to an amino acid sequence according to SEQ ID NO: 96.

In some embodiments, a polypeptide encoded by a provided polyribonucleotide comprises (i) a secretory signal (e.g., according to certain embodiments described herein), (ii) a Plasmodium CSP N-terminal end region (e.g., according to certain embodiments described herein), (iii) a Plasmodium CSP junction region (e.g., according to certain embodiments described herein), (iv) nine repeats of the amino acid sequence of NANPNVDP (SEQ ID NO: 102) (e.g., according to certain embodiments described herein), (v) a Plasmodium CSP C-terminal region (e.g., according to certain embodiments described herein), and (vi) a transmembrane region (e.g., according to certain embodiments described herein), and wherein the polypeptide does not comprise any of: (a) a Plasmodium CSP N-terminal region or portion thereof, and (b) an amino acid sequence of NPNA (SEQ ID NO: 141). In some embodiments, a polypeptide comprises or consists of an amino acid sequence with at least 85%, at least 90%, at least 95%, at least 96%, at least 97%, at least 98%, at least 99%, or 100% sequence identity to an amino acid sequence according to SEQ ID NO: 99.

In some embodiments, a polypeptide encoded by a provided polyribonucleotide comprises: (i) a secretory signal (e.g., according to certain embodiments described herein), (ii) two or more Plasmodium CSP neutralizing region repeats, wherein each Plasmodium CSP neutralizing region repeat comprises or consists of: (a) a Plasmodium CSP N-terminal end region (e.g., according to certain embodiments described herein), (b) a Plasmodium CSP junction region (e.g., according to certain embodiments described herein), (c) two repeats of the amino acid sequence of NANPNVDP (SEQ ID NO: 102) (e.g., according to certain embodiments described herein), and (d) a linker (e.g., according to certain embodiments described herein), (iii) a portion of a Plasmodium CSP major repeat region (e.g., according to certain embodiments described herein), (iv) a Plasmodium CSP C-terminal region (e.g., according to certain embodiments described herein), (v) a serine-valine sequence immediately following the Plasmodium CSP C-terminal region (e.g., according to certain embodiments described herein), (vi) a linker (e.g., according to certain embodiments described herein), and (vii) a transmembrane region (e.g., according to certain embodiments described herein), and wherein the polypeptide does not comprise a Plasmodium CSP N-terminal region or portion thereof. In some embodiments, a polypeptide comprises exactly four Plasmodium CSP neutralizing region repeats. In some embodiments, a polypeptide comprises or consists of an amino acid sequence with at least 85%, at least 90%, at least 95%, at least 96%, at least 97%, at least 98%, at least 99%, or 100% sequence identity to an amino acid sequence according to SEQ ID NO: 87.

In some embodiments, a polypeptide encoded by a provided polyribonucleotide comprises: (i) a secretory signal (e.g., according to certain embodiments described herein), (ii) one Plasmodium CSP junction region (e.g., according to certain embodiments described herein), (iii) three repeats of the amino acid sequence of NANPNVDP (SEQ ID NO: 102) (e.g., according to certain embodiments described herein), (iv) a Plasmodium CSP major repeat region (e.g., according to certain embodiments described herein), (v) one Plasmodium CSP C-terminal region (e.g., according to certain embodiments described herein), (vi) a serine-valine sequence immediately following the Plasmodium CSP C-terminal region (e.g., according to certain embodiments described herein), (vii) a linker (e.g., according to certain embodiments described herein), and (viii) a transmembrane region (e.g., according to certain embodiments described herein), and wherein the polypeptide does not comprise any of: (a) a Plasmodium CSP N-terminal region or portion thereof, and (b) a Plasmodium CSP N-terminal end region or portion thereof. In some embodiments, a polypeptide comprises or consists of an amino acid sequence with at least 85%, at least 90%, at least 95%, at least 96%, at least 97%, at least 98%, at least 99%, or 100% sequence identity to an amino acid sequence according to SEQ ID NO: 30.

In some embodiments, a polypeptide encoded by a provided polyribonucleotide comprises: (i) a secretory signal (e.g., according to certain embodiments described herein), (ii) a Plasmodium CSP N-terminal region (e.g., according to certain embodiments described herein), (iii) a Plasmodium CSP N-terminal end region (e.g., according to certain embodiments described herein), (iv) a portion of a Plasmodium CSP junction region (e.g., according to certain embodiments described herein), (v) three repeats of the amino acid sequence of NANPNVDP (SEQ ID NO: 102) (e.g., according to certain embodiments described herein), (vi) a Plasmodium CSP major repeat region (e.g., according to certain embodiments described herein), (vii) a Plasmodium CSP C-terminal region (e.g., according to certain embodiments described herein), and (viii) a serine immediately following the Plasmodium CSP C-terminal region (e.g., according to certain embodiments described herein), and wherein the polypeptide does not comprise a transmembrane region. In some embodiments, a polypeptide comprises or consists of an amino acid sequence with at least 85%, at least 90%, at least 95%, at least 96%, at least 97%, at least 98%, at least 99%, or 100% sequence identity to an amino acid sequence according to SEQ ID NO: 27.

In some embodiments, a polypeptide encoded by a provided polyribonucleotide comprises: (i) a secretory signal (e.g., according to certain embodiments described herein), (ii) a Plasmodium CSP N-terminal region (e.g., according to certain embodiments described herein), (iii) a Plasmodium CSP N-terminal end region (e.g., according to certain embodiments described herein), (iv) a Plasmodium CSP junction region (e.g., according to certain embodiments described herein), (v) three repeats of the amino acid sequence of NANPNVDP (SEQ ID NO: 102) (e.g., according to certain embodiments described herein), (vi) a Plasmodium CSP major repeat region (e.g., according to certain embodiments described herein), (vii) a Plasmodium CSP C-terminal region (e.g., according to certain embodiments described herein), and (viii) a serine immediately following the Plasmodium CSP C-terminal region (e.g., according to certain embodiments described herein), and wherein the polypeptide does not comprise a transmembrane region. In some embodiments, a polypeptide comprises or consists of an amino acid sequence at least 85%, at least 90%, at least 95%, at least 96%, at least 97%, at least 98%, at least 99%, or 100% sequence identity to an amino acid sequence according to SEQ ID NO: 6.

In some embodiments, a polypeptide encoded by a provided polyribonucleotide comprises: (i) a secretory signal (e.g., according to certain embodiments described herein), (ii) a Plasmodium CSP N-terminal region (e.g., according to certain embodiments described herein), (iii) a Plasmodium CSP N-terminal end region (e.g., according to certain embodiments described herein), (iv) a Plasmodium CSP junction region (e.g., according to certain embodiments described herein), (v) three repeats of the amino acid sequence of NANPNVDP (SEQ ID NO: 102) (e.g., according to certain embodiments described herein), (vi) a Plasmodium CSP major repeat region (e.g., according to certain embodiments described herein), (vii) a Plasmodium CSP C-terminal region (e.g., according to certain embodiments described herein), and (viii) a serine immediately following the Plasmodium CSP C-terminal region (e.g., according to certain embodiments described herein), and wherein the polypeptide does not comprise a transmembrane region. In some embodiments, a polypeptide comprises or consists of an amino acid sequence with at least 85%, at least 90%, at least 95%, at least 96%, at least 97%, at least 98%, at least 99%, or 100% sequence identity to an amino acid sequence according to SEQ ID NO: 24.

In some embodiments, a polypeptide encoded by a provided polyribonucleotide comprises: (i) a secretory signal (e.g., according to certain embodiments described herein), (ii) a Plasmodium CSP N-terminal region (e.g., according to certain embodiments described herein), (iii) a Plasmodium CSP N-terminal end region (e.g., according to certain embodiments described herein), (iv) a Plasmodium CSP junction region (e.g., according to certain embodiments described herein), (v) three repeats of the amino acid sequence of NANPNVDP (SEQ ID NO: 102) (e.g., according to certain embodiments described herein), (vi) a Plasmodium CSP major repeat region (e.g., according to certain embodiments described herein), (vii) a Plasmodium CSP C-terminal region (e.g., according to certain embodiments described herein), and (viii) a serine-valine sequence immediately following the Plasmodium CSP C-terminal region (e.g., according to certain embodiments described herein), and wherein the polypeptide does not comprise a transmembrane region. In some embodiments, a polypeptide comprises or consists of an amino acid sequence with at least 85%, at least 90%, at least 95%, at least 96%, at least 97%, at least 98%, at least 99%, or 100% sequence identity to an amino acid sequence according to SEQ ID NO: 93.

In some embodiments, a polypeptide encoded by a provided polyribonucleotide comprises: (i) a secretory signal (e.g., according to certain embodiments described herein), (ii) a Plasmodium CSP N-terminal region (e.g., according to certain embodiments described herein), (iii) a Plasmodium CSP N-terminal end region (e.g., according to certain embodiments described herein), (iv) a Plasmodium CSP junction region (e.g., according to certain embodiments described herein), (v) three repeats of the amino acid sequence of NANPNVDP (SEQ ID NO: 102) (e.g., according to certain embodiments described herein), (vi) a Plasmodium CSP major repeat region (e.g., according to certain embodiments described herein), (vii) a Plasmodium CSP C-terminal region (e.g., according to certain embodiments described herein), and (viii) a transmembrane region (e.g., according to certain embodiments described herein). In some embodiments, a polypeptide comprises or consists of an amino acid sequence with at least 85%, at least 90%, at least 95%, at least 96%, at least 97%, at least 98%, at least 99%, or 100% sequence identity to an amino acid sequence according to SEQ ID NO: 33.

In some embodiments, a polypeptide encoded by a provided polyribonucleotide comprises: (i) a secretory signal (e.g., according to certain embodiments described herein), (ii) a Plasmodium CSP N-terminal region (e.g., according to certain embodiments described herein), (iii) a Plasmodium CSP N-terminal end region (e.g., according to certain embodiments described herein), (iv) a Plasmodium CSP junction region (e.g., according to certain embodiments described herein), (v) three repeats of the amino acid sequence of NANPNVDP (SEQ ID NO: 102) (e.g., according to certain embodiments described herein), (vi) a Plasmodium CSP major repeat region (e.g., according to certain embodiments described herein), (vii) a Plasmodium CSP C-terminal region (e.g., according to certain embodiments described herein), (viii) a serine-valine sequence immediately following the Plasmodium CSP C-terminal region (e.g., according to certain embodiments described herein), (ix) a linker (e.g., according to certain embodiments described herein), (x) a multimerization region (e.g., according to certain embodiments described herein), and wherein the polypeptide does not comprise a transmembrane region. In some embodiments, a polypeptide comprises or consists of an amino acid sequence with at least 85%, at least 90%, at least 95%, at least 96%, at least 97%, at least 98%, at least 99%, or 100% sequence identity to an amino acid sequence according to SEQ ID NO: 42.

In some embodiments, a polypeptide encoded by a provided polyribonucleotide comprises: (i) a secretory signal (e.g., according to certain embodiments described herein), (ii) a Plasmodium CSP N-terminal region (e.g., according to certain embodiments described herein), (iii) a Plasmodium CSP N-terminal end region (e.g., according to certain embodiments described herein), (iv) a Plasmodium CSP junction region (e.g., according to certain embodiments described herein), (v) three repeats of the amino acid sequence of NANPNVDP (SEQ ID NO: 102) (e.g., according to certain embodiments described herein), (vi) a Plasmodium CSP major repeat region (e.g., according to certain embodiments described herein), (vii) a Plasmodium CSP C-terminal region (e.g., according to certain embodiments described herein), (viii) a serine immediately following the Plasmodium CSP C-terminal region (e.g., according to certain embodiments described herein), (ix) a linker (e.g., according to certain embodiments described herein), and (x) a transmembrane region (e.g., according to certain embodiments described herein). In some embodiments, a polypeptide comprises or consists of an amino acid sequence with at least 85%, at least 90%, at least 95%, at least 96%, at least 97%, at least 98%, at least 99%, or 100% sequence identity to an amino acid sequence according to SEQ ID NO: 48.

In some embodiments, a polypeptide encoded by a provided polyribonucleotide comprises: (i) a secretory signal (e.g., according to certain embodiments described herein), (ii) a Plasmodium CSP N-terminal region (e.g., according to certain embodiments described herein), (iii) a Plasmodium CSP N-terminal end region (e.g., according to certain embodiments described herein), (iv) a Plasmodium CSP junction region (e.g., according to certain embodiments described herein), (v) three repeats of the amino acid sequence of NANPNVDP (SEQ ID NO: 102) (e.g., according to certain embodiments described herein), (vi) a Plasmodium CSP major repeat region (e.g., according to certain embodiments described herein), (vii) a Plasmodium CSP C-terminal region (e.g., according to certain embodiments described herein), (viii) a serine-valine sequence immediately following the Plasmodium CSP C-terminal region (e.g., according to certain embodiments described herein), (ix) a linker (e.g., according to certain embodiments described herein), (x) a transmembrane region (e.g., according to certain embodiments described herein). In some embodiments, a polypeptide comprises or consists of an amino acid sequence with at least 85%, at least 90%, at least 95%, at least 96%, at least 97%, at least 98%, at least 99%, or 100% sequence identity to an amino acid sequence according to SEQ ID NO: 90.

In some embodiments, a polypeptide encoded by a provided polyribonucleotide comprises: (i) a secretory signal (e.g., according to certain embodiments described herein), (ii) a Plasmodium CSP N-terminal region (e.g., according to certain embodiments described herein), (iii) a Plasmodium CSP N-terminal end region (e.g., according to certain embodiments described herein), (iv) a Plasmodium CSP junction region (e.g., according to certain embodiments described herein), (v) three repeats of the amino acid sequence of NANPNVDP (SEQ ID NO: 102) (e.g., according to certain embodiments described herein), (vi) a Plasmodium CSP major repeat region (e.g., according to certain embodiments described herein), (vii) a Plasmodium CSP C-terminal region (e.g., according to certain embodiments described herein), (viii) a serine immediately following the Plasmodium CSP C-terminal region (e.g., according to certain embodiments described herein), and (ix) a transmembrane region (e.g., according to certain embodiments described herein). In some embodiments, a polypeptide comprises or consists of an amino acid sequence with at least 85%, at least 90%, at least 95%, at least 96%, at least 97%, at least 98%, at least 99%, or 100% sequence identity to an amino acid sequence according to SEQ ID NO: 21.

In some embodiments, when present, features (i) to (x) (as referred to above) are in a polypeptide in numerical order from the C-terminus to the N-terminus.

In some embodiments, Plasmodium is Plasmodium falciparum. In some embodiments, one or more Plasmodium CSP polypeptide regions or portions thereof present in the polypeptide of a provide polyribonucleotide are one or more P. falciparum CSP polypeptide regions or portions thereof. In some embodiments, Plasmodium falciparum is Plasmodium falciparum isolate 3D7.

In some embodiments, a provided polyribonucleotide is an isolated polyribonucleotide. In some embodiments, a provided polyribonucleotide is an engineered polyribonucleotide. In some embodiments, a provided polyribonucleotide is a codon-optimized polyribonucleotide.

In one aspect, provided herein is an RNA construct comprising a polyribonucleotide described herein. In some embodiments, an RNA construct comprises in 5′ to 3′ order: (i) a 5′ UTR that comprises or consists of a modified human alpha-globin 5′-UTR; (ii) a polyribonucleotide as described herein; (iii) a 3′ UTR that comprises or consists of a first sequence from the amino terminal enhancer of split (AES) messenger RNA and a second sequence from the mitochondrial encoded 12S ribosomal RNA; and (iv) a polyA tail sequence. In some embodiments, a 5′ UTR comprises or consists of a ribonucleic acid sequence according to SEQ ID NO: 415. In some embodiments, a 3′ UTR comprises or consists of a ribonucleic acid sequence according to SEQ ID NO: 416. In some embodiments, a polyA tail sequence is a split polyA tail sequence. In some embodiments, a split polyA tail sequence comprises or consists of a ribonucleic acid sequence according to SEQ ID NO: 417.

In some embodiments, a provided RNA construct further comprises a 5′ cap. In some embodiments, a provided RNA construct further comprises a cap proximal sequence comprising positions +1, +2, +3, +4, and +5 of the polyribonucleotide. In some embodiments, a 5′ cap comprises or consists of a Cap1 structure comprising m7 (3′-OMeG) (5′) ppp (5′) (2′-OMeA1) pG2, wherein A₁ is position +1 of the polyribonucleotide, and G₂ is position +2 of the polyribonucleotide. In some embodiments, a cap proximal sequence comprises A₁ and G₂ of the Cap1 structure, and a sequence comprising: A₃A₄U₅ (SEQ ID NO: 424) at positions +3, +4 and +5 respectively of the polyribonucleotide.

In some embodiments, a provided polyribonucleotide includes modified uridines in place of all uridines. In some embodiments, modified uridines are each N1-methyl-pseudouridine.

Compositions comprising provided polynucleotides or provided RNA constructs are also within the scope of the present disclosure. In some embodiments, such a composition further comprises lipid nanoparticles, polyplexes (PLX), lipidated polyplexes (LPLX), or liposomes. In some embodiments, one or more polyribonucleotides or one or more RNA constructs are fully or partially encapsulated within the lipid nanoparticles, polyplexes (PLX), lipidated polyplexes (LPLX), or liposomes. In some embodiments, a composition further comprises lipid nanoparticles, wherein the one or more polyribonucleotides or the one or more RNA constructs are fully or partially encapsulated within the lipid nanoparticles. In some embodiments, lipid nanoparticles target liver cells. In some embodiments, lipid nanoparticles target secondary lymphoid organ cells. In some embodiments, lipid nanoparticles are cationic lipid nanoparticles. In some embodiments, lipid nanoparticles each comprise: (a) a polymer-conjugated lipid; (b) a cationically ionizable lipid; and (c) one or more neutral lipids. In some embodiments, a polymer-conjugated lipid comprises a PEG-conjugated lipid. In some embodiments, a polymer-conjugated lipid comprises 2-[(polyethylene glycol)-2000]-N,N-ditetradecylacetamide. In some embodiments, one or more neutral lipids comprise 1,2-Distearoyl-sn-glycero-3-phosphocholine (DPSC). In some embodiments, one or more neutral lipids comprise cholesterol. In some embodiments, a cationically ionizable lipid comprises [(4-Hydroxybutyl) azanediyl]di(hexane-6,1-diyl)bis(2-hexyldecanoate). In some embodiments, lipid nanoparticles have an average diameter of about 50-150 nm.

Another aspect provided herein relates to a pharmaceutical composition comprising a composition as described herein and at least one pharmaceutically acceptable excipient. In some embodiments, a pharmaceutical composition comprises a cryoprotectant, optionally wherein a cryoprotectant is sucrose. In some embodiments, a pharmaceutical composition comprises an aqueous buffered solution, optionally wherein an aqueous buffered solution comprises one or more of Tris base, Tris HCl, NaCl, KCl, Na₂HPO₄, and KH₂PO₄.

A further aspect provided herein relates to a combination comprising: (i) a first pharmaceutical composition comprising a first polyribonucleotide, wherein the first polyribonucleotide encodes a first polypeptide, and the first polypeptide comprises one or more Plasmodium CSP polypeptide regions or portions thereof; and (ii) a second pharmaceutical composition comprising a second polyribonucleotide, wherein the second polyribonucleotide encodes a second polypeptide, and the second polypeptide comprises one or more Plasmodium T-cell antigens. In some embodiments, a first polyribonucleotide is a polyribonucleotide according to certain embodiments described herein or an RNA construct according to certain embodiments described herein.

Methods of administering to a subject a provided polyribonucleotide, a provided RNA construct, a provided composition, or a provided pharmaceutical composition are also within the scope of the present disclosure. In some embodiments, a method comprises administering to a subject one or more doses of a pharmaceutical composition described herein.

In one aspect, a pharmaceutical composition as described herein for use in the treatment of a malaria infection comprising administering one or more doses of the pharmaceutical composition to a subject. In another aspect, a pharmaceutical composition as described herein for use in the prevention of a malaria infection comprising administering one or more doses of the pharmaceutical composition to a subject.

In some embodiments, two or more doses of the pharmaceutical composition as described herein are administered to a subject. In some embodiments, three or more doses of the pharmaceutical composition as described herein are administered to a subject. In some embodiments, a second of three or more doses is administered to a subject at least 4 weeks after a first of the three or more doses is administered to the subject. In some embodiments, a third of three or more doses is administered to a subject at least 4 weeks after the second of the three or more doses is administered to the subject.

In some embodiments, a fourth dose of the pharmaceutical composition as described herein is administered to a subject. In some embodiments, a fourth dose is administered to a subject at least one year after a third of three or more doses is administered to the subject.

A method comprising administering to a subject a combination according to certain embodiments described herein is also provided herein. In some embodiments, a first pharmaceutical composition and a second pharmaceutical composition are administered on the same day. In some embodiments, a first pharmaceutical composition and a second pharmaceutical composition are administered on different days. In some embodiments, a first pharmaceutical composition and a second pharmaceutical composition are administered to a subject at different locations on a subject's body.

In some embodiments, technologies described herein can be useful for treating a malaria infection. In some embodiments, technologies described herein can be useful for preventing a malaria infection. In some embodiments, a subject that is amenable to technologies described herein has or is at risk of developing a malaria infection. In some embodiments, a subject that is amenable to technologies described herein is a human.

In some embodiments, administration of a composition described herein induces an anti-malaria immune response in the subject. In some embodiments, an anti-malaria immune response in the subject comprises an adaptive immune response. In some embodiments, an anti-malaria immune response in the subject comprises a T-cell response. In some embodiments, a T-cell response is or comprises a CD4+ T cell response. In some embodiments, a T-cell response is or comprises a CD8+ T cell response. In some embodiments, an anti-malaria immune response comprises a B-cell response. In some embodiments, an anti-malaria immune response comprises production of antibodies directed against one or more Plasmodium antigens.

Also within the scope of the present disclosure are a use of a pharmaceutical composition as described herein in the treatment of a malaria infection, a use of a pharmaceutical composition as described herein in the prevention of a malaria infection, and a use of a pharmaceutical composition as described herein in inducing an anti-malaria immune response in a subject.

Also within the scope of the present disclosure are polypeptides encoded by polyribonucleotides according to various embodiments described herein, polypeptides encoded by RNA construct according to various embodiments described herein, host cells comprising polyribonucleotides described herein, host cells comprising RNA constructs described herein, and host cells comprising polypeptides described herein.

### BRIEF DESCRIPTION OF THE DRAWING

FIGS. 1A-1B depict in vitro expression of non-formulated RNA constructs encoding different Plasmodium polypeptide constructs in HEK293T cells. FIG. 1A shows transfection rate as measured by percentage of total HEK293T population that is positive for presence of expressed protein. FIG. 1B shows total expression as measured by median fluorescence of the total HEK293T population for both transfected and non-transfected cells. Permeabilized cells show total protein expressed (black bar, intracellular staining) and non-permeabilized cells show only surface expressed protein (grey bar, surface staining). Each sample was stained in triplicate, bar is a representation of mean with SD; NT, non-transfected.

FIGS. 2A-2C depict in vitro expression of formulated RNA constructs in HEK293T cells. FIG. 2A shows transfection rate as measured by percentage of total HEK293T population that is positive for presence of expressed protein. FIG. 2B shows total expression as measured by median fluorescence of the total HEK293T population for both transfected and non-transfected cells. Permeabilized cells show total protein expressed (black bar, intracellular staining) and non-permeabilized cells show only surface expressed protein (grey bar, surface staining). Each sample was stained in triplicate, bar is a representation of mean with SD. FIG. 2C shows amount of protein detected in culture supernatant where each data point represents a triplicate repeat; NT, non-transfected.

FIGS. 3A-3B depict in vitro expression of non-formulated RNA constructs 59 and 60 encoding different Plasmodium polypeptides in HEK293T cells. FIG. 3A shows transfection rate as measured by percentage of total HEK293T population that is positive for presence of expressed protein. FIG. 3B shows total expression as measured by median fluorescence of the total HEK293T population for both transfected and non-transfected cells. Permeabilized cells show total protein expressed (black bar, intracellular staining) and non-permeabilized cells show only surface expressed protein (grey bar, surface staining). Protein was detected using anti-PfCSP 2A10 antibody. Each sample was stained in triplicate, bar is a representation of mean with SD; NT, non-transfected.

FIGS. 4A-4B depict in vitro expression of non-formulated RNA constructs 91, 100 and 104 encoding different Plasmodium polypeptides in HEK293T cells. FIG. 4A shows transfection rate as measured by percentage of total HEK293T population that is positive for presence of expressed protein. FIG. 4B shows total expression as measured by median fluorescence of the total HEK293T population for both transfected and non-transfected cells. Permeabilized cells show total protein expressed (black bar, intracellular staining) and non-permeabilized cells show only surface expressed protein (grey bar, surface staining). Protein was detected using anti-PfCSP L9 antibody. Each sample was stained in triplicate, bar is a representation of mean with SD; NT, non-transfected.

FIGS. 5A-5B depict in vitro expression of non-formulated RNA constructs 87 and 88 encoding different Plasmodium polypeptides in HEK293T cells. FIG. 5A shows transfection rate as measured by percentage of total HEK293T population that is positive for presence of expressed protein. FIG. 5B shows total expression as measured by median fluorescence of the total HEK293T population for both transfected and non-transfected cells. Permeabilized cells show total protein expressed (black bar, intracellular staining) and non-permeabilized cells show only surface expressed protein (grey bar, surface staining). Protein was detected using anti-PfCSP L9 antibody. Each sample was stained in triplicate, bar is a representation of mean with SD; NT, non-transfected.

FIGS. 6A-6B depict in vitro expression of formulated RNA constructs 87, 88, 91, 100 and 104 encoding different Plasmodium polypeptides in HEK293T cells. FIG. 6A shows transfection rate as measured by percentage of total HEK293T population that is positive for presence of expressed protein. FIG. 6B shows total expression as measured by median fluorescence of the total HEK293T population for both transfected and non-transfected cells. Permeabilized cells show total protein expressed (black bar, intracellular staining) and non-permeabilized cells show only surface expressed protein (grey bar, surface staining). Protein was detected using anti-PfCSP L9 antibody. Each sample was stained in triplicate, bar is a representation of mean with SD; NT, non-transfected.

FIGS. 7A-7C depict immunogenicity induced in mice by formulated RNA constructs. FIG. 7A shows antibodies to Plasmodium falciparum (Pf) CSP full length protein (“PfCSP-FL”). FIG. 7B shows antibodies to PfCSP C-terminal domain (“PfCSP-C”). Each data point is representative of one mouse and the bar denotes mean with SEM. LDL, lower detection limit. FIG. 7C shows antibodies to the region spanning the end of the N-terminal domain until the end of the minor repeats (“PfCsp-76 to140”).

FIGS. 8A-8B depict immunogenicity induced in mice by formulated RNA constructs 87, 88, 91, 100 and 104. FIG. 8A shows antibodies to Plasmodium falciparum (Pf) CSP full length protein (“PfCSP-FL”). FIG. 8B shows antibodies to PfCSP C-terminal domain (“PfCSP-C term (3D7)”). Each data point is representative of one mouse and the bar denotes mean with SEM. LDL, lower detection limit.

FIGS. 9A-9K depict binding of antibodies generated from mice immunized with different formulated RNA constructs to various epitopes. FIG. 9A shows a visual summary of the data in FIGS. 9B-9K in the form of a heatmap. FIGS. 9B-9K each show bars that are representative of the area under the curve (AUC) created when plotting dilution steps versus ECL signal.

FIG. 10 depicts depicts binding of antibodies generated from mice immunized with different formulated RNA constructs 87, 88, 91, 104, and 100 during challenge studies to various epitopes in a heatmap format . . .

FIGS. 11A-11J depict binding of antibodies generated from mice immunized with different formulated RNA constructs to various epitopes. FIG. 11A-11J each show bars that are representative of the area under the curve (AUC) created when plotting dilution steps versus ECL signal.

FIGS. 12A-12B depict binding specificity of antibodies generated from mice immunized with different formulated RNA constructs to CSP protein in Plasmodium falciparum sporozoite lysates. FIG. 12A shows binding between antibodies (serum dilution 1:1250) and CSP protein in the sporozoite (spz) lysates as assessed by luminescence (cps, counts per second). FIG. 12B shows binding of murine anti-Pfs25 mAb32F81 used as negative control and murine anti-CSP mAb3SP2 used as a positive control.

FIGS. 13A-13C depict assessment of antibodies generated from mice immunized with different formulated RNA constructs for ability to inhibit P. falciparum sporozoite traversal. FIG. 13A shows results as percentage of inhibition of traversal activity (mean with SEM) in comparison to the vehicle control, which was set as 0% inhibition. FIGS. 13B-13C show results from negative control (serum from vehicle mice; FIG. 13B) and positive control (mAb317, an antibody that binds to NANP (SEQ ID NO: 147) repeats of the major repeat region and is known to inhibit traversal; FIG. 13C), with 002, 003, 005, 012, 014, and 018 indicating different experimental runs.

FIGS. 14A-14F depict assessment of antibodies generated from mice immunized with different formulated RNA constructs for ability to inhibit P. falciparum sporozoite infection of primary human hepatocytes. FIGS. 14A-14D show results as percentage of inhibition of infection activity (mean with SEM) in comparison to the vehicle control, which was set as 0% inhibition. FIGS. 14E-14F show results from negative control (serum from vehicle mice; FIG. 14E) and positive control (mAb317, an antibody known to inhibit hepatocyte infection; FIG. 14F).

FIGS. 15A-15E depict assessment of antibodies generated from mice immunized with different formulated RNA constructs for ability to inhibit P. falciparum sporozoite infection of primary human hepatocytes. FIGS. 15A-15C show results as percentage of inhibition of infection activity (mean with SEM) in comparison to the vehicle control, which was set as 0% inhibition. FIGS. 15D-15E show results from negative control (serum from vehicle mice; FIG. 15D) and positive control (mAb317, an antibody known to inhibit hepatocyte infection; FIG. 15E).

FIGS. 16A-16E depict the ability of antibodies generated from mice immunized with different formulated RNA constructs to bind human complement and induce PfCSP sporozoite lysis. Results are showed as living (not-lysed) sporozoites as a percentage of total recorded events. FIGS. 16A-16C represent the same data in increasing dilutions (1:10, 1:100 and 1:1000, respectively). FIG. 16D is representative of the vehicle serum control at the same dilutions. FIG. 16E depicts the result when using a positive control, mAb317 antibody, which binds PfCSP and induces full sporozoite lysis and a negative control, mAb1245 antibody, that binds to a Pf protein that is not expressed in sporozoites and, thus, does not induce sporozoite lysis. Each data point is representative of a technical replicate of pooled serum samples and the bar denotes mean with SEM.

FIGS. 17A-17B depict the binding and dissociation of antibodies generated from mice immunized with different formulated RNA constructs to full length PfCSP and two peptides (junction+minor repeats and major repeats. Serum samples from all animals immunized by the same construct were pooled before the analysis. FIG. 17A shows the level of binding of the antibodies to the respective binding partner in RU. FIG. 17B represents the percentage of residual response, meaning the percentage of antibody: antigen complexes, still measurable after 15 min of dissociation, calculated from the initial binding. RU, relative units.

FIGS. 18A-18E depict activation of T-cells, as assessed by secretion of IFN-γ. IFN-γ secretion was assessed using isolated splenocytes (from mice immunized with different formulated RNA constructs) treated with overlapping peptide pools covering the full length CSP protein (PfCSP_FL_pep; FIG. 18A), peptides of epitopes predicted to present on MHC-I (FIG. 18B), on MHC-II (FIG. 18C), or controls (e.g., negative control: gp70-AH1 (SPSYVYHQF [SEQ ID NO: 425]; (FIG. 18D)), 4 μg/mL; positive control: concanavalin A, 2 μg/mL (FIG. 18E)). Samples were measured in triplicate and negative control was measured in duplicate; each data point represents a single mouse and bars represent the group mean spot-forming units (SFU)±SD per 5×10⁵ splenocyte; ve, vehicle.

FIGS. 19A-19E depict activation of T-cells, as assessed by secretion of TNF-α. TNF-α secretion was assessed using isolated splenocytes (from mice immunized different formulated RNA constructs) treated with overlapping peptide pools covering the full length CSP protein (PfCSP_FL_pep; FIG. 19A), peptides of epitopes predicted to present on MHC-I (FIG. 19B), on MHC-II (FIG. 19C), or controls (e.g., negative control: gp70-AH1 (SPSYVYHQF [SEQ ID NO: 425]; FIG. 19D), 4 μg/mL; positive control: concanavalin A, 2 μg/mL (FIG. 19E)). Samples were measured in triplicate and negative control was measured in duplicate; each data point represents a single mouse and bars represent the group mean spot-forming units (SFU)±SD per 5×10⁵ splenocyte; ve, vehicle.

FIGS. 20A-20E depict activation of T-cells, as assessed by secretion of IL-2. IL-2 secretion was assessed using isolated splenocytes (from mice immunized different formulated RNA constructs) treated with overlapping peptide pools covering the full length CSP protein (PfCSP_FL_pep; FIG. 20A), peptides of epitopes predicted to present on MHC-I (FIG. 20B), on MHC-II (FIG. 20C), or controls (e.g., negative control: gp70-AH1 (SPSYVYHQF [SEQ ID NO: 425]; FIG. 20D), 4 μg/mL; positive control: concanavalin A, 2 μg/mL (FIG. 20E)). Samples were measured in triplicate and negative control was measured in duplicate; each data point represents a single mouse and bars represent the group mean spot-forming units (SFU)±SD per 5×10⁵ splenocyte; ve, vehicle.

FIGS. 21A-21E depict activation of T-cells, as assessed by secretion of IL-2 and IFN-γ. IL-2 and IFN-γ secretion was assessed using isolated splenocytes (from mice immunized different formulated RNA constructs) treated with overlapping peptide pools covering the full length CSP protein (PfCSP_FL_pep; FIG. 21A), peptides of epitopes predicted to present on MHC-I (FIG. 21B), on MHC-II (FIG. 21C), or controls (e.g., negative control: gp70-AH1 (SPSYVYHQF [SEQ ID NO: 425]; FIG. 21D), 4 μg/mL; positive control: concanavalin A, 2 μg/mL (FIG. 21E)). Samples were measured in triplicate and negative control was measured in duplicate; each data point represents a single mouse and bars represent the group mean spot-forming units (SFU)+SD per 5×10⁵ splenocyte; ve, vehicle.

FIGS. 22A-22E depict activation of T-cells, as assessed by secretion of TNF-α and IFN-γ. TNF-α and IFN-γ secretion was assessed using isolated splenocytes (from mice immunized different formulated RNA constructs) treated with overlapping peptide pools covering the full length CSP protein (PfCSP_FL_pep; FIG. 22A), peptides of epitopes predicted to present on MHC-I (FIG. 22B), on MHC-II (FIG. 22C), or controls (e.g., negative control: gp70-AH1 (SPSYVYHQF [SEQ ID NO: 425]; FIG. 22D), 4 μg/mL; positive control: concanavalin A, 2 μg/mL (FIG. 22E)). Samples were measured in triplicate and negative control was measured in duplicate; each data point represents a single mouse and bars represent the group mean spot-forming units (SFU)+SD per 5×10⁵ splenocyte; ve, vehicle.

FIGS. 23A-23E depict activation of T-cells, as assessed by secretion of TNF-α and IL-2. TNF-α and IL-2 secretion was assessed using splenocytes (isolated from mice immunized different formulated RNA constructs) treated with overlapping peptide pools covering the full length CSP protein (PfCSP_FL_pep; FIG. 23A), peptides of epitopes predicted to present on MHC-I (FIG. 23B), on MHC-II (FIG. 23C), or controls (e.g., negative control: gp70-AH1 (SPSYVYHQF [SEQ ID NO: 425]; FIG. 23D), 4 μg/mL; positive control: concanavalin A, 2 μg/mL (FIG. 23E)). Samples were measured in triplicate and negative control was measured in duplicate; each data point represents a single mouse and bars represent the group mean spot-forming units (SFU)+SD per 5×10⁵ splenocyte; ve, vehicle.

FIGS. 24A-24E depict activation of T-cells, as assessed by secretion of TNF-α, IL-2 and IFN-γ. TNF-α, IL-2 and IFN-γ secretion was assessed using isolated splenocytes (from mice immunized different formulated RNA constructs) treated with overlapping peptide pools covering the full length CSP protein (PfCSP_FL_pep; FIG. 24A), peptides of epitopes predicted to present on MHC-I (FIG. 24B), on MHC-II (FIG. 24C), or controls (e.g., negative control: gp70-AH1 (SPSYVYHQF [SEQ ID NO: 425]; FIG. 24D), 4 μg/mL; positive control: concanavalin A, 2 μg/mL (FIG. 24E)). Samples were measured in triplicate and negative control was measured in duplicate; each data point represents a single mouse and bars represent the group mean spot-forming units (SFU)±SD per 5×10⁵ splenocyte; ve, vehicle.

FIGS. 25A-25C depict activation of T-cells, as assessed by secretion of IFN-γ. IFN-γ secretion was assessed using isolated splenocytes (from mice immunized with different formulated RNA constructs) treated with overlapping peptide pools covering the full length CSP protein (FIG. 25A) or controls (e.g., negative control: Trp1, 2 μg/mL (FIG. 25B); positive control: concanavalin A, 2 μg/mL (FIG. 25C)). Samples were measured in triplicate and negative control was measured in duplicate; each data point represents a single mouse and bars represent the group mean spot-forming units (SFU)±SD per 5×10⁵ splenocyte. Each data point in the medium and ConA controls represents the mean of triplicates of a pool of splenocytes from all mice. ve, vehicle.

FIGS. 26A-26C depict activation of T-cells, as assessed by secretion of IL-2. IL-2 secretion was assessed using isolated splenocytes (from mice immunized with different formulated RNA constructs) treated with overlapping peptide pools covering the full length CSP protein (FIG. 26A) or controls (e.g., negative control: Trp1, 2 μg/mL (FIG. 26B); positive control: concanavalin A, 2 μg/mL (FIG. 26C)). Samples were measured in triplicate and negative control was measured in duplicate; each data point represents a single mouse and bars represent the group mean spot-forming units (SFU)±SD per 5×10⁵ splenocyte. Each data point in the medium and ConA controls represents the mean of triplicates of a pool of splenocytes from all mice. ve, vehicle.

FIGS. 27A-27C depict activation of T-cells, as assessed by secretion of TNF-α. TNF-α secretion was assessed using isolated splenocytes (from mice immunized with different formulated RNA constructs) treated with overlapping peptide pools covering the full length CSP protein (FIG. 27A) or controls (e.g., negative control: Trp1, 2 μg/mL (FIG. 27B); positive control: concanavalin A, 2 μg/mL (FIG. 27C)). Samples were measured in triplicate and negative control was measured in duplicate; each data point represents a single mouse and bars represent the group mean spot-forming units (SFU)±SD per 5×10⁵ splenocyte. Each data point in the medium and ConA controls represents the mean of triplicates of a pool of splenocytes from all mice. ve, vehicle.

FIGS. 28A-28C depict activation of T-cells, as assessed by secretion of both IFN-γ and IL-2. IFN-γ+IL-2 secretion was assessed using isolated splenocytes (from mice immunized with different formulated RNA constructs) treated with overlapping peptide pools covering the full length CSP protein (FIG. 28A) or controls (e.g., negative control: Trp1, 2 μg/mL (FIG. 28B); positive control: concanavalin A, 2 μg/mL (FIG. 28C)). Samples were measured in triplicate and negative control was measured in duplicate; each data point represents a single mouse and bars represent the group mean spot-forming units (SFU)±SD per 5×10⁵ splenocyte. Each data point in the medium and ConA controls represents the mean of triplicates of a pool of splenocytes from all mice. ve, vehicle.

FIGS. 29A-29C depict activation of T-cells, as assessed by secretion of both IFN-γ and TNF-α. IFN-γ+ TNF-α secretion was assessed using isolated splenocytes (from mice immunized with different formulated RNA constructs) treated with overlapping peptide pools covering the full length CSP protein (FIG. 29A) or controls (e.g., negative control: Trp1, 2 μg/mL (FIG. 29B); positive control: concanavalin A, 2 μg/mL (FIG. 29C)). Samples were measured in triplicate and negative control was measured in duplicate; each data point represents a single mouse and bars represent the group mean spot-forming units (SFU)±SD per 5×10⁵ splenocyte. Each data point in the medium and ConA controls represents the mean of triplicates of a pool of splenocytes from all mice. ve, vehicle.

FIGS. 30A-30C depict activation of T-cells, as assessed by secretion of both IL-2 and TNF-α. IL-2+ TNF-α secretion was assessed using isolated splenocytes (from mice immunized with different formulated RNA constructs) treated with overlapping peptide pools covering the full length CSP protein (FIG. 30A) or controls (e.g., negative control: Trp1, 2 μg/mL (FIG. 30B); positive control: concanavalin A, 2 μg/mL (FIG. 30C)). Samples were measured in triplicate and negative control was measured in duplicate; each data point represents a single mouse and bars represent the group mean spot-forming units (SFU)±SD per 5×10⁵ splenocyte. Each data point in the medium and ConA controls represents the mean of triplicates of a pool of splenocytes from all mice. ve, vehicle.

FIGS. 31A-31C depict activation of T-cells, as assessed by secretion of IFN-γ, IL-2 and TNF-α. IFN-γ+IL-2+ TNF-α secretion was assessed using isolated splenocytes (from mice immunized with different formulated RNA constructs) treated with overlapping peptide pools covering the full length CSP protein (FIG. 31A) or controls (e.g., negative control: Trp1, 2 μg/mL (FIG. 31B); positive control: concanavalin A, 2 μg/mL (FIG. 31C)). Samples were measured in triplicate and negative control was measured in duplicate; each data point represents a single mouse and bars represent the group mean spot-forming units (SFU)±SD per 5×10⁵ splenocyte. Each data point in the medium and ConA controls represents the mean of triplicates of a pool of splenocytes from all mice. ve, vehicle.

FIGS. 32A-32D depict activation of CD4 T cells only, as assessed by secretion of IFN-γ. IFN-γ secretion was assessed by a fluorospot assay after using MACS separation to isolate CD4⁺T cells (from pools of splenocytes from mice immunized with different formulated RNA constructs). Cells were then incubated with overlapping peptide pools covering the full length CSP protein (FIG. 32A) or controls (e.g., negative control: Trp1, 2 μg/mL (FIG. 32B); positive control: concanavalin A, 2 μg/mL (FIG. 32C); medium control (FIG. 32D)). Pooled samples were measured in triplicate and negative control was measured in duplicate; data points and bars represent the group mean spot-forming units (SFU)±SD per 1×10⁵ CD4 T cells; ve, vehicle.

FIGS. 33A-33D depict activation of CD4 T cells only, as assessed by secretion of IL-2. IL-2 secretion was assessed by a fluorospot assay after using MACS separation to isolate CD4⁺T cells (from pools of splenocytes from mice immunized with different formulated RNA constructs). Cells were then incubated with overlapping peptide pools covering the full length CSP protein (FIG. 33A) or controls (e.g., negative control: Trp1, 2 μg/mL (FIG. 33B); positive control: concanavalin A, 2 μg/mL (FIG. 33C); medium control (FIG. 33D)). Pooled samples were measured in triplicate and negative control was measured in duplicate; data points and bars represent the group mean spot-forming units (SFU)±SD per 1×10⁵ CD4 T cells; ve, vehicle.

FIGS. 34A-34D depict activation of CD4 T cells only, as assessed by secretion of TNF-α. TNF-α secretion was assessed by a fluorospot assay after using MACS separation to isolate CD4⁺T cells (from pools of splenocytes from mice immunized with different formulated RNA constructs). Cells were then incubated with overlapping peptide pools covering the full length CSP protein (FIG. 34A) or controls (e.g., negative control: Trp1, 2 μg/mL (FIG. 34B); positive control: concanavalin A, 2 μg/mL (FIG. 34C); medium control (FIG. 34D)). Pooled samples were measured in triplicate and negative control was measured in duplicate; data points and bars represent the group mean spot-forming units (SFU)±SD per 1×10⁵ CD4 T cells; ve, vehicle.

FIGS. 35A-35D depict activation of CD4 T cells only, as assessed by secretion of both IFN-γ and IL-2. IFN-γ+IL-2 secretion was assessed by a fluorospot assay after using MACS separation to isolate CD4⁺T cells (from pools of splenocytes from mice immunized with different formulated RNA constructs). Cells were then incubated with overlapping peptide pools covering the full length CSP protein (FIG. 35A) or controls (e.g., negative control: Trp1, 2 g/mL (FIG. 35B); positive control: concanavalin A, 2 μg/mL (FIG. 35C); medium control (FIG. 35D)). Pooled samples were measured in triplicate and negative control was measured in duplicate; data points and bars represent the group mean spot-forming units (SFU)±SD per 1×10⁵ CD4 T cells; ve, vehicle.

FIGS. 36A-36D depict activation of CD4 T cells only, as assessed by secretion of both IFN-γ and TNF-α. IFN-γ+ TNF-α secretion was assessed by a fluorospot assay after using MACS separation to isolate CD4⁺T cells (from pools of splenocytes from mice immunized with different formulated RNA constructs). Cells were then incubated with overlapping peptide pools covering the full length CSP protein (FIG. 36A) or controls (e.g., negative control: Trp1, 2 μg/mL (FIG. 36B); positive control: concanavalin A, 2 μg/mL (FIG. 36C); medium control (FIG. 36D)). Pooled samples were measured in triplicate and negative control was measured in duplicate; data points and bars represent the group mean spot-forming units (SFU)±SD per 1×10⁵ CD4 T cells; ve, vehicle.

FIGS. 37A-37D depict activation of CD4 T cells only, as assessed by secretion of both IL-2 and TNF-α. IL-2+ TNF-α secretion was assessed by a fluorospot assay after using MACS separation to isolate CD4⁺T cells (from pools of splenocytes from mice immunized with different formulated RNA constructs). Cells were then incubated with overlapping peptide pools covering the full length CSP protein (FIG. 37A) or controls (e.g., negative control: Trp1, 2 μg/mL (FIG. 37B); positive control: concanavalin A, 2 μg/mL (FIG. 37C); medium control (FIG. 37D)). Pooled samples were measured in triplicate and negative control was measured in duplicate; data points and bars represent the group mean spot-forming units (SFU)±SD per 1×10⁵ CD4 T cells; ve, vehicle.

FIGS. 38A-38D depict activation of CD4 T cells only, as assessed by secretion of IFN-γ, IL-2 and TNF-α. IFN-γ+IL-2+ TNF-α secretion was assessed by a fluorospot assay after using MACS separation to isolate CD4⁺T cells (from pools of splenocytes from mice immunized with different formulated RNA constructs). Cells were then incubated with overlapping peptide pools covering the full length CSP protein (FIG. 38A) or controls (e.g., negative control: Trp1, 2 μg/mL (FIG. 38B); positive control: concanavalin A, 2 μg/mL (FIG. 38C); medium control (FIG. 38D)). Pooled samples were measured in triplicate and negative control was measured in duplicate; data points and bars represent the group mean spot-forming units (SFU)±SD per 1×10⁵ CD4 T cells; ve, vehicle.

FIGS. 39A-39D depict activation of CD8 T cells only, as assessed by secretion of IFN-γ. IFN-γ secretion was assessed by a fluorospot assay after using MACS separation to isolate CD8⁺T cells (from pools of splenocytes from mice immunized with different formulated RNA constructs). Cells were then incubated with overlapping peptide pools covering the full length CSP protein (FIG. 39A) or controls (e.g., negative control: Trp1, 2 μg/mL (FIG. 39B); positive control: concanavalin A, 2 μg/mL (FIG. 39C); medium control (FIG. 39D)). Pooled samples were measured in triplicate and negative control was measured in duplicate; data points and bars represent the group mean spot-forming units (SFU)±SD per 1×10⁵ CD8 T cells; ve, vehicle.

FIGS. 40A-40D depict activation of CD8 T cells only, as assessed by secretion of IL-2. IL-2 secretion was assessed by a fluorospot assay after using MACS separation to isolate CD8⁺T cells (from pools of splenocytes from mice immunized with different formulated RNA constructs). Cells were then incubated with overlapping peptide pools covering the full length CSP protein (FIG. 40A) or controls (e.g., negative control: Trp1, 2 μg/mL (FIG. 40B); positive control: concanavalin A, 2 g/mL (FIG. 40C); medium control (FIG. 40D)). Pooled samples were measured in triplicate and negative control was measured in duplicate; data points and bars represent the group mean spot-forming units (SFU)±SD per 1×10⁵ CD8 T cells; ve, vehicle.

FIGS. 41A-41D depict activation of CD8 T cells only, as assessed by secretion of TNF-α. TNF-α secretion was assessed by a fluorospot assay after using MACS separation to isolate CD8⁺T cells (from pools of splenocytes from mice immunized with different formulated RNA constructs). Cells were then incubated with overlapping peptide pools covering the full length CSP protein (FIG. 41A) or controls (e.g., negative control: Trp1, 2 μg/mL (FIG. 41B); positive control: concanavalin A, 2 μg/mL (FIG. 41C); medium control (FIG. 41D)). Pooled samples were measured in triplicate and negative control was measured in duplicate; data points and bars represent the group mean spot-forming units (SFU)±SD per 1×10⁵ CD8 T cells; ve, vehicle.

FIGS. 42A-42D depict activation of CD8 T cells only, as assessed by secretion of both IFN-γ and IL-2. IFN-γ+IL-2 secretion was assessed by a fluorospot assay after using MACS separation to isolate CD8⁺T cells (from pools of splenocytes from mice immunized with different formulated RNA constructs). Cells were then incubated with overlapping peptide pools covering the full length CSP protein (FIG. 42A) or controls (e.g., negative control: Trp1, 2 μg/mL (FIG. 42B); positive control: concanavalin A, 2 μg/mL FIG. 42C; medium control (FIG. 42D)). Pooled samples were measured in triplicate and negative control was measured in duplicate; data points and bars represent the group mean spot-forming units (SFU)±SD per 1×10⁵ CD8 T cells; ve, vehicle.

FIGS. 43A-43D depict activation of CD8 T cells only, as assessed by secretion of both IFN-γ and TNF-α. IFN-γ+ TNF-α secretion was assessed by a fluorospot assay after using MACS separation to isolate CD8⁺T cells (from pools of splenocytes from mice immunized with different formulated RNA constructs). Cells were then incubated with overlapping peptide pools covering the full length CSP protein (FIG. 43A) or controls (e.g., negative control: Trp1, 2 g/mL (FIG. 43B); positive control: concanavalin A, 2 μg/mL (FIG. 43C); medium control (FIG. 43D)). Pooled samples were measured in triplicate and negative control was measured in duplicate; data points and bars represent the group mean spot-forming units (SFU)±SD per 1×10⁵ CD8 T cells; ve, vehicle.

FIGS. 44A-44D depict activation of CD8 T cells only, as assessed by secretion of both IL-2 and TNF-α. IL-2+ TNF-α secretion was assessed by a fluorospot assay after using MACS separation to isolate CD8⁺T cells (from pools of splenocytes from mice immunized with different formulated RNA constructs). Cells were then incubated with overlapping peptide pools covering the full length CSP protein (FIG. 44A) or controls (e.g., negative control: Trp1, 2 μg/mL (FIG. 44B); positive control: concanavalin A, 2 μg/mL (FIG. 44C); medium control (FIG. 44D)). Pooled samples were measured in triplicate and negative control was measured in duplicate; data points and bars represent the group mean spot-forming units (SFU)±SD per 1×10⁵ CD8 T cells; ve, vehicle.

FIGS. 45A-45D depict activation of CD8 T cells only, as assessed by secretion of IFN-γ,IL-2 and TNF-α. IFN-γ+IL-2+ TNF-α secretion was assessed by a fluorospot assay after using MACS separation to isolate CD8⁺T cells (from pools of splenocytes from mice immunized with different formulated RNA constructs). Cells were then incubated with overlapping peptide pools covering the full length CSP protein (FIG. 45A) or controls (e.g., negative control: Trp1, 2 g/mL (FIG. 45B); positive control: concanavalin A, 2 μg/mL (FIG. 45C); medium control (FIG. 45D)). Pooled samples were measured in triplicate and negative control was measured in duplicate; data points and bars represent the group mean spot-forming units (SFU)±SD per 1×10⁵ CD8 T cells; ve, vehicle.

FIGS. 46A-46D depict protection of mice immunized with formulated RNA constructs against a challenge with PfCSP-expressing P. berghei sporozoites as well as immunogenicity induced by this immunization. FIG. 46A depicts percentage of protected mice up to 11 days after challenge with PfCSP-expressing P. berghei sporozoites, for mice immunized with formulated RNA constructs, vehicle, or positive control. Mice that received 100 μg of the 2A10 monoclonal antibody 24 h before the challenge were used as positive control. FIGS. 46B and 46C depict endpoint titers against full length PfCSP two weeks after the boost (day 35, FIG. 46B) and one day before the challenge (day 49, FIG. 46C) for mice immunized with formulated RNA constructs and mice injected with the vehicle only. Mean±SEM and individual animal values are shown. FIG. 46D shows a visual summary of binding of antibodies (generated by immunization with RNA constructs 2, 23 and 39 during challenge studies) to specific PfCSP epitopes.

FIGS. 47A-47E depict assessment of antibodies generated from mice immunized with a formulated RNA construct for ability to recognize native PfCSP on sporozoites and inhibit sporozoite viability and motility. FIG. 47A shows log of endpoint titers using fixed PfCSP-expressing P. berghei sporozoites. Symbols represent the mean±SEM using serum from individual mice. FIG. 47B shows estimated length of the circumsporozoite precipitation reaction (CSPR) elicited by serum samples from immunized mice as measured by flow cytometry (Forward Scatter Width (FSC-W)). Symbols represent the mean±SEM using serum from individual mice. FIG. 47C shows cytotoxicity of serum samples from immunized mice against sporozoites in suspension (PBS). Symbols represent the mean±SEM using serum from individual mice. FIG. 47D shows cytotoxicity in 3D (Matrigel). Symbols represent the mean±SEM using serum from individual mice. FIG. 47E shows inhibition of sporozoite gliding speed. Circles represent the mean±SEM of duplicates of pooled serum samples from each group.

FIGS. 48A-48B depict protection of mice immunized with formulated RNA constructs against a challenge with PfCSP-expressing P. berghei sporozoites as well as immunogenicity induced by this immunization from three separate experiments. FIG. 48A depicts percentage of protected mice up to 11 days after challenge with PfCSP-expressing P. berghei sporozoites, for mice immunized with formulated RNA constructs, vehicle (saline), or positive control. Mice that received 100 μg of the 2A10 monoclonal antibody 24 h before the challenge or Mosquirix® were used as positive control. FIG. 48B depicts endpoint titers against full length PfCSP two weeks after the boost (day 35) and one day before the challenge (day 49) for mice immunized with formulated RNA constructs and mice injected with the vehicle only. Mean±SEM and individual animal values are shown. Mice that received 100 μg of the 2A10 monoclonal antibody 24 h before the challenge were used as positive control in Experiment 1. Serum samples from this group were collected 20 h after the passive immunization with 2A₁₀. Mice immunized twice IM with 5 μg of Mosquirix® were used as positive control in Experiments 2 and 3. Mice injected with the vehicle were used as negative controls in all experiments.

FIGS. 49A-49E depict assessments of antibodies generated from mice immunized with formulated RNA constructs. FIG. 49A depicts assessment of antibodies generated from mice immunized with formulated RNA constructs for ability to recognize native PfCSP sporozoites. Depicted graph corresponds to Experiment 1 of challenge studies and shows log of anti-sporozoite endpoint titers using fixed PfCSP-expressing P. berghei sporozoites. Symbols represent the mean±SEM using serum from individual mice. 2A₁₀, positive antibody control; IFA, Immunofluorescence assay. FIG. 49B depicts assessment of antibodies generated from mice immunized with formulated RNA constructs for ability to inhibit sporozoite gliding motility. Depicted graph corresponds to Experiment 1 of challenge studies and shows sporozoite gliding speed (μm/s). Symbols represent the mean±SEM of duplicates of pooled serum samples from each group. 2A₁₀, positive antibody control; Veh, vehicle. FIG. 49C depicts assessment of antibodies generated from mice immunized with formulated RNA constructs for ability to bind and crosslink native PfCSP on the sporozoite surface. Depicted graph corresponds to Experiment 1 of challenge studies and represent the estimated length of the circumsporozoite Precipitation Reaction (CSPR) elicited by 17% immune sera as measured by flow cytometry (Forward Scatter Width (FSC-W)). Symbols represent the mean±SEM using serum from individual mice. 2A₁₀, positive antibody control; Veh, vehicle. FIG. 49D-49E show cytotoxicity of the antibodies present in serum samples from immunized mice against sporozoites in suspension (PBS), above, and 3D (Matrigel), below. FIG. 49D depicts the cytotoxicity of 17% immune sera measured against PfCSP-expressing P. berghei sporozoites in suspension and is presented as percentage of viable sporozoites. FIG. 49E depicts cytotoxicity of 17% immune sera against PfCSP-expressing P. berghei sporozoites measured in a 3D Matrigel and normalized to saline group (viability=100%). Symbols represent the mean±SEM using serum from individual mice. 2A₁₀, positive antibody control; Veh, vehicle; PBS, phosphate buffer saline.

FIG. 50 depicts assessment of antibodies generated from mice immunized with formulated RNA of 5 priority constructs for ability to recognize native PfCSP sporozoites. Each graph represents a different experiment and shows log of anti-sporozoite endpoint titers using fixed PfCSP-expressing P. berghei sporozoites. Bars represent the mean±SEM using serum from individual mice. 2A₁₀, positive antibody control; Mos, Mosquirix® positive control; IFA, Immunofluorescence assay.

FIG. 51 depicts assessment of antibodies generated from mice immunized with formulated RNA of 5 priority constructs for ability to inhibit sporozoite gliding motility. Each graph represents a different experiment and shows sporozoite gliding speed (μm/s). Bars represent the mean±SEM of duplicates (in Experiment 1) or single replicates (Experiments 2 and 3) of pooled serum samples from each group. 2A₁₀, positive antibody control; Mos, Mosquirix® positive control; Veh, vehicle.

FIG. 52 depicts assessment of antibodies generated from mice immunized with formulated RNA of 5 priority constructs for ability to bind and crosslink native PfCSP on the sporozoite surface. Each graph represents a different experiment and shows the estimated length of the circumsporozoite Precipitation Reaction (CSPR) elicited by 17% immune sera as measured by flow cytometry (Forward Scatter Width (FSC-W)). Symbols represent the mean±SEM using serum from individual mice. 2A₁₀, positive antibody control; Mos, Mosquirix® positive control; Veh, vehicle.

FIGS. 53A-53B show cytotoxicity of the antibodies present in serum samples from mice immunized with 5 priority formulated RNA constructs against sporozoites in suspension (PBS), above, and 3D (Matrigel), below. FIG. 53A depicts the cytotoxicity of 17% immune sera measured against PfCSP-expressing P. berghei sporozoites in suspension and is presented as percentage of viable sporozoites. FIG. 53B depicts cytotoxicity of 17% immune sera against PfCSP-expressing P. berghei sporozoites measured in a 3D Matrigel and normalized to saline group (viability=100%). Each graph represents an independent challenge experiment (designated Experiment 1, Experiment 2 and Experiment 3). Bars represent the mean±SEM using serum from individual mice. 2A₁₀, positive antibody control; Mosquirix® positive control; Veh, vehicle; PBS, phosphate buffer saline.

FIG. 54 includes schematics of exemplary Plasmodium polypeptide constructs. The circle with RNA construct 91 indicates a T337N mutation to remove an O-fucose site, as numbered according to SEQ ID NO: 1.

### DEFINITIONS

Compounds of this disclosure include those described generally above and are further illustrated by the classes, subclasses, and species disclosed herein. As used herein, the following definitions shall apply unless otherwise indicated. For purposes of this disclosure, the chemical elements are identified in accordance with the Periodic Table of Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents each of which are hereby incorporated by reference.

Unless otherwise stated, structures depicted herein are meant to include all stereoisomeric (e.g., enantiomeric or diastereomeric) forms of the structure, as well as all geometric or conformational isomeric forms of the structure. For example, the R and S configurations of each stereocenter are contemplated as part of the disclosure. Therefore, single stereochemical isomers, as well as enantiomeric, diastereomeric, and geometric (or conformational) mixtures of provided compounds are within the scope of the disclosure. For example, in some cases, provided compounds show one or more stereoisomers of a compound, and unless otherwise indicated, represents each stereoisomer alone and/or as a mixture. Unless otherwise stated, all tautomeric forms of provided compounds are within the scope of the disclosure.

Unless otherwise indicated, structures depicted herein are meant to include compounds that differ only in the presence of one or more isotopically enriched atoms. For example, compounds having the present structures including replacement of hydrogen by deuterium or tritium, or replacement of a carbon by 13C- or 14C-enriched carbon are within the scope of this disclosure.

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Agent: As used herein, the term “agent,” may refer to a physical entity. In some embodiments, an agent may be characterized by a particular feature and/or effect. For example, as used herein, the term “therapeutic agent” refers to a physical entity has a therapeutic effect and/or elicits a desired biological and/or pharmacological effect. In some embodiments, an agent may be a compound, molecule, or entity of any chemical class including, for example, a small molecule, polypeptide, nucleic acid, saccharide, lipid, metal, or a combination or complex thereof.

Amino acid: In its broadest sense, as used herein, the term “amino acid” refers to a compound and/or substance that can be, is, or has been incorporated into a polypeptide chain, e.g., through formation of one or more peptide bonds. In some embodiments, an amino acid has the general structure H₂N—C(H) (R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

Antigen: The term “antigen”, as used herein, refers to an agent that elicits an immune response; and/or an agent that binds to a T cell receptor (e.g., when presented by an MHC molecule) or to an antibody.

Anti-malaria Immune response: The term “anti-malaria immune response”, as used herein, refers to an immune response directed to one or more antigens derived from Plasmodium.

Associated: Two events or entities are “associated” with one another, as that term is used herein, if the presence, level, degree, type and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of, susceptibility to, severity of, stage of, etc. the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

C-terminal domain: The term “C-terminal domain”, as used herein, refers to a region of a CSP polypeptide that corresponds to amino acids 273-397 of wild-type CSP sequence of Plasmodium falciparum (isolate 3D7) (SEQ ID NO:1).

C-terminal region: The term “C-terminal region”, as used herein, refers to a region of a CSP polypeptide that corresponds to amino acids 273-375 of wild-type CSP sequence (SEQ ID NO:1). In some embodiments, a serine follows immediately after the C-terminal region. In some embodiments, a serine and a valine follow immediately after the C-terminal region.

Central domain: The term “central domain”, as used herein, refers to a region of a CSP polypeptide that corresponds to amino acids 105-272 of wild-type CSP sequence (SEQ ID NO:1).

Characteristic portion: As used herein, the term “characteristic portion”, in the broadest sense, refers to a portion of a polypeptide or region thereof whose presence (or absence) correlates with presence (or absence) of a particular feature, attribute, or activity of the polypeptide or region thereof. In some embodiments, a characteristic portion of a polypeptide or region thereof is a portion that is found in the polypeptide or region thereof and in related polypeptide or region thereof that share the particular feature, attribute or activity, but not in those that do not share the particular feature, attribute or activity. In certain embodiments, a characteristic portion shares at least one functional characteristic with the intact polypeptide or region thereof. For example, in some embodiments, a “characteristic portion” of a polypeptide or region thereof is one that contains a continuous stretch of amino acids, or a collection of continuous stretches of amino acids, that together are characteristic of the polypeptide or region thereof. In some embodiments, each such continuous stretch generally contains at least 2, 5, 10, 15, 20, 50, or more amino acids. In general, a characteristic portion of a polypeptide or region thereof (e.g., CSP, its N terminal domain, its major repeat region etc.) is one that, in addition to the sequence and/or structural identity specified above, shares at least one functional characteristic with the relevant intact polypeptide or region thereof. In some embodiments, a characteristic portion may be biologically active. In some embodiments, a fragment as described herein can be a portion. Accordingly, in some embodiments, a characteristic fragment can be a “characteristic portion.”

Combination therapy: As used herein, the term “combination therapy” refers to those situations in which a subject is simultaneously exposed to two or more therapeutic regimens (e.g., two or more therapeutic agents (e.g., two or more antibody agents)). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, administration of combination therapy may involve administration of one or more agent(s) or modality (ies) to a subject receiving the other agent(s) or modality (ies) in the combination. For clarity, combination therapy does not require that individual agents be administered together in a single composition (or even necessarily at the same time), although in some embodiments, two or more agents, or active moieties thereof, may be administered together in a combination composition.

Comparable: As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Corresponding to: As used herein, the term “corresponding to” refers to a relationship between two or more entities. For example, the term “corresponding to” may be used to designate the position/identity of a structural element in a compound or composition relative to another compound or composition (e.g., to an appropriate reference compound or composition). For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of ordinary skill will appreciate that, for purposes of simplicity, residues in a polypeptide are often designated using a canonical numbering system based on a reference related polypeptide, so that an amino acid “corresponding to” a residue at position 190, for example, need not actually be the 190th amino acid in a particular amino acid chain but rather corresponds to the residue found at 190 in the reference polypeptide; those of ordinary skill in the art readily appreciate how to identify “corresponding” amino acids. For example, those skilled in the art will be aware of various sequence alignment strategies, including software programs such as, for example, BLAST, CS-BLAST, CUSASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE that can be utilized, for example, to identify “corresponding” residues in polypeptides and/or nucleic acids in accordance with the present disclosure. Those of skill in the art will also appreciate that, in some instances, the term “corresponding to” may be used to describe an event or entity that shares a relevant similarity with another event or entity (e.g., an appropriate reference event or entity). To give but one example, a gene or protein in one organism may be described as “corresponding to” a gene or protein from another organism in order to indicate, in some embodiments, that it plays an analogous role or performs an analogous function and/or that it shows a particular degree of sequence identity or homology, or shares a particular characteristic sequence element.

Dosing regimen: Those skilled in the art will appreciate that the term “dosing regimen” (or “therapeutic regimen”) may be used to refer to a set of unit doses (typically more than one) that are administered individually to a subject, typically separated by periods of time. In some embodiments, a given therapeutic agent has a recommended dosing regimen, which may involve one or more doses.

Encode: As used herein, the term “encode” or “encoding” refers to sequence information of a first molecule that guides production of a second molecule having a defined sequence of nucleotides (e.g., a polyribonucleotide) or a defined sequence of amino acids. For example, a DNA molecule can encode an RNA molecule (e.g., by a transcription process that includes a DNA-dependent RNA polymerase enzyme). An RNA molecule can encode a polypeptide (e.g., by a translation process). Thus, a gene, a cDNA, or an RNA molecule encodes a polypeptide if transcription and translation of RNA corresponding to that gene produces the polypeptide in a cell or other biological system. In some embodiments, a coding region of a polyribonucleotide encoding a target antigen refers to a coding strand, the nucleotide sequence of which is identical to the polyribonucleotide sequence of such a target antigen. In some embodiments, a coding region of a polyribonucleotide encoding a target antigen refers to a non-coding strand of such a target antigen, which may be used as a template for transcription of a gene or cDNA.

Expression: As used herein, the term “expression” of a nucleic acid sequence refers to the generation of a gene product from the nucleic acid sequence. In some embodiments, a gene product can be a transcript, e.g., a polyribonucleotide as provided herein. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, etc.); (3) translation of an RNA into a polypeptide or protein; and/or (4) post-translational modification of a polypeptide or protein.

Helper antigen: As used herein, the term “helper antigen” refers to an antigen that is included in a polypeptide comprising one or more CSP polypeptide regions or portion thereof, where the antigen is not derived from a CSP polypeptide.

Heterologous: As used herein, the term “heterologous”, with respect to secretory signal or transmembrane region, refers to a secretory signal or transmembrane region from a virus or an organism other than Plasmodium.

Homology: As used herein, the term “homology” or “homolog” refers to the overall relatedness between polynucleotide molecules (e.g., DNA molecules and/or RNA molecules) and/or between polypeptide molecules. In some embodiments, polynucleotide molecules (e.g., DNA molecules and/or RNA molecules) and/or polypeptide molecules are considered to be “homologous” to one another if their sequences are at least 15%, 20%, 25%, 30%, 35%, 40%, 45%, 50%, 55%, 60%, 65%, 70%, 75%, 80%, 85%, 90%, 95%, or 99% identical. In some embodiments, polynucleotide molecules (e.g., DNA molecules and/or RNA molecules) and/or polypeptide molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar (e.g., containing residues with related chemical properties at corresponding positions). For example, as is well known by those of ordinary skill in the art, certain amino acids are typically classified as similar to one another as “hydrophobic” or “hydrophilic” amino acids, and/or as having “polar” or “non-polar” side chains. Substitution of one amino acid for another of the same type may often be considered a “homologous” substitution.

Identity: As used herein, the term “identity” refers to the overall relatedness between polynucleotide molecules (e.g., DNA molecules and/or RNA molecules) and/or between polypeptide molecules. In some embodiments, polynucleotide molecules (e.g., DNA molecules and/or RNA molecules) and/or between polypeptide molecules are considered to be “substantially identical” to one another if their sequences are at least 80%, 85%, 90%, 95%, 96%, 97%, 98%, or 99% identical. Calculation of the percent identity of two nucleic acid or polypeptide sequences, for example, can be performed by aligning the two sequences for optimal comparison purposes (e.g., gaps can be introduced in one or both of a first and a second sequence for optimal alignment and non-identical sequences can be disregarded for comparison purposes). In certain embodiments, the length of a sequence aligned for comparison purposes is at least 30%, at least 40%, at least 50%, at least 60%, at least 70%, at least 75%, at least 80%, at least 85%, at least 90%, at least 95%, at least 96%, at least 97%, at least 98%, at least 99%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller, 1989, which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Increased, Induced, or Reduced: As used herein, these terms or grammatically comparable comparative terms, indicate values that are relative to a comparable reference measurement. For example, in some embodiments, an assessed value achieved with a provided composition (e.g., a pharmaceutical composition) may be “increased” relative to that obtained with a comparable reference composition. Alternatively or additionally, in some embodiments, an assessed value achieved in a subject may be “increased” relative to that obtained in the same subject under different conditions (e.g., prior to or after an event; or presence or absence of an event such as administration of a composition (e.g., a pharmaceutical composition) as described herein, or in a different, comparable subject (e.g., in a comparable subject that differs from the subject of interest in prior exposure to a condition, e.g., absence of administration of a composition (e.g., a pharmaceutical composition) as described herein). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance. In some embodiments, the term “reduced” or equivalent terms refers to a reduction in the level of an assessed value by at least 5%, at least 10%, at least 20%, at least 50%, at least 75% or higher, as compared to a comparable reference. In some embodiments, the term “reduced” or equivalent terms refers to a complete or essentially complete inhibition, i.e., a reduction to zero or essentially to zero. In some embodiments, the term “increased” or “induced” refers to an increase in the level of an assessed value by at least 10%, at least 20%, at least 30%, at least 40%, at least 50%, at least 80%, at least 100%, at least 200%, at least 500%, or higher, as compared to a comparable reference.

In order: As used herein with reference to a polynucleotide or polyribonucleotide, “in order” refers to the order of features from 5′ to 3′ along the polynucleotide or polyribonucleotide. As used herein with reference to a polypeptide, “in order” refers to the order of features moving from the N-terminal-most of the features to the C-terminal-most of the features along the polypeptide. “In order” does not mean that no additional features can be present among the listed features. For example, if Features A, B, and C of a polynucleotide are described herein as being “in order, Feature A, Feature B, and Feature C,” this description does not exclude, e.g., Feature D being located between Features A and B.

Isolated: The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

Junction: The term “junction”, as used herein, refers to a region of a CSP polypeptide that corresponds to amino acids 98-104 of wild-type CSP sequence (SEQ ID NO: 1).

Junction region: The term “junction region”, as used herein, refers to a region of a CSP polypeptide that corresponds to amino acids 93-104 of wild-type CSP sequence (SEQ ID NO:1). Junction region variant: The term “junction region variant”, as used herein, refers to a junction region that comprises one or more substitution mutation as compared to amino acids 93-104 of wild-type CSP sequence (SEQ ID NO:1).

Linker. As used herein, the term “linker” refers to a portion of a polypeptide that connects different regions, portions, or antigens to one another.

Lipld: As used herein, the terms “lipid” and “lipid-like material” are broadly defined as molecules which comprise one or more hydrophobic moieties or groups and optionally also one or more hydrophilic moieties or groups. Molecules comprising hydrophobic moieties and hydrophilic moieties are also typically denoted as amphiphiles.

Major repeat region: As used herein, the term “major repeat region” refers to a region of a CSP polypeptide that corresponds to amino acids 129-272 of wild-type CSP sequence (SEQ ID NO:1) and contains 35 repeats of the amino acid sequence NANP (SEQ ID NO: 147). The 35 repeats of the amino acid sequence NANP (SEQ ID NO: 147) are separated into two contiguous stretches, the first stretch containing 17 repeats of the amino acid sequence NANP (SEQ ID NO: 147) and second stretch containing 18 repeats of the amino acid sequence NANP (SEQ ID NO: 147) which flank an amino acid sequence of NVDP (SEQ ID NO: 144). A portion of the major repeat region contains at least the amino acid sequence NPNA (SEQ ID NO: 141). Preferably a portion of the major repeat region contains at least the amino acid sequences NANPNA (SEQ ID NO: 153) and NPNANP (SEQ ID NO: 150). As used herein, “repeat” in reference to sequence A refers to sequence A being present once, and “one or more repeats” of sequence A refers to sequence A being present one or more times.

Merozoite stage specific Plasmodium antigen: As used herein, the term “merozoite stage specific Plasmodium antigen” refers to an antigen that is expressed during the merozoite stage of the Plasmodium life cycle.

Minor repeat region: As used herein, the term “minor repeat region” refers to a region of a CSP polypeptide that corresponds to amino acids 105-128 of wild-type CSP sequence (SEQ ID NO:1) and contains 3 repeats of the amino acid sequence NANPNVDP (SEQ ID NO: 102). A minor repeat region does not contain the amino acid sequence NPNA (SEQ ID NO: 141), and does not contain the amino acid sequence NANPNA (SEQ ID NO: 153) or NPNANP (SEQ ID NO: 150). As used herein, “repeat” in reference to sequence A refers to sequence A being present once, and three repeats of sequence A refers to sequence A being present three times.

Multimerization region: As used herein, the term “multimerization region” refers to a region that directs assembly of multimers into a complex, where each multimer comprises a polypeptide associated with the multimerization region.

N-terminal domain: As used herein, the term “N-terminal domain” refers to a region of a CSP polypeptide that corresponds to amino acids 19-92 of wild-type CSP sequence (SEQ ID NO:1).

N-terminal end region: As used herein, the term “N-terminal end region” refers to a region of a CSP polypeptide that corresponds to amino acids 81-92 of wild-type CSP sequence (SEQ ID NO:1).

N-terminal region: As used herein, the term “N-terminal region” refers to a region of a CSP polypeptide that corresponds to amino acids 19-80 of wild-type CSP sequence (SEQ ID NO:1).

RNA lipid nanoparticle: As used herein, the term “RNA lipid nanoparticle” refers to a nanoparticle comprising at least one lipid and RNA molecule(s), e.g., one or more polyribonucleotides as provided herein. In some embodiments, an RNA lipid nanoparticle comprises at least one cationic amino lipid. In some embodiments, an RNA lipid nanoparticle comprises at least one cationic amino lipid, at least one helper lipid, and at least one polymer-conjugated lipid (e.g., PEG-conjugated lipid). In various embodiments, RNA lipid nanoparticles as described herein can have an average size (e.g., Z-average) of about 100 nm to 1000 nm, or about 200 nm to 900 nm, or about 200 nm to 800 nm, or about 250 nm to about 700 nm. In some embodiments of the present disclosure, RNA lipid nanoparticles can have a particle size (e.g., Z-average) of about 30 nm to about 200 nm, or about 30 nm to about 150 nm, about 40 nm to about 150 nm, about 50 nm to about 150 nm, about 60 nm to about 130 nm, about 70 nm to about 110 nm, about 70 nm to about 100 nm, about 80 nm to about 100 nm, about 90 nm to about 100 nm, about 70 to about 90 nm, about 80 nm to about 90 nm, or about 70 nm to about 80 nm. In some embodiments, an average size of lipid nanoparticles is determined by measuring the average particle diameter. In some embodiments, RNA lipid nanoparticles may be prepared by mixing lipids with RNA molecules described herein.

Neutralization: As used herein, the term “neutralization” refers to an event in which binding agents such as antibodies bind to a biological active site of a parasite such as a receptor binding protein, thereby inhibiting the parasitic infection of cells. In some embodiments, the term “neutralization” refers to an event in which binding agents eliminate or significantly reduce ability of infecting cells.

Nucleic acid/Polynucleotide: As used herein, the term “nucleic acid” refers to a polymer of at least 10 nucleotides or more. In some embodiments, a nucleic acid is or comprises DNA. In some embodiments, a nucleic acid is or comprises RNA. In some embodiments, a nucleic acid is or comprises peptide nucleic acid (PNA). In some embodiments, a nucleic acid is or comprises a single stranded nucleic acid. In some embodiments, a nucleic acid is or comprises a double-stranded nucleic acid. In some embodiments, a nucleic acid comprises both single and double-stranded portions. In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid”. In some embodiments, a nucleic acid comprises one or more, or all, natural residues (e.g., adenine, cytosine, deoxyadenosine, deoxycytidine, deoxyguanosine, deoxythymidine, guanine, thymine, uracil). In some embodiments, a nucleic acid comprises on or more, or all, non-natural residues. In some embodiments, a non-natural residue comprises a nucleoside analog (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 6—O-methylguanine, 2-thiocytidine, methylated bases, intercalated bases, and combinations thereof). In some embodiments, a non-natural residue comprises one or more modified sugars (e.g., 2′-fluororibose, ribose, 2′-deoxyribose, arabinose, and hexose) as compared to those in natural residues. In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro), reproduction in a recombinant cell or system, or chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, or 20,000 or more residues or nucleotides long.

Pharmaceutically effective amount: The term “pharmaceutically effective amount” or “therapeutically effective amount” refers to the amount which achieves a desired reaction or a desired effect alone or together with further doses. In the case of the treatment of a particular disease (e.g., malaria), a desired reaction in some embodiments relates to inhibition of the course of the disease (e.g., malaria). In some embodiments, such inhibition may comprise slowing down the progress of a disease (e.g., malaria) and/or interrupting or reversing the progress of the disease (e.g., malaria). In some embodiments, a desired reaction in a treatment of a disease (e.g., malaria) may be or comprise delay or prevention of the onset of a disease (e.g., malaria) or a condition (e.g., a malaria associated condition). An effective amount of a composition (e.g., a pharmaceutical composition) described herein will depend, for example, on disease (e.g., malaria) or a condition (e.g., a malaria associated condition) to be treated, the severity of such a disease (e.g., malaria) or a condition (e.g., a malaria associated condition), individual parameters of the patient, including, e.g., age, physiological condition, size and weight, the duration of treatment, the type of an accompanying therapy (if present), the specific route of administration and similar factors. Accordingly, doses of a composition (e.g., a pharmaceutical composition) described herein may depend on various of such parameters. In the case that a reaction in a patient is insufficient with an initial dose, higher doses (or effectively higher doses achieved by a different, more localized route of administration) may be used.

Polypeptide: As used herein, the term “polypeptide” refers to a polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications comprise acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 35 or more amino acids; in some embodiments, a conserved region encompasses at least one stretch of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more contiguous amino acids. In some embodiments, a relevant polypeptide may comprise or consist of a fragment of a parent polypeptide. In some embodiments, a polypeptide is a Plasmodium polypeptide construct described herein. A Plasmodium polypeptide construct is a polypeptide that includes one or more Plasmodium proteins, or one or more portions thereof. In some embodiments, a Plasmodium polypeptide construct described herein includes at least one region of Plasmodium CSP or a portion thereof. In some embodiments, a Plasmodium polypeptide construct additionally includes one or more additional amino acid sequences, such as a secretory signal (e.g., a heterologous secretory signal), a transmembrane region (e.g., a heterologous transmembrane region), a helper antigen, a multimerization region, and/or a linker, as described herein.

Prevent: As used herein, the term “prevent” or “prevention” when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time. In some embodiments, prevention refers to reducing the risk of developing clinical malaria.

R1: The term “R1”, as used herein, refers to a region of a CSP polypeptide that corresponds to amino acids 93-97 of wild-type CSP sequence (SEQ ID NO: 1).

Reference: As used herein, the term “reference” describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Ribonucleic acid (RNA) or Polyribonucleotide: As used herein, the term “ribonucleic acid,” “RNA,” or “polyribonucleotide” refers to a polymer of ribonucleotides. In some embodiments, an RNA is single stranded. In some embodiments, an RNA is double stranded. In some embodiments, an RNA comprises both single and double stranded portions. In some embodiments, an RNA can comprise a backbone structure as described in the definition of “Nucleic acid/Polynucleotide” above. An RNA can be a regulatory RNA (e.g., siRNA, microRNA, etc.), or a messenger RNA (mRNA). In some embodiments, an RNA is an mRNA. In some embodiments, where an RNA is a mRNA, a RNA typically comprises at its 3′ end a poly(A) region. In some embodiments, where an RNA is a mRNA, an RNA typically comprises at its 5′ end an art-recognized cap structure, e.g., for recognizing and attachment of a mRNA to a ribosome to initiate translation. In some embodiments, a RNA is a synthetic RNA. Synthetic RNAs include RNAs that are synthesized in vitro (e.g., by enzymatic synthesis methods and/or by chemical synthesis methods). In some embodiments, a polyribonucleotide encodes a polypeptide, which is preferably is a Plasmodium polypeptide construct.

Ribonucleotide: As used herein, the term “ribonucleotide” encompasses unmodified ribonucleotides and modified ribonucleotides. For example, unmodified ribonucleotides include the purine bases adenine (A) and guanine (G), and the pyrimidine bases cytosine (C) and uracil (U). Modified ribonucleotides may include one or more modifications including, but not limited to, for example, (a) end modifications, e.g., 5′ end modifications (e.g., phosphorylation, dephosphorylation, conjugation, inverted linkages, etc.), 3′ end modifications (e.g., conjugation, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, and (d) internucleoside linkage modifications, including modification or replacement of the phosphodiester linkages. The term “ribonucleotide” also encompasses ribonucleotide triphosphates including modified and non-modified ribonucleotide triphosphates.

Secretory signal: As used herein, the term “secretory signal” refers to an amino acid sequence motif that targets associated polypeptides for translocation to a secretory pathway.

Subject: As used herein, the term “subject” refers to an organism to be administered with a composition described herein, e.g., for experimental, diagnostic, prophylactic, and/or therapeutic purposes. Typical subjects include animals (e.g., mammals such as mice, rats, rabbits, non-human primates, domestic pets, etc.) and humans. In preferred embodiments, a subject is a human subject. In some embodiments, a subject is suffering from a disease, disorder, or condition (e.g., malaria and/or a malaria-associated condition). In some embodiments, a subject is susceptible to a disease, disorder, or condition (e.g., malaria and/or a malaria-associated condition). In some embodiments, a subject displays one or more symptoms or characteristics of a disease, disorder, or condition (e.g., malaria and/or a malaria-associated condition). In some embodiments, a subject displays one or more non-specific symptoms of a disease, disorder, or condition (e.g., malaria and/or a malaria-associated condition). In some embodiments, a subject does not display any symptom or characteristic of a disease, disorder, or condition (e.g., malaria and/or a malaria-associated condition). In some embodiments, a subject is someone with one or more features characteristic of susceptibility to or risk of a disease, disorder, or condition (e.g., malaria and/or a malaria-associated condition). In some embodiments, a subject is a patient. In some embodiments, a subject is an individual to whom diagnosis and/or therapy is and/or has been administered.

Suffering from: An individual who is “suffering from” a disease, disorder, and/or condition (e.g., malaria and/or a malaria-associated condition) has been diagnosed with and/or displays one or more symptoms of a disease, disorder, and/or condition.

Susceptible to: An individual who is “susceptible to” a disease, disorder, and/or condition (e.g., malaria and/or a malaria-associated condition) is one who has a higher risk of developing the disease, disorder, and/or condition (e.g., malaria and/or a malaria-associated condition) than does a member of the general public. In some embodiments, an individual who is susceptible to a disease, disorder and/or condition (e.g., malaria and/or a malaria-associated condition) may not have been diagnosed with the disease, disorder, and/or condition (e.g., malaria and/or a malaria-associated condition). In some embodiments, an individual who is susceptible to a disease, disorder, and/or condition (e.g., malaria and/or a malaria-associated condition) may exhibit symptoms of the disease, disorder, and/or condition (e.g., malaria and/or a malaria-associated condition). In some embodiments, an individual who is susceptible to a disease, disorder, and/or condition (e.g., malaria and/or a malaria-associated condition) may not exhibit symptoms of the disease, disorder, and/or condition (e.g., malaria and/or a malaria-associated condition). In some embodiments, an individual who is susceptible to a disease, disorder, and/or condition (e.g., malaria and/or a malaria-associated condition) will develop the disease, disorder, and/or condition (e.g., malaria and/or a malaria-associated condition). In some embodiments, an individual who is susceptible to a disease, disorder, and/or condition (e.g., malaria and/or a malaria-associated condition) will not develop the disease, disorder, and/or condition (e.g., malaria and/or a malaria-associated condition).

Therapy: The term “therapy” refers to an administration or delivery of an agent or intervention that has a therapeutic effect and/or elicits a desired biological and/or pharmacological effect (e.g., has been demonstrated to be statistically likely to have such effect when administered to a relevant population). In some embodiments, a therapeutic agent or therapy is any substance that can be used to alleviate, ameliorate, relieve, inhibit, prevent, delay onset of, reduce severity of, and/or reduce incidence of one or more symptoms or features of a disease, disorder, and/or condition (e.g., malaria and/or a malaria-associated condition). In some embodiments, a therapeutic agent or therapy is a medical intervention that can be performed to alleviate, relieve, inhibit, present, delay onset of, reduce severity of, and/or reduce incidence of one or more symptoms or features of a disease, disorder, and/or condition.

Transmembrane region: As used herein, the term “transmembrane region” refers to a region of a polypeptide that spans a biological membrane, such as the plasma membrane of a cell.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition (e.g., malaria and/or a malaria-associated condition). Treatment may be administered to a subject who does not exhibit signs of a disease, disorder, and/or condition (e.g., malaria and/or a malaria-associated condition). In some embodiments, treatment may be administered to a subject who exhibits only early signs of the disease, disorder, and/or condition (e.g., malaria and/or a malaria-associated condition), for example for the purpose of decreasing the risk of developing pathology associated with the disease, disorder, and/or condition. In some embodiments, treatment may be administered to a subject at a later-stage of disease, disorder, and/or condition (e.g., malaria and/or a malaria-associated condition).

Variant: As used herein, the term “variant” refers to a molecule that shows significant structural (e.g., primary or secondary) identity with a reference molecule but differs structurally from the reference molecule. For example, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone).

### DETAILED DESCRIPTION OF CERTAIN EMBODIMENTS

### I. Malaria

Malaria is a mosquito-borne infectious disease caused by single-celled eukaryotic Plasmodium parasites that are transmitted by the bite of Anopheles spp. mosquitoes (Phillips, M., et al. Malaria. Nat Rev Dis Primers 3, 17050, 2017, which is incorporated herein by reference in its entirety). Mosquitoes that transmit malaria must have been infected through a previous blood meal taken from an infected subject (e.g., a human). When a mosquito bites an infected subject a small amount of blood is taken in containing Plasmodium parasites. The infected mosquito can then subsequently bite a non-infected subject, infecting the subject.

Malaria remains one of the most serious infectious diseases, causing approximately 200 million clinical cases and 500,000-600,000 deaths annually. Although significant effort has been invested in developing therapeutic treatments for malaria, many Plasmodium parasites have developed resistance to available therapeutics. According to Malaria Eradication Research Agenda Initiative, malaria eradication will only be achievable through effective vaccination.

In 2015, the European Medicines Agency gave a positive review to a malaria vaccine candidate known as “RTS,S”, a milestone in malaria vaccine development. In 2019, the World Health Organization launched pilot programs that provide RTS,S to children at least 5 months of age in parts of three sub-Saharan African countries. RTS,S/AS01 is an adjuvanted protein subunit vaccine that consists of a portion of the major repeat region and the C-terminus of CSP from Plasmodium falciparum fused to the Hepatitis B surface antigen (HBsAg). The vaccine is a mix of this PfCSP-HBsAg compound with HBsAg that forms virus-like particles (RTS,S/AS01; Mosquirix®). RTS,S is administered according to a regimen that requires four doses: an initial 3-dose schedule given at least 1 month apart, and a 4th dose 15-18 months after dose 3 (see, for example, Vandoolaeghe & Schuerman Expert Rev Vaccines. 15:1481, 2016; PATH_MVI_RTSS_Fact Sheet_042019, each of which is incorporated herein by reference in its entirety). Reports indicate that RTS,S protects approximately 30% to 50% of children from clinical disease over 18 months. RTS,S has been reported to induce protective antibody and CD4⁺T-cell responses, but only negligible CD8+ T cell responses (see, for example, Moris et al. Hum Vaccin Immunother 14:17, 2018, which is incorporated herein by reference in its entirety). Phase III studies of RTS,S delivered as a three-dose series with a booster after 1 yr (year) showed moderate vaccine efficacy in children aged 5 to 17 months preventing 36% of clinical malaria cases over the full study period with a median follow-up of 4 yrs, with a range of 20% in high to 66% in low transmission settings. Furthermore, published literature suggests that protection wanes over time including reports of potential negative efficacy after 5 yrs in children with high malaria exposure (Olotu et al. 2016, N. Engl. J. Med. 374:2519-29) which is incorporated herein by reference in its entirety). Thus, an effective malaria vaccine remains an unmet medical need of critical importance for global health.

### A. Lifecycle

During a blood meal, infected mosquitos inject, along with their anticoagulating saliva, sporozoites known as the liver stage of Plasmodium spp. Sporozoites journey through the skin to the lymphatics and into hepatocytes of the liver. This journey happens very quickly; it can be complete within only a few minutes (Sinnis et al., Parasitol Int. 2007 September; 56 (3): 171-8, which is incorporated herein by reference in its entirety). This is a time known to be a bottle kneck of Malaria infection most favorable for therapeutic intervention, as only a small number (thought to be a few hundred at maximum) of sporozoites are injected by the mosquito, with only fraction of that number establishing infection in the liver and developing into mature live-stage parasites (Flores-Garcia et al., mBio. 2018 Nov. 20;9 (6): e02194-18, which is incorporated herein by reference in its entirety). Thus, a subject whose immune system is primed to clear sporozoites before they enter hepatocytes can efficiently clear an infection.

One particular challenge associated with clearing a malarial infection during this bottle neck is that the most abundant and immunogenic protein on the sporozoite surface, the circumsporozoite protein (CSP), is only exposed to the immune system in small quantities and for short duration of time due to the variably low inoculum from the mosquito and the kinetics of hepatocyte infection after inoculation. After liver infection is established, the parasite differentiates into a stage which no longer expresses CSP and instead has a different mosaic of surface antigens. Furthermore, due to the density and close proximity of neighboring CSPs on the surface of the parasite coupled with the bi-valency of antibodies, binding of antibodies to CSP can produce a phenomenon referred to as CSP precipitation reaction, whereby antibodies can crosslink neighboring CSP and cause them to precipitate and shed from the parasite surface, leaving a trail of precipitated antibody bound CSP that the parasite can replace through its normal CSP translocation process (Livingstone et al., Sci Rep 11, 5318 (2021); Steward et al., J Protozool. 1991 July-August; 38 (4): 411-21, each of which is incorporated herein by reference in its entirety).

When moving from an inoculation site in the skin to the liver, sporozoites traverse host cells (Mota et al., Science 2001 Jan. 5; 291 (5501): 141-4). Sporozoites traverse different types of host cells at the dermis, including fibroblasts and phagocytes (Amino et al., Cell Host Microbe. 2008 Feb. 14; 3 (2): 88-96, which is incorporated herein by reference in its entirety), and the liver sinusoidal barrier, containing liver endothelial cells and Kupffer cells (Frevert et al., PLOS Biol 3 (6): e192. 2005, which is incorporated herein by reference in its entirety) and sinusoidal endothelial cells (Tavares et al., J Exp Med 2013 May 6; 210 (5): 905-15, which is incorporated herein by reference in its entirety), in order to gain access to hepatocytes. Sporozoites preferentially traverse cells with low-sulfated heparin sulfate proteoglycans (HSPGs) but preferentially invade cells with high-sulfated HSPGs (Coppi et al., Cell Host & Microbe 2, 316-327, November 2007, which is incorporated herein by reference in its entirety).

Cell traversal was first observed as non-phagocytic entry of P. berghei sporozoites into macrophages followed by “escape” from these cells (Vanderberg et al., J. Euk. Microbiol. 37:528-536, 1990, which is incorporated herein by reference in its entirety). The biochemical, biophysical, and stepwise processes of traversal are still being explored. However, it has been suggested by electron microscopy that host cell rupture occurs upon entry and exit from the host cell (Mota et al., 2001; Tavares et al., 2013, each of which is incorporated herein by reference in its entirety). It has also been shown that P. yoelii sporozoites can enter hepatocytes via a transient vacuole and that host membrane rupture occurs upon cell exit rather than cell entry (Risco-Castillo et al., Cell Host Microbe 2015 Nov. 11; 18 (5): 593-603, which is incorporated herein by reference in its entirety).

Sporozoites also traverse hepatocytes before establishing a productive hepatocyte infection (Mota et al., 2001, which is incorporated herein by reference in its entirety). Several possibilities emerged as to why this occurs. The first hypothesis suggested that migration through hepatocytes primes parasites for invasion by activating apical exocytosis (Mota et al., Nat Med 2002 November; 8 (11): 1318-22, which is incorporated herein by reference in its entirety). The second theory suggested that traversal releases hepatocyte growth factor (HGF), making neighboring hepatocytes more susceptible to infection (Carrolo et al., Nat Med. 2003 November; 9 (11): 1363-9, which is incorporated herein by reference in its entirety). Lastly, other studies suggest that it takes some time for sporozoites to switch off the machinery for traversal and activate invasion machinery (Amino et al., 2008, Coppi et al., 2007, each of which is incorporated herein by reference in its entirety), and that traversal primarily functions to penetrate cell barriers and avoid phagocytosis en route to the liver (Amino et al., 2008, Coppi et al., 2007, Tavares et al., 2013, each of which is incorporated herein by reference in its entirety).

Although it has been shown that sporozoites traverse human cells (Behet et al., Malar J 2014 Apr. 5; 13:136; Cha et al., J Exp Med 2015 Aug. 24; 212 (9): 1391-403; Dumoulin et al., PLOS One 2015 June 12; 10 (6): e0129623; van Schaijk et al., PLoS ONE, 3 (10). e3549 2008, each of which is incorporated herein by reference in its entirety), the molecular basis for the traversal process is largely unstudied. Antibodies against circumsporozoite protein (CSP) impair traversal (Dumoulin et al., 2015, which is incorporated herein by reference in its entirety), but this is likely due to inhibition of motility rather than a direct effect (Cha et al., J Exp Med 2016 Sep. 19; 213 (10): 2099-112, which is incorporated herein by reference in its entirety). Furthermore, antibodies induced by chloroquine prophylaxis with sporozoites interfere with cell traversal, and these may also target CSP (Behet et al., 2014, which is incorporated herein by reference in its entirety). Recently it was shown that glyceraldehyde 3-phosphate dehydrogenase (GAPDH) on the parasite surface interacts with CD68 on Kupffer cells during traversal (Cha et al., 2015, Cha et al., 2016, each of which is incorporated herein by reference in its entirety).

In rodent Plasmodium parasites such as P. berghei, two sporozoite microneme proteins have been identified that appear to be essential for cell traversal (sporozoite microneme protein essential for cell traversal [SPECT1; Ishino et al., PLOS Biol., 2 (2004), pp. 77-84, which is incorporated herein by reference in its entirety] and SPECT2 [Ishino et al., Cell. Microbiol., 7 (2005), pp. 199-208, which is incorporated herein by reference in its entirety], also called perforin-like protein 1 [PLP1][Kaiser et al., Mol. Biochem. Parasitol., 133 (2004), pp. 15-26, which is incorporated herein by reference in its entirety]. Even though genetic disruption of SPECT1 or SPECT2 rendered sporozoites unable to traverse murine cells, they still invaded hepatocytes in vitro (Ishino et al., 2004, Ishino et al., 2005, each of which is incorporated herein by reference in its entirety). When injected into rodents, sporozoites lacking SPECT1 or SPECT2 were impaired for liver infection, but a small number of sporozoites could still establish liver infection that resulted in subsequent patency. However, depletion of Kupffer cells allowed mutants to establish liver infection at levels comparable with wild-type parasites (Ishino et al., 2004, Ishino et al., 2005, each of which is incorporated herein by reference in its entirety). This data suggests that traversal by rodent-infecting sporozoites is important for navigating through the sinusoidal layer, but not for hepatocyte invasion, malarial exoerythrocytic forms development, or growth within erythrocytes (Ishino et al., 2004, Ishino et al., 2005, each of which is incorporated herein by reference in its entirety).

The ortholog of SPECT2 in P. yoelii, PLP1, has been shown to play a role in cell traversal. Although this protein is not required for hepatocyte entry, it plays a role in egress from transient vacuoles during traversal (Risco-Castillo et al., 2015, each of which is incorporated herein by reference in its entirety). Thus, sporozoites that infect rodents can traverse host cells by generating a vacuole at the entry step and use a perforin-like protein (e.g., SPECT2/PLP1) to escape from this compartment and/or a host cell, during cell exit.

Once sporozoites have invaded liver cells, they differentiate into merozoites, a replicative form of the parasite capable of lysing hepatocytes after multiple rounds of replication. Within a few days, a few hundred sporozoites can become hundreds of thousands of merozoites. When infected liver cells rupture, they release the merozoites into the bloodstream, where they invade red blood cells and begin the asexual reproductive stage, which is the symptomatic stage of the disease. Within a small number of days, millions of merozoites can be present in blood.

Malaria symptoms typically develop 4-8 days after initial red blood cell invasion. Replication cycle of merozoites within the red blood cells continues for 36-72 hours, until hemolysis, releasing the merozoites for another round of red blood cell infection. Thus, in synchronous infections (infections that originate from a single infectious bite), fever occurs every 36-72 hours, when infected red blood cells lyse and release endotoxins en masse.

Plasmodium spp. parasites gain entry into red blood cells through specific ligand-receptor interactions mediated by proteins on the surface of the parasite that interact with receptors on the host erythrocyte (mature red blood cell) or reticulocyte (immature red blood cell), whereas P. falciparum can invade and replicate in erythrocytes and reticulocytes, P. vivax and other species predominantly invade reticulocytes, which are less abundant than erythrocytes. Most of the erythrocyte-binding proteins or reticulocyte-binding proteins that have been associated with invasion are redundant or are expressed as a family of variant forms; however, for P. falciparum, two essential red blood cell receptors (basigin and complement decay-accelerating factor (also known as CD55)) have been identified.

Plasmodium vivax and Plasmodium ovale can also enter a dormant state in the liver, the hypnozoite.

Merozoites released from red blood cells can invade other red blood cells and continue to replicate, or in some cases, they differentiate into male or female gametocytes. Gametocytes concentrate in skin capillaries and are then taken up by the mosquito vector in another blood meal. In the gut of the mosquito, each male gametocyte produces eight microgametes after three rounds of mitosis; the female gametocyte matures into a macrogamete. Male microgametes are motile forms with flagellae and seek the female macrogamete. The male and female gametocytes fuse, forming a diploid zygote, which elongates into an ookinete; this motile form secretes a chitinase in order to enter the peritrophic membrane and traverse the midgut epithelium to the basal lateral side of the midgut, establishing itself in the basal lamina as an oocyst. Oocysts mature over 14-15 days, undergoing cycles of replication to form sporozoites that are ultimately liberated into the hemocoel, an environment rich in sugars and subtrates beneficial to the parasite's survival. Thousands of sporozoites can form from a single oocyst and become randomly distributed throughout the hemocoel. These sporozoites are motile and rapidly destroy the hemolymph, with only approximately 20% successfully invading the salivary gland. Following invasion of the salivary gland, sporozoites are re-programmed via an unknown mechanism to prepare for liver invasion. Evidence of this reprogramming has been demonstrated by the inability of midgut sporzoites (directly from oocysts) to invade hepatocytes, and also by the fact that sporzoites which have successfully invaded a salivary gland are unable to do re-invade another salivary gland if presented one. Salivary gland sporozoites alter mosquito behavior and salivary gland function, as less saliva is produced resulting in an increase in mosquito probing behavior, increasing the chances of transmission to a human host via a mosquito bite.

Some drugs that prevent Plasmodium spp. invasion or proliferation in the liver have prophylactic activity, drugs that block the red blood cell stage are required for the treatment of the symptomatic phase of the disease, and compounds that inhibit the formation of gametocytes or their development in the mosquito (including drugs that kill mosquitoes feeding on blood) are transmission-blocking agents (Phillips, et al. Malaria. Nat Rev Dis Primers 3, 17050 (2017), which is incorporated herein by reference in its entirety).

### B. Genome

Since completion of the first sequence of P. falciparum 3D7 genome in 2002, genomic research on Plasmodium parasites has rapidly advanced. Except for a short diploid phase after fertilization in the mosquito midgut, Plasmodium parasites are haploid throughout their life cycle. The genomes of different species range from 20 to 35 megabases, contain 14 chromosomes, a circular plastid genome of approximately 35 kilobases, and multiple copies of a 6 kilobase mitochondrial DNA. Comparison of genomes from different species showed that homologous genes are often found in synthetic blocks arranged in different orders among different chromosomes.

The adenine-thymine (AT) content of Plasmodium spp. can also be very different, e.g., ˜ 80% AT in P. falciparum, P. reichenowi, and P. gallinaceum, ˜ 75% AT in rodent Plasmodium parasites; and ˜ 60% AT in P. vivax, P. knowlesi, and P. cynomolgi. AT content is often higher in introns and intergenic noncoding regions than in protein-coding exons, with an average of 80.6% AT for the whole P. falciparum genome versus 86.5% for noncoding sequences. The high AT content of P. falciparum reflects large numbers of low-complexity regions, simple sequence repeats, and microsatellites, as well as a highly skewed codon usage bias. Polymorphisms of AT-rich repeats provide abundant markers for linkage mapping of drug resistance genes and for tracing the evolution and structure of parasite populations.

Plasmodium parasite genomes carry multigene families that serve important roles in parasite interactions with their hosts, including, for example, antigenic variation, signaling, protein trafficking, and adhesion. Among the gene families, genes encoding P. falciparum erythrocyte membrane protein 1 (PÆMP1) have been studied most extensively. Each individual P. falciparum parasite carries a unique set of 50 to 150 copies of the var gene in its genome, where switches of gene expression can produce antigenic variation. PfEMP1 plays an important role in the pathogenesis of clinical developments such as in cerebral and placental malaria, in which it mediates the cytoadherence of infected red blood cells (IRBCs; infected erythrocytes) in the deep tissues. Different PfEMP1 molecules bind to various host molecules, including α2-macroglobulin, CD36, chondroitin sulfate A (CSA), complement 1q, CR1, E-selectins and P-selectins, endothelial protein C receptor (EPCR), heparan sulfate, ICAM1, IgM, IgG, PECAM1, thrombospondin (TSP), and VCAM1. Such binding leads to activation of various host inflammatory responses. Hemoglobinopathies, including the hemoglobin C and hemoglobin S trait conditions, interfere with PfEMP1 display in knob structures of the iRBCs. This poor display of PfEMP1 on the host cell surface offers protection against malaria by reducing the cytoadherence and activation of inflammatory processes that promote the development of severe disease.

Members of the large Plasmodium interspersed repeat (pir) multigene family are named differently by parasite species, for example, yir in P. yoelii, bir in P. berghei, vir in P. vivax. Several P. falciparum gene families (stevor, rif, and P/MC-2TM) are classified with pir by their similar gene structures, which characteristically include a short first exon, a long second exon, and a third exon encoding a transmembrane domain. In a recent study, the pir genes from P. chabaudi (cir) were shown to be expressed in different cellular locations, within and on the surface of iRBCs, and in merozoites. Plasmodium parasites devote large portions of their genomes to gene families that ensure evasion of host immune defenses and protection of molecular processes essential to infection. These families emphasize the importance of research on their roles in parasite-host interactions and virulence, despite the difficulties inherent to their investigation.

An additional, exemplary polymorphic gene family comprises a group of 14 genes encoding proteins with six cysteines (6-Cys). These proteins often localize on the parasite surface interacting with host proteins and are expressed at different parasite developmental stages. 6-Cys proteins also demonstrate diverse functions and have been shown to play roles in, for example, parasite fertilization, mating interactions, evasion of immune responses, and invasion of hepatocytes. The proteins expressed in asexual stages are generally polymorphic and/or under selection, suggesting that they could be targets of the host immune response; however, their functions in parasite development remain largely unknown.

Plasmodium genomes can be highly polymorphic. Early studies demonstrated polymorphisms involving tens to hundreds of kilobases and that the chromosome structure in P. falciparum is largely conserved in central regions but extensively polymorphic is both length and sequence near the telomeres. Much of the subtelomeric variation was explained by recombination within blocks of repetitive sequences and families of genes.

The frequency of simple sequence repeats (microsatellites) in P. falciparum is estimated to be approximately one polymorphic microsatellite per kb DNA. Without wishing to be bound by any one theory, this high rate may reflect the AT-rich nature of the genome. Microsatellites seem to be less frequent in other Plasmodium species that have genomes with lower AT contents. In addition to the highly polymorphic and repetitive structure of Plasmodium genomes, there are also large numbers of Single Nucleotide Polymorphisms (SNPs) and Copy Number Variations (CNVs) (Su et al., Plasmodium Genomics and Genetics: New Insights into Malaria Pathogenesis, Drug Resistance, Epidemiology, and Evolution. Clin Microbiol Rev. 2019 Jul. 31; 32 (4), which is incorporated herein by reference in its entirety).

### C. Plasmodium Proteins

Plasmodium parasites are known to express various proteins at different stages of their lifecycles. Exemplary malarial proteins are described below, and SEQ ID NOs corresponding to exemplary amino acid sequences are provided in Table 2.

Circumsporozoite protein (CSP) is a multifunctional protein that is involved in Plasmodium life cycle, as it is required for the formation of sporozoites in the mosquito midgut, the release of sporozoites from the oocyst, invasion of salivary glands, attachment of sporozoites to hepatocytes in the liver, and sporozoite invasion of hepatocytes (see, e.g., Zhao et al. (2016) PLoS ONE 11 (8): e0161607, which is incorporated herein by reference in its entirety). CSP is present in all Plasmodium species, and although variation exists in the amino acid sequence across species, the overall domain structure of a central repeat region and nonrepeat flanking regions is well conserved (see, e.g., Zhao et al. (2016) PLoS ONE 11 (8): e0161607; Wahl et al. (2022) J. Exp. Med. 219: e20201313, each of which is incorporated herein by reference in its entirety). CSP sequences are known (see, e.g., UniProt accession numbers A0A2L1CF52, A0A2L,1CF88, C6FGZ3, C6FH2,7 C6FHG7, M1V060, M1V0A3, M1V0B0, M1V0C4, M1V0E0, M1V914, M1VFN9, M1VKZ2, P02893, Q5EIJ9, Q5EIK2, Q5EIK8, Q5EIL3, Q5EIL5, Q5EIL8, Q5R2L2, Q7K740, Q8I9G5, Q8I9J3, Q819J4), and Table 1 includes exemplary sequences for CSP P. falciparum isolates from Asia, South America and Africa.

TABLE 1
Exemplary Sequences for CSP P. falciparum isolates from Asia, South America and Africa

| Accession number | Country | References |
|---|---|---|
| AB121015.1 | 7G8 (Brazil) | Tanabe, K., et al., Genetic distance in housekeeping genes |
| | | between Plasmodium falciparum and Plasmodium reichenowi |
| | | and within P. falciparum. J. Mol. Evol. 59 (5), 687-694 (2004), |
| | | which is incorporated herein by reference. |
| AB121017.1 | Dd2 (Indochina) | Tanabe (2004) |
| AB121018.1 | HB3 (Honduras) | Tanabe (2004) |
| AB121020.1 | MAD20 (PNG) | Tanabe (2004) |
| AB121021.1 | RO-33 (Ghana) | Tanabe (2004) |
| AJ269946.1 | K1 (Thailand) | de Stricker, K., et al., Sequence variation in the non-repeat |
| | | region of the Plasmodium falciparum glutamate rich protein |
| | | (GLURP) from Brazil, Senegalese and Burmese field isolates |
| | | and from laboratory strains. (Unpublished), which is |
| | | incorporated herein by reference. |
| M15505.1 | Wellcome (West | Lockyer M J, Schwarz R T. Strain variation in the |
| | African) | circumsporozoite protein gene of Plasmodium falciparum. Mol |
| | | Biochem Parasitol. 2; 22(1): 101-8 (1987), which is |
| | | incorporated herein by reference. |
| M22982.1 | NF54 (Netherlands) | Caspers P, Gentz R, Matile H, Pink J R, Sinigaglia FThe |
| | | circumsporozoite protein gene from NF54, a Plasmodium |
| | | falciparum isolate used in malaria vaccine trials. Mol Biochem |
| | | Parasitol. 35(2): 185-9. (1989), which is incorporated herein by |
| | | reference. |
| AF540441.1, | India | Escalante A A, Grebert H M, Isea R, Goldman I F, Basco |
| AF540442.1, | | L, Magris M, Biswas S, Kariuki S and Lal A A. A study of genetic |
| AF540472.1, | | diversity in the gene encoding the circumsporozoite protein |
| AF540481.1 to | | (CSP) of Plasmodium falciparum from different transmission |
| AF540488.1 | | areas--XVI. Asembo Bay Cohort Project. Mol Biochem |
| | | Parasitol125(1-2): 83-90 (2002), which is incorporated herein |
| | | by reference. |
| AJ269943.1 | India | de Stricker, K. (Unpublished) |
| DQ521663.1 to | Iran | Zakeri S, et al., Restricted T-cell epitope diversity in the |
| DQ521752.1 | | circumsporozoite protein from Plasmodium falciparum |
| | | populations prevalent in Iran. .Am J Trop Med |
| | | Hyg.; 76(6): 1046-51 (2007), which is incorporated herein by |
| | | reference. |
| AJ269945.1, | Myanmar | de Stricker, K (Unpublished) |
| AJ269955.1- | | |
| AJ269960.1 | | |
| DQ193595.1 | Myanmar | Jalloh A, van Thien H, Ferreira M U, Ohashi J, Matsuoka |
| | | H, Kanbe T, Kikuchi A, Kawamoto F. Sequence variation in the |
| | | T-cell epitopes of the Plasmodium falciparum circumsporozoite |
| | | protein among field isolates is temporally stable: a 5-year |
| | | longitudinal study in southern Vietnam. J Clin Microbiol |
| | | 44(4): 1229-35 (2006), which is incorporated herein by |
| | | reference in its entirety. |
| FJ232142.1- | Thailand | Putaporntip C, Jongwutiwes S, Hughes A L. Natural selection |
| FJ232166.1, | | maintains a stable polymorphism at the circumsporozoite |
| FJ232168.1- | | protein locus of Plasmodium falciparum in a low endemic area. |
| FJ232364.1 | | Infect Genet Evol. 9(4): 567-73 (2009), which is incorporated |
| | | herein by reference in its entirety. |
| GQ890702.1- | Thailand | Jongwutiwes S, Putaporntip C, Hughes A L. Bottleneck effects |
| GQ890789.1 | | on vaccine-candidate antigen diversity of Plasmodium |
| | | parasites in Thailand. Vaccine. 19; 28(18): 3112-7. (2010), |
| | | which is incorporated herein by reference in its entirety. |
| M19752.1 | Thailand | del Portillo H A, Nussenzweig R S, Enea V. Circumsporozoite |
| | | gene of a Plasmodium falciparum strain from Thailand. Mol |
| | | Biochem Parasitol. 24(3): 289-94. (1987), which is |
| | | incorporated herein by reference in its entirety. |
| M83149.1, | Thailand | Jongwutiwes S, Tanabe K, Hughes M K, Kanbara H, Hughes A L. |
| M83150.1-M83174.1 | | Allelic variation in the circumsporozoite protein of Plasmodium |
| | | falciparum from Thai field isolates. Am J Trop Med Hyg; |
| | | 51(5): 659-68 (1994), which is incorporated herein by |
| | | reference in its entirety. |
| DQ193573.1- | Vietnam | Jalloh (2006) |
| DQ193594.1 | | |
| DQ193593.1- | Indonesia | Jalloh (2006) |
| DQ193594.1 | | |
| AB116602.1- | Vanuatu | Tanabe (2004) |
| AB116607.1 | | |
| AF540458.1- | Venezuela | Escalante (2002). |
| AF540460.1, | | |
| AF540464.1, | | |
| AF540466.1, | | |
| AF540469.1- | | |
| AF540471.1, | | |
| AF540478.1- | | |
| AF540479.1 | | |
| AJ269941.1, | Brazil | de Stricker (Unpublished) |
| AJ269971.1- | | |
| AJ269978.1 | | |
| K02194.1 | Brazil | Dame J B, Williams J L et al. Structure of the gene encoding the |
| | | immunodominant surface antigen on the sporozoite of the |
| | | human Plasmodium parasite Plasmodium falciparum. |
| | | Science. 10; 225(4662): 593-1984), which is incorporated herein |
| | | by reference in its entirety. |
| AF540443.1, | Asembo Bay | Escalante (2002). |
| AF540447.1- | | |
| AF540448.1, | | |
| AF540450.1- | | |
| AF540451.1, | | |
| AF540455.1- | | |
| AF540457.1, | | |
| AF540461.1- | | |
| AF540463.1, | | |
| AF540465.1, | | |
| AF540467.1, | | |
| AF540473.1 = | | |
| AF540477.1 | | |
| AF181833.1- | Gambia | Alloueche A, Silveira H, Conway D J, Bojang K, Doherty |
| AF181835.1, | | T, Cohen J, Pinder M, Greenwood B M. High-throughput |
| AY878598.1- | | sequence typing of T-cell epitope polymorphisms in |
| AY878641.1 | | Plasmodium falciparum circumsporozoite protein. Mol Biochem |
| | | Parasitol. 5; 106(2): 273-82 (2000), which is incorporated |
| | | herein by reference in its entirety. |
| AY878598.1 | Gambia | Weedall G D, Preston B M, Thomas A W, Sutherland C J, Conway |
| | | D J. Differential evidence of natural selection on two leading |
| | | sporozoite stage malaria vaccine candidate antigens. Int J |
| | | Parasitol. 37(1): 77-85 (2007), which is incorporated herein by |
| | | reference in its entirety. |
| AJ269948.1, | Senegal | de Stricker (Unpublished) |
| AJ269961.1- | | |
| AJ269970.1 | | |
| GQ119637.1- | Sierra Leone | Jalloh A, Jalloh M, Matsuoka H. T-cell epitope polymorphisms |
| GQ119678.1 | | of the Plasmodium falciparum circumsporozoite protein among |
| | | field isolates from Sierra Leone: age-dependent haplotype |
| | | distribution. Malar J. 5; 8: 120. (2009), which is incorporated |
| | | herein by reference in its entirety. |
| AF540444.1- | Cameroon | Escalante (2002). |
| AF540446.1, | | |
| AF540449.1, | | |
| AF540452.1- | | |
| AF540454.1, | | |
| AF540468.1, | | |
| AF540480.1 | | |

Exemplary CSP amino acid sequence is provided in SEQ ID NO: 1.

RH5 is found in Plasmodium falciparum (P. falciparum) and not found in the other species of Plasmodium that infect humans. RH5 orthologues are also found in other species belonging to the Lavarenia subgenus, which includes parasites that infect chimpanzees and gorillas, indicating a unique role in P. falciparum invasion of human erythrocytes. See, e.g., Ragotte, et al. Trends Parasitol. 36 (6) 2020, which is incorporated herein by reference in its entirety. RH5 is expressed during the mature schizont stages and can complex with Cysteine-rich Protective Antigen (CyRPA) and RH5-interacting Protein (Ripr) to form an elongated protein trimer on the merozoite surface that binds to erythrocyte surface protein basigin. See, e.g., Ragotte (2020), which is incorporated herein by reference in its entirety.

In humans, RH5 binding to basigin plays an essential role in invasion, acting downstream of membrane deformation. Binding of RH5 to basigin is required for the induction of a spike in calcium within the erythrocyte, which is blocked when merozoites attempt to invade in the presence of anti-RH5, anti-Ripr, or anti-basigin antibodies or soluble basigin. See, e.g., Ragotte (2020), which is incorporated herein by reference in its entirety.

RH5 is a 63 kDa protein expressed during the mature schizont stage. It is processed and cleaved to a 45 kDa form which is shed by the parasite. The structure of PfRH5 reveals a kite-like architecture formed from the coming together of two three-helical bundles. See, e.g., Ragotte (2020), which is incorporated herein by reference in its entirety.

RH5 sequences are known (see, e.g., UniProt accession numbers A0A159SK44, A0A159SK99, A0A159SKS8, A0A159SKW8, A0A159SL23, A0A159SL78, A0A159SL96, A0A159SLM7, A0A159SMC8, A0A159SMR9, A0A161FQT0, A0A1B1UZE2, A0A1B1UZE4, A0A1B1UZE5, A0A346RCI1, A0A346RCJ0, A0A346RCJ2, A0A346RCJ3, A0A346RCJ4, A0A346RCK4, A0A346RCK5, A0A346RCK6, A0A346RCK9, B2L3N7, Q8IFM5, each of which is incorporated herein by reference in its entirety), and exemplary RH5 amino acid sequence is provided in SEQ ID NO: 365.

P113 is a glycosylphosphatidylinositol (GPI)-linked protein that interacts directly with the N terminus of unprocessed RH5, providing a mechanism by which the RH5 invasion complex is tethered to the merozoite surface. See, e.g., Ragotte (2020). P113 orthologues are found in all Plasmodium species sequenced thus far, suggestive of a common and conserved function(s) (Bullen et al. (2022) Molecular Microbiology 117:1245-1262, which is incorporated herein by reference in its entirety). Despite this, in rodent model of malaria, P. berghei, p 113 knockout parasites were viable indicating the protein was not essential for asexual blood stage growth and invasion. The knockout parasites do, however, display defects in natural sporozoite transmission, leading to delayed patency in infected mice (Offeddu et al. (2014) Mol. Biochem. Parasitology 193:101-109, which is incorporated herein by reference in its entirety).

Plasmodium P113 sequences are known (see, e.g., Uniprot accession number Q8ILP3). Exemplary P113 amino acid sequence is provided in SEQ ID NO: 326.

Cysteine-Rich Protective Antigen (CyRPA) is a 43 kDa protein with a predicted N-terminal secretion signal. CyRPA is part of a multi-protein complex, including RH5 and Ripr, important for triggering Ca²⁺release and establishment of tight junctions. P/CyRPA is highly conserved, with only a single SNP above 5% prevalence, is essential for invasion (as conditional knockdown causes the loss of invasion activity), and has poor sero-reactivity from natural exposure (See, e.g., Ragotte (2020) which is incorporated herein by reference in its entirety).

Plasmodium CyRPA sequences are known (see, e.g., Uniprot accession number A0A2S1Q7P0, A0A2S1Q7P5, A0A2S1Q7Q4, Q8IFM8, each of which is incorporated herein by reference in its entirety). Exemplary CyRPA amino acid sequence is provided in SEQ ID NO: 329.

RH5-interacting Protein (Ripr) is an approximately 120 kDa protein and localized to micronemes during the schizont stage of the P. falciparum life cycle. The full-length 120 kDa protein is processed into two fragments of similar size, an N-terminal fragment (including EGF domains 1 and 2) and a C-terminal fragment (including EGF domains 3-10). Ripr colocalizes with RH5 and CyRPA during parasite invasion at the junction between merozoites and erythrocyte. Parasites with conditional knockouts of PÆRipr induce membrane deformation, but cannot complete invasion (See, e.g., Ragotte (2020) which is incorporated herein by reference in its entirety).

Plasmodium Ripr sequences are known (see, e.g., UniProt accession numbers A0A193PDI9, A0A193PDK3, A0A193PDK8, A0A193PDL3, A0A193PDL9, A0A193PDP4, A0A193PDQ8, A0A193PE01, A0A193PE05, A0A193PE07, 097302, A0A193PE17). Exemplary Ripr amino acid sequence is provided in SEQ ID NO: 332.

E140 is found in every Plasmodium species for which genomic sequence is available, and is well conserved, with amino acid identity ranging from 34-92% among species. See, e.g., Smith, et al. PLOS one 15.5 (2020): e0232234; http://doi: 10.1371/journal.pone.023223; and U.S. Patent Publication No. US 2019/0117752; each of which are incorporated herein by reference in their entirety. E140 is also highly conserved (95-99%) in P. falciparum strains isolated from different locations around the world, and exhibits a low mutation frequency. E140 is expressed at different life stages of Plasmodium parasites (specifically, E140 has been detected in sporozoites, liver, and blood stage parasites).

Protein structure algorithms predict that the E140 protein has five transmembrane domains, presumable spanning a parasite or host-derived membrane. E140 displays distinct patterns of protein expression in mature sporozoites, late liver, and late schizont stages. It traffics to the anterior and posterior ends of the sporozoite, the parasitophorous vacuole space of the late liver stage and around developing merozoites in the late schizont stage. It is also known to be expressed in mature salivary gland sporozoites as well as oocyst-derived sporozoites and oocysts.

E140 sequences are known (see, e.g., UniProt accession numbers A0A650D649, A0A650D653, A0A650D672, A0A650D687, A0A650D690, A0A650D694, A0A650D6A3, A0A650D6B8, A0A650D6L3, A0A650D6L7, Q81299, each of which is incorporated herein by reference in its entirety), and exemplary E140 amino acid sequence is provided in SEQ ID NO: 335.

CelTOS is required for sporozoite traversal through Kupfer cells during the liver invasion process. CelTOS forms a pore from within the cell, allowing for sporozoite egress into the liver. Antibody epitopes have been characterized from immunized mice and infected human populations (Pfand Pv). In mouse studies, immunization with CelTOS has been shown to provide protection and against challenge. Vaccination with CelTOS may generate antibodies that can bind the extracellular domain of the pore-forming complex, blocking complete formation of the pore and preventing sporozoite traversal into the liver. See, e.g., Jimah et al., Elife 2016 Dec. 1; 5: e20621. doi: 10.7554/eLife.20621, which is incorporated herein by reference in its entirety.

Plasmodium CelTOS sequences are known (see, e.g., Uniprot accession number M1ETJ8, Q53UB7, A0A2R4QLA5, A0A2R4QLI0, A0A2R4QLI5, A0A2R4QLJ1, A0A2R4QLJ4, M1ETJ8, Q53UB8, Q8I5P1, each of which is incorporated herein by reference in its entirety). Exemplary CelTOS amino acid sequence is provided in SEQ ID NO: 350.

SPECT1 and SPECT2 (the latter also sometimes referred to as perforin-like protein 1 (PLP1)) are essential Plasmodium proteins that may play a role in cell traversal. See Yang et al., Cell Rep. 2017 Mar. 28; 18 (13): 3105-3116. doi: 10.1016/j.celrep.2017.03.017, which is incorporated herein by reference in its entirety. Targeted disruption of P. falciparum SPECT1 or SPECT2 has been shown to reduce infectivity of sporozoites in liver-stage development in humanized mice. However, mechanisms of cell traversal of these two proteins are yet to be defined in P. falciparum. See Yang et al.

SPECT1 and SPECT2 are considered attractive pre-erythrocytic immune targets due to the key role they are thought to play in the crossing of the Plasmodium parasite across the dermis and the liver sinusoidal wall, prior to invasion of hepatocytes. Recombinant P. falciparum SPECT2 has been shown to cause lysis of red blood cells in a Ca²⁺-dependent manner, as has the MACPF/CDC domain of PISPECT2. PISPECT2 has also been implicated in the Ca²⁺-dependent egress of P. falciparum merozoites from red blood cells.

Plasmodium SPECT1 and SPECT2 sequences are known (see, e.g., UniProt accession numbers Q8IDR4 and Q9U₀J9, each of which is incorporated herein by reference in its entirety), and exemplary amino acid sequence is provided in SEQ ID NO: 353 and SEQ ID NO: 356, respectively.

Exported protein 1 (EXP1) is a single pass transmembrane protein with an N-terminal signal peptide expressed during intraerythrocytic stage and liver stage (see, e.g., Spielmann et al., Int J Med Microbiol. 2012 October; 302 (4-5): 179-86, which is incorporated herein by reference in its entirety). EXP1 was shown to initially localize to dense granules in merozoites and then be transported to parasitophorous vacuolar membrane (PVM) after invasion (see, e.g., Iriko et al., Parasitol Int. 2018 October; 67 (5): 637-639, which is incorporated herein by reference in its entirety). Once localized to the PVM, EXP1 forms homo-oligomers with a N-terminus that is exposed to the parasitophorous vacuolar lumen and a C-terminus that is exposed to the red blood cell cytosol (see, e.g., Mesén-Ramírez et al., PLOS Biol. 2019 Sep. 30; 17 (9): e3000473, which is incorporated herein by reference in its entirety).

EXP1 has been demonstrated to possess glutathione S-transferase (GST) activity that may protect Plasmodium from oxidative damage (see, e.g., Mesén-Ramírez et al., PLOS Biol 17 (9) 2019 Sep. 30; 17 (9): e3000473, which is incorporated herein by reference in its entirety). Recently, it was demonstrated that EXP1 is important for Plasmodium survival by maintaining correct localization of EXP2, a nutrient-permeable channel in the PVM (see, e.g., Mesén-Ramírez et al., PLOS Biol. 2019 Sep. 30; 17 (9): e3000473, which is incorporated herein by reference in its entirety).

P. falciparum EXP1 polypeptide sequences are known (see, e.g., UniProt accession number Q8IIF0, W7JTD3, Q25840, Q548U₂, Q5VKK2, Q5VKK5, Q5WRH8, Q6V9G₄, Q6V9G₆, Q6V9G₉, Q6V9H1, Q6V9H2, Q9U₅₉₀, P04923, P04926, each of which is incorporated herein by reference in its entirety). Exemplary EXP1 amino acid sequence is provided in SEQ ID NO: 314.

Upregulated in infective sporozoites gene 3 (UIS3) is a membrane-bound protein localized to sporozoite parasitophorous vacuolar membrane (PVM) in infected hepatocytes. UIS3 was shown to interact with liver fatty acid-binding protein (L-FABP) and be involved in fatty acid and/or lipid import during phases of Plasmodium growth (see, e.g., Sharma et al., J Biol Chem. 2008 Aug. 29; 283 (35): 24077-24088; Mikolajczak et al., Int J Parasitol. 2007 April; 37 (5): 483-9, each of which is incorporated herein by reference in its entirety).

After sporozoite invasion of host liver cells, there is synthesis of vital Plasmodium structural features (e.g., parasitophorous vacuolar membrane). During hepatocytic stages, the Plasmodium relies on host fatty acids for rapid synthesis of its membranes (see, e.g., Sharma et al., J Biol Chem. 2008 Aug. 29; 283 (35): 24077-24088, which is incorporated herein by reference in its entirety). UIS3 insertion in the PVM provides Plasmodium a method to import essential fatty acids and/or lipids during rapid sporozoites growth phases (see, e.g., Sharma et al., J Biol Chem. 2008 Aug. 29; 283 (35): 24077-24088, which is incorporated herein by reference in its entirety).

Immunization with UIS3-deficient Plasmodium berghei sporozoites protected against malaria in rodent malaria model (see, e.g., Mueller et al., Nature. 2005 Jan. 13; 433 (7022): 164-7, which is incorporated herein by reference in its entirety). UIS3-deficient Plasmodium berghei can start the transformation process in the liver; however, they show severe defects during transformation into trophozoites (see, e.g., Mueller et al., Nature. 2005 Jan. 13; 433 (7022): 164-7, which is incorporated herein by reference in its entirety). UIS3-deficient Plasmodium berghei are also unable to develop into mature liver schizonts and therefore abort malaria infection within the liver itself (see, e.g., Mueller et al., Nature. 2005 Jan. 13; 433 (7022): 164-7, which is incorporated herein by reference in its entirety). Further, it was previously demonstrated that UIS3 derived from Plasmodium berghei and UIS3 derived from Plasmodium falciparum exhibited a low (i.e. 34%) amino acid sequence identity (see, e.g., Mueller et al., Nature. 2005 Jan. 13; 433 (7022): 164-7, which is incorporated herein by reference in its entirety).

Plasmodium UIS3 sequences are known (see, e.g., UniProt accession number A0A509ARS3, A0A1C6YLP3, Q8IEU1, A0A384KLI1, A0A1G₄H423, A0A077YB01, Q9NFU4, each of which is incorporated herein by reference in its entirety). Exemplary UIS3 amino acid sequence is provided in SEQ ID NO: 359.

Upregulated in infective sporozoites gene 4 (UIS4) contains a single transmembrane domain and localizes to secretory organelles of sporozoites and to the parasitophorous vacuole membrane (PVM) of liver stages. UIS4 is not expressed in blood stages or early sporozoites that are produced in oocysts (see, e.g., Mackellar et al., Eukaryot Cell. 2010 May; 9 (5): 784-794, which is incorporated herein by reference in its entirety).

Deletion of UIS4 gene is associated with arrest of early liver stage development (see, e.g., Vaughan and Kappe, Cold Spring Harb Perspect Med. 2017 Jun. 1; 7 (6): a025486, which is incorporated herein by reference in its entirety). Recently, UIS4 was demonstrated to be involved in Plasmodium berghei survival by eluding host actin structures deployed as part of host cytosolic defense (see, e.g., Bana et al., iScience. 2022 Apr. 22; 25 (5): 104281. doi: 10.1016/j.isci.2022.104281. eCollection 2022 May 20, which is incorporated herein by reference in its entirety). P. falciparum has an ortholog to UIS4 named ETRAMP10.3 which is not able serve as a functional compliment to P. yoelii UIS4, indicating it likely serves a different function in P. falciparum's life cycle (see Mackellar et al., Eukaryot. Cell 9:784-94 (2010), which is incorporated herein by reference in its entirety).

Plasmodium falciparum early transcribed membrane protein 10.3 (ETRAMP10.3) is an approximately 10 kDa protein and member of the early transcribed membrane proteins multigene family, a family which is conserved across Plasmodium species and includes proteins located in the parasitophorous vacuole. Several ETRAMP proteins are specific to P. falciparum and not found in Plasmodium species that infect other organisms. ETRAMP10.3 is one example, which is expressed in both liver and blood stage P. falciparum parasites. ETRAMP10.3 transcription has been found to peak during the transition from ring to trophozoite stages of P. falciparum blood stage infection in a human host. ETRAMP10.3 localizes to the parasitophorous vacuole and is exported to a host erythrocyte during blood stage infection. Although ETRAMP10.3 is sometimes referred to as Upregulated in Infectious Sporozoites gene 4 (UIS4), ETRAMP10.3 is understood to be an ortholog of UIS4 on the basis of synteny and structural similarity. However, ETRAMP10.3 is not a functional ortholog of UIS4 and may play a different biological role. Although the biological function of ETRAMP10.3 has not yet been completely resolved, localization to vesicular structures in the host erythrocyte suggests a role in host-parasite interaction or in remodeling of infected erythrocyte. ETRAMP10.3 appears to play a key role in the Plasmodium life cycle. When ETRAMP10.3 is deleted, the deletion can lead to the disruption of liver-stage development in mice and asexual blood stage progression.

Although the terms “UIS4” and “ETRAMP10.3” in the literature are sometimes used to refer to different proteins, in context of the present disclosure, the terms “UIS4” and “ETRAMP10.3” interchangeably to refer to ETRAMP10.3.

Plasmodium ETRAMP10.3 sequences are known (see, e.g., UniProt accession number Q8UM9, which is incorporated herein by reference in its entirety). Exemplary ETRAMP10.3 amino acid sequence is provided in SEQ ID NO: 362.

Liver specific protein 1 (LISP-1) is expressed during Plasmodium development in hepatocytes and localized to the parasitophorous vacuolar membrane (PVM) (see, e.g., Ishino et al., Cell Microbiol. 2009 September; 11 (9): 1329-1339, which is incorporated herein by reference in its entirety). LISP-1 was shown to be expressed at high levels during late liver stages development and to be involved in PVM breakdown and subsequent merozoite release (see, e.g., Ishino et al., Cell Microbiol. 2009 September; 11 (9): 1329-1339, which is incorporated herein by reference in its entirety).

Intracellular Plasmodium deficient in LISP-1 develop into hepatic merozoites and display normal infectivity to erythrocytes (see, e.g., Ishino et al., Cell Microbiol. 2009 September; 11 (9): 1329-1339, which is incorporated herein by reference in its entirety). However, LISP1-deficient liver-stage Plasmodium do not rupture PVM and remain trapped inside hepatocytes (see, e.g., Ishino et al., Cell Microbiol. 2009 September; 11 (9): 1329-1339, which is incorporated herein by reference in its entirety).

Plasmodium LISP-1 sequences are known (see, e.g., UniProt accession number A0A2I0C2×6, Q8ILR5, each of which is incorporated herein by reference in its entirety). Exemplary LISP-1 amino acid sequence is provided in SEQ ID NO: 308.

Liver specific protein 2 (LISP-2) contains a modified 6-cys domain and is expressed during Plasmodium development in hepatocytes (see, e.g., Orito et al., Mol Microbiol. 2013 January; 87 (1): 66-79, which is incorporated herein by reference in its entirety). LISP-2 was shown to be expressed by liver stages Plasmodium, exported to hepatocytes, and be distributed throughout the host cell, including the nucleus (see, e.g., Orito et al., Mol Microbiol. 2013 January; 87 (1): 66-79, which is incorporated herein by reference in its entirety).

Intracellular Plasmodium deficient in LISP2 do not mature effectively during merozoites development (see, e.g., Orito et al., Mol Microbiol. 2013 January; 87 (1): 66-79, which is incorporated herein by reference in its entirety).

Plasmodium LISP-2 sequences are known (see, e.g., UniProt accession number A0A2I0BZR4, Q8I1×6, Q9UOD4, each of which is incorporated herein by reference in its entirety). Exemplary LISP-2 amino acid sequence is provided in SEQ ID NO: 311.

Thrombospondin-related adhesion protein (TRAP) contains an N-terminal domain that is commonly referred to as von Willebrand factor A domain, although it is most similar to an integrin I domain because it contains a metal ion-dependent adhesion site (MIDAS) with a bound Mg²⁺ ion that is required for sporozoite motility in vitro and infection in vivo (see, e.g., Lu et al., PLOS One. 2020; 15 (1): e0216260, which is incorporated herein by reference in its entirety). The I domain is inserted in an extensible β-ribbon and followed by a thrombospondin repeat (TSR) domain, a proline-rich segment at the C-terminus, a single-pass transmembrane domain, and a cytoplasmic domain (see, e.g., Lu et al., PLOS One. 2020; 15 (1): e0216260, which is incorporated herein by reference in its entirety). Sequence analysis of the proline-rich segment revealed the presence of SH3-domain binding PxxP motifs in Plasmodium TRAPs (Akhouri et al., Malar J. 2008 Apr. 22; 7:63. doi: 10.1186/1475-2875-7-63, which is incorporated herein by reference in its entirety).

TRAP is stored in the micronemes and becomes surface exposed at the sporozoite anterior tip when parasite comes in contact with host cells (Akhouri et al., Malar J. 2008 Apr. 22; 7:63. doi: 10.1186/1475-2875-7-63, which is incorporated herein by reference in its entirety). TRAP also plays an important role in liver cell invasion of sporozoites by helping sporozoites in gliding motility and in recognition of host receptors on the mosquito salivary gland and hepatocytes (Akhouri et al., Malar J. 2008 Apr. 22; 7:63. doi: 10.1186/1475-2875-7-63, which is incorporated herein by reference in its entirety).

Plasmodium TRAP sequences are known (see, e.g., UniProt accession numbers A0A5Q2EXK8, A0A5Q2EZD7, A0A5Q2F1F6, A0A5Q2F2B8, A0A5Q2F2H6, A0A5Q2F4G9, 076110, P16893, Q01507, Q26020, Q76NM2, W8VNB6, each of which is incorporated herein by reference in its entirety), and exemplary TRAP amino acid sequence is provided in SEQ ID NO: 287.

Liver-stage-associated protein (LSAP-1) has been shown to be found mainly at the periphery of the intracellular hepatic parasite throughout its development, but not in blood stage parasites and possibly in minor quantities in salivary gland sporozoites (see, e.g., Siau et al., PLOS Pathog. 2008 Aug. 8; 4 (8): e1000121, which is incorporated herein by reference in its entirety). LSAP-1 is among the most abundant transcripts in the salivary gland transcriptome but has not been detected in proteomic surveys of sporozoites. Rather, expression has only been detected only in liver stages (see, e.g., Siau et al., PLOS Pathog. 2008 Aug. 8; 4 (8): e1000121, which is incorporated herein by reference in its entirety).

Plasmodium LSAP-1 sequences are known (see, e.g., UniProt accession number Q81632, W7JR53, each of which is incorporated herein by reference in its entirety). Exemplary LSAP-1 amino acid sequence is provided in SEQ ID NO: 302.

Like LSAP-1, LSAP-2 is also among the most abundant transcripts in the salivary gland transcriptome but has not been detected in proteomic surveys of sporozoites. LSAP-2 has shown some efficacy as a vaccine when combined with other antigens. See, e.g., Halbroth et al., Infect Immun. 2020 Jan. 22; 88 (2): e00573-19. doi: 10.1128/IAI.00573-19. Print 2020 Jan. 22, which is incorporated herein by reference in its entirety.

Plasmodium LSAP-2 sequences are known (see, e.g., UniProt accession number Q8I632, W7JR53, each of which is incorporated herein by reference in its entirety). Exemplary LSAP-2 amino acid sequence is provided in SEQ ID NO: 305.

Liver-Stage Antigen 1 (LSA-1) is expressed after Plasmodium have invaded hepatocytes and antigen accumulates in the parasitophorous vacuole (see, e.g., Tucker, K. et al., 2016, ‘Pre-Erythrocytic Vaccine Candidates in Malaria’, in A. J. Rodriguez-Morales (ed.), Current Topics in Malaria, IntechOpen, London. 10.5772/65592, each of which is incorporated herein by reference in its entirety). The function of LSA-1 remains currently not known (see, e.g., Tucker, K. et al., 2016, ‘Pre-Erythrocytic Vaccine Candidates in Malaria’, in A. J. Rodriguez-Morales (ed.), Current Topics in Malaria, IntechOpen, London. 10.5772/65592, which is incorporated herein by reference in its entirety).

LSA-1 is a 230 kDa preerythrocytic stage protein containing a large central region consisting of over eighty 17 amino acid residue repeat units flanked by highly conserved C- and N-terminal regions (Richie, T. L. and Parekh, F. K. (2009) Malaria, which is incorporated herein by reference in its entirety). In Vaccines for Biodefense and Emerging and Neglected Diseases (Barrett, A. D. T. and Stanberry L. R., eds), pp. 1309-1364, Elsevier, which is incorporated herein by reference in its entirety). LSA1 is expressed only by liver stage Plasmodium and not by sporozoites (Richie, T. L. and Parekh, F. K. (2009) Malaria, which is incorporated herein by reference in its entirety). In Vaccines for Biodefense and Emerging and Neglected Diseases (Barrett, A. D. T. and Stanberry L. R., eds), pp. 1309-1364, Elsevier, which is incorporated herein by reference in its entirety). The repeat region results in significant variation of the protein between strains of Plasmodium falciparum (see, e.g., Tucker, K. et al., 2016, ‘Pre-Erythrocytic Vaccine Candidates in Malaria’, in A. J. Rodriguez-Morales (ed.), Current Topics in Malaria, IntechOpen, London. 10.5772/65592, which is incorporated herein by reference in its entirety).

Plasmodium LSA-1 sequences are known (see, e.g., UniProt accession number Q25886, Q25887, Q25893, Q26028, Q9GTX5, 096125, each of which is incorporated herein by reference in its entirety). Exemplary LSA-1 amino acid sequence is provided in SEQ ID NO: 290.

Liver stage antigen 3 (LSA-3) is a 200-kDa protein that is composed of three nonrepeating regions (NR-A, NR-B, and NR-C) flanking two short repeat regions and one long repeat region (see, e.g., Tucker, K. et al., 2016, ‘Pre-Erythrocytic Vaccine Candidates in Malaria’, in A. J. Rodriguez-Morales (ed.), which is incorporated herein by reference in its entirety), Current Topics in Malaria, IntechOpen, London. 10.5772/65592, which is incorporated herein by reference in its entirety). The nonrepeat regions are well conserved across geographically diverse strains of Plasmodium falciparum (see, e.g., Tucker, K. et al., 2016, ‘Pre-Erythrocytic Vaccine Candidates in Malaria’, in A. J. Rodriguez-Morales (ed.), Current Topics in Malaria, IntechOpen, London. 10.5772/65592, which is incorporated herein by reference in its entirety). The most significant variation is in the repeating regions due to organization and number of repeating subunits rather than composition of the repeating regions (see, e.g., Tucker, K. et al., 2016, ‘Pre-Erythrocytic Vaccine Candidates in Malaria’, in A. J. Rodriguez-Morales (ed.), Current Topics in Malaria, IntechOpen, London. 10.5772/65592, which is incorporated herein by reference in its entirety).

Recently, in vitro data has shown that antibodies against LSA-3 (in particular, the C-terminal portion of LSA-3) may provide some protection (see, e.g., Morita et al, Sci Rep. 2017 Apr. 5; 7:46086. doi: 10.1038/srep46086, which is incorporated herein by reference in its entirety).

Plasmodium LSA-3 sequences are known (see, e.g., UniProt accession number C7DU21, C7DU22, C7DU23, C7DU24, C7DU25, C7DU26, C7DU27, C7DU28, C7DU29, C7DU32, C7DU33, C7DU34, C7DU36, C7DU37, C7DU38, C7DU39, C7DU40, Q8I042, Q8I0A5, Q8I0DO, Q8IFR1, Q8IFR2, Q8IFR3, Q8IFR4, Q8IFR5, Q8IFR6, Q8IFR7, Q8IFR8, Q8IFR9, Q8IFS0, Q8IFS1, Q8IFS2, Q8IFS3, Q8IFS4, Q8IFS5, Q8IFS6, Q8IFS7, Q8IFS8, Q8IFS9, Q8IFT0, Q8IFT1, Q8IFT2, Q8IFT3, Q8IFT4, Q9UON9, Q9UOPO, A0A2I0BVD6, AOPFM9, 096275, each of which is incorporated herein by reference in its entirety). Exemplary LSA-3 amino acid sequence is provided in SEQ ID NO: 299.

Glutamic acid-rich protein (GARP) is a 80kDA protein which derives its name from its glutamic rich amino acid sequence which comprises 24% of all its residues. GARP is predominantly expressed in ring stages and trophozoites and has been shown to be a non-essential gene in cell culture but highly immunogenic in animal models. Although GARP is non-essential in cell culture, its localization to the periphery of infected erythrocytes may indicate a role in the sequestration of infected erythrocytes. GARP's involvement in sequestration has been proposed to occur by way of binding with an chloride/bicarbonate anion exchanger. Antibodies against GARP have been proposed to serve as signatures of protection against severe malaria and have shown efficacy in experimental trials in monkeys. (see, e.g., Hon et al, Trends in Paras 2020 August; 36 (8): 653-655. doi: 10.1016/j.pt.2020.05.012 and Lau et al, Plos Path. 2014 10, e1004135, each of which is incorporated herein by reference in its entirety). GARP sequences are known (see, e.g., UniProt accession number, Q9GTW3, Q9UON1, each of which is incorporated herein by reference in its entirety), and exemplary GARP amino acid sequence is provided in SEQ ID NO: 341.

Parasite-infected erythrocyte specific protein 2 (PIESP2) (see, e.g., UniProt accession number Q81488) is a highly immunogenic protein first expressed in the trophozoite stage and believed to be important for the clinical progression of cerebral malaria. Although this protein is predominantly found within erythrocytes, it has been shown to be present on the surface of erythrocytes, allowing them to adhere to endothelial cells in the vasculature of the brain. Antibodies against PIESP2 have been shown to prevent vascular adherence of Plasmodium and could prove valuable in preventing the preventing inflammatory response in the brain and impairment of the blood-brain barrier during cerebral malaria progression (see, e.g., Liu et al, Int J Biol Macromol. 2021 Apr. 30; 177:535-547. doi: 10.1016/j.ijbiomac.2021.02.145, which is incorporated herein by reference in its entirety). PIESP2 sequences are known (see, e.g., UniProt accession number Q8I488, which is incorporated herein by reference in its entirety), and exemplary PIESP2 amino acid sequence is provided in SEQ ID NO: 344.

Shizont egress antigen-1 (SEA1) is a large 244 kDA protein lacking transmembrane domains or known targeting signals. The function of SEA1 is not known; however, it has been shown to be effective in rodent vaccine studies and has even been proposed as a target of protective antibodies found in children. SEA1 received its name after it was reported that antibodies agasint this protein inhibited egress of Plasmodium merizoites. SEA1 localizes closely to centromers during nuclear division, implicating its role in the essential process of replication. To date, various studies have proposed a role for SEA1 in egress, but also in mitotic division of nuclei during replication. (Perrin et al. 2021, which is incorporated herein by reference in its entirety) (see, e.g., Perrin et al, mBio. 2021 Mar. 9; 12 (2): e03377-20. doi: 10.1128/mBio.03377-20, which is incorporated herein by reference in its entirety). SEA1 sequences are known (see, e.g., UniProt accession number A0A143ZXM2, which is incorporated herein by reference in its entirety), and exemplary SEA1 amino acid sequence is provided in SEQ ID NO: 347.

### D. Embodiments of Plasmodium Sequences

An exemplary wild-type CSP polypeptide amino sequence from Plasmoidum falciparum isolate 3D7 is presented in SEQ ID NO: 1, and includes the following: a secretory signal (amino acids 1-18); an N-terminal domain (amino acids 19-104); a junction region (amino acids 93-104), a central domain (amino acids 105-272); and a C-terminal domain (amino acids 273-397). In exemplary SEQ ID NO: 1, the N-terminal domain includes an N-terminal region (amino acids 19-80); an N-terminal end region (amino acids 81-92); and a junction region (amino acids 93-104). In exemplary SEQ ID NO: 1, the junction region includes an R1 region (amino acids 93-97) and a junction (SEQ ID NO: 132) at positions 98-104. In exemplary SEQ ID NO: 1, the central domain includes a minor repeat region (amino acids 105-128) and a major repeat region (amino acids 129-272). In exemplary SEQ ID NO: 1, the minor repeat region includes three repeats of the amino acid sequence NANPNVDP (SEQ ID NO: 102). In exemplary SEQ ID NO: 1, the major repeat region includes 35 repeats of the amino acid sequence NANP (SEQ ID NO: 147), wherein 35 repeats of the amino acid sequence NANP (SEQ ID NO: 147) are separated into two contiguous stretches, and wherein one stretch includes 17 repeats of the amino acid sequence NANP (SEQ ID NO: 147) and one includes 18 repeats of the amino acid sequence NANP (SEQ ID NO: 147) which flank an amino acid sequence of NVDP (SEQ ID NO: 144). The major repeat region includes the amino acid sequences NPNANP (SEQ ID NO: 150) and NANPNA (SEQ ID NO: 153). In exemplary SEQ ID NO: 1, the C-terminal domain includes a C-terminal region (amino acids 273-375), a serine-valine (amino acids 376-377), and a transmembrane domain (amino acids 378-397). In exemplary SEQ ID NO: 1, the C-terminal region includes a Th2R region (amino acids 314-327) and a Th3R region (amino acids 352-363).

TABLE 2
Exemplary amino acid sequences

| SEQ ID NO: | Protein |
|---|---|
| 1 | CSP |
| 287 | TRAP |
| 290 | LSA-1 |
| 293 | LSA-1a |
| 296 | LSA-1b |
| 299 | LSA-3 |
| 302 | LSAP1 (3D7) |
| 305 | LSAP2 (3D7) |
| 308 | LISP1 (3D7) |
| 311 | LISP2 (3D7) |
| 314 | EXP1 (3D7) |
| 317 | EXP2 (3D7) |
| 320 | SPELD (3D7) |
| 323 | PL (3D7) |
| 326 | P113 (3D7) |
| 329 | CyRPA (3D7) |
| 332 | RIPR (3D7) |
| 335 | E140 |
| 338 | AMA-1 |
| 341 | GARP |
| 344 | PIESP2 |
| 347 | SEA-1/SEP-1 |
| 350 | celTOS Pf (3D7) |
| 353 | SPECT1 (3D7) |
| 356 | SPECT2 |
| 359 | UIS3 |
| 362 | ETRAMP 10.3 |
| 365 | RH5 |
| 368 | IBIS1 |
| 371 | IBIS1 |
| 374 | IBIS1 |
| 377 | PY02667 |
| 380 | PY03652 |

### II. Plasmodium Polypeptide Constructs

The present disclosure, among other things, utilizes RNA technologies as a modality to express one or more Plasmodium polypeptide constructs (also referred to herein as “malaria polypeptide constructs” or “malarial polypeptide constructs” that include one or more malarial proteins, or one or more portions thereof, described herein. For example, in some embodiments, a Plasmodium polypeptide construct comprises one or more Plasmodium CSP polypeptide regions or portions thereof (e.g., immunogenic fragments of Plasmodium CSP). A portion of a CSP polypeptide or region can be a characteristic portion of CSP polypeptide or region. In some embodiments, a Plasmodium polypeptide construct additionally includes one or more additional amino acid sequences, such as a secretory signal (e.g., a heterologous secretory signal), a transmembrane region (e.g., a heterologous transmembrane region), a helper antigen, a multimerization region, and/or a linker, as described herein.

### A. CSP

In some embodiments, a Plasmodium polypeptide construct described herein includes one or more regions or portions of a CSP, e.g., Plasmodium CSP, e.g., P. falciparum CSP (SEQ ID NO:1), or a variant thereof (e.g., one or more immunogenic fragments of a CSP, e.g., Plasmodium CSP, e.g., P. falciparum CSP, or immunogenic variants thereof). A region of CSP (or CSP polypeptide region) may refer to an N-terminal region, an N-terminal end region, a junction region, a minor repeat region, a major repeat region or a C-terminal region. A portion of CSP (or CSP polypeptide portion) may refer to parts of a CSP polypeptide region or parts spanning two or more CSP polypeptide regions. In some embodiments, a CSP polypeptide portion comprises 25, 30, 35, 40, or 45 contiguous amino acids of the amino acid sequence according to SEQ ID NO:1. In some embodiments, a Plasmodium polypeptide construct does not include a secretory signal or a transmembrane region, e.g., corresponds to amino acids 19-375 of the amino acid sequence according to SEQ ID NO: 1 or corresponds to amino acids 19-376 or 19-377 of the amino acid sequence according to SEQ ID NO: 1, i.e., includes a serine or a serine and valine immediately after the C-terminal region.

In some embodiments, a Plasmodium polypeptide construct described herein includes a CSP minor repeat region. In some embodiments, a Plasmodium polypeptide construct described herein includes a portion of a CSP minor repeat region. In some embodiments, a portion of a CSP minor repeat region is about 10, 15, 20, 21, 22, or 23 contiguous amino acids in length. In some embodiments, a Plasmodium polypeptide construct described herein includes a CSP major repeat region. In some embodiments, a Plasmodium polypeptide construct described herein includes a portion of a CSP major repeat region. In some embodiments, a portion of a CSP major repeat region is about 100, 110, 120, 130, 135, 140, 141, or 142 amino acids in length. In some embodiments, a Plasmodium polypeptide construct described herein includes a CSP C-terminal region. In some embodiments, a Plasmodium polypeptide construct described herein includes a portion of a CSP C-terminal region. In some embodiments, a portion of a CSP C-terminal region is about 80, 90, 95, 100, 101, or 102 amino acids in length. In some embodiments, a Plasmodium polypeptide construct described herein includes a CSP N-terminal region. In some embodiments, a Plasmodium polypeptide construct described herein includes a portion of a CSP N-terminal region. In some embodiments, a portion of a CSP N-terminal region is about 45, 50, 55, 60, or 61 amino acids in length. In some embodiments, a Plasmodium polypeptide construct described herein includes a CSP N-terminal end region. In some embodiments, a Plasmodium polypeptide construct described herein includes a portion of a CSP N-terminal end region. In some embodiments, a portion of a CSP N-terminal end region is about 8, 9, 10, or 11 amino acids in length. In some embodiments, a Plasmodium polypeptide construct described herein includes a CSP junction region. In some embodiments, a Plasmodium polypeptide construct described herein includes a portion of a CSP junction region. In some embodiments, a portion of a CSP junction region is about 8, 9, 10, or 11 amino acids in length.

### Minor Repeat Region

In some embodiments, a Plasmodium polypeptide construct described herein includes one or more Plasmodium CSP minor repeat regions or portions thereof comprising one or more repeats of an amino acid sequence of NANPNVDP (SEQ ID NO: 102), and wherein the polypeptide does not comprise an amino acid sequence of NPNA, NPNANP (SEQ ID NO:150) or NANPNA (SEQ ID NO:153).

In some embodiments, a Plasmodium polypeptide construct described herein includes one or more Plasmodium CSP polypeptide regions or portions thereof comprising one or more (e.g., 2, 3, 4, 5, 6, 7, 8, 9, 10, 11 or 12) repeats of an amino acid sequence of NANPNVDP (SEQ ID NO: 102). In some embodiments, a Plasmodium polypeptide construct described herein includes one or more Plasmodium CSP polypeptide regions or portions thereof comprising two or more (e.g., between 2 and 12, or between 2 and 10, or between 2 and 9, or between 2 and 8, or between 4 and 12, or between 4 and 10) repeats of an amino acid sequence of NANPNVDP (SEQ ID NO: 102). In some embodiments, a Plasmodium polypeptide construct described herein includes one or more Plasmodium CSP polypeptide regions or portions thereof comprising exactly three repeats of an amino acid sequence of NANPNVDP (SEQ ID NO: 102). In some embodiments, a Plasmodium polypeptide construct described herein includes one or more Plasmodium CSP polypeptide regions or portions thereof comprising exactly eight repeats of an amino acid sequence of NANPNVDP (SEQ ID NO: 102). In some embodiments, a Plasmodium polypeptide construct described herein includes one or more Plasmodium CSP polypeptide regions or portions thereof comprising exactly nine repeats of an amino acid sequence of NANPNVDP (SEQ ID NO: 102).

In some embodiments, the repeats of the amino acid sequence of NANPNVDP (SEQ ID NO: 102) are all contiguous with each other. In some embodiments, the repeats of the amino acid sequence of NANPNVDP (SEQ ID NO: 102) are not all contiguous with each other. In some embodiments, a Plasmodium polypeptide construct described herein comprises four portions of a Plasmodium CSP minor repeat region, and wherein each portion of a Plasmodium CSP polypeptide comprises two contiguous repeats of the amino acid sequence of NANPNVDP (SEQ ID NO: 102).

### C-Terminal Region

In some embodiments, a Plasmodium polypeptide construct described herein includes one or more Plasmodium CSP C-terminal regions (e.g., amino acids 273-375 of SEQ ID NO:1), or one or more portions thereof, wherein the C-terminal region does not include a transmembrane region. In some embodiments, a Plasmodium polypeptide construct described herein includes exactly one Plasmodium CSP C-terminal region, and wherein the Plasmodium CSP C-terminal region comprises or consists of an amino acid sequence with at least 85% (e.g., at least 90%, 91%, 92%, 93%, 94%, 95%, 96%, 97%, 98%, or 99%) sequence identity to amino acids 273-375 of SEQ ID NO:1. In some embodiments, a Plasmodium polypeptide construct described herein includes two or more portions of a Plasmodium CSP C-terminal region (e.g., amino acids 273-375 of SEQ ID NO: 1).

In some embodiments, a Plasmodium polypeptide construct described herein includes one or more portions of the Plasmodium CSP C-terminal region, wherein each of the one or more portions comprises or consists of: (i) amino acids 314-327 of SEQ ID NO:1 (or amino acids 314-327 of SEQ ID NO:1 having 1, 2, 3, 4, or 5 amino acid substitutions); (ii) amino acids 352-363 of SEQ ID NO:1 (or amino acids 352-363 of SEQ ID NO:1 having 1, 2, 3, 4, or 5 amino acid substitutions); (iii) amino acids 326-374 of SEQ ID NO:1 (or amino acids 326-374 of SEQ ID NO:1 having 1, 2, 3, 4, or 5 amino acid substitutions), (iv) amino acids 364-377 of SEQ ID NO:1 (or amino acids 364-377 of SEQ ID NO:1 having 1, 2, 3, 4, or 5 amino acid substitutions), or (v) a combination thereof.

In some embodiments, a Plasmodium polypeptide construct described herein includes one portion of the Plasmodium CSP C-terminal region, wherein the portion comprises or consists of: (i) amino acids 314-327 of SEQ ID NO:1 (or amino acids 314-327 of SEQ ID NO:1 having 1, 2, 3, 4, or 5 amino acid substitutions); (ii) amino acids 352-363 of SEQ ID NO:1 (or amino acids 352-363 of SEQ ID NO:1 having 1, 2, 3, 4, or 5 amino acid substitutions); (iii) amino acids 326-374 of SEQ ID NO:1 (or amino acids 326-374 of SEQ ID NO:Z having 1, 2, 3, 4, or 5 amino acid substitutions), (iv) amino acids 364-377 of SEQ ID NO:1 (or amino acids 364-377 of SEQ ID NO: 1 having 1, 2, 3, 4, or 5 amino acid substitutions), or (v) a combination thereof.

In some embodiments, a Plasmodium polypeptide construct described herein includes one or more portions of the Plasmodium CSP C-terminal region, wherein the one or more portions collectively comprise or consist of: (i) amino acids 314-327 of SEQ ID NO:1 (or amino acids 314-327 of SEQ ID NO:1 having 1, 2, 3, 4, or 5 amino acid substitutions); (ii) amino acids 352-363 of SEQ ID NO:1 (or amino acids 352-363 of SEQ ID NO:1 having 1, 2, 3, 4, or 5 amino acid substitutions); (iii) amino acids 326-374 of SEQ ID NO:1 (or amino acids 326-374 of SEQ ID NO:1 having 1, 2, 3, 4, or 5 amino acid substitutions), (iv) amino acids 364-377 of SEQ ID NO:1 (or amino acids 364-377 of SEQ ID NO:1 having 1, 2, 3, 4, or 5 amino acid substitutions), or (v) a combination thereof.

In some embodiments, a Plasmodium polypeptide construct described herein comprises a serine amino acid residue immediately following a Plasmodium CSP C-terminal region described herein. In some embodiments, a Plasmodium polypeptide construct described herein comprises a serine-valine amino acid sequence immediately following a Plasmodium CSP C-terminal region described herein.

### Junction Region

In some embodiments, a Plasmodium polypeptide construct described herein includes one or more Plasmodium CSP junction regions or portions thereof. In some embodiments, a Plasmodium polypeptide construct described herein includes two or more Plasmodium CSP junction regions or portions thereof. In some embodiments, a Plasmodium polypeptide construct described herein includes exactly one Plasmodium CSP junction region. In some embodiments, a Plasmodium CSP junction region comprises or consists of amino acids 93-104 of SEQ ID NO:1 (or amino acids 93-104 of SEQ ID NO:1 having 1, 2, 3, 4, or 5 amino acid substitutions). In some embodiments, a Plasmodium polypeptide construct described herein includes one or more portions of a Plasmodium CSP junction region. In some embodiments, one or more portions of a Plasmodium CSP junction region comprise a deletion of one or more of K93, L94, K95, Q96 and P97, wherein the amino acid numbering is relative to SEQ ID NO:1. In some embodiments, one or more portions of a Plasmodium CSP junction region comprise a deletion of K93, L94, K95, and Q96, wherein the amino acid numbering is relative to SEQ ID NO: 1. In some embodiments, one or more portions of a Plasmodium CSP junction region comprise a deletion of K93, L94, K95, Q96 and P97, wherein the amino acid numbering is relative to SEQ ID NO: 1.

In some embodiments, a Plasmodium polypeptide construct described herein includes one or more Plasmodium CSP junction region variants. In some embodiments, a Plasmodium CSP junction region variant comprises one or more amino acid substitution mutations. In some embodiments, one or more substitution mutations comprise a K93A mutation, an L94A mutation, or both, wherein the amino acid numbering is relative to SEQ ID NO: 1. In some embodiments, a Plasmodium CSP junction region variant comprises the amino acid sequence of AAKQ (SEQ ID NO: 426).

### N-Terminal End Region

In some embodiments, a Plasmodium polypeptide construct described herein includes one or more Plasmodium CSP N-terminal end regions or portions thereof. In some embodiments, a Plasmodium polypeptide construct described herein includestwo or more Plasmodium CSP N-terminal end regions or portions thereof. In some embodiments, a Plasmodium CSP N-terminal end region comprises or consists of amino acids 81-92 of SEQ ID NO: 1 (or amino acids 81-92 of SEQ ID NO:1 having 1, 2, 3, 4, or 5 amino acid substitutions). In some embodiments, a Plasmodium polypeptide construct described herein does not comprise a Plasmodium CSP N-terminal end region or any portion thereof (i.e., lacks or excludes a Plasmodium CSP N-terminal end region or any portion thereof).

### N-Terminal Region

In some embodiments, a Plasmodium polypeptide construct described herein includes one or more Plasmodium CSP N-terminal regions or portions thereof. In some embodiments, a Plasmodium polypeptide construct described herein includes two or more Plasmodium CSP N-terminal regions or portions thereof. In some embodiments, a Plasmodium CSP N-terminal region comprises or consists of amino acids 19-80 of SEQ ID NO:1. In some embodiments, a Plasmodium CSP N-terminal region comprises or consists of an amino acid sequence with at least 85% (e.g., at least 90%, 91%, 92%, 93%, 94%, 95%, 96%, 97%, 98%, or 99%) sequence identity to amino acids 19-80 of SEQ ID NO:1. In some embodiments, a Plasmodium polypeptide construct described herein does not comprise a Plasmodium CSP N-terminal region or any portion thereof (i.e., lacks or excludes a Plasmodium CSP N-terminal region or any portion thereof).

### Major Repeat Region

In some embodiments, a Plasmodium polypeptide construct described herein includes one or more Plasmodium CSP major repeat regions or portions thereof. In some embodiments, a Plasmodium polypeptide construct described herein includes exactly one Plasmodium CSP major repeat region or portion thereof, and the Plasmodium CSP major repeat region or portion thereof comprises a total of at least 2 and at most 35 repeats of the amino acid sequence NANP (SEQ ID NO: 147). In some embodiments, a Plasmodium CSP major repeat region or portion thereof comprises two contiguous stretches of repeats of the amino acid sequence NANP (SEQ ID NO: 147), and wherein the two contiguous stretches of the repeats of the amino acid sequence NANP (SEQ ID NO: 147) flank an amino acid sequence of NVDP (SEQ ID NO: 144). In some embodiments, a Plasmodium CSP major repeat region comprises, in N-terminus to C-terminus order, 17 repeats of the amino acid sequence NANP (SEQ ID NO: 147), an amino acid sequence of NVDP (SEQ ID NO: 144), and 18 repeats of the amino acid sequence NANP (SEQ ID NO: 147). In some embodiments, a portion of the Plasmodium CSP major repeat region consists of at most 18 contiguous repeats of the amino acid sequence NANP (SEQ ID NO: 147). In some embodiments, a portion of the Plasmodium CSP major repeat region consists of 2 contiguous repeats of the amino acid sequence NANP (SEQ ID NO: 147). The one or more Plasmodium CSP major repeat region or portion thereof always contains at least one repeat of the amino acid sequence of NPNANP (SEQ ID NO: 150) or NANPNA (SEQ ID NO: 153). In some embodiments, a Plasmodium CSP major repeat region comprises or consists of an amino acid sequence with at least 85% (e.g., at least 90%, 91%, 92%, 93%, 94%, 95%, 96%, 97%, 98%, or 99%) sequence identity to amino acids 129-272 of SEQ ID NO:1. In some embodiments, a Plasmodium polypeptide construct described herein does not comprise a Plasmodium CSP major repeat region or a portion of a Plasmodium CSP major repeat region comprising the amino acid sequence NPNA (SEQ ID NO: 141) (i.e., lacks or excludes a Plasmodium CSP major repeat region or a portion of a Plasmodium CSP major repeat region comprising the amino acid sequence NPNA [SEQ ID NO: 141]).

In some embodiments, a Plasmodium polypeptide construct described herein optionally includes one or more of the following Plasmodium CSP polypeptide regions or portions thereof, and if present, are in the following N-terminus to C-terminus order: (i) one or more Plasmodium CSP N-terminal regions or portions thereof, (ii) one or more Plasmodium CSP N-terminal end regions or portions thereof, (iii) one or more Plasmodium CSP junction regions, portions thereof, or variants thereof, (iv) one or more repeats of the amino acid sequence of NANPNVDP (SEQ ID NO: 102), (v) one or more Plasmodium CSP major repeat regions or portions thereof, and (vi) one or more Plasmodium CSP C-terminal regions or portions thereof.

In some embodiments, a Plasmodium polypeptide construct described herein optionally includes one or more of the following Plasmodium CSP polypeptide regions or portions thereof, and if present, are in the following N-terminus to C-terminus order: (i) one Plasmodium CSP N-terminal region or portion thereof, (ii) one Plasmodium CSP N-terminal end region or portion thereof, (iii) one Plasmodium CSP junction region, portion thereof, or variant thereof, (iv) one or more Plasmodium CSP minor repeat sequences, (v) one Plasmodium CSP major repeat region or portion thereof, and (vi) one Plasmodium CSP C-terminal region or portion thereof.

### B. Secretory Signals

In some embodiments, a Plasmodium polypeptide construct described herein includes a secretory signal, e.g., that is functional in mammalian cells. In some embodiments, a secretory signal comprises or consists of a Plasmodium secretory signal. In some embodiments, a Plasmodium secretory signal comprises or consists of a Plasmodium CSP secretory signal. In some embodiments, a Plasmodium CSP secretory signal is from Plasmodium falciparum. In some embodiments, a Plasmodium CSP secretory signal is from Plasmodium falciparum isolate 3D7 (SEQ ID NO. 174).

In some embodiments, a utilized secretory signal is a heterologous secretory signal. In some embodiments, a heterologous secretory signal comprises or consists of a non-human secretory signal. In some embodiments, a heterologous secretory signal comprises or consists of a viral secretory signal. In some embodiments, a viral secretory signal comprises or consists of an HSV secretory signal (e.g., an HSV-1 or HSV-2 secretory signal). In some embodiments, an HSV secretory signal comprises or consists of an HSV glycoprotein D (gD) secretory signal. In some embodiments, a secretory signal comprises or consists of an Ebola virus secretory signal. In some embodiments, an Ebola virus secretory signal comprises or consists of an Ebola virus spike glycoprotein (SGP) secretory signal.

The present disclosure provides the insight that in some embodiments, inclusion of a viral secretory signal in a polypeptide construct encoding a parasitic antigen can have one or more improved characteristics. In some embodiments, a polypeptide construct comprises a viral secretory signal and one or more parasitic antigens. In some embodiments, one or more parasitic antigens comprise one or more malarial antigens. In some embodiments, one or more malarial antigens comprise one or more Plasmodium CSP polypeptide regions or portions thereof as described herein. In some embodiments, a viral secretory signal comprises an HSV secretory signal (e.g., an HSV-1 or HSV-2 secretory signal. In some embodiments, an HSV secretory signal comprises an HSV glycoprotein D (gD) secretory signal. In some embodiments, an HSV gD secretory signal comprises an HSV-1 gD secretory signal. In some embodiments, an HSV gD secretory signal comprises an HSV-2 gD secretory signal.

In some embodiments, a polypeptide construct comprises an HSV-1 gD secretory signal and one or more parasitic antigens. In some embodiments, a polypeptide construct comprises an HSV-1 gD secretory signal and one or more malarial antigens. In some embodiments, a polypeptide construct comprises an HSV-1 gD secretory signal and one or more Plasmodium CSP polypeptide regions or portions thereof as described herein.

In some embodiments, a polypeptide construct comprises an HSV-2 gD secretory signal and one or more parasitic antigens. In some embodiments, a polypeptide construct comprises an HSV-2 gD secretory signal and one or more malarial antigens. In some embodiments, a polypeptide construct comprises an HSV-2 gD secretory signal and one or more Plasmodium CSP polypeptide regions or portions thereof as described herein.

In some embodiments, a polypeptide construct comprising a parasitic antigen and a viral secretory signal has one or more improved characteristics. In some embodiments, an improved characteristic is, e.g., increased expression (e.g., increased ex vivo expression (e.g., extracellular expression), or increased in vivo expression (e.g., extracellular expression)), improved inhibition of sporozoite traversal, and/or improved sporozoite binding. In some embodiments, a Plasmodium polypeptide construct comprises a viral secretory signal and has one or more improved characteristics. In some embodiments, a Plasmodium polypeptide construct comprises an HSV secretory signal (e.g., an HSV-1 or HSV-2 secretory signal) and has one or more improved characteristics. In some embodiments, a Plasmodium polypeptide construct comprises an HSV glycoprotein D (gD) secretory signal and has one or more improved characteristics. In some embodiments, a Plasmodium polypeptide construct comprises an HSV-2 glycoprotein D (gD) secretory signal and has one or more improved characteristics. In some embodiments, a Plasmodium polypeptide construct comprises an HSV-1 glycoprotein D (gD) secretory signal and has one or more improved characteristics.

In some embodiments, a Plasmodium polypeptide construct comprising a viral secretory region has increased expression (e.g., increased ex vivo expression (e.g., extracellular expression), or increased in vivo expression (e.g., extracellular expression). For example, in some embodiments, a Plasmodium polypeptide construct comprising a heterologous secretory region has increased ex vivo expression, e.g., in mammalian cells. In some embodiments, mammalian cells can be in a (e.g., HEK293T cells) as described in Example 1 below.

In some embodiments, a Plasmodium polypeptide construct comprises a viral secretory signal and has increased expression in mammalian cells (e.g., HEK293T cells) relative to an otherwise identical construct with a non-viral secretory signal (e.g., a Pf secretion signal). In some embodiments, a Plasmodium polypeptide construct comprises an HSV secretory signal (e.g., an HSV-1 or HSV-2 secretory signal) and has increased expression in mammalian cells (e.g., HEK293T cells) relative to an otherwise identical construct with a non-viral secretory signal (e.g., a Pf secretion signal). In some embodiments, a Plasmodium polypeptide construct comprises an HSV glycoprotein D (gD) secretory signal and has increased expression in mammalian cells (e.g., HEK293T cells) relative to an otherwise identical construct with a non-viral secretory signal (e.g., a Pf secretion signal).

In some embodiments, a Plasmodium polypeptide construct comprising a heterologous secretory region has improved inhibition of sporozoite traversal. For example, in some embodiments, a Plasmodium polypeptide construct comprising a heterologous secretory region has improved production of antibodies that inhibit sporozoite traversal, e.g., as measured using a traversal assay, e.g., as described in Example 2 below.

In some embodiments, a Plasmodium polypeptide construct comprises a viral secretory signal and has improved inhibition of sporozoite traversal relative to an otherwise identical construct with a non-viral secretory signal (e.g., a Pf secretion signal). In some embodiments, a Plasmodium polypeptide construct comprises an HSV secretory signal (e.g., an HSV-1 or HSV-2 secretory signal) and has improved inhibition of sporozoite traversal relative to an otherwise identical construct with a non-viral secretory signal (e.g., a Pf secretion signal). In some embodiments, a Plasmodium polypeptide construct comprises an HSV glycoprotein D (gD) secretory signal and has improved inhibition of sporozoite traversal relative to an otherwise identical construct with a non-viral secretory signal (e.g., a Pf secretion signal).

In some embodiments, a Plasmodium polypeptide construct comprising a heterologous secretory region has improved sporozoite binding. For example, in some embodiments, a Plasmodium polypeptide construct comprising a heterologous secretory region has improved binding to native PfCSP on PfCSP-expressing Plasmodium berghei (PbPf) sporozoites, e.g., as described in Example 2 below.

In some embodiments, a Plasmodium polypeptide construct comprises a viral secretory signal and has improved sporozoite binding relative to an otherwise identical construct with a non-viral secretory signal (e.g., a Pf secretion signal). In some embodiments, a Plasmodium polypeptide construct comprises an HSV secretory signal (e.g., an HSV-1 or HSV-2 secretory signal) and has improved sporozoite binding relative to an otherwise identical construct with a non-viral secretory signal (e.g., a Pf secretion signal). In some embodiments, a Plasmodium polypeptide construct comprises an HSV glycoprotein D (gD) secretory signal and has improved sporozoite binding relative to an otherwise identical construct with a non-viral secretory signal (e.g., a Pf secretion signal).

In some embodiments, a secretory signal is characterized by a length of about 15 to 30 amino acids.

In many embodiments, a secretory signal is positioned at the N-terminus of a Plasmodium polypeptide construct described herein. In some embodiments, a secretory signal preferably allows transport of a Plasmodium polypeptide construct with which it is associated into a defined cellular compartment, preferably a cell surface, endoplasmic reticulum (ER) or endosomal-lysosomal compartment.

In some embodiments, a secretory signal is selected from an S1S2 secretory signal (aa 1-19), an immunoglobulin secretory signal (aa 1-22), a human SPARC secretory signal, a human insulin isoform 1 secretory signal, a human albumin secretory signal, etc. Those skilled in the art will be aware of other secretory signal such as, for example, as disclosed in WO2017/081082, which is incorporated herein by reference in its entirety (e.g., SEQ ID NOS: 1-1115 and 1728, or fragments variants thereof). In some embodiments, a Plasmodium polypeptide construct described herein does not comprise a secretory signal.

In some embodiments, a secretory signal is one comprising an amino acid sequence according to a SEQ ID NO listed in Table 3, or a secretory signal having 1, 2, 3, 4, or 5 amino acid differences relative thereto. In some embodiments, a signal sequence is selected from those provided in Table 3 below and/or those encoded by the sequences provided in Table 4 below.

TABLE 3
Exemplary secretory signals

| SEQ ID | | SEQ ID | |
|---|---|---|---|
| NO: | Signal | NO: | Signal |
| 159 | HSV-1 gD SP | 198 | TRIO |
| 168 | HSV-2 gD SP | 201 | human Ig heavy chain signal |
| | | | peptide 1 |
| 171 | HSV-2 | 204 | human Ig heavy chain signal |
| | | | peptide 2 |
| 174 | Csp (isolate 3D7) | 207 | human Ig heavy chain signal |
| | | | peptide 3 |
| 165 | HSV-1 gD SP 3 | 210 | human Ig heavy chain signal |
| | | | peptide 4 |
| 177 | Ebola spike glycoprotein GP | 213 | human Ig heavy chain signal |
| | | | peptide 5 |
| 180 | SARS-CoV-2-S | 216 | human Ig heavy chain signal |
| | | | peptide 6 |
| 183 | human Ig heavy chain signal | 219 | human Ig heavy chain signal |
| | peptide (huSec) | | peptide 7 |
| 186 | HuIgGk signal peptide | 222 | human Ig heavy chain signal |
| | | | peptide 8 |
| 189 | IgE heavy chain epsilon-1signal | 225 | human Ig kappa chain signal |
| | peptide | | peptide 1 |
| 192 | Japanese encephalitis PRM signal | 228 | human Ig kappa chain signal |
| | sequence | | peptide 2 |
| 195 | VSVg protein signal sequence | | |

TABLE 4
Exemplary polynucleotide sequences encoding secretory signals

| SEQ ID NO: | Signal |
|---|---|
| 158 | HSV-1 gD SP wild-type |
| 161 | HSV-1 gD SP |
| | optimized nt sequence |
| 179 | SARS-CoV-2-S |
| 182 | human Ig heavy chain signal peptide (huSec) |
| 200 | human Ig heavy chain signal peptide 1 |
| 203 | human Ig heavy chain signal peptide 2 |
| 206 | human Ig heavy chain signal peptide 3 |
| 209 | human Ig heavy chain signal peptide 4 |
| 212 | human Ig heavy chain signal peptide 5 |
| 215 | human Ig heavy chain signal peptide 6 |
| 218 | human Ig heavy chain signal peptide 7 |
| 221 | human Ig heavy chain signal peptide 8 |
| 224 | human Ig kappa chain signal peptide 1 |
| 227 | human Ig kappa chain signal peptide 2 |

c. Transmembrane Regions

In some embodiments, a Plasmodium polypeptide construct described herein includes a transmembrane region. In some embodiments, a transmembrane region comprises or consists of a Plasmodium transmembrane region. In some embodiments, a utilized transmembrane region is one that is normally associated with CSP in nature. In some embodiments, a Plasmodium transmembrane region comprises or consists of a Plasmodium CSP glycosylphosphatidylinositol (GPI) anchor region. In some embodiments, a Plasmodium CSP GPI anchor region is from Plasmodium falciparum. In some embodiments, a Plasmodium CSP GPI anchor region is from Plasmodium falciparum isolate 3D7 (SEQ ID NO.231), e.g., amino acids 378-397 of SEQ ID NO:1. In some embodiments, a utilized transmembrane region is a heterologous transmembrane region.

In some embodiments, a transmembrane region is located at the N-terminus of a Plasmodium polypeptide construct. In some embodiments, a transmembrane region is located at the C-terminus of a Plasmodium polypeptide construct. In some embodiments, a transmembrane region is not located at the N-terminus or C-terminus of a Plasmodium polypeptide construct.

Transmembrane regions are known in the art, any of which can be utilized in a Plasmodium polypeptide construct described herein. In some embodiments, a transmembrane region comprises or is a transmembrane domain of Hemagglutinin (HA) of Influenza virus, Env of HIV-1, equine infectious anaemia virus (EIAV), murine leukaemia virus (MLV), mouse mammary tumor virus, G protein of vesicular stomatitis virus (VSV), Rabies virus, or a seven transmembrane domain receptor.

In some embodiments, a heterologous transmembrane region does not comprise a hemagglutin transmembrane region. In some embodiments, a heterologous transmembrane region comprises or consists of a non-human transmembrane region. In some embodiments, a heterologous transmembrane region comprises or consists of a viral transmembrane region. In some embodiments, a heterologous transmembrane region comprises or consists of an HSV transmembrane region, e.g., an HSV-1 or HSV-2 transmembrane region. In some embodiments, an HSV transmembrane region comprises or consists of an HSV gD transmembrane region, e.g., comprising or consisting of an amino acid sequence according to SEQ ID NO:234.

In some embodiments, a heterologous transmembrane region comprises or consists of a human transmembrane region. In some embodiments, a human transmembrane region comprises or consists of a human decay accelerating factor glycosylphosphatidylinositol (hDAF-GPI) anchor region. In some embodiments, an hDAF-GPI anchor region comprises or consists of an amino acid sequence according to SEQ ID NO:237.

In some embodiments, a Plasmodium polypeptide construct described herein does not comprise a transmembrane region.

### D. Helper Antigens

In some embodiments, a Plasmodium polypeptide construct described herein includes one or more helper antigens. Those skilled in the art are aware of a variety of potentially useful helper antigens, including those described in, e.g., WO2020128031 (which is incorporated herein by reference in its entirety) (e.g., P2 tetanus toxin, PADRE peptide, Hepatitis B surface antigen (HBsAg)). In some embodiments, a helper antigen is a malarial protein (e.g., a malarial protein described herein), provided that the antigen is not a CSP polypeptide or portion thereof. In some embodiments, a helper antigen is Plasmodium 2-phospho-D-glycerate hydro-lyase antigen, Plasmodium liver stage antigen 1 (a), (LSA-1 (a)), Plasmodium liver stage antigen 1 (b) (LSA-1 (b)), Plasmodium thrombospondin-related anonymous protein (TRAP), Plasmodium liver stage associated protein 1 (LSAP1), Plasmodium liver stage associated protein 2 (LSAP2), Plasmodium UIS3, Plasmodium UIS4, Plasmodium ETRAMP10.3, Plasmodium liver specific protein 1 (LISP-1), Plasmodium liver specific protein 2 (LISP-2), Plasmodium liver stage antigen 3 (LSA-3), Plasmodium EXP1, Plasmodium E140, Plasmodium reticulocyte-binding protein homolog 5 (Rh5), Plasmodium glutamic acid-rich protein (GARP), Plasmodium parasite-infected erythrocyte surface protein 2 (PIESP2), Plasmodium Cysteine-Rich Protective Antigen (CyRPA), Plasmodium Ripr, Plasmodium P113, or a combination thereof.

In some embodiments, a helper antigen comprises or consists of a P. falciparum 2-phospho-D-glycerate hydro-lyase antigen, e.g., comprising or consisting of an amino acid sequence according to SEQ ID NO: 240. In some embodiments, a helper antigen comprises or consists of a P. falciparum liver-stage antigen 3, e.g., comprising or consisting of an amino acid sequence according to SEQ ID NO:243. In some embodiments, a helper antigen comprises or consists of an Anopheles antigen, e.g., an Anopheles gambiae TRIO, e.g., comprising or consisting of an amino acid sequence according to SEQ ID NO:246.

In some embodiments, a Plasmodium polypeptide construct described herein comprises a secretory signal (e.g., a secretory signal described herein) and a helper antigen immediately follows the secretory signal.

In some embodiments, a Plasmodium polypeptide construct described herein comprises a helper antigen located at the C-terminus.

In some embodiments, a Plasmodium polypeptide construct described herein comprises a linker between the CSP portion and the helper antigen.

### E. Multimerization Regions

In some embodiments, a Plasmodium polypeptide construct described herein includes one or more multimerization regions (e.g., a heterologous multimerization region). In some embodiments, a heterologous multimerization region comprises a dimerization, trimerization or tetramerization region.

In some embodiments, a multimerization region is one described in WO2017/081082, which is incorporated herein by reference in its entirety (e.g., SEQ ID NOs: 1116-1167, or fragments or variants thereof). Exemplary trimerization and tetramerization regions include, but are not limited to, engineered leucine zippers, fibritin foldon domain from enterobacteria phage T4, GCN4pll, GCN4-pll, and p53.

In some embodiments, a provided Plasmodium polypeptide construct described herein is able to form a trimeric complex. For example, a provided Plasmodium polypeptide construct may comprise a multimerization region allowing formation of a multimeric complex, such as for example a trimeric complex of a Plasmodium polypeptide construct described herein. In some embodiments, a multimerization region allowing formation of a multimeric complex comprises a trimerization region, for example, a trimerization region described herein. In some embodiments, a Plasmodium polypeptide construct includes a T4-fibritin-derived “foldon” trimerization region, for example, to increase its immunogenicity. In some embodiments, a Plasmodium polypeptide construct includes a multimerization region comprising or consisting of an amino acid sequence according to SEQ ID NO: 255.

### F. Linkers

In some embodiments, a Plasmodium polypeptide construct described herein includes one or more linkers. In some embodiments, a linker is or comprises 2, 3, 4, 5, 6, 7, 8, 9, 10 or more amino acids. In some embodiments, a linker is or comprises no more than about 30, 25, 20, 15, 10 or fewer amino acids. A linker can include any amino acid sequence and is not limited to any particular amino acids. In some embodiments, a linker comprises one or more glycine (G) amino acids. In some embodiments, a linker comprises one or more serine(S) amino acids. In some embodiments, a linker includes amino acids selected based on a cleavage predictor to generate highly-cleavable linkers.

In some embodiments, a linker is or comprises S-G₄-S-G₄-S. In some embodiments, a linker is or comprises an amino acid sequence according to SEQ ID NO: 267. In some embodiments, a linker is or comprises an amino acid sequence according to SEQ ID NO: 258. In some embodiments, a linker is has an amino acid sequence according to SEQ ID NO: 261, 267, 258, 276, 279, 270, 282, 264, or 273. In some embodiments, a linker is or comprises a sequence as set forth in WO2017/081082, which is incorporated herein by reference in its entirety (see SEQ ID NOs: 1509-1565, or a fragment or variant thereof).

In some embodiments, a Plasmodium polypeptide construct described herein comprises a linker between a C-terminal region or portion thereof and a transmembrane region. In some embodiments, a Plasmodium polypeptide construct described herein comprises a linker after a minor repeat sequence.

### G. Embodiments of Plasmodium Polypeptide Constructs

In some embodiments, a Plasmodium polypeptide construct described herein includes one or more Plasmodium CSP polypeptide regions or portions thereof as described above. Exemplary combinations of regions are described below.

### Full Length CSP Constructs

In some embodiments, a Plasmodium polypeptide construct described herein includes one or more regions or portions of CSP from Plasmodium falciparum, preferably from Plasmodium falciparum isolate 3D7. In some embodiments, a Plasmodium polypeptide construct described herein includes one or more of a N-terminal region, a N-terminal end region, a junction region, a minor repeat region, a major repeat region and a C-terminal region or corresponding portions thereof of CSP from Plasmodium falciparum, preferably from Plasmodium falciparum isolate 3D7. In some embodiments, the Plasmodium polypeptide construct described herein has the structure: N-terminal region-N-terminal end region-junction region-minor repeat region-major repeat region-C-terminal region, wherein the regions are from CSP from Plasmodium falciparum, preferably from Plasmodium falciparum isolate 3D7. In preferred embodiments, such Plasmodium polypeptide constructs have immediately following the C-terminal region a serine or a serine and a valine. In preferred embodiments, the N-terminal region or portion thereof comprises the amino acid sequence of positions 19 to 80 of SEQ ID NO: 1, or an amino acid sequence having at least 99%, 98%, 97%, 96%, 95%, 90%, 85%, or 80% identity to the amino acid sequence of positions 19 to 80 of SEQ ID NO: 1. In preferred embodiments, the N-terminal end region or portion thereof comprises the amino acid sequence of positions 81 to 92 of SEQ ID NO: 1, or the amino acid sequence of positions 81 to 92 of SEQ ID NO:1 having 1, 2, 3, 4, or 5 amino acid substitutions. In preferred embodiments, the junction region or portion thereof comprises the amino acid sequence of positions 93 to 104 of SEQ ID NO:1, or the amino acid sequence of positions 93 to 104 of SEQ ID NO:1 having 1, 2, 3, 4, or 5 amino acid substitutions. In preferred embodiments, the minor repeat region or portion thereof comprises the amino acid sequence of positions 105 to 128 of SEQ ID NO: 1, or an amino acid sequence having at least 99%, 98%, 97%, 96%, 95%, 90%, 85%, or 80% identity to the amino acid sequence of positions 105 to 128 of SEQ ID NO: 1. In preferred embodiments, the major repeat region or portion thereof comprises the amino acid sequence of positions 129 to 272 of SEQ ID NO:1, or an amino acid sequence having at least 99%, 98%, 97%, 96%, 95%, 90%, 85%, or 80% identity to the amino acid sequence of positions 129 to 272 of SEQ ID NO:1. In preferred embodiments, the C-terminal region or portion thereof comprises the amino acid sequence of positions 273 to 375 of SEQ ID NO:1, or an amino acid sequence having at least 99%, 98%, 97%, 96%, 95%, 90%, 85%, or 80% identity to the amino acid sequence of positions 273 to 375 of SEQ ID NO:1. In preferred embodiments, a Plasmodium polypeptide construct comprises the amino acid sequence of positions 19-375 of SEQ ID NO:1, or an amino acid sequence having at least 99%, 98%, 97%, 96%, 95%, 90%, 85%, or 80% identity to the amino acid sequence of positions 19-375 of SEQ ID NO:1.

Such a Plasmodium polypeptide construct that includes all CSP regions as mentioned before and includes a serine or serine and valine immediately following the C-terminal region is referred to as a full-length CSP construct.

In some embodiments, a Plasmodium polypeptide construct can have the following structure:
full-length CSP construct;
sec-full-length CSP construct;
full-length CSP construct-TMD;
sec-full-length CSP construct-TMD;
Pfsec-full-length CSP construct;
full-length CSP construct-PfTMD;
Pfsec-full-length CSP construct-PfTMD;
HSV-1gDsec-full-length CSP construct;
full-length CSP construct-HSV-fTMD;
HSV-1gDsec-full-length CSP construct-HSV-1TMD;
Pfsec-full-length CSP construct-HSV-fTMD;
HSV-1gDsec-full-length CSP construct-PATMD;
heterologoussec-full-length CSP construct;
full-length CSP construct-heterologousTMD;
heterologoussec-full-length CSP construct-heterologousTMD;
full-length CSP construct-multimerization;
sec-full-length CSP construct-multimerization;
full-length CSP construct-TMD-multimerization;
sec-full-length CSP construct-TMD-multimerization;
Pfsec-full-length CSP construct-multimerization;
full-length CSP construct-PfTMD-multimerization;
Pfsec-full-length CSP construct-PfTMD-multimerization;
HSV-1gDsec-full-length CSP construct-multimerization;
full-length CSP construct-HSV-1TMD-multimerization;
HSV-1gDsec-full-length CSP construct-HSV-1TMD-multimerization;
Pfsec-full-length CSP construct-HSV-fTMD-multimerization;
HSV-1gDsec-full-length CSP construct-PATMD-multimerization;
heterologoussec-full-length CSP construct-multimerization;
full-length CSP construct-heterologousTMD-multimerization; or
heterologoussec-full-length CSP construct-heterologousTMD-multimerization.
   CSP Constructs with Noncontiguous Minor Repeat Regions

In some embodiments, a Plasmodium polypeptide construct described herein includes one or more of a N-terminal end region, a junction region, a minor repeat region, a major repeat region portion and a C-terminal region or corresponding portions thereof of CSP from Plasmodium falciparum, preferably from Plasmodium falciparum isolate 3D7.

In some embodiments, a Plasmodium polypeptide construct described herein has the structure: N-terminal end region-junction region-[minor repeat region-major repeat region portion]ₓ-minor repeat region-C-terminal region, wherein the [minor repeat region-major repeat region portion]repeats x times, and wherein the regions are from CSP from Plasmodium falciparum, preferably from Plasmodium falciparum isolate 3D7. In some embodiments, x is 2 to 5 (i.e., the [minor repeat region-major repeat region portion]repeats 2 to 5 times). In preferred embodiments, such Plasmodium polypeptide constructs have two repeats of a [minor repeat region-major repeat region portion], such that the the Plasmodium polypeptide construct described herein has the structure: N-terminal end region-junction region-minor repeat region-major repeat region portion-minor repeat region-major repeat region portion-minor repeat region-C-terminal region. In preferred embodiments, the N-terminal end region or portion thereof comprises the amino acid sequence of positions 81 to 92 of SEQ ID NO: 1, or the amino acid sequence of positions 81 to 92 of SEQ ID NO:1 having 1, 2, 3, 4, or 5 amino acid substitutions. In preferred embodiments, the junction region includes an R1 region (amino acids 93-97) of SEQ ID NO: 1. In preferred embodiments, the junction region or portion thereof comprises the amino acid sequence of positions 93 to 104 of SEQ ID NO: 1, or the amino acid sequence of positions 93 to 104 of SEQ ID NO: 1 having 1, 2, 3, 4, or 5 amino acid substitutions. In preferred embodiments, a minor repeat region or portion thereof comprises the amino acid sequence of positions 105 to 128 of SEQ ID NO: 1, or an amino acid sequence having at least 99%, 98%, 97%, 96%, 95%, 90%, 85%, or 80% identity to the amino acid sequence of positions 105 to 128 of SEQ ID NO:1. In some embodiments, a major repeat region portion comprises at least four repeats, at least five repeats, at least six repeats, at least seven repeats of the sequence NANP (SEQ ID NO: 147). In preferred embodiments, the major repeat region portion comprises a sequence of NANPNANPNANPNANPNANPNANP (SEQ ID NO: 437). In preferred embodiments, the C-terminal region or portion thereof comprises the amino acid sequence of positions 273 to 375 of SEQ ID NO: 1, or an amino acid sequence having at least 99%, 98%, 97%, 96%, 95%, 90%, 85%, or 80% identity to the amino acid sequence of positions 273 to 375 of SEQ ID NO: 1. A Plasmodium polypeptide construct that includes all CSP regions or corresponding portions thereof as mentioned before and includes noncontiguous minor repeat regions (i.e., minor repeat region-major repeat region portion-minor repeat region-major repeat region portion-minor repeat region) is referred to as a 3×MR CSP construct. In some embodiments, a Plasmodium polypeptide construct can have the following structure:
3×MR CSP construct;
sec-3×MR CSP construct;
3×MR CSP construct-TMD;
sec-3×MR CSP construct-TMD;
Pfsec-3×MR CSP construct;
3×MR CSP construct-PfTMD;
Pfsec-3×MR CSP construct-PfTMD;
HSV-1gDsec-3×MR CSP construct;
3×MR CSP construct-HSV-fTMD;
HSV-1gDsec-3×MR CSP construct-HSV-fTMD;
HSV-1gDsec-3×MR CSP construct-PfTMD;
Pfsec-3×MR CSP construct-HSV-fTMD;
heterologoussec-3×MR CSP construct;
3×MR CSP construct-heterologousTMD; or
heterologoussec-3×MR CSP construct-heterologousTMD.

### N-Terminal Region Deleted CSP Constructs

In some embodiments, a Plasmodium polypeptide construct described herein includes one or more of a N-terminal end region, a junction region, a minor repeat region, a major repeat region and a C-terminal region or corresponding portions thereof of CSP from Plasmodium falciparum, preferably from Plasmodium falciparum isolate 3D7. In some embodiments, a Plasmodium polypeptide construct described herein has the structure: N-terminal end region-junction region-minor repeat region-major repeat region-C-terminal region, wherein the regions are from CSP from Plasmodium falciparum, preferably from Plasmodium falciparum isolate 3D7. In preferred embodiments, such Plasmodium polypeptide constructs have immediately following the C-terminal region a serine or a serine and a valine. In preferred embodiments, the N-terminal end region or portion thereof comprises the amino acid sequence of positions 81 to 92 of SEQ ID NO: 1, or the amino acid sequence of positions 81 to 92 of SEQ ID NO:1 having 1, 2, 3, 4, or 5 amino acid substitutions. In preferred embodiments, the junction region or portion thereof comprises the amino acid sequence of positions 93 to 104 of SEQ ID NO:1, or the amino acid sequence of positions 93 to 104 of SEQ ID NO:1 having 1, 2, 3, 4, or 5 amino acid substitutions. In preferred embodiments, the minor repeat region or portion thereof comprises the amino acid sequence of positions 105 to 128 of SEQ ID NO: 1, or an amino acid sequence having at least 99%, 98%, 97%, 96%, 95%, 90%, 85%, or 80% identity to the amino acid sequence of positions 105 to 128 of SEQ ID NO: 1. In preferred embodiments, the major repeat region or portion thereof comprises the amino acid sequence of positions 129 to 272 of SEQ ID NO:1, or an amino acid sequence having at least 99%, 98%, 97%, 96%, 95%, 90%, 85%, or 80% identity to the amino acid sequence of positions 129 to 272 of SEQ ID NO: 1. In preferred embodiments, the C-terminal region or portion thereof comprises the amino acid sequence of positions 273 to 375 of SEQ ID NO:1, or an amino acid sequence having at least 99%, 98%, 97%, 96%, 95%, 90%, 85%, or 80% identity to the amino acid sequence of positions 273 to 375 of SEQ ID NO:1. Such a Plasmodium polypeptide construct that includes all CSP regions or corresponding portions thereof as mentioned before except the N-terminal region or a portion thereof and includes a serine or serine and valine immediately following the C-terminal region is referred to as a dNT CSP construct. In some embodiments, a Plasmodium polypeptide construct can have the following structure:
dNT CSP construct;
sec-dNT CSP construct;
dNT CSP construct-TMD;
sec-dNT CSP construct-TMD;
Pfsec-dNT CSP construct;
dNT CSP construct-PfTMD;
Pfsec-dNT CSP construct-PfTMD;
HSV-1gDsec-dNT CSP construct;
dNT CSP construct-HSV-fTMD;
HSV-1gDsec-dNT CSP construct-HSV-fTMD;
HSV-1gDsec-dNT CSP construct-PfTMD;
Pfsec-dNT CSP construct-HSV-fTMD;
heterologoussec-dNT CSP construct;
dNT CSP construct-heterologousTMD; or
heterologoussec-dNT CSP construct-heterologousTMD.

### N-Terminal Region and Major Repeat Region Deleted CSP Constructs

In some embodiments, a Plasmodium polypeptide construct described herein includes one or more of a N-terminal end region, a junction region, one or more minor repeat region, and a C-terminal region or corresponding portions thereof of CSP from Plasmodium falciparum, preferably from Plasmodium falciparum isolate 3D7. In some embodiments, a Plasmodium polypeptide construct described herein has the structure: N-terminal end region-junction region-one or more minor repeat region-C-terminal region, wherein the regions are from CSP from Plasmodium falciparum, preferably from Plasmodium falciparum isolate 3D7. In preferred embodiments, such Plasmodium polypeptide constructs have immediately following the C-terminal region a serine or a serine and a valine. In preferred embodiments, the N-terminal end region or portion thereof comprises the amino acid sequence of positions 81 to 92 of SEQ ID NO: 1, or the amino acid sequence of positions 81 to 92 of SEQ ID NO:1 having 1, 2, 3, 4, or 5 amino acid substitutions. In preferred embodiments, the junction region or portion thereof comprises the amino acid sequence of positions 93 to 104 of SEQ ID NO:1, or the amino acid sequence of positions 93 to 104 of SEQ ID NO:1 having 1, 2, 3, 4, or 5 amino acid substitutions. In preferred embodiments, the minor repeat region or portion thereof comprises the amino acid sequence of positions 105 to 128 of SEQ ID NO: 1, or an amino acid sequence having at least 99%, 98%, 97%, 96%, 95%, 90%, 85%, or 80% identity to the amino acid sequence of positions 105 to 128 of SEQ ID NO:1. In preferred embodiments, the C-terminal region or portion thereof comprises the amino acid sequence of positions 273 to 375 of SEQ ID NO:1, or an amino acid sequence having at least 99%, 98%, 97%, 96%, 95%, 90%, 85%, or 80% identity to the amino acid sequence of positions 273 to 375 of SEQ ID NO:1. In some embodiments, such Plasmodium polypeptide constructs have more than one minor repeat region, such as three minor repeat regions. Such a Plasmodium polypeptide construct that includes all CSP regions or corresponding portions thereof as mentioned before except the N-terminal region and the major repeat region or corresponding portions thereof, has one or more minor repeat region and includes a serine or serine and valine immediately following the C-terminal region is referred to as a dNT-dmajor CSP construct. In some embodiments, a Plasmodium polypeptide construct can have the following structure:
dNT-dmajor CSP construct;
sec-dNT-dmajor CSP construct;
dNT-dmajor CSP construct-TMD;
sec-dNT-dmajor CSP construct-TMD;
Pfsec-dNT-dmajor CSP construct;
dNT-dmajor CSP construct-PfTMD;
Pfsec-dNT-dmajor CSP construct-PTMD;
HSV-1gDsec-dNT-dmajor CSP construct;
dNT-dmajor CSP construct-HSV-fTMD;
HSV-1gDsec-dNT-dmajor CSP construct-HSV-fTMD;
HSV-1gDsec-dNT-dmajor CSP construct-PfTMD;
Pfsec-dNT-dmajor CSP construct-HSV-fTMD;
heterologoussec-dNT-dmajor CSP construct;
dNT-dmajor CSP construct-heterologousTMD;
heterologoussec-dNT-dmajor CSP construct-heterologousTMD;
dNT-dmajor CSP construct-helper antigen;
sec-dNT-dmajor CSP construct-helper antigen;
dNT-dmajor CSP construct-TMD-helper antigen;
sec-dNT-dmajor CSP construct-TMD-helper antigen;
Pfsec-dNT-dmajor CSP construct-helper antigen;
dNT-dmajor CSP construct-PfTMD-helper antigen;
Pfsec-dNT-dmajor CSP construct-PfTMD-helper antigen;
HSV-1gDsec-dNT-dmajor CSP construct-helper antigen;
dNT-dmajor CSP construct-HSV-fTMD-helper antigen;
HSV-1gDsec-dNT-dmajor CSP construct-HSV-fTMD-helper antigen;
HSV-1gDsec-dNT-dmajor CSP construct-PTMD-helper antigen;
Pfsec-dNT-dmajor CSP construct-HSV-fTMD-helper antigen;
heterologoussec-dNT-dmajor CSP construct-helper antigen;
dNT-dmajor CSP construct-heterologousTMD-helper antigen; or
heterologoussec-dNT-dmajor CSP construct-heterologousTMD-helper antigen.

### N-Terminal Domain Deleted CSP Constructs

In some embodiments, a Plasmodium polypeptide construct described herein includes one or more of a junction region, one or more minor repeat regions, a major repeat region and a C-terminal region or corresponding portions thereof of CSP from Plasmodium falciparum, preferably from Plasmodium falciparum isolate 3D7. In some embodiments, a Plasmodium polypeptide construct described herein has the structure: junction region-one or more minor repeat region-major repeat region-C-terminal region, wherein the regions are from CSP from Plasmodium falciparum, preferably from Plasmodium falciparum isolate 3D7. In preferred embodiments, such Plasmodium polypeptide constructs have immediately following the C-terminal region a serine or a serine and a valine. In preferred embodiments, the junction region or portion thereof comprises the amino acid sequence of positions 93 to 104 of SEQ ID NO:1, or the amino acid sequence of positions 93 to 104 of SEQ ID NO:1 having 1, 2, 3, 4, or 5 amino acid substitutions. In preferred embodiments, the minor repeat region or portion thereof comprises the amino acid sequence of positions 105 to 128 of SEQ ID NO: 1, or an amino acid sequence having at least 99%, 98%, 97%, 96%, 95%, 90%, 85%, or 80% identity to the amino acid sequence of positions 105 to 128 of SEQ ID NO:1. In preferred embodiments, the major repeat region or portion thereof comprises the amino acid sequence of positions 129 to 272 of SEQ ID NO:1, or an amino acid sequence having at least 99%, 98%, 97%, 96%, 95%, 90%, 85%, or 80% identity to the amino acid sequence of positions 129 to 272 of SEQ ID NO:1. In preferred embodiments, the C-terminal region or portion thereof comprises the amino acid sequence of positions 273 to 375 of SEQ ID NO:1, or an amino acid sequence having at least 99%, 98%, 97%, 96%, 95%, 90%, 85%, or 80% identity to the amino acid sequence of positions 273 to 375 of SEQ ID NO:1. In some embodiments, such Plasmodium polypeptide constructs have more than one minor repeat region, such as three minor repeat regions. Such a Plasmodium polypeptide construct that includes all CSP regions or corresponding portions thereof as mentioned before except the N-terminal domain (i.e. exclude the N-terminal region and the N-terminal end region) or a portion thereof, has one or more minor repeat regions and includes a serine or serine and valine immediately following the C-terminal region is referred to as a dND CSP construct. In some embodiments, a Plasmodium polypeptide construct construct can have the following structure:
dND CSP construct;
sec-dND CSP construct;
dND CSP construct-TMD;
sec-dND CSP construct-TMD;
Pfsec-dND CSP construct;
dND CSP construct-PfTMD;
Pfsec-dND CSP construct-PfTMD;
HSV-1gDsec-dND CSP construct;
dND CSP construct-HSV-1TMD;
HSV-1gDsec-dND CSP construct-HSV-1TMD;
HSV-1gDsec-dND CSP construct-PfTMD;
Pfsec-dND CSP construct-HSV-1TMD;
heterologoussec-dND CSP construct;
dND CSP construct-heterologousTMD; or
heterologoussec-dND CSP construct-heterologousTMD.

### N-Terminal Domain and Major Repeat Region Deleted CSP Constructs

In some embodiments, a Plasmodium polypeptide construct described herein includes a junction region, one or more minor repeat region, and a C-terminal region or corresponding portions thereof of CSP from Plasmodium falciparum, preferably from Plasmodium falciparum isolate 3D7. In some embodiments, a Plasmodium polypeptide construct described herein has the structure: junction region-one or more minor repeat region-C-terminal region, wherein the regions are from CSP from Plasmodium falciparum, preferably from Plasmodium falciparum isolate 3D7. In preferred embodiments, such Plasmodium polypeptide constructs have immediately following the C-terminal region a serine or a serine and a valine. In preferred embodiments, the junction region or portion thereof comprises the amino acid sequence of positions 93 to 104 of SEQ ID NO:1, or the amino acid sequence of positions 93 to 104 of SEQ ID NO: 1 having 1, 2, 3, 4, or 5 amino acid substitutions. In preferred embodiments, the minor repeat region or portion thereof comprises the amino acid sequence of positions 105 to 128 of SEQ ID NO: 1, or an amino acid sequence having at least 99%, 98%, 97%, 96%, 95%, 90%, 85%, or 80% identity to the amino acid sequence of positions 105 to 128 of SEQ ID NO:1. In preferred embodiments, the C-terminal region or portion thereof comprises the amino acid sequence of positions 273 to 375 of SEQ ID NO:1, or an amino acid sequence having at least 99%, 98%, 97%, 96%, 95%, 90%, 85%, or 80% identity to the amino acid sequence of positions 273 to 375 of SEQ ID NO:1. In some embodiments, such Plasmodium polypeptide constructs have more than one minor repeat region, such as three minor repeat regions. Such a Plasmodium polypeptide construct that includes all CSP regions or corresponding portions thereof as mentioned before except the N-terminal domain (i.e. exclude the N-terminal region and the N-terminal end region) and the major repeat region or corresponding portions thereof, has one or more minor repeat region and includes a serine or serine and valine immediately following the C-terminal region is referred to as a dND-dmajor CSP construct. In some embodiments, a Plasmodium polypeptide construct can have the following structure:
dND-dmajor CSP construct;
sec-dND-dmajor CSP construct;
dND-dmajor CSP construct-TMD;
sec-dND-dmajor CSP construct-TMD;
Pfsec-dND-dmajor CSP construct;
dND-dmajor CSP construct-PfTMD;
Pfsec-dND-dmajor CSP construct-PfTMD;
HSV-1gDsec-dND-dmajor CSP construct;
dND-dmajor CSP construct-HSV-1TMD;
HSV-1gDsec-dND-dmajor CSP construct-HSV-1TMD;
HSV-1gDsec-dND-dmajor CSP construct-PfTMD;
Pfsec-dND-dmajor CSP construct-HSV-1TMD;
heterologoussec-dND-dmajor CSP construct;
dND-dmajor CSP construct-heterologousTMD; or
heterologoussec-dND-dmajor CSP construct-heterologousTMD.
   N-Terminal Domain and Major Repeat Region Deleted CSP Constructs with Junction Region Variants or Portions

In some embodiments, a Plasmodium polypeptide construct described herein includes a junction region variant or junction region portion, one or more minor repeat regions, and a C-terminal region or corresponding portions thereof of CSP from Plasmodium falciparum, preferably from Plasmodium falciparum isolate 3D7. In some embodiments, a Plasmodium polypeptide construct described herein has the structure: junction region variant or junction region portion-one or more minor repeat region-C-terminal region, wherein the regions are from CSP from Plasmodium falciparum, preferably from Plasmodium falciparum isolate 3D7. In preferred embodiments, such Plasmodium polypeptide constructs have immediately following the C-terminal region a serine or a serine and a valine. In preferred embodiments, the junction region variant or portion thereof comprises the amino acid sequence of positions 93 to 104 of SEQ ID NO:1 having 1, 2, 3, 4, or 5 amino acid substitutions. In preferred embodiments, the junction region variant or portion thereof comprises the amino acid sequence of positions 93 to 104 of SEQ ID NO:1 having a K93A mutation, an L94A mutation, or both. In preferred embodiments, the junction region portion consists of a portion of the amino acid sequence of positions 93 to 104 of SEQ ID NO:1. In preferred embodiments, the junction region portion consists of the amino acid sequence of positions 97 to 104 of SEQ ID NO:1. In preferred embodiments, the junction region portion comprises or consists of the amino acid sequence of positions 98 to 104 of SEQ ID NO:1. In preferred embodiments, the minor repeat region or portion thereof comprises the amino acid sequence of positions 105 to 128 of SEQ ID NO: 1, or an amino acid sequence having at least 99%, 98%, 97%, 96%, 95%, 90%, 85%, or 80% identity to the amino acid sequence of positions 105 to 128 of SEQ ID NO:1. In preferred embodiments, the C-terminal region or portion thereof comprises the amino acid sequence of positions 273 to 375 of SEQ ID NO: 1, or an amino acid sequence having at least 99%, 98%, 97%, 96%, 95%, 90%, 85%, or 80% identity to the amino acid sequence of positions 273 to 375 of SEQ ID NO:1. In some embodiments, such Plasmodium polypeptide constructs have more than one minor repeat region, such as three minor repeat regions. Such a Plasmodium polypeptide construct that includes all CSP regions or corresponding portions thereof as mentioned before except the N-terminal domain (i.e. exclude the N-terminal region and the N-terminal end region) and the major repeat region or corresponding portions thereof, has a junction region variant or junction region portion, has one or more minor repeat region and includes a serine or serine and valine immediately following the C-terminal region is referred to as a dND-dmajor-modJ CSP construct. In some embodiments, a Plasmodium polypeptide construct can have the following structure:
dND-dmajor-modJ CSP construct;
sec-dND-dmajor-modJ CSP construct;
dND-dmajor-modJ CSP construct-TMD;
sec-dND-dmajor-modJ CSP construct-TMD;
Pfsec-dND-dmajor-modJ CSP construct;
dND-dmajor-modJ CSP construct-PfTMD;
Pfsec-dND-dmajor-modJ CSP construct-PTMD;
HSV-1gDsec-dND-dmajor-modJ CSP construct;
dND-dmajor-modJ CSP construct-HSV-1TMD;
HSV-1gDsec-dND-dmajor-modJ CSP construct-HSV-1TMD;
HSV-1gDsec-dND-dmajor-modJ CSP construct-PfTMD;
Pfsec-dND-dmajor-modJ CSP construct-HSV-1TMD;
heterologoussec-dND-dmajor-modJ CSP construct;
dND-dmajor-modJ CSP construct-heterologousTMD; or
heterologoussec-dND-dmajor-modJ CSP construct-heterologousTMD.

### Major Repeat Region Portion and C-Terminal Region Containing CSP Constructs

In some embodiments, a Plasmodium polypeptide construct described herein includes a major repeat region portion and a C-terminal region or corresponding portions thereof of CSP from Plasmodium falciparum, preferably from Plasmodium falciparum isolate 3D7. In some embodiments, a Plasmodium polypeptide construct described herein has the structure: major repeat region portion-C-terminal region, wherein the regions are from CSP from Plasmodium falciparum, preferably from Plasmodium falciparum isolate 3D7. In preferred embodiments, such Plasmodium polypeptide constructs have immediately following the C-terminal resion a serine or a serine and a valine. In some embodiments, such Plasmodium polypeptide constructs have between 2 and 35 repeats of the amino acid sequence NANP (SEQ ID NO: 147), preferably 18 repeats of the amino acid sequence NANP (SEQ ID NO: 147) as a major repeat portion. In preferred embodiments, the C-terminal region or portion thereof comprises the amino acid sequence of positions 273 to 375 of SEQ ID NO:1, or an amino acid sequence having at least 99%, 98%, 97%, 96%, 95%, 90%, 85%, or 80% identity to the amino acid sequence of positions 273 to 375 of SEQ ID NO:1. Such a Plasmodium polypeptide construct that includes only a portion of the CSP major repeat region, a C-terminal region and includes a serine or serine and valine immediately following the C-terminal region, or that includes corresponding portions thereof as mentioned before, is referred to as a pmajor-CT CSP construct. In some embodiments, a Plasmodium polypeptide construct can have the following structure:
pmajor-CT CSP construct;
sec-pmajor-CT CSP construct;
pmajor-CT CSP construct-TMD;
sec-pmajor-CT CSP construct-TMD;
Pfsec-pmajor-CT CSP construct;
pmajor-CT CSP construct-PTMD;
Pfsec-pmajor-CT CSP construct-PfTMD;
HSV-1gDsec-pmajor-CT CSP construct;
pmajor-CT CSP construct-HSV-1TMD;
HSV-1gDsec-pmajor-CT CSP construct-HSV-1TMD;
HSV-1gDsec-pmajor-CT CSP construct-PfTMD;
Pfsec-pmajor-CT CSP construct-HSV-1TMD;
heterologoussec-pmajor-CT CSP construct;
pmajor-CT CSP construct-heterologousTMD; or
heterologoussec-pmajor-CT CSP construct-heterologousTMD.
   N-Terminal and C-Terminal Deleted CSP Constructs with Noncontiguous Minor Repeat Regions

In some embodiments, a Plasmodium polypeptide construct described herein includes one or more of an N-terminal end region, a junction region, a minor repeat region, a major repeat region portion and a C-terminal region or corresponding portions thereof of CSP from Plasmodium falciparum, preferably from Plasmodium falciparum isolate 3D7.

In some embodiments, a Plasmodium polypeptide construct described herein has the structure: [junction region-minor repeat region-major repeat region portion]ₓ, wherein the [junction region-minor repeat region-major repeat region portion]repeats x times, and wherein the regions are from CSP from Plasmodium falciparum, preferably from Plasmodium falciparum isolate 3D7. In some embodiments, x is 2 to 5 (i.e., the [junction region-minor repeat region-major repeat region portion]repeats 2 to 5 times). In preferred embodiments, such Plasmodium polypeptide constructs have three repeats of a [junction region-minor repeat region-major repeat region portion], such that the the Plasmodium polypeptide construct described herein has the structure: junction region-minor repeat region-major repeat region portion-junction region-minor repeat region-major repeat region portion-junction region-minor repeat region-C-terminal region. In preferred embodiments, the N-terminal end region or portion thereof comprises the amino acid sequence of positions 81 to 92 of SEQ ID NO: 1, or the amino acid sequence of positions 81 to 92 of SEQ ID NO:1 having 1, 2, 3, 4, or 5 amino acid substitutions. In preferred embodiments, the junction region includes an R1 region (amino acids 93-97) of SEQ ID NO:1. In preferred embodiments, the junction region repeats twice. In preferred embodiments, the junction region or portion thereof comprises a 2× repeat of the amino acid sequence of positions 93 to 104 of SEQ ID NO:1, or the amino acid sequence of positions 93 to 104 of SEQ ID NO:1 having 1, 2, 3, 4, or 5 amino acid substitutions. In preferred embodiments, a minor repeat region or portion thereof comprises the amino acid sequence of positions 105 to 128 of SEQ ID NO: 1, or an amino acid sequence having at least 99%, 98%, 97%, 96%, 95%, 90%, 85%, or 80% identity to the amino acid sequence of positions 105 to 128 of SEQ ID NO:1. In some embodiments, a major repeat region portion comprises at least four repeats, at least five repeats, at least six repeats, at least seven repeats of the sequence NANP (SEQ ID NO: 147). In preferred embodiments, the major repeat region portion comprises a sequence of NANPNANPNANPNANPNANPNANP (SEQ ID NO: 437). In some embodiments, the Plasmodium construct further comprises one or more linkers (e.g., gly-ser linkers). In some embodiments, the Plasmodium construct further comprises a linker (e.g., a gly-ser linker) after each major repeat region portion sequence. In some embodiments, the Plasmodium construct comprises a linker (e.g., a gly-ser linker) after the last partial major repeat sequence. In some embodiments, a linker has the amino acid sequence of SEQ ID NO: 258. A Plasmodium polypeptide construct that includes all CSP regions or corresponding portions thereof as mentioned before and includes three repeats of a [junction region-minor repeat region-major repeat region portion] is referred to as a 3×MR-dNC CSP construct. In some embodiments, a Plasmodium polypeptide construct can have the following structure:
3×MR-dNC CSP construct;
sec-3×MR-dNC CSP construct;
3×MR-dNC CSP construct-TMD;
sec-3×MR-dNC CSP construct-TMD;
Pfsec-3×MR-dNC CSP construct;
3×MR-dNC CSP construct-PfTMD;
Pfsec-3×MR-dNC CSP construct-PfTMD;
HSV-1gDsec-3×MR-dNC CSP construct;
3×MR-dNC CSP construct-HSV-1TMD;
HSV-1gDsec-3×MR-dNC CSP construct-HSV-1TMD;
HSV-1gDsec-3×MR-dNC CSP construct-PfTMD;
Pfsec-3×MR-dNC CSP construct-HSV-1TMD;
heterologoussec-3×MR-dNC CSP construct;
3×MR-dNC CSP construct-heterologousTMD; or
heterologoussec-3×MR-dNC CSP construct-heterologousTMD.

### H. Exemplary Construct Sequences

In some embodiments, a Plasmodium polypeptide construct described herein has an amino acid sequence provided by a SEQ ID NO listed in Table 5, and/or is encoded by a nucleotide sequence provided by a SEQ ID NO listed in Table 6A or Table 6B. As used herein, an “ERMA” construct is an “RNA construct,” and for example, “ERMA 1” corresponds to “RNA Construct 1,” “ERMA 2” corresponds to “RNA Construct 2,” etc. in Tables 5, 6A, and 6B below.

TABLE 5
Exemplary Amino Acid Sequences for
RNA Constructs as Described Herein

| RNA Construct | SEQ ID NO. |
|---|---|
| 1 | 3 |
| 2 | 6 |
| 3 | 9 |
| 4 | 12 |
| 5 | 15 |
| 6 | 18 |
| 7 | 21 |
| 8 | 24 |
| 9 | 27 |
| 22 | 30 |
| 23 | 33 |
| 24 | 36 |
| 25 | 39 |
| 26 | 42 |
| 27 | 45 |
| 28 | 48 |
| 29 | 51 |
| 30 | 54 |
| 31 | 57 |
| 32 | 60 |
| 33 | 63 |
| 34 | 66 |
| 35 | 69 |
| 36 | 72 |
| 37 | 75 |
| 38 | 78 |
| 39 | 81 |
| 40 | 84 |
| 41 | 87 |
| 42 | 90 |
| 43 | 419 |
| 44 | 422 |
| 45 | 93 |
| 59 | 96 |
| 60 | 99 |
| 87 | 432 |
| 88 | 433 |
| 91 | 434 |
| 100 | 435 |
| 104 | 436 |

TABLE 6A
Exemplary Nucleotide Sequences for Certain
DNA Constructs as Described Herein

| DNA Construct | SEQ ID NO: |
|---|---|
| 1 | 2 |
| 2 | 5 |
| 4 | 11 |
| 5 | 14 |
| 6 | 17 |
| 7 | 20 |
| 8 | 23 |
| 9 | 26 |
| 22 | 29 |
| 23 | 32 |
| 24 | 35 |
| 25 | 38 |
| 26 | 41 |
| 27 | 44 |
| 28 | 47 |
| 29 | 50 |
| 30 | 53 |
| 31 | 56 |
| 32 | 59 |
| 33 | 62 |
| 34 | 65 |
| 35 | 68 |
| 36 | 71 |
| 37 | 74 |
| 38 | 77 |
| 39 | 80 |
| 40 | 83 |
| 41 | 86 |
| 42 | 89 |
| 43 | 418 |
| 44 | 421 |
| 45 | 92 |
| 59 | 95 |
| 60 | 98 |
| 87 | 441 |
| 88 | 443 |
| 91 | 445 |
| 100 | 447 |
| 104 | 449 |

TABLE 6B
Exemplary Nucleotide Sequences for Certain
RNA Constructs as Described Herein

| RNA Construct | SEQ ID NO: |
|---|---|
| 1 | 4 |
| 2 | 7 |
| 4 | 13 |
| 5 | 16 |
| 6 | 19 |
| 7 | 22 |
| 8 | 25 |
| 9 | 28 |
| 22 | 31 |
| 23 | 34 |
| 24 | 37 |
| 25 | 40 |
| 26 | 43 |
| 27 | 46 |
| 28 | 49 |
| 29 | 52 |
| 30 | 55 |
| 31 | 58 |
| 32 | 61 |
| 33 | 64 |
| 34 | 67 |
| 35 | 70 |
| 36 | 73 |
| 37 | 76 |
| 38 | 79 |
| 39 | 82 |
| 40 | 85 |
| 41 | 88 |
| 42 | 91 |
| 43 | 420 |
| 44 | 423 |
| 45 | 94 |
| 59 | 97 |
| 60 | 100 |
| 87 | 442 |
| 88 | 444 |
| 91 | 446 |
| 100 | 448 |
| 104 | 450 |

### III. Polyribonucleotides

### A. Exemplary Polyribonucleotides Features

Polyribonucleotides described herein encode one or more Plasmodium polypeptide constructs described herein. In some embodiments, polyribonucleotides described herein can comprise a nucleotide sequence that encodes a 5′UTR of interest and/or a 3′ UTR of interest. In some embodiments, polynucleotides described herein can comprise a nucleotide sequence that encodes a polyA tail. In some embodiments, polyribonucleotides described herein may comprise a 5′ cap, which may be incorporated during transcription, or joined to a polyribonucleotide post-transcription.

### 1. 5′ Cap

A structural feature of mRNAs is cap structure at five-prime end (5′). Natural eukaryotic mRNA comprises a 7-methylguanosine cap linked to the mRNA via a 5′ to 5′-triphosphate bridge resulting in cap0 structure (m7GpppN). In most eukaryotic mRNA and some viral mRNA, further modifications can occur at the 2′-hydroxy-group (2′—OH) (e.g., the 2′-hydroxyl group may be methylated to form 2′—O-Me) of the first and subsequent nucleotides producing “cap1” and “cap2” five-prime ends, respectively). Diamond, et al., (2014) Cytokine & growth Factor Reviews, 25:543-550, which is incorporated herein by reference in its entirety, reported that cap0-mRNA cannot be translated as efficiently as cap1-mRNA in which the role of 2′—O-Me in the penultimate position at the mRNA 5′ end is determinant. Lack of the 2′—O-met has been shown to trigger innate immunity and activate IFN response. Daffis, et al. (2010) Nature, 468:452-456; and Züst et al. (2011) Nature Immunology, 12:137-143, each of which is incorporated herein by reference in its entirety.

RNA capping is well researched and is described, e.g., in Decroly E et al. (2012) Nature Reviews 10:51-65; and in Ramanathan A. et al., (2016) Nucleic Acids Res; 44 (16): 7511-7526, the entire contents of each of which is hereby incorporated by reference. For example, in some embodiments, a 5′-cap structure which may be suitable in the context of the present invention is a cap0 (methylation of the first nucleobase, e.g., m7GpppN), cap1 (additional methylation of the ribose of the adjacent nucleotide of m7GpppN), cap2 (additional methylation of the ribose of the 2nd nucleotide downstream of the m7GpppN), cap3 (additional methylation of the ribose of the 3rd nucleotide downstream of the m7GpppN), cap4 (additional methylation of the ribose of the 4th nucleotide downstream of the m7GpppN), ARCA (“anti-reverse cap analogue”), modified ARCA (e.g. phosphothioate modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

The term “5′-cap” as used herein refers to a structure found on the 5′-end of an RNA, e.g., mRNA, and generally includes a guanosine nucleotide connected to an RNA, e.g., mRNA, via a 5′- to 5′-triphosphate linkage (also referred to as Gppp or G (5′) ppp (5′)). In some embodiments, a guanosine nucleoside included in a 5′ cap may be modified, for example, by methylation at one or more positions (e.g., at the 7-position) on a base (guanine), and/or by methylation at one or more positions of a ribose. In some embodiments, a guanosine nucleoside included in a 5′ cap comprises a 3′-O methylation at a ribose (3′-OMeG). In some embodiments, a guanosine nucleoside included in a 5′ cap comprises methylation at the 7-position of guanine (m7G). In some embodiments, a guanosine nucleoside included in a 5′ cap comprises methylation at the 7-position of guanine and a 3′ O methylation at a ribose (m7 (3′-OMeG)). It will be understood that the notation used in the above paragraph, e.g., “(m₂^{7,3′—O}) G” or “m7 (3′-OMeG)”, applies to other structures described herein.

In some embodiments, providing an RNA with a 5′-cap disclosed herein may be achieved by in vitro transcription, in which a 5′-cap is co-transcriptionally expressed into an RNA strand, or may be attached to an RNA post-transcriptionally using capping enzymes. In some embodiments, co-transcriptional capping with a cap disclosed improves the capping efficiency of an RNA compared to co-transcriptional capping with an appropriate reference comparator. In some embodiments, improving capping efficiency can increase a translation efficiency and/or translation rate of an RNA, and/or increase expression of an encoded polypeptide. In some embodiments, alterations to polynucleotides generates a non-hydrolyzable cap structure which can, for example, prevent decapping and increase RNA half-life.

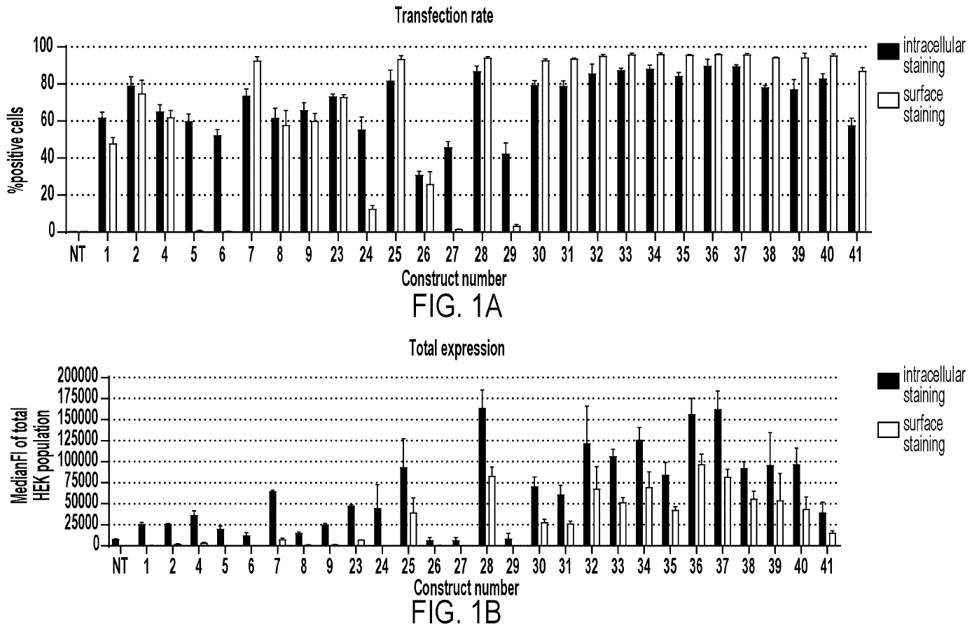

In some embodiments, a utilized 5′ caps is a cap0, a cap1, or cap2 structure. See, e.g., FIG. 1 of Ramanathan A et al., and FIG. 1 of Decroly E et al., each of which is incorporated herein by reference in its entirety. In some embodiments, an RNA described herein comprises a cap1 structure. In some embodiments, an RNA described herein comprises a cap2.

In some embodiments, an RNA described herein comprises a cap0 structure. In some embodiments, a cap0 structure comprises a guanosine nucleoside methylated at the 7-position of guanine ((m7)G). In some embodiments, such a cap0 structure is connected to an RNA via a 5′- to 5′-triphosphate linkage and is also referred to herein as (m7)Gppp. In some embodiments, a cap0 structure comprises a guanosine nucleoside methylated at the 2′-position of the ribose of guanosine. In some embodiments, a cap0 structure comprises a guanosine nucleoside methylated at the 3′-position of the ribose of guanosine. In some embodiments, a guanosine nucleoside included in a 5′ cap comprises methylation at the 7-position of guanine and at the 2′-position of the ribose ((m₂^{7,2′—O})G). In some embodiments, a guanosine nucleoside included in a 5′ cap comprises methylation at the 7-position of guanine and at the 2′-position of the ribose (m₂^{7,3-O})G).

In some embodiments, a cap1 structure comprises a guanosine nucleoside methylated at the 7-position of guanine ((m⁷)G) and optionally methylated at the 2′ or 3′ position of the ribose, and a 2′ methylated first nucleotide in an RNA ((m^{2-O}) N1). In some embodiments, a cap1 structure comprises a guanosine nucleoside methylated at the 7-position of guanine ((m⁷)G) and the 3′ position of the ribose, and a 2′ methylated first nucleotide in an RNA ((m^{2-O}) N1). In some embodiments, a cap1 structure is connected to an RNA via a 5′- to 5′-triphosphate linkage and is also referred to herein as, e.g., ((m⁷)Gppp(^{2′-O}) N1) or (m₂^{7,3′—O})Gppp(^{2-O}) N1), wherein N1 is as defined and described herein. In some embodiments, a cap1 structure comprises a second nucleotide, N2, which is at position 2 and is chosen from A, G, C, or U, e.g., (m⁷)Gppp(^{2-O}) N1PN2 or (m₂^{7,3′—O})Gppp(^{2-O}) N1PN2, wherein each of N1 and N2 is as defined and described herein.

In some embodiments, a cap2 structure comprises a guanosine nucleoside methylated at the 7-position of guanine ((m⁷)G) and optionally methylated at the 2′ or 3′ position of the ribose, and a 2′-O methylated first and second nucleotides in an RNA ((m^{2-O}) N1p (m^{2′—O}) N2). In some embodiments, a cap2 structure comprises a guanosine nucleoside methylated at the 7-position of guanine ((m⁷)G) and the 3′ position of the ribose, and a 2′-O methylated first and second nucleotide in an RNA. In some embodiments, a cap2 structure is connected to an RNA via a 5′- to 5′-triphosphate linkage and is also referred to herein as, e.g., ((m⁷)Gppp(^{2-O}) N1p (^{2-O}) N2) or (m₂^{7,3′—O})Gppp(^{2-O}) N1p (^{2-O}) N2), wherein each of N1 and N2 is as defined and described herein.

In some embodiments, the 5′ cap is a dinucleotide cap structure. In some embodiments, the 5′ cap is a dinucleotide cap structure comprising N1, wherein N1 is as defined and described herein. In some embodiments, the 5′ cap is a dinucleotide cap G*N1, wherein N1 is as defined above and herein, and G* comprises a structure of formula (I):

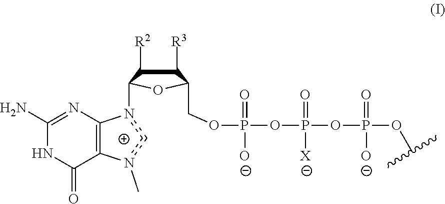

or a salt thereof,
wherein each R² and R³ is —OH or —OCH₃; and X is O or S.

In some embodiments, R² is —OH. In some embodiments, R² is —OCH₃. In some embodiments, R³ is —OH. In some embodiments, R³ is —OCH₃. In some embodiments, R² is —OH and R³ is —OH. In some embodiments, R² is —OH and R³ is-CH₃. In some embodiments, R² is-CH₃ and R³ is —OH. In some embodiments, R² is-CH₃ and R³ is-CH₃.

In some embodiments, X is O. In some embodiments, X is S.

In some embodiments, the 5′ cap is a dinucleotide cap0 structure (e.g., (m⁷)GpppN1, (m₂^{7,2′—O}) GpppN₁, (m₂^{7,3-O})GpppN₁, (m⁷) GppSpN₁, (m₂^{7,2-O}) GppSpN₁, or (m₂^{7,3-O}) GppSpN₁), wherein N1 is as defined and described herein. In some embodiments, the 5′ cap is a dinucleotide cap0 structure (e.g., (m⁷)GpppN₁, (m₂^{7,2-O}) GpppN₁, (m₂^{7,3-O})GpppN₁, (m⁷) GppSpN₁, (m₂^{7,2-O}) GppSpN₁, or (m₂^{7,3-O}) GppSpN₁), wherein N1 is G. In some embodiments, the 5′ cap is a dinucleotide cap0 structure (e.g., (m⁷)GpppN₁, (m₂^{7,2-O})GpppN₁, (m₂^{7,3′—O})GpppN₁, (m⁷) GppSpN₁, (m₂^{7,2′-O}) GppSpN₁, or (m₂⁷³-0) GppSpN₁), wherein N1 is A, U, or C. In some embodiments, the 5′ cap is a dinucleotide cap1 structure (e.g., (m⁷)Gppp(m^{2-O}) N₁, (m₂^{7,2-O})Gppp(m^{2-O}) N₁, (m₂^{7,3-O})Gppp(m^{2-O}) N₁, (m⁷) GppSp (m^{2′-O}) N₁, (m₂^{7,2-O}) GppSp (m^{2-O}) N₁, or (m₂^{7,3-O}) GppSp (m^{2′-O}) N₁), wherein N₁ is as defined and described herein. In some embodiments, the 5′ cap is selected from the group consisting of (m⁷)GpppG (“cap0”), (m⁷) Gpp (m^{2-O}) G (“Ecap1”), (m₂^{7,3-O})GpppG (“ARCA” or “D1”), and (m₂^{7,2-O}) GppSpG (“beta-S-ARCA”). In some embodiments, the 5′ cap is (m⁷)GpppG (“Ecap0”), having a structure:

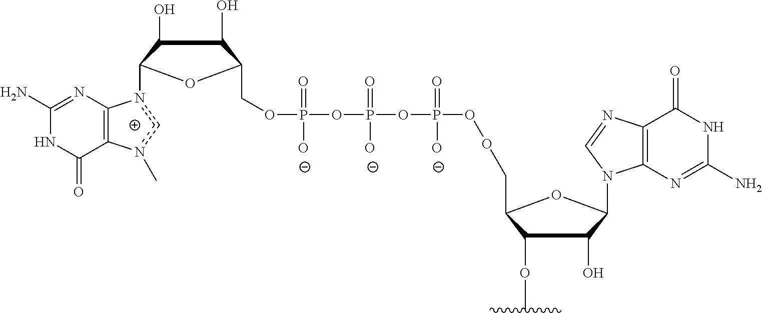

or a salt thereof.

In some embodiments, the 5′ cap is (m⁷)Gppp(m^{2-O}) G (“Ecap1”), having a structure:

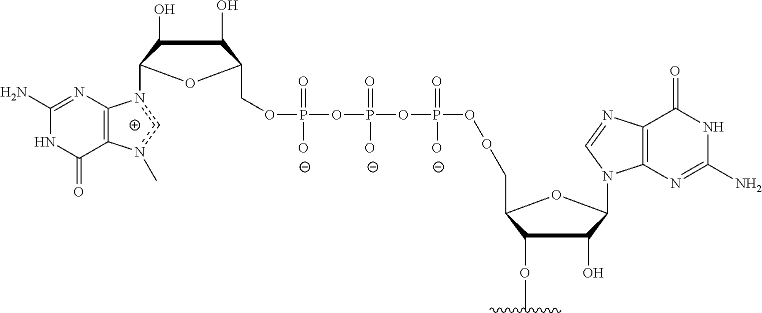

or a salt thereof.

In some embodiments, the 5′ cap is (m₂^{7,3-O})GpppG (“ARCA” or “D1”), having a structure:

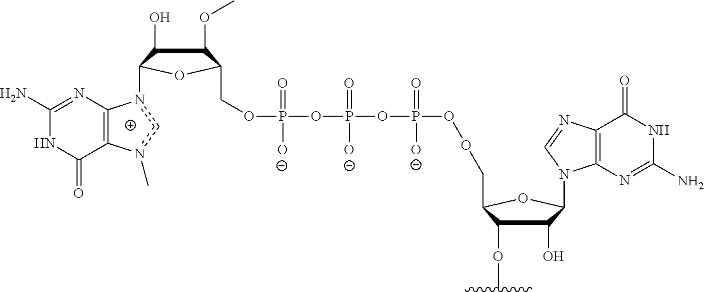

or a salt thereof.

In some embodiments, the 5′ cap is (m₂^{7,2-O}) GppSpG (“beta-S-ARCA”), having a structure:

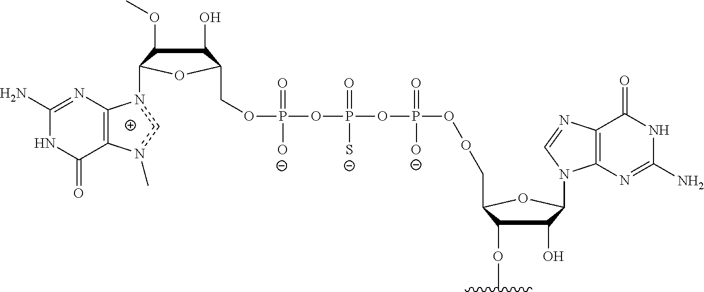

or a salt thereof.

In some embodiments, the 5′ cap is a trinucleotide cap structure. In some embodiments, the 5′ cap is a trinucleotide cap structure comprising N₁pN₂, wherein N₁ and N₂ are as defined and described herein. In some embodiments, the 5′ cap is a dinucleotide cap G*N₁pN₂, wherein N₁ and N₂ are as defined above and herein, and G* comprises a structure of formula (I):

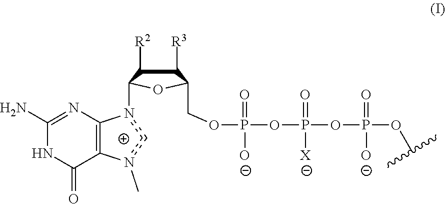

or a salt thereof, wherein R², R³, and X are as defined and described herein.

In some embodiments, the 5′ cap is a trinucleotide cap0 structure (e.g. (m⁷)GpppN₁PN2, (m₂^{7,2′—O}) GpppN₁pN₂, or (m₂^{7,3′-O})GpppN₁pN₂), wherein N₁ and N₂ are as defined and described herein). In some embodiments, the 5′ cap is a trinucleotide cap1 structure (e.g., (m⁷)Gppp(m^{2-O}) N₁pN₂, (m₂^{7,2′-O})Gppp(m²-°) N₁pN₂, (m₂^{7,3-O})Gppp(m^{2-O}) N₁pN₂), wherein N₁ and N₂ are as defined and described herein. In some embodiments, the 5′ cap is a trinucleotide cap2 structure (e.g., (m⁷)Gppp(m^{2-O}) Nip (m^{2-O}) N₂, (m₂^{7,2-O})Gppp(m^{2′-}) N₁p (m^{2-O}) N₂, (m₂^{7,3-O}) Gpp (m^{2-O}) N₁p (m^{2-O}) N₂), wherein N₁ and Ny are as defined and described herein. In some embodiments, the 5′ cap is selected from the group consisting of (m₂^{7,3-O})Gppp(m^{2-O}) ApG (“CleanCap AG”, “CC413”), (m₂^{7,3-O})Gppp(m^{2-O}) GpG (“CleanCap GG”), (m⁷)Gppp(m^{2-O}) ApG, (m⁷)Gppp(m^{2′—O}) GpG, (m₂^{7,3′-O}) Gppp(m₂^{6,2′-O}) ApG, and (m⁷)Gppp(m^{2′-O}) ApU.

In some embodiments, the 5′ cap is (m₂^{7,3-O})Gppp(m^{2′-O}) ApG (“CleanCap AG”, “CC413”), having a structure:

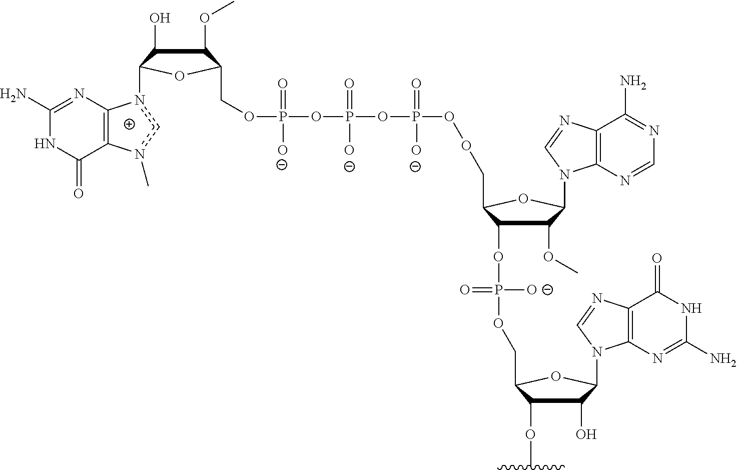

or a salt thereof.

In some embodiments, the 5′ cap is (m₂⁷⁵° C.) Gpp (m²°) GpG (“CleanCap GG”), having a structure:

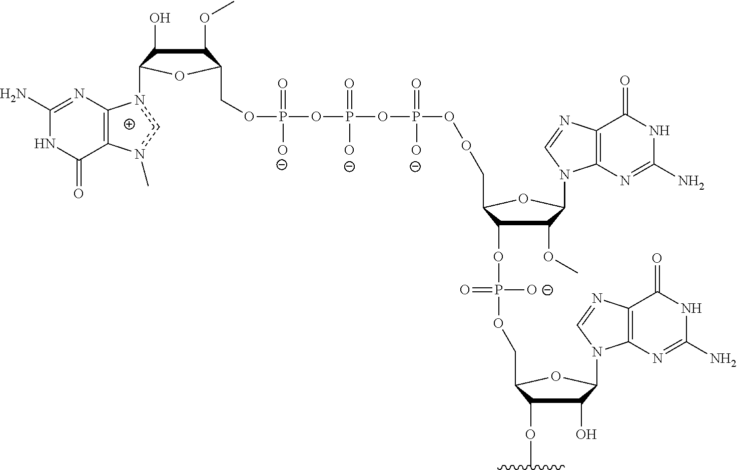

or a salt thereof.

In some embodiments, the 5′ cap is (m⁷) Gpp (m²°) ApG, having a structure:

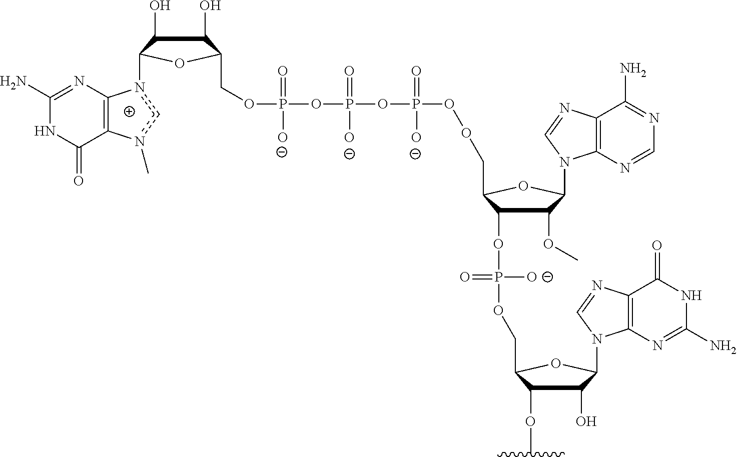

or a salt thereof.

In some embodiments, the 5′ cap is (m⁷)Gppp(m³°) GpG, having a structure:

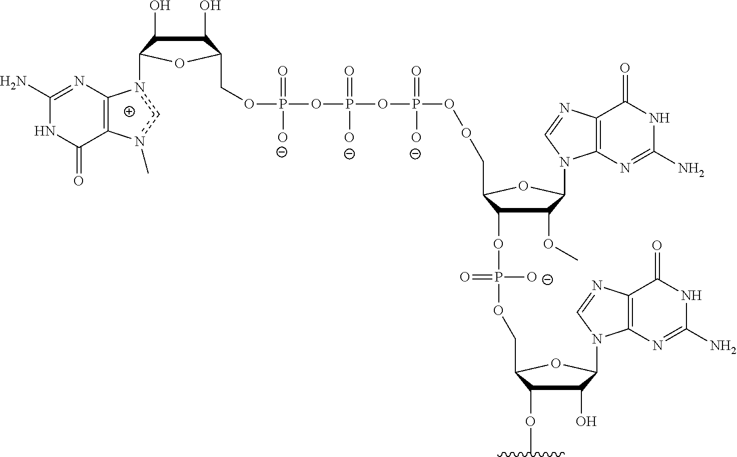

or a salt thereof.

In some embodiments, the 5′ cap is (m₂^{7,3-O}) Gpp (m²⁶-2-0) ApG, having a structure:

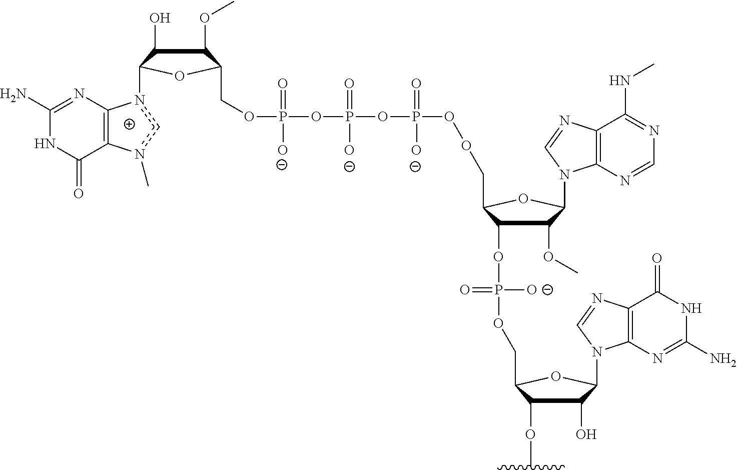

or a salt thereof.

In some embodiments, the 5′ cap is (m⁷)Gppp(m^{2-O}) ApU, having a structure:

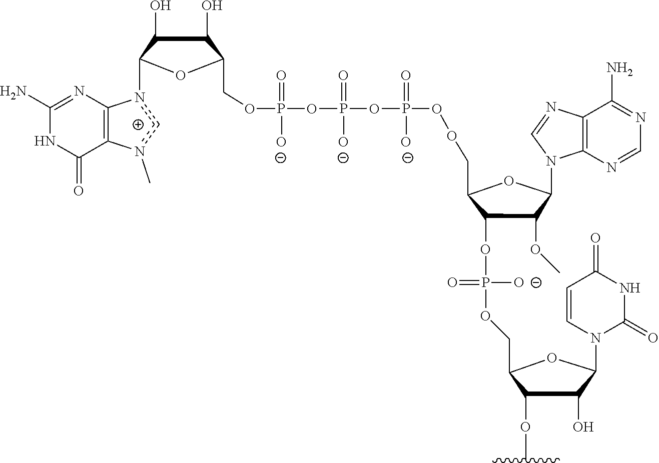

or a salt thereof.

In some embodiments, the 5′ cap is a tetranucleotide cap structure. In some embodiments, the 5′ cap is a tetranucleotide cap structure comprising N₁pN₂ PN₃, wherein N₁, N₂, and N₃ are as defined and described herein. In some embodiments, the 5′ cap is a tetranucleotide cap G*N₁pN₂PN₃, wherein N₁, N₂, and N₃ are as defined above and herein, and G* comprises a structure of formula (I):

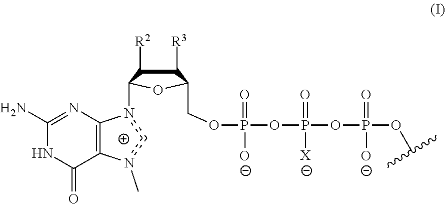

or a salt thereof, wherein R², R³, and X are as defined and described herein.

In some embodiments, the 5′ cap is a tetranucleotide cap0 structure (e.g. (m⁷)GpppN₁pN₂PN₃, (m₂^{7,2-O}) GppN₁pN₂PN₃, or (m₂^{7,3-O}) GppN₁N₂PN₃), wherein N₁, N₂, and N₃ are as defined and described herein). In some embodiments, the 5′ cap is a tetranucleotide Cap1 structure (e.g., (m⁷) Gpp (m^{2-O}) N₁PN₂PN₃, (m₂^{7,2′—O}) Gpp (m^{2-O}) N₁pN₂PN₃, (m₂^{7,3-O}) Gpp (m^{2-O}) N₁pN₂N₃), wherein N₁, N₂, and N₃ are as defined and described herein. In some embodiments, the 5′ cap is a tetranucleotide Cap2 structure (e.g., (m⁷)Gppp(m^{2-O}) NIP (m²-0) N₂PN₃, (m₂^{7,2-O}) Gpp (m^{2-O}) N₁p (m^{2-O}) N₂PN₃, (m₂⁷³-0) Gpp (m^{2-O}) N₁p (m^{2-O}) N₂PN₃), wherein N₁, N₂, and N₃ are as defined and described herein. In some embodiments, the 5′ cap is selected from the group consisting of (m₂^{7,3-O})Gppp(m^{2-O}) Ap (m^{2-O}) GpG, (m₂^{7,3-O})Gppp(m^{2-O}) Gp (m^{2-O}) GpC, (m⁷)Gppp(m^{2-O}) Ap (m^{2-O}) UpA, and (m⁷)Gppp(m^{2-O}) Ap (m^{2-O}) GpG.

In some embodiments, the 5′ cap is (m₂^{7,3-O})Gppp(m^{2′—O}) Ap (m^{2-O}) GpG, having a structure:

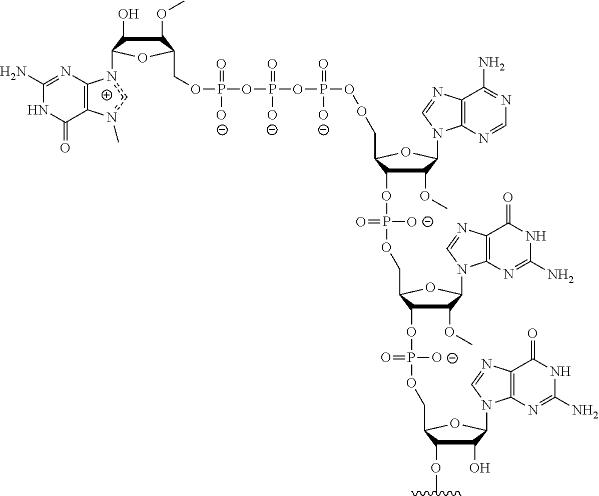

or a salt thereof.

In some embodiments, the 5′ cap is (m₂^{7,3-O})Gppp(m^{2-O}) Gp (m^{2-O}) GpC, having a structure:

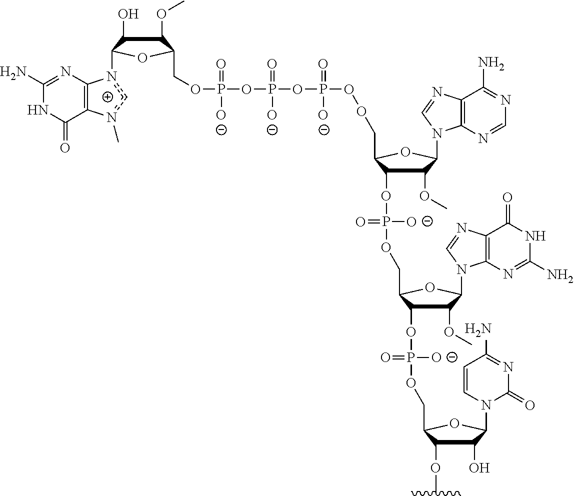

or a salt thereof.

In some embodiments, the 5′ cap is (m⁷)Gppp(m^{2-O}) Ap (m^{2-O}) UpA, having a structure:

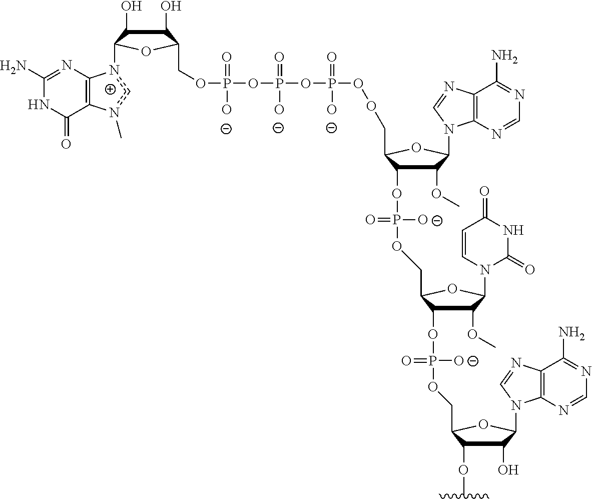

or a salt thereof.

In some embodiments, the 5′ cap is (m⁷)Gppp(m^{2-O}) Ap (m^{2-O}) GpG, having a structure:

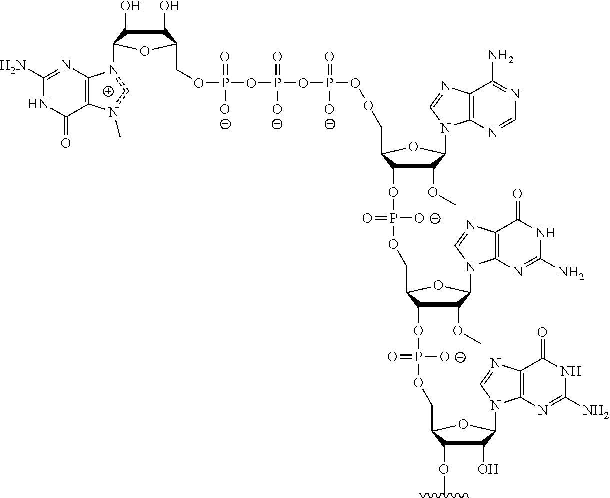

or a salt thereof.

### 2. Cap Proximal Sequences

In some embodiments, a 5′ UTR utilized in accordance with the present disclosure comprises a cap proximal sequence, e.g., as disclosed herein. In some embodiments, a cap proximal sequence comprises a sequence adjacent to a 5′ cap. In some embodiments, a cap proximal sequence comprises nucleotides in positions +1, +2, +3, +4, and/or +5 of an RNA polynucleotide.

In some embodiments, a cap structure comprises one or more polynucleotides of a cap proximal sequence. In some embodiments, a cap structure comprises an m⁷ Guanosine cap and nucleotide+1 (N₁) of an RNA polynucleotide. In some embodiments, a cap structure comprises an m⁷ Guanosine cap and nucleotide+2 (N₂) of an RNA polynucleotide. In some embodiments, a cap structure comprises an m⁷ Guanosine cap and nucleotides+1 and +2 (N₁ and N₂) of an RNA polynucleotide. In some embodiments, a cap structure comprises an m⁷ Guanosine cap and nucleotides+1, +2, and +3 (N₁, N₂, and N₃) of an RNA polynucleotide.

Those skilled in the art, reading the present disclosure, will appreciate that, in some embodiments, one or more residues of a cap proximal sequence (e.g., one or more of residues+1, +2, +3, +4, and/or +5) may be included in an RNA by virtue of having been included in a cap entity (e.g., a cap1 or cap2 structure, etc.); alternatively, in some embodiments, at least some of the residues in a cap proximal sequence may be enzymatically added (e.g., by a polymerase such as a 17 polymerase). For example, in certain exemplified embodiments where a m₂^{7,3}′—OGppp(m^{12′—O}) ApG cap is utilized, +1 (i.e., N₁) and +2 (i.e. N₂) are the (m¹²-0) A and G residues of the cap, and +3, +4, and +5 are added by polymerase (e.g., T7 polymerase).

In some embodiments, the 5′ cap is a dinucleotide cap structure, wherein the cap proximal sequence comprises N₁ of the 5′ cap, where N₁ is any nucleotide, e.g., A, C, G or U. In some embodiments, the 5′ cap is a trinucleotide cap structure (e.g., the trinucleotide cap structures described above and herein), wherein the cap proximal sequence comprises N₁ and N₂ of the 5′ cap, wherein N₁ and N₂ are independently any nucleotide, e.g., A, C, G or U. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁, N₂, and N₃ are any nucleotide, e.g., A, C, G or U.

In some embodiments, e.g., where the 5′ cap is a dinucleotide cap structure, a cap proximal sequence comprises N₁ of a the 5′ cap, and N₂, N₃, N₄ and N₅, wherein N₁ to N₅ correspond to positions +1, +2, +3, +4, and/or +5 of an RNA polynucleotide. In some embodiments, e.g., where the 5′ cap is a trinucleotide cap structure, a cap proximal sequence comprises N₁ and N₂ of a the 5′ cap, and N₃, N₄ and N₅, wherein N₁ to N₅ correspond to positions +1, +2, +3, +4, and/or +5 of an RNA polynucleotide. In some embodiments, e.g., where the 5′ cap is a tetranucleotide cap structure, a cap proximal sequence comprises N₁, N₂, and N₃ of a the 5′ cap, and N₄ and N₅, wherein N₁ to N₅ correspond to positions +1, +2, +3, +4, and/or +5 of an RNA polynucleotide.

In some embodiments, N₁ is A. In some embodiments, N₁ is C. In some embodiments, N₁ is G. In some embodiments, N₁ is U. In some embodiments, N₂ is A. In some embodiments, N₂ is C. In some embodiments, N₂ is G. In some embodiments, N₂ is U. In some embodiments, N₃ is A. In some embodiments, N₃ is C. In some embodiments, N₃ is G. In some embodiments, N₃ is U. In some embodiments, N₄ is A. In some embodiments, N₄ is C. In some embodiments, N₄ is G. In some embodiments, N₄ is U. In some embodiments, N₅ is A. In some embodiments, N₅ is C. In some embodiments, N₅ is G. In some embodiments, N₅ is U. It will be understood that, each of the embodiments described above and herein (e.g., for N₁ through N₅) may be taken singly or in combination and/or may be combined with other embodiments of variables described above and herein (e.g., 5′ caps).

### 3. 5′ UTR

In some embodiments, a nucleic acid (e.g., DNA, RNA) utilized in accordance with the present disclosure comprises a 5′-UTR. In some embodiments, 5′-UTR may comprise a plurality of distinct sequence elements; in some embodiments, such plurality may be or comprise multiple copies of one or more particular sequence elements (e.g., as may be from a particular source or otherwise known as a functional or characteristic sequence element). In some embodiments a 5′ UTR comprises multiple different sequence elements.

The term “untranslated region” or “UTR” is commonly used in the art to a region in a DNA molecule which is transcribed but is not translated into an amino acid sequence, or to the corresponding region in an RNA polynucleotide, such as an mRNA molecule. An untranslated region (UTR) can be present 5′ (upstream) of an open reading frame (5′-UTR) and/or 3′ (downstream) of an open reading frame (3′-UTR). As used herein, the terms “five prime untranslated region” or “5′ UTR” refer to a sequence of a polyribonucleotide between the 5′ end of the polyribonucleotide (e.g., a transcription start site) and a start codon of a coding region of the polyribonucleotide. In some embodiments, “5′ UTR” refers to a sequence of a polyribonucleotide that begins at the 5′ end of the polyribonucleotide (e.g., a transcription start site) and ends one nucleotide (nt) before a start codon (usually AUG) of a coding region of the polyribonucleotide, e.g., in its natural context. In some embodiments, a 5′ UTR comprises a Kozak sequence. A 5′-UTR is downstream of the 5′-cap (if present), e.g., directly adjacent to the 5′-cap. In some embodiments, a 5′ UTR disclosed herein comprises a cap proximal sequence, e.g., as defined and described herein. In some embodiments, a cap proximal sequence comprises a sequence adjacent to a 5′ cap.

Exemplary 5′ UTRs include a human alpha globin (hAg) 5′UTR or a fragment thereof, a TEV 5′ UTR or a fragment thereof, a HSP70 5′ UTR or a fragment thereof, or a c-June 5′ UTR or a fragment thereof.

In some embodiments, an RNA disclosed herein comprises a hAg 5′ UTR or a fragment thereof.

In some embodiments, an RNA disclosed herein comprises a 5′ UTR having at least 80%, at least 85%, at least 90%, at least 95%, at least 96%, at least 97%, at least 98%, or at least 99% identity to a 5′ UTR with a nucleic acid sequence according to SEQ ID NO: 415. In some embodiments, an RNA disclosed herein comprises a 5′ UTR having a nucleic acid sequence according to SEQ ID NO: 415.

### 4. PolyA Tail

In some embodiments, a polynucleotide (e.g., DNA, RNA) disclosed herein comprises a polyadenylate (polyA) sequence, e.g., as described herein. In some embodiments, a polyA sequence is situated downstream of a 3′-UTR, e.g., adjacent to a 3′-UTR.

As used herein, the term “poly(A) sequence” or “poly-A tail” refers to an uninterrupted or interrupted sequence of adenylate residues which is typically located at the 3′-end of an RNA polynucleotide. Poly(A) sequences are known to those of skill in the art and may follow the 3′-UTR in the RNAs described herein. An uninterrupted poly(A) sequence is characterized by consecutive adenylate residues. In nature, an uninterrupted poly(A) sequence is typical. In some embodiments, polynucleotides disclosed herein comprise an uninterrupted Poly(A) sequence. In some embodiments, polynucleotides disclosed herein comprise interrupted Poly(A) sequence. In some embodiments, RNAs disclosed herein can have a poly(A) sequence attached to the free 3′-end of the RNA by a template-independent RNA polymerase after transcription or a poly(A) sequence encoded by DNA and transcribed by a template-dependent RNA polymerase.

It has been demonstrated that a poly(A) sequence of about 120 A nucleotides has a beneficial influence on the levels of RNA in transfected eukaryotic cells, as well as on the levels of protein that is translated from an open reading frame that is present upstream (5′) of the poly(A) sequence (Holtkamp et al., 2006, Blood, vol. 108, pp. 4009-4017, which is herein incorporated by reference).

In some embodiments, a poly(A) sequence in accordance with the present disclosure is not limited to a particular length; in some embodiments, a poly(A) sequence is any length. In some embodiments, a poly(A) sequence comprises, essentially consists of, or consists of at least 20, at least 30, at least 40, at least 80, or at least 100 and up to 500, up to 400, up to 300, up to 200, or up to 150 A nucleotides, and, in particular, about 120 A nucleotides. In this context, “essentially consists of” means that most nucleotides in the poly(A) sequence, typically at least 75%, at least 80%, at least 85%, at least 90%, at least 95%, at least 96%, at least 97%, at least 98%, or at least 99% by number of nucleotides in the poly(A) sequence are A nucleotides, but permits that remaining nucleotides are nucleotides other than A nucleotides, such as U nucleotides (uridylate), G nucleotides (guanylate), or C nucleotides (cytidylate). In this context, “consists of” means that all nucleotides in the poly(A) sequence, i.e., 100% by number of nucleotides in the poly(A) sequence, are A nucleotides. The term “A nucleotide” or “A” refers to adenylate.

In some embodiments, a poly(A) sequence is attached during RNA transcription, e.g., during preparation of in vitro transcribed RNA, based on a DNA template comprising repeated dT nucleotides (deoxythymidylate) in the strand complementary to the coding strand. The DNA sequence encoding a poly(A) sequence (coding strand) is referred to as poly(A) cassette.

In some embodiments, the poly(A) cassette present in the coding strand of DNA essentially consists of dA nucleotides, but is interrupted by a random sequence of the four nucleotides (dA, dC, dG, and dT). Such random sequence may be 5 to 50, 10 to 30, or 10 to 20 nucleotides in length. Such a cassette is disclosed in WO 2016/005324 A1, hereby incorporated by reference. Any poly(A) cassette disclosed in WO 2016/005324 A1, which is incorporated herein by reference in its entirety, may be used in accordance with the present disclosure. A poly(A) cassette that essentially consists of dA nucleotides, but is interrupted by a random sequence having an equal distribution of the four nucleotides (dA, dC, dG, dT) and having a length of e.g., 5 to 50 nucleotides shows, on DNA level, constant propagation of plasmid DNA in E. coli and is still associated, on RNA level, with the beneficial properties with respect to supporting RNA stability and translational efficiency is encompassed. In some embodiments, the poly(A) sequence contained in an RNA polynucleotide described herein essentially consists of A nucleotides, but is interrupted by a random sequence of the four nucleotides (A, C, G, U). Such random sequence may be 5 to 50, 10 to 30, or 10 to 20 nucleotides in length.

In some embodiments, no nucleotides other than A nucleotides flank a poly(A) sequence at its 3′-end, i.e., the poly(A) sequence is not masked or followed at its 3′-end by a nucleotide other than A.

In some embodiments, the poly(A) sequence may comprise at least 20, at least 30, at least 40, at least 80, or at least 100 and up to 500, up to 400, up to 300, up to 200, or up to 150 nucleotides. In some embodiments, the poly(A) sequence may essentially consist of at least 20, at least 30, at least 40, at least 80, or at least 100 and up to 500, up to 400, up to 300, up to 200, or up to 150 nucleotides. In some embodiments, the poly(A) sequence may consist of at least 20, at least 30, at least 40, at least 80, or at least 100 and up to 500, up to 400, up to 300, up to 200, or up to 150 nucleotides. In some embodiments, the poly(A) sequence comprises at least 100 nucleotides. In some embodiments, the poly(A) sequence comprises about 150 nucleotides. In some embodiments, the poly(A) sequence comprises about 120 nucleotides.

In some embodiments, a poly A tail comprises a specific number of Adenosines, such as about 50 or more, about 60 or more, about 70 or more, about 80 or more, about 90 or more, about 100 or more, about 120, or about 150 or about 200. In some embodiments a poly A tail of a string construct may comprise 200 A residues or less. In some embodiments, a poly A tail of a string construct may comprise about 200 A residues. In some embodiments, a poly A tail of a string construct may comprise 180 A residues or less. In some embodiments, a poly A tail of a string construct may comprise about 180 A residues. In some embodiments, a poly A tail may comprise 150 residues or less.

In some embodiments, RNA comprises a poly(A) sequence comprising a nucleotide sequence of according to SEQ ID NO: 428, or a nucleotide sequence having at least 99%, 98%, 97%, 96%, 95%, 90%, 85%, or 80% identity to a nucleotide sequence of according to SEQ ID NO: 428. In some embodiments, a poly(A) tail comprises a plurality of A residues interrupted by a linker. In some embodiments, a linker comprises the nucleotide sequence GCATATGAC.

### 5. 3′ UTR

In some embodiments, an RNA utilized in accordance with the present disclosure comprises a 3′-UTR. As used herein, the terms “three prime untranslated region,” “3′ untranslated region,” or “3′ UTR” refer to a sequence of an mRNA molecule that begins following a stop codon of a coding region of an open reading frame sequence. In some embodiments, the 3′ UTR begins immediately after a stop codon of a coding region of an open reading frame sequence, e.g., in its natural context. In other embodiments, the 3′ UTR does not begin immediately after stop codon of the coding region of an open reading frame sequence, e.g., in its natural context. The term “3′-UTR” does preferably not include the poly(A) sequence. Thus, the 3′-UTR is upstream of the poly(A) sequence (if present), e.g. directly adjacent to the poly(A) sequence.

In some embodiments, an RNA disclosed herein comprises a 3′ UTR comprising an F element and/or an I element. In some embodiments, a 3′ UTR or a proximal sequence thereto comprises a restriction site. In some embodiments, a restriction site is a BamHI site. In some embodiments, a restriction site is a XhoI site.

In some embodiments, an RNA construct comprises an F element. In some embodiments, a F element sequence is a 3′-UTR of amino-terminal enhancer of split (AES).

In some embodiments, an RNA disclosed herein comprises a 3′ UTR having at least 80%, at least 85%, at least 90%, at least 95%, at least 96%, at least 97%, at least 98%, or at least 99% identity to a 3′ UTR with a nucleic acid sequence according to SEQ ID NO: 416. In some embodiments, an RNA disclosed herein comprises a 3′ UTR with a nucleic acid sequence according to SEQ ID NO: 416.

In some embodiments, a 3′UTR is an FI element as described in WO2017/060314, which is herein incorporated by reference in its entirety.

### B. RNA Formats

At least three distinct formats useful for RNA compositions (e.g., pharmaceutical compositions) have been developed, namely non-modified uridine containing mRNA (uRNA), nucleoside-modified mRNA (modRNA), and self-amplifying mRNA (saRNA). Each of these platforms displays unique features. In general, in all three formats, RNA is capped, contains open reading frames (ORFs) flanked by untranslated regions (UTR), and have a polyA-tail at the 3′ end. An ORF of an uRNA and modRNA vectors encode an antibody agent or portion thereof. An saRNA has multiple ORFs.

In some embodiments, the RNA described herein may have modified nucleosides. In some embodiments, the RNA comprises a modified nucleoside in place of at least one (e.g., every) uridine.

The term “uracil,” as used herein, describes one of the nucleobases that can occur in the nucleic acid of RNA. The structure of uracil is:

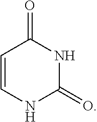

The term “uridine,” as used herein, describes one of the nucleosides that can occur in RNA. The structure of uridine is:

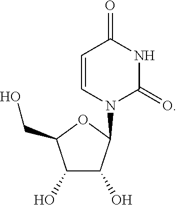 [SMILES: O=c1ccn([C@@H]2O[C@H](CO)[C@@H](O)[C@H]2O)c(=O)[nH]1]

UTP (uridine 5′-triphosphate) has the following structure:

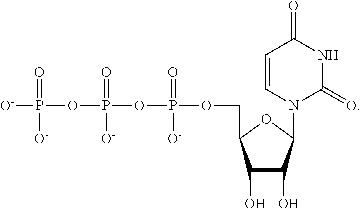

Pseudo-UTP (pseudouridine 5′-triphosphate) has the following structure:

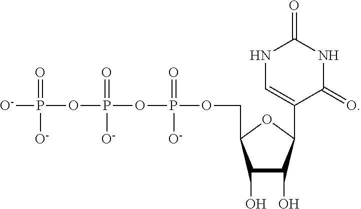

“Pseudouridine” is one example of a modified nucleoside that is an isomer of uridine, where the uracil is attached to the pentose ring via a carbon-carbon bond instead of a nitrogen-carbon glycosidic bond.

Another exemplary modified nucleoside is N₁-methyl-pseudouridine (m¹), which has the structure:

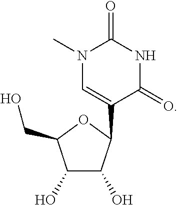 [SMILES: Cn1cc([C@@H]2O[C@H](CO)[C@@H](O)[C@H]2O)c(=O)[nH]c1=O]

N₁-methyl-pseudo-UTP has the following structure:

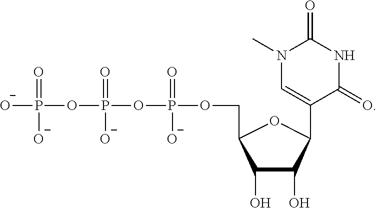

Another exemplary modified nucleoside is 5-methyl-uridine (m⁵U), which has the structure:

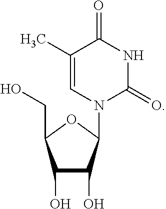

In some embodiments, one or more uridine in the RNA described herein is replaced by a modified nucleoside. In some embodiments, the modified nucleoside is a modified uridine.

In some embodiments, RNA comprises a modified nucleoside in place of at least one uridine. In some embodiments, RNA comprises a modified nucleoside in place of each uridine.

In some embodiments, the modified nucleoside is independently selected from pseudouridine (w), N₁-methyl-pseudouridine (m1ψ), and 5-methyl-uridine (m⁵U). In some embodiments, the modified nucleoside comprises pseudouridine (w). In some embodiments, the modified nucleoside comprises N₁-methyl-pseudouridine (m1ψ). In some embodiments, the modified nucleoside comprises 5-methyl-uridine (m⁵U). In some embodiments, RNA may comprise more than one type of modified nucleoside, and the modified nucleosides are independently selected from pseudouridine (w), N₁-methyl-pseudouridine (m1ψ), and 5-methyl-uridine (m⁵U). In some embodiments, the modified nucleosides comprise pseudouridine (w) and N₁-methyl-pseudouridine (m1ψ). In some embodiments, the modified nucleosides comprise pseudouridine (w) and 5-methyl-uridine (m⁵U). In some embodiments, the modified nucleosides comprise N₁-methyl-pseudouridine (m1ψ) and 5-methyl-uridine (m⁵U). In some embodiments, the modified nucleosides comprise pseudouridine (w), N₁-methyl-pseudouridine (m1ψ), and 5-methyl-uridine (m⁵U).

In some embodiments, the modified nucleoside replacing one or more, e.g., all, uridine in the RNA may be any one or more of 3-methyl-uridine (m³U), 5-methoxy-uridine (mo5U), 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridine or 5-bromo-uridine), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 1-ethyl-pseudouridine, 5-methylaminomethyl-2-thio-uridine (mnm5s2U), 5-methylaminomethyl-2-seleno-uridine (mnm5se2U), 5-carbamoylmethyl-uridine (ncm5U), 5-carboxymethylaminomethyl-uridine (cmnm5U), 5-carboxymethylaminomethyl-2-thio-uridine (cmnm5s2U), 5-propynyl-uridine, 1-propynyl-pseudouridine, 5-taurinomethyl-uridine (Tm5U), 1-taurinomethyl-pseudouridine, 5-taurinomethyl-2-thio-uridine (m⁵s2U), 1-taurinomethyl-4-thio-pseudouridine), 5-methyl-2-thio-uridine (m⁵s2U), 1-methyl-4-thio-pseudouridine (m¹s4w), 4-thio-1-methyl-pseudouridine, 3-methyl-pseudouridine (m³w), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N₁-methyl-pseudouridine, 3-(3-amino-3-carboxypropyl) uridine (acp3U), 1-methyl-3-(3-amino-3-carboxypropyl) pseudouridine (acp3 w), 5-(isopentenylaminomethyl) uridine (inm5U), 5-(isopentenylaminomethyl)-2-thio-uridine (inm5s2U), α-thio-uridine, 2′—O-methyl-uridine (Um), 5,2′—O-dimethyl-uridine (m⁵Um), 2′—O-methyl-pseudouridine (ψm), 2-thio-2′—O-methyl-uridine (s2Um), 5-methoxycarbonylmethyl-2′—O-methyl-uridine (mcm5Um), 5-carbamoylmethyl-2′—O-methyl-uridine (ncm5Um), 5-carboxymethylaminomethyl-2′—O-methyl-uridine (cmnm5Um), 3,2′—O-dimethyl-uridine (m³Um), 5-(isopentenylaminomethyl)-2′—O-methyl-uridine (inm5Um), 1-thio-uridine, deoxythymidine, 2′-F-ara-uridine, 2′-F-uridine, 2′—OH-ara-uridine, 5-(2-carbomethoxyvinyl) uridine, 5-[3-(1-E-propenylamino) uridine, or any other modified uridine known in the art.

In some embodiments, the RNA comprises other modified nucleosides or comprises further modified nucleosides, e.g., modified cytidine. For example, in some embodiments, in the RNA 5-methylcytidine is substituted partially or completely, preferably completely, for cytidine. In some embodiments, the RNA comprises 5-methylcytidine and one or more selected from pseudouridine (w), N₁-methyl-pseudouridine (m1ψ), and 5-methyl-uridine (m⁵U). In some embodiments, the RNA comprises 5-methylcytidine and N₁-methyl-pseudouridine (m1ψ). In some embodiments, the RNA comprises 5-methylcytidine in place of each cytidine and N₁-methyl-pseudouridine (m1ψ) in place of each uridine.

In some embodiments of the present disclosure, the RNA is “replicon RNA” or simply a “replicon,” in particular “self-replicating RNA” or “self-amplifying RNA.” In one particularly preferred embodiment, the replicon or self-replicating RNA is derived from or comprises elements derived from a single-stranded (ss) RNA virus, in particular a positive-stranded ssRNA virus, such as an alphavirus. Alphaviruses are typical representatives of positive-stranded RNA viruses. Alphaviruses replicate in the cytoplasm of infected cells (for review of the alphaviral life cycle see José et al., Future Microbiol., 2009, vol. 4, pp. 837-856, which is incorporated herein by reference in its entirety). The total genome length of many alphaviruses typically ranges between 11,000 and 12,000 nucleotides, and the genomic RNA typically has a 5′-cap, and a 3′ poly(A) tail. The genome of alphaviruses encodes non-structural proteins (involved in transcription, modification and replication of viral RNA and in protein modification) and structural proteins (forming the virus particle). There are typically two open reading frames (ORFs) in the genome. The four non-structural proteins (nsP1-nsP4) are typically encoded together by a first ORF beginning near the 5′ terminus of the genome, while alphavirus structural proteins are encoded together by a second ORF which is found downstream of the first ORF and extends near the 3′ terminus of the genome. Typically, the first ORF is larger than the second ORF, the ratio being roughly 2:1. In cells infected by an alphavirus, only the nucleic acid sequence encoding non-structural proteins is translated from the genomic RNA, while the genetic information encoding structural proteins is translatable from a subgenomic transcript, which is an RNA molecule that resembles eukaryotic messenger RNA (mRNA; Gould et al., 2010, Antiviral Res., vol. 87 pp. 111-124, which is incorporated herein by reference in its entirety). Following infection, i.e. at early stages of the viral life cycle, the (+) stranded genomic RNA directly acts like a messenger RNA for the translation of the open reading frame encoding the non-structural poly-protein (nsP1234).

Alphavirus-derived vectors have been proposed for delivery of foreign genetic information into target cells or target organisms. In simple approaches, a first ORF encodes an alphavirus-derived RNA-dependent RNA polymerase (replicase), which upon translation mediates self-amplification of the RNA. A second ORF encoding alphaviral structural proteins is replaced by an open reading frame encoding a Plasmodium polypeptide construct described herein. Alphavirus-based trans-replication systems rely on alphavirus nucleotide sequence elements on two separate nucleic acid molecules: one nucleic acid molecule encodes a viral replicase, and the other nucleic acid molecule is capable of being replicated by said replicase in trans (hence the designation trans-replication system). Trans-replication requires the presence of both these nucleic acid molecules in a given host cell. The nucleic acid molecule capable of being replicated by the replicase in trans must comprise certain alphaviral sequence elements to allow recognition and RNA synthesis by the alphaviral replicase.

Features of a non-modified uridine platform may include, for example, one or more of intrinsic adjuvant effect, as well as good tolerability and safety. Features of modified uridine (e.g., pseudouridine) platform may include reduced adjuvant effect, blunted immune innate immune sensor activating capacity and thus good tolerability and safety. Features of self-amplifying platform may include, for example, long duration of protein expression, good tolerability and safety, higher likelihood for efficacy with very low vaccine dose.

The present disclosure provides particular RNA constructs optimized, for example, for improved manufacturability, encapsulation, expression level (and/or timing), etc. Certain components are discussed below, and certain preferred embodiments are exemplified herein.

### C. Codon Optimization and GC Enrichment

As used herein, the term “codon-optimized” refers to alteration of codons in a coding region of a nucleic acid molecule (e.g., a polyribonucleotide) to reflect the typical codon usage of a host organism (e.g., a subject receiving a nucleic acid molecule (e.g., a polyribonucleotide)) without preferably altering the amino acid sequence encoded by the nucleic acid molecule. Within the context of the present disclosure, in some embodiments, coding regions are codon-optimized for optimal expression in a subject to be treated using the RNA molecules described herein. In some embodiments, codon-optimization may be performed such that codons for which frequently occurring tRNAs are available are inserted in place of “rare codons.” In some embodiments, codon-optimization may include increasing guanosine/cytosine (G/C) content of a coding region of RNA described herein as compared to the G/C content of the corresponding coding sequence of a wild type RNA, wherein the amino acid sequence encoded by the RNA is preferably not modified compared to the amino acid sequence.

In some embodiments, a coding sequence (also referred to as a “coding region”) is codon optimized for expression in the subject to whom a composition (e.g., a pharmaceutical composition) is to be administered (e.g., a human). Thus, in some embodiments, sequences in such a polynucleotide (e.g., a polyribonucleotide) may differ from wild type sequences encoding the relevant antigen or fragment or epitope thereof, even when the amino acid sequence of the antigen or fragment or epitope thereof is wild type.

In some embodiments, strategies for codon optimization for expression in a relevant subject (e.g., a human), and even, in some cases, for expression in a particular cell or tissue.

Various species exhibit particular bias for certain codons of a particular amino acid. Without wishing to be bound by any one theory, codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell may generally be a reflection of the codons used most frequently in peptide synthesis. Accordingly, genes may be tailored for optimal gene expression in a given organism based on codon optimization. Codon usage tables are available, for example, at the “Codon Usage Database” available at www.kazusa.orjp/codon/and these tables may be adapted in a number of ways. Computer algorithms for codon optimizing a particular sequence for expression in a particular subject or its cells are also available, such as Gene Forge (Aptagen; Jacobus, PA), are also available.

In some embodiments, a polynucleotide (e.g., a polyribonucleotide) of the present disclosure is codon optimized, wherein the codons in the polynucleotide (e.g., the polyribonucleotide) are adapted to human codon usage (herein referred to as “human codon optimized polynucleotide”). Codons encoding the same amino acid occur at different frequencies in a subject, e.g., a human. Accordingly, in some embodiments, the coding sequence of a polynucleotide of the present disclosure is modified such that the frequency of the codons encoding the same amino acid corresponds to the naturally occurring frequency of that codon according to the human codon usage, e.g., as shown in Table 7. For example, in the case of the amino acid Ala, the wild type coding sequence is preferably adapted in a way that the codon “GCC” is used with a frequency of 0.40, the codon “GCT” is used with a frequency of 0.28, the codon “GCA” is used with a frequency of 0.22 and the codon “GCG” is used with 30 a frequency of 0.10 etc. (see Table 7). Accordingly, in some embodiments, such a procedure (as exemplified for Ala) is applied for each amino acid encoded by the coding sequence of a polynucleotide to obtain sequences adapted to human codon usage.

TABLE 7
Human codon usage table with frequencies
indicated for each amino acid

| Amino acid | codon | frequency | Amino acid | codon | frequency |
|---|---|---|---|---|---|
| Ala | GCG | 0.10 | Pro | CCG | 0.11 |
| Ala | GCA | 0.22 | Pro | CCA | 0.27 |
| Ala | GCT | 0.28 | Pro | CCT | 0.29 |
| Ala | GCC* | 0.40 | Pro | CCC* | 0.33 |
| Cys | TGT | 0.42 | Gin | CAG* | 0.73 |
| Cys | TGC* | 0.58 | Gin | CAA | 0.27 |
| Asp | GAT | 0.44 | Arg | AGG | 0.22 |
| Asp | GAC* | 0.56 | Arg | AGA* | 0.21 |
| Glu | GAG* | 0.59 | Arg | CGG | 0.19 |
| Glu | GAA | 0.41 | Arg | CGA | 0.10 |
| Phe | TTT | 0.43 | Arg | CGT | 0.09 |
| Phe | TTC* | 0.57 | Arg | CGC | 0.19 |
| Gly | GGG | 0.23 | Ser | AGT | 0.14 |
| Gly | GGA | 0.26 | Ser | AGC* | 0.25 |
| Gly | GGT | 0.18 | Ser | TCG | 0.06 |
| Gly | GGC* | 0.33 | Ser | TCA | 0.15 |
| His | CAT | 0.41 | Ser | TCT | 0.18 |
| His | CAC* | 0.59 | Ser | TCC | 0.23 |
| Ile | ATA | 0.14 | Thr | ACG | 0.12 |
| Ile | ATT | 0.35 | Thr | ACA | 0.27 |
| Ile | ATC* | 0.52 | Thr | ACT | 0.23 |
| Lys | AAG* | 0.60 | Tor | ACC* | 0.38 |
| Lys | AAA | 0.40 | Val | GTG* | 0.48 |
| Leu | TTG | 0.12 | Val | GTA | 0.10 |
| Leu | TTA | 0.06 | Val | GTT | 0.17 |
| Leu | CTG* | 0.43 | Val | GTC | 0.25 |
| Leu | CTA | 0.07 | Trp | TGG* | 1 |
| Leu | CTT | 0.12 | Tyr | TAT | 0 42 |
| Lou | CTC | 0.20 | Tyr | TAC* | 0.58 |
| Met | ATG* | 1 | Stop | TGA* | 0 61 |
| Asn | AAT | 0.44 | Stop | TAG | 0.17 |
| Asn | AAC* | 0.56 | Stop | TAA | 0.22 |

Certain strategies for codon optimization and/or G/C enrichment for human expression are described in WO2002/098443, which is incorporated by reference herein in its entirety. In some embodiments, a coding sequence may be optimized using a multiparametric optimization strategy. In some embodiments, optimization parameters may include parameters that influence protein expression, which can be, for example, impacted on a transcription level, an mRNA level, and/or a translational level. In some embodiments, exemplary optimization parameters include, but are not limited to transcription-level parameters (including, e.g., GC content, consensus splice sites, cryptic splice sites, SD sequences, TATA boxes, termination signals, artificial recombination sites, and combinations thereof); mRNA-level parameters (including, e.g., RNA instability motifs, ribosomal entry sites, repetitive sequences, and combinations thereof); translation-level parameters (including, e.g., codon usage, premature poly(A) sites, ribosomal entry sites, secondary structures, and combinations thereof); or combinations thereof. In some embodiments, a coding sequence may be optimized by a GeneOptimizer algorithm as described in Fath et al. “Multiparameter RNA and Codon Optimization: A Standardized Tool to Assess and Enhance Autologous Mammalian Gene Expression” PLoS ONE 6 (3): e17596; Rabb et al., which is incorporated herein by reference in its entirety, “The GeneOptimizer Algorithm: using a sliding window approach to cope with the vast sequence space in multiparameter DNA sequence optimization” Systems and Synthetic Biology (2010) 4:215-225; and Graft et al. “Codon-optimized genes that enable increased heterologous expression in mammalian cells and elicit efficient immune responses in mice after vaccination of naked DNA” Methods Mol Med (2004) 94:197-210, the entire content of each of which is incorporated herein for the purposes described herein. In some embodiments, a coding sequence may be optimized by Eurofins' adaption and optimization algorithm “GENEius” as described in Eurofins' Application Notes: Eurofins' adaption and optimization software “GENEius” in comparison to other optimization algorithms, the entire content of which is incorporated by reference for the purposes described herein.

In some embodiments, a coding sequence utilized in accordance with the present disclosure has G/C content that is increased compared to a wild type coding sequence for a malarial construct described herein, or a portion thereof. In some embodiments, guanosine/cytidine (G/C) content of a coding region is modified relative to a wild type coding sequence for a malarial construct described herein, but the amino acid sequence encoded by the polyribonucleotide not modified.

Without wishing to be bound by any particular theory, it is proposed that GC enrichment may improve translation of a payload sequence. Typically, sequences having an increased G (guanosine)/C (cytidine) content are more stable than sequences having an increased A (adenosine)/U (uridine) content. In respect to the fact that several codons code for one and the same amino acid (so-called degeneration of the genetic code), the most favorable codons for the stability can be determined (so-called alternative codon usage). Depending on the amino acid to be encoded by a polyribonucleotide, there are various possibilities for modification of the ribonucleic acid sequence, compared to its wild type sequence. In particular, codons which contain A and/or U nucleosides can be modified by substituting these codons by other codons, which code for the same amino acids but contain no A and/or U or contain a lower content of A and/or U nucleosides.

In some embodiments, G/C content of a coding region of a polyribonucleotide described herein is increased by at least 1%, at least 2%, at least 3%, at least 4%, at least 5%, at least 6%, or even more compared to the G/C content of the coding region prior to codon optimization, e.g., of the wild type RNA. In some embodiments, G/C content of a coding region of a polyribonucleotide described herein is decreased by at least 1%, at least 2%, at least 3%, at least 4%, at least 5%, at least 6%, or even more compared to the G/C content of the coding region prior to codon optimization, e.g., of the wild type RNA.

In some embodiments, stability and translation efficiency of a polyribonucleotide may incorporate one or more elements established to contribute to stability and/or translation efficiency of the polyribonucleotide; exemplary such elements are described, for example, in PCT/EP2006/009448 incorporated herein by reference. In some embodiments, to increase expression of a polyribonucleotide used according to the present disclosure, a polyribonucleotide may be modified within the coding region, i.e., the sequence encoding the expressed peptide or protein, without altering the sequence of the expressed peptide or protein, for example so as to increase the GC-content to increase mRNA stability and/or to perform a codon optimization and, thus, enhance translation in cells.

### D. Exemplary Configuration

Among other things, the present disclosure provides polyribonucleotides encoding a Plasmodium polypeptide construct that comprises a viral secretory signal with one or more Plasmodium CSP polypeptide regions or portions thereof. The present disclosure provides the insight that such a polyribonucleotide will result in a polypeptide that can be effectively presented to a subject's immune system and induce an immune response. Accordingly, the present disclosure provides the recognition that such polypeptides, and encoding polyribonucleotides, are particularly effective in pharmaceutical compositions (e.g., immunogenic compositions, e.g., vaccines) that will be administered a subject (e.g., a human).

In some embodiments, the present disclosure provides a polyribonucleotide encoding a Plasmodium polypeptide construct that comprises an HSV-1 gD secretory signal and/or an HSV-1 gD transmembrane domain with one or more parasitic antigens. In some embodiments, the present disclosure provides a polyribonucleotide encoding a Plasmodium polypeptide construct that comprises an HSV-1 gD secretory signal and/or an HSV-1 gD transmembrane domain with one or more malarial antigens. In some embodiments, the present disclosure provides a polyribonucleotide encoding a Plasmodium polypeptide construct that comprises an HSV-1 gD secretory signal and/or an HSV-1 gD transmembrane domain with one or more Plasmodium CSP polypeptide regions or portions thereof.

In some embodiments, a polyribonucleotide can encode a polypeptide, where the polypeptide comprises an HSV-1 gD secretory signal, one or more Plasmodium CSP polypeptide regions or portions thereof, and an HSV-1 gD transmembrane domain. In some embodiments, a method can comprise administering a polyribonucleotide encoding a polypeptide to a subject, where the polypeptide comprises an HSV-1 gD secretory signal, one or more Plasmodium CSP polypeptide regions or portions thereof, and an HSV-1 gD transmembrane domain, and where the subject is a mammal (e.g., a human).

In some embodiments, an HSV-1 gD secretory signal comprises or consists of an amino acid sequence having at least 95% identity to the amino acid sequence of SEQ ID NO: 159. In some embodiments, an HSV-1 gD secretory signal comprises or consists of the amino acid sequence of SEQ ID NO: 159. In some embodiments, an HSV-1 gD secretory signal comprises or consists of an amino acid sequence having at least 95% identity to the amino acid sequence of SEQ ID NO: 165. In some embodiments, an HSV-1 gD secretory signal comprises or consists of the amino acid sequence of SEQ ID NO: 165.

In some embodiments, an HSV-1 gD transmembrane domain comprises or consists of an amino acid sequence having at least 95% identity to the amino acid sequence of SEQ ID NO:234. In some embodiments, an HSV-1 gD transmembrane domain comprises or consists of the amino acid sequence of SEQ ID NO: 234.

In some embodiments, one or more Plasmodium CSP polypeptide regions or portions thereof are as described herein.

### E. Embodiments of Polyribonucleotides Encoding Plasmodium Polypeptide Constructs

In the following, exemplary embodiments of polyribonucleotides encoding Plasmodium polypeptide constructs are described, wherein certain terms used when describing elements thereof have the following meanings:
cap: 5′-cap structure, e.g., selected from the group consisting of m27,2′-OG (5′)ppSp(5′)G (in particular its D1 diastereomer), m₂7,3′-OG (5′) ppp (5′) G, and m₂7,3′—OGppp(m12′—O) ApG.
hAg-Kozak: 5′-UTR sequence of the human alpha-globin mRNA with an optimized ‘Kozak sequence’ to increase translational efficiency.
sec: Sequences encoding a secretory signal.
Antigen: Sequences encoding one or more Plasmodium polypeptide constructs or portions or variants thereof (e.g., immunogenic fragments of the Plasmodium polypeptide constructs or the immunogenic variants thereof), from Plasmodium falciparum, preferably Plasmodium falciparum isolate 3D7.
TMD: Sequences encoding a transmembrane region.
Linker: Sequences coding for peptide linkers.
FI element: The 3′-UTR is a combination of two sequence elements derived from the “amino terminal enhancer of split” (AES) mRNA (called F) and the mitochondrial encoded 12S ribosomal RNA (called I). These were identified by an ex vivo selection process for sequences that confer RNA stability and augment total protein expression.
A30L70: A poly(A)-tail measuring 110 nucleotides in length, consisting of a stretch of 30 adenosine residues, followed by a 10 nucleotide linker sequence and another 70 adenosine residues designed to enhance RNA stability and translational efficiency in dendritic cells.

In some embodiments, a polyribonucleotide encoding a Plasmodium polypeptide construct described herein has one of the following structures:
cap-hAg-Kozak-Antigen-FI-A30L70;
cap-hAg-Kozak-sec-Antigen-FI-A30L70;
cap-hAg-Kozak-Antigen-TMD-FI-A30L70; or
cap-hAg-Kozak-sec-Antigen-TMD-FI-A30L70.

In some embodiments, hAg-Kozak comprises the nucleotide sequence of SEQ ID NO: 415. In some embodiments, FI comprises the nucleotide sequence of SEQ ID NO: 416. In some embodiments, A30L70 comprises the nucleotide sequence of SEQ ID NO: 417.

In some embodiments, the secretory signal is from Plasmodium falciparum, preferably Plasmodium falciparum isolate 3D7, and referred to herein as Pfsec and has the sequence as defined in SEQ ID NO: 174. In some embodiments, the secretory signal is heterologous and selected from the amino acid sequences as provided in Table 3. In some embodiments, the secretory signal is HSV1-gD and referred to herein as HSV-1gDsec and has the sequence as defined in SEQ ID NO: 159, SEQ ID NO: 162, or SEQ ID NO: 165.

In some embodiments, the TMD is a glycosylphosphatidylinositol (GPI) anchor region from Plasmodium CSP, preferably from Plasmodium falciparum isolate 3D7, and referred to herein as PfTMD and has the amino acid sequence of SEQ ID NO. 231. In some embodiments, the TMD is heterologous. In some embodiments, the TMD is from HSV1-gD and referred to herein as HSV-1TMD and has the amino acid sequence of SEQ ID NO:234.

In some embodiments, the Antigen comprises a full-length CSP construct as defined above, a N-terminal region deletion CSP construct as defined above, a N-terminal region and major region deleted CSP construct as defined above, a N-terminal domain deleted CSP construct as defined above, a N-terminal domain and major repeat region deleted CSP construct as defined above, a N-terminal domain and major repeat region deleted CSP construct with modified junction region variants or portions as defined above or a major repeat region portion and C-terminal region containing CSP constructs as defined above. In some embodiments, a polyribonucleotide described herein has one of the following structures:
cap-hAg.Kozak-full-length CSP construct-FI-A30L70;
cap-hAg-Kozak-sec-full-length CSP construct-FI-A30L70;
cap-hAg-Kozak-full-length CSP construct-TMD-FI-A30L70;
cap-hAg-Kozak-sec-full-length CSP construct-TMD-FI-A30L70;
cap-hAg-Kozak-Pfsec-full-length CSP construct-FI-A30L70;
cap-hAg-Kozak-full-length CSP construct-PfTMD-FI-A30L70;
cap-hAg-Kozak-Pfsec-full-length CSP construct-PfTMD-FI-A30L70;
cap-hAg-Kozak-HSV-1gDsec-full-length CSP construct-FI-A30L70;
cap-hAg-Kozak-full-length CSP construct-HSV-1TMD-FI-A30L70;
cap-hAg-Kozak-HSV-1gDsec-full-length CSP construct-HSV-1TMD-FI-A30L70;
cap-hAg-Kozak-Pfsec-full-length CSP construct-HSV-1TMD-FI-A30L70;
cap-hAg-Kozak-HSV-1gDsec-full-length CSP construct-PTMD-FI-A30L70;
cap-hAg-Kozak-heterologoussec-full-length CSP construct-FI-A30L70;
cap-hAg-Kozak-full-length CSP construct-heterologousTMD-FI-A30L70;
cap-hAg-Kozak-heterologoussec-full-length CSP construct-heterologousTMD-FI-A30L70;
cap-hAg-Kozak-dNT CSP construct-FI-A30L70;
cap-hAg-Kozak-sec-dNT CSP construct-FI-A30L70;
cap-hAg-Kozak-dNT CSP construct-TMD-FI-A30L70;
cap-hAg-Kozak-sec-dNT CSP construct-TMD-FI-A30L70;
cap-hAg-Kozak-Pfsec-dNT CSP construct-FI-A30L70;
cap-hAg-Kozak-dNT CSP construct-PfTMD-FI-A30L70;
cap-hAg-Kozak-Pfsec-dNT CSP construct-PfTMD-FI-A30L70;
cap-hAg-Kozak-HSV-1gDsec-dNT CSP construct-FI-A30L70;
cap-hAg-Kozak-dNT CSP construct-HSV-1TMD-FI-A30L70;
cap-hAg-Kozak-HSV-1gDsec-dNT CSP construct-HSV-1TMD-FI-A30L70;
cap-hAg-Kozak-HSV-1gDsec-dNT CSP construct-PfTMD-FI-A30L70;
cap-hAg-Kozak-Pfsec-dNT CSP construct-HSV-1TMD-FI-A30L70;
cap-hAg-Kozak-heterologoussec-dNT CSP construct-FI-A30L70;
cap-hAg-Kozak-dNT CSP construct-heterologousTMD-FI-A30L70;
cap-hAg-Kozak-heterologoussec-dNT CSP construct-heterologousTMD-FI-A30L70;
cap-hAg-Kozak-dNT-dmajor CSP construct-FI-A30L70;
cap-hAg-Kozak-sec-dNT-dmajor CSP construct-FI-A30L70;
cap-hAg-Kozak-dNT-dmajor CSP construct-TMD-FI-A30L70;
cap-hAg-Kozak-sec-dNT-dmajor CSP construct-TMD-FI-A30L70;
cap-hAg-Kozak-Pfsec-dNT-dmajor CSP construct-FI-A30L70;
cap-hAg-Kozak-dNT-dmajor CSP construct-PTMD-FI-A30L70;
cap-hAg-Kozak-Pfsec-dNT-dmajor CSP construct-PfTMD-FI-A30L70;
cap-hAg-Kozak-HSV-1gDsec-dNT-dmajor CSP construct-FI-A30L70;
cap-hAg-Kozak-dNT-dmajor CSP construct-HSV-1TMD-FI-A30L70;
cap-hAg-Kozak-HSV-1gDsec-dNT-dmajor CSP construct-HSV-1TMD-FI-A30L70;
cap-hAg-Kozak-HSV-1gDsec-dNT-dmajor CSP construct-PfTMD-FI-A30L70;
cap-hAg-Kozak-Pfsec-dNT-dmajor CSP construct-HSV-1TMD-FI-A30L70;
cap-hAg-Kozak-heterologoussec-dNT-dmajor CSP construct-FI-A30L70;
cap-hAg-Kozak-dNT-dmajor CSP construct-heterologousTMD-FI-A30L70;
cap-hAg-Kozak-heterologoussec-dNT-dmajor CSP construct-heterologousTMD-FI-A30L70;
cap-hAg-Kozak-dNT-dmajor CSP construct-helper antigen-FI-A30L70;
cap-hAg-Kozak-sec-dNT-dmajor CSP construct-helper antigen-FI-A30L70;
cap-hAg-Kozak-dNT-dmajor CSP construct-TMD-helper antigen-FI-A30L70;
cap-hAg-Kozak-sec-dNT-dmajor CSP construct-TMD-helper antigen-FI-A30L70;
cap-hAg-Kozak-Pfsec-dNT-dmajor CSP construct-helper antigen-FI-A30L70;
cap-hAg-Kozak-dNT-dmajor CSP construct-PfTMD-helper antigen-FI-A30L70;
cap-hAg-Kozak-Pfsec-dNT-dmajor CSP construct-PfTMD-helper antigen-FI-A30L70;
cap-hAg-Kozak-HSV-1gDsec-dNT-dmajor CSP construct-helper antigen-FI-A30L70;
cap-hAg-Kozak-dNT-dmajor CSP construct-HSV-1TMD-helper antigen-FI-A30L70;
cap-hAg-Kozak-HSV-1gDsec-dNT-dmajor CSP construct-HSV-1TMD-helper antigen-FI-A30L70;
cap-hAg-Kozak-HSV-1gDsec-dNT-dmajor CSP construct-PfTMD-helper antigen-FI-A30L70;
cap-hAg-Kozak-Pfsec-dNT-dmajor CSP construct-HSV-1TMD-helper antigen-FI-A30L70;
cap-hAg-Kozak-heterologoussec-dNT-dmajor CSP construct-helper antigen-FI-A30L70;
cap-hAg-Kozak-dNT-dmajor CSP construct-heterologousTMD-helper antigen-FI-A30L70;
cap-hAg-Kozak-heterologoussec-dNT-dmajor CSP construct-heterologousTMD-helper antigen-FI-A30L70;
cap-hAg-Kozak-dND CSP construct-FI-A30L70;
cap-hAg-Kozak-sec-dND CSP construct-FI-A30L70;
cap-hAg-Kozak-dND CSP construct-TMD-FI-A30L70;
cap-hAg-Kozak-sec-dND CSP construct-TMD-FI-A30L70;
cap-hAg-Kozak-Pfsec-dND CSP construct-FI-A30L70;
cap-hAg-Kozak-dND CSP construct-PfTMD-FI-A30L70;
cap-hAg-Kozak-Pfsec-dND CSP construct-PfTMD-FI-A30L70;
cap-hAg-Kozak-HSV-1gDsec-dND CSP construct-FI-A30L70;
cap-hAg-Kozak-dND CSP construct-HSV-1TMD-FI-A30L70;
cap-hAg-Kozak-HSV-1gDsec-dND CSP construct-HSV-1TMD-FI-A30L70;
cap-hAg-Kozak-HSV-1gDsec-dND CSP construct-PfTMD-FI-A30L70;
cap-hAg-Kozak-Pfsec-dND CSP construct-HSV-1TMD-FI-A30L70;
cap-hAg-Kozak-heterologoussec-dND CSP construct-FI-A30L70;
cap-hAg-Kozak-dND CSP construct-heterologousTMD-FI-A30L70;
cap-hAg-Kozak-heterologoussec-dND CSP construct-heterologousTMD-FI-A30L70;
cap-hAg-Kozak-dND-dmajor CSP construct-FI-A30L70;
cap-hAg-Kozak-sec-dND-dmajor CSP construct-FI-A30L70;
cap-hAg-Kozak-dND-dmajor CSP construct-TMD-FI-A30L70;
cap-hAg-Kozak-sec-dND-dmajor CSP construct-TMD-FI-A30L70;
cap-hAg-Kozak-Pfsec-dND-dmajor CSP construct-FI-A30L70;
cap-hAg-Kozak-dND-dmajor CSP construct-PfTMD-FI-A30L70;
cap-hAg-Kozak-Pfsec-dND-dmajor CSP construct-PfTMD-FI-A30L70;
cap-hAg-Kozak-HSV-1gDsec-dND-dmajor CSP construct-FI-A30L70;
cap-hAg-Kozak-dND-dmajor CSP construct-HSV-1TMD-FI-A30L70;
cap-hAg-Kozak-HSV-1gDsec-dND-dmajor CSP construct-HSV-1TMD-FI-A30L70;
cap-hAg-Kozak-HSV-1gDsec-dND-dmajor CSP construct-PfTMD-FI-A30L70;
cap-hAg-Kozak-Pfsec-dND-dmajor CSP construct-HSV-1TMD-FI-A30L70;
cap-hAg-Kozak-heterologoussec-dND-dmajor CSP construct-FI-A30L70;
cap-hAg-Kozak-dND-dmajor CSP construct-heterologousTMD-FI-A30L70;
cap-hAg-Kozak-heterologoussec-dND-dmajor CSP construct-heterologousTMD-FI-A30L70;
cap-hAg-Kozak-dND-dmajor-modJ CSP construct-FI-A30L70;
cap-hAg-Kozak-sec-dND-dmajor-modJ CSP construct-FI-A30L70;
cap-hAg-Kozak-dND-dmajor-modJ CSP construct-TMD-FI-A30L70;
cap-hAg-Kozak-sec-dND-dmajor-modJ CSP construct-TMD-FI-A30L70;
cap-hAg-Kozak-Pfsec-dND-dmajor-modJ CSP construct-FI-A30L70;
cap-hAg-Kozak-dND-dmajor-modJ CSP construct-PfTMD-FI-A30L70;
cap-hAg-Kozak-Pfsec-dND-dmajor-modJ CSP construct-PfTMD-FI-A30L70;
cap-hAg-Kozak-HSV-1gDsec-dND-dmajor-modJ CSP construct-FI-A30L70;
cap-hAg-Kozak-dND-dmajor-modJ CSP construct-HSV-1TMD-FI-A30L70;
cap-hAg-Kozak-HSV-1gDsec-dND-dmajor-modJ CSP construct-HSV-1TMD-FI-A30L70;
cap-hAg-Kozak-HSV-1gDsec-dND-dmajor-modJ CSP construct-PfTMD-FI-A30L70;
cap-hAg-Kozak-Pfsec-dND-dmajor-modJ CSP construct-HSV-1TMD-FI-A30L70;
cap-hAg-Kozak-heterologoussec-dND-dmajor-modJ CSP construct-FI-A30L70;
cap-hAg-Kozak-dND-dmajor-modJ CSP construct-heterologousTMD-FI-A30L70;
cap-hAg-Kozak-heterologoussec-dND-dmajor-modJ CSP construct-heterologousTMD-FI-A30L70;
cap-hAg-Kozak-pmajor-CT CSP construct-FI-A30L70;
cap-hAg-Kozak-sec-pmajor-CT CSP construct-FI-A30L70;
cap-hAg-Kozak-pmajor-CT CSP construct-TMD-FI-A30L70;
cap-hAg-Kozak-sec-pmajor-CT CSP construct-TMD-FI-A30L70;
cap-hAg-Kozak-Pfsec-pmajor-CT CSP construct-FI-A30L70;
cap-hAg-Kozak-pmajor-CT CSP construct-PfTMD-FI-A30L70;
cap-hAg-Kozak-Pfsec-pmajor-CT CSP construct-PfTMD-FI-A30L70;
cap-hAg-Kozak-HSV-1gDsec-pmajor-CT CSP construct-FI-A30L70;
cap-hAg-Kozak-pmajor-CT CSP construct-HSV-1TMD-FI-A30L70;
cap-hAg-Kozak-HSV-1gDsec-pmajor-CT CSP construct-HSV-1TMD-FI-A30L70;
cap-hAg-Kozak-HSV-1gDsec-pmajor-CT CSP construct-PfTMD-FI-A30L70;
cap-hAg-Kozak-Pfsec-pmajor-CT CSP construct-HSV-1TMD-FI-A30L70;
cap-hAg-Kozak-heterologoussec-pmajor-CT CSP construct-FI-A30L70;
cap-hAg-Kozak-pmajor-CT CSP construct-heterologousTMD-FI-A30L70; or
cap-hAg-Kozak-heterologoussec-pmajor-CT CSP construct-heterologousTMD-FI-A30L70.

In some embodiments, the different elements (see, Antigen, TMD) may be linked by one or more linkers, e.g., a linker selected from an amino acid sequence according to SEQ ID NO: 261, 267, 258, 276 (GGS), 279 (GGGS), 270, 282, 264, or 273. In some embodiments, a linker has the amino acid sequence according to SEQ ID NO: 258. In some embodiments, a linker has the amino acid sequence according to SEQ ID NO: 279 (GGGS). In some embodiments, a linker has the amino acid sequence according to SEQ ID NO: 270. In some embodiments, a linker has the amino acid sequence according to SEQ ID NO: 282.

In some embodiments, the sequence encoding a Plasmodium polypeptide construct described herein comprises a modified nucleoside replacing (partially or completely, preferably completely) uridine, wherein the modified nucleoside is selected from the group consisting of pseudouridine (w), N₁-methyl-pseudouridine (m1ψ), and 5-methyl-uridine.

In some embodiments, the sequence encoding a Plasmodium polypeptide construct described herein is codon-optimized.

In some embodiments, the G/C content of the sequence encoding a Plasmodium polypeptide construct described herein is increased compared to the wild type coding sequence.

In some embodiments, the RNA (in particular, mRNA) described herein comprises:
a 5′ UTR comprising the nucleotide sequence of SEQ ID NO: 415, or a nucleotide sequence having at least 99%, 98%, 97%, 96%, 95%, 90%, 85%, or 80% identity to the nucleotide sequence of SEQ ID NO: 415;
a 3′ UTR comprising the nucleotide sequence of SEQ ID NO: 416, or a nucleotide sequence having at least 99%, 98%, 97%, 96%, 95%, 90%, 85%, or 80% identity to the nucleotide sequence of SEQ ID NO: 416; and
a poly-A sequence comprising the nucleotide sequence of SEQ ID NO: 417.

In some embodiments, the RNA (in particular, mRNA) described herein comprises:
m₂^{7,3}′—OGppp(m¹²′—O) ApG as capping structure at the 5′-end of the mRNA;
a 5′ UTR comprising the nucleotide sequence of SEQ ID NO: 415, or a nucleotide sequence having at least 99%, 98%, 97%, 96%, 95%, 90%, 85%, or 80% identity to the nucleotide sequence of SEQ ID NO: 415;
a 3′ UTR comprising the nucleotide sequence of SEQ ID NO: 416, or a nucleotide sequence having at least 99%, 98%, 97%, 96%, 95%, 90%, 85%, or 80% identity to the nucleotide sequence of SEQ ID NO: 416; and
a poly-A sequence comprising the nucleotide sequence of SEQ ID NO: 417.

In some embodiments, the RNA is unmodified. In some embodiments, the RNA is modified. In some embodiments, the RNA comprises N₁-methyl-pseudouridine (m1ψ) in place of at least one uridine (e.g., in place of each uridine).

In some embodiments, the RNA (in particular, mRNA) described herein comprises:
m₂^{7,3}′—OGppp(m¹²′—O) ApG as capping structure at the 5′-end of the mRNA;
a 5′ UTR comprising the nucleotide sequence of SEQ ID NO: 415, or a nucleotide sequence having at least 99%, 98%, 97%, 96%, 95%, 90%, 85%, or 80% identity to the nucleotide sequence of SEQ ID NO: 415;
a 3′ UTR comprising the nucleotide sequence of SEQ ID NO: 416, or a nucleotide sequence having at least 99%, 98%, 97%, 96%, 95%, 90%, 85%, or 80% identity to the nucleotide sequence of SEQ ID NO: 416; and
a poly-A sequence comprising the nucleotide sequence of SEQ ID NO: 417; and
   N₁-methyl-pseudouridine (m1ψ) in place of at least one uridine (e.g., in place of each uridine).

In some embodiments, a Plasmodium polypeptide construct described herein includes one or more malarial polypeptides or portions thereof from Plasmodium falciparum.

In some embodiments, a Plasmodium polypeptide construct described herein includes one or more regions or portions thereof derived from a Plasmodium falciparum CSP protein, an immunogenic variant thereof, or an immunogenic fragment of the Plasmodium falciparum CSP protein or the immunogenic variant thereof. Thus, in some embodiments, the RNA, e.g., mRNA, used in the present disclosure encodes an amino acid sequence comprising an Plasmodium falciparum CSP protein, an immunogenic variant thereof, or an immunogenic fragment of the Plasmodium falciparum CSP protein or the immunogenic variant thereof.

In some embodiments, RNA (in particular, mRNA) described herein (e.g., contained in the compositions/formulations of the present disclosure and/or used in the methods of the present disclosure) may be presented as a product containing the vaccine RNA as active substance and other ingredients comprising: ALC-0315 ((4-hydroxybutyl) azanediyl)bis(hexane-6,1-diyl)bis(2-hexyldecanoate), ALC-0159 (2-[(polyethylene glycol)-2000]—N,N-ditetradecylacetamide), 1,2-Distearoyl-sn-glycero-3-phosphocholine (DSPC), and cholesterol.

In some embodiments, the RNA (in particular, mRNA) described herein is formulated or is to be formulated as a liquid, a solid, or a combination thereof.

In some embodiments, the RNA (in particular, mRNA) described herein is formulated or is to be formulated for injection.

In some embodiments, the RNA (in particular, mRNA) described herein is formulated or is to be formulated for intramuscular administration.

In some embodiments, the RNA (in particular, mRNA) described herein is formulated or is to be formulated as a composition, e.g., a pharmaceutical composition.

In some embodiments, the composition comprises a cationically ionizable lipid.

In some embodiments, the composition comprises a cationically ionizable lipid and one or more additional lipids. In some embodiments, the one or more additional lipids are selected from polymer-conjugated lipids, neutral lipids, and combinations thereof. In some embodiments, the neutral lipids include phospholipids, steroid lipids, and combinations thereof. In some embodiments, the one or more additional lipids are a combination of a polymer-conjugated lipid, a phospholipid, and a steroid lipid.

In some embodiments, the composition comprises a cationically ionizable lipid; a polymer-conjugated lipid which is a PEG-conjugated lipid; cholesterol; and a phospholipid. In some embodiments, the phospholipid is DSPC. In some embodiments, the phospholipid is DOPE.

In some embodiments, the composition comprises a cationically ionizable lipid; a polymer-conjugated lipid which is 2-[(polyethylene glycol)-2000]—N,N-ditetradecylacetamide; cholesterol; and a phospholipid. In some embodiments, the phospholipid is DSPC. In some embodiments, the phospholipid is DOPE.

In some embodiments, the composition comprises a cationically ionizable lipid which is ((4-hydroxybutyl) azanediyl)bis(hexane-6,1-diyl)bis(2-hexyldecanoate); a polymer-conjugated lipid which is 2-[(polyethylene glycol)-2000]—N,N-ditetradecylacetamide; cholesterol; and a phospholipid. In some embodiments, the phospholipid is DSPC. In some embodiments, the phospholipid is DOPE. In some embodiments, at least a portion of (i) the RNA, (ii) the cationically ionizable lipid, and if present, (iii) the one or more additional lipids is present in particles. In some embodiments, the particles are nanoparticles, such as lipid nanoparticles (LNPs).

In some embodiments, the composition, in particular the pharmaceutical composition, is a vaccine.

In some embodiments, the composition, in particular the pharmaceutical composition, further comprises one or more pharmaceutically acceptable carriers, diluents and/or excipients.

In some embodiments, the RNA and/or the composition, in particular the pharmaceutical composition, is/are a component of a kit.

In some embodiments, the kit further comprises instructions for use of the RNA for inducing an immune response against Plasmodium falciparum in a subject.

In some embodiments, the kit further comprises instructions for use of the RNA for therapeutically or prophylactically treating a Plasmodium falciparum infection in a subject.

In some embodiments, the subject is a human.

In some embodiments, the RNA (in particular, mRNA), e.g., RNA encoding a Plasmodium polypeptide construct, described in the present disclosure is non-immunogenic. RNA encoding an immunostimulant may be administered according to the present disclosure to provide an adjuvant effect. The RNA encoding an immunostimulant may be standard RNA or non-immunogenic RNA.

### F. Combinations of Polyribonucleotides

The present disclosure, among other things, utilizes RNA technologies as a modality to express two or more polypeptide constructs. In some embodiments, two or more different polypeptide constructs are two or more Plasmodium polypeptide constructs. As described further herein, in some embodiments, a Plasmodium polypeptide construct includes one or more malarial proteins, or one or more portions thereof.

In some embodiments, two or more Plasmodium polypeptide constructs include a first Plasmodium polypeptide construct and a second Plasmodium polypeptide construct. In some embodiments, a first Plasmodium polypeptide construct comprises one or more Plasmodium CSP polypeptide regions or portions thereof (e.g., immunogenic fragments of Plasmodium CSP). In some embodiments, such a first Plasmodium polypeptide construct additionally includes one or more additional amino acid sequences, such as a secretory signal (e.g., a heterologous secretory signal), a transmembrane region (e.g., a heterologous transmembrane region), a helper antigen, a multimerization region, and/or a linker, as described herein.

In some embodiments, a second Plasmodium polypeptide construct is different than a first Plasmodium polypeptide construct. In some embodiments, a second Plasmodium polypeptide construct includes one or more Plasmodium polypeptide regions or portions thereof (e.g., immunogenic fragments of a Plasmodium polypeptide), wherein the one or more Plasmodium polypeptide regions or portions thereof are different than one or more Plasmodium CSP polypeptide regions or portions thereof included in a first Plasmodium polypeptide construct. While the one or more Plasmodium polypeptide regions or portions thereof are different than one or more Plasmodium CSP polypeptide regions or portions thereof included in a first Plasmodium polypeptide construct, such that the second Plasmodium polypeptide construct is not identical to the first Plasmodium polypeptide construct, the present disclosure contemplates that there may be some Plasmodium polypeptide regions or portions thereof that are common to the first and second Plasmodium polypeptide constructs. For example, if a first malarial construct comprises a Plasmodium CSP major repeat region portion and a Plasmodium CSP C-terminal region, a second malarial construct can comprise, among other Plasmodium polypeptide regions or portions thereof, a Plasmodium CSP C-terminal region. In some embodiments, a second Plasmodium polypeptide construct additionally includes one or more additional amino acid sequences, such as a secretory signal (e.g., a heterologous secretory signal), a transmembrane region (e.g., a heterologous transmembrane region), a helper antigen, a multimerization region, and/or a linker, as described herein.

In certain embodiments, a first Plasmodium polypeptide construct comprises one or more Plasmodium CSP polypeptide regions or portions thereof (e.g., immunogenic fragments of Plasmodium CSP), and a second Plasmodium polypeptide construct includes one or more Plasmodium polypeptide regions or portions thereof, wherein the one or more Plasmodium polypeptide regions or portions thereof comprise one or more Plasmodium T-cell antigens.

In accordance with the above, the present disclosure provides two or more polyribonucleotides that each encode a polypeptide construct. In some embodiments, two or more polyribonucleotides that each encode a Plasmodium polypeptide construct. As described further herein, in some embodiments, a Plasmodium polypeptide construct includes one or more malarial proteins, or one or more portions thereof.

In some embodiments, a combination of two or more polyribonucleotides each encode a Plasmodium polypeptide construct. For instance, in some embodiments, a combination as provided herein can include (i) a first polyribonucleotide, wherein the first polyribonucleotide encodes a first polypeptide, and the first polypeptide comprises one or more Plasmodium CSP polypeptide regions or portions thereof, as described herein; and (ii) a second polyribonucleotide, wherein the second polyribonucleotide encodes a second polypeptide, and the second polypeptide comprises one or more Plasmodium polypeptide regions or portions thereof.

In some embodiments, a combination as provided herein can include (i) a first polyribonucleotide, wherein the first polyribonucleotide encodes a first polypeptide, and the first polypeptide comprises one or more Plasmodium CSP polypeptide regions or portions thereof, as described herein; and (ii) a second polyribonucleotide, where the second polyribonucleotide encodes a second polypeptide, and the second polypeptide comprises one or more Plasmodium T-cell antigens.

In some embodiments, a first and second polyribonucleotide, as described herein, encode a Plasmodium polypeptide construct described herein has one of the following structures:
cap-hAg-Kozak-Antigen-FI-A30L70;
cap-hAg-Kozak-sec-Antigen-FI-A30L70;
cap-hAg-Kozak-Antigen-TMD-FI-A30L70; or
cap-hAg-Kozak-sec-Antigen-TMD-FI-A30L70.

In some embodiments, hAg-Kozak comprises the nucleotide sequence of SEQ ID NO: 415. In some embodiments, FI comprises the nucleotide sequence of SEQ ID NO: 416. In some embodiments, A30L70 comprises the nucleotide sequence of SEQ ID NO: 417.

In some embodiments, the secretory signal is from Plasmodium falciparum, preferably Plasmodium falciparum isolate 3D7, and referred to herein as Pfsec and has the sequence as defined in SEQ ID NO: 174. In some embodiments, the secretory signal is heterologous and selected from the amino acid sequences as provided in Table 3. In some embodiments, the secretory signal is HSV1-gD and referred to herein as HSV-1gDsec and has the sequence as defined in SEQ ID NO: 159, SEQ ID NO: 162, or SEQ ID NO: 165.

In some embodiments, the TMD is a glycosylphosphatidylinositol (GPI) anchor region from Plasmodium CSP, preferably from Plasmodium falciparum isolate 3D7, and referred to herein as PfTMD and has the amino acid sequence of SEQ ID NO. 231. In some embodiments, the TMD is heterologous. In some embodiments, the TMD is from HSV1-gD and referred to herein as HSV-1TMD and has the amino acid sequence of SEQ ID NO:234.

In some embodiments, the Antigen comprises a full-length CSP construct as defined above, a N-terminal region deletion CSP construct as defined above, a N-terminal region and major region deleted CSP construct as defined above, a N-terminal domain deleted CSP construct as defined above, a N-terminal domain and major repeat region deleted CSP construct as defined above, a N-terminal domain and major repeat region deleted CSP construct with modified junction region variants or portions as defined above or a major repeat region portion and C-terminal region containing CSP constructs as defined above. In some embodiments, the Antigen comprises one or more Plasmodium T-cell antigens.

In some embodiments, the different elements (see, Antigen, TMD) of a first polypeptide may be linked by one or more linkers, e.g., a linker selected from an amino acid sequence according to SEQ ID NO: 261, 267, 258, 276, 279, 270, 282, 264, or 273. In some embodiments, a linker has an amino acid sequence according to SEQ ID NO: 258. In some embodiments, a linker has an amino acid sequence according to SEQ ID NO: 279. In some embodiments, a linker has an amino acid sequence according to SEQ ID NO: 270. In some embodiments, a linker has an amino acid sequence according to SEQ ID NO: 282.

In some embodiments, a combination as provided herein can include (i) a first polyribonucleotide, where the first polyribonucleotide encodes a first polypeptide; and (ii) a second polyribonucleotide, where the second polyribonucleotide encodes a second polypeptide, and the second polypeptide comprises one or more Plasmodium polypeptide regions or portions thereof. In some embodiments, a combination as provided herein can include (i) a first polyribonucleotide, where the first polyribonucleotide encodes a first polypeptide; and (ii) a second polyribonucleotide, where the second polyribonucleotide encodes a second polypeptide, and the second polypeptide comprises one or more Plasmodium T-cell antigens. In some embodiments, the first polyribonucleotide has one of the following structures:
cap-hAg-Kozak-full-length CSP construct-FI-A30L70;
cap-hAg-Kozak-sec-full-length CSP construct-FI-A30L70;
cap-hAg-Kozak-full-length CSP construct-TMD-FI-A30L70;
cap-hAg-Kozak-sec-full-length CSP construct-TMD-FI-A30L70;
cap-hAg-Kozak-Pfsec-full-length CSP construct-FI-A30L70;
cap-hAg-Kozak-full-length CSP construct-PfTMD-FI-A30L70;
cap-hAg-Kozak-Pfsec-full-length CSP construct-PfTMD-FI-A30L70;
cap-hAg-Kozak-HSV-1gDsec-full-length CSP construct-FI-A30L70;
cap-hAg-Kozak-full-length CSP construct-HSV-1TMD-FI-A30L70;
cap-hAg-Kozak-HSV-1gDsec-full-length CSP construct-HSV-1TMD-FI-A30L70;
cap-hAg-Kozak-Pfsec-full-length CSP construct-HSV-1TMD-FI-A30L70;
cap-hAg-Kozak-HSV-1gDsec-full-length CSP construct-PfTMD-FI-A30L70;
cap-hAg-Kozak-heterologoussec-full-length CSP construct-FI-A30L70;
cap-hAg-Kozak-full-length CSP construct-heterologousTMD-FI-A30L70;
cap-hAg-Kozak-heterologoussec-full-length CSP construct-heterologousTMD-FI-A30L70;
cap-hAg-Kozak-dNT CSP construct-FI-A30L70;
cap-hAg-Kozak-sec-dNT CSP construct-FI-A30L70;
cap-hAg-Kozak-dNT CSP construct-TMD-FI-A30L70;
cap-hAg-Kozak-sec-dNT CSP construct-TMD-FI-A30L70;
cap-hAg-Kozak-Pfsec-dNT CSP construct-FI-A30L70;
cap-hAg-Kozak-dNT CSP construct-PfTMD-FI-A30L70;
cap-hAg-Kozak-Pfsec-dNT CSP construct-PfTMD-FI-A30L70;
cap-hAg-Kozak-HSV-1gDsec-dNT CSP construct-FI-A30L70;
cap-hAg-Kozak-dNT CSP construct-HSV-1TMD-FI-A30L70;
cap-hAg-Kozak-HSV-1gDsec-dNT CSP construct-HSV-1TMD-FI-A30L70;
cap-hAg-Kozak-HSV-1gDsec-dNT CSP construct-PfTMD-FI-A30L70;
cap-hAg-Kozak-Pfsec-dNT CSP construct-HSV-1TMD-FI-A30L70;
cap-hAg-Kozak-heterologoussec-dNT CSP construct-FI-A30L70;
cap-hAg-Kozak-dNT CSP construct-heterologousTMD-FI-A30L70;
cap-hAg-Kozak-heterologoussec-dNT CSP construct-heterologousTMD-FI-A30L70;
cap-hAg-Kozak-dNT-dmajor CSP construct-FI-A30L70;
cap-hAg-Kozak-sec-dNT-dmajor CSP construct-FI-A30L70;
cap-hAg-Kozak-dNT-dmajor CSP construct-TMD-FI-A30L70;
cap-hAg-Kozak-sec-dNT-dmajor CSP construct-TMD-FI-A30L70;
cap-hAg-Kozak-Pfsec-dNT-dmajor CSP construct-FI-A30L70;
cap-hAg-Kozak-dNT-dmajor CSP construct-PfTMD-FI-A30L70;
cap-hAg-Kozak-Pfsec-dNT-dmajor CSP construct-PfTMD-FI-A30L70;
cap-hAg-Kozak-HSV-1gDsec-dNT-dmajor CSP construct-FI-A30L70;
cap-hAg-Kozak-dNT-dmajor CSP construct-HSV-1TMD-FI-A30L70;
cap-hAg-Kozak-HSV-1gDsec-dNT-dmajor CSP construct-HSV-1TMD-FI-A30L70;
cap-hAg-Kozak-HSV-1gDsec-dNT-dmajor CSP construct-PfTMD-FI-A30L70;
cap-hAg-Kozak-Pfsec-dNT-dmajor CSP construct-HSV-1TMD-FI-A30L70;
cap-hAg-Kozak-heterologoussec-dNT-dmajor CSP construct-FI-A30L70;
cap-hAg-Kozak-dNT-dmajor CSP construct-heterologousTMD-FI-A30L70;
cap-hAg-Kozak-heterologoussec-dNT-dmajor CSP construct-heterologousTMD-FI-A30L70;
cap-hAg-Kozak-dNT-dmajor CSP construct-helper antigen-FI-A30L70;
cap-hAg-Kozak-sec-dNT-dmajor CSP construct-helper antigen-FI-A30L70;
cap-hAg-Kozak-dNT-dmajor CSP construct-TMD-helper antigen-FI-A30L70;
cap-hAg-Kozak-sec-dNT-dmajor CSP construct-TMD-helper antigen-FI-A30L70;
cap-hAg-Kozak-Pfsec-dNT-dmajor CSP construct-helper antigen-FI-A30L70;
cap-hAg-Kozak-dNT-dmajor CSP construct-PTMD-helper antigen-FI-A30L70;
cap-hAg-Kozak-Pfsec-dNT-dmajor CSP construct-PfTMD-helper antigen-FI-A30L70;
cap-hAg-Kozak-HSV-1gDsec-dNT-dmajor CSP construct-helper antigen-FI-A30L70;
cap-hAg-Kozak-dNT-dmajor CSP construct-HSV-1TMD-helper antigen-FI-A30L70;
cap-hAg-Kozak-HSV-1gDsec-dNT-dmajor CSP construct-HSV-1TMD-helper antigen-FI-A30L70;
cap-hAg-Kozak-HSV-1gDsec-dNT-dmajor CSP construct-PfTMD-helper antigen-FI-A30L70;
cap-hAg-Kozak-Pfsec-dNT-dmajor CSP construct-HSV-1TMD-helper antigen-FI-A30L70;
cap-hAg-Kozak-heterologoussec-dNT-dmajor CSP construct-helper antigen-FI-A30L70;
cap-hAg-Kozak-dNT-dmajor CSP construct-heterologousTMD-helper antigen-FI-A30L70;
cap-hAg-Kozak-heterologoussec-dNT-dmajor CSP construct-heterologousTMD-helper antigen-FI-A30L70;
cap-hAg-Kozak-dND CSP construct-FI-A30L70;
cap-hAg-Kozak-sec-dND CSP construct-FI-A30L70;
cap-hAg-Kozak-dND CSP construct-TMD-FI-A30L70;
cap-hAg-Kozak-sec-dND CSP construct-TMD-FI-A30L70;
cap-hAg-Kozak-Pfsec-dND CSP construct-FI-A30L70;
cap-hAg-Kozak-dND CSP construct-PfTMD-FI-A30L70;
cap-hAg-Kozak-Pfsec-dND CSP construct-PTMD-FI-A30L70;
cap-hAg-Kozak-HSV-1gDsec-dND CSP construct-FI-A30L70;
cap-hAg-Kozak-dND CSP construct-HSV-1TMD-FI-A30L70;
cap-hAg-Kozak-HSV-1gDsec-dND CSP construct-HSV-1TMD-FI-A30L70;
cap-hAg-Kozak-HSV-1gDsec-dND CSP construct-PfTMD-FI-A30L70;
cap-hAg-Kozak-Pfsec-dND CSP construct-HSV-1TMD-FI-A30L70;
cap-hAg-Kozak-heterologoussec-dND CSP construct-FI-A30L70;
cap-hAg-Kozak-dND CSP construct-heterologousTMD-FI-A30L70;
cap-hAg-Kozak-heterologoussec-dND CSP construct-heterologousTMD-FI-A30L70;
cap-hAg-Kozak-dND-dmajor CSP construct-FI-A30L70;
cap-hAg-Kozak-sec-dND-dmajor CSP construct-FI-A30L70;
cap-hAg-Kozak-dND-dmajor CSP construct-TMD-FI-A30L70;
cap-hAg-Kozak-sec-dND-dmajor CSP construct-TMD-FI-A30L70;
cap-hAg-Kozak-Pfsec-dND-dmajor CSP construct-FI-A30L70;
cap-hAg-Kozak-dND-dmajor CSP construct-PfTMD-FI-A30L70;
cap-hAg-Kozak-Pfsec-dND-dmajor CSP construct-PfTMD-FI-A30L70;
cap-hAg-Kozak-HSV-1gDsec-dND-dmajor CSP construct-FI-A30L70;
cap-hAg-Kozak-dND-dmajor CSP construct-HSV-1TMD-FI-A30L70;
cap-hAg-Kozak-HSV-1gDsec-dND-dmajor CSP construct-HSV-1TMD-FI-A30L70;
cap-hAg-Kozak-HSV-1gDsec-dND-dmajor CSP construct-PfTMD-FI-A30L70;
cap-hAg-Kozak-Pfsec-dND-dmajor CSP construct-HSV-1TMD-FI-A30L70;
cap-hAg-Kozak-heterologoussec-dND-dmajor CSP construct-FI-A30L70;
cap-hAg-Kozak-dND-dmajor CSP construct-heterologousTMD-FI-A30L70;
cap-hAg-Kozak-heterologoussec-dND-dmajor CSP construct-heterologousTMD-FI-A30L70;
cap-hAg-Kozak-dND-dmajor-modJ CSP construct-FI-A30L70;
cap-hAg-Kozak-sec-dND-dmajor-modJ CSP construct-FI-A30L70;
cap-hAg-Kozak-dND-dmajor-modJ CSP construct-TMD-FI-A30L70;
cap-hAg-Kozak-sec-dND-dmajor-modJ CSP construct-TMD-FI-A30L70;
cap-hAg-Kozak-Pfsec-dND-dmajor-modJ CSP construct-FI-A30L70;
cap-hAg-Kozak-dND-dmajor-modJ CSP construct-PfTMD-FI-A30L70;
cap-hAg-Kozak-Pfsec-dND-dmajor-modJ CSP construct-PfTMD-FI-A30L70;
cap-hAg-Kozak-HSV-1gDsec-dND-dmajor-modJ CSP construct-FI-A30L70;
cap-hAg-Kozak-dND-dmajor-modJ CSP construct-HSV-1TMD-FI-A30L70;
cap-hAg-Kozak-HSV-1gDsec-dND-dmajor-modJ CSP construct-HSV-1TMD-FI-A30L70;
cap-hAg-Kozak-HSV-1gDsec-dND-dmajor-modJ CSP construct-PfTMD-FI-A30L70;
cap-hAg-Kozak-Pfsec-dND-dmajor-modJ CSP construct-HSV-1TMD-FI-A30L70;
cap-hAg-Kozak-heterologoussec-dND-dmajor-modJ CSP construct-FI-A30L70;
cap-hAg-Kozak-dND-dmajor-modJ CSP construct-heterologousTMD-FI-A30L70;
cap-hAg-Kozak-heterologoussec-dND-dmajor-modJ CSP construct-heterologousTMD-FI-A30L70;
cap-hAg-Kozak-pmajor-CT CSP construct-FI-A30L70;
cap-hAg-Kozak-sec-pmajor-CT CSP construct-FI-A30L70;
cap-hAg-Kozak-pmajor-CT CSP construct-TMD-FI-A30L70;
cap-hAg-Kozak-sec-pmajor-CT CSP construct-TMD-FI-A30L70;
cap-hAg-Kozak-Pfsec-pmajor-CT CSP construct-FI-A30L70;
cap-hAg-Kozak-pmajor-CT CSP construct-PfTMD-FI-A30L70;
cap-hAg-Kozak-Pfsec-pmajor-CT CSP construct-PfTMD-FI-A30L70;
cap-hAg-Kozak-HSV-1gDsec-pmajor-CT CSP construct-FI-A30L70;
cap-hAg-Kozak-pmajor-CT CSP construct-HSV-1TMD-FI-A30L70;
cap-hAg-Kozak-HSV-1gDsec-pmajor-CT CSP construct-HSV-1TMD-FI-A30L70;
cap-hAg-Kozak-HSV-1gDsec-pmajor-CT CSP construct-PfTMD-FI-A30L70;
cap-hAg-Kozak-Pfsec-pmajor-CT CSP construct-HSV-1TMD-FI-A30L70;
cap-hAg-Kozak-heterologoussec-pmajor-CT CSP construct-FI-A30L70;
cap-hAg-Kozak-pmajor-CT CSP construct-heterologousTMD-FI-A30L70; or cap-hAg-Kozak-heterologoussec-pmajor-CT CSP construct-heterologousTMD-FI-A30L70.

In some embodiments, a first and/or a second polyribonucleotide described herein comprises a modified nucleoside replacing (partially or completely, preferably completely) uridine, wherein the modified nucleoside is selected from the group consisting of pseudouridine (w), N₁-methyl-pseudouridine (m1ψ), and 5-methyl-uridine.

In some embodiments, a first and/or a second polyribonucleotide described herein is codon-optimized.

In some embodiments, the G/C content of a first and/or a second polyribonucleotide described herein is increased compared to the wild type coding sequence.

In some embodiments, a first and/or a second polyribonucleotide described herein comprises:
a 5′ UTR comprising the nucleotide sequence of SEQ ID NO: 415, or a nucleotide sequence having at least 99%, 98%, 97%, 96%, 95%, 90%, 85%, or 80% identity to the nucleotide sequence of SEQ ID NO: 415;
a 3′ UTR comprising the nucleotide sequence of SEQ ID NO: 416, or a nucleotide sequence having at least 99%, 98%, 97%, 96%, 95%, 90%, 85%, or 80% identity to the nucleotide sequence of SEQ ID NO: 416; and
a poly-A sequence comprising the nucleotide sequence of SEQ ID NO: 417.

In some embodiments, a first and/or a second polyribonucleotide described herein comprises:
m₂^{7,3}′—OGppp(m12′—O) ApG as capping structure at the 5′-end of the polyribonucleotide;
a 5′ UTR comprising the nucleotide sequence of SEQ ID NO: 415, or a nucleotide sequence having at least 99%, 98%, 97%, 96%, 95%, 90%, 85%, or 80% identity to the nucleotide sequence of SEQ ID NO: 415;
a 3′ UTR comprising the nucleotide sequence of SEQ ID NO: 416, or a nucleotide sequence having at least 99%, 98%, 97%, 96%, 95%, 90%, 85%, or 80% identity to the nucleotide sequence of SEQ ID NO: 416; and
a poly-A sequence comprising the nucleotide sequence of SEQ ID NO: 417.

In some embodiments, a first and/or a second polyribonucleotide described herein is unmodified. In some embodiments, a first and/or a second polyribonucleotide described herein is modified. In some embodiments, a first and/or a second polyribonucleotide described herein comprises N₁-methyl-pseudouridine (m1ψ) in place of at least one uridine (e.g., in place of each uridine).

In some embodiments, a first and/or a second polyribonucleotide described herein comprises:
m₂^{7,3}′—OGppp(m12′—O) ApG as capping structure at the 5′-end of the mRNA;
a 5′ UTR comprising the nucleotide sequence of SEQ ID NO: 415, or a nucleotide sequence having at least 99%, 98%, 97%, 96%, 95%, 90%, 85%, or 80% identity to the nucleotide sequence of SEQ ID NO: 415;
a 3′ UTR comprising the nucleotide sequence of SEQ ID NO: 416, or a nucleotide sequence having at least 99%, 98%, 97%, 96%, 95%, 90%, 85%, or 80% identity to the nucleotide sequence of SEQ ID NO: 416;
a poly-A sequence comprising the nucleotide sequence of SEQ ID NO: 417; and
N₁-methyl-pseudouridine (m1ψ) in place of at least one uridine (e.g., in place of each uridine).

In some embodiments, a combination described above can be administered in a pharmaceutical composition as described herein. In some embodiments, two or more polyribonucleotides of a combination as described above can be administered in separate pharmaceutical compositions as described herein. For example, in some embodiments, a combination provided herein comprises (i) a first pharmaceutical composition comprising a first polyribonucleotide, wherein the first polyribonucleotide encodes a first polypeptide, and the first polypeptide comprises one or more Plasmodium CSP polypeptide regions or portions thereof; and (ii) a second pharmaceutical composition comprising a second polyribonucleotide, wherein the second polyribonucleotide encodes a second polypeptide, and the second polypeptide comprises one or more polypeptide regions or portions thereof. In some embodiments, a combination provided herein comprises (i) a first pharmaceutical composition comprising a first polyribonucleotide, wherein the first polyribonucleotide encodes a first polypeptide, and the first polypeptide comprises one or more Plasmodium CSP polypeptide regions or portions thereof; and (ii) a second pharmaceutical composition comprising a second polyribonucleotide, wherein the second polyribonucleotide encodes a second polypeptide, and the second polypeptide comprises one or more Plasmodium T-cell antigens.

In some embodiments, a first or a second polyribonucleotide as described herein (e.g., contained in the compositions/formulations of the present disclosure and/or used in the methods of the present disclosure) may be presented as a product containing the first or the second polyribonucleotide as described herein as active substance and other ingredients comprising: ALC-0315 ((4-hydroxybutyl) azanediyl)bis(hexane-6,1-diyl)bis(2-hexyldecanoate), ALC-0159 (2-[(polyethylene glycol)-2000]—N,N-ditetradecylacetamide), 1,2-Distearoyl-sn-glycero-3-phosphocholine (DSPC), and cholesterol.

In some embodiments, a first or a second polyribonucleotide as described herein is formulated or is to be formulated as a liquid, a solid, or a combination thereof.

In some embodiments, a first or a second polyribonucleotide as described herein is formulated or is to be formulated for injection.

In some embodiments, a first or a second polyribonucleotide as described herein is formulated or is to be formulated for intramuscular administration.

In some embodiments, a first or a second polyribonucleotide as described herein is formulated or is to be formulated as a composition, e.g., a pharmaceutical composition.

In some embodiments, a composition comprises a cationically ionizable lipid.

In some embodiments, a composition comprises a cationically ionizable lipid and one or more additional lipids. In some embodiments, one or more additional lipids are selected from polymer-conjugated lipids, neutral lipids, and combinations thereof. In some embodiments, neutral lipids include phospholipids, steroid lipids, and combinations thereof. In some embodiments, one or more additional lipids are a combination of a polymer-conjugated lipid, a phospholipid, and a steroid lipid.

In some embodiments, a composition comprises a cationically ionizable lipid; a polymer-conjugated lipid which is a PEG-conjugated lipid; cholesterol; and a phospholipid. In some embodiments, a phospholipid is DSPC. In some embodiments, a phospholipid is DOPE.

In some embodiments, a composition comprises a cationically ionizable lipid; a polymer-conjugated lipid which is 2-[(polyethylene glycol)-2000]—N,N-ditetradecylacetamide; cholesterol; and a phospholipid. In some embodiments, a phospholipid is DSPC. In some embodiments, a phospholipid is DOPE.

In some embodiments, a composition comprises a cationically ionizable lipid which is ((4-hydroxybutyl) azanediyl)bis(hexane-6,1-diyl)bis(2-hexyldecanoate); a polymer-conjugated lipid which is 2-[(polyethylene glycol)-2000]—N,N-ditetradecylacetamide; cholesterol; and a phospholipid. In some embodiments, a phospholipid is DSPC. In some embodiments, a phospholipid is DOPE. In some embodiments, at least a portion of (i) a first or a second polyribonucleotide as described herein, (ii) a cationically ionizable lipid, and if present, (iii) one or more additional lipids is present in particles. In some embodiments, particles are nanoparticles, such as lipid nanoparticles (LNPs).

In some embodiments, a composition, in particular the pharmaceutical composition, is a vaccine.

In some embodiments, a composition, in particular the pharmaceutical composition, further comprises one or more pharmaceutically acceptable carriers, diluents and/or excipients.

### IV. RNA Delivery Technologies

Provided polyribonucleotides may be delivered for therapeutic applications described herein using any appropriate methods known in the art, including, e.g., delivery as naked RNAs, or delivery mediated by viral and/or non-viral vectors, polymer-based vectors, lipid compositions, nanoparticles (e.g., lipid nanoparticles, polymeric nanoparticles, lipid-polymer hybrid nanoparticles, etc.), and/or peptide-based vectors. See, e.g., Wadhwa et al. “Opportunities and Challenges in the Delivery of mRNA-Based Vaccines” Pharmaceutics (2020) 102 (27 pages), the content of which is incorporated herein by reference, for information on various approaches that may be useful for delivery polyribonucleotides described herein.

In some embodiments, one or more polyribonucleotides can be formulated with lipid nanoparticles for delivery (e.g., administration).

In some embodiments, lipid nanoparticles can be designed to protect polyribonucleotides from extracellular RNases and/or engineered for systemic delivery of the RNA to target cells (e.g., liver cells). In some embodiments, such lipid nanoparticles may be particularly useful to deliver polyribonucleotides when polyribonucleotides are intravenously or intramuscularly administered to a subject.

### A. Lipid Compositions

### 1 Lipids and Lipid-Like Materials

The terms “lipid” and “lipid-like material” are broadly defined herein as molecules which comprise one or more hydrophobic moieties or groups and optionally also one or more hydrophilic moieties or groups. Molecules comprising hydrophobic moieties and hydrophilic moieties are also frequently denoted as amphiphiles. Lipids are usually poorly soluble in water. In an aqueous environment, the amphiphilic nature allows the molecules to self-assemble into organized structures and different phases. One of those phases consists of lipid bilayers, as they are present in vesicles, multilamellar/unilamellar liposomes, or membranes in an aqueous environment. Hydrophobicity can be conferred by the inclusion of a polar groups that include, but are not limited to, long-chain saturated and unsaturated aliphatic hydrocarbon groups and such groups substituted by one or more aromatic, cycloaliphatic, or heterocyclic group(s). The hydrophilic groups may comprise polar and/or charged groups and include carbohydrates, phosphate, carboxylic, sulfate, amino, sulfhydryl, nitro, hydroxyl, and other like groups.

Often, an amphiphilic compound has a polar head attached to a long hydrophobic tail. In some embodiments, the polar portion is soluble in water, while the non-polar portion is insoluble in water. In addition, the polar portion may have either a formal positive charge, or a formal negative charge. Alternatively, the polar portion may have both a formal positive and a negative charge, and be a zwitterion or inner salt. For purposes of the disclosure, the amphiphilic compound can be, but is not limited to, one or a plurality of natural or non-natural lipids and lipid-like compounds.

A “lipid-like material” is a substance that is structurally and/or functionally related to a lipid but may not be considered a lipid in a strict sense. For example, the term includes compounds that are able to form amphiphilic layers as they are present in vesicles, multilamellar/unilamellar liposomes, or membranes in an aqueous environment and includes surfactants, or synthesized compounds with both hydrophilic and hydrophobic moieties. Generally speaking, the term refers to molecules, which comprise hydrophilic and hydrophobic moieties with different structural organization, which may or may not be similar to that of lipids.

Specific examples of amphiphilic compounds that may be included in an amphiphilic layer include, but are not limited to, phospholipids, aminolipids and sphingolipids.

Generally, lipids may be divided into eight categories: fatty acids, glycerolipids, glycerophospholipids, sphingolipids, saccharolipids, polyketides (derived from condensation of ketoacyl subunits), sterols and prenol lipids (derived from condensation of isoprene subunits). Although the term “lipid” is sometimes used as a synonym for fats, fats are a subgroup of lipids called triglycerides. Lipids also encompass molecules such as fatty acids and their derivatives (including tri-, di-, monoglycerides, and phospholipids), as well as sterol-containing metabolites such as cholesterol.

Fatty acids are a diverse group of molecules made of a hydrocarbon chain that terminates with a carboxylic acid group; this arrangement confers the molecule with a polar, hydrophilic end, and a nonpolar, hydrophobic end that is insoluble in water. The carbon chain, typically between four and 24 carbons long, may be saturated or unsaturated, and may be attached to functional groups containing oxygen, halogens, nitrogen, and sulfur. If a fatty acid contains a double bond, there is the possibility of either a cis or trans geometric isomerism, which significantly affects the molecule's configuration. Cis-double bonds cause the fatty acid chain to bend, an effect that is compounded with more double bonds in the chain. Other major lipid classes in the fatty acid category are the fatty esters and fatty amides.

Glycerolipids are composed of mono-, di-, and tri-substituted glycerols, the best-known being the fatty acid triesters of glycerol, called triglycerides. The word “triacylglycerol” is sometimes used synonymously with “triglyceride”. In these compounds, the three hydroxyl groups of glycerol are each esterified, typically by different fatty acids. Additional subclasses of glycerolipids are represented by glycosylglycerols, which are characterized by the presence of one or more sugar residues attached to glycerol via a glycosidic linkage.

Glycerophospholipids are amphipathic molecules (containing both hydrophobic and hydrophilic regions) that contain a glycerol core linked to two fatty acid-derived “tails” by ester linkages and to one “head” group by a phosphate ester linkage. Examples of glycerophospholipids, usually referred to as phospholipids (though sphingomyelins are also classified as phospholipids) are phosphatidylcholine (also known as PC, GPCho or lecithin), phosphatidylethanolamine (PE or GPEtn) and phosphatidylserine (PS or GPSer).

Sphingolipids are members of a complex family of compounds that share a common structural feature, a sphingoid base backbone. The major sphingoid base in mammals is commonly referred to as sphingosine. Ceramides (N-acyl-sphingoid bases) are a major subclass of sphingoid base derivatives with an amide-linked fatty acid. The fatty acids are typically saturated or mono-unsaturated with chain lengths from 16 to 26 carbon atoms. The major phosphosphingolipids of mammals are sphingomyelins (ceramide phosphocholines), whereas insects contain mainly ceramide phosphoethanolamines and fungi have phytoceramide phosphoinositols and mannose-containing headgroups. The glycosphingolipids are a diverse family of molecules composed of one or more sugar residues linked via a glycosidic bond to the sphingoid base. Examples of these are the simple and complex glycosphingolipids such as cerebrosides and gangliosides.

Sterols, such as cholesterol and its derivatives, or tocopherol and its derivatives, are important components of membrane lipids, along with the glycerophospholipids and sphingomyelins.

Saccharolipids are compounds in which fatty acids are linked directly to a sugar backbone, forming structures that are compatible with membrane bilayers. In the saccharolipids, a monosaccharide substitutes for the glycerol backbone present in glycerolipids and glycerophospholipids. The most familiar saccharolipids are the acylated glucosamine precursors of the Lipid A component of the lipopolysaccharides in Gram-negative bacteria. Typical lipid A molecules are disaccharides of glucosamine, which are derivatized with as many as seven fatty-acyl chains. The minimal lipopolysaccharide required for growth in E. coli is Kdo2-Lipid A, a hexa-acylated disaccharide of glucosamine that is glycosylated with two 3-deoxy-D-manno-octulosonic acid (Kdo) residues.

Polyketides are synthesized by polymerization of acetyl and propionyl subunits by classic enzymes as well as iterative and multimodular enzymes that share mechanistic features with the fatty acid synthases. They comprise a large number of secondary metabolites and natural products from animal, plant, bacterial, fungal and marine sources, and have great structural diversity. Many polyketides are cyclic molecules whose backbones are often further modified by glycosylation, methylation, hydroxylation, oxidation, or other processes.

Lipids and lipid-like materials may be cationic, anionic or neutral. Neutral lipids or lipid-like materials exist in an uncharged or neutral zwitterionic form at a selected pH.

In some embodiments, suitable lipids or lipid-like materials for use in the present disclosure include those described in WO2020/128031 and US20200163878, the entire contents of each of which are incorporated herein by reference for the purposes described herein.

### 2. Cationic or Cationically Ionizable Lipids or Lipid-Like Materials

In some embodiments cationic or cationically ionizable lipids or lipid-like materials contemplated for use herein include any cationic or cationically ionizable lipids or lipid-like materials which are able to electrostatically bind nucleic acid. In one embodiment, cationic or cationically ionizable lipids or lipid-like materials contemplated for use herein can be associated with nucleic acid, e.g. by forming complexes with the nucleic acid or forming vesicles in which the nucleic acid is enclosed or encapsulated.

Cationic lipids or lipid-like materials are characterized in that they have a net positive charge (e.g., at a relevant pH). Cationic lipids or lipid-like materials bind negatively charged nucleic acid by electrostatic interaction. Generally, cationic lipids possess a lipophilic moiety, such as a sterol, an acyl chain, a diacyl or more acyl chains, and the head group of the lipid typically carries the positive charge.

In certain embodiments, a cationic lipid or lipid-like material has a net positive charge only at certain pH, in particular acidic pH, while it has preferably no net positive charge, preferably has no charge, i.e., it is neutral, at a different, preferably higher pH such as physiological pH. This ionizable behavior is thought to enhance efficacy through helping with endosomal escape and reducing toxicity as compared with particles that remain cationic at physiological pH.

In some embodiments, a cationic or cationically ionizable lipid or lipid-like material comprises a head group which includes at least one nitrogen atom (N) which is positive charged or capable of being protonated.

Examples of cationic lipids include, but are not limited to 1,2-dioleoyl-3-trimethylammonium propane (DOTAP); N,N-dimethyl-2,3-dioleyloxypropylamine (DODMA), 1,2-di-O-octadecenyl-3-trimethylammonium propane (DOTMA), 3-(N—(N′,N′-dimethylaminoethane)-carbamoyl) cholesterol (DC-Chol), dimethyldioctadecylammonium (DDAB); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-distearyloxy-N,N-dimethyl-3-aminopropane (DSDMA), 2,3-di(tetradecoxy) propyl-(2-hydroxyethyl)-dimethylazanium (DMRIE), 1,2-dimyristoyl-sn-glycero-3-ethylphosphocholine (DMEPC), 1,2-dimyristoyl-3-trimethylammonium propane (DMTAP), 1,2-dioleyloxypropyl-3-dimethyl-hydroxyethyl ammonium bromide (DORIE), and 2,3-dioleoyloxy-N-[2 (spermine carboxamide)ethyl]-N,N-dimethyl-I-propanamium trifluoroacetate (DOSPA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-1-(cis,cis-9,12-oc-tadecadienoxy) propane (CLinDMA), 2-[5′-(cholest-5-en-3-beta-oxy)-3′-oxapentoxy)-3-dimethyl-1-(cis,cis-9′,12′-octadecadienoxy) propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino) butanoate (DLin-MC3-DMA), N-(2-Hydroxyethyl)-N,N-dimethyl-2,3-bis(tetradecyloxy)-1-propanaminium bromide (DMRIE), (±)-N-(3-aminopropyl)-N,N-dimethyl-2,3-bis(cis-9-tetradecenyloxy)-1-propanaminium bromide (GAP-DMORIE), (±)-N-(3-aminopropyl)-N,N-dimethyl-2,3-bis(dodecyloxy)-1-propanaminium bromide (GAP-DLRIE), (±)-N-(3-aminopropyl)-N,N-dimethyl-2,3-bis(tetradecyloxy)-1-propanaminium bromide (GAP-DMRIE), N-(2-Aminoethyl)-N,N-dimethyl-2,3-bis(tetradecyloxy)-1-propanaminium bromide (BAE-DMRIE), N-(4-carboxybenzyl)-N,N-dimethyl-2,3-bis(oleoyloxy) propan-1-aminium (DOBAQ), 2-({8-[(3 (3)-cholest-5-en-3-yloxy]octyl}oxy)-N,N-dimethyl-3-[(9Z,12Z)-octadeca-9,12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), N₁-[2-((1S)-1-[(3-aminopropyl)amino]-4-[di(3-amino-propyl)amino]butylcarboxamido)ethyl]-3,4-di [oleyloxy]-benzamide (MVL5), 1,2-dioleoyl-sn-glycero-3-ethylphosphocholine (DOEPC), 2,3-bis(dodecyloxy)-N-(2-hydroxyethyl)-N,N-dimethylpropan-1-amonium bromide (DLRIE), N-(2-aminoethyl)-N,N-dimethyl-2,3-bis(tetradecyloxy) propan-1-aminium bromide (DMORIE), di((Z)-non-2-en-1-yl) 8,8′-((((2 (dimethylamino)ethyl)thio) carbonyl) azanediyl)dioctanoate (ATX), N,N-dimethyl-2,3-bis(dodecyloxy) propan-1-amine (DLDMA), N,N-dimethyl-2,3-bis(tetradecyloxy) propan-1-amine (DMDMA), Di((Z)-non-2-en-1-yl)-9-((4-(dimethylaminobutanoyl)oxy) heptadecanedioate (L319), N-Dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethylamino) propionamide (lipidoid 98N₁₂-5), 1-[2-[bis(2-hydroxydodecyl)amino]ethyl-[2-[4-[2-[bis (2 hydroxydodecyl)amino]ethyl]piperazin-1-yl]ethyl]amino]dodecan-2-ol (lipidoid C12-200), LIPOFECTIN® (commercially available cationic liposomes comprising DOTMA and 1,2-dioleoyl-sn-3phosphoethanolamine (DOPE), from GIBCO/BRL, Grand Island, N.Y.); LIPOFECTAMINE® (commercially available cationic liposomes comprising N-(1-(2,3dioleyloxy) propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.) or any combination of any of the foregoing. Further suitable cationic lipids for use in the present disclosure include those described in WO2020/128031 and US20200163878, the entire contents of each of which are incorporated herein by reference for the purposes described herein. Further suitable cationic lipids for use in the present disclosure include those described in WO2010/053572 (including CI 2-200 described at paragraph) [00225]), and WO2012/170930, both of which are incorporated herein by reference for the purposes described herein. Additional suitable cationic lipids for use in the present disclosure include HGT4003, HGT5000, HGTS001, HGT5001, HGT5002 (see US20150140070A1, which is incorporated herein by reference in its entirety).

In some embodiments, formulations that are useful for pharmaceutical compositions (e.g., immunogenic compositions, e.g., vaccines) compositions as described herein can comprise at least one cationic lipid. Representative cationic lipids include, but are not limited to, 1,2-dilinoleyoxy-3-(dimethylamino) acetoxypropane (DLin-DAC), 1,2-dilinoleyoxy-3morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3dimethylaminopropane (DLin-2-DMAP), 1,2-dilinoleyloxy-3-trimethylaminopropane chloride salt (DLin-TMA.CI), 1,2-dilinoleoyl-3-trimethylaminopropane chloride salt (DLin-TAP.CI), 1,2-dilinoleyloxy-3-(N-methylpiperazino) propane (DLin-MPZ), 3-(N,Ndilinoleylamino)-1,2-propanediol (DLinAP), 3-(N,N-dioleylamino)-1, 2-propanediol (DOAP), 1,2-dilinoleyloxo-3-(2-N,N-dimethylamino) ethoxypropane (DLin-EG-DMA), and 2,2-dilinoleyl-4-dimethylaminomethyl-[1,3]-dioxolane (DLin-K-DMA), 2,2-dilinoleyl-4-(2-dimethylaminoethyl)-[1,3]-dioxolane (DLin-KC2-DMA); dilinoleyl-methyl-4-dimethylaminobutyrate (DLin-MC3-DMA); MC3 (US20100324120, which is incorporated herein by reference in its entirety).

In some embodiments, amino or cationic lipids useful in accordance with the present disclosure have at least one protonatable or deprotonatable group, such that the lipid is positively charged at a pH at or below physiological pH (e.g. pH 7.4), and neutral at a second pH, preferably at or above physiological pH. It will, of course, be understood that the addition or removal of protons as a function of pH is an equilibrium process, and that the reference to a charged or a neutral lipid refers to the nature of the predominant species and does not require that all of lipids have to be present in the charged or neutral form. Lipids having more than one protonatable or deprotonatable group, or which are zwitterionic, are not excluded and may likewise suitable in the context of the present invention.

In some embodiments, a protonatable lipid has a pKa of the protonatable group in the range of about 4 to about 11, e.g., a pKa of about 5 to about 7.

In some embodiments, a cationic lipid may comprise from about 10 mol % to about 100 mol %, about 20 mol % to about 100 mol %, about 30 mol % to about 100 mol %, about 40 mol % to about 100 mol %, or about 50 mol % to about 100 mol % of total lipid present in a lipid composition utilized in accordance with the present disclosure.

### 3. Additional Lipids or Lipid-Like Materials

In some embodiments, formulations utilized in accordance with the present disclosure may comprise lipids or lipid-like materials other than cationic or cationically ionizable lipids or lipid-like materials, i.e., non-cationic lipids or lipid-like materials (including non-cationically ionizable lipids or lipid-like materials). Collectively, anionic and neutral lipids or lipid-like materials are referred to herein as non-cationic lipids or lipid-like materials. In some embodiments, optimizing a formulation of nucleic acid particles by addition of other hydrophobic moieties, such as cholesterol and lipids, in addition to an ionizable/cationic lipid or lipid-like material may, for example, enhance particle stability and efficacy of nucleic acid delivery.

In some embodiments, a lipid or lipid-like material may be incorporated which may or may not affect the overall charge of particles. In certain embodiments, such lipid or lipid-like material is a non-cationic lipid or lipid-like material.

In some embodiments, a non-cationic lipid may comprise, e.g., one or more anionic lipids and/or neutral lipids. An “anionic lipid” is negatively charged (e.g., at a selected pH).

A “neutral lipid” exists either in an uncharged or neutral zwitterionic form (e.g., at a selected pH). In some embodiments, a formulation comprises one of the following neutral lipid components: (1) a phospholipid, (2) cholesterol or a derivative thereof; or (3) a mixture of a phospholipid and cholesterol or a derivative thereof. Examples of cholesterol derivatives include, but are not limited to, cholestanol, cholestanone, cholestenone, coprostanol, cholesteryl-2′-hydroxyethyl ether, cholesteryl-4′-hydroxybutyl ether, tocopherol and derivatives thereof, and mixtures thereof.

Specific exemplary phospholipids that can be used include, but are not limited to, phosphatidylcholines, phosphatidylethanolamines, phosphatidylglycerols, phosphatidic acids, phosphatidylserines or sphingomyelin. Such phospholipids include in particular diacylphosphatidylcholines, such as distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC) and phosphatidylethanolamines, in particular diacylphosphatidylethanolamines, such as dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), diphytanoyl-phosphatidylethanolamine (DPyPE), and further phosphatidylethanolamine lipids with different hydrophobic chains.

In certain embodiments, a formulation utilized in accordance with the present disclosure includes DSPC or DSPC and cholesterol.

In certain embodiments, formulations utilized in accordance with the present disclosure include both a cationic lipid and an additional (non-cationic) lipid.

In some embodiments, formulations herein include a polymer conjugated lipid such as a pegylated lipid. “Pegylated lipids” comprise both a lipid portion and a polyethylene glycol portion. Pegylated lipids are known in the art.

Without wishing to be bound by theory, the amount of (total) cationic lipid compared to the amount of other lipid(s) in formulation may affect important characteristics, such as charge, particle size, stability, tissue selectivity, and bioactivity of the nucleic acid. In some embodiments, the molar ratio of the at least one cationic lipid to the at least one additional lipid is from about 10:0 to about 1:9, about 4:1 to about 1:2, or about 3:1 to about 1:1.

In some embodiments, a non-cationic lipid, in particular a neutral lipid, (e.g., one or more phospholipids and/or cholesterol) may comprise from about 0 mol % to about 90 mol %, from about 0 mol % to about 80 mol %, from about 0 mol % to about 70 mol %, from about 0 mol % to about 60 mol %, or from about 0 mol % to about 50 mol %, of the total lipid present in a formulation.

### 4. Lipoplex Particles

In certain embodiments of the present disclosure, the RNA described herein may be present in RNA lipoplex particles.

An “RNA lipoplex particle” contains lipid, in particular cationic lipid, and RNA. Electrostatic interactions between positively charged liposomes and negatively charged RNA results in complexation and spontaneous formation of RNA lipoplex particles. Positively charged liposomes may be generally synthesized using a cationic lipid, such as DOTMA, and additional lipids, such as DOPE. In one embodiment, a RNA lipoplex particle is a nanoparticle.

In certain embodiments, RNA lipoplex particles include both a cationic lipid and an additional lipid. In an exemplary embodiment, the cationic lipid is DOTMA and the additional lipid is DOPE.

In some embodiments, the molar ratio of the at least one cationic lipid to the at least one additional lipid is from about 10:0 to about 1:9, about 4:1 to about 1:2, or about 3:1 to about 1:1. In specific embodiments, the molar ratio may be about 3:1, about 2.75:1, about 2.5:1, about 2.25:1, about 2:1, about 1.75:1, about 1.5:1, about 1.25:1, or about 1:1. In an exemplary embodiment, the molar ratio of the at least one cationic lipid to the at least one additional lipid is about 2:1.

In some embodiments, RNA lipoplex particles have an average diameter that in one embodiment ranges from about 200 nm to about 1000 nm, from about 200 nm to about 800 nm, from about 250 to about 700 nm, from about 400 to about 600 nm, from about 300 nm to about 500 nm, or from about 350 nm to about 400 nm. In specific embodiments, the RNA lipoplex particles have an average diameter of about 200 nm, about 225 nm, about 250 nm, about 275 nm, about 300 nm, about 325 nm, about 350 nm, about 375 nm, about 400 nm, about 425 nm, about 450 nm, about 475 nm, about 500 nm, about 525 nm, about 550 nm, about 575 nm, about 600 nm, about 625 nm, about 650 nm, about 700 nm, about 725 nm, about 750 nm, about 775 nm, about 800 nm, about 825 nm, about 850 nm, about 875 nm, about 900 nm, about 925 nm, about 950 nm, about 975 nm, or about 1000 nm. In an embodiment, the RNA lipoplex particles have an average diameter that ranges from about 250 nm to about 700 nm. In another embodiment, the RNA lipoplex particles have an average diameter that ranges from about 300 nm to about 500 nm. In an exemplary embodiment, the RNA lipoplex particles have an average diameter of about 400 nm.

RNA lipoplex particles and compositions comprising RNA lipoplex particles described herein are useful for delivery of RNA to a target tissue after parenteral administration, in particular after intravenous administration. The RNA lipoplex particles may be prepared using liposomes that may be obtained by injecting a solution of the lipids in ethanol into water or a suitable aqueous phase. In one embodiment, the aqueous phase has an acidic pH. In one embodiment, the aqueous phase comprises acetic acid, e.g., in an amount of about 5 mM. Liposomes may be used for preparing RNA lipoplex particles by mixing the liposomes with RNA. In one embodiment, the liposomes and RNA lipoplex particles comprise at least one cationic lipid and at least one additional lipid. In one embodiment, the at least one cationic lipid comprises 1,2-di-O-octadecenyl-3-trimethylammonium propane (DOTMA) and/or 1,2-dioleoyl-3-trimethylammonium-propane (DOTAP). In one embodiment, the at least one additional lipid comprises 1,2-di-(9Z-octadecenoyl)-sn-glycero-3-phosphoethanolamine (DOPE), cholesterol (Chol) and/or 1,2-dioleoyl-sn-glycero-3-phosphocholine (DOPC). In one embodiment, the at least one cationic lipid comprises 1,2-di-O-octadecenyl-3-trimethylammonium propane (DOTMA) and the at least one additional lipid comprises 1,2-di-(9Z-octadecenoyl)-sn-glycero-3-phosphoethanolamine (DOPE). In one embodiment, the liposomes and RNA lipoplex particles comprise 1,2-di-O-octadecenyl-3-trimethylammonium propane (DOTMA) and 1,2-di-(9Z-octadecenoyl)-sn-glycero-3-phosphoethanolamine (DOPE).

Spleen targeting RNA lipoplex particles are described in WO 2013/143683, herein incorporated by reference. It has been found that RNA lipoplex particles having a net negative charge may be used to preferentially target spleen tissue or spleen cells such as antigen-presenting cells, in particular dendritic cells. Accordingly, following administration of the RNA lipoplex particles, RNA accumulation and/or RNA expression in the spleen occurs. Thus, RNA lipoplex particles of the disclosure may be used for expressing RNA in the spleen. In an embodiment, after administration of the RNA lipoplex particles, no or essentially no RNA accumulation and/or RNA expression in the lung and/or liver occurs. In one embodiment, after administration of the RNA lipoplex particles, RNA accumulation and/or RNA expression in antigen presenting cells, such as professional antigen presenting cells in the spleen occurs. Thus, RNA lipoplex particles of the disclosure may be used for expressing RNA in such antigen presenting cells. In one embodiment, the antigen presenting cells are dendritic cells and/or macrophages.

### 5. Lipid Nanoparticles (LNPs)

In some embodiments, nucleic acid such as RNA described herein is administered in the form of lipid nanoparticles (LNPs). In some embodiments, LNPs may comprise any lipid capable of forming a particle to which the one or more nucleic acid molecules are attached, or in which the one or more nucleic acid molecules are encapsulated.

In some embodiments, an LNP comprises one or more cationic lipids, and one or more stabilizing lipids. Stabilizing lipids include neutral lipids and pegylated lipids.

In some embodiments, an LNP comprises a cationic lipid, a neutral lipid, a sterol, a polymer conjugated lipid; and an RNA, encapsulated within or associated with the lipid nanoparticle.

In some embodiments, a neutral lipid is selected from the group consisting of DSPC, DPPC, DMPC, DOPC, POPC, DOPE, DOPG, DPPG, POPE, DPPE, DMPE, DSPE, and SM. In some embodiments, the neutral lipid is selected from the group consisting of DSPC, DPPC, DMPC, DOPC, POPC, DOPE and SM. In some embodiments, the neutral lipid is DSPC.

In some embodiments, a sterol is cholesterol.

In some embodiments, a polymer conjugated lipid is a pegylated lipid. In some embodiments, a pegylated lipid has the following structure:

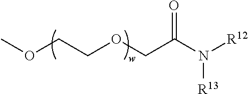

or a pharmaceutically acceptable salt, tautomer or stereoisomer thereof, wherein: R¹² and R¹³ are each independently a straight or branched, saturated or unsaturated alkyl chain containing from 10 to 30 carbon atoms, wherein the alkyl chain is optionally interrupted by one or more ester bonds; and w has a mean value ranging from 30 to 60. In some embodiments, R¹² and R¹³ are each independently straight, saturated alkyl chains containing from 12 to 16 carbon atoms. In some embodiments, w has a mean value ranging from 40 to 55. In some embodiments, the average w is about 45. In some embodiments, R¹² and R¹³ are each independently a straight, saturated alkyl chain containing about 14 carbon atoms, and w has a mean value of about 45.

In some embodiments, a pegylated lipid is DMG-PEG 2000, e.g., having the following structure:

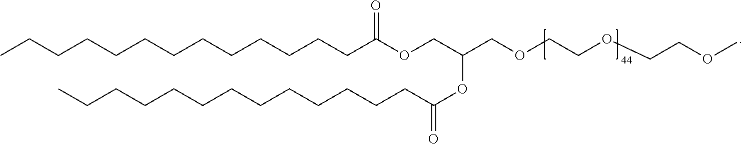

In some embodiments, a cationic lipid component of LNPs has the structure of Formula (III):

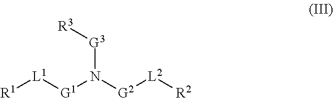

or a pharmaceutically acceptable salt, tautomer, prodrug or stereoisomer thereof, wherein:
one of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O) NR^{a}—, NR^{a}C(═O) NR^{a}—, —OC(═O) NR^{a}- or —NR^{a}C(═O)O—, and the other of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O) NR^{a}—, NR^{a}C(═O) NR^{a}—, —OC(═O) NR^{a}- or —NR^{a}C(═O)O- or a direct bond;
G₁ and G₂ are each independently unsubstituted C₁-C₁₂ alkylene or C1-C₁₂ alkenylene;
G₃ is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR5, CN, —C(═O) OR4, —OC(═O)R⁴ or —NR⁵C(═O) R⁴;
R⁴ is C₁-C₁₂ alkyl;
R⁵ is H or C₁-C₆ alkyl; and
x is 0, 1 or 2.

In some of the foregoing embodiments of Formula (III), the lipid has one of the following structures (IIIA) or (IIIB):

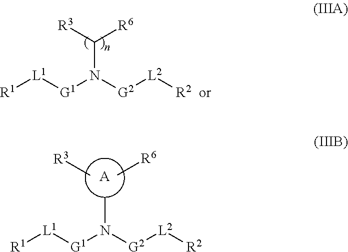

wherein:
A is a 3 to 8-membered cycloalkyl or cycloalkylene ring;
R⁶ is, at each occurrence, independently H, OH or C₁-C₂₄ alkyl;
n is an integer ranging from 1 to 15.

In some of the foregoing embodiments of Formula (III), the lipid has structure (IIIA), and in other embodiments, the lipid has structure (IIIB).

In other embodiments of Formula (III), the lipid has one of the following structures (IIIC) or (IIID):

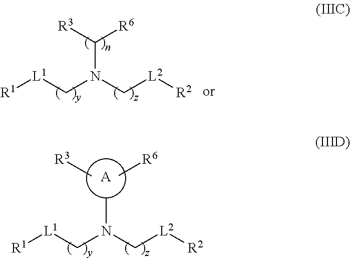

wherein y and z are each independently integers ranging from 1 to 12.

In any of the foregoing embodiments of Formula (III), one of L¹ or L² is —O(C═O)—. For example, in some embodiments each of L¹ and L² are —O(C═O)—. In some different embodiments of any of the foregoing, L¹ and L² are each independently —(C═O)O— or —O(C═O)—. For example, in some embodiments each of L¹ and L² is —(C═O)O—.

In some different embodiments of Formula (III), the lipid has one of the following structures (IIIE) or (IIIF):

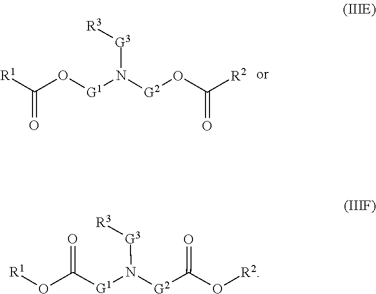

In some of the foregoing embodiments of Formula (III), the lipid has one of the following structures (IIIG), (IIIH), (IIII), or (IIIJ):

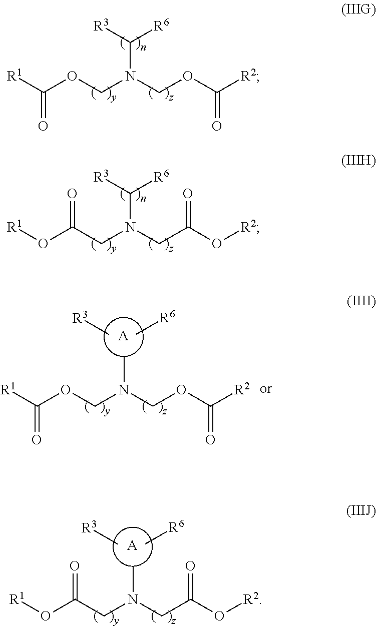

In some of the foregoing embodiments of Formula (III), n is an integer ranging from 2 to 12, for example from 2 to 8 or from 2 to 4. For example, in some embodiments, n is 3, 4, 5 or 6. In some embodiments, n is 3. In some embodiments, n is 4. In some embodiments, n is 5. In some embodiments, n is 6.

In some other of the foregoing embodiments of Formula (III), y and z are each independently an integer ranging from 2 to 10. For example, in some embodiments, y and z are each independently an integer ranging from 4 to 9 or from 4 to 6.

In some of the foregoing embodiments of Formula (III), R⁶ is H. In other of the foregoing embodiments, R⁶ is C₁-C₂₄ alkyl. In other embodiments, R⁶ is OH.

In some embodiments of Formula (III), G₃ is unsubstituted. In other embodiments, G₃ is substituted. In various different embodiments, G₃ is linear C₁-C₂₄ alkylene or linear C₁-C₂₄ alkenylene.

In some other foregoing embodiments of Formula (III), R¹ or R², or both, is C₆-C₂₄ alkenyl. For example, in some embodiments, R¹ and R² each, independently have the following structure:

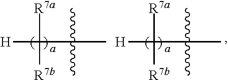

wherein:
R^{7a} and R^{7b} are, at each occurrence, independently H or C₁-C₁₂ alkyl; and
a is an integer from 2 to 12,
wherein R^{7a}, R^{7b} and a are each selected such that R¹ and R² each independently comprise from 6 to 20 carbon atoms. For example, in some embodiments a is an integer ranging from 5 to 9 or from 8 to 12.

In some of the foregoing embodiments of Formula (III), at least one occurrence of R^{7a} is H. For example, in some embodiments, R^{7a} is H at each occurrence. In other different embodiments of the foregoing, at least one occurrence of R^{7b} is C₁-C₈alkyl. For example, in some embodiments, C₁-C₈alkyl is methyl, ethyl, n-propyl, iso-propyl, n-butyl, iso-butyl, tert-butyl, n-hexyl or n-octyl.

In different embodiments of Formula (III), R¹ or R², or both, has one of the following structures:

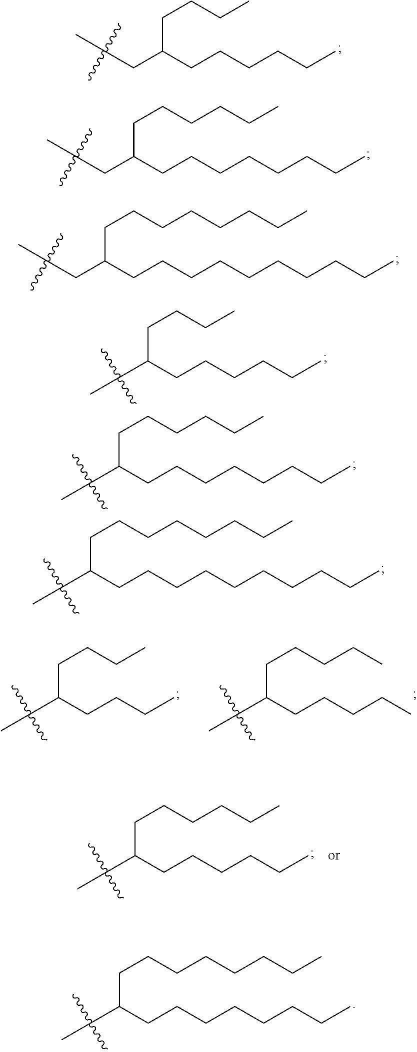

In some of the foregoing embodiments of Formula (III), R³ is OH, CN, —C(═O) OR4, —OC(═O) R⁴ or —NHC(═O) R⁴. In some embodiments, R⁴ is methyl or ethyl.

In various different embodiments, the cationic lipid of Formula (III) has one of the structures set forth in in Table 8 below.

TABLE 8
Exemplary Compounds of Formula (III)

| No. | Structure |
|---|---|
| III-1 | 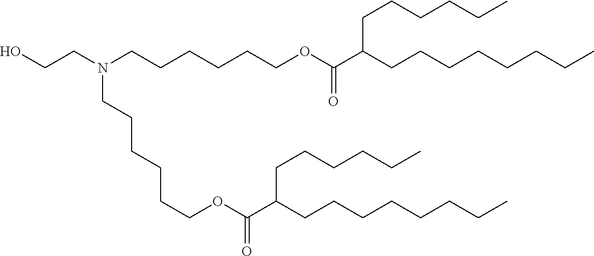 [SMILES: CCCCCCCCC(CCCCCC)C(=O)OCCCCCCN(CCO)CCCCCCOC(=O)C(CCCCCC)CCCCCCCC] |
| III-2 | 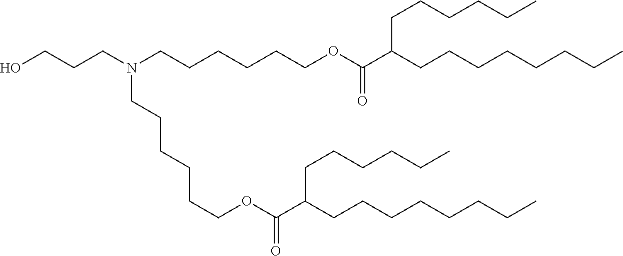 [SMILES: CCCCCCCCC(CCCCCC)C(=O)OCCCCCCN(CCCO)CCCCCCOC(=O)C(CCCCCC)CCCCCCCC] |
| III-3 | 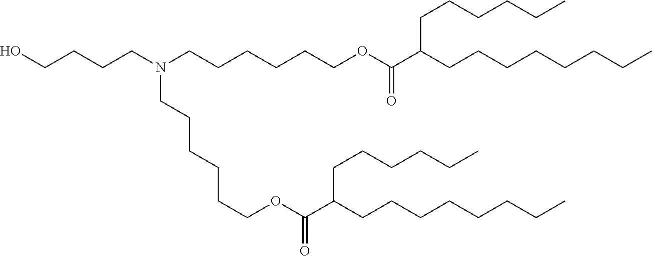 [SMILES: CCCCCCCCC(CCCCCC)C(=O)OCCCCCCN(CCCCO)CCCCCCOC(=O)C(CCCCCC)CCCCCCCC] |
| III-4 | 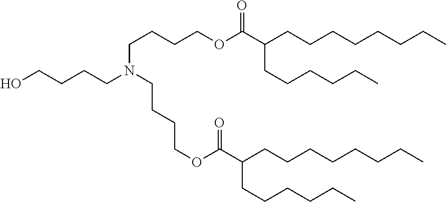 [SMILES: CCCCCCCCC(CCCCCC)C(=O)OCCCCN(CCCCO)CCCCOC(=O)C(CCCCCC)CCCCCCCC] |
| III-5 | 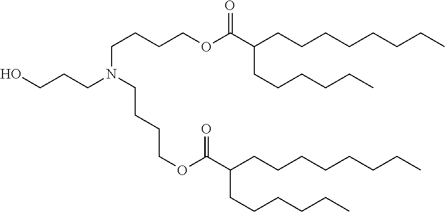 [SMILES: CCCCCCCCC(CCCCCC)C(=O)OCCCCN(CCCO)CCCCOC(=O)C(CCCCCC)CCCCCCCC] |
| III-6 | 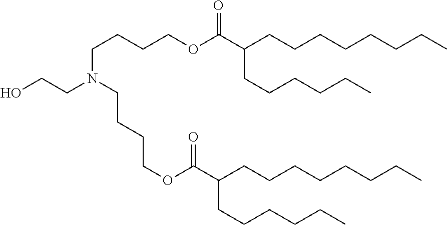 [SMILES: CCCCCCCCC(CCCCCC)C(=O)OCCCCN(CCO)CCCCOC(=O)C(CCCCCC)CCCCCCCC] |
| III-7 | 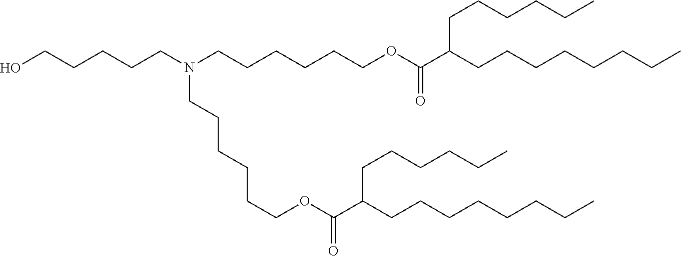 [SMILES: CCCCCCCCC(CCCCCC)C(=O)OCCCCCCN(CCCCCO)CCCCCCOC(=O)C(CCCCCC)CCCCCCCC] |
| III-8 | 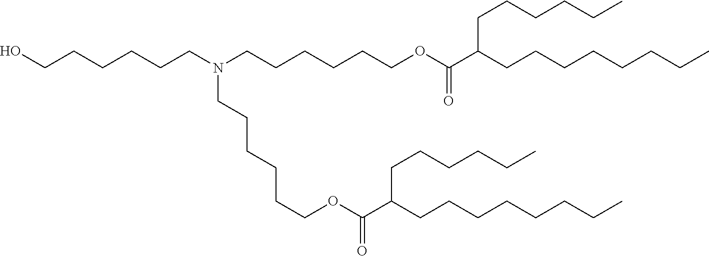 [SMILES: CCCCCCCCC(CCCCCC)C(=O)OCCCCCCN(CCCCCCO)CCCCCCOC(=O)C(CCCCCC)CCCCCCCC] |
| III-9 | 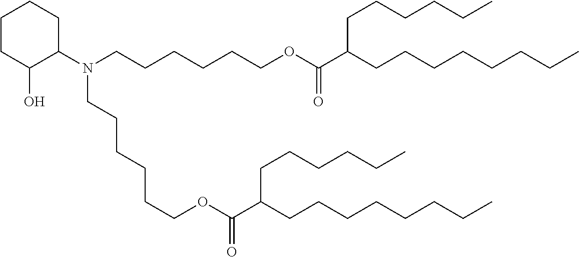 [SMILES: CCCCCCCCC(CCCCCC)C(=O)OCCCCCCN(CCCCCCOC(=O)C(CCCCCC)CCCCCCCC)C1CCCCC1O] |
| III-10 | 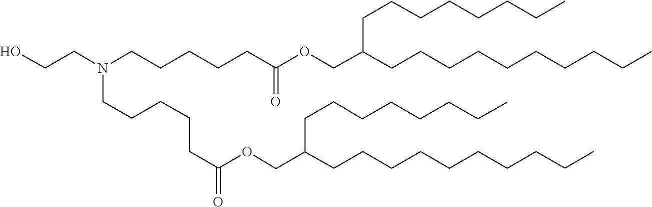 [SMILES: CCCCCCCCCCC(CCCCCCCC)COC(=O)CCCCCN(CCO)CCCCCC(=O)OCC(CCCCCCCC)CCCCCCCCCC] |
| | 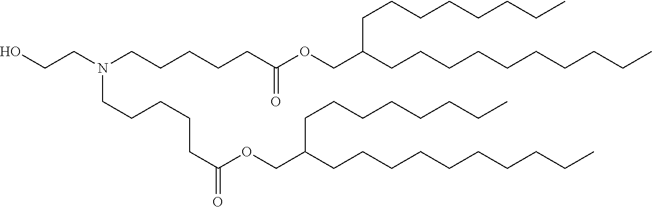 [SMILES: CCCCCCCCCCC(CCCCCCCC)COC(=O)CCCCCN(CCO)CCCCCC(=O)OCC(CCCCCCCC)CCCCCCCCCC] |
| III-11 | 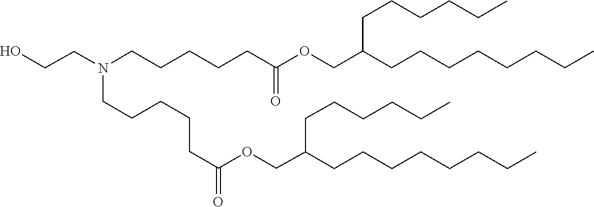 [SMILES: CCCCCCCCC(CCCCCC)COC(=O)CCCCCN(CCO)CCCCCC(=O)OCC(CCCCCC)CCCCCCCC] |
| | 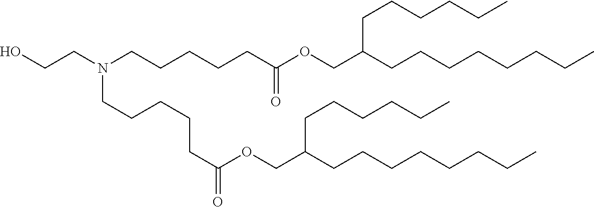 [SMILES: CCCCCCCCC(CCCCCC)COC(=O)CCCCCN(CCO)CCCCCC(=O)OCC(CCCCCC)CCCCCCCC] |
| III-12 | 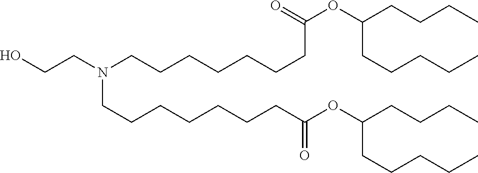 [SMILES: CCCCCC(CCCC)OC(=O)CCCCCCCN(CCO)CCCCCCCC(=O)OC(CCCC)CCCCC] |
| | 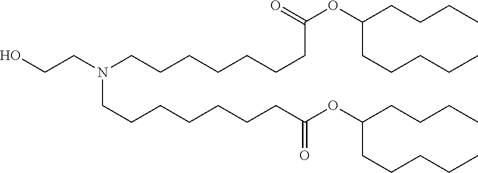 [SMILES: CCCCCC(CCCC)OC(=O)CCCCCCCN(CCO)CCCCCCCC(=O)OC(CCCC)CCCCC] |
| III-13 | 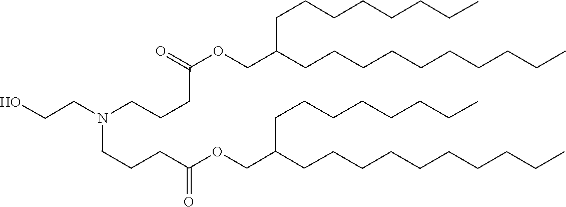 [SMILES: CCCCCCCCCCC(CCCCCCCC)COC(=O)CCCN(CCO)CCCC(=O)OCC(CCCCCCCC)CCCCCCCCCC] |
| | 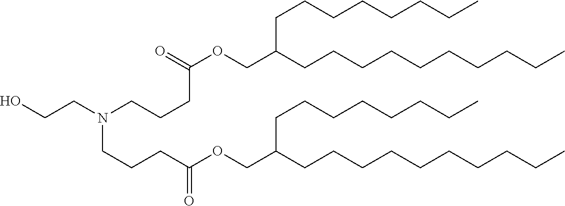 [SMILES: CCCCCCCCCCC(CCCCCCCC)COC(=O)CCCN(CCO)CCCC(=O)OCC(CCCCCCCC)CCCCCCCCCC] |
| III-14 | 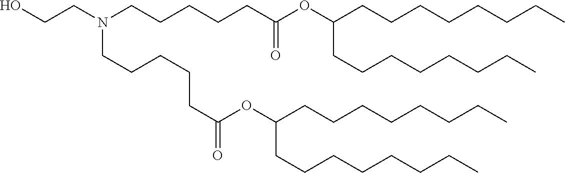 [SMILES: CCCCCCCCC(CCCCCCCC)OC(=O)CCCCCN(CCO)CCCCCC(=O)OC(CCCCCCCC)CCCCCCCC] |
| | 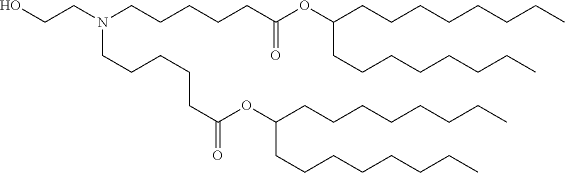 [SMILES: CCCCCCCCC(CCCCCCCC)OC(=O)CCCCCN(CCO)CCCCCC(=O)OC(CCCCCCCC)CCCCCCCC] |
| III-15 | 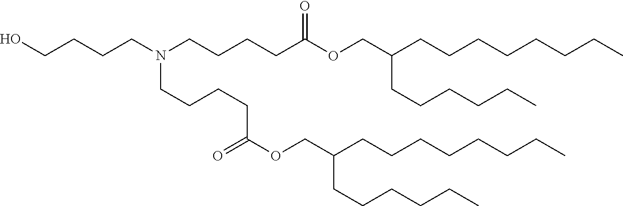 [SMILES: CCCCCCCCC(CCCCCC)COC(=O)CCCCN(CCCCO)CCCCC(=O)OCC(CCCCCC)CCCCCCCC] |
| | 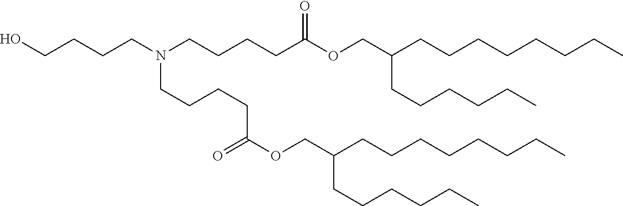 [SMILES: CCCCCCCCC(CCCCCC)COC(=O)CCCCN(CCCCO)CCCCC(=O)OCC(CCCCCC)CCCCCCCC] |
| III-16 | 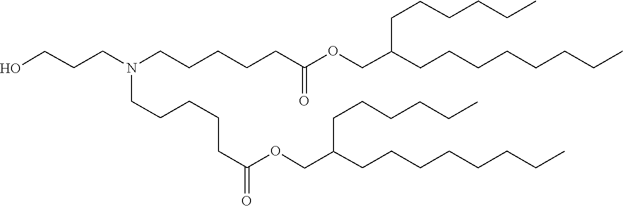 [SMILES: CCCCCCCCC(CCCCCC)COC(=O)CCCCCN(CCCO)CCCCCC(=O)OCC(CCCCCC)CCCCCCCC] |
| | 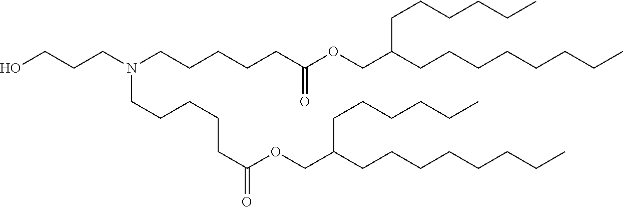 [SMILES: CCCCCCCCC(CCCCCC)COC(=O)CCCCCN(CCCO)CCCCCC(=O)OCC(CCCCCC)CCCCCCCC] |
| III-17 | 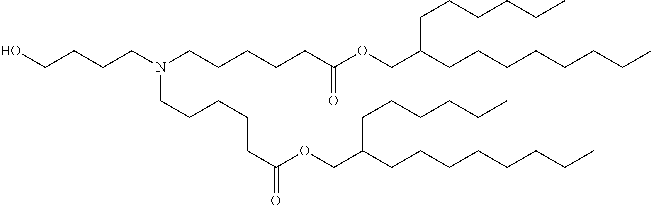 [SMILES: CCCCCCCCC(CCCCCC)COC(=O)CCCCCN(CCCCO)CCCCCC(=O)OCC(CCCCCC)CCCCCCCC] |
| | 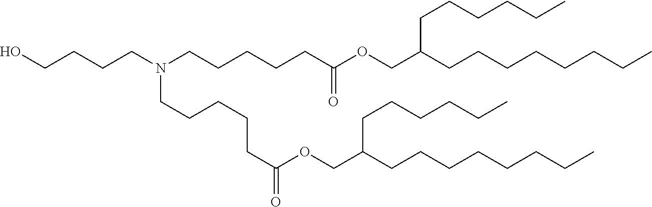 [SMILES: CCCCCCCCC(CCCCCC)COC(=O)CCCCCN(CCCCO)CCCCCC(=O)OCC(CCCCCC)CCCCCCCC] |
| III-18 | 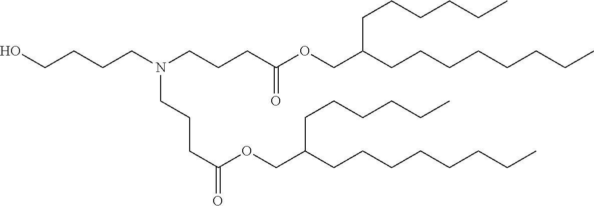 [SMILES: CCCCCCCCC(CCCCCC)COC(=O)CCCN(CCCCO)CCCC(=O)OCC(CCCCCC)CCCCCCCC] |
| III-19 | 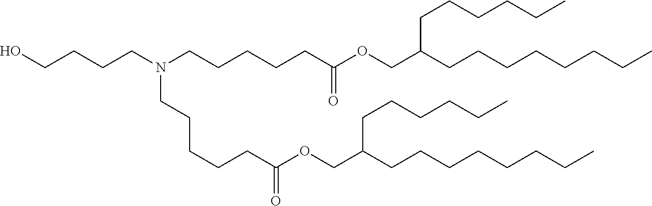 [SMILES: CCCCCCCCC(CCCCCC)COC(=O)CCCCCN(CCCCO)CCCCCC(=O)OCC(CCCCCC)CCCCCCCC] |
| III-20 | 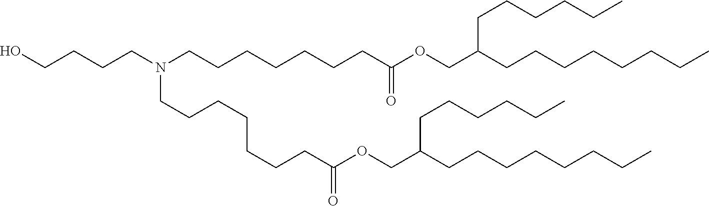 [SMILES: CCCCCCCCC(CCCCCC)COC(=O)CCCCCCCN(CCCCO)CCCCCCCC(=O)OCC(CCCCCC)CCCCCCCC] |
| III-21 | 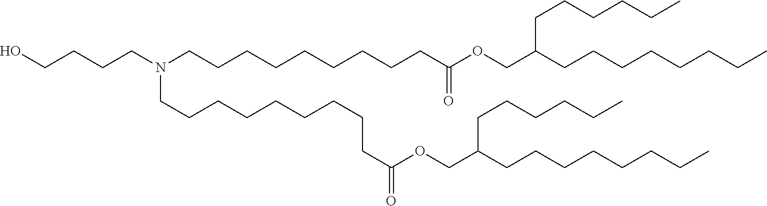 [SMILES: CCCCCCCCC(CCCCCC)COC(=O)CCCCCCCCCN(CCCCO)CCCCCCCCCC(=O)OCC(CCCCCC)CCCCCCCC] |
| III-22 | 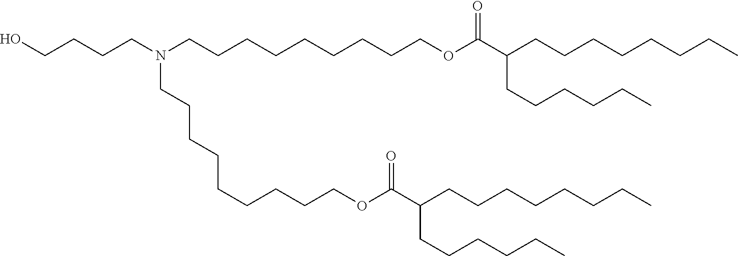 [SMILES: CCCCCCCCC(CCCCCC)C(=O)OCCCCCCCCCN(CCCCO)CCCCCCCCCOC(=O)C(CCCCCC)CCCCCCCC] |
| III-23 | 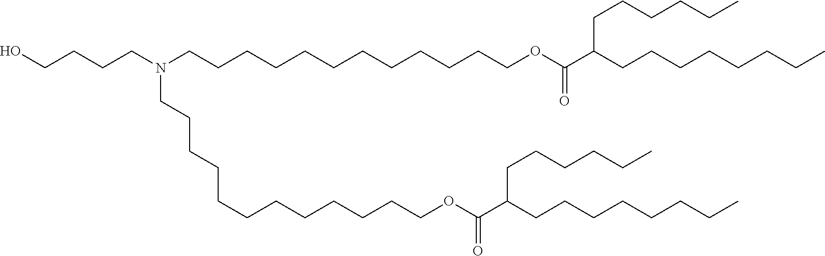 [SMILES: CCCCCCCCC(CCCCCC)C(=O)OCCCCCCCCCCCCN(CCCCO)CCCCCCCCCCCCOC(=O)C(CCCCCC)CCCCCCCC] |
| III-24 | 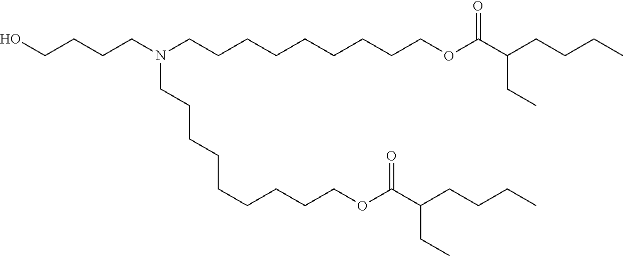 [SMILES: CCCCC(CC)C(=O)OCCCCCCCCCN(CCCCO)CCCCCCCCCOC(=O)C(CC)CCCC] |
| III-25 | 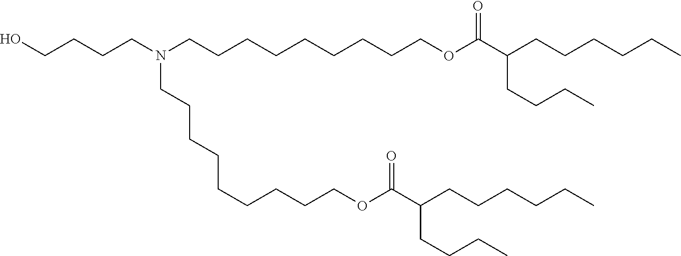 [SMILES: CCCCCCC(CCCC)C(=O)OCCCCCCCCCN(CCCCO)CCCCCCCCCOC(=O)C(CCCC)CCCCCC] |
| III-26 | 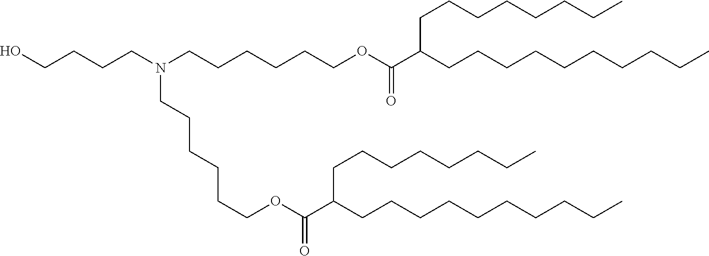 [SMILES: CCCCCCCCCCC(CCCCCCCC)C(=O)OCCCCCCN(CCCCO)CCCCCCOC(=O)C(CCCCCCCC)CCCCCCCCCC] |
| III-27 | 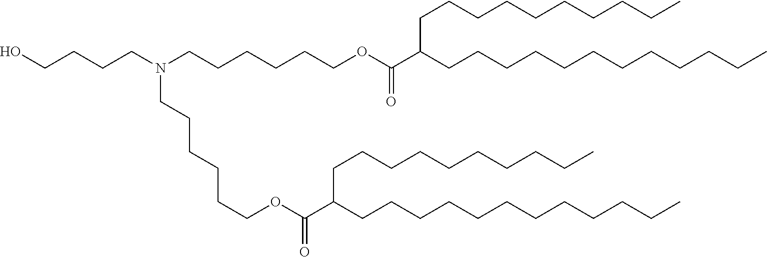 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] |
| III-28 | 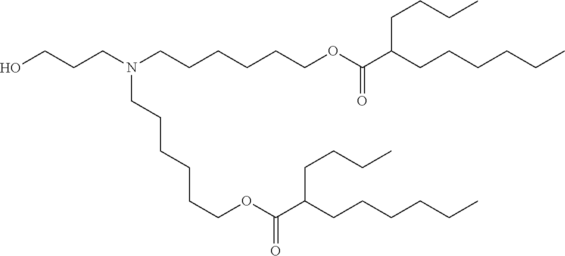 [SMILES: CCCCCCC(CCCC)C(=O)OCCCCCCN(CCCO)CCCCCCOC(=O)C(CCCC)CCCCCC] |
| III-29 | 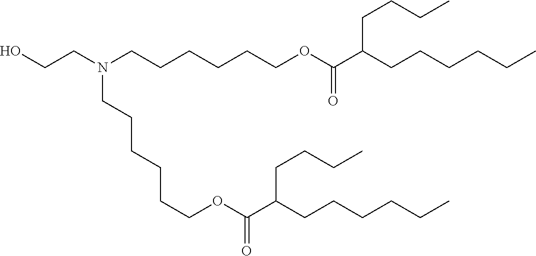 [SMILES: CCCCCCC(CCCC)C(=O)OCCCCCCN(CCO)CCCCCCOC(=O)C(CCCC)CCCCCC] |
| III-30 | 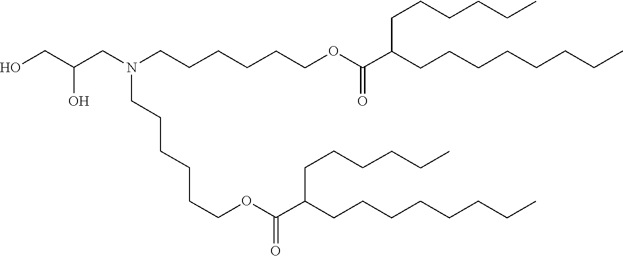 [SMILES: CCCCCCCCC(CCCCCC)C(=O)OCCCCCCN(CCCCCCOC(=O)C(CCCCCC)CCCCCCCC)CC(O)CO] |
| III-31 | 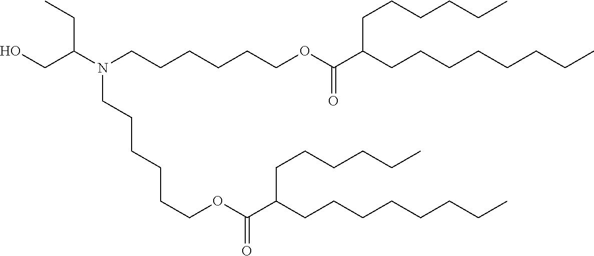 [SMILES: CCCCCCCCC(CCCCCC)C(=O)OCCCCCCN(CCCCCCOC(=O)C(CCCCCC)CCCCCCCC)C(CC)CO] |
| III-32 | 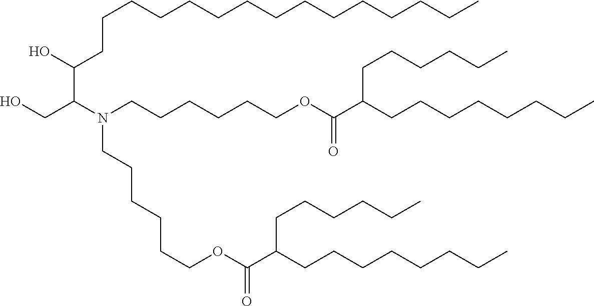 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] |
| III-33 | 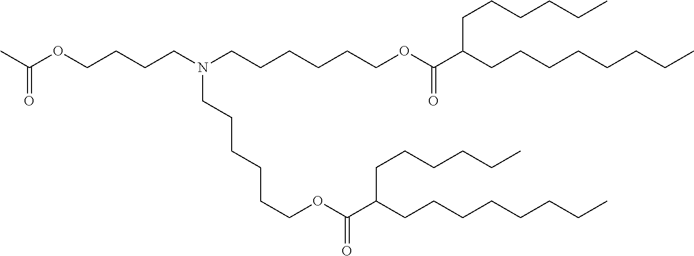 [SMILES: CCCCCCCCC(CCCCCC)C(=O)OCCCCCCN(CCCCCCOC(=O)C(CCCCCC)CCCCCCCC)CCCCOC(C)=O] |
| III-34 | 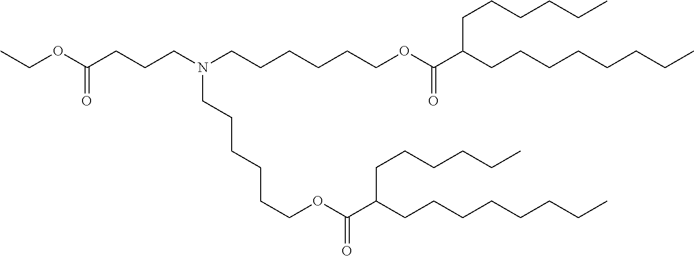 [SMILES: CCCCCCCCC(CCCCCC)C(=O)OCCCCCCN(CCCCCCOC(=O)C(CCCCCC)CCCCCCCC)CCCC(=O)OCC] |
| III-35 | 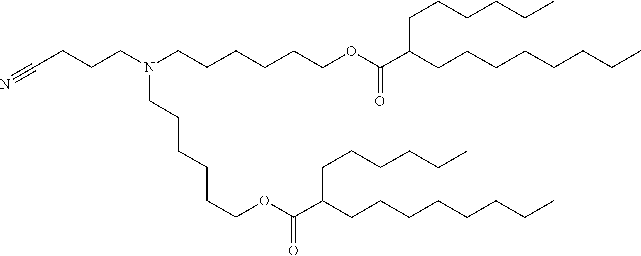 [SMILES: CCCCCCCCC(CCCCCC)C(=O)OCCCCCCN(CCCC#N)CCCCCCOC(=O)C(CCCCCC)CCCCCCCC] |
| III-36 | 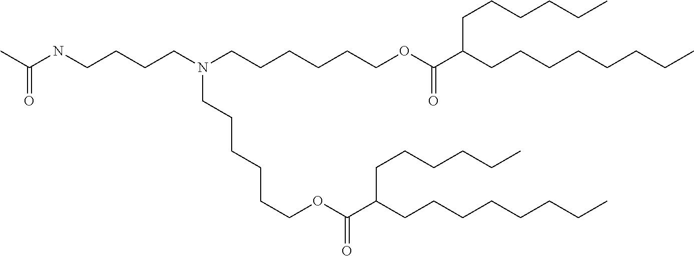 [SMILES: CCCCCCCCC(CCCCCC)C(=O)OCCCCCCN(CCCCCCOC(=O)C(CCCCCC)CCCCCCCC)CCCCNC(C)=O] |

In various different embodiments, a cationic lipid has one of the structures set forth in Table 9 below.

TABLE 9
Exemplary Cationic Lipid Structures

| No. | Structure |
|---|---|
| A | 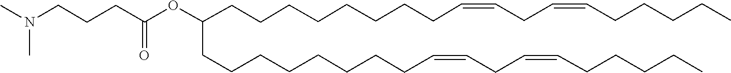 [SMILES: CCCCC/C=C\C/C=C\CCCCCCCCC(CCCCCCCC/C=C\C/C=C\CCCCC)OC(=O)CCCN(C)C] |
| | 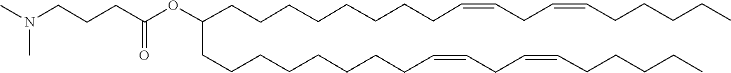 [SMILES: CCCCC/C=C\C/C=C\CCCCCCCCC(CCCCCCCC/C=C\C/C=C\CCCCC)OC(=O)CCCN(C)C] |
| B | 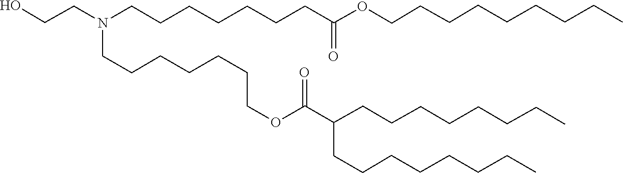 [SMILES: CCCCCCCCCOC(=O)CCCCCCCN(CCO)CCCCCCCOC(=O)C(CCCCCCCC)CCCCCCCC] |
| | 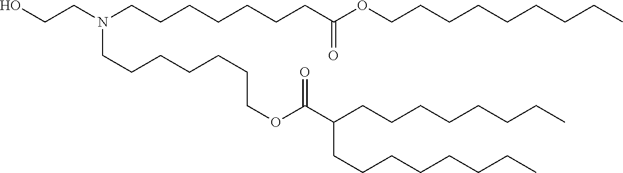 [SMILES: CCCCCCCCCOC(=O)CCCCCCCN(CCO)CCCCCCCOC(=O)C(CCCCCCCC)CCCCCCCC] |
| C | 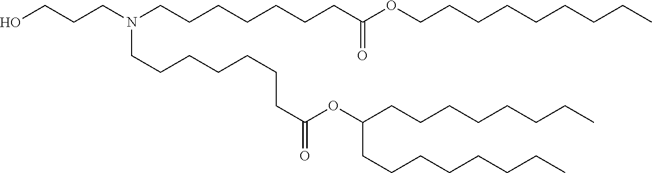 [SMILES: CCCCCCCCCOC(=O)CCCCCCCN(CCCO)CCCCCCCC(=O)OC(CCCCCCCC)CCCCCCCC] |
| | 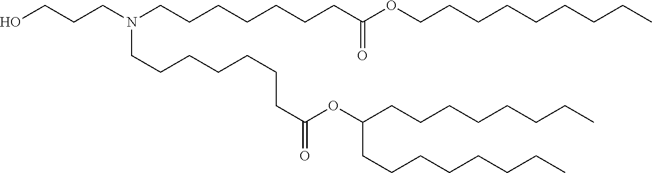 [SMILES: CCCCCCCCCOC(=O)CCCCCCCN(CCCO)CCCCCCCC(=O)OC(CCCCCCCC)CCCCCCCC] |
| D | 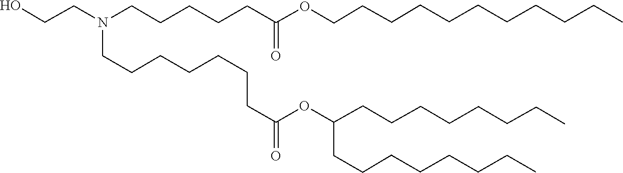 [SMILES: CCCCCCCCCCCOC(=O)CCCCCN(CCO)CCCCCCCC(=O)OC(CCCCCCCC)CCCCCCCC] |
| | 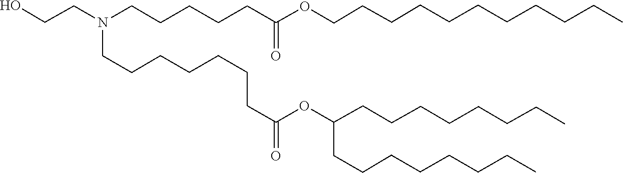 [SMILES: CCCCCCCCCCCOC(=O)CCCCCN(CCO)CCCCCCCC(=O)OC(CCCCCCCC)CCCCCCCC] |
| E | 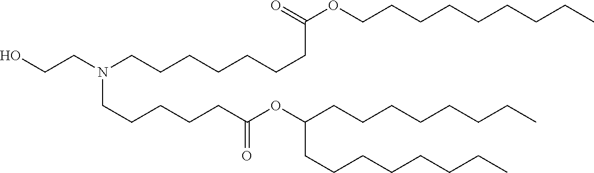 [SMILES: CCCCCCCCCOC(=O)CCCCCCCN(CCO)CCCCCC(=O)OC(CCCCCCCC)CCCCCCCC] |
| | 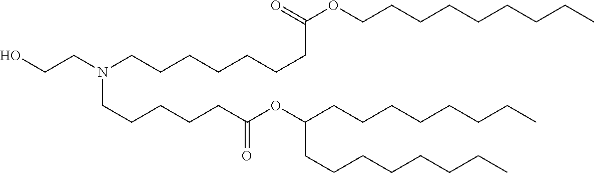 [SMILES: CCCCCCCCCOC(=O)CCCCCCCN(CCO)CCCCCC(=O)OC(CCCCCCCC)CCCCCCCC] |
| F | 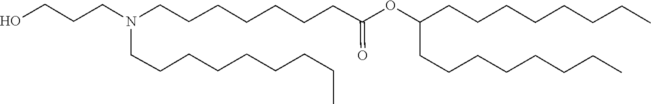 [SMILES: CCCCCCCCCN(CCCO)CCCCCCCC(=O)OC(CCCCCCCC)CCCCCCCC] |
| | 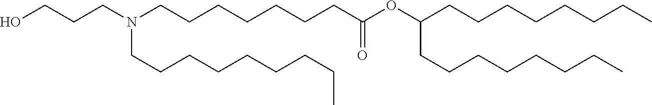 [SMILES: CCCCCCCCCN(CCCO)CCCCCCCC(=O)OC(CCCCCCCC)CCCCCCCC] |

In some embodiments, an LNP comprises a cationic lipid that is an ionizable lipid-like material (lipidoid). In some embodiments, a cationic lipid has the following structure:

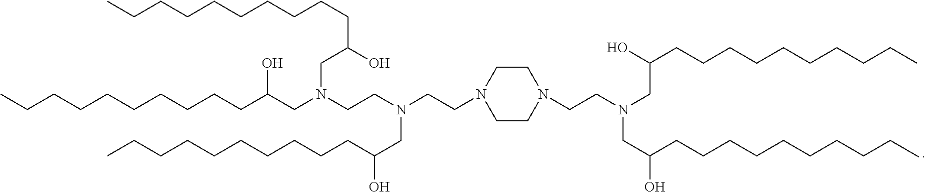 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC ; "."]

In some embodiments, lipid nanoparticles can have an average size (e.g., mean diameter) of about 30 nm to about 150 nm, about 40 nm to about 150 nm, about 50 nm to about 150 nm, about 60 nm to about 130 nm, about 70 nm to about 110 nm, about 70 nm to about 100 nm, about 70 to about 90 nm, or about 70 nm to about 80 nm. In some embodiments, lipid nanoparticles in accordance with the present disclosure can have an average size (e.g., mean diameter) of about 50 nm to about 100 nm. In some embodiments, lipid nanoparticles may have an average size (e.g., mean diameter) of about 50 nm to about 150 nm. In some embodiments, lipid nanoparticles may have an average size (e.g., mean diameter) of about 60 nm to about 120 nm. In some embodiments, lipid nanoparticles in accordance with the present disclosure can have an average size (e.g., mean diameter) of about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. The term “average diameter” or “mean diameter” refers to the mean hydrodynamic diameter of particles as measured by dynamic laser light scattering (DLS) with data analysis using the so-called cumulant algorithm, which provides as results the so-called Z-average with the dimension of a length, and the polydispersity index (PI), which is dimensionless (Koppel, D., J. Chem. Phys. 57, 1972, pp 4814-4820, ISO 13321, which is herein incorporated by reference). Here “average diameter,” “mean diameter,” “diameter,” or “size” for particles is used synonymously with this value of the Z-average.

In some embodiments, lipid nanoparticles described herein may exhibit a polydispersity index less than about 0.5, less than about 0.4, less than about 0.3, or about 0.2 or less. By way of example, lipid nanoparticles can exhibit a polydispersity index in a range of about 0.1 to about 0.3 or about 0.2 to about 0.3. The “polydispersity index” is preferably calculated based on dynamic light scattering measurements by the so-called cumulant analysis as mentioned in the definition of the “average diameter.” Under certain prerequisites, it can be taken as a measure of the size distribution of an ensemble of ribonucleic acid nanoparticles (e.g., ribonucleic acid nanoparticles).

Lipid nanoparticles described herein can be characterized by an “N/P ratio,” which is the molar ratio of cationic (nitrogen) groups (the “N” in N/P) in the cationic polymer to the anionic (phosphate) groups (the “P” in N/P) in RNA. It is understood that a cationic group is one that is either in cationic form (e.g., N+), or one that is ionizable to become cationic. Use of a single number in an N/P ratio (e.g., an N/P ratio of about 5) is intended to refer to that number over 1, e.g., an N/P ratio of about 5 is intended to mean 5:1. In some embodiments, a lipid nanoparticle described herein has an N/P ratio greater than or equal to 5. In some embodiments, a lipid nanoparticle described herein has an N/P ratio that is about 5, 6, 7, 8, 9, or 10. In some embodiments, an N/P ratio for a lipid nanoparticle described herein is from about 10 to about 50. In some embodiments, an N/P ratio for a lipid nanoparticle described herein is from about 10 to about 70. In some embodiments, an N/P ratio for a lipid nanoparticle described herein is from about 10 to about 120.

### B. Exemplary Methods of Making Lipid Nanoparticles

Lipids and lipid nanoparticles comprising nucleic acids and their method of preparation are known in the art, including, e.g., as described in U.S. Pat. Nos. 8,569,256, 5,965,542 and U.S. Patent Publication Nos. 2016/0199485, 2016/0009637, 2015/0273068, 2015/0265708, 2015/0203446, 2015/0005363, 2014/0308304, 2014/0200257, 2013/086373, 2013/0338210, 2013/0323269, 2013/0245107, 2013/0195920, 2013/0123338, 2013/0022649, 2013/0017223, 2012/0295832, 2012/0183581, 2012/0172411, 2012/0027803, 2012/0058188, 2011/0311583, 2011/0311582, 2011/0262527, 2011/0216622, 2011/0117125, 2011/0091525, 2011/0076335, 2011/0060032, 2010/0130588, 2007/0042031, 2006/0240093, 2006/0083780, 2006/0008910, 2005/0175682, 2005/017054, 2005/0118253, 2005/0064595, 2004/0142025, 2007/0042031, 1999/009076 and PCT Pub. Nos. WO 99/39741, WO 2018/081480, WO 2017/004143, WO 2017/075531, WO 2015/199952, WO 2014/008334, WO 2013/086373, WO 2013/086322, WO 2013/016058, WO 2013/086373, WO2011/141705, and WO 2001/07548, the full disclosures each of which are herein incorporated by reference in their entirety for the purposes described herein.

For example, in some embodiments, cationic lipids, neutral lipids (e.g., DSPC, and/or cholesterol) and polymer-conjugated lipids can be solubilized in ethanol at a pre-determined molar ratio (e.g., ones described herein). In some embodiments, lipid nanoparticles (lipid nanoparticle) are prepared at a total lipid to polyribonucleotides weight ratio of approximately 10:1 to 30:1. In some embodiments, such polyribonucleotides can be diluted to 0.2 mg/mL in acetate buffer.

In some embodiments, using an ethanol injection technique, a colloidal lipid dispersion comprising polyribonucleotides can be formed as follows: an ethanol solution comprising lipids, such as cationic lipids, neutral lipids, and polymer-conjugated lipids, is injected into an aqueous solution comprising polyribonucleotides (e.g., ones described herein).

In some embodiments, lipid and polyribonucleotide solutions can be mixed at room temperature by pumping each solution at controlled flow rates into a mixing unit, for example, using piston pumps. In some embodiments, the flow rates of a lipid solution and a RNA solution into a mixing unit are maintained at a ratio of 1:3. Upon mixing, nucleic acid-lipid particles are formed as the ethanolic lipid solution is diluted with aqueous polyribonucleotides. The lipid solubility is decreased, while cationic lipids bearing a positive charge interact with the negatively charged RNA.

In some embodiments, a solution comprising RNA-encapsulated lipid nanoparticles can be processed by one or more of concentration adjustment, buffer exchange, formulation, and/or filtration.

In some embodiments, RNA-encapsulated lipid nanoparticles can be processed through filtration.

In some embodiments, particle size and/or internal structure of lipid nanoparticles (with or without RNAs) may be monitored by appropriate techniques such as, e.g., small-angle X-ray scattering (SAXS) and/or transmission electron cryomicroscopy (CryoTEM).

### V. Pharmaceutical Compositions

The present disclosure provides compositions, e.g., pharmaceutical compositions comprising one or more polyribonucleotides described herein. Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, MD, 2006; incorporated herein by reference) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

In some embodiments, an excipient is approved for use in humans and for veterinary use. In some embodiments, an excipient is approved by the United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical formulations. Excipients such as cocoa butter and suppository waxes, coloring agents, coating agents, sweetening, flavoring, and/or perfuming agents can be present in the composition, according to the judgment of the formulator.

General considerations in the formulation and/or manufacture of pharmaceutical agents may be found, for example, in Remington: The Science and Practice of Pharmacy 21st ed., Lippincott Williams & Wilkins, 2005 (incorporated herein by reference).

In some embodiments, pharmaceutical compositions provided herein may be formulated with one or more pharmaceutically acceptable carriers or diluents as well as any other known adjuvants and excipients in accordance with conventional techniques such as those disclosed in Remington: The Science and Practice of Pharmacy 21st ed., Lippincott Williams & Wilkins, 2005 (incorporated herein by reference).

Pharmaceutical compositions described herein can be administered by appropriate methods known in the art. As will be appreciated by a skilled artisan, the route and/or mode of administration may depend on a number of factors, including, e.g., but not limited to stability and/or pharmacokinetics and/or pharmacodynamics of pharmaceutical compositions described herein.

In some embodiments, pharmaceutical compositions described herein are formulated for parenteral administration, which includes modes of administration other than enteral and topical administration, usually by injection, and includes, without limitation, intravenous, intramuscular, intraarterial, intradermal, subcutaneous, subcuticular, or intraarticular injection and infusion. In preferred embodiments, pharmaceutical compositions described herein are formulated for intravenous, intramuscular, or subcutaneous administration. In particularly preferred embodiments, pharmaceutical compositions described herein are formulated for intramuscular administration.

In some embodiments, pharmaceutical compositions described herein are formulated for intravenous administration. In some embodiments, pharmaceutically acceptable excipients that may be useful for intravenous administration include sterile aqueous solutions or dispersions and sterile powders for preparation of sterile injectable solutions or dispersions.

Therapeutic compositions typically must be sterile and stable under the conditions of manufacture and storage. The composition can be formulated as a solution, microemulsion, lipid nanoparticles, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. Proper fluidity can be maintained, for example, by the use of surfactants. In many cases, it will be preferable to include isotonic agents, for example, sugars, polyalcohols such as mannitol, sorbitol, or sodium chloride in the composition. In some embodiments, prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization and/or microfiltration. In some embodiments, pharmaceutical compositions can be prepared as described herein and/or methods known in the art. In some embodiments, a pharmaceutical composition includes ALC-0315; ALC-0159; DSPC; Cholesterol; Sucrose; NaCl; KCl; Na₂HPO₄; KH₂PO₄; Water for injection. In some embodiments, normal saline (isotonic 0.9% NaCl) is used as diluent.

These compositions may also contain adjuvants such as preservatives, wetting agents, emulsifying agents and dispersing agents. Prevention of the presence of microorganisms may be ensured both by sterilization procedures, and by the inclusion of various antibacterial and antifungal agents, for example, paraben, chlorobutanol, phenol sorbic acid, and the like. It may also be desirable to include isotonic agents, such as sugars, sodium chloride, and the like into pharmaceutical compositions described herein. In addition, prolonged absorption of the injectable pharmaceutical form may be brought about by the inclusion of agents which delay absorption such as aluminum monostearate and gelatin.

Formulations of pharmaceutical compositions described herein may be prepared by any method known or hereafter developed in the art of pharmacology. In general, such preparatory methods include the step of bringing active ingredient(s) into association with a diluent or another excipient and/or one or more other accessory ingredients, and then, if necessary and/or desirable, shaping and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” is discrete amount of the pharmaceutical composition comprising a predetermined amount of at least one RNA product produced using a system and/or method described herein.

Relative amounts of polyribonucleotides encapsulated in lipid nanoparticles, a pharmaceutically acceptable excipient, and/or any additional ingredients in a pharmaceutical composition can vary, depending upon the subject to be treated, target cells, diseases or disorders, and may also further depend upon the route by which the composition is to be administered.

In some embodiments, pharmaceutical compositions described herein are formulated into pharmaceutically acceptable dosage forms by conventional methods known to those of skill in the art. Actual dosage levels of the active ingredients (e.g., polyribonucleotides encapsulated in lipid nanoparticles) in the pharmaceutical compositions described herein may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present disclosure employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A physician having ordinary skill in the art can readily determine and prescribe the effective amount of the pharmaceutical composition required. For example, a physician could start doses of active ingredients (e.g., polyribonucleotides encapsulated in lipid nanoparticles) employed in the pharmaceutical composition at levels lower than that required in order to achieve the desired therapeutic effect and gradually increase the dosage until the desired effect is achieved.

In some embodiments, a pharmaceutical composition is formulated (e.g., but not limited to, for intravenous, intramuscular, or subcutaneous administration) to deliver a dose of about 5 mg RNA/kg.

In some embodiments, a pharmaceutical composition described herein may further comprise one or more additives, for example, in some embodiments that may enhance stability of such a composition under certain conditions. Examples of additives may include but are not limited to salts, buffer substances, preservatives, and carriers. For example, in some embodiments, a pharmaceutical composition may further comprise a cryoprotectant (e.g., sucrose) and/or an aqueous buffered solution, which may in some embodiments include one or more salts, including, e.g., alkali metal salts or alkaline earth metal salts such as, e.g., sodium salts, potassium salts, and/or calcium salts.

In some embodiments, a pharmaceutical composition provided herein is a preservative-free, sterile RNA-lipid nanoparticle dispersion in an aqueous buffer for intravenous or intramuscular administration.

Although the descriptions of pharmaceutical compositions provided herein are principally directed to pharmaceutical compositions that are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation.

### VI. Patient Populations

In some aspects, technologies of the present disclosure are used for therapeutic and/or prophylactic purposes. In some embodiments, technologies of the present disclosure are used in the treatment and/or prophylactic of an infection with a Plasmodium parasite. Prophylactic purposes of the present disclosure comprise pre-exposure prophylaxis and/or post-exposure prophylaxis. In some such embodiments, a Plasmodium parasite is, for example, Plasmodium falciparum, Plasmodium knowlesi, Plasmodium ovale, Plasmodium simiovale, Plasmodium vivax, Plasmodium malariae, Plasmodium ovale curtisi, Plasmodium ovale wallikeri, and/or Plasmodium berghei.

In some embodiments, technologies of the present disclosure are used in the treatment and/or prophylaxis of a disorder related to such an infection. A disordered related to such an infection comprises, for example, a typical symptom and/or a complication of a malaria infection.

In some embodiments, provided compositions (e.g., that are or comprise malarial antigens) may be useful to detect and/or characterize one or more features of an anti-malarial immune response (e.g., by detecting binding to a provided antigen by serum from an infected subject).

In some embodiments, provided compositions (e.g., that are or comprise malarial antigens) are useful to raise antibodies to one or more epitopes included therein; such antibodies may themselves be useful, for example for detection or treatment of malarial parasite(s) or infection thereby.

The present disclosure provides use of encoding nucleic acids (e.g., DNA or RNA) to produce encoded antigens and/or use of DNA constructs to produce RNA.

In some embodiments, technologies of the present disclosure are utilized in a non-limited subject population; in some embodiments, technologies of the present disclosure are utilized in particular subject populations.

In some embodiments, a subject population comprises an adult population. In some embodiments, an adult population comprises subjects between the ages of about 19 years and about 60 years of age (e.g., about 20, 25, 30, 35, 40, 45, 50, 55, or 60 years of age). In some embodiments, an adult population comprises subjects between the ages of about 19 years and about 60 years of age (e.g., about 20, 25, 30, 35, 40, 45, 50, 55, or 60 years of age). In some embodiments, an adult population comprises subjects between the ages of about 18 years and about 55 years of age (e.g., about 20, 25, 30, 35, 40, 45, 50, 55, or 60 years of age).

In some embodiments, a subject population comprises an elderly population. In some embodiments, an elderly population comprises subjects of about 60 years of age, about 70 years of age, or older (e.g., about 65, 70, 75, 80, 85, 90, 95, or 100 years of age).

In some embodiments, a subject population comprises a pediatric population. In some embodiments, a pediatric population comprises subjects approximately 18 years old or younger. In some such embodiments, a pediatric population comprises subjects between the ages of about 1 year and about 18 years (e.g., 1, 2, 3, 4, 5, 6, 7, 8, 9, 10, 11, 12, 13, 14, 15, 16, 17, or 18 years of age).

In some embodiments, a subject population comprises a newborn population. In some embodiments, a newborn population comprises subjects about 12 months or younger (e.g., 12, 11, 10, 9, 8, 7, 6, 5, 4, 3, 2, 1 months or younger). In some embodiments, subject populations to be treated with technologies described herein include infants (e.g., about 12 months or younger) whose mothers did not receive such technologies described herein during pregnancy. In some embodiments, subject populations to be treated with technologies described herein may include pregnant women; in some embodiments, infants whose mothers were treated with disclosed technologies during pregnancy (e.g., who received at least one dose, or alternatively only who received both doses), are not vaccinated during the first weeks, months, or even years (e.g., 1, 2, 3, 4, 5, 6, 7, 8 weeks or more, or 1, 2, 3, 4, 5, 6, 7, 8, 9, 10, 11, 12, 13, 14, 15, 16, 17, 18, 19, 20, 21, 22, 23, 24 months or more, or 1, 2, 3, 4, 5 years or more) post-birth. Alternatively or additionally, in some embodiments, infants whose mothers were treated with disclosed technologies during pregnancy (e.g., who received at least one dose, or alternatively only who received both doses), receive reduced treated with disclosed technologies (e.g., lower doses and/or smaller numbers of administrations—e.g., boosters- and/or lower total exposure over a given period of time) after birth, for example during the first weeks, months, or even years (e.g., 1, 2, 3, 4, 5, 6, 7, 8 weeks or more, or 1, 2, 3, 4, 5, 6, 7, 8, 9, 10, 11, 12, 13, 14, 15, 16, 17, 18, 19, 20, 21, 22, 23, 24 months or more, or 1, 2, 3, 4, 5 years or more) post-birth or may need reduced vaccination (e.g., lower doses and/or smaller numbers of administrations—e.g., boosters-over a given period of time), In some embodiments, compositions as provided herein are administered to subject populations that do not include pregnant women.

In some embodiments, a subject population is or comprises children aged 6 weeks to up to 17 months of age.

In some embodiments, a subject has a body mass index over 15 kg/m2 and under 40 kg/m2. In some embodiments, a subject has a body mass index over 18.5 kg/m2 and under 35 kg/m. In some embodiments, a subject's body mass index is determined at an initial visit with a health professional. In some embodiments, a subject's body mass index is determined when a first dose of a pharmaceutical composition (e.g., immunogenic composition, e.g., vaccine) as described herein is administered.

In some embodiments, a subject weighs at least 40 kg. In some embodiments, a subject weighs at least 45 kg. In some embodiments, a subject's weight is determined at an initial visit with a health professional. In some embodiments, a subject's weight is determined when a first dose of a pharmaceutical composition (e.g., immunogenic composition, e.g., vaccine) as described herein is administered.

In some embodiments, a subject has a body mass index over 15 kg/m2 and under 40 kg/m2 and weighs at least 40 kg. In some embodiments, a subject has a body mass index over 18.5 kg/m2 and under 35 kg/m2 and weighs at least 45 kg. In some embodiments, a subject's body mass index and weight are determined at an initial visit with a health professional. In some embodiments, a subject's body mass index and weight are determined when a first dose of a pharmaceutical composition (e.g., immunogenic composition, e.g., vaccine) as described herein is administered.

In some embodiments, a subject population comprises a population with a high risk of infection (e.g., Malaria). In some such embodiments, a population may be deemed to have a high risk of infection due to a local epidemic or a global pandemic. In some such embodiments, a population may be deemed to have a high risk of infection due to a subject population's geographic area. In some embodiments, a subject population comprises subjects that have been exposed to infection (e.g., Malaria).

In some embodiments, where a subject population is or includes pregnant women, provided technologies offer a particular advantage of interrupting malaria's transmission cycle, including, for example, in some embodiments, by reducing or eliminating transmission from pregnant mothers to their fetuses.

In some embodiments, a subject population is or comprises immunocompromised individuals. In some embodiments, a subject population does not include immunocompromised individuals.

In some embodiments, a provided pharmaceutical composition (e.g., immunogenic composition, e.g., vaccine) may be administered in combination with (i.e., so that subject(s) are simultaneously exposed to both) another pharmaceutical composition (e.g., immunogenic composition, e.g., vaccine) or therapeutic intervention, e.g., to treat or prevent malaria or another disease, disorder, or condition.

In some embodiments, a provided pharmaceutical composition (e.g., immunogenic composition, e.g., vaccine) may be administered with a protein vaccine, a DNA vaccine, an RNA vaccine, a cellular vaccine, a conjugate vaccine, etc. In some embodiments, one or more doses of a provided pharmaceutical composition (e.g., immunogenic composition, e.g., vaccine) may be administered together with (e.g., in a single visit) another vaccine or other therapy.

In some embodiments, a provided pharmaceutical composition (e.g., immunogenic composition, e.g., vaccine) may be administered to subjects who have been exposed, or expect they have been exposed, to malaria. In some embodiments, a provided pharmaceutical composition (e.g., immunogenic composition, e.g., vaccine) may be administered to subjects who do not have symptoms of malarial infection.

### VII. Treatment Methods

In some embodiments, technologies of the present disclosure may be administered to subjects according to a particular dosing regimen. In some embodiments, a dosing regimen may involve a single administration; in some embodiments, a dosing regimen may comprise one or more “booster” administrations after the initial administration. In some embodiments, initial and boost doses are the same amount; in some embodiments they differ. In some embodiments, two or more booster doses are administered. In some embodiments, a plurality of doses are administered at regular intervals. In some embodiments, periods of time between doses become longer. In some embodiments, one or more subsequent doses is administered if a particular clinical (e.g., reduction in neutralizing antibody levels) or situational (e.g., local development of a new strain) even arises or is detected.

In some embodiments, administered pharmaceutical compositions (e.g., immunogenic compositions, e.g., vaccines) comprising RNA constructs that encode Plasmodium polypeptide constructs are administered in RNA doses of from about 0.1 μg to about 300 μg, about 0.5 μg to about 200 μg, or about 1 μg to about 100 μg, such as about 1 μg, about 3 μg, about 10 μg, about 30 μg, about 50 μg, or about 100 μg. In some embodiments, an saRNA construct is administered at a lower dose (e.g., 2, 4, 5, 10 fold or more lower) than a modRNA or uRNA construct.

In some embodiments, a first booster dose is administered within a about six months of the initial dose, and preferably within about 5, 4, 3, 2, or 1 months. In some embodiments, a first booster dose is administered in a time period that begins about 1, 2, 3, or 4 weeks after the first dose, and ends about 2, 3, 4, 5, 6, 7, 8, 9, 10, 11, 12 weeks of the first dose (e.g., between about 1 and about 12 weeks after the first dose, or between about 2 or 3 weeks and about 5 and 6 weeks after the first dose, or about 3 weeks or about 4 weeks after the first dose).

In some embodiments, a plurality of booster doses (e.g., 2, 3, or 4) doses are administered within 6 months of the first dose, or within 12 months of the first dose.

In some embodiments, a first dose and a second dose are administered about 6, about 7, about 8, about 9, or about 10 weeks apart. In other words, in some embodiments, a second dose is administered about 6, about 7, about 8, about 9, or about 10 weeks after a first dose.

In some embodiments, a third dose is administered about 15, about 16, about 17, about 18, about 19, or about 20 weeks after a second dose.

In some embodiments, a second dose is administered about 8 weeks after a first dose, and a third dose is administered about 18 weeks after the second dose.

In some embodiments, 3 doses or fewer are required to achieve effective vaccination (e.g., greater than 60%, and in some embodiments greater than about 70%, about 75%, about 80%, about 85%, about 90% or more) reduction in risk of infection, or of serious disease. In some embodiments, not more than two doses are required. In some embodiments, a single dose is sufficient. In some embodiments, an RNA dose is about 60 μg or lower, 50 μg or lower, 40 μg or lower, 30 μg or lower, 20 μg or lower, 10 μg or lower, 5 μg or lower, 2.5 μg or lower, or 1 μg or lower. In some embodiments, an RNA dose is about 0.25 μg, at least 0.5 μg, at least 1 μg, at least 2 μg, at least 3 μg, at least 4 μg, at least 5 μg, at least 10 μg, at least 20 μg, at least 30 μg, or at least 40 μg. In some embodiments, an RNA dose is about 0.25 μg to 60 μg, 0.5 μg to 55 μg, 1 μg to 50 ug, 5 μg to 40 μg, or 10 μg to 30 μg may be administered per dose. In some embodiments, an RNA dose is about 30 μg. In some embodiments, at least two such doses are administered. For example, a second dose may be administered about 21 days following administration of the first dose. In some embodiments, a first booster dose is administered about one month after an initial dose. In some such embodiments, at least one further booster is administered at one-month interval(s). In some embodiments, after 2 or 3 boosters, a longer interval is introduced and no further booster is administered for at least 6, 9, 12, 18, 24, or more months. In some embodiments, a single further booster is administered after about 18 months. In some embodiments, no further booster is required unless, for example, a material change in clinical or environmental situation is observed.

In some embodiments, one or more outcomes may be assessed following administration of one or more doses of a pharmaceutical composition provided herein. Exemplary outcomes include, but are not limited to: local reaction at the injection site (e.g., pain, erythema/redness, induration/swelling, etc.), frequency of solicited local reactions at the injection site (e.g., pain, erythema/redness, induration/swelling, etc.), systemic reactions (e.g., vomiting, diarrhea, headache, fatigue, muscle/joint pain, fever, etc.), frequency of solicited systemic reactions (e.g., vomiting, diarrhea, headache, fatigue, muscle/joint pain, fever, etc.), adverse events, medically attended adverse events, severe adverse events, frequency of subjects with at least one adverse event, frequency of subjects with at least one medically attended adverse events, frequency of subjects with at least one severe adverse events, number of subjects protected from blood stage parasitemia, proportion of subjects protected from blood stage parasitemia, and combinations thereof.

In some embodiments, blood stage parasitemia can be assessed by PCR, e.g., qPCR.

In some embodiments, statistics on antibody levels at assessed time points can be obtained following administration of one or more doses of a pharmaceutical composition provided herein. Time points can include at the time of a first dose, the time of a second dose, the time of a third dose, following a known or suspected malaria exposure, or following a known or suspected malaria infection.

### VIII. Methods of Manufacture

Individual polyribonucleotides can be produced by methods known in the art. For example, in some embodiments, polyribonucleotides can be produced by in vitro transcription, for example, using a DNA template. A plasmid DNA used as a template for in vitro transcription to generate a polyribonucleotide described herein is also within the scope of the present disclosure.

A DNA template is used for in vitro RNA synthesis in the presence of an appropriate RNA polymerase (e.g., a recombinant RNA-polymerase such as a T7 RNA-polymerase) with ribonucleotide triphosphates (e.g., ATP, CTP, GTP, UTP). In some embodiments, polyribonucleotides (e.g., ones described herein) can be synthesized in the presence of modified ribonucleotide triphosphates. By way of example only, in some embodiments, pseudouridine (w), N₁-methyl-pseudouridine (m1ψ), or 5-methyl-uridine (m5U) can be used to replace uridine triphosphate (UTP). In some embodiments, pseudouridine (w) can be used to replace uridine triphosphate (UTP). In some embodiments, N₁-methyl-pseudouridine (m1ψ) can be used to replace uridine triphosphate (UTP). In some embodiments, 5-methyl-uridine (m5U) can be used to replace uridine triphosphate (UTP).

As will be clear to those skilled in the art, during in vitro transcription, an RNA polymerase (e.g., as described and/or utilized herein) typically traverses at least a portion of a single-stranded DNA template in the 3′ →5′ direction to produce a single-stranded complementary RNA in the 5′ →3′ direction.

In some embodiments where a polyribonucleotide comprises a polyA tail, one of those skill in the art will appreciate that such a polyA tail may be encoded in a DNA template, e.g., by using an appropriately tailed PCR primer, or it can be added to a polyribonucleotide after in vitro transcription, e.g., by enzymatic treatment (e.g., using a poly(A) polymerase such as an E. coli Poly(A) polymerase). Suitable poly(A) tails are described herein above. For example, in some embodiments, a poly(A) tail comprises a nucleotide sequence according to SEQ ID NO: 428. In some embodiments, a poly(A) tail comprises a plurality of A residues interrupted by a linker. In some embodiments, a linker comprises a nucleotide sequence according to SEQ ID NO: 430.

In some embodiments, those skilled in the art will appreciate that addition of a 5′ cap to an RNA (e.g., mRNA) can facilitate recognition and attachment of the RNA to a ribosome to initiate translation and enhances translation efficiency. Those skilled in the art will also appreciate that a 5′ cap can also protect an RNA product from 5′ exonuclease mediated degradation and thus increases half-life. Methods for capping are known in the art; one of ordinary skill in the art will appreciate that in some embodiments, capping may be performed after in vitro transcription in the presence of a capping system (e.g., an enzyme-based capping system such as, e.g., capping enzymes of vaccinia virus). In some embodiments, a cap may be introduced during in vitro transcription, along with a plurality of ribonucleotide triphosphates such that a cap is incorporated into a polyribonucleotide during transcription (also known as co-transcriptional capping). In some embodiments, a GTP fed-batch procedure with multiple additions in the course of the reaction may be used to maintain a low concentration of GTP in order to effectively cap the RNA. Suitable 5′ cap are described herein above. For example, in some embodiments, a 5′ cap comprises m7 (3′-OMeG) (5′) ppp (5′) (2′-OMeA) pG.

Following RNA transcription, a DNA template is digested. In some embodiments, digestion can be achieved with the use of DNase I under appropriate conditions.

In some embodiments, in-vitro transcribed polyribonucleotides may be provided in a buffered solution, for example, in a buffer such as HEPES, a phosphate buffer solution, a citrate buffer solution, an acetate buffer solution; in some embodiments, such solution may be buffered to a pH within a range of, for example, about 6.5 to about 7.5; in some embodiments approximately 7.0. In some embodiments, production of polyribonucleotides may further include one or more of the following steps: purification, mixing, filtration, and/or filling.

In some embodiments, polyribonucleotides can be purified (e.g., in some embodiments after in vitro transcription reaction), for example, to remove components utilized or formed in the course of the production, like, e.g., proteins, DNA fragments, and/or or nucleotides. Various nucleic acid purifications that are known in the art can be used in accordance with the present disclosure. Certain purification steps may be or include, for example, one or more of precipitation, column chromatography (including, e.g., but not limited to anionic, cationic, hydrophobic interaction chromatography (HIC)), solid substrate-based purification (e.g., magnetic bead-based purification). In some embodiments, polyribonucleotides may be purified using magnetic bead-based purification, which in some embodiments may be or comprise magnetic bead-based chromatography. In some embodiments, polyribonucleotides may be purified using hydrophobic interaction chromatography (HIC) and/or diafiltration. In some embodiments, polyribonucleotides may be purified using HIC followed by diafiltration.

In some embodiments, dsRNA may be obtained as side product during in vitro transcription. In some such embodiments, a second purification step may be performed to remove dsRNA contamination. For example, in some embodiments, cellulose materials (e.g., microcrystalline cellulose) may be used to remove dsRNA contamination, for examples in some embodiments in a chromatographic format. In some embodiments, cellulose materials (e.g., microcrystalline cellulose) can be pretreated to inactivate potential RNase contamination, for example in some embodiments by autoclaving followed by incubation with aqueous basic solution, e.g., NaOH. In some embodiments, cellulose materials may be used to purify polyribonucleotides according to methods described in WO 2017/182524, the entire content of which is incorporated herein by reference.

In some embodiments, a batch of polyribonucleotides may be further processed by one or more steps of filtration and/or concentration. For example, in some embodiments, polyribonucleotide(s), for example, after removal of dsRNA contamination, may be further subject to diafiltration (e.g., in some embodiments by tangential flow filtration), for example, to adjust the concentration of polyribonucleotides to a desirable RNA concentration and/or to exchange buffer to a drug substance buffer.

In some embodiments, polyribonucleotides may be processed through 0.2 μm filtration before they are filled into appropriate containers.

In some embodiments, polyribonucleotides and compositions thereof may be manufactured in accordance with a process as described herein, or as otherwise known in the art.

In some embodiments, polyribonucleotides and compositions thereof may be manufactured at a large scale. For example, in some embodiments, a batch of polyribonucleotides can be manufactured at a scale of greater than 1 g, greater than 2 g, greater than 3 g, greater than 4 g, greater than 5 g, greater than 6 g, greater than 7 g, greater than 8 g, greater than 9 g, greater than 10 g, greater than 15 g, greater than 20 g, or higher.

In some embodiments, RNA quality control may be performed and/or monitored at any time during production process of polyribonucleotides and/or compositions comprising the same. For example, in some embodiments, RNA quality control parameters, including one or more of RNA identity (e.g., sequence, length, and/or RNA natures), RNA integrity, RNA concentration, residual DNA template, and residual dsRNA, may be assessed and/or monitored after each or certain steps of a polyribonucleotide manufacturing process, e.g., after in vitro transcription, and/or each purification step.

In some embodiments, the stability of polyribonucleotides (e.g., produced by in vitro transcription) and/or compositions comprising polyribonucleotides can be assessed under various test storage conditions, for example, at room temperatures vs. fridge or sub-zero temperatures over a period of time (e.g., at least 3 months, at least 6 months, at least 9 months, at least 12 months, or longer). In some embodiments, polyribonucleotides (e.g., ones described herein) and/or compositions thereof may be stored stable at a fridge temperature (e.g., about 4° C. to about 10° C.) for at least 1 month or longer including, at least 2 months, at least 3 months, at least 4 months, at least 5 months, at least 6 months, at least 7 months, at least 8 months, at least 9 months, at least 10 months, at least 11 months, or at least 12 months or longer. In some embodiments, polyribonucleotides (e.g., ones described herein) and/or compositions thereof may be stored stable at a sub-zero temperature (e.g., −20° C. or below) for at least 1 month or longer including, at least 2 months, at least 3 months, at least 4 months, at least 5 months, at least 6 months, at least 7 months, at least 8 months, at least 9 months, at least 10 months, at least 11 months, or at least 12 months or longer. In some embodiments, polyribonucleotides (e.g., ones described herein) and/or compositions thereof may be stored stable at room temperature (e.g., at about 25° C.) for at least 1 month or longer.

In some embodiments, one or more assessments may be utilized during manufacture, or other preparation or use of polyribonucleotides (e.g., as a release test).

In some embodiments, one or more quality control parameters may be assessed to determine whether polyribonucleotides described herein meet or exceed acceptance criteria (e.g., for subsequent formulation and/or release for distribution). In some embodiments, such quality control parameters may include, but are not limited to RNA integrity, RNA concentration, residual DNA template and/or residual dsRNA. Certain methods for assessing RNA quality are known in the art; for example, one of skill in the art will recognize that in some embodiments, one or more analytical tests can be used for RNA quality assessment. Examples of such certain analytical tests may include but are not limited to gel electrophoresis, UV absorption, and/or PCR assay.

In some embodiments, a batch of polyribonucleotides may be assessed for one or more features as described herein to determine next action step(s). For example, a batch of polyribonucleotides can be designated for one or more further steps of manufacturing and/or formulation and/or distribution if RNA quality assessment indicates that such a batch of polyribonucleotides meet or exceed the relevant acceptance criteria. Otherwise, an alternative action can be taken (e.g., discarding the batch) if such a batch of polyribonucleotides does not meet or exceed the acceptance criteria.

In some embodiments, a batch of polyribonucleotides that satisfy assessment results can be utilized for one or more further steps of manufacturing and/or formulation and/or distribution.

### IX. DNA Constructs

Among other things, the present disclosure provides DNA constructs, for example that may encode one or more antibody agents as described herein, or components thereof. In some embodiments, DNA constructs provided by and/or utilized in accordance with the present disclosure are comprised in a vector.

Non-limiting examples of a vector include plasmid vectors, cosmid vectors, phage vectors such as lambda phage, viral vectors such as retroviral, adenoviral or baculoviral vectors, or artificial chromosome vectors such as bacterial artificial chromosomes (BAC), yeast artificial chromosomes (YAC), or P1 artificial chromosomes (PAC). In some embodiments, a vector is an expression vector. In some embodiments, a vector is a cloning vector. In general, a vector is a nucleic acid construct that can receive or otherwise become linked to a nucleic acid element of interest (e.g., a construct that is or encodes a payload, or that imparts a particular functionality, etc.).

Expression vectors, which may be plasmid or viral or other vectors, typically include an expressible sequence of interest (e.g., a coding sequence) that is functionally linked with one or more control elements (e.g., promoters, enhancers, transcription terminators, etc.). Typically, such control elements are selected for expression in a system of interest. In some embodiments, a system is ex vivo (e.g., an in vitro transcription system); in some embodiments, a system is in vivo (e.g., a bacterial, yeast, plant, insect, fish, vertebrate, mammalian cell or tissue, etc.).

Cloning vectors are generally used to modify, engineer, and/or duplicate (e.g., by replication in vivo, for example in a simple system such as bacteria or yeast, or in vitro, such as by amplification such as polymerase chain reaction or other amplification process). In some embodiments, a cloning vector may lack expression signals.

In many embodiments, a vector may include replication elements such as primer binding site(s) and/or origin(s) of replication. In many embodiments, a vector may include insertion or modification sites such as restriction endonuclease recognition sites and/or guide RNA binding sites, etc.

In some embodiments, a vector is a viral vector (e.g., an AAV vector). In some embodiments, a vector is a non-viral vector. In some embodiments, a vector is a plasmid.

Those skilled in the art are aware of a variety of technologies useful for the production of recombinant polynucleotides (e.g., DNA or RNA) as described herein. For example, restriction digestion, reverse transcription, amplification (e.g., by polymerase chain reaction), Gibson assembly, etc., are well established and useful tools and technologies. Alternatively or additionally, certain nucleic acids may be prepared or assembled by chemical and/or enzymatic synthesis. In some embodiments, a combination of known methods is utilized to prepare a recombinant polynucleotide.

In some embodiments, polynucleotide(s) of the present disclosure are included in a DNA construct (e.g., a vector) amenable to transcription and/or translation.

In some embodiments, an expression vector comprises a polynucleotide that encodes proteins and/or polypeptides of the present disclosure operatively linked to a sequence or sequences that control expression (e.g., promoters, start signals, stop signals, polyadenylation signals, activators, repressors, etc.). In some embodiments, a sequence or sequences that control expression are selected to achieve a desired level of expression. In some embodiments, more than one sequence that controls expression (e.g., promoters) are utilized. In some embodiments, more than one sequence that controls expression (e.g., promoters) are utilized to achieve a desired level of expression of a plurality of polynucleotides that encode a plurality proteins and/or polypeptides. In some embodiments, a plurality of recombinant proteins and/or polypeptides are expressed from the same vector (e.g., a bi-cistronic vector, a tri-cistronic vector, multi-cistronic). In some embodiments, a plurality of polypeptides are expressed, each of which is expressed from a separate vector.

In some embodiments, an expression vector comprising a polynucleotide of the present disclosure is used to produce a RNA and/or protein and/or polypeptide in a host cell. In some embodiments, a host cell may be in vitro (e.g., a cell line)—for example a cell or cell line (e.g., Human Embryonic Kidney (HEK cells), Chinese Hamster Ovary cells, etc.) suitable for producing polynucleotides of the present disclosure and proteins and/or polypeptides encoded by said polynucleotides.

In some embodiments, an expression vector is an RNA expression vector. In some embodiments, an RNA expression vector comprises a polynucleotide template used to produce a RNA in cell-free enzymatic mix. In some embodiments, an RNA expression vector comprising a polynucleotide template is enzymatically linearized prior to in vitro transcription. In some embodiments, a polynucleotide template is generated through PCR as a linear polynucleotide template. In some embodiments, a linearized polynucleotide is mixed with enzymes suitable for RNA synthesis, RNA capping and/or purification. In some embodiments, the resulting RNA is suitable for producing proteins encoded by the RNA.

A variety of methods are known in the art to introduce an expression vector into host cells. In some embodiments, a vector may be introduced into host cells using transfection. In some embodiments, transfection is completed, for example, using calcium phosphate transfection, lipofection, or polyethylenimine-mediated transfection. In some embodiments, a vector may be introduced into a host cell using transduction.

In some embodiments, transformed host cells are cultured following introduction of a vector into a host cell to allow for expression of said recombinant polynucleotides. In some embodiments, a transformed host cells are cultured for at least 12 hours, 16 hours, 20 hours, 24 hours, 28 hours, 32 hours, 36 hours 40 hours, 44 hours, 48 hours, 52 hours, 56 hours, 60 hours, 64 hours, 68 hours, 72 hours or longer. Transformed host cells are cultured in growth conditions (e.g., temperature, carbon-dioxide levels, growth medium) in accordance with the requirements of a host cell selected. A skilled artisan would recognize culture conditions for host cells selected are well known in the art.

### EXEMPLARY NUMBERED EMBODIMENTS

Embodiment 1. A polyribonucleotide encoding a polypeptide, wherein the polypeptide comprises one or more Plasmodium CSP polypeptide regions or portions thereof.

Embodiment 2. The polyribonucleotide of embodiment 1, wherein each of the one or more Plasmodium CSP polypeptide regions or portions thereof comprise 25 or more contiguous amino acids of the amino acid sequence according to SEQ ID NO: 1.

Embodiment 3. The polyribonucleotide of embodiment 1 or 2, wherein the polypeptide comprises one or more repeats of the amino acid sequence of NANPNVDP, and wherein the polypeptide does not comprise the amino acid sequence of NPNA.

Embodiment 4. The polyribonucleotide of embodiment 1 or 2, wherein the polypeptide comprises five or more repeats of the amino acid sequence of NANPNVDP.

Embodiment 5. A polyribonucleotide encoding a polypeptide, wherein the polypeptide comprises:
(i) a heterologous secretory signal, and
(ii) one or more Plasmodium CSP polypeptide regions or portions thereof.

Embodiment 6. A polyribonucleotide encoding a polypeptide, wherein the polypeptide comprises:
(i) one or more Plasmodium CSP polypeptide regions or portions thereof, and
(ii) a heterologous transmembrane region.

Embodiment 7. The polyribonucleotide of any one of embodiments 1, 2, 5, and 6, wherein the polypeptide comprises one or more repeats of the amino acid sequence of NANPNVDP.

Embodiment 8. The polyribonucleotide of any one of embodiments 1-3 and 5-7, wherein the polypeptide comprises two or more repeats of the amino acid sequence of NANPNVDP.

Embodiment 9. The polyribonucleotide of any one of embodiments 1-3 and 5-8, wherein the polypeptide comprises between two and twelve repeats of the amino acid sequence of NANPNVDP.

Embodiment 10. The polyribonucleotide of any one of embodiments 1-3 and 5-9, wherein the polypeptide comprises exactly three repeats of the amino acid sequence of NANPNVDP.

Embodiment 11. The polyribonucleotide of any one of embodiments 1-3 and 5-9, wherein the polypeptide comprises between four and twelve repeats of the amino acid sequence of NANPNVDP.

Embodiment 12. The polyribonucleotide of any one of embodiments 1-9 and 11, wherein the polypeptide comprises:
(i) exactly eight repeats of the amino acid sequence of NANPNVDP; or
(ii) exactly nine repeats of the amino acid sequence of NANPNVDP.

Embodiment 13. The polyribonucleotide of any one of embodiments 3, 4, and 7-12, wherein the repeats of the amino acid sequence of NANPNVDP are all contiguous with each other.

Embodiment 14. The polyribonucleotide of any one of embodiments 3, 4, and 7-12, wherein the repeats of the amino acid sequence of NANPNVDP are not all contiguous with each other.

Embodiment 15. The polyribonucleotide of embodiment 14, wherein the polypeptide comprises three portions of a Plasmodium CSP polypeptide, and wherein each portion comprises three contiguous repeats of the amino acid sequence of NANPNVDP, wherein each of the portions are not contiguous with each other.

Embodiment 16. The polyribonucleotide of any one of embodiments 1-9, 11, 12, and 14, wherein the polypeptide comprises four portions of a Plasmodium CSP polypeptide, and wherein each portion comprises two contiguous repeats of the amino acid sequence of NANPNVDP.

Embodiment 17. The polyribonucleotide of any one of embodiments 1-15, wherein the polypeptide comprises one or more Plasmodium CSP C-terminal regions or portions thereof.

Embodiment 18. The polyribonucleotide of embodiment 17, wherein the polypeptide comprises exactly one Plasmodium CSP C-terminal region, and wherein the Plasmodium CSP C-terminal region comprises or consists of an amino acid sequence with at least 85% sequence identity to an amino acid sequence according to SEQ ID NO: 1.

Embodiment 19. The polyribonucleotide of embodiment 17, wherein the polypeptide comprises two or more portions of a Plasmodium CSP C-terminal region.

Embodiment 20. The polyribonucleotide of embodiment 17 or 19, wherein the polypeptide comprises one or more portions of the Plasmodium CSP C-terminal region, wherein each of the one or more portions comprise or consist of:
(i) an amino acid sequence according to SEQ ID NO: 111,
(ii) an amino acid sequence according to SEQ ID NO: 114,
(iii) an amino acid sequence according to SEQ ID NO: 117,
(iv) an amino acid sequence according to SEQ ID NO: 120, or
(v) a combination thereof.

Embodiment 21. The polyribonucleotide of embodiment 17 or 19, wherein the polypeptide comprises one portion of the Plasmodium CSP C-terminal region, wherein the portion comprises or consists of:
(i) an amino acid sequence according to SEQ ID NO: 111,
(ii) an amino acid sequence according to SEQ ID NO: 114,
(iii) an amino acid sequence according to SEQ ID NO: 117,
(iv) an amino acid sequence according to SEQ ID NO: 120, or
(v) a combination thereof.

Embodiment 22. The polyribonucleotide of embodiment 17 or 19, wherein the polypeptide comprises one or more portions of the Plasmodium CSP C-terminal region, wherein the one or more portions collectively comprise or consist of:
(i) an amino acid sequence according to SEQ ID NO: 111,
(ii) an amino acid sequence according to SEQ ID NO: 114,
(iii) an amino acid sequence according to SEQ ID NO: 117, and
(iv) an amino acid sequence according to SEQ ID NO: 120.

Embodiment 23. The polyribonucleotide of any one of embodiments 17-22, wherein the polypeptide comprises a serine immediately following the Plasmodium CSP C-terminal region.

Embodiment 24. The polyribonucleotide of any one of embodiments 17-22, wherein the polypeptide comprises a serine-valine sequence immediately following the Plasmodium CSP C-terminal region.

Embodiment 25. The polyribonucleotide of any one of embodiments 1-24, wherein the polypeptide comprises one or more Plasmodium CSP junction regions or portions thereof.

Embodiment 26. The polyribonucleotide of embodiment 25, wherein the polypeptide comprises two or more Plasmodium CSP junction regions or portions thereof.

Embodiment 27. The polyribonucleotide of embodiment 25, wherein the polypeptide comprises exactly one Plasmodium CSP junction region.

Embodiment 28. The polyribonucleotide of embodiment 25 or 27, wherein the Plasmodium CSP junction region consists of an amino acid sequence according to SEQ ID NO: 126.

Embodiment 29. The polyribonucleotide of embodiment 25 or 27, wherein the polypeptide comprises one or more portions of a Plasmodium CSP junction region.

Embodiment 30. The polyribonucleotide of embodiment 25 or 29, wherein the one or more portions of a Plasmodium CSP junction region comprise a deletion of one or more of K93, L⁹⁴, K95, Q96 and P97, and wherein the amino acid numbering is relative to SEQ ID NO: 1.

Embodiment 31. The polyribonucleotide of any one of embodiments 25, 29, and 30, wherein the one or more portions of a Plasmodium CSP junction region comprise a deletion of K93, L⁹⁴, K95, and Q96, and wherein the amino acid numbering is relative to SEQ ID NO: 1.

Embodiment 32. The polyribonucleotide of any one of embodiments 25 and 29-31, wherein the one or more portions of a Plasmodium CSP junction region comprise a deletion of K93, L⁹⁴, K95, Q96 and P97, and wherein the amino acid numbering is relative to SEQ ID NO: 1.

Embodiment 33. The polyribonucleotide of any one of embodiments 25, and 29-31, wherein each portion of a Plasmodium CSP junction region comprises or consists of an amino acid sequence according to SEQ ID NO: 129.

Embodiment 34. The polyribonucleotide of any one of embodiments 25, 29, 30, and 32, wherein each portion of a Plasmodium CSP junction region comprises or consists of an amino acid sequence according to SEQ ID NO: 132.

Embodiment 35. The polyribonucleotide of embodiment 26, wherein the two or more Plasmodium CSP junction regions consist of an amino acid sequence according to SEQ ID NO: 126.

Embodiment 36. The polyribonucleotide of embodiment 35, wherein the polypeptide comprises two or more portions of a Plasmodium CSP junction region.

Embodiment 37. The polyribonucleotide of embodiment 36, wherein the two or more portions of a Plasmodium CSP junction region comprise a deletion of one or more of K93, L⁹⁴, K95, Q96 and P97, and wherein the amino acid numbering is relative to SEQ ID NO: 1.

Embodiment 38. The polyribonucleotide of embodiment 36 or 37, wherein the two or more portions of a Plasmodium CSP junction region comprise a deletion of K93, L⁹⁴, K95, and Q96, and wherein the amino acid numbering is relative to SEQ ID NO: 1.

Embodiment 39. The polyribonucleotide of any one of embodiments 36-38, wherein the two or more portions of a Plasmodium CSP junction region comprise a deletion of K93, L⁹⁴, K95, Q96 and P97, and wherein the amino acid numbering is relative to SEQ ID NO: 1.

Embodiment 40. The polyribonucleotide of any one of embodiments 36-38, wherein each portion of a Plasmodium CSP junction region comprises or consists of an amino acid sequence according to SEQ ID NO: 129.

Embodiment 41. The polyribonucleotide of any one of embodiments 36, 37, and 39, wherein each portion of a Plasmodium CSP junction region comprises or consists of an amino acid sequence according to SEQ ID NO: 132.

Embodiment 42. The polyribonucleotide of any one of embodiments 1-24, wherein the polypeptide comprises one or more Plasmodium CSP junction region variants.

Embodiment 43. The polyribonucleotide of embodiment 42, wherein the Plasmodium CSP junction region variant comprises one or more substitution mutations.

Embodiment 44. The polyribonucleotide of embodiment 43, wherein the one or more substitution mutations comprise a K93A mutation, an L⁹⁴A mutation, or both, wherein the amino acid numbering is relative to SEQ ID NO: 1.

Embodiment 45. The polyribonucleotide of embodiment 43 or 44, wherein each Plasmodium CSP junction region variant comprises the amino acid sequence of SEQ ID NO: 426 (AAKQ).

Embodiment 46. The polyribonucleotide of any one of embodiments 1-45, wherein the polypeptide comprises one or more Plasmodium CSP N-terminal end regions or portions thereof.

Embodiment 47. The polyribonucleotide of embodiment 46, wherein the polypeptide comprises two or more Plasmodium CSP N-terminal end regions or portions thereof.

Embodiment 48. The polyribonucleotide of embodiment 46 or 47, wherein each Plasmodium CSP N-terminal end region consists of an amino acid sequence according to SEQ ID NO: 135.

Embodiment 49. The polyribonucleotide of any one of embodiments 1-48, wherein the polypeptide does not comprise a Plasmodium CSP N-terminal end region or any portion thereof.

Embodiment 50. The polyribonucleotide of any one of embodiments 1-49, wherein the polypeptide comprises one or more Plasmodium CSP N-terminal regions or portions thereof.

Embodiment 51. The polyribonucleotide of embodiment 50, wherein the polypeptide comprises two or more Plasmodium CSP N-terminal regions or portions thereof.

Embodiment 52. The polyribonucleotide of embodiment 50 or 51, wherein each Plasmodium CSP N-terminal region comprises or consists of an amino acid sequence with at least 85% sequence identity to an amino acid sequence according to SEQ ID NO: 138.

Embodiment 53. The polyribonucleotide of any one of embodiments 1-49, wherein the polypeptide does not comprise a Plasmodium CSP N-terminal region or any portion thereof.

Embodiment 54. The polyribonucleotide of any one of embodiments 1, 2, and 4-53, wherein the polypeptide comprises one or more Plasmodium CSP major repeat regions or portions thereof.

Embodiment 55. The polyribonucleotide of embodiment 54, wherein the one or more Plasmodium CSP major repeat regions or portions thereof comprise the amino acid sequence NANPNA or NPNANP.

Embodiment 56. The polyribonucleotide of embodiment 54 or 55, wherein the polypeptide comprises at least two Plasmodium CSP major repeat region portions, wherein each CSP major repeat region portion comprises at least 4 and at most 7 repeats of the sequence NANP.

Embodiment 57. The polyribonucleotide of any one of embodiments 54-56, wherein the polypeptide comprises two or three Plasmodium CSP major repeat region portions, wherein each CSP major repeat region portion comprises 6 repeats of the sequence NANP.

Embodiment 58. The polyribonucleotide of embodiment 54, wherein the polypeptide comprises exactly one Plasmodium CSP major repeat region or portion thereof, and the Plasmodium CSP major repeat region or portion thereof comprises a total of at least 2 and at most 35 repeats of the amino acid sequence NANP.

Embodiment 59. The polyribonucleotide of embodiment 58, wherein the Plasmodium CSP major repeat region or portion thereof comprises two contiguous stretches of repeats of the amino acid sequence NANP, and wherein the two contiguous stretches of repeats of the amino acid sequence NANP flank an amino acid sequence of NVDP.

Embodiment 60. The polyribonucleotide of embodiment 59, wherein the Plasmodium CSP major repeat region comprises, in N-terminus to C-terminus order, 17 repeats of the amino acid sequence NANP, an amino acid sequence of NVDP, and 18 repeats of the amino acid sequence NANP.

Embodiment 61. The polyribonucleotide of embodiment 58, wherein a portion of the Plasmodium CSP major repeat region consists of at most 18 contiguous repeats of the amino acid sequence NANP.

Embodiment 62. The polyribonucleotide of embodiment 58, wherein a portion of the Plasmodium CSP major repeat region consists of 2 contiguous repeats of the amino acid sequence NANP.

Embodiment 63. The polyribonucleotide of embodiment 58, wherein the Plasmodium CSP major repeat region comprises or consists of an amino acid sequence with at least 85% sequence identity to an amino acid sequence according to SEQ ID NO: 156.

Embodiment 64. The polyribonucleotide of any one of embodiments 1, 2, and 4-57, wherein the polypeptide regions comprise in N-terminus to C-terminus order:
(i) three contiguous repeats of the amino acid sequence NANPNVDP;
(ii) six contiguous repeats of the amino acid sequence NANP;
(iii) three contiguous repeats of the amino acid sequence NANPNVDP;
(iv) six contiguous repeats of the amino acid sequence NANP; and
(v) three contiguous repeats of the amino acid sequence NANPNVDP.

Embodiment 65. The polyribonucleotide of embodiment 64, further comprising:
(i) one or more Plasmodium CSP N-terminal regions or portions thereof,
(ii) one or more Plasmodium CSP N-terminal end regions or portions thereof,
(iii) one or more Plasmodium CSP junction regions, portions thereof, or variants thereof,
(iv) one or more Plasmodium CSP C-terminal regions or portions thereof, or
(v) a combination thereof.

Embodiment 66. The polyribonucleotide of embodiment 65, wherein the polypeptide regions comprise in N-terminus to C-terminus order:
(i) a Plasmodium CSP N-terminal region or portion thereof,
(ii) a Plasmodium CSP R¹ region or portion thereof,
(iii) a Plasmodium CSP junction region or portion thereof,
(iv) three contiguous repeats of the amino acid sequence NANPNVDP;
(v) six contiguous repeats of the amino acid sequence NANP;
(vi) three contiguous repeats of the amino acid sequence NANPNVDP;
(vii) six contiguous repeats of the amino acid sequence NANP;
(viii) three contiguous repeats of the amino acid sequence NANPNVDP; and
(ix) a Plasmodium CSP C-terminal region or portion thereof.

Embodiment 67. The polyribonucleotide of embodiment 66, further comprising a Plasmodium CSP

GPI domain.

Embodiment 68. The polyribonucleotide of embodiment 66 or 67, wherein the Plasmodium CSP C-terminal region or portion thereof comprises a substitution at a fucosylation site.

Embodiment 69. The polyribonucleotide of any one of embodiments 1, 2, 4, 7-9, 11-12, 14-15, and 25-57, wherein the polypeptide regions comprise in N-terminus to C-terminus order three repeating domains, wherein each repeating domain comprises:
(i) one or more Plasmodium CSP junction regions, portions thereof, or variants thereof;
(ii) three contiguous repeats of the amino acid sequence NANPNVDP; and
(iii) six contiguous repeats of the amino acid sequence NANP.

Embodiment 70. The polyribonucleotide of any one of embodiments 1, 2, 4, 7-9, 11-12, 14-15, and 25-57, wherein the polypeptide regions comprise in N-terminus to C-terminus order three repeating domains, wherein each repeating domain comprises:
(i) one or more Plasmodium R¹ regions or portions thereof,
(ii) one or more Plasmodium CSP junction regions, portions thereof, or variants thereof;
(iii) three contiguous repeats of the amino acid sequence NANPNVDP; and
(iv) six contiguous repeats of the amino acid sequence NANP.

Embodiment 71. The polyribonucleotide of embodiment 69 or 70, further comprising one or more linker sequences.

Embodiment 72. The polyribonucleotide of embodiment 71, wherein the linker is a gly-ser linker.

Embodiment 73. The polyribonucleotide of embodiment 71 or 72, wherein the polypeptide comprises a linker sequence following each six contiguous repeats of the amino acid sequence NANP.

Embodiment 74. The polyribonucleotide of any one of embodiments 69-73, wherein the polypeptide does not comprise (i) a Plasmodium CSP N-terminal region or any portion thereof and/or (ii) a Plasmodium CSP C-terminal region or any portion thereof.

Embodiment 75. The polyribonucleotide of any one of embodiments 1, 2, and 4-53, wherein the polypeptide does not comprise a Plasmodium CSP major repeat region or a portion of a Plasmodium CSP major repeat region comprising the amino acid sequence NPNA.

Embodiment 76. The polyribonucleotide of any one of embodiments 1-75, wherein the one or more Plasmodium CSP polypeptide regions or portions thereof, if present, are in the following N-terminus to C-terminus order:
(i) one or more Plasmodium CSP N-terminal regions or portions thereof,
(ii) one or more Plasmodium CSP N-terminal end regions or portions thereof,
(iii) one or more Plasmodium CSP junction regions, portions thereof, or variants thereof,
(iv) one or more repeats of the amino acid sequence of NANPNVDP,
(v) one or more Plasmodium CSP major repeat regions or portions thereof, and
(vi) one or more Plasmodium CSP C-terminal regions or portions thereof.

Embodiment 77. The polyribonucleotide of any one of embodiments 1-76, wherein the one or more Plasmodium CSP polypeptide regions or portions thereof, if present, are in the following N-terminus to C-terminus order:
(i) one Plasmodium CSP N-terminal region or portion thereof,
(ii) one Plasmodium CSP N-terminal end region or portion thereof,
(iii) one Plasmodium CSP junction region, portion thereof, or variant thereof,
(iv) one or more repeats of the amino acid sequence of NANPNVDP,
(v) one Plasmodium CSP major repeat region or portion thereof, and
(vi) one Plasmodium CSP C-terminal region or portion thereof.

Embodiment 78. The polyribonucleotide of any one of embodiments 1-77, wherein the polypeptide comprises one or more helper antigens.

Embodiment 79. The polyribonucleotide of embodiment 78, wherein the one or more helper antigens comprise a Plasmodium antigen.

Embodiment 80. The polyribonucleotide of embodiment 78 or 79, wherein the one or more helper antigens are Plasmodium 2-phospho-D-glycerate hydro-lyase antigen, Plasmodium liver stage antigen 1 (a), (LSA-1 (a)), Plasmodium liver stage antigen 1 (b) (LSA-1 (b)), Plasmodium thrombospondin-related anonymous protein (TRAP), Plasmodium liver stage associated protein 1 (LSAP1), Plasmodium liver stage associated protein 2 (LSAP2), Plasmodium UIS3, Plasmodium UIS4, Plasmodium ETRAMP10.3, Plasmodium liver specific protein 1 (LISP-1), Plasmodium liver specific protein 2 (LISP-2), Plasmodium liver stage antigen 3 (LSA-3), Plasmodium EXP1, Plasmodium E140, Plasmodium reticulocyte-binding protein homolog 5 (Rh5), Plasmodium glutamic acid-rich protein (GARP), Plasmodium parasite-infected erythrocyte surface protein 2 (PIESP2), Plasmodium Cysteine-Rich Protective Antigen (CyRPA), Plasmodium Ripr, Plasmodium P113, or a combination thereof.

Embodiment 81. The polyribonucleotide of any one of embodiments 78-80, wherein the one or more helper antigens comprise or consist of a P. falciparum 2-phospho-D-glycerate hydro-lyase antigen.

Embodiment 82. The polyribonucleotide of 81, wherein the P. falciparum 2-phospho-D-glycerate hydro-lyase antigen comprises or consists of an amino acid sequence according to SEQ ID NO: 240.

Embodiment 83. The polyribonucleotide of any one of embodiments 78-82, wherein the one or more helper antigens comprise or consist of a P. falciparum liver-stage antigen 3.

Embodiment 84. The polyribonucleotide of 83, wherein the P. falciparum liver-stage antigen 3 comprises or consists of an amino acid sequence according to SEQ ID NO: 243.

Embodiment 85. The polyribonucleotide of embodiment 78, wherein the one or more helper antigens comprise an Anopheles antigen.

Embodiment 86. The polyribonucleotide of embodiment 78 or 85, wherein the helper antigen comprises or consists of an Anopheles gambiae TRIO.

Embodiment 87. The polyribonucleotide of 86, wherein the Anopheles gambiae TRIO comprises or consists of an amino acid sequence according to SEQ ID NO: 246.

Embodiment 88. The polyribonucleotide of any one of embodiments 78-87, wherein the polypeptide comprises a secretory signal and the helper antigen immediately follows the secretory signal.

Embodiment 89. The polyribonucleotide of any one of embodiments 78-88, wherein the polypeptide comprises a helper antigen at the C-terminus of the polypeptide.

Embodiment 90. The polyribonucleotide of any one of embodiments 1-89, wherein the polypeptide comprises a multimerization region.

Embodiment 91. The polyribonucleotide of embodiment 90, wherein the multimerization region comprises or consists of a trimerization region.

Embodiment 92. The polyribonucleotide of embodiment 91, wherein the trimerization region comprises or consists of a fibritin region.

Embodiment 93. The polyribonucleotide of embodiment 92, wherein the fibritin region comprises or consists of an amino acid sequence according to SEQ ID NO: 255.

Embodiment 94. The polyribonucleotide of any one of embodiments 90-93, wherein the polypeptide comprises a multimerization region at the N-terminus of the polypeptide.

Embodiment 95. The polyribonucleotide of any one of embodiments 1˜4 and 6-87 and 89-94, wherein the polypeptide comprises a secretory signal.

Embodiment 96. The polyribonucleotide of embodiment 95, wherein the secretory signal comprises or consists a Plasmodium secretory signal.

Embodiment 97. The polyribonucleotide of embodiment 96, wherein the Plasmodium secretory signal comprises or consists of a Plasmodium CSP secretory signal.

Embodiment 98. The polyribonucleotide of embodiment 97, wherein the Plasmodium CSP secretory signal comprises or consists of an amino acid sequence according to SEQ ID NO: 174.

Embodiment 99. The polyribonucleotide of embodiment 95, wherein the secretory signal comprises or consists of a heterologous secretory signal.

Embodiment 100. The polyribonucleotide of 99, wherein the heterologous secretory signal comprises or consists of a non-human secretory signal.

Embodiment 101. The polyribonucleotide of embodiment 99 or 100, wherein the heterologous secretory signal comprises or consists of a viral secretory signal.

Embodiment 102. The polyribonucleotide of embodiment 101, wherein the viral secretory signal comprises or consists of an HSV secretory signal.

Embodiment 103. The polyribonucleotide of embodiment 102, wherein the HSV secretory signal comprises or consists of an HSV-1 or HSV-2 secretory signal.

Embodiment 104. The polyribonucleotide of embodiment 102 or 103, wherein the HSV secretory signal comprises or consists of an HSV glycoprotein D (gD) secretory signal.

Embodiment 105. The polyribonucleotide of embodiment 105, wherein the HSV gD secretory signal comprises or consists of an amino acid sequence according to SEQ ID NO: 159.

Embodiment 106. The polyribonucleotide of embodiment 105, wherein the HSV gD secretory signal comprises or consists of an amino acid sequence according to SEQ ID NO: 165.

Embodiment 107. The polyribonucleotide of embodiment 101, wherein the secretory signal comprises or consists of an Ebola virus secretory signal.

Embodiment 108. The polyribonucleotide of embodiment 107, wherein the Ebola virus secretory signal comprises or consists of an Ebola virus spike glycoprotein (SGP) secretory signal.

Embodiment 109. The polyribonucleotide of embodiment 108, wherein the Ebola virus SGP secretory signal comprises or consists of an amino acid sequence according to SEQ ID NO: 177.

Embodiment 110. The polyribonucleotide of any one of embodiments 95-109, wherein the secretory signal is located at the N-terminus of the polypeptide.

Embodiment 111. The polyribonucleotide of any one of embodiments 1-5 and 7-110, wherein the polypeptide comprises a transmembrane region.

Embodiment 112. The polyribonucleotide of embodiment 111, wherein the transmembrane region comprises or consists of a Plasmodium transmembrane region.

Embodiment 113. The polyribonucleotide of embodiment 112, wherein the Plasmodium transmembrane region comprises or consists of a Plasmodium CSP glycosylphosphatidylinositol (GPI) anchor region.

Embodiment 114. The polyribonucleotide of embodiment 113, wherein the Plasmodium CSP GPI anchor region comprises or consists of an amino acid sequence according to SEQ ID NO: 231.

Embodiment 115. The polyribonucleotide of embodiment 111, wherein the transmembrane region comprises or consists of a heterologous transmembrane region.

Embodiment 116. The polyribonucleotide of embodiment 115, wherein the heterologous transmembrane region does not comprise a hemagglutinin transmembrane region.

Embodiment 117. The polyribonucleotide of embodiment 115 or 116, wherein the heterologous transmembrane region comprises or consists of a non-human transmembrane region.

Embodiment 118. The polyribonucleotide of any one of embodiments 115-117, wherein the heterologous transmembrane region comprises or consists of a viral transmembrane region.

Embodiment 119. The polyribonucleotide of any one of embodiments 115-118, wherein the heterologous transmembrane region comprises or consists of an HSV transmembrane region.

Embodiment 120. The polyribonucleotide of embodiment 119, wherein the HSV transmembrane region comprises or consists of an HSV-1 or HSV-2 transmembrane region.

Embodiment 121. The polyribonucleotide of embodiment 119 or 120, wherein the HSV transmembrane region comprises or consists of an HSV gD transmembrane region.

Embodiment 122. The polyribonucleotide of embodiment 121, wherein the HSV gD transmembrane region comprises or consists of an amino acid sequence according to SEQ ID NO: 234.

Embodiment 123. The polyribonucleotide of embodiment 115 or 116, wherein the heterologous transmembrane region comprises or consists of a human transmembrane region.

Embodiment 124. The polyribonucleotide of embodiment 123, wherein the human transmembrane region comprises or consists of a human decay accelerating factor glycosylphosphatidylinositol (hDAF-GPI) anchor region.

Embodiment 125. The polyribonucleotide of embodiment 124, wherein the hDAF-GPI anchor region comprises or consists of an amino acid sequence according to SEQ ID NO: 237.

Embodiment 126. The polyribonucleotide of any one of embodiments 1-4, 6-87, 89-94, and 111-125, wherein the polypeptide does not comprise a secretory signal.

Embodiment 127. The polyribonucleotide of any one of embodiments 1-5 and 7-110, wherein the polypeptide does not comprise a transmembrane region.

Embodiment 128. The polyribonucleotide of any one of embodiments 1-127, wherein the polypeptide comprises one or more linkers.

Embodiment 129. The polyribonucleotide of embodiment 128, wherein the one or more linkers comprise or consist of an amino acid sequence according to SEQ ID NO: 258.

Embodiment 130. The polyribonucleotide of embodiment 128, wherein the one or more linkers comprise or consist of an amino acid sequence according to SEQ ID NO: 279.

Embodiment 131. The polyribonucleotide of embodiment 128, wherein the one or more linkers comprise or consist of an amino acid sequence according to SEQ ID NO: 270.

Embodiment 132. The polyribonucleotide of embodiment 128, wherein the one or more linkers comprise or consist of an amino acid sequence according to SEQ ID NO: 282.

Embodiment 133. The polyribonucleotide of any one of embodiments 111-132, wherein the polypeptide comprises a linker between the C-terminal region or portion thereof and the transmembrane region.

Embodiment 134. The polyribonucleotide of any one of embodiments 3-133, wherein the polypeptide comprises a linker after an amino acid sequence of NANPNVDP.

Embodiment 135. The polyribonucleotide of embodiment 1, wherein the polypeptide comprises:
(i) one or more Plasmodium CSP junction regions, portions thereof, or variants thereof according to any one of embodiments 25-45,
(ii) one or more repeats of the amino acid sequence of NANPNVDP according to any one of embodiments 4, 7-14 and 16,
(iii) one or more Plasmodium CSP C-terminal regions or portions thereof according to any one of embodiments 17-22,
(iv) a secretory signal according to any one of embodiments 95-110, and
(v) a transmembrane region according to any one of embodiments 111-125, and
wherein the polypeptide does not comprise:
(a) an amino acid sequence of NPNA, and
(b) a Plasmodium CSP N-terminal region or portion thereof.

Embodiment 136. The polyribonucleotide of embodiment 135, wherein the polypeptide does not comprise a Plasmodium CSP N-terminal end region.

Embodiment 137. The polyribonucleotide of embodiment 135, wherein the polypeptide comprises one or more Plasmodium CSP N-terminal end regions or portions thereof according to embodiments 46-48.

Embodiment 138. The polyribonucleotide of embodiment 1, wherein the polypeptide comprises:
(i) one or more Plasmodium CSP junction regions, portions thereof, or variants thereof according to any one of embodiments 25-45,
(ii) one or more repeats of the amino acid sequence of NANPNVDP according to embodiment 4, 7-9, 11-12, and 14-15,
(iii) one or more Plasmodium CSP C-terminal regions or portions thereof according to any one of embodiments 17-22, and
(iv) a secretory signal according to any one of embodiments 95-110.

Embodiment 139. The polyribonucleotide of embodiment 1, wherein the polypeptide comprises:
(i) three or more Plasmodium CSP junction regions, portions thereof, or variants thereof according to any one of embodiments 25-45,
(ii) three or more repeats of the amino acid sequence of NANPNVDP according to any one of embodiments 4, 7-9, 11-12, and 14-15,
(iii) two or more Plasmodium CSP major repeat region portions according to embodiments 56 or 57, and
(iv) a secretory signal according to any one of embodiments 95-110, and wherein the polypeptide does not comprise:
(a) a Plasmodium CSP N-terminal region or portion thereof, and
(b) a Plasmodium CSP C-terminal region or portion thereof.

Embodiment 140. The polyribonucleotide of embodiment 138 or 139, wherein the polypeptide comprises an amino acid sequence that has at least 85%, at least 90%, at least 95%, at least 96%, at least 97%, at least 98%, or at least 99%, or 100% sequence identity to a sequence of SEQ ID NO: 433.

Embodiment 141. The polyribonucleotide of embodiment 1, wherein the polypeptide comprises:
(i) one or more Plasmodium CSP junction regions, portions thereof, or variants thereof according to any one of embodiments 25-45,
(ii) one or more repeats of the amino acid sequence of NANPNVDP according to embodiment 4, 7-9, 11-12, and 14-15,
(iii) one or more Plasmodium CSP C-terminal regions or portions thereof according to any one of embodiments 17-22,
(iv) a secretory signal according to any one of embodiments 95-110; and
(v) a transmembrane region according to any one of embodiments 111-114.

Embodiment 142. The polyribonucleotide of embodiment 1, wherein the polypeptide comprises:
(i) three or more Plasmodium CSP junction regions, portions thereof, or variants thereof according to any one of embodiments 25-45,
(ii) three or more repeats of the amino acid sequence of NANPNVDP according to any one of embodiments 4, 7-9, 11-12, and 14-15,
(iii) two or more Plasmodium CSP major repeat region portions according to embodiments 56 or 57,
(iv) a secretory signal according to any one of embodiments 95-110,
(v) a transmembrane region according to any one of embodiments 111-114, and wherein the polypeptide does not comprise
(a) a Plasmodium CSP N-terminal region or portion thereof, and
(b) a Plasmodium CSP C-terminal region or portion thereof.

Embodiment 143. The polyribonucleotide of embodiment 141 or 142, wherein the polypeptide comprises an amino acid sequence that has at least 85%, at least 90%, at least 95%, at least 96%, at least 97%, at least 98%, or at least 99%, or 100% sequence identity to a sequence of SEQ ID NO: 432 OR 434. Embodiment 144. The polyribonucleotide of embodiment 1, wherein the polypeptide comprises:
(i) three or more Plasmodium CSP junction regions, portions thereof, or variants thereof according to any one of embodiments 25-45,
(ii) three or more repeats of the amino acid sequence of NANPNVDP according to any one of embodiments 4, 7-9, 11-12, and 14-15,
(iii) two or more Plasmodium CSP major repeat region portions according to embodiments 56 or 57, and
(iv) a secretory signal according to any one of embodiments 95-110, and wherein the polypeptide does not comprise:
(a) a Plasmodium CSP N-terminal region or portion thereof, and
(b) a Plasmodium CSP C-terminal region or portion thereof.

Embodiment 145. The polyribonucleotide of embodiment 144, wherein the polypeptide comprises an amino acid sequence that has at least 85%, at least 90%, at least 95%, at least 96%, at least 97%, at least 98%, or at least 99%, or 100% sequence identity to a sequence of SEQ ID NO: 435 or 436.

Embodiment 146. The polyribonucleotide of any one of embodiments 135-137, wherein the polypeptide comprises one or more helper antigens according to embodiments 78-89.

Embodiment 147. The polyribonucleotide of embodiment 1, wherein polypeptide comprises:
(i) a secretory signal according to any one of embodiments 95 and 99-106,
(ii) a Plasmodium CSP N-terminal end region according to embodiment 46 or 48,
(iii) a Plasmodium CSP junction region according to any one of embodiments 25, 27, and 28, (iv) nine repeats of the amino acid sequence of NANPNVDP according to any one of embodiments 4, 7, 8, 9, 11, 12, and 13,
(v) a Plasmodium CSP C-terminal region according to any one of embodiments 17, 18, and 20-22, and
(vi) five antigenic repeat regions, wherein each antigenic repeat region comprises:
   (A) a linker according to any one of embodiments 128-132, and
   (B) a helper antigen according to any one of embodiments 78-82, and
wherein the polypeptide does not comprise any of:
(a) an amino acid sequence of NPNA,
(b) a Plasmodium CSP N-terminal region or portion thereof, and
(c) a transmembrane region.

Embodiment 148. The polyribonucleotide of embodiment 147, wherein the polypeptide comprises or consists of an amino acid sequence with at least 85%, at least 90%, at least 95%, at least 96%, at least 97%, at least 98%, or at least 99%, or 100% sequence identity to an amino acid sequence according to SEQ ID NO: 36.

Embodiment 149. The polyribonucleotide of embodiment 1, wherein polypeptide comprises:
(i) a secretory signal according to any one of embodiments 95 and 99-106,
(ii) a helper antigen according to any one of embodiments 78-80, 83, and 84,
(iii) a linker according to any one of embodiments 128-132,
(iv) a Plasmodium CSP N-terminal end region according to embodiment 46 or 48,
(v) a Plasmodium CSP junction region according to any one of embodiments 25, 27, and 28,
(vi) nine repeats of the amino acid sequence of NANPNVDP according to any one of embodiments 4, 7, 8, 9, 11, 12, and 13,
(vii) a Plasmodium CSP C-terminal region according to any one of embodiments 17, 18, and 20-22,
(viii) a serine-valine sequence immediately following the Plasmodium CSP C-terminal region according to embodiment 24,
(ix) a linker according to any one of embodiments 128-132, and
(x) a transmembrane region according to any one of embodiments 111, and 115-122, and
wherein the polypeptide does not comprise any of:
(a) an amino acid sequence of NPNA, and
(b) a Plasmodium CSP N-terminal region or portion thereof.

Embodiment 150. The polyribonucleotide of embodiment 149, wherein the polypeptide comprises or consists of an amino acid sequence with at least 85%, at least 90%, at least 95%, at least 96%, at least 97%, at least 98%, or at least 99%, or 100% sequence identity to an amino acid sequence according to SEQ ID NO: 39.

Embodiment 151. The polyribonucleotide of embodiment 1, wherein polypeptide comprises:
(i) a secretory signal according to any one of embodiments 95 and 99-106,
(ii) a portion of a Plasmodium CSP junction region according to any one of embodiments 29-31, and 33,
(iii) three repeats of the amino acid sequence of NANPNVDP according to any one of embodiments 4, and 7-10,
(iv) a Plasmodium CSP C-terminal region according to any one of embodiments 17, 18, and 20-22,
(v) a serine-valine sequence immediately following the Plasmodium CSP C-terminal region according to embodiment 24,
(vi) a linker according to any one of embodiments 128-132, and
(vii) a transmembrane region according to any one of embodiments 111, and 115-122, and
wherein the polypeptide does not comprise any of:
(a) a Plasmodium CSP N-terminal region or portion thereof,
(b) a Plasmodium CSP N-terminal end region or portion thereof, and
(c) an amino acid sequence of NPNA.

Embodiment 152. The polyribonucleotide of embodiment 151, wherein the polypeptide comprises or consists of an amino acid sequence with at least 85%, at least 90%, at least 95%, at least 96%, at least 97%, at least 98%, or at least 99%, or 100% sequence identity to an amino acid sequence according to SEQ ID NO: 57.

Embodiment 153. The polyribonucleotide of embodiment 1, wherein polypeptide comprises:
(i) a secretory signal according to any one of embodiments 95 and 99-106,
(ii) a portion of a Plasmodium CSP junction region according to any one of embodiments 29-31, and 33,
(iii) nine repeats of the amino acid sequence of NANPNVDP according to any one of embodiments 4, 7, 8, 9, 11, 12, and 13,
(iv) a Plasmodium CSP C-terminal region according to any one of embodiments 17, 18, and 20-22,
(v) a serine-valine sequence immediately following the Plasmodium CSP C-terminal region according to embodiment 24,
(vi) a linker according to any one of embodiments 128-132, and
(vii) a transmembrane region according to any one of embodiments 111, and 115-122, and
wherein the polypeptide does not comprise any of:
(a) a Plasmodium CSP N-terminal region or portion thereof,
(b) a Plasmodium CSP N-terminal end region or portion thereof, and
(c) an amino acid sequence of NPNA.

Embodiment 154. The polyribonucleotide of embodiment 153, wherein the polypeptide comprises or consists of an amino acid sequence with at least 85%, at least 90%, at least 95%, at least 96%, at least 97%, at least 98%, or at least 99%, or 100% sequence identity to an amino acid sequence according to SEQ ID NO: 60.

Embodiment 155. The polyribonucleotide of embodiment 1, wherein polypeptide comprises:
(i) a secretory signal according to any one of embodiments 95 and 99-106,
(ii) a Plasmodium CSP junction region according to any one of embodiments 25, 27, and 28, (iii) three repeats of the amino acid sequence of NANPNVDP according to any one of embodiments 4, and 7-10,
(iv) a Plasmodium CSP C-terminal region according to any one of embodiments 17, 18, and 20-22,
(v) a serine-valine sequence immediately following the Plasmodium CSP C-terminal region according to embodiment 24,
(vi) a linker according to any one of embodiments 128-132, and
(vii) a transmembrane region according to any one of embodiments 111, and 115-122, and
wherein the polypeptide does not comprise any of:
(a) a Plasmodium CSP N-terminal region or portion thereof,
(b) a Plasmodium CSP N-terminal end region or portion thereof, and
(c) an amino acid sequence of NPNA.

Embodiment 156. The polyribonucleotide of embodiment 155, wherein the polypeptide comprises or consists of an amino acid sequence with at least 85%, at least 90%, at least 95%, at least 96%, at least 97%, at least 98%, or at least 99%, or 100% sequence identity to an amino acid sequence according to SEQ ID NO: 63.

Embodiment 157. The polyribonucleotide of embodiment 1, wherein the polypeptide comprises:
(i) a secretory signal according to any one of embodiments 95 and 99-106,
(ii) a Plasmodium CSP junction region according to any one of embodiments 25, 27, and 28,
(iii) nine repeats of the amino acid sequence of NANPNVDP according to any one of embodiments 4, 7, 8, 9, 11, 12, and 13,
(iv) a Plasmodium CSP C-terminal region according to any one of embodiments 17, 18 and 20-22,
(v) a serine-valine sequence immediately following the Plasmodium CSP C-terminal region according to embodiment 24,
(vi) a linker according to any one of embodiments 128-132, and
(vii) a transmembrane region according to any one of embodiments 111, and 115-122, and
wherein the polypeptide does not comprise any of:
(a) a Plasmodium CSP N-terminal region or portion thereof,
(b) a Plasmodium CSP N-terminal end region or portion thereof, and
(c) an amino acid sequence of NPNA.

Embodiment 158. The polyribonucleotide of embodiment 157, wherein the polypeptide comprises or consists of an amino acid sequence with at least 85%, at least 90%, at least 95%, at least 96%, at least 97%, at least 98%, or at least 99%, or 100% sequence identity to an amino acid sequence according to SEQ ID NO: 66.

Embodiment 159. The polyribonucleotide of embodiment 1, wherein polypeptide comprises:
(i) a secretory signal according to any one of embodiments 95 and 99-106,
(ii) a Plasmodium CSP junction region variant according to any one of embodiments 35-38, (iii) three repeats of the amino acid sequence of NANPNVDP according to any one of embodiments 4, and 7-10,
(iv) a Plasmodium CSP C-terminal region according to any one of embodiments 17, 18, and 20-22,
(v) a serine-valine sequence immediately following the Plasmodium CSP C-terminal region according to embodiment 24,
(vi) a linker according to any one of embodiments 128-132, and
(vii) a transmembrane region according to any one of embodiments 111, and 115-122, and
wherein the polypeptide does not comprise any of:
(a) a Plasmodium CSP N-terminal region or portion thereof,
(b) a Plasmodium CSP N-terminal end region or portion thereof, and
(c) an amino acid sequence of NPNA.

Embodiment 160. The polyribonucleotide of embodiment 159, wherein the polypeptide comprises or consists of an amino acid sequence with at least 85%, at least 90%, at least 95%, at least 96%, at least 97%, at least 98%, or at least 99%, or 100% sequence identity to an amino acid sequence according to SEQ ID NO: 69.

Embodiment 161. The polyribonucleotide of embodiment 1, wherein polypeptide comprises:
(i) a secretory signal according to any one of embodiments 95 and 99-106,
(ii) a Plasmodium CSP junction region variant according to any one of embodiments 42-45,
(iii) nine repeats of the amino acid sequence of NANPNVDP according to any one of embodiments 4, 7, 8, 9, 11, 12, and 13,
(iv) a Plasmodium CSP C-terminal region according to any one of embodiments 17, 18, and 20-22,
(v) a serine-valine sequence immediately following the Plasmodium CSP C-terminal region according to embodiment 24,
(vi) a linker according to any one of embodiments 128-132, and
(vii) a transmembrane region according to any one of embodiments 111, and 115-122, and
wherein the polypeptide does not comprise any of:
(a) a Plasmodium CSP N-terminal region or portion thereof,
(b) a Plasmodium CSP N-terminal end region or portion thereof, and
(c) an amino acid sequence of NPNA.

Embodiment 162. The polyribonucleotide of embodiment 161, wherein the polypeptide comprises or consists of an amino acid sequence with at least 85%, at least 90%, at least 95%, at least 96%, at least 97%, at least 98%, or at least 99%, or 100% sequence identity to an amino acid sequence according to SEQ ID NO: 72.

Embodiment 163. The polyribonucleotide of embodiment 1, wherein polypeptide comprises:
(i) a secretory signal according to any one of embodiments 95 and 99-106,
(ii) a portion of a Plasmodium CSP junction region according to any one of embodiments 29-32, and 34,
(iii) three repeats of the amino acid sequence of NANPNVDP according to any one of embodiments 4, and 7-10,
(iv) a Plasmodium CSP C-terminal region according to any one of embodiments 17, 18, and 20-22,
(v) a serine-valine sequence immediately following the Plasmodium CSP C-terminal region according to embodiment 24,
(vi) a linker according to any one of embodiments 128-132, and
(vii) a transmembrane region according to any one of embodiments 111, and 115-122, and
wherein the polypeptide does not comprise any of:
(a) a Plasmodium CSP N-terminal region or portion thereof,
(b) a Plasmodium CSP N-terminal end region or portion thereof, and
(c) an amino acid sequence of NPNA.

Embodiment 164. The polyribonucleotide of embodiment 163, wherein the polypeptide comprises or consists of an amino acid sequence with at least 85%, at least 90%, at least 95%, at least 96%, at least 97%, at least 98%, or at least 99%, or 100% sequence identity to an amino acid sequence according to SEQ ID NO: 75.

Embodiment 165. The polyribonucleotide of embodiment 1, wherein the polypeptide comprises:
(i) a secretory signal according to any one of embodiments 95 and 99-106,
(ii) a portion of a Plasmodium CSP junction region according to any one of embodiments 29-32, and 34,
(iii) nine repeats of the amino acid sequence of NANPNVDP according to any one of embodiments 4, 7, 8, 9, 11, 12, and 13,
(iv) a Plasmodium CSP C-terminal region according to any one of embodiments 17, 18, and 20-22,
(v) a serine-valine sequence immediately following the Plasmodium CSP C-terminal region according to embodiment 24,
(vi) a linker according to any one of embodiments 128-132, and
(vii) a transmembrane region according to any one of embodiments 111, and 115-122, and
wherein the polypeptide does not comprise any of:
(a) a Plasmodium CSP N-terminal region or portion thereof,
(b) a Plasmodium CSP N-terminal end region or portion thereof, and
(c) an amino acid sequence of NPNA.

Embodiment 166. The polyribonucleotide of embodiment 165, wherein the polypeptide comprises or consists of an amino acid sequence with at least 85%, at least 90%, at least 95%, at least 96%, at least 97%, at least 98%, or at least 99%, or 100% sequence identity to an amino acid sequence according to SEQ ID NO: 78.

Embodiment 167. The polyribonucleotide of embodiment 1, wherein polypeptide comprises:
(i) a secretory signal according to any one of embodiments 95 and 99-106,
(ii) a Plasmodium CSP N-terminal end region according to embodiment 46 or 48,
(iii) a Plasmodium CSP junction region according to any one of embodiments 25, 27, and 28,
(iv) nine repeats of the amino acid sequence of NANPNVDP according to any one of embodiments 4, 7, 8, 9, 11, 12, and 13,
(v) a Plasmodium CSP C-terminal region according to any one of embodiments 17, 18, and 20-22,
(vi) a serine-valine sequence immediately following the Plasmodium CSP C-terminal region according to embodiment 24,
(vii) a linker according to any one of embodiments 128-132, and
(viii) a transmembrane region according to any one of embodiments 111, and 115-122, and
wherein the polypeptide does not comprise any of:
(a) a Plasmodium CSP N-terminal region or portion thereof, and
(b) an amino acid sequence of NPNA.

Embodiment 168. The polyribonucleotide of embodiment 167, wherein the polypeptide comprises or consists of an amino acid sequence with at least 85%, at least 90%, at least 95%, at least 96%, at least 97%, at least 98%, or at least 99%, or 100% sequence identity to an amino acid sequence according to SEQ ID NO: 81.

Embodiment 169. The polyribonucleotide of embodiment 1, wherein polypeptide comprises:
(i) a secretory signal according to any one of embodiments 95 and 99-106,
(ii) a Plasmodium CSP N-terminal end region according to embodiment 46 or 48,
(iii) a Plasmodium CSP junction region variant according to any one of embodiments 42-45,
(iv) nine repeats of the amino acid sequence of NANPNVDP according to any one of embodiments 4, 7, 8, 9, 11, 12, and 13,
(v) a Plasmodium CSP C-terminal region according to any one of embodiments 17, 18, and 20-22,
(vi) a serine-valine sequence immediately following the Plasmodium CSP C-terminal region according to embodiment 24,
(vii) a linker according to any one of embodiments 128-132, and
(viii) a transmembrane region according to any one of embodiments 111 and 115-122, and
wherein the polypeptide does not comprise any of:
(a) a Plasmodium CSP N-terminal region or portion thereof, and
(b) an amino acid sequence of NPNA.

Embodiment 170. The polyribonucleotide of embodiment 169, wherein the polypeptide comprises or consists of an amino acid sequence with at least 85%, at least 90%, at least 95%, at least 96%, at least 97%, at least 98%, or at least 99%, or 100% sequence identity to an amino acid sequence according to SEQ ID NO: 84.

Embodiment 171. The polyribonucleotide of embodiment 1, wherein polypeptide comprises:
(i) a secretory signal according to any one of embodiments 95 and 99-106,
(ii) a Plasmodium CSP N-terminal end region according to embodiment 46 or 48,
(iii) a Plasmodium CSP junction region according to any one of embodiments 25, 27, and 28,
(iv) nine repeats of the amino acid sequence of NANPNVDP according to any one of embodiments 4, 7, 8, 9, 11, 12, and 13,
(v) a Plasmodium CSP C-terminal region according to any one of embodiments 17, 18, and 20-22,
(vi) a transmembrane region according to any one of embodiments 111-114, and
wherein the polypeptide does not comprise any of:
(a) a Plasmodium CSP N-terminal region or portion thereof, and
(b) an amino acid sequence of NPNA.

Embodiment 172. The polyribonucleotide of embodiment 171, wherein the polypeptide comprises or consists of an amino acid sequence with at least 85%, at least 90%, at least 95%, at least 96%, at least 97%, at least 98%, or at least 99%, or 100% sequence identity to an amino acid sequence according to SEQ ID NO: 96.

Embodiment 173. The polyribonucleotide of embodiment 1, wherein polypeptide comprises:
(i) a secretory signal according to any one of embodiments 95-98,
(ii) a Plasmodium CSP N-terminal end region according to embodiment 46 or 48,
(iii) a Plasmodium CSP junction region according to any one of embodiments 25, 27, and 28,
(iv) nine repeats of the amino acid sequence of NANPNVDP according to any one of embodiments 4, 7, 8, 9, 11, 12, and 13,
(v) a Plasmodium CSP C-terminal region according to any one of embodiments 17, 18, and 20-22, and
(vi) a transmembrane region according to any one of embodiments 111-114, and
wherein the polypeptide does not comprise any of:
(a) a Plasmodium CSP N-terminal region or portion thereof, and
(b) an amino acid sequence of NPNA.

Embodiment 174. The polyribonucleotide of embodiment 173, wherein the polypeptide comprises or consists of an amino acid sequence with at least 85%, at least 90%, at least 95%, at least 96%, at least 97%, at least 98%, or at least 99%, or 100% sequence identity to an amino acid sequence according to SEQ ID NO: 99.

Embodiment 175. The polyribonucleotide of embodiment 1, wherein polypeptide comprises:
(i) a secretory signal according to any one of embodiments 95 and 99-106,
(ii) two or more Plasmodium CSP neutralizing region repeats, wherein each Plasmodium CSP neutralizing region repeat comprises or consists of:
   (a) a Plasmodium CSP N-terminal end region according to embodiment 46 or 48,
   (b) a Plasmodium CSP junction region according to any one of embodiments 25, 27, and 28,
   (c) two repeats of the amino acid sequence of NANPNVDP according to any one of embodiments 7-9, and
   (d) a linker according to any one of embodiments 128-132,
(iii) a portion of a Plasmodium CSP major repeat region according to any one of embodiments 55, 58, and 62,
(iv) a Plasmodium CSP C-terminal region according to any one of embodiments 17, 18, and 20-22,
(v) a serine-valine sequence immediately following the Plasmodium CSP C-terminal region according to embodiment 24,
(vi) a linker according to any one of embodiments 128-132, and
(vii) a transmembrane region according to any one of embodiments 111, and 115-122, and
wherein the polypeptide does not comprise a Plasmodium CSP N-terminal region or portion thereof.

Embodiment 176. The polyribonucleotide of embodiment 175, wherein the polypeptide comprises exactly four Plasmodium CSP neutralizing region repeats.

Embodiment 177. The polyribonucleotide of embodiment 175 or 176, wherein the polypeptide comprises or consists of an amino acid sequence with at least 85%, at least 90%, at least 95%, at least 96%, at least 97%, at least 98%, or at least 99%, or 100% sequence identity to an amino acid sequence according to SEQ ID NO: 87.

Embodiment 178. The polyribonucleotide of embodiment 1, wherein the polypeptide comprises:
(i) a secretory signal according to any one of embodiments 95 and 99-106,
(ii) one Plasmodium CSP junction region according to any one of embodiments 25, 27, and 28,
(iii) three repeats of the amino acid sequence of NANPNVDP according to any one of embodiments 4, and 7-10,
(iv) a Plasmodium CSP major repeat region according to any one of embodiments 55-60 and 63,
(v) one Plasmodium CSP C-terminal region according to any one of embodiments 17, 18, and 20-22,
(vi) a serine-valine sequence immediately following the Plasmodium CSP C-terminal region according to embodiment 24,
(vii) a linker according to any one of embodiments 128-132, and
(viii) a transmembrane region according to any one of embodiments 111 and 115-122, and
wherein the polypeptide does not comprise any of:
(a) a Plasmodium CSP N-terminal region or portion thereof, and
(b) a Plasmodium CSP N-terminal end region or portion thereof.

Embodiment 179. The polyribonucleotide of embodiment 178, wherein the polypeptide comprises or consists of an amino acid sequence with at least 85%, at least 90%, at least 95%, at least 96%, at least 97%, at least 98%, or at least 99%, or 100% sequence identity to an amino acid sequence according to SEQ ID NO: 30.

Embodiment 180. The polyribonucleotide of embodiment 1, wherein polypeptide comprises:
(i) a secretory signal according to any one of embodiments 95-98,
(ii) a Plasmodium CSP N-terminal region according to any one of embodiments 50 and 52,
(iii) a Plasmodium CSP N-terminal end region according to embodiment 46 or 48,
(iv) a portion of a Plasmodium CSP junction region according to any one of embodiments 29-32, and 34,
(v) three repeats of the amino acid sequence of NANPNVDP according to any one of embodiments 4, and 7-10,
(vi) a Plasmodium CSP major repeat region according to any one of embodiments 54-60 and 63,
(vii) a Plasmodium CSP C-terminal region according to any one of embodiments 17, 18, and 20-22, and
(viii) a serine immediately following the Plasmodium CSP C-terminal region according to embodiment 23, and
   wherein the polypeptide does not comprise a transmembrane region.

Embodiment 181. The polyribonucleotide of embodiment 180, wherein the polypeptide comprises or consists of an amino acid sequence with at least 85%, at least 90%, at least 95%, at least 96%, at least 97%, at least 98%, or at least 99%, or 100% sequence identity to an amino acid sequence according to SEQ ID NO: 27.

Embodiment 182. The polyribonucleotide of embodiment 1, wherein polypeptide comprises:
(i) a secretory signal according to any one of embodiments 95-98,
(ii) a Plasmodium CSP N-terminal region according to any one of embodiments 50 and 52,
(iii) a Plasmodium CSP N-terminal end region according to embodiment 46 or 48,
(iv) a Plasmodium CSP junction region according to any one of embodiments 25, 27, and 28,
(v) three repeats of the amino acid sequence of NANPNVDP according to any one of embodiments 4, and 7-10,
(vi) a Plasmodium CSP major repeat region according to any one of embodiments 54-60 and 63,
(vii) a Plasmodium CSP C-terminal region according to any one of embodiments 17, 18, and 20-22, and
(viii) a serine immediately following the Plasmodium CSP C-terminal region according to embodiment 23, and
wherein the polypeptide does not comprise a transmembrane region.

Embodiment 183. The polyribonucleotide of embodiment 182, wherein the polypeptide comprises or consists of an amino acid sequence with at least 85%, at least 90%, at least 95%, at least 96%, at least 97%, at least 98%, or at least 99%, or 100% sequence identity to an amino acid sequence according to SEQ ID NO: 6.

Embodiment 184. The polyribonucleotide of embodiment 1, wherein the polypeptide comprises:
(i) a secretory signal according to any one of embodiments 95 and 107-110,
(ii) a Plasmodium CSP N-terminal region according to any one of embodiments 50 and 52,
(iii) a Plasmodium CSP N-terminal end region according to embodiment 46 or 48,
(iv) a Plasmodium CSP junction region according to any one of embodiments 25, 27, and 28,
(v) three repeats of the amino acid sequence of NANPNVDP according to any one of embodiments 4, and 7-10,
(vi) a Plasmodium CSP major repeat region according to any one of embodiments 54-60 and 63,
(vii) a Plasmodium CSP C-terminal region according to any one of embodiments 17, 18, and 20-22, and
(viii) a serine immediately following the Plasmodium CSP C-terminal region according to embodiment 23, and
wherein the polypeptide does not comprise a transmembrane region.

Embodiment 185. The polyribonucleotide of embodiment 184, wherein the polypeptide comprises or consists of an amino acid sequence with at least 85%, at least 90%, at least 95%, at least 96%, at least 97%, at least 98%, or at least 99%, or 100% sequence identity to an amino acid sequence according to SEQ ID NO: 24.

Embodiment 186. The polyribonucleotide of embodiment 1, wherein polypeptide comprises:
(i) a secretory signal according to any one of embodiments 95 and 99-106,
(ii) a Plasmodium CSP N-terminal region according to any one of embodiments 50 and 52,
(iii) a Plasmodium CSP N-terminal end region according to embodiment 46 or 48,
(iv) a Plasmodium CSP junction region according to any one of embodiments 25, 27, and 28,
(v) three repeats of the amino acid sequence of NANPNVDP according to any one of embodiments 4, and 7-10,
(vi) a Plasmodium CSP major repeat region according to any one of embodiments 54-60 and 63,
(vii) a Plasmodium CSP C-terminal region according to any one of embodiments 17, 18, and 20-22, and
(v) a serine-valine sequence immediately following the Plasmodium CSP C-terminal region according to embodiment 24, and
wherein the polypeptide does not comprise a transmembrane region.

Embodiment 187. The polyribonucleotide of embodiment 186, wherein the polypeptide comprises or consists of an amino acid sequence with at least 85%, at least 90%, at least 95%, at least 96%, at least 97%, at least 98%, or at least 99%, or 100% sequence identity to an amino acid sequence according to SEQ ID NO: 93.

Embodiment 188. The polyribonucleotide of embodiment 1, wherein the polypeptide comprises:
(i) a secretory signal according to any one of embodiments 95-98,
(ii) a Plasmodium CSP N-terminal region according to any one of embodiments 50 and 52,
(iii) a Plasmodium CSP N-terminal end region according to embodiment 46 or 48,
(iv) a Plasmodium CSP junction region according to any one of embodiments 25, 27, and 28,
(v) three repeats of the amino acid sequence of NANPNVDP according to any one of embodiments 4, and 7-10,
(vi) a Plasmodium CSP major repeat region according to any one of embodiments 54-60 and 63,
(vii) a Plasmodium CSP C-terminal region according to any one of embodiments 17, 18, and 20-22, and
(viii) a transmembrane region according to any one of embodiments 111-114.

Embodiment 189. The polyribonucleotide of embodiment 188, wherein the polypeptide comprises or consists of an amino acid sequence with at least 85%, at least 90%, at least 95%, at least 96%, at least 97%, at least 98%, or at least 99%, or 100% sequence identity to an amino acid sequence according to SEQ ID NO: 33.

Embodiment 190. The polyribonucleotide of embodiment 1, wherein the polypeptide comprises:
(i) a secretory signal according to any one of embodiments 95 and 99-106,
(ii) a Plasmodium CSP N-terminal region according to any one of embodiments 50 and 52,
(iii) a Plasmodium CSP N-terminal end region according to embodiment 46 or 48,
(iv) a Plasmodium CSP junction region according to any one of embodiments 25, 27, and 28,
(v) three repeats of the amino acid sequence of NANPNVDP according to any one of embodiments 4, and 7-10,
(vi) a Plasmodium CSP major repeat region according to any one of embodiments 54-60 and 63,
(vii) a Plasmodium CSP C-terminal region according to any one of embodiments 17, 18, and 20-22,
(viii) a serine-valine sequence immediately following the Plasmodium CSP C-terminal region according to embodiment 24,
(ix) a linker according to any one of embodiments 128-132,
(x) a multimerization region according to any one of embodiments 90-94, and
wherein the polypeptide does not comprise a transmembrane region.

Embodiment 191. The polyribonucleotide of embodiment 190, wherein the polypeptide comprises or consists of an amino acid sequence with at least 85%, at least 90%, at least 95%, at least 96%, at least 97%, at least 98%, or at least 99%, or 100% sequence identity to an amino acid sequence according to SEQ ID NO: 42.

Embodiment 192. The polyribonucleotide of embodiment 1, wherein the polypeptide comprises:
(i) a secretory signal according to any one of embodiments 95-99,
(ii) a Plasmodium CSP N-terminal region according to any one of embodiments 50 and 52,
(iii) a Plasmodium CSP N-terminal end region according to embodiment 46 or 48,
(iv) a Plasmodium CSP junction region according to any one of embodiments 25, 27, and 28,
(v) three repeats of the amino acid sequence of NANPNVDP according to any one of embodiments 4, and 7-10,
(vi) a Plasmodium CSP major repeat region according to any one of embodiments 54-60 and 63,
(vii) a Plasmodium CSP C-terminal region according to any one of embodiments 17, 18, and 20-22,
(viii) a serine immediately following the Plasmodium CSP C-terminal region according to embodiment 23,
(ix) a linker according to any one of embodiments 128-132, and
(x) a transmembrane region according to any one of embodiments 111, 115, 116, and 123-125.

Embodiment 193. The polyribonucleotide of embodiment 192, wherein the polypeptide comprises or consists of an amino acid sequence with at least 85%, at least 90%, at least 95%, at least 96%, at least 97%, at least 98%, or at least 99%, or 100% sequence identity to an amino acid sequence according to SEQ ID NO: 48.

Embodiment 194. The polyribonucleotide of embodiment 1, wherein the polypeptide comprises:
(i) a secretory signal according to any one of embodiments 95 and 100-106,
(ii) a Plasmodium CSP N-terminal region according to any one of embodiments 50 and 52,
(iii) a Plasmodium CSP N-terminal end region according to embodiment 46 or 48,
(iv) a Plasmodium CSP junction region according to any one of embodiments 25, 27, and 28,
(v) three repeats of the amino acid sequence of NANPNVDP according to any one of embodiments 4, and 7-10,
(vi) a Plasmodium CSP major repeat region according to any one of embodiments 54-60 and 63,
(vii) a Plasmodium CSP C-terminal region according to any one of embodiments 17, 18, and 20-22,
(viii) a serine-valine sequence immediately following the Plasmodium CSP C-terminal region according to embodiment 24,
(ix) a linker according to any one of embodiments 128-132,
(x) a transmembrane region according to any one of embodiments 111 and 115-122.

Embodiment 195. The polyribonucleotide of embodiment 194, wherein the polypeptide comprises or consists of an amino acid sequence with at least 85%, at least 90%, at least 95%, at least 96%, at least 97%, at least 98%, or at least 99%, or 100% sequence identity to an amino acid sequence according to SEQ ID NO: 90.

Embodiment 196. The polyribonucleotide of embodiment 1, wherein polypeptide comprises:
(i) a secretory signal according to any one of embodiments 95 and 100-106,
(ii) a Plasmodium CSP N-terminal region according to any one of embodiments 50 and 52,
(iii) a Plasmodium CSP N-terminal end region according to embodiment 46 or 48,
(iv) a Plasmodium CSP junction region according to any one of embodiments 25, 27, and 28,
(v) three repeats of the amino acid sequence of NANPNVDP according to any one of embodiments 4, and 7-10,
(vi) a Plasmodium CSP major repeat region according to any one of embodiments 54-60 and 63,
(vii) a Plasmodium CSP C-terminal region according to any one of embodiments 17, 18, and 20-22,
(viii) a serine immediately following the Plasmodium CSP C-terminal region according to embodiment 23, and
(ix) a transmembrane region according to any one of embodiments 111 and 115-122.

Embodiment 197. The polyribonucleotide of embodiment 196, wherein the polypeptide comprises or consists of an amino acid sequence with at least 85%, at least 90%, at least 95%, at least 96%, at least 97%, at least 98%, or at least 99%, or 100% sequence identity to an amino acid sequence according to SEQ ID NO: 21.

Embodiment 198. The polyribonucleotide of embodiments 135-197, wherein, when present, the features (i) to (x) are in the polypeptide in numerical order from the C-terminus to the N-terminus.

Embodiment 199. The polyribonucleotide of any one of embodiments 1-198, wherein Plasmodium is Plasmodium falciparum.

Embodiment 200. The polyribonucleotide of any one of embodiments 1-198, wherein the one or more Plasmodium CSP polypeptide regions or portions thereof are one or more P. falciparum CSP polypeptide regions or portions thereof.

Embodiment 201. The polyribonucleotide of 199 or 200, wherein Plasmodium falciparum is Plasmodium falciparum isolate 3D7.

Embodiment 202. The polyribonucleotide of any one of embodiments 1-201, wherein the polyribonucleotide is an isolated polyribonucleotide.

Embodiment 203. The polyribonucleotide of any one of embodiments 1-202, wherein the polyribonucleotide is an engineered polyribonucleotide.

Embodiment 204. The polyribonucleotide of any one of embodiments 1-203, wherein the polyribonucleotide is a codon-optimized polyribonucleotide.

Embodiment 205. An RNA construct comprising in 5′ to 3′ order:
(i) a 5′ UTR that comprises or consists of a modified human alpha-globin 5′-UTR;
(ii) a polyribonucleotide of any one of embodiments 1-204;
(iii) a 3′ UTR that comprises or consists of a first sequence from the amino terminal enhancer of split (AES) messenger RNA and a second sequence from the mitochondrial encoded 12S ribosomal RNA; and
(iv) a polyA tail sequence.

Embodiment 206. The RNA construct of embodiment 205, wherein the 5′ UTR comprises or consists of a ribonucleic acid sequence according to SEQ ID NO: 415.

Embodiment 207. The RNA construct of embodiment 205 or 206, wherein the 3′ UTR comprises or consists of a ribonucleic acid sequence according to SEQ ID NO: 416.

Embodiment 208. The RNA construct of any one of embodiments 205-207, wherein the polyA tail sequence is a split polyA tail sequence.

Embodiment 209. The RNA construct of embodiment 208, wherein the split polyA tail sequence comprises or consists of a ribonucleic acid sequence according to SEQ ID NO: 417.

Embodiment 210. The RNA construct of any one of embodiments 205-209, further comprising a 5′ cap.

Embodiment 211. The RNA construct of any one of embodiments 205-210, further comprising a cap proximal sequence comprising positions +1, +2, +3, +4, and +5 of the polyribonucleotide.

Embodiment 212. The RNA construct of embodiment 210 or 211, wherein the 5′ cap comprises or consists of a Cap1 structure comprising m7 (3′-OMeG) (5′) ppp (5′) (2′-OMeA₁) pG₂, wherein A1 is position +1 of the polyribonucleotide, and G₂ is position +2 of the polyribonucleotide.

Embodiment 213. The RNA construct of embodiment 212, wherein the cap proximal sequence comprises A1 and G₂ of the Cap1 structure, and a sequence comprising: A3A4Us (SEQ ID NO: 424) at positions +3, +4 and +5 respectively of the polyribonucleotide.

Embodiment 214. The RNA construct of any one of embodiments 205-213, wherein the polyribonucleotide includes modified uridines in place of all uridines.

Embodiment 215. The RNA construct of embodiment 214, wherein the modified uridines are each N₁-methyl-pseudouridine.

Embodiment 216. A composition comprising one or more polyribonucleotides of any one of embodiments 1-204.

Embodiment 217. A composition comprising one or more RNA constructs of any one of embodiments 205-214.

Embodiment 218. The composition of embodiment 216 or 217, wherein the composition further comprises lipid nanoparticles, polyplexes (PLX), lipidated polyplexes (LPLX), or liposomes, wherein the one or more polyribonucleotides or the one or more RNA constructs are fully or partially encapsulated within the lipid nanoparticles, polyplexes (PLX), lipidated polyplexes (LPLX), or liposomes.

Embodiment 219. The composition of any one of embodiments 216-218, wherein the composition further comprises lipid nanoparticles, wherein the one or more polyribonucleotides or the one or more RNA constructs are fully or partially encapsulated within the lipid nanoparticles.

Embodiment 220. The composition of embodiment 218 or 219, wherein the lipid nanoparticles target liver cells.

Embodiment 221. The composition of embodiment 218 or 219, wherein the lipid nanoparticles target secondary lymphoid organ cells.

Embodiment 222. The composition of any one of embodiments 218-221, wherein the lipid nanoparticles are cationic lipid nanoparticles.

Embodiment 223. The composition of any one of embodiments 218-222, wherein the lipid nanoparticles each comprise:
(a) a polymer-conjugated lipid;
(b) a cationically ionizable lipid; and
(c) one or more neutral lipids.

Embodiment 224. The composition of embodiment 223, wherein the polymer-conjugated lipid comprises a PEG-conjugated lipid.

Embodiment 225. The composition of embodiment 223 or 224, wherein the polymer-conjugated lipid comprises 2-[(polyethylene glycol)-2000]—N,N-ditetradecylacetamide.

Embodiment 226. The composition of any one of embodiments 223-225, wherein the one or more neutral lipids comprise 1,2-Distearoyl-sn-glycero-3-phosphocholine (DPSC).

Embodiment 227. The composition of any one of embodiments 223-226, wherein the one or more neutral lipids comprise cholesterol.

Embodiment 228. The composition of any one of embodiments 223-227, wherein the cationically ionizable lipid comprises [(4-Hydroxybutyl) azanediyl]di(hexane-6,1-diyl)bis(2-hexyldecanoate).

Embodiment 229. The composition of any one of embodiments 223-228, wherein the lipid nanoparticles have an average diameter of about 50-150 nm.

Embodiment 230. A pharmaceutical composition comprising the composition of any one of embodiments 216-229 and at least one pharmaceutically acceptable excipient.

Embodiment 231. The pharmaceutical composition of embodiment 230, wherein the pharmaceutical composition comprises a cryoprotectant, optionally wherein the cryoprotectant is sucrose.

Embodiment 232. The pharmaceutical composition of embodiment 230 or 231, wherein the pharmaceutical composition comprises an aqueous buffered solution, optionally wherein the aqueous buffered solution comprises one or more of Tris base, Tris HCl, NaCl, KCl, NazHPO4, and KH₂PO₄.

Embodiment 233. A combination comprising:
(i) a first pharmaceutical composition comprising a first polyribonucleotide, wherein the first polyribonucleotide encodes a first polypeptide, and the first polypeptide comprises one or more Plasmodium CSP polypeptide regions or portions thereof; and
(ii) a second pharmaceutical composition comprising a second polyribonucleotide, wherein the second polyribonucleotide encodes a second polypeptide, and the second polypeptide comprises one or more Plasmodium T-cell antigens.

Embodiment 234. The combination of embodiment 233, wherein the first polyribonucleotide is a polyribonucleotide according to any one of embodiments 1-204 or an RNA construct according to any one of embodiments 205-215.

Embodiment 235. A method comprising administering a polyribonucleotide according to any one of embodiments 1-204 to a subject.

Embodiment 236. A method comprising administering an RNA construct according to any one of embodiments 205-213 to a subject.

Embodiment 237. A method comprising administering a composition according to any one of embodiments 218-229 to a subject.

Embodiment 238. A method comprising administering one or more doses of the pharmaceutical composition of any one of embodiments 230-232 to a subject.

Embodiment 239. The pharmaceutical composition of any one of embodiments 230-232 for use in the treatment of a malaria infection comprising administering one or more doses of the pharmaceutical composition to a subject.

Embodiment 240. The pharmaceutical composition of any one of embodiments 230-232 for use in the prevention of a malaria infection comprising administering one or more doses of the pharmaceutical composition to a subject.

Embodiment 241. The method of embodiment 238 or the pharmaceutical composition for use of embodiment 239 or 240, comprising administering two or more doses of the pharmaceutical composition to a subject.

Embodiment 242. The method of embodiment 238 or 241, or the pharmaceutical composition for use of any one of embodiments 239-241, comprising administering three or more doses of the pharmaceutical composition to a subject.

Embodiment 243. The method or the pharmaceutical composition for use of embodiment 242, wherein the second of the three or more doses is administered to the subject at least 4 weeks after the first of the three or more doses is administered to the subject.

Embodiment 244. The method or the pharmaceutical composition for use of embodiment 242 or 243, wherein the third of the three or more doses is administered to the subject at least 4 weeks after the second of the three or more doses is administered to the subject.

Embodiment 245. The method of any one of embodiments 238 or 241-244, or the pharmaceutical composition for use of any one of embodiments 239-244, comprising administering a fourth dose of the pharmaceutical composition to a subject.

Embodiment 246. The method or the pharmaceutical composition for use of embodiment 242, wherein the fourth dose is administered to the subject at least 4 weeks after the third of the three or more doses is administered to the subject.

Embodiment 247. The method or the pharmaceutical composition for use of embodiment 245, wherein the fourth dose is administered to the subject at least one year after the third of the three or more doses is administered to the subject.

Embodiment 248. A method comprising administering a combination of embodiment 233 or 234 to a subject.

Embodiment 249. The method of embodiment 248, wherein the first pharmaceutical composition and the second pharmaceutical composition are administered on the same day.

Embodiment 250. The method of embodiment 248, wherein the first pharmaceutical composition and the second pharmaceutical composition are administered on different days.

Embodiment 251. The method of any one of embodiments 248-250, wherein the first pharmaceutical composition and the second pharmaceutical composition are administered to the subject at different locations on the subject's body.

Embodiment 252. The method of any one of embodiments 248-251, wherein the method is a method of treating a malaria infection.

Embodiment 253. The method of any one of embodiments 248-252, wherein the method is a method of preventing a malaria infection.

Embodiment 254. The method of any one of embodiments 248-253, wherein the subject has or is at risk of developing a malaria infection.

Embodiment 255. The method of any one of embodiments 248-254, wherein the subject is a human.

Embodiment 256. The method of any one of embodiments 235-238 and 241-255, wherein administration induces an anti-malaria immune response in the subject.

Embodiment 257. The method of embodiment 256, wherein the anti-malaria immune response in the subject comprises an adaptive immune response.

Embodiment 258. The method of embodiment 256 or 257, wherein the anti-malaria immune response in the subject comprises a T-cell response.

Embodiment 259. The method of embodiment 258, wherein the T-cell response is or comprises a CD4+ T cell response.

Embodiment 260. The method of embodiment 258 or 259, wherein the T-cell response is or comprises a CD8+ T cell response.

Embodiment 261. The method of any one of embodiments 256-260, wherein the anti-malaria immune response comprises a B-cell response.

Embodiment 262. The method of any one of embodiments 256-261, wherein the anti-malaria immune response comprises the production of antibodies directed against the one or more Plasmodium antigens.

Embodiment 263. Use of the pharmaceutical composition of any one of embodiments 230-232 in the treatment of a malaria infection.

Embodiment 264. Use of the pharmaceutical composition of any one of embodiments 230-232 in the prevention of a malaria infection.

Embodiment 265. Use of the pharmaceutical composition of any one of embodiments 230-232 in inducing an anti-malaria immune response in a subject.

Embodiment 266. A polypeptide encoded by a polyribonucleotide of any one of embodiments 1-204.

Embodiment 267. A polypeptide encoded by an RNA construct of any one of embodiments 205-215.

Embodiment 268. A host cell comprising a polyribonucleotide of any one of embodiments 1-204.

Embodiment 269. A host cell comprising an RNA construct of any one of embodiments 205-215. Embodiment 270. A host cell comprising a polypeptide of embodiment 266 or 267. [00922]

Embodiment 271. An RNA construct comprising in 5′ to 3′ order:
(i) a 5′ UTR that comprises or consists of a modified human alpha-globin 5′-UTR;
   (ii) a polyribonucleotide encoding a Plasmodium polypeptide having an amino acid sequence with at least 85% sequence identity with an amino acid sequence according to SEQ ID NO: 33 or 81;
   (iii) a 3′ UTR that comprises or consists of a first sequence from the amino terminal enhancer of split (AES) messenger RNA and a second sequence from the mitochondrial encoded 12S ribosomal RNA; and
   (iv) a polyA tail sequence.

Embodiment 272. An RNA construct comprising in 5′ to 3′ order:
(i) a 5′ UTR that comprises or consists of a modified human alpha-globin 5′-UTR;
(ii) a polyribonucleotide encoding a Plasmodium polypeptide comprising one or more Plasmodium CSP polypeptide regions or portions thereof, wherein the one or more Plasmodium CSP polypeptide regions or portions thereof comprise one or more repeats of the amino acid sequence of NANPNVDP, and wherein the Plasmodium polypeptide does not comprise the amino acid sequence of NPNA;
(iii) a 3′ UTR that comprises or consists of a first sequence from the amino terminal enhancer of split (AES) messenger RNA and a second sequence from the mitochondrial encoded 12S ribosomal RNA; and
(iv) a polyA tail sequence.

Embodiment 273. The RNA construct of embodiment 272, wherein the Plasmodium polypeptide comprises five or more repeats of the amino acid sequence of NANPNVDP.

Embodiment 274. The RNA construct of embodiment 272 or 273, wherein the Plasmodium polypeptide comprises a heterologous secretory signal.

Embodiment 275. The RNA construct of embodiment 274, wherein the heterologous secretory signal is an HSV secretory signal.

Embodiment 276. The RNA construct of embodiment 275, wherein the HSV secretory signal is an HSV gD secretory signal.

Embodiment 277. The RNA construct of any one of embodiments 272-276, wherein the Plasmodium polypeptide comprises a heterologous transmembrane domain.

Embodiment 278. The RNA construct of embodiment 277, wherein the heterologous transmembrane domain is an HSV transmembrane domain.

Embodiment 279. The RNA construct of embodiment 278, wherein the HSV transmembrane domain is an HSV gD transmembrane domain.

Embodiment 280. The RNA construct of any one of embodiments 272-279, wherein the Plasmodium polypeptide comprises:
(i) a heterologous secretory signal,
(ii) a Plasmodium CSP N-terminal end region,
(iii) a Plasmodium CSP junction region,
(iv) nine repeats of the amino acid sequence of NANPNVDP,
(v) a Plasmodium CSP C-terminal region,
(vi) a serine-valine sequence immediately following the Plasmodium CSP C-terminal region,
(vii) a linker, and
(viii) a heterologous transmembrane region;
wherein the polypeptide does not comprise any of:
   (a) a Plasmodium CSP N-terminal region or portion thereof, and
   (b) an amino acid sequence of NPNA.

Embodiment 281. The RNA construct of any one of embodiments 272-280, wherein the polypeptide comprises or consists of an amino acid sequence with at least 85% sequence identity to an amino acid sequence according to SEQ ID NO: 81.

Embodiment 282. The RNA construct of any one of embodiments 271-281, wherein the Plasmodium is Plasmodium falciparum.

Embodiment 283. The RNA construct of embodiment 282, wherein Plasmodium falciparum is Plasmodium falciparum isolate 3D7.

Embodiment 284. The RNA construct of any one of embodiments 271-283, wherein the polyribonucleotide is an isolated polyribonucleotide.

Embodiment 285. The RNA construct of any one of embodiments 271-284, wherein the polyribonucleotide is an engineered polyribonucleotide.

Embodiment 286. The RNA construct of any one of embodiments 271-285, wherein the polyribonucleotide is a codon-optimized polyribonucleotide.

Embodiment 287. The RNA construct of any one of embodiments 271-286, wherein the 5′ UTR comprises or consists of a ribonucleic acid sequence according to SEQ ID NO: 415.

Embodiment 288. The RNA construct of any one of embodiments 271-287, wherein the 3′ UTR comprises or consists of a ribonucleic acid sequence according to SEQ ID NO: 416.

Embodiment 289. The RNA construct of any one of embodiments 271-288, wherein the polyA tail sequence is a split polyA tail sequence.

Embodiment 290. The RNA construct of embodiment 289, wherein the split polyA tail sequence comprises or consists of a ribonucleic acid sequence according to SEQ ID NO: 417.

Embodiment 291. The RNA construct of any one of embodiments 271-290, further comprising a 5′ cap.

Embodiment 292. The RNA construct of any one of embodiments 271-291, further comprising a cap proximal sequence comprising positions +1, +2, +3, +4, and +5 of the polyribonucleotide.

Embodiment 293. The RNA construct of embodiment 291 or 292, wherein the 5′ cap comprises or consists of a Cap1 structure comprising m7 (3′-OMeG) (5′) ppp (5′) (2′-OMeA₁) pG₂, wherein A₁ is position +1 of the polyribonucleotide, and G₂ is position +2 of the polyribonucleotide.

Embodiment 294. The RNA construct of embodiment 292 or 293, wherein the cap proximal sequence comprises A₁ and G₂ of the Cap1 structure, and a sequence comprising: A₃A₄U₅ (SEQ ID NO: 424) at positions +3, +4 and +5 respectively of the polyribonucleotide.

Embodiment 295. The RNA construct of any one of embodiments 271-294, wherein the RNA construct includes modified uridines in place of one or more uridines.

Embodiment 296. The RNA construct of any one of embodiments 271-295, wherein the RNA construct includes modified uridines in place of all uridines.

Embodiment 297. The RNA construct of embodiment 295 or 296, wherein the modified uridines are each N₁-methyl-pseudouridine.

Embodiment 298. A composition comprising one or more RNA constructs of any one of embodiments 271-297.

Embodiment 299. The composition of embodiment 298, further comprising lipid nanoparticles, polyplexes (PLX), lipidated polyplexes (LPLX), or liposomes,
wherein the one or more RNA constructs are fully or partially encapsulated within the lipid nanoparticles, polyplexes (PLX), lipidated polyplexes (LPLX), or liposomes.

Embodiment 300. The composition of embodiment 298 or 299, further comprising lipid nanoparticles, wherein the one or more RNA constructs are fully or partially encapsulated within the lipid nanoparticles.

Embodiment 301. A pharmaceutical composition comprising the composition of any one of embodiments 298-300 and at least one pharmaceutically acceptable excipient.

Embodiment 302. A method of treating or preventing a malaria infection comprising administering to a subject an RNA construct according to any one of embodiments 271-297, a composition according to any one of embodiments 298-300, or pharmaceutical composition of embodiment 301.

Embodiment 303. The pharmaceutical composition of embodiment 301 for use in the treatment or prevention of a malaria infection comprising administering one or more doses of the pharmaceutical composition to a subject.

### EXEMPLIFICATION

### Example 1: In-Vitro Expression of Exemplary Polyribonucleotides Encoding Plasmodium Polypeptide Constructs

The present Example demonstrates that exemplary polyribonucleotides encoding different Plasmodium polypeptide constructs, as described herein, exhibit in-vitro expression (e.g., intracellular, surface) in mammalian cells (HEK293T cells).

In vitro expression assays were used to assess expression and localization of different Plasmodium polypeptide constructs. Polyribonucleotides encoding various Plasmodium polypeptide constructs depicted in Table 5 were generated, and the polyribonucleotides (“RNA constructs”) were numbered according to the corresponding encoded construct number shown in Table 5. As indicated with Table 5 above, as used herein, an “ERMA” construct is an “RNA construct,” and for example, “ERMA 1” corresponds to “RNA Construct 1,” “ERMA 2” corresponds to “RNA Construct 2,” etc. Assays were initially performed with non-formulated RNA constructs to determine functionality. Formulated RNA constructs were also assessed.

Briefly, HEK293T cells were transfected with (i) 350 ng of RNA constructs or (ii) 5 ng or 50 ng of LNP formulated RNA constructs. HEK293T cells transfected with 350 ng of RNA constructs or with 5 ng of formulated RNA constructs were assessed for protein expression by antibody staining and FACS. Transfection rate was determined by measuring percentage of positive cells, and total expression was determined by measuring median fluorescence of the total HEK population. HEK293T cells transfected with 50 ng of formulated RNA constructs were assessed for protein secretion by detecting protein in the culture supernatant of transfected cells.

### (1) Transfection of Mammalian Cells (e.g., HEK 293T Cells) in Multi-Well Plates

A sub confluent T175 flask (e.g., 80-90% confluency) of HEK293T cells was used for cell seeding by detaching cells using an enzyme mixture of proteolytic and collagenolytic enzyme activity (e.g., Accutase®). Detached cells were collected and 0.4×10\hat{ }5 cells were seeded in 1000 μl/well of a 12-well plate on the day of transfection.

For transfection of mammalian cells with non-formulated RNA, the use of a transfection agent is necessary, and, in this case, cell transfection was performed with MessengerMax Transfection Reagent following the manufacturers protocol. MessengerMax is diluted in OptiMEM so that there is 2 μl of the transfection agent per well, and this mixture was incubated at room temperature (RT) for 10 min. For each tested sample, 100 ng of RNA per well was diluted in 100 μl of OptiMEM and MessengerMax mix. The RNA-MessengerMax-Mix was incubated for 5 min at RT to allow complex formation. About 100 μl/well of the transfection mixture was added dropwise onto the cells. The transfected cells were then incubated overnight (e.g., 18 hours) at 37° C., 5% CO2 under humidified atmosphere.

For transfection of mammalian cells with formulated RNA, the formulated product was diluted (e.g., in a range of 5 ng-200 ng) in 100 μl OptiMEM per well and the mixture added dropwise on the cells. Culture plates were subsequently centrifuged at 300×g, 5 min at 21° C. and were then incubated overnight (e.g., 18 hours) at 37° C., 5% CO2 under humidified atmosphere.

### (2) Flow Cytometry Analysis for Detection of Protein Expression

The transfected cells were subject to flow cytometry analysis quantifying protein expression. Briefly, transfected cells were washed with DPBS and transferred to 96-well plate for staining. For cells transfected with RNA constructs 1, 2, 4-9, 23-41, 59, and 60, cells were stained firstly for viability (Fixable Viability Dye eFluor™ 450; 1:500), and then with a primary antibody against Pf PfCSP (either human anti-PfPfCSP L9 targeting minor repeats (1:60,000) or mouse anti-Pf PfCSP 2A10 targeting major repeats (1:2000)) and a fluorescent secondary antibody (either anti-human-AlexaFluor® 674 (1:1000) or anti-mouse-AlexaFluor® 647 (1:500)). For cells transfected with RNA constructs 59, 60, 87, 88, 91, 100, and 104, cells were stained with a human anti-CSP, clone L⁹, at a dilution 1:60,000 and then a fluorescent secondary antibody (anti-human-AlexaFluor® 674 (1:1000)). A permeabilization step was included prior to staining with antibodies. After staining, cells were resuspended in 180 μl FACS Buffer (DPBS with 1% BSA, 0.5 mM EDTA) and 75 μl of the cells was acquired for flow cytometry analysis using BD FACSCelesta Cell Analyzer.

Expression of constructs in HEK293T cells was evaluated using flow cytometry by measuring total protein in permeabilized HEK293T cells. For all, transfection rate illustrates the percentage of cells (% positive cells) that express protein encoded by the construct in question. Total expression shows the median fluorescence (MedianFI) of the total HEK293T population, indicating the amount of signal measured from the translated protein. All constructs were expressed in varying degrees in the in vitro model.

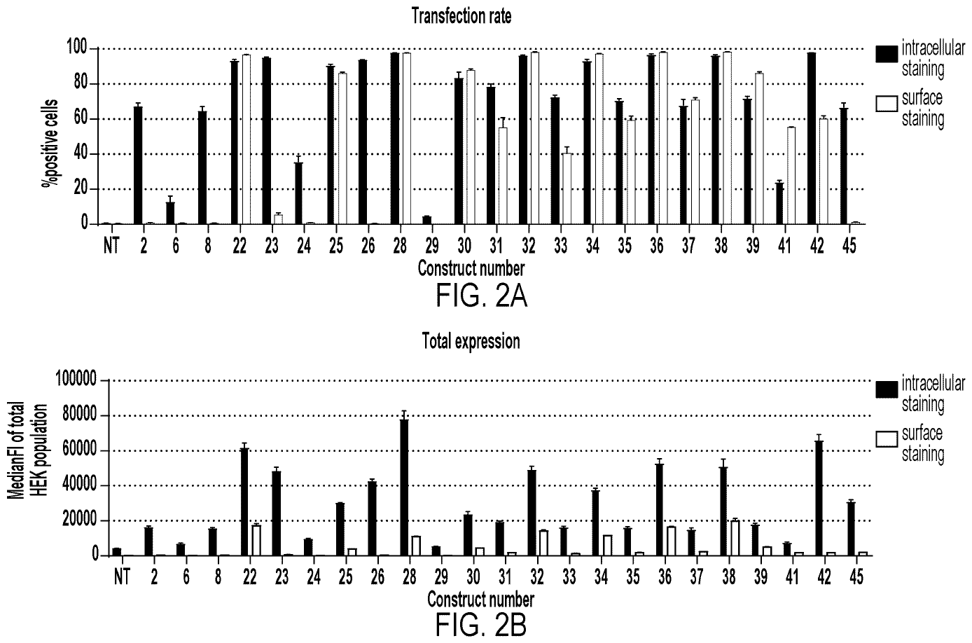

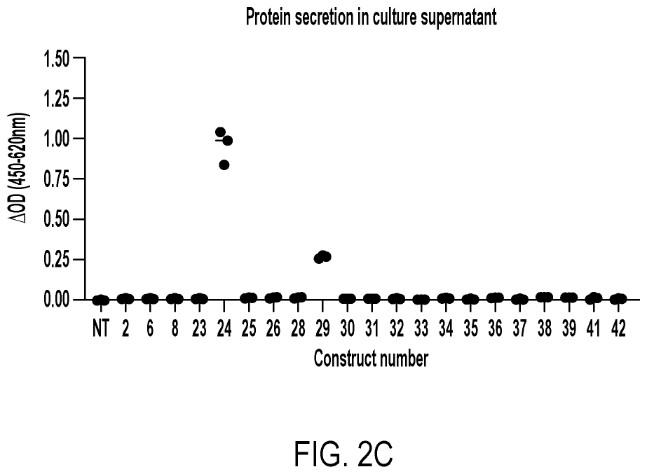

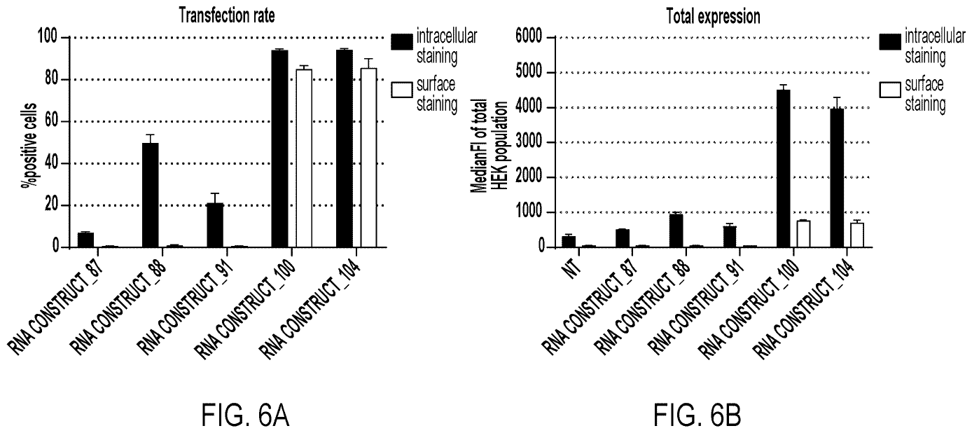

Both non-formulated (FIGS. 1, 3, 4 and 5) and formulated (FIGS. 2 and 6) RNA constructs had an overall high transfection rate. As shown in FIG. 1A, RNA constructs 7, 25, 28 and 30-40 had the highest transfection rates (at least about 70% positive cells). Furthermore, polyribonucleotides encoding Plasmodium polypeptide constructs with a TM domain or GPI anchor (RNA constructs 7, 23, 25, 28, and 30-41) were expressed on the surface, while those without (RNA constructs 1, 2, 4, 5, 6, 8, 9, 24, 26, 27, and 29) were not. As shown in FIG. 1B, RNA constructs 28, 36 and 37 had the highest expression overall, exhibiting intracellular staining with a median fluorescence intensity of at least about 150,000 and surface staining with a median fluorescence intensity of at least about 75,000.

As shown in FIG. 2A, all RNA constructs were expressed; however, some RNA constructs (22, 25, 26, 28, 32, 34, 36, 38, 42) demonstrated a saturated transfection rate (at least 90% positive cells) at 5 ng formulated RNA construct. Others (constructs 6, 29 and 41) exhibited a low transfection rate (below 25% positive cells) and others (RNA constructs 2, 8, 23, 24, 30, 31, 32, 33, 35, 37, 39, 45) were in an acceptable (i.e. intermediate) range (at least 40% positive cells). As shown in FIG. 2A, all polyribonucleotides encoding Plasmodium polypeptide constructs with a TM domain or GPI anchor (RNA constructs 22, 23, 25, 28, and 30-41) demonstrated positive surface expression. As shown in FIG. 2B, these polyribonucleotides encoding TM domain or GPI anchor-containing Plasmodium polypeptide constructs exhibited varying degrees of total expression, with intracellular staining detected at a median fluorescence intensity of 20,000 to 80,000 and surface staining detected with a median fluorescence intensity of 0 to about 20,000). When protein secretion was assessed by measuring protein levels in culture supernatants, of the polyribonucleotides encoding Plasmodium polypeptide constructs that contain a signal sequence, only constructs 24 and 29 were detected in the culture supernatant (FIG. 2C).

Interestingly, it was observed that certain RNA constructs with a viral secretion signal (e.g., a HSV secretion signal) had increased total expression relative to an otherwise identical RNA construct with a Pf secretion signal. For example, construct 45 (which includes a HSV secretion signal) had higher total intracellular expression compared to construct 2 (an otherwise identical construct with a Pf secretion signal) (FIG. 2B) despite these constructs having similar transfection rate (FIG. 2A).

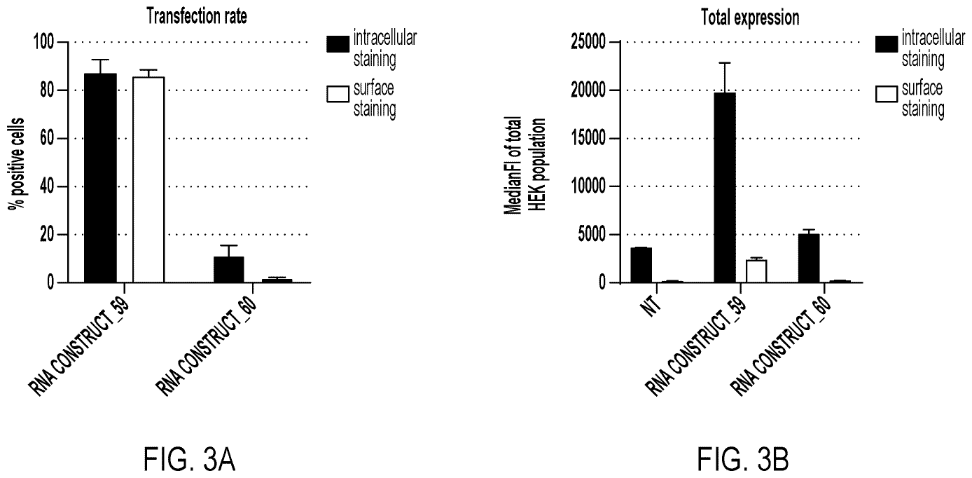

It was also observed that polypeptide expression from RNA construct 59 was at a higher level than RNA construct 60. RNA construct 59 also resulted in polypeptide expression that was detected on the surface of non-permeabilized cells (FIGS. 3A and 3B).

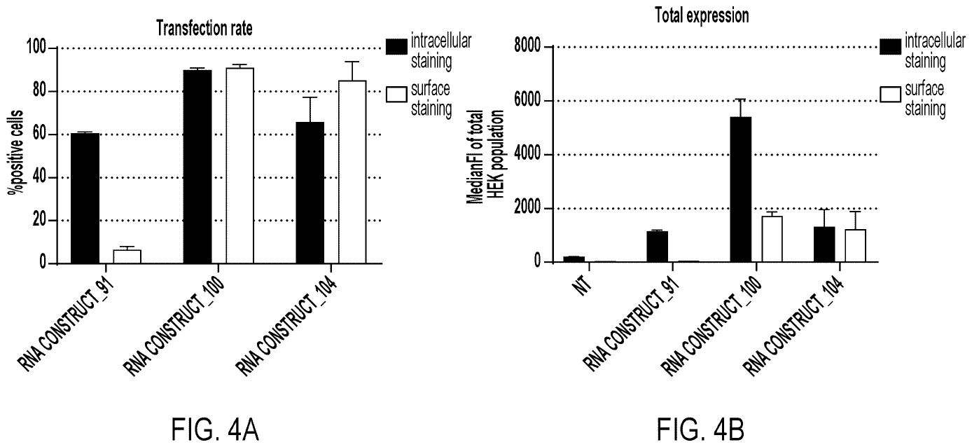

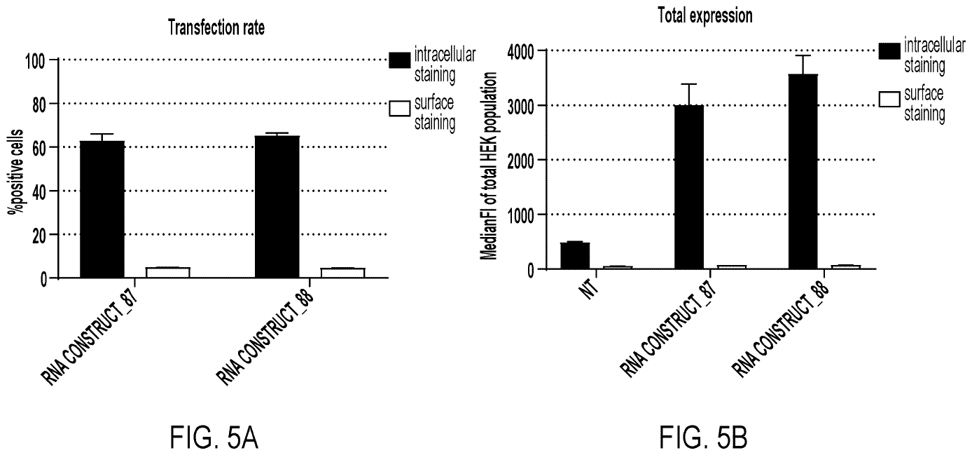

Compared between each other, out of RNA constructs 91, 100 and 104, RNA construct 100 demonstrated the highest in vitro expression. Both RNA construct 100 and RNA construct 104 had strong, and equal, surface expression (FIGS. 4A and 4B). Similar expression was seen for both RNA construct 87 and RNA construct 88 when compared to each other (FIGS. 5A and 5B); however, both were not expressed on the surface. LNP-formulated RNA constructs 87, 88, 91, 100 and 104 were also tested, at a concentration of 5 ng/well. RNA construct 100 and RNA construct 104 showed the highest level of expression when compared with the rest of the group (FIGS. 6A and 6B).

### Example 2: Immunogenicity Studies of Exemplary Polyribonucleotides Encoding Plasmodium Polypeptide Constructs

The present Example documents the ability of certain polyribonucleotides encoding Plasmodium polypeptide constructs, provided by the present disclosure, to induce immune responses, as assessed in mice.

C57BL6 female mice (10-12-weeks old) were immunized intramuscularly (IM) twice, on days 0 and 21, with 1 μg of a formulated RNA construct (described in Example 1) or injected with phosphate buffer saline (vehicle) (n=8 mice/group). Blood samples were collected pre-immunization (day 0) and after the first dose (on days 7, 14, 21, 28 and 35) to generate serum samples at various time points. At the end of the experiment (day 35), splenocytes were harvested and cryopreserved. Animals were divided into multiple groups receiving treatment as indicated in Table 10 below.

TABLE 10
Study plan for certain exemplary immunogenicity studies with RNA constructs

| Size | | | | | | | | |
|---|---|---|---|---|---|---|---|---|
| (n) | Group | Treatment | Dose | D 0 | D 7, 14, 21 | D 21 | D 28 | D 35 |
| 8 | 1 | Buffer | — | Blood collection | BC | IM | BC | Final |
| | | (NaCl) | | (BC)* | | | | Blood/Spleen |
| 8 | 2 | F1 | 1 μg | Intramuscular | | | | Collection |
| 8 | 3 | F2 | 1 μg | (IM) injection | | | | (FBC) |
| 8 | 4 | F3 | 1 μg | (20 μL in one leg) | | | | |
| 8 | 5 | F4 | 1 μg | | | | | |

F1, F2, F3, F4 = LNP formulations comprising RNA construct with modified nucleotides
BC = Blood collection/Serum generation (amount of blood that can be collected: 14 days interval between collections: 150 μl/20 g mouse; 7 days interval between collections: 110 μl/20 g mouse)
*pre-immune serum: blood sample is collected from 10% of all animals = 4 animals.
FBC = Final blood collection/Serum generation; Spleen collection and freezing of splenocytes (for Fluorospot analysis)

Serum samples obtained from each group of immunized animals were analyzed by one or more of the following method(s): (1) Enzyme-linked Immunosorbent Assay (ELISA), (2) multiplex assay, (3) sporozoite ELISA, (4) traversal Assay, (5) inhibition of Liver Stage Development Assay (ILSDA), and/or (6) Fluorospot assay.

### (1) Enzyme-Linked Immunosorbent Assay (ELISA)

Provided polyribonucleotides can be assessed for their ability to induce production of antibodies that may bind to Plasmodium falciparum (Pf) CSP full length protein (“PfCSP-FL”), PfCSP C-terminal domain (“PfCSP-C”), and/or the region spanning the end of the N-terminal domain until the end of the minor repeats (“PfCsp-76 to140”). In some embodiments, a provided polyribonucleotide is determined to induce a useful immune response if serum from a subject (e.g., a mouse) immunized with such construct is shown to bind PfCSP-FL, PfCSP-C, and/or PfCsp-76 to140 in an ELISA assay as described herein.

RNA constructs 2, 6, 8, 22-26, 28-31, 33-35, 37, 39, 41, 42, and 45 (as described in Example 1) were assessed for their ability to induce production of antibodies that bind to PfCSP-FL, PfCSP-C, and/or PfCsp-76 to140 using an ELISA assay (see Table 11).

RNA constructs 87, 88, 91, 100 and 104 were assessed for their ability to induce production of antibodies that bind to PfCSP-FL and the PfCSP C-terminal domain from 3D7 strain (PfCSP-C term 3D7) (see Table 11).

TABLE 11
List of P/CSP recombinant proteins and peptides assessed by ELISA analyses in the present Example

| Protein | Target | AS | | | | SEQ ID |
|---|---|---|---|---|---|---|
| target | region | sequence | Host | Tag | Sequence | NO.: |
| PICSP | Full length | 21-376 | Expi293 | His-tag (C- | MGGAAARLGAVILFVVIVGLHGVRGQEYQCY | 382 |
| | | | cells | terminal) | GSSSNTRVLNELNYDNAGTNLYNELEMNYY | |
| | | | | | GKQENWYSLKKNSRSLGENDDGNNEDNEKL | |
| | | | | | RKPKHKKLKQPADGNPDPNANPNVDPNANP | |
| | | | | | NVDPNANPNVDPNANPNANPNANPNANPNA | |
| | | | | | NPNANPNANPNANPNANPNANPNANPNANP | |
| | | | | | NANPNANPNANPNANPNANPNVDPNANPNA | |
| | | | | | NPNANPNANPNANPNANPNANPNANPNANP | |
| | | | | | NANPNANPNANPNANPNANPNANPNANPNA | |
| | | | | | NPNANPNKNNQGNGQGHNMPNDPNRNVD | |
| | | | | | ENANANSAVKNNNNEEPSDKHIKEYLNKIQN | |
| | | | | | SLSTEWSPCSVTCGNGIQVRIKPGSANKPKD | |
| | | | | | ELDYANDIEKKICKMEKCSS | |
| PICSP | C-terminus | 336-412 | Yeast | 6xHis | LKKIKNSISTEWSPCSVTCGNGIQVRIKPGSA | 383 |
| | | | | | NKPKDELDYENDIEKKICKMEKCSSVFNVVN | |
| | | | | | SSIGLIMVLSFLFLN | |
| PICSP | N-term | 76-140 | — | — | DDGNNEDNEKLRKPKHKKLKQPADGNPDPN | 384 |
| | end until | | | | ANPNVDPNANPNVDPNANPNVDPNANPNAN | |
| | minor | | | | PNANPA | |
| | repeats | | | | | |
| | (overlapping | | | | | |
| | peptides | | | | | |
| | 15/11 mer) | | | | | |
| PICSP | C-terminus | 273-376 | E. coli | His-tag (C- | NKNNQGNGQGHNMPNDPNRNVDENANAN | 439 |
| | (3D7) | | | terminal) | SAVKNNNNEEPSDKHIKEYLNKIQNSLSTEW | |
| | | | | | SPCSVTCGNGIQVRIKPGSANKPKDELDYAN | |
| | | | | | DIEKKICKMEKCSS | |

Briefly, MaxiSorp 96-well plates were coated with 100 ng/well of PfCSP-FL, PfCSP-C, or PfCSP-C term 3D7 in coating buffer (50 mM sodium carbonate, pH 9.6) and incubated overnight at 4° C., or 250 ng/well of PfCsp-76 to140 overlapping peptides in PBS and incubated 1 h at 37° C. Plates were then blocked with 1% BSA in PBS for 1 h at 37° C. (for PfCSP-FL, PfCSP-C, and PfCSP-C term 3D7) or overnight at 4° C. (for PfCsp-76 to140 overlapping peptides). Bound IgG was detected using horseradish peroxidase (HRP)-conjugated goat anti-mouse IgG. Signal was detected after adding the substrate 3,3′,5,5′-Tetramethylbenzidine (TMB) and 25% sulfuric acid to stop the reaction. Optical densities (OD) were read at 450 nm.

Reciprocal end titers at day 35, after 2 immunizations, were used as a representative as they showed the highest antibody response.

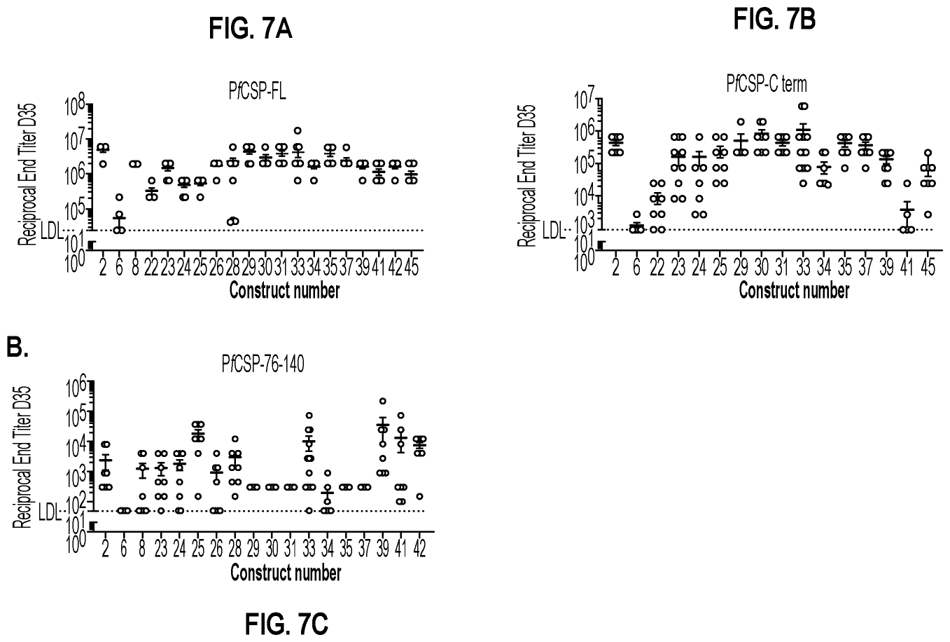

As shown in FIG. 7A, RNA constructs 2, 8, 22-26, 28-31, 33-35, 37, 39, 41, 42, and 45 induced high levels of antibodies to PfCSP-FL (exhibiting a mean reciprocal end titer of about 105 to about 107). As shown in FIG. 7B, RNA constructs 2, 8, 22-26, 28-31, 33-35, 37, 39, 41, 42, and 45 induced a strong antibody response to PfCSP-C(exhibiting a mean reciprocal end titer of about 105 to about 107). As shown in FIG. 7C, tested RNA constructs were more variable in their ability to induce antibodies to PfCsp-76 to140; furthermore, greater variability in antibody response to PfCSP-76 to140 was observed even among mice immunized with the same RNA construct, than was seen for responses to PfCSP-FL. Still, most tested RNA constructs (e.g., RNA constructs 2, 8, 22-26, 28, 29, 33-35, 37, 39, 41, 42, and 45) induced a significant antibody response even to PfCSP-76 to140 (exhibiting a mean reciprocal end titer of at least about 103) (data not shown). Use of PfCSP-76 to140 was discontinued due to high variability.

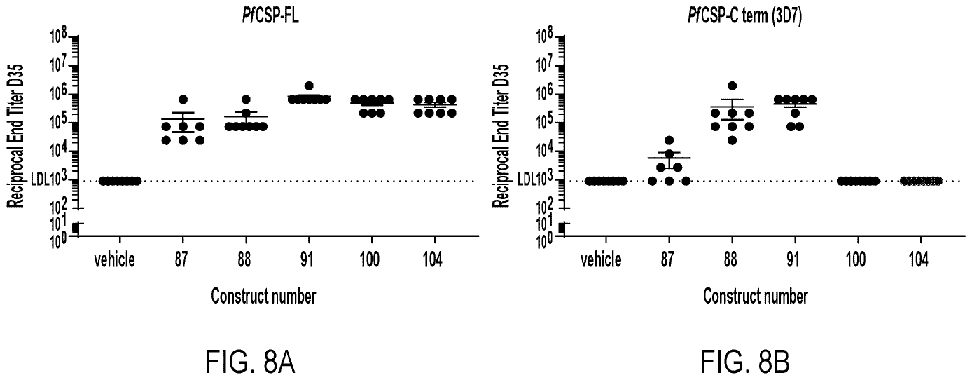

As shown in FIG. 8A, RNA constructs 87, 88, 91, 100 and 104 induced high levels of antibodies to PfCSP-FL (exhibiting a mean reciprocal end titer of about 105 to about 106). As shown in FIG. 8B, RNA constructs 87, 88, and 91 induced a strong antibody response to PfCSP-C term 3D7 (exhibiting a mean reciprocal end titer of about 104 to about 106). Immunization with RNA constructs 100 and 104 did not elicit antibodies against PfCSP-C term 3D7 (FIG. 8B) as expected, as neither of these constructs expresses this region of the protein. Immunization with RNA construct 87 elicited lower titers against the C-terminal domain of PfCSP than RNA construct 88 and RNA construct 91. No titers were detected for control mice (injected with the vehicle).

Thus, the present Example demonstrates that certain provided polyribonucleotides effectively induce an immune response characterized by inducing production of antibodies that bind to PfCSP-FL, PfCSP-C, PfCsp-76 to140, and/or PfCSP-C term 3D7, as assessed using an ELISA assay. All but one construct induced antibodies that bind to at least one of PfCSP-FL, PfCSP-C, PfCsp-76 to140, or PfCSP-C term 3D7. For example, polyribonucleotides that encoded Plasmodium polypeptide constructs that included a signal peptide (RNA constructs 2, 8, 23, 24, 25, 26, 28, 29, 30, 31, 33, 34, 35, 37, 39, 41, 42, 87, 88, 81, 100 and 104) are shown to have induced a strong antibody response to PfCSP-FL (mean reciprocal end titer of about 105 to about 107); polyribonucleotides encoding Plasmodium polypeptide constructs that included a signal peptide (RNA constructs 2, 29, 30, 31, 33, 35, 37, 88 and 91) are shown to have induced a strong antibody response to PfCSP-C(mean reciprocal end titer of about 105 to about 107); and polyribonucleotides encoding Plasmodium polypeptide constructs that included a proteolytic cleavage site (KLKQP (SEQ ID NO: 413); constructs 2, 8, 23, 24, 25, 26, 28, 33, 34, 39, 41, and 42) are shown to have induced a strong antibody response to PfCsp-76 to140 (mean reciprocal end titer of at least about 103).

### (2) Multiplex Assay

Provided polyribonucleotides can be assessed for their ability to induce production of antibodies that bind to specific PfCSP epitopes. In some embodiments, a provided polyribonucleotide is determined to induce a useful immune response if serum from a subject (e.g., a mouse) immunized with such construct is shown to target peptides from the central region of PfCSP (e.g., PfCSP peptide 17C, 18C, 19C, 20C, 21C, 22C, 23C, 27C, 29C, and/or 42C) in a multiplex assay, as described herein.

RNA constructs (as described in Example 1) were assessed for their ability to induce production of antibodies that bind to specific PfCSP epitopes (see Table 12) by performing a multiplex analysis (Meso Scale Discovery) according to the manufacturer's instructions. Briefly, ten peptides from the central region of PfCSP (PfCSP peptide 17C, 18C, 19C, 20C, 21C, 22C, 23C, 27C, 29C, or 42C) were conjugated with bovine serum albumin (BSA) and then bound to the wells of a 96-well plate, in a specific spot on the well. After incubation with serum from immunized mice, antibodies bound to each specific peptide were detected with a “Sulfo-Tag” conjugated secondary antibody. A multiplex reader instrument (MESO QuickPlex SQ 120) was used to quantify the light emitted from the Sulfo-Tag.

TABLE 12
P/CSP peptides used in the multiplex analysis

| | AS | | SEQ ID |
|---|---|---|---|
| Designation | sequence | Sequence | NO.: |
| 17C | 87-101 | CRKPKHKKLKQPADGN | 385 |
| 18C | 91-105 | CHKKLKQPADGNPDPN | 386 |
| 19C | 95-113 | CKQPADGNPDPNANPNVDPN | 387 |
| 20C | 97-111 | CPADGNPDPNANPNVD | 388 |
| 21C | 101-115 | CNPDPNANPNVDPNAN | 389 |
| 22C | 105-123 | CNANPNVDPNANPNVD | 390 |
| 23C | 109-123 | CNVDPNANPNVDPNAN | 391 |
| 27C | 125-139 | CNVDPNANPNANPNAN | 392 |
| 29C | 129-143; | CNANPNANPNANPNAN | 393 |
| | 145-159; | | |
| | 161-175; | | |
| | 177-191; | | |
| | 201-215; | | |
| | 217-231; | | |
| | 233-247; | | |
| | 249-263 | | |
| 42C | 185-199 | CNVDPNANPNVDPNAN | 394 |

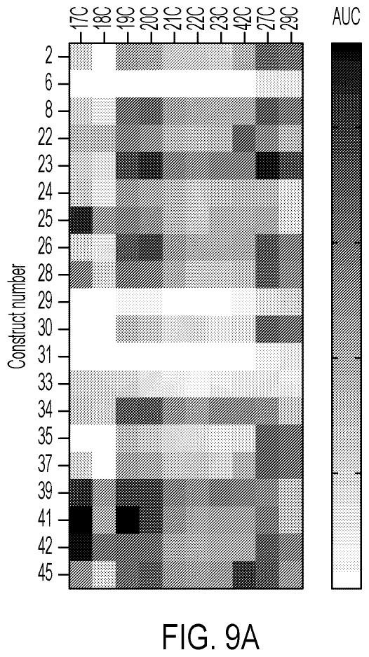

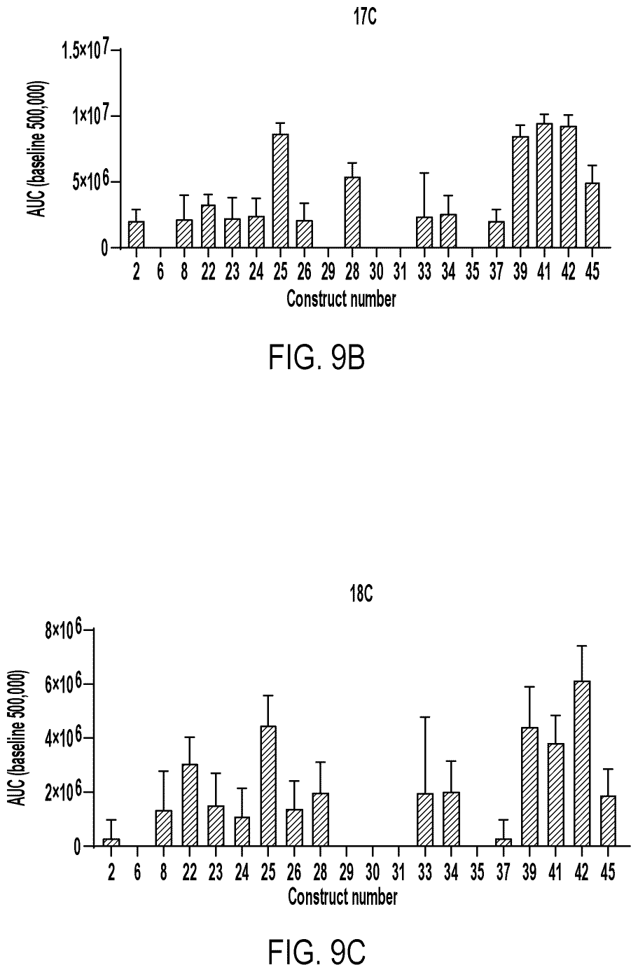

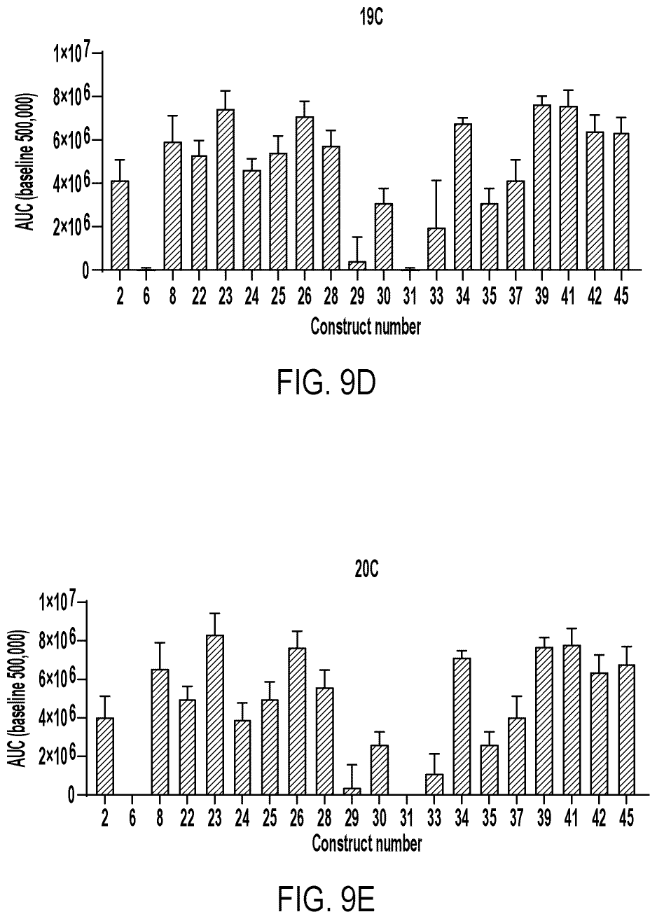

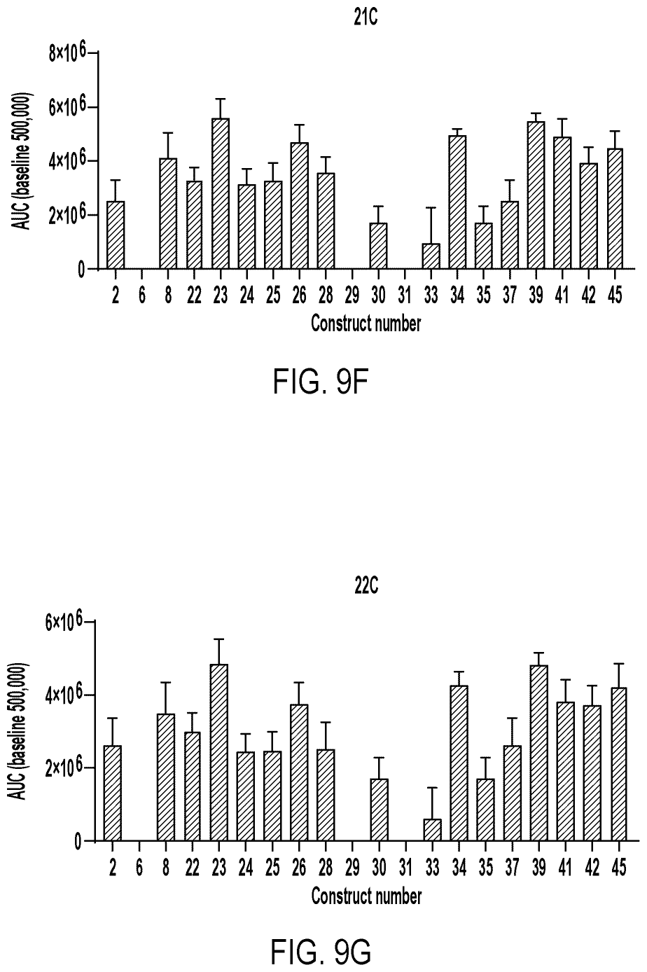

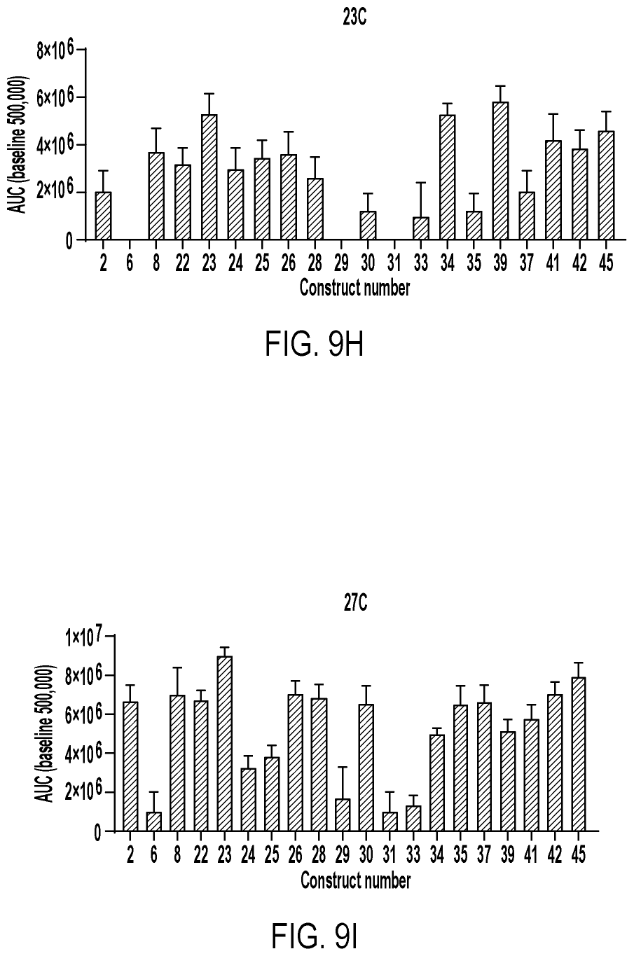

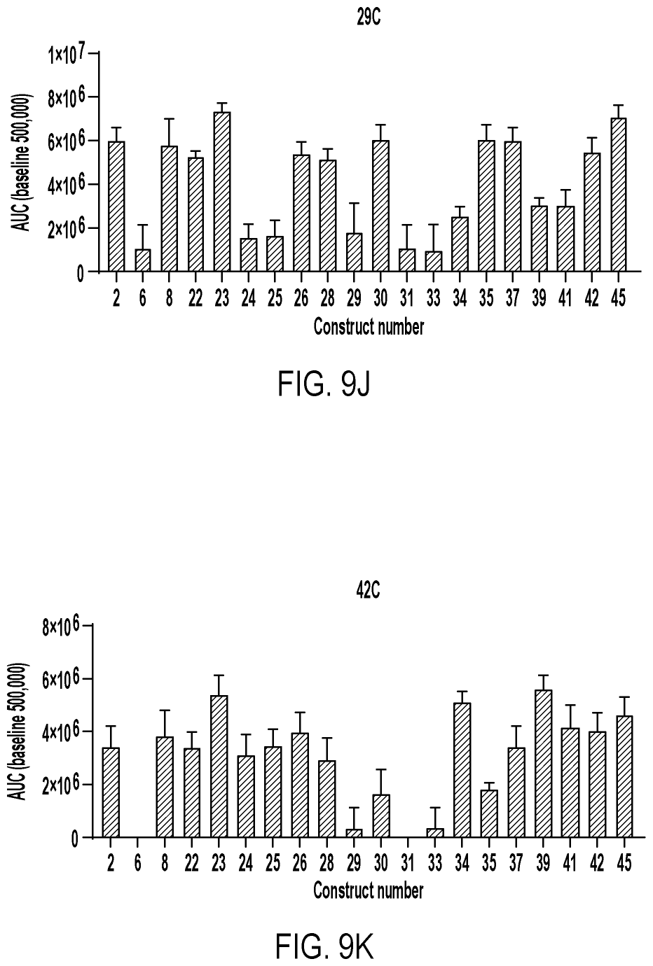

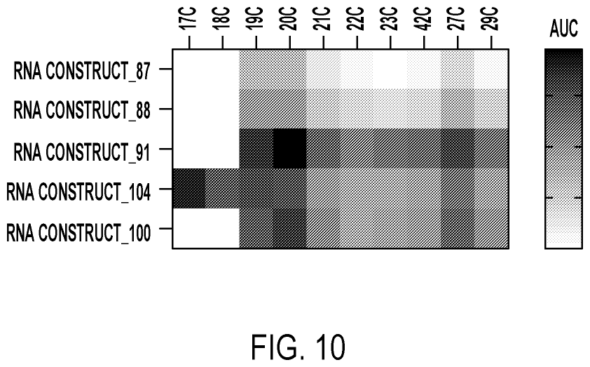

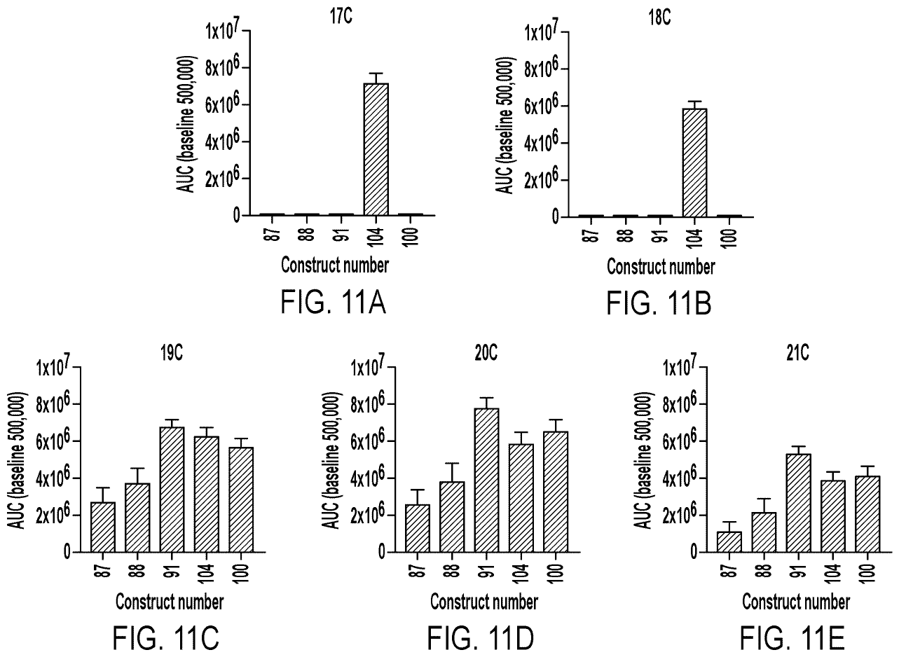

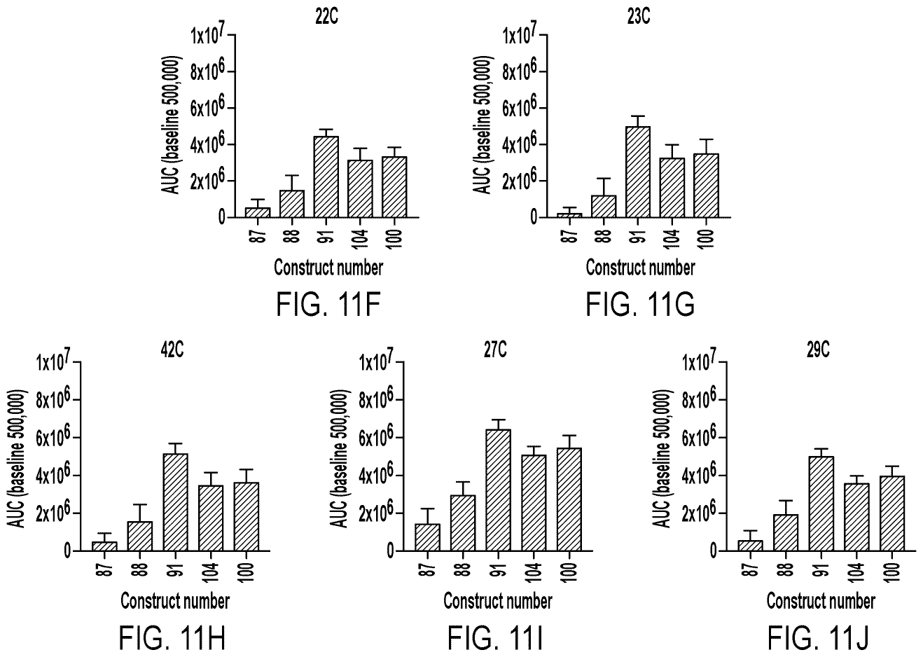

Most tested RNA constructs generated antibodies that exhibited binding to at least some epitopes (FIGS. 9-11).

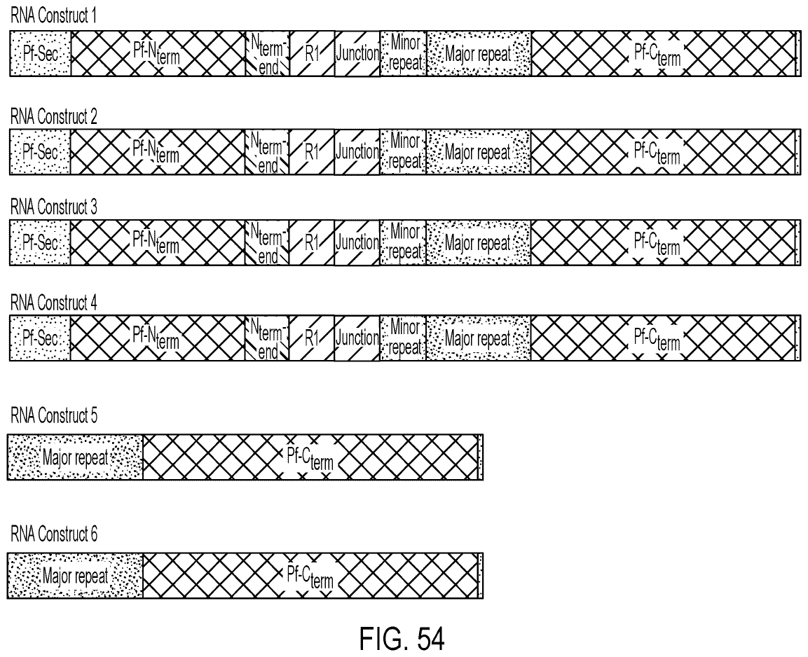

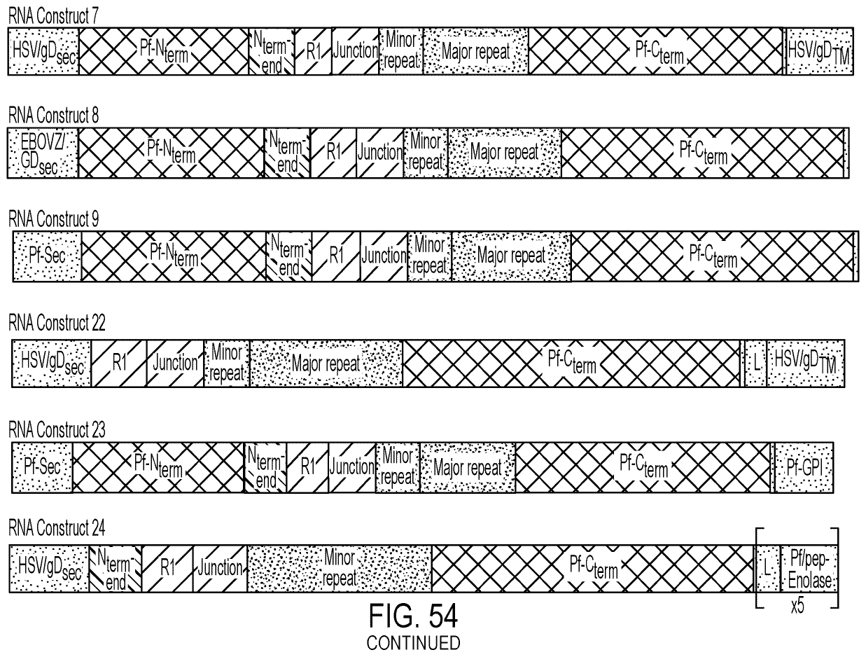

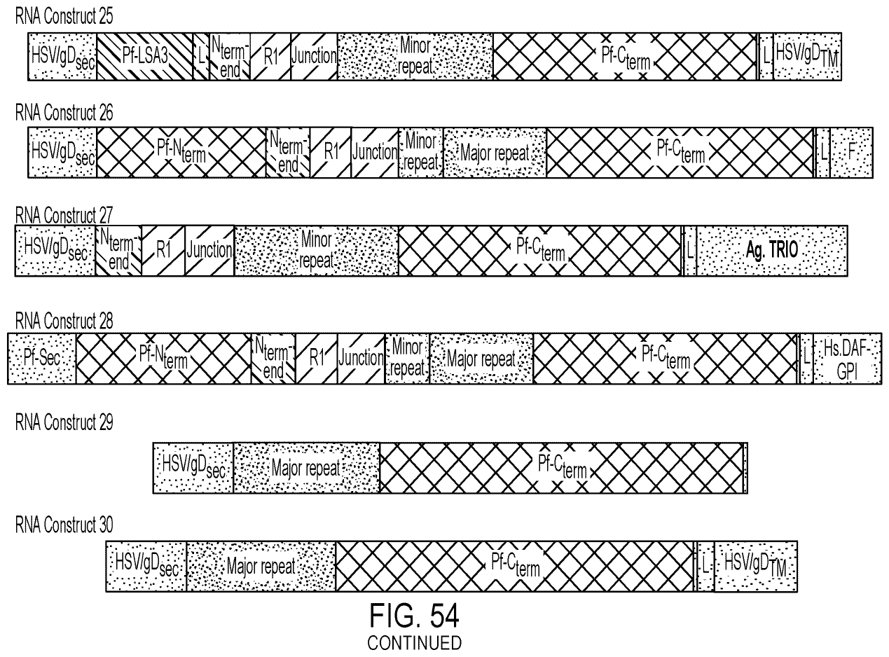

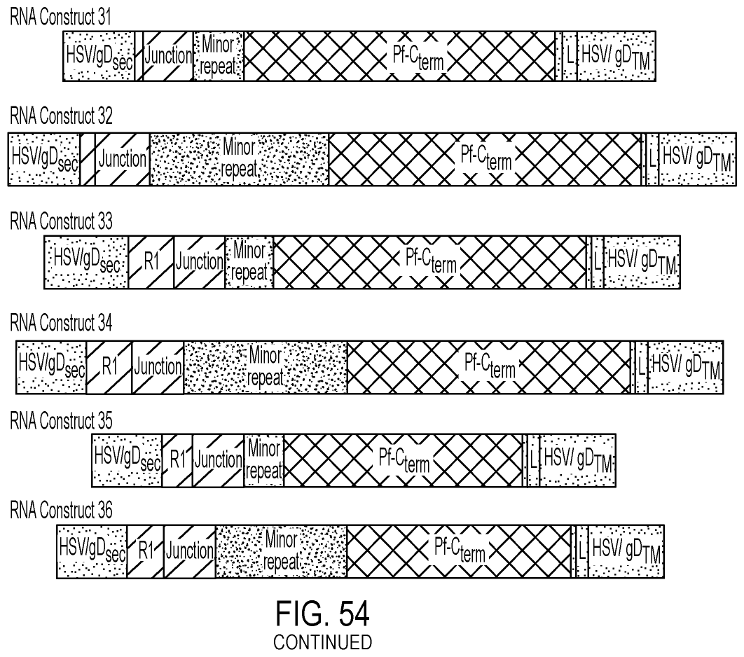

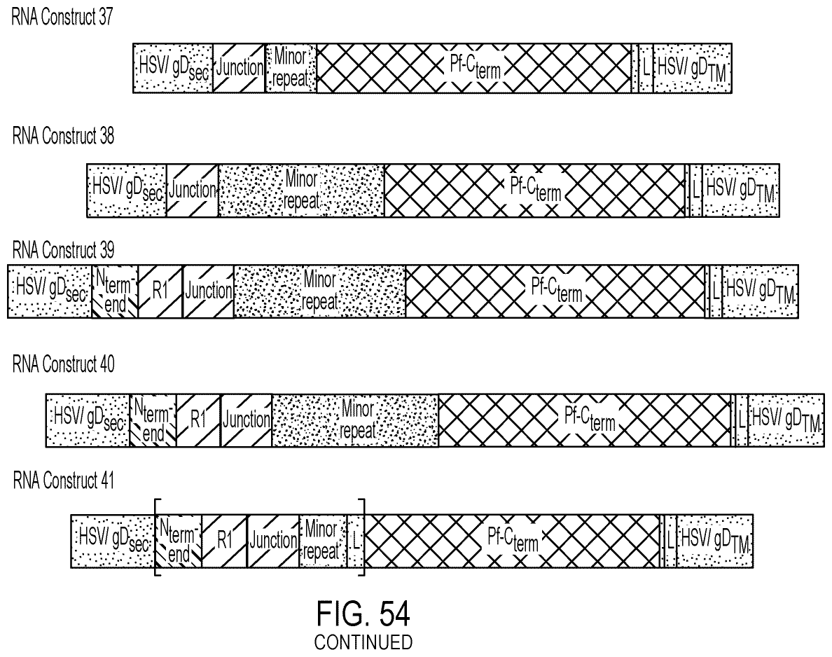

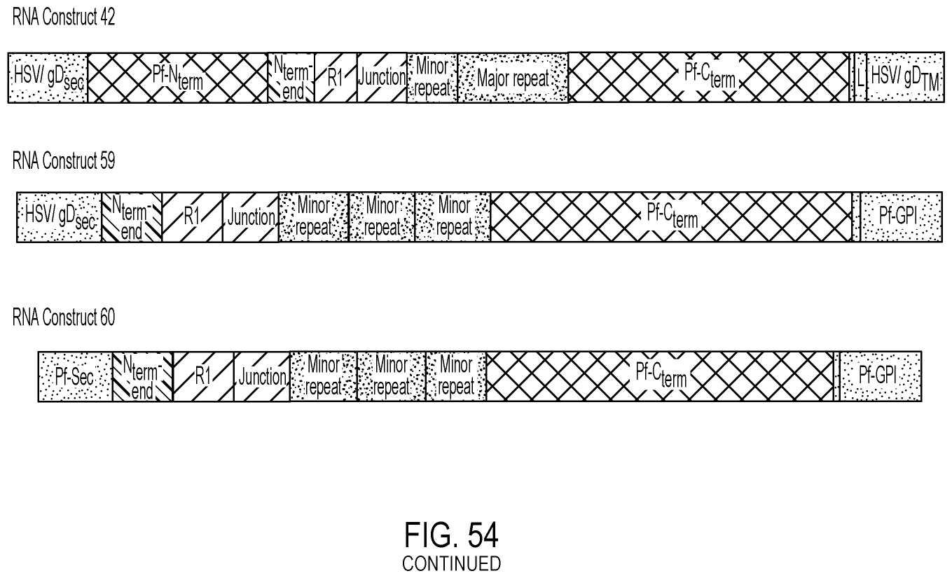

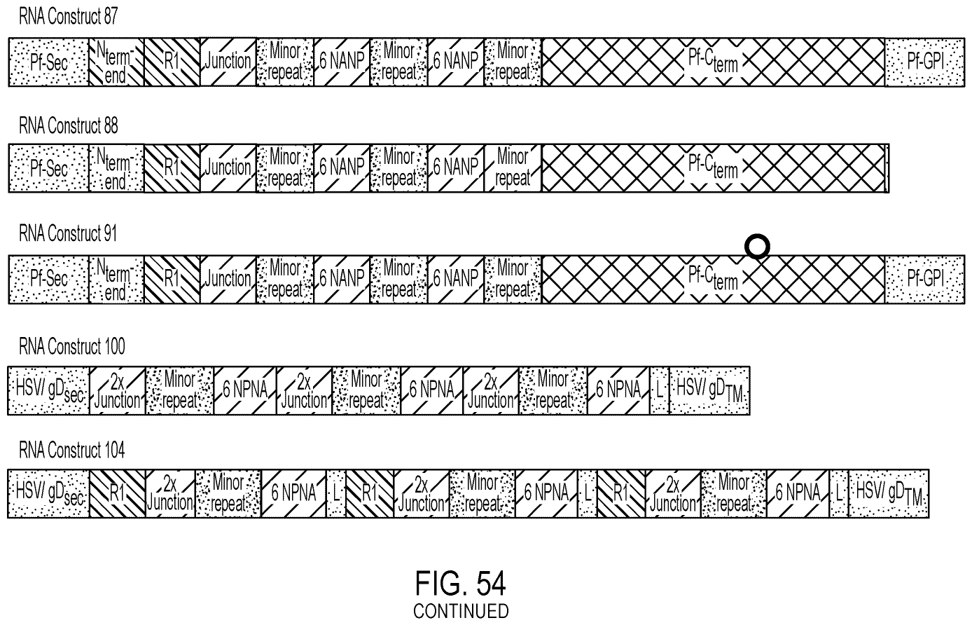

Peptides 17C and 18C are partially located in the N-terminal domain, R¹ and junction of the PfCSP and antibodies against these peptides and were recognized (demonstrating AUC of at least about 600,000) mainly by polyribonucleotides encoding full length Plasmodium polypeptide constructs (RNA constructs 2, 8, 23, 26, 28, 42, 45) and RNA constructs 22 (encoding a Plasmodium polypeptide construct AN-term), 25 (encoding a Plasmodium polypeptide construct which lacks major repeats and PILSA-3 fragment is in place of N-term), 41 (encoding a Plasmodium polypeptide construct in which the N-term, R¹ and junction region is repeated 4 times and the major repeats are left out), and 104 (encoding a Plasmodium polypeptide construct in which the R1, junction region, minor repeat, and 6 major repeats are repeated 3 times) (FIGS. 9-11; see also FIG. 54).

Peptides 19C and 20C span R1, junctional region and a part of minor repeats and were recognized by antibodies generated from polyribonucleotides encoding full length CSP constructs (RNA constructs 2, 8, 23, 26, 28, 42, and 45) (FIGS. 9-11). As shown in FIGS. 9 and 10, only RNA constructs 6, 29, and 31 did not induce antibodies against at least one of 17C, 18C, 19C or 20C.

All RNA constructs tested encode at least some part of the minor or major repeats of PfCSP, and antibodies to peptides 23C, 42C and 27C, which all span the minor and major repeats in different sections, were observed for most of the constructs (demonstrating AUC of at least about 600,000) (FIGS. 9-11).

Peptides 21C, 22C and 29C span the minor or major repeats and are the main binding epitopes of known neutralizing antibodies, CIS43, L⁹ and mAb317, respectively. Antibodies against these regions were produced by immunization with all RNA constructs, except RNA constructs 6, 29 and 31, which induced antibodies only to 29C (major repeats) (FIGS. 9-11).

Thus, the present Example demonstrates that all provided polyribonucleotides effectively induce production of antibodies that bind at least to one specific PfCSP epitopes from the central region of PfCSP (e.g., PfCSP peptide 17C, 18C, 19C, 20C, 21C, 22C, 23C, 27C, 29C, and/or 42C).

### (3) Sporozolte ELISA

Provided polyribonucleotides can be assessed for their ability to induce production of antibodies that may bind to native CSP antigen from Plasmodium falciparum (Pf) sporozoite lysates. In some embodiments, a provided polyribonucleotide is determined to induce a useful immune response if serum from a subject (e.g., a mouse) immunized with such construct is shown to bind to native CSP antigen from Plasmodium falciparum (Pf) sporozoite lysates in a sporozoite ELISA assay, as described herein.

RNA constructs (as described in Example 1), were assessed for their ability to induce production of antibodies that bind to native CSP antigen from Plasmodium falciparum (Pf) sporozoite lysates, using a sporozoite ELISA assay. Specifically, 384-well plates were coated with non-denatured total protein lysate from Plasmodium falciparum (Pf) NF54 salivary gland sporozoites, in an amount equivalent to about 250 sporozoites per well. Following blocking, serially diluted serum samples (6 dilutions per sample) were added to the corresponding wells. Binding of antibodies present in the serum samples to the native PfCSP protein in the wells was detected using an AP-conjugated secondary antibody followed by luminescent quantification.

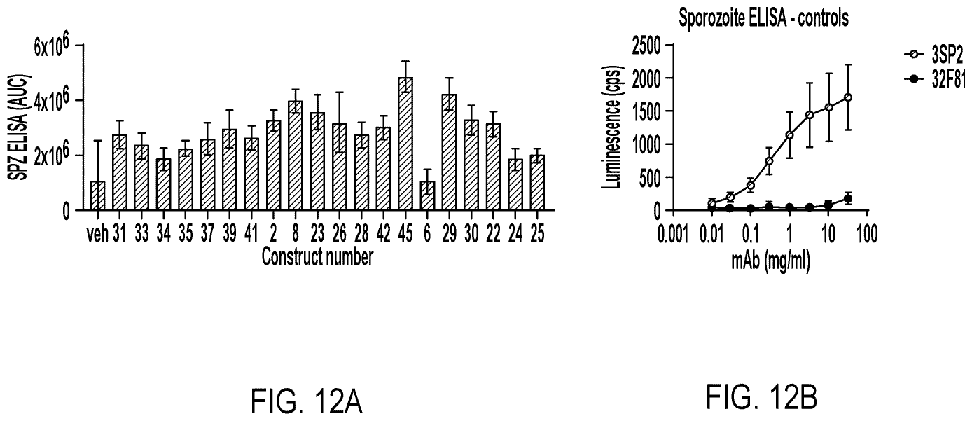

As shown in FIG. 12A, tested RNA constructs effectively induced production of antibodies that bound native PfCSP antigen (exhibiting luminescence of at least about 200 cps). Overall, RNA constructs encoding Plasmodium polypeptide constructs including full length PfCSP (RNA constructs 2, 8, 23, 26, 28, 42, and 45) performed better than others (exhibiting luminescence of about 300 cps to about 700 cps). Interestingly, it was observed that certain RNA constructs with a viral secretion signal (e.g., a HSV secretion signal) had improved production of antibodies that bound native PfCSP antigen relative to an otherwise identical RNA construct with a Pf secretion signal. For example, FIG. 12A shows that construct 45 (which includes a HSV secretion signal) had higher luminescence than construct 2 (an otherwise identical construct with a Pf secretion signal). FIG. 12B shows binding of murine anti-Pfs25 mAb32F81 used as negative control, and murine anti-CSP mAb3SP2 used as a positive control.

Thus, the present Example demonstrates that certain provided polyribonucleotides effectively induce an immune response characterized by inducing production of antibodies that bind to native CSP antigen from Plasmodium falciparum (Pf) sporozoite lysates in a sporozoite ELISA assay. All but one tested constructs showed higher antibodies compared to the vehicle. For example, RNA constructs 2, 8, 22, 23, 26, 28, 29, 33, 34, 35, 39, 41, 42 and 45 effectively induced production of antibodies that bind native PfCSP antigen (exhibiting luminescence of at least about 200 cps), and in particular, RNA constructs encoding full length PfCSP (constructs 2, 8, 23, 26, 28, 42, and 45) exhibited luminescence of about 300 cps to about 700 cps.

### (4) Traversal Assay

Provided polyribonucleotides can be assessed for their ability to induce production of antibodies with an inhibitory effect on traversal (a type of motility displayed by Plasmodium falciparum (Pf) sporozoites that is essential for their infectivity). In some embodiments, a provided polyribonucleotideis determined to induce a useful immune response if serum from a subject (e.g., a mouse) immunized with such construct is shown to reduce ability of sporozoite to traverse hepatocytes in a traversal assay, as described herein.

RNA constructs (as described in Example 1) were assessed for their ability to induce production of antibodies that have an inhibitory effect on Plasmodium falciparum (Pf) sporozoites traversal. Briefly, HC-04 cells, a human hepatocyte cell line, were seeded into plates and incubated for 24 h at 5% CO2 and 37° C. Freshly isolated Plasmodium falciparum (Pf) salivary gland sporozoites were pre-incubated with serially diluted (1:20, 1:80 and 1:320) serum samples from mice immunized with formulated RNA constructs. Sporozoites were then added to the HC-04 cells in a multiplicity of infection (MOI) of 1:1, in the presence of impermeable dye dextran-rhodamine. As a positive control for inhibition, sporozoites were pre-treated with mAb317. Non-treated sporozoites were used as a negative control for inhibition. Ability of Plasmodium falciparum (Pf) sporozoites to traverse cells was quantified by determining a percentage of cells that incorporated dextran-rhodamine, by fluorescence microscopy. Sporozoite traversal of sporozoites pre-incubated with serum samples from mice injected with vehicle was set as 0% traversal inhibition.

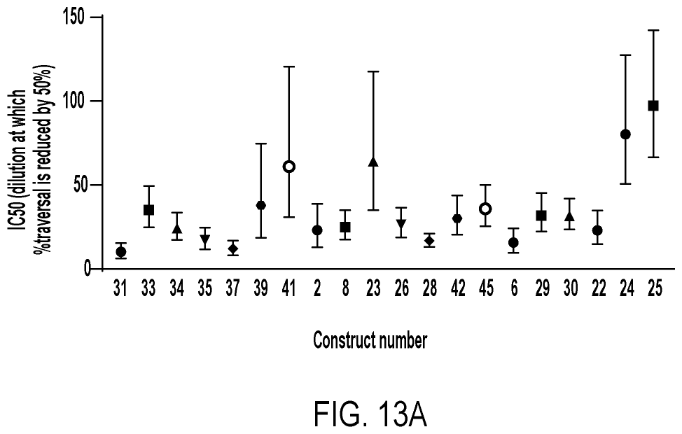

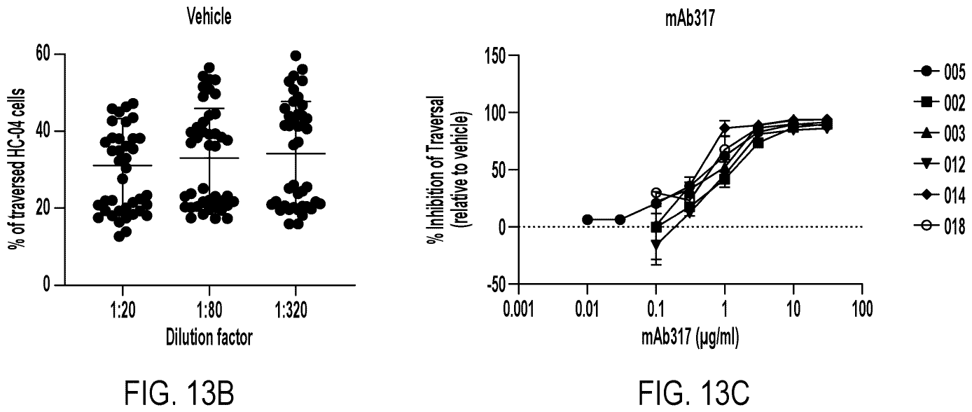

When sporozoites were pre-incubated with serum from mice injected with vehicle, about 50% of the cells incorporated dextran-rhodamine, indicating that they were traversed by sporozoites (see FIG. 13B). The average of all vehicle samples was considered as 0% inhibition and was a comparator for all samples from mice immunized with RNA constructs.

At lower (1:20) dilution of sera, there was about 60-80% inhibition of traversal observed for sera from mice immunized with RNA constructs 2, 23, 26, 33, 39, 41, and 42, while sera from mice immunized with other RNA constructs inhibited 30-50% traversal. As the dilution increased, there was a decrease in the percentage inhibition for most of the constructs. At a 1:80 dilution, sera from mice immunized with RNA construct 2, 33, or 42 was able to inhibit traversal by about 20-30%, and sera from mice immunized with RNA construct 23, 39, or 41 was able to inhibit traversal by about 50-60%. At a higher dilution (1:320), sera from mice immunized with RNA construct 23, 39, or 41 was able to inhibit traversal by 50-60%, while sera from mice immunized with other RNA constructs was able to inhibit traversal by under 40%.

As shown in FIG. 13A, the antibodies generated from mice immunized with different formulated RNA constructs are able to inhibit P. falciparum sporozoite traversal. Results are shown as the percentage of inhibition of traversal activity (mean with SEM) in comparison to the vehicle control, which was set as 0% inhibition. The RNA constructs 39, 41, 23, 24 and 25 are the ones with higher traversal inhibitory activity. FIG. 13B shows the percentage of traversed cells for the vehicle and inhibition of traversal by mAb317 relative to the vehicle.

Thus, the present example demonstrates that certain polyribonucleotides effectively induce an immune response characterized by inducing production of antibodies that inhibit sporozoite traversal, e.g., as measured using a traversal assay. For example, sera from mice immunized with RNA constructs 2, 23, 26, 33, 39, 41, and 42 inhibited traversal by about 60-80% at 1:20 serum dilution, while sera from mice immunized with RNA constructs 23, 39, and 41 inhibited traversal by about 50-60% at 1:320 dilution.

### (5) Inhibition of Liver Stage Development Assay (ILSDA)

Provided polyribonucleotides can be assessed for their ability to induce production of antibodies that inhibit infection of primary human hepatocytes by Plasmodium falciparum (Pf) sporozoites, and/or their development therein. In some embodiments, a provided polyribonucleotideis determined to induce a useful immune response if serum from a subject (e.g., a mouse) immunized with such construct is shown to inhibit Plasmodium falciparum (Pf) sporozoite infection and/or development in an ILSDA assay as described herein.

In the ILSDA assay, freshly isolated salivary gland sporozoites were pre-incubated with serially diluted serum samples (8 dilutions) of sera from mice immunized with RNA constructs (as described in Example 1). Pre-incubated sporozoites were then added to primary human hepatocytes that were seeded on a glass-bottom black 96-well plate. After centrifugation to facilitate infection, cells were incubated for 4 days at 5% CO2 and 37° C. After fixation, parasite cytoplasm was stained with anti-PfHsp70 and DNA (from hepatocytes and from parasites) was stained with DAPI. mAb317, an antibody known to inhibit hepatocyte infection, was used as positive control. Sporozoites incubated with serum from mice injected with vehicle were used as a negative control for inhibition. Ability of Plasmodium falciparum (Pf) sporozoites to infect hepatocytes was quantified by determining a percentage of cells with parasites inside, by fluorescence microscopy. Parasite development is also assessed by measuring the area of the parasites (stained with anti-PfHsp70).

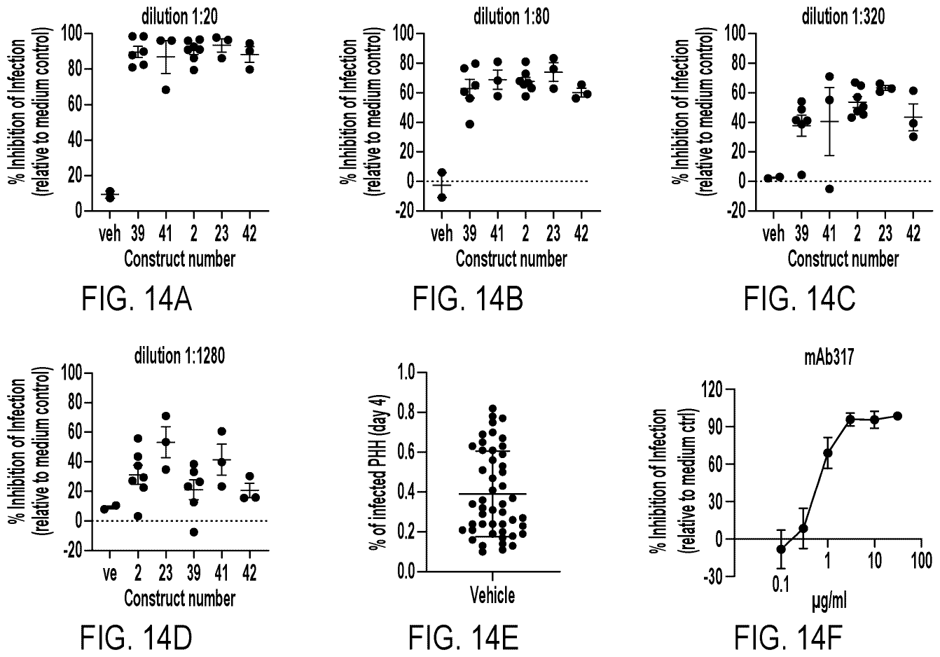

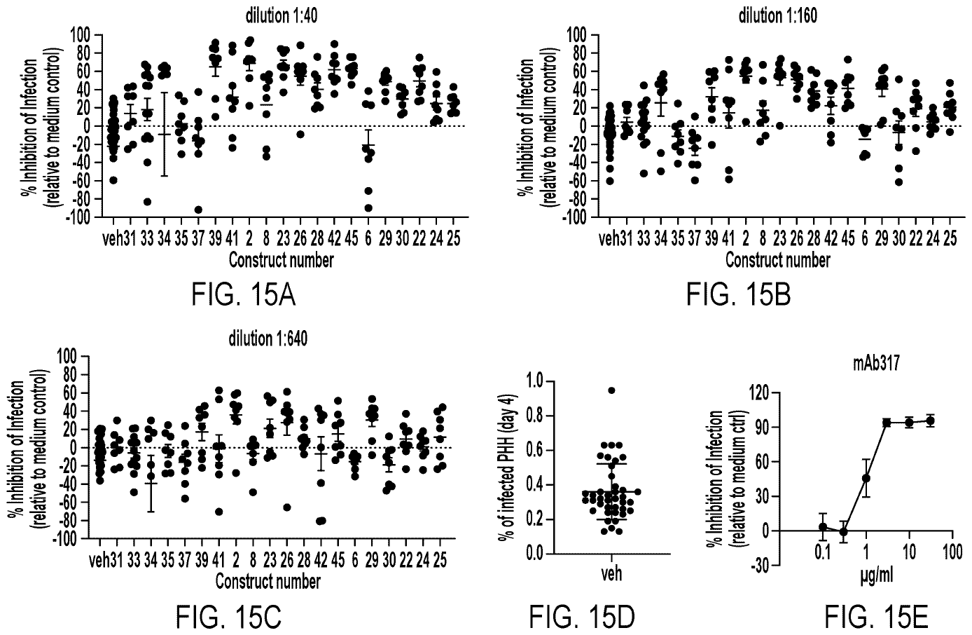

As shown in FIG. 14A, at lower (1:20) dilution of sera from mice immunized with all tested RNA constructs primary human hepatocyte infection was inhibited around 80-100%. The percentage inhibition decreased to between 60-80% for all tested samples with the increase in sera dilution to 1:80. At serum dilution of 1:320 differences were seen in percentage of hepatocyte inhibition, where sera from mice immunized with polyribonucleotides encoding full length CSP constructs (RNA constructs 2 and 23) were able to inhibit infection about 50-70%, while sera from mice immunized with RNA construct 39, 41 and 42 were able to inhibit around 40% of infection. At a higher dilution (1:1280), sera from mice immunized with RNA construct 23 was able to inhibit infection by 50-60%, while sera from mice immunized with other RNA constructs was able to inhibit infection by under 40%. As shown in FIG. 15A, at lower (1:40) dilution of sera from mice immunized with all tested RNA constructs 39, 2, 23, 26, 42, 45, 29 and 22 inhibited, primary human hepatocyte infection was inhibited above 50% and RNA constructs 31, 33, 34, 39, 41, 2, 8, 23, 26, 28, 42, 45, 29, 30, 22, 24 and 25 human hepatocyte infection could be observed. The percentage inhibition decreased for all tested samples with the increase in sera dilution to 1:160 with RNA constructs 2, 23, 26, 28, 45 and 29 showing inhibition around or above 40%. At dilution 1:640, inhibition is only detectable for constructs 39, 2, 23, 26, 28, 45, 29, 22 and 25.

Thus, the present example demonstrates that certain polyribonucleotides effectively induced production of antibodies that inhibited invasion of primary human hepatocytes, e.g., as measured using an inhibition of liver stage development assay. For example, sera from mice immunized with RNA constructs 2, 23, 39, 41, and 42 were able to inhibit infection at lower dilutions (1:20), while sera from mice immunized with RNA construct 23 inhibited infection over 40% at higher dilution (1:1280).

### (6) Complement-mediated Lysis of Sporozoites

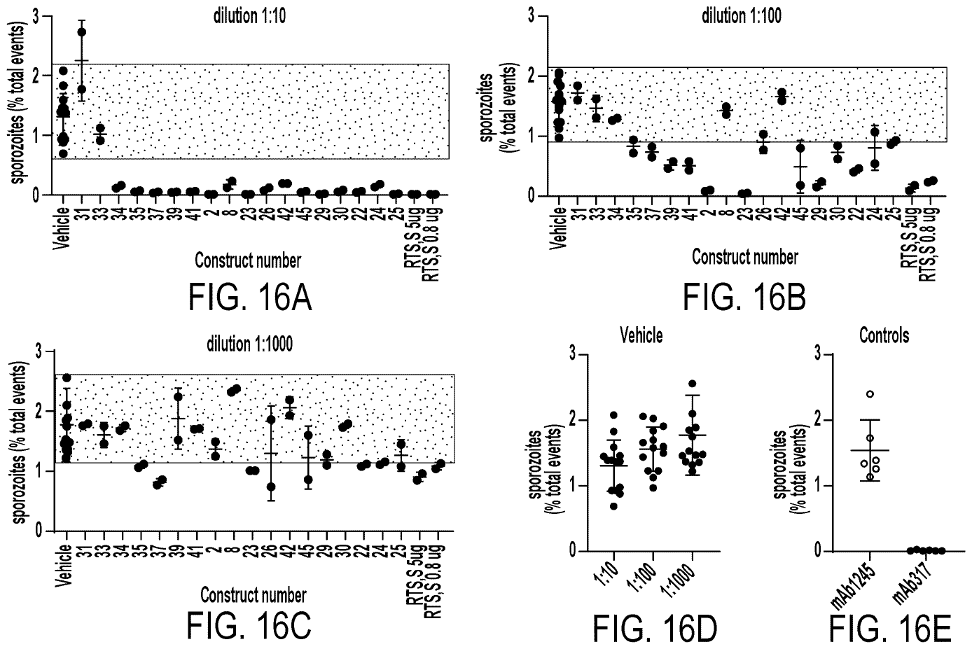

Sporozoites were incubated with serum from mice immunized with RNA constructs 2, 8, 22, 23, 24, 25, 26, 29, 30, 31, 33, 34, 35, 37, 39, 41, 42, and 45, and with normal human serum (NHS), as source of complement, at 37° C. for 30 minutes (FIG. 16A-16E). Monoclonal antibodies mAb1245 and mAb317 (10 g/ml) were used as negative and positive control, respectively (FIG. 16E). After inactivation of complement with PBS/EDTA, sporozoites were stained with 3SP2-DyLight650. Samples were then washed, fixed, washed again and resuspended in FACS buffer for flow cytometry analyses, which was performed by detecting the viable sporozoites (not lysed) with an anti-CSP antibody.

After an incubation with pooled mouse serum samples at a dilution 1:10, viability of sporozoites was highly impacted by most of the constructs, except RNA Constructs 31 and 33 (FIG. 16A). At a dilution 1:100, viability of sporozoites was less affected by most of the constructs, but RNA constructs 35, 37, 39, 41, 2, 23, 26, 45, 29, 30, 22, 24 and 25 reduced vability with RNA constructs 2, 23, 29 and 22 having a very high impact on sporozoite viability (FIG. 16B), similar to what was observed with Mosquirix®. At a dilution of 1:1000, the viability of sporozoites after incubation with pooled mouse serum is similar to that of the vehicles for most constructs, except for RNA constructs 35, 37, 23 and 22 (FIG. 16C), which were still able to impact sporozoite viability, similar to what was observed with Mosquirix®. A small unspecific effect on viability of sporozoites was observed for vehicle samples at low dilutions (FIG. 16D).

(7) Total Binding and Dissociation from PfCSP Full Length Protein or Specific Peptides

Binding and dissociation of the antibodies present in serum from mice immunized with RNA constructs 31, 33, 35, 37, 29, 30, 2, 42, 8, 26, 34, 39, 41, 23, 24, 25, 22, and 45 (as described in Example 1) to biotinylated full length PfCSP, junction+minor repeats peptide or major repeats peptide was determined by Surface Plasmon Resonance (SPR). Polypeptides used in the SPR measurements are provided in Table 13 below. Briefly, biotinylated full length PfCSP, junction+minor repeats peptide and major repeats peptide were each immobilized to a different flow cell of a Biacore T200 instrument, while the fourth flow cell was left empty as reference. Serum samples were diluted in analysis buffer (10 mM HEPES (pH 7.4), 150 mM NaCl, 3 mM EDTA, 0.05% Tween 20, 0.22 μm filtered) and ran using a flow rate of 10 μL/min for interaction analysis of the analyte (association and dissociation). Association of the antibodies in the serum was measured for 5 min, while dissociation was measured for 15 min. Analyzes were performed in triplicates with individually prepared dilutions. Buffer blanks were implemented regularly and used for referencing. Evaluation of serum sample binding data was performed with regard to two parameters: a) height of binding signals, as relative comparison of the titer, and b) assessment of antibody: antigen complex stability based on dissociation, by calculating the binding signal after 5 min and 15 min, which was determinized by calculating the dissociation relative to the maximal signal (% residual response). The higher the % residual response value the higher the complex stability.

TABLE 13
Polypeptides used in the SPR measurements

| | As | | |
|---|---|---|---|
| Designation | sequence | Tag | Sequence |
| PICSP-FL | 1-397 | His-tag | MMRKLAILSVSSFLFVEALFQEYQCYGSSSNTRVLNELNYDNAGTNLYNELEMNYY |
| | | (C- | GKQENWYSLKKNSRSLGENDDGNNEDNEKLRKPKHKKLKQPADGNPDPNANPN |
| | | terminal) | VDPNANPNVDPNANPNVDPNANPNANPNANPNANPNANPNANPNANPNANPNA |
| | | | NPNANPNANPNANPNANPNANPNANPNANPNANPNVDPNANPNANPNANPNAN |
| | | | PNANPNANPNANPNANPNANPNANPNANPNANPNANPNANPNANPNANPNANP |
| | | | NANPNKNNQGNGQGHNMPNDPNRNVDENANANSAVKNNNNEEPSDKHIKEYL |
| | | | NKIQNSLSTEWSPCSVTCGNGIQVRIKPGSANKPKDELDYANDIEKKICKMEKCSS |
| | | | VFNVVNSSIGLIMVLSFLFLN |
| Junction + | 81-128 | biotin | EDNEKLRKPKHKKLKQPADGNPDPNANPNVDPNANPNVDPNANPNVDP |
| minor | | | |
| repeats | | | |
| Major | multiple | biotin | NANPNANPNANPNANPNANPNANP |
| repeats | | | |

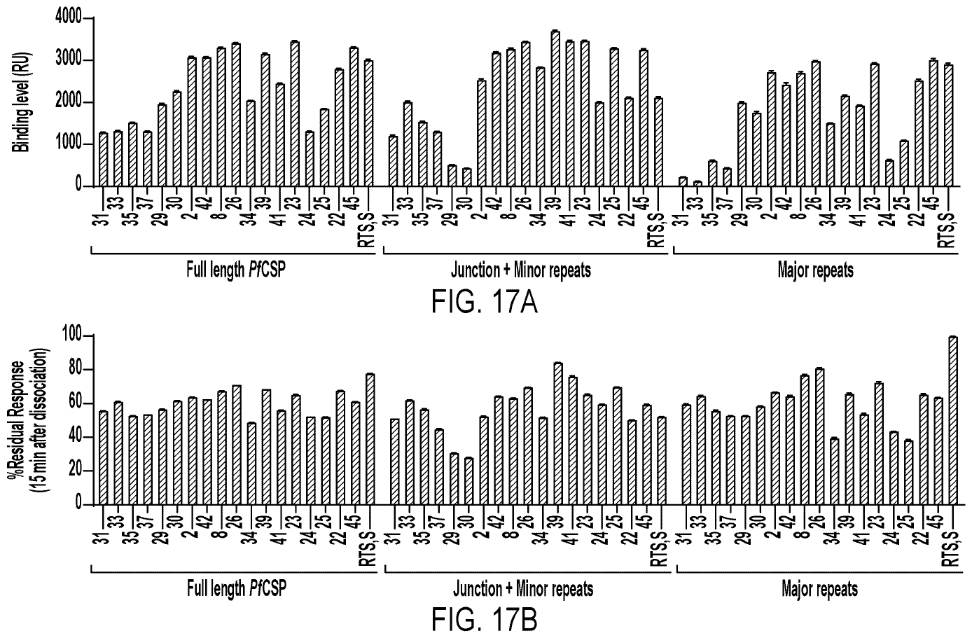

The ability of the antibodies generated upon immunization with the different constructs to bind full length PfCSP, junction+minor repeats peptide or major repeats peptide was assessed by SPR (FIG. 17A). The highest binding to full length PfCSP was observed for all full length constructs (RNA constructs 2, 42, 8, 26, 23 and 45), RNA construct 39, Mosquirix® and RNA construct 22. The highest binding to the junction+minor repeats peptide was observed for RNA construct 39, RNA construct 41, all full length constructs (RNA constructs 2, 42, 8, 26, 23 and 45), and RNA construct 25. Finally, the highest binding to the major repeats peptide was observed for all full length constructs (RNA constructs 2, 42, 8, 26, 23 and 45), Mosquirix® and RNA construct 22.

The dissociation of the antibodies generated upon immunization with the different constructs to the three antigens was also assessed by determining the percentage of residual binding after 15 min of dissociation, as a measurement of the strength of the interaction between antibodies and the antigens (FIG. 17B). All tested constructs showed 50% or more residual binding for full length PfCSP. The highest residual binding (which reflects slower dissociation) from full length PfCSP protein was observed for Mosquirix®, RNA constructs 26, 39, 8, 23, and 22. The highest residual binding to the junction+minor repeats peptide was observed for RNA constructs 39, 41, 25, 26, 23 and 42. Finally, the highest residual binding to the major repeats peptide was observed for Mosquirix®, RNA constructs 26, 8, 23, 2, 39 and 22.

### (8) FluoroSpot Assay

The ability of RNA constructs to elicit T-cell responses from whole splenocytes was also evaluated by a fluorospot assay. In this assay, the use of different fluorophore-coupled antibodies allows to simultaneously quantify the production of interferon gamma (IFNγ), interleukin (IL)-2 and (tumor necrosis factor alpha) TNFα by the splenocytes of immunized mice. In particular, provided polyribonucleotides can be assessed for their ability to induce production of antibodies responsive to recombinant PfCSP, MHC-I, and/or MHC-II peptides. In some embodiments, a polyribonucleotide is determined to induce a useful immune response if splenocytes from a subject (e.g., a mouse) immunized with such construct, following incubation with peptide(s) as described herein, exhibit T-cell secretion of one or more pro-inflammatory cytokines (e.g., IFN-γ, TNF-α, or IL-2) in a FluoroSpot Assay, as described herein.

FluoroSpot assays were performed with mouse IFN-γ/IL-2/TNF-α FluoroSpotPLUS kit according to the manufacturer's instructions (Mabtech). Frozen splenocytes from mice immunized with RNA constructs were thawed and washed twice in DPBS before being resuspended in culture medium (RPMI1640+10% heat-inactivated fetal calf serum (FCS)+1% non-essential amino acids (NEAA)+1% Sodium Pyruvate+1% HEPES+0.5% Penicillin/Streptomycin, +0.1% β-Mercaptoethanol).

After determining cell concentration for each sample using a cell counter, a total of 5×10⁵ splenocytes were added to each well and restimulated ex vivo overnight at 37° C. with the peptides indicated in Table 14. For RNA constructs 2, 6, 8, 22-26, 28-31, 33-35, 37, 39, 41, 42, and 45, a PfCSP-FL peptide pool (PfCSP-FL_pep), MHC-I peptide pool, MHC-II peptide pool or controls (negative control: gp70-AH1 (SPSYVYHQF; SEQ ID NO: 425), 4 μg/mL; positive control: concanavalin A, 2 μg/mL) were used. On the following day, anti-IFN-γ, anti-IL-2 and anti-TNF-α antibodies were added to the wells to detect production of these cytokines and then secondary antibodies conjugated with different fluorophores were added. Fluorescent spots were counted using a Mabtech IRIS FluoroSpot plate reader.

For RNA constructs 87, 88, 91, 100 and 104, a PfCSP-FL peptide pool (PfCSP-FL_pep) or controls (negative control: Trp1 (TAPDNLGYA; SEQ ID NO: 438), 4 μg/mL; positive control: concanavalin A, 2 μg/mL) were used. On the following day, anti-IFN-γ, anti-IL-2 and anti-TNF-α antibodies were added to the wells to detect production of these cytokines and then secondary antibodies conjugated with different fluorophores were added. Fluorescent spots were counted using a Mabtech IRIS FluoroSpot plate reader.

For RNA constructs 87, 88, 91, 100 and 104, CD4 and CD8 T cell responses were evaluated by MACS separating these cell populations prior to stimulation with PfCSP-FL_pep. Specifically, splenocytes were resuspended in MACS buffer, the biotin-labelled antibody cocktail was added to the cells, mixed and incubated for 5 minutes at 2-8° C. Then, MACS buffer was added again, followed by anti-biotin microbeads and an incubation for 10 minutes at 2-8° C. Columns were conditioned by adding MACS buffer to them and allowing the MACS buffer to go through the columns completely. Cell suspensions were then added to the columns, followed by a wash with MACS buffer, and the flow-through containing the population of interest (because a negative selection method is used) was collected in a tube. MACS-isolated CD4 and CD8 T cells were then counted for the whole splenocytes and 1×10⁵ cells were added per well of a fluorospot plate. Bone marrow-derived dendritic cells (5×10⁴ per well) were added to the CD4 and CD8 T cells for antigen presentation.

TABLE 14
Peptides used for splenocyte stimulation in the FluoroSpot Assay

| | AS | | | SEQ ID | |
|---|---|---|---|---|---|
| Designation | sequence | Presentation | Sequence | NO. | Comments |
| PICSP FL_pep | 1-397 | MHC-I and | MMRKLAILSVSSFLFVEALFQEYQCYGSSSNT | 395 | Pool of 65 |
| (overlapping | | MHC-II | RVLNELNYDNAGTNLYNELEMNYYGKQENW | | peptides (15 |
| peptides) | | | YSLKKNSRSLGENDDGNNEDNEKLRKPKHKK | | mer, |
| | | | LKQPADGNPDPNANPNVDPNANPNVDPNAN | | overlapping |
| | | | PNVDPNANPNANPNANPNANPNANPNANPN | | by 11) |
| | | | ANPNANPNANPNANPNANPNANPNANPNAN | | |
| | | | PNANPNANPNANPNVDPNANPNANPNANPN | | |
| | | | ANPNANPNANPNANPNANPNANPNANPNAN | | |
| | | | PNANPNANPNANPNANPNANPNANPNANPN | | |
| | | | KNNQGNGQGHNMPNDPNRNVDENANANSA | | |
| | | | VKNNNNEEPSDKHIKEYLNKIQNSLSTEWSP | | |
| | | | CSVTCGNGIQVRIKPGSANKPKDELDYANDI | | |
| | | | EKKICKMEKCSSVFNVVNSSIGLIMVLSFLFLN | | |
| CSPI-6 | 11-19 | MHC-I | SSFLFVEAL | 396 | Signal |
| | | | | | peptide |
| CSPI-8 | 63-72 | MHC-I | YSLKKNSRSL | 397 | N-terminal |
| | | | | | domain |
| CSPI-9 | 45-53 | MHC-I | TNLYNELEM | 398 | N-terminal |
| | | | | | domain |
| CSPI-10 | 350-358 | MHC-I | SANKPKDEL | 399 | C-terminal |
| | | | | | domain |
| CSPI-11 | 319-327 | MHC-I | YLNKIQNSL | 400 | C-terminal |
| | | | | | domain |
| CSPII-6 | 342-355 | MHC-II | IQVRIKPGSANKPK | 401 | C-terminal |
| | | | | | domain |
| CSPII-7 | 292-305 | MHC-II | NVDENANANSAVKN | 402 | C-terminal |
| | | | | | domain |
| CSPII-8 | 185-198 | MHC-II | NANPNANPNANPNV | 403 | major |
| | | | | | repeats |
| CSPII-9 | 293-305 | MHC-II | VDENANANSAVKN | 404 | C-terminal |
| | | | | | domain |
| CSPII-10 | 19-31 | MHC-II | LFQEYQCYGSSSN | 405 | N-terminal |
| | | | | | domain |

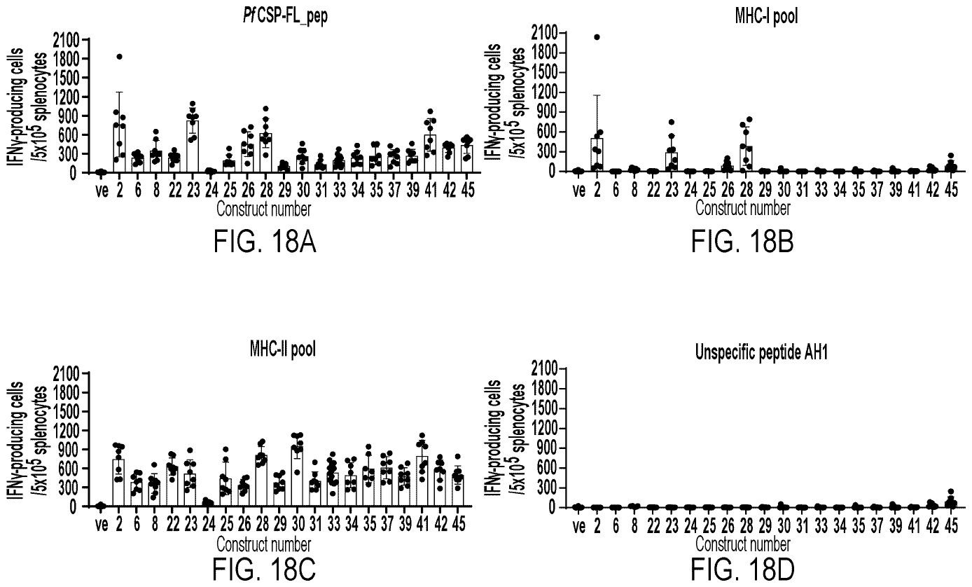

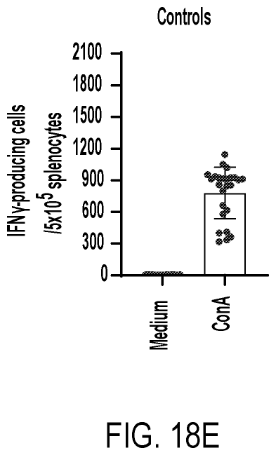

As shown in FIG. 18, upon stimulation with PfCSP-FL_pep and/or MHC-II-specific peptides, IFN-γ producing cells were detected in splenocytes from mice immunized with tested RNA constructs except for RNA construct 24. Splenocytes from mice immunized with tested RNA constructs (except for RNA construct 24) had an average of at least about 100 IFN-γ-producing cells per 5×10⁵ splenocytes after stimulation with PfCSP-FL_pep. Splenocytes from mice immunized with RNA construct 2, 23, 28, or 41 had the highest average with at least about 600 IFN-γ-producing cells per 5×10⁵ splenocytes after stimulation with PfCSP-FL_pep. Splenocytes from mice immunized with tested RNA constructs (except for RNA construct 24) had an average of at least about 300 IFN-γ-producing cells per 5×10⁵ splenocytes after stimulation with MHC-II specific peptides. Splenocytes from mice immunized with RNA constructs 2, 28, 30, and 41 had the highest average with at least about 600 IFN-γ-producing cells per 5×10⁵ splenocytes after stimulation with MHC-II specific peptides. In contrast, upon stimulation with MHC-I specific peptides, splenocytes from mice immunized only with tested RNA construct 2, 23, or 28 exhibited activation of T-cell IFN-γ secretion (with an average of at least about 300 IFN-γ-producing cells per 5×10⁵ splenocytes).

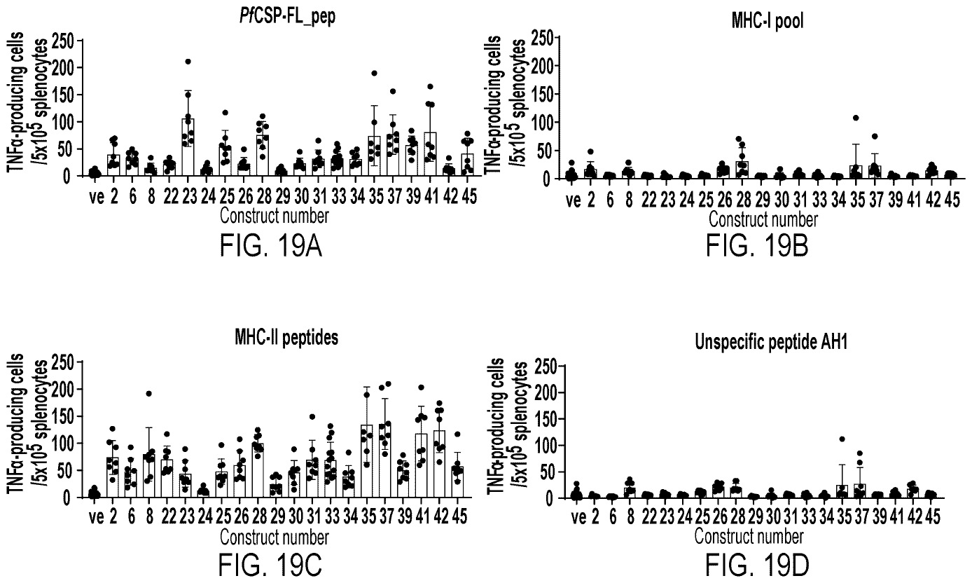

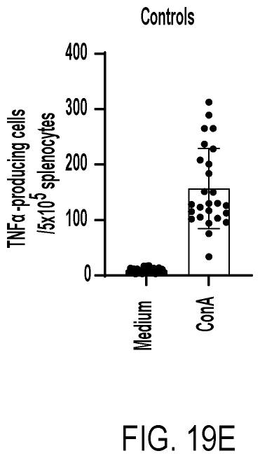

As shown in FIG. 19, TNF-α producing cells were detected in splenocytes from mice immunized with all RNA constructs, upon stimulation with PfCSP-FL_pep or MHC-II-specific peptides, but not with MHC-I specific peptides. Splenocytes from mice immunized with tested RNA construct 23, 25, 28, 35, 37, 39, or 41 had an average of at least 50 TNF-α-producing cells per 5×10⁵ splenocytes after stimulation with PfCSP-FL_pep. Splenocytes from mice immunized with RNA construct 23 had the highest average with about 100 TNF-α-producing cells per 5×10⁵ splenocytes after stimulation with PfCSP-FL_pep. Splenocytes from mice immunized with tested RNA constructs (except for RNA constructs 24, 29, and 34) had an average of at least about 50 TNF-α-producing cells per 5×10⁵ splenocytes after stimulation with MHC-II specific peptides. Splenocytes from mice immunized with RNA construct 28, 35, 37, 41, or 42 had the highest average with at least about 100 TNF-α-producing cells per 5×10⁵ splenocytes after stimulation with MHC-II specific peptides. In contrast, upon stimulation with MHC-I specific peptides, only splenocytes from mice immunized with tested RNA construct 2, 8, 26, 28, 35, 37, or 42 exhibited activation of T-cell TNF-α secretion.

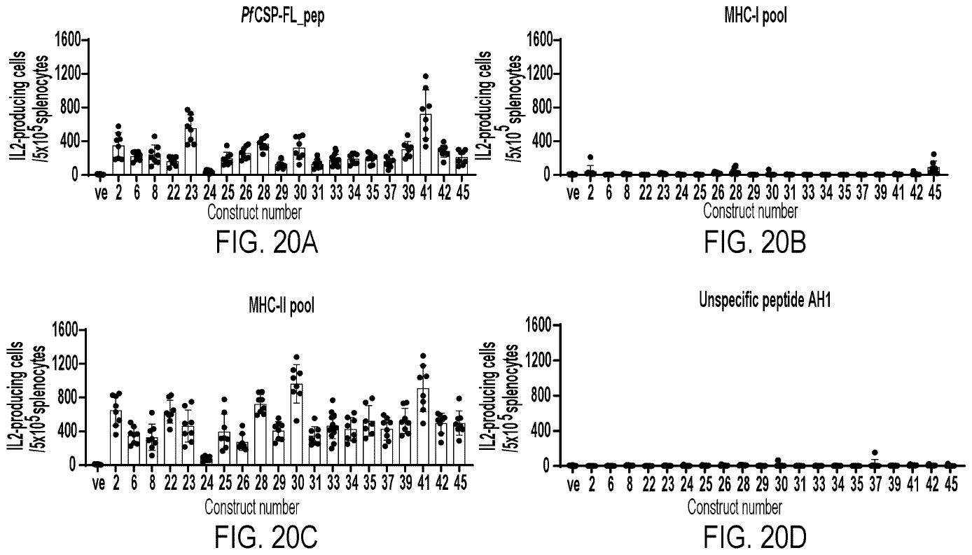

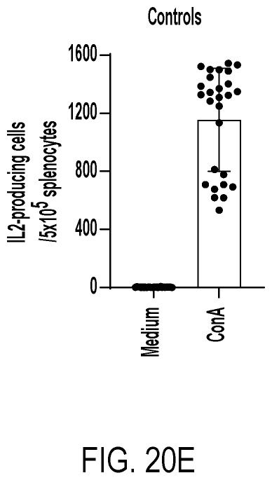

As shown in FIG. 20, upon stimulation with PfCSP-FL_pep and/or MHC-II-specific peptides, IL-2 producing cells were detected in splenocytes from mice immunized with most RNA constructs. Splenocytes from mice immunized with tested RNA constructs (except RNA construct 24) had an average of at least about 100 IL-2-producing cells per 5×10⁵ splenocytes after stimulation with PfCSP-FL_pep. Splenocytes from mice immunized with RNA construct 2, 23, 28, or 41 had the highest average (about 350 to about 800 TNF-α-producing cells per 5×10⁵ splenocytes) after stimulation with PfCSP-FL_pep. Splenocytes from mice immunized with tested RNA constructs (except for RNA construct 24) had an average of at least about 300 IL-2-producing cells per 5×10⁵ splenocytes after stimulation with MHC-II specific peptides. Splenocytes from mice immunized with RNA construct 30 or 41 had the highest average with at least about 800 TNF-α-producing cells per 5×10⁵ splenocytes after stimulation with MHC-II specific peptides. There was almost no response upon stimulation with MHC-I specific peptides.

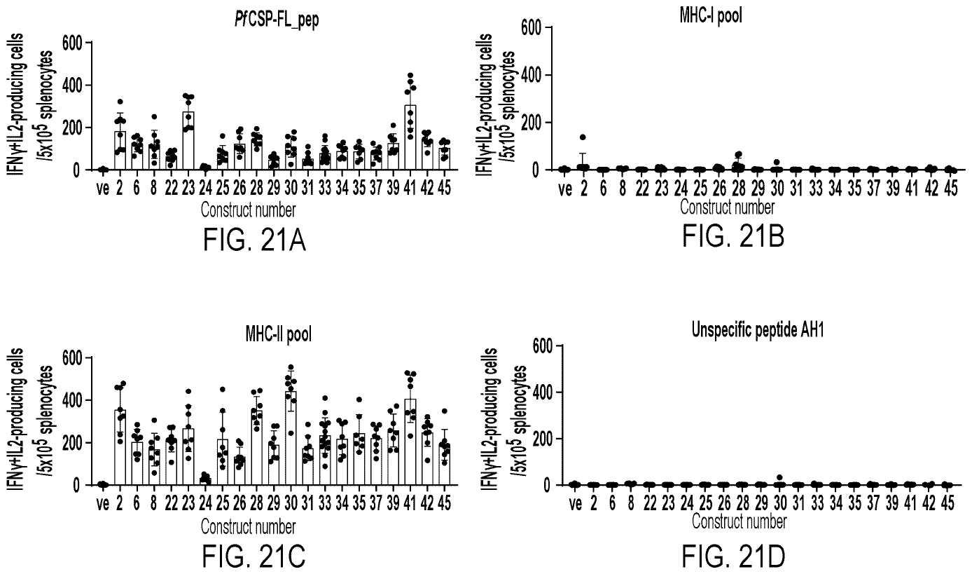

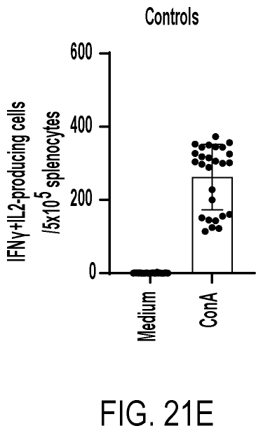

As shown in FIG. 21, T-cells secreting both IL-2 and IFN-γ were detected in splenocytes from mice immunized with most RNA constructs upon stimulation with PfCSP-FL_pep or MHC-II specific peptides, but not MHC-I specific peptides. Splenocytes from mice immunized with tested RNA constructs (except RNA construct 24) had an average of at least about 50 IL-2 and IFN-γ-producing cells per 5×10⁵ splenocytes after stimulation with PfCSP-FL_pep. Splenocytes from mice immunized with RNA construct 23 or 41 had the highest average (at least about 300 IL-2/IFN-γ-producing cells per 5×10⁵ splenocytes) after stimulation with PfCSP-FL_pep. Splenocytes from mice immunized with tested RNA constructs (except for RNA construct 24) had an average of at least about 150 IL-2/IFN-γ-producing cells per 5×10⁵ splenocytes after stimulation with MHC-II specific peptides. Splenocytes from mice immunized with RNA construct 2, 28, 30 or 41 had the highest average with at least about 350 IL-2/IFN-γ-producing cells per 5×10⁵ splenocytes after stimulation with MHC-II specific peptides. There was almost no response upon stimulation with MHC-I specific peptides.

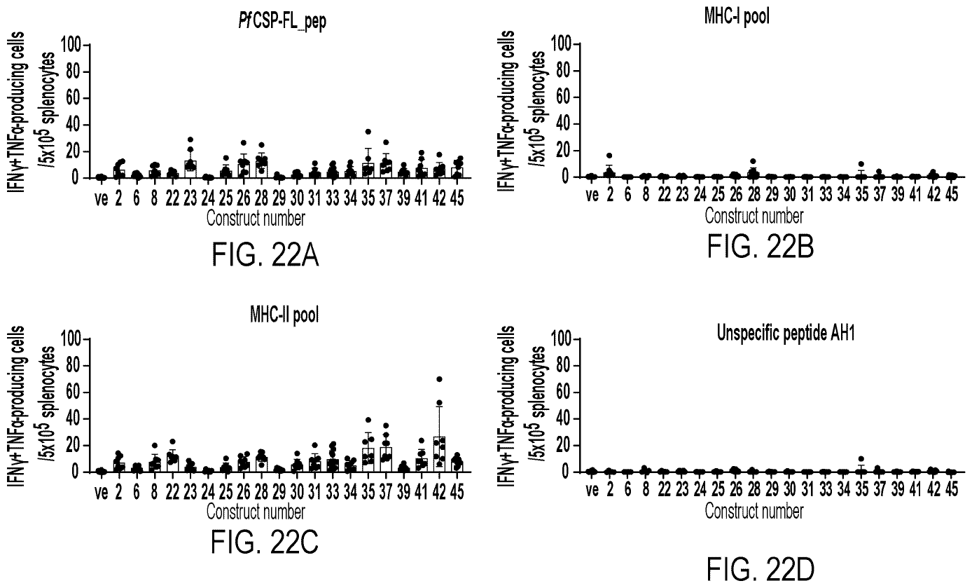

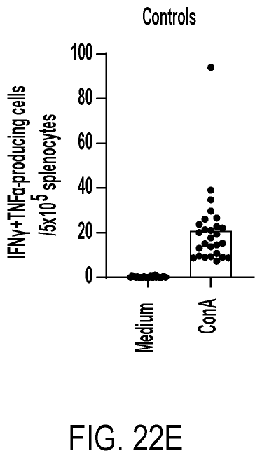

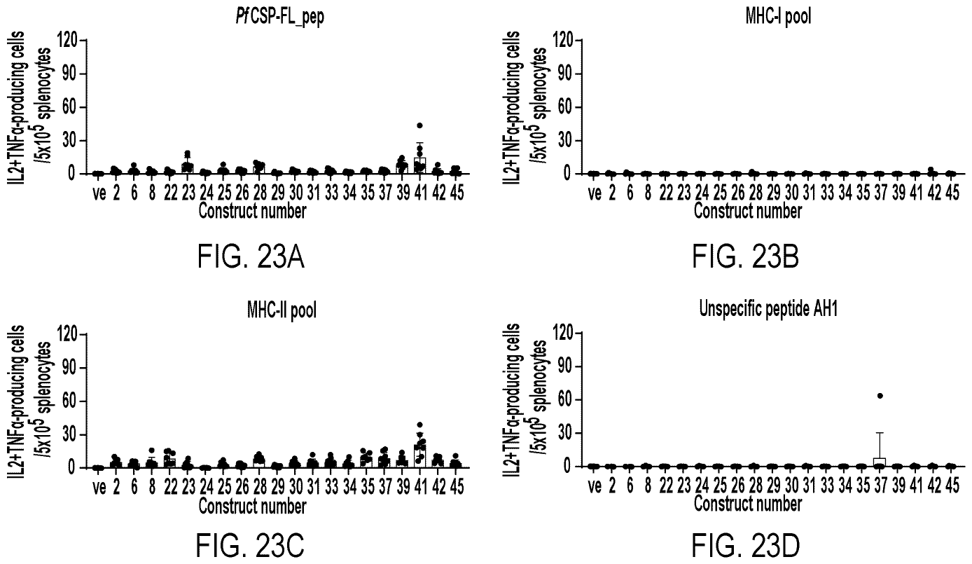

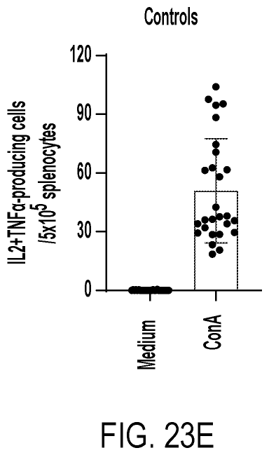

As shown in FIG. 22 and FIG. 23, the number of T-cells secreting both IFN-γ and TNF-α, or secreting both IL-2 and TNF-α, was low in splenocytes from mice immunized with tested RNA constructs upon stimulation with PfCSP-FL_pep, MHC-II specific peptides, or MHC-I specific peptides. For example, splenocytes from mice immunized with tested RNA constructs had an average of less than about 20 IFN-γ/TNF-α-producing cells or IL-2/TNF-α-producing cells, per 5×10⁵ splenocytes, after stimulation with PfCSP-FL_pep or after stimulation with MHC-II specific peptides.

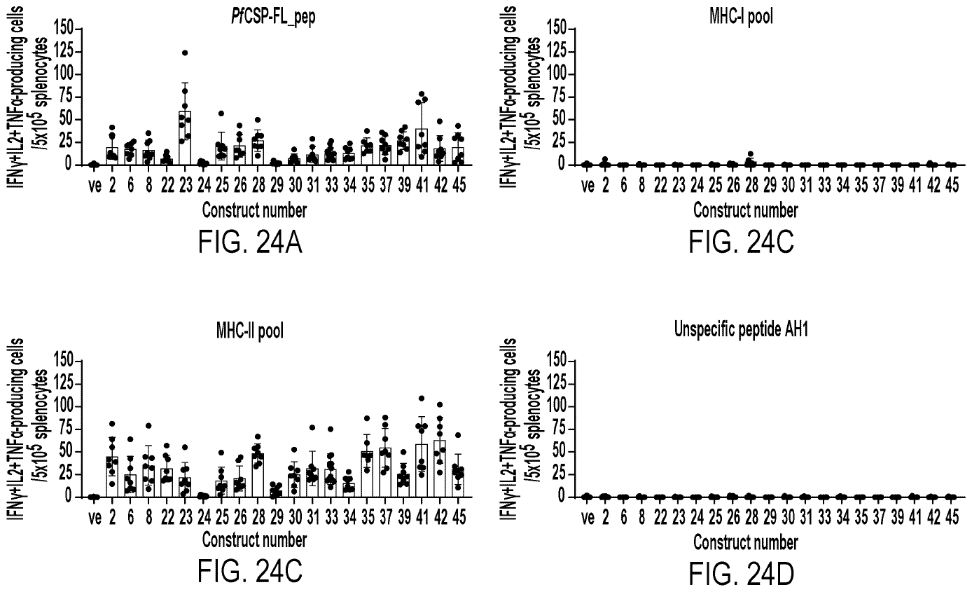

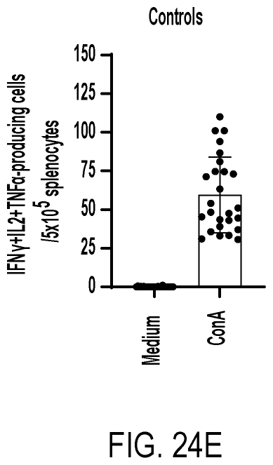

As shown in FIG. 24, T-cells secreting IFN-γ, IL-2, and TNF-α were detected in splenocytes from mice immunized with most RNA constructs upon stimulation with PfCSP-FL_pep or MHC-II specific peptides, but not upon stimulation with MHC-I specific peptides. Splenocytes from mice immunized with tested RNA constructs (except RNA construct 24, 29, and 30) had an average of at least about 10 IFN-γ/IL-2/TNF-α-producing cells per 5×10⁵ splenocytes after stimulation with PfCSP-FL_pep. Splenocytes from mice immunized with RNA construct 23 or 41 had the highest average (about 55 and about 40 IFN-γ/IL-2/TNF-α-producing cells per 5×10⁵ splenocytes, respectively) after stimulation with PfCSP-FL_pep. Splenocytes from mice immunized with tested RNA constructs (except for RNA constructs 24 and 29) had an average of at least about 25 IFN-γ/IL-2/TNF-α-producing cells per 5×10⁵ splenocytes after stimulation with MHC-II specific peptides. Splenocytes from mice immunized with RNA construct 2, 28, 35, 37, 41, or 42 had the highest average with at least about 50 IFN-γ/IL-2/TNF-α-producing cells per 5×10⁵ splenocytes after stimulation with MHC-II specific peptides. There was almost no response upon stimulation with MHC-I specific peptides.

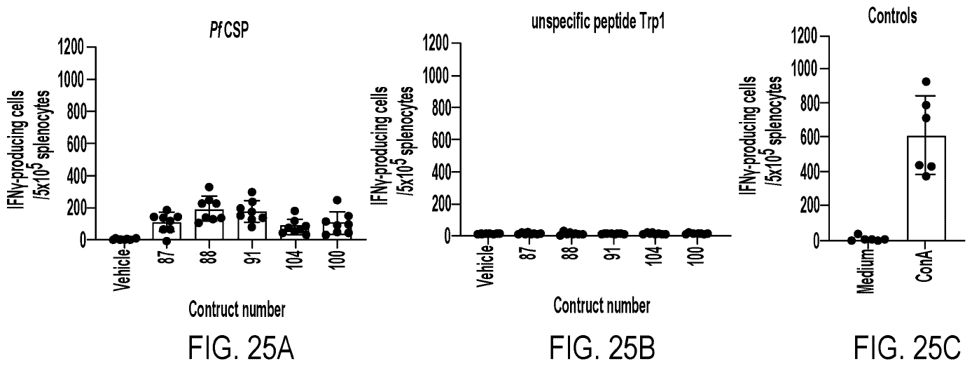

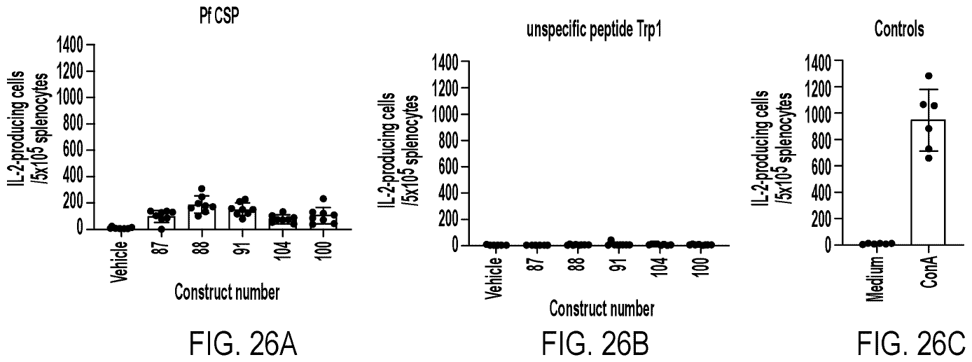

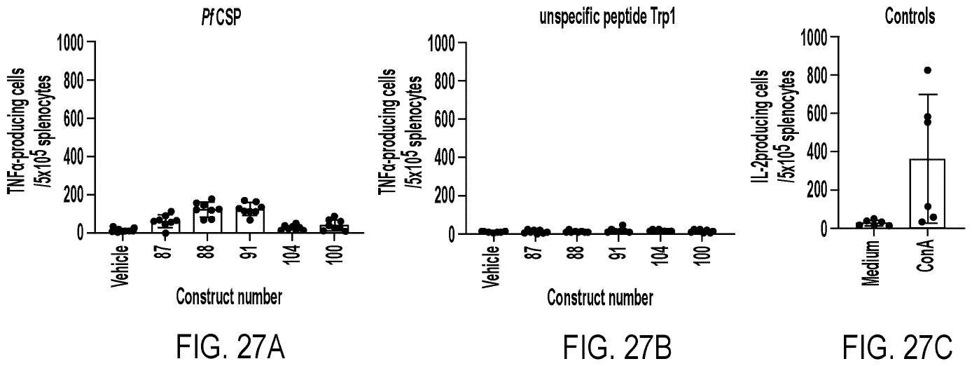

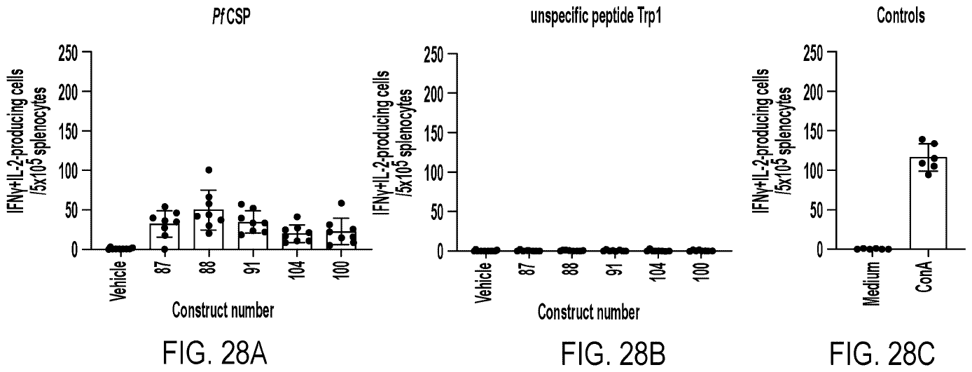

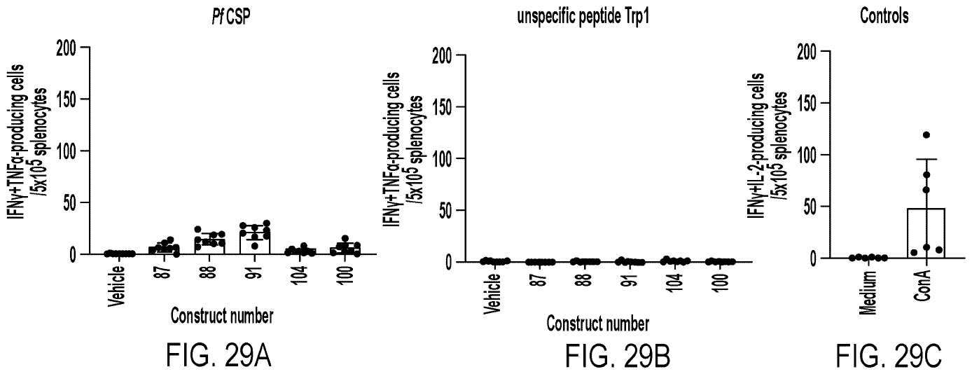

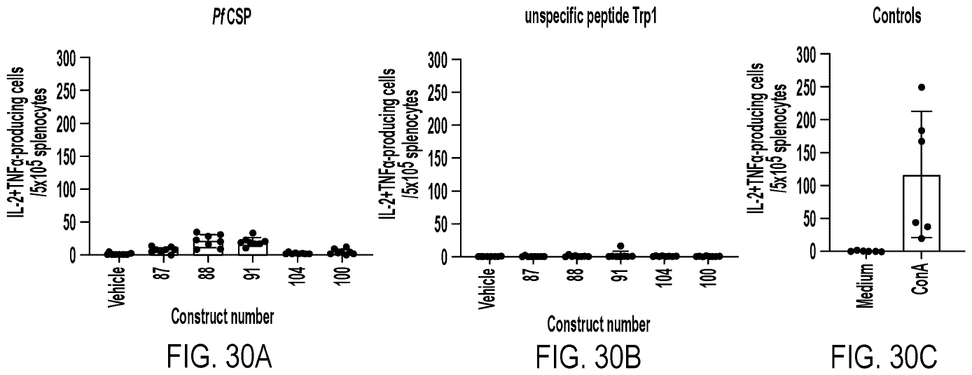

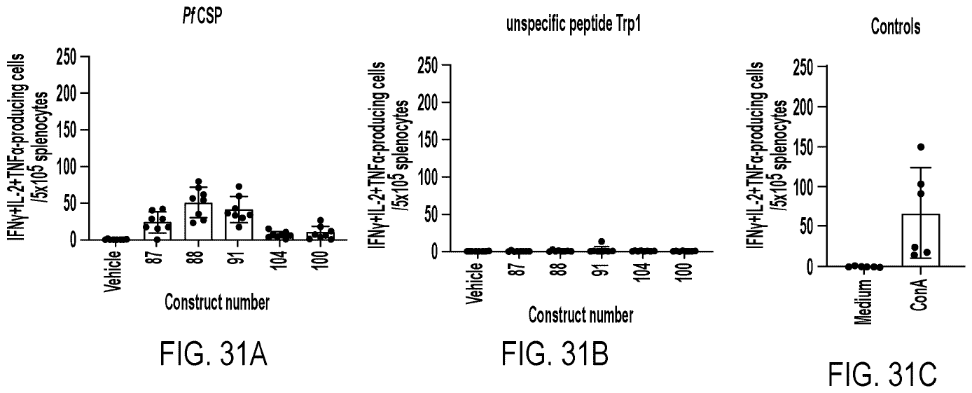

As shown in FIGS. 25-31, the responses observed for RNA constructs 100 and 104 were lower than those observed for RNA constructs 87, 88, and 91. A response was not observed from splenocytes from mice injected with vehicle alone. Incubation of the splenocytes from immunized mice with the unspecific peptide Trp1 or with culture medium only, also did not elicit a response from the cells.

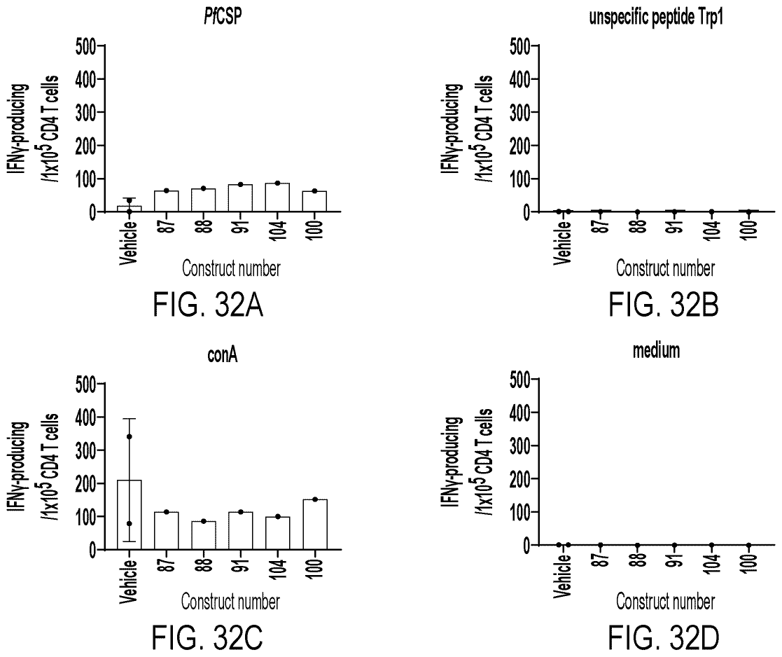

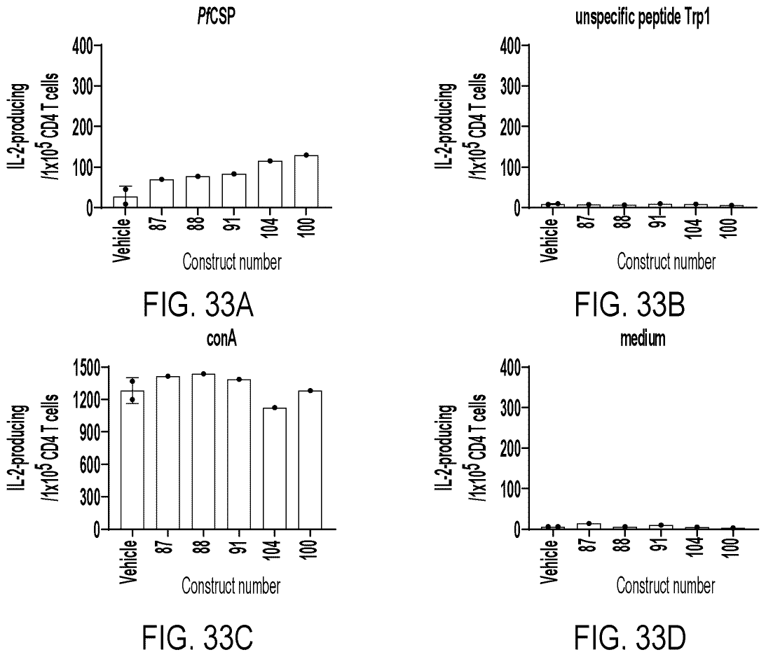

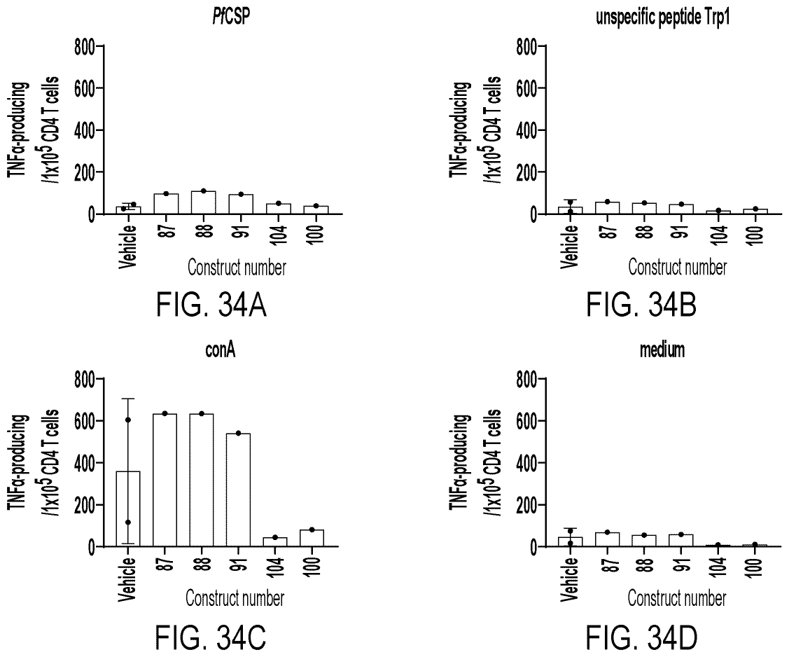

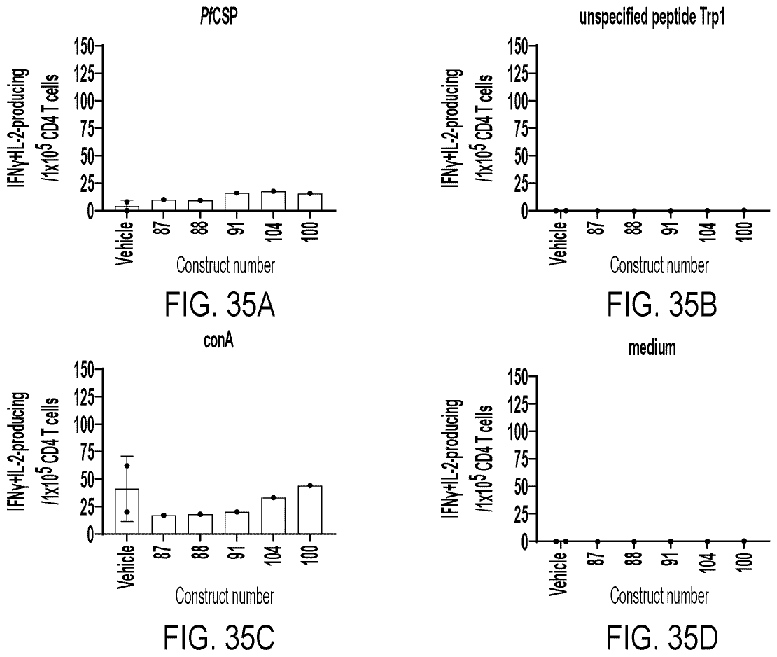

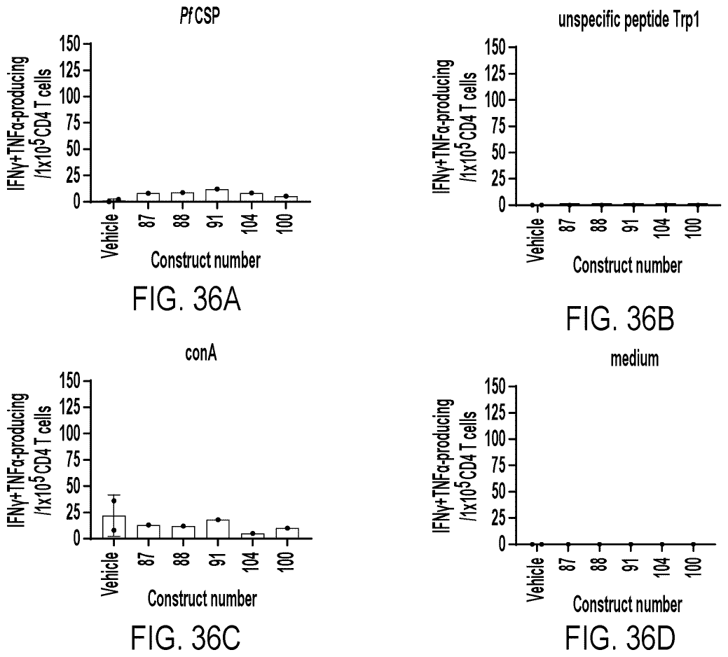

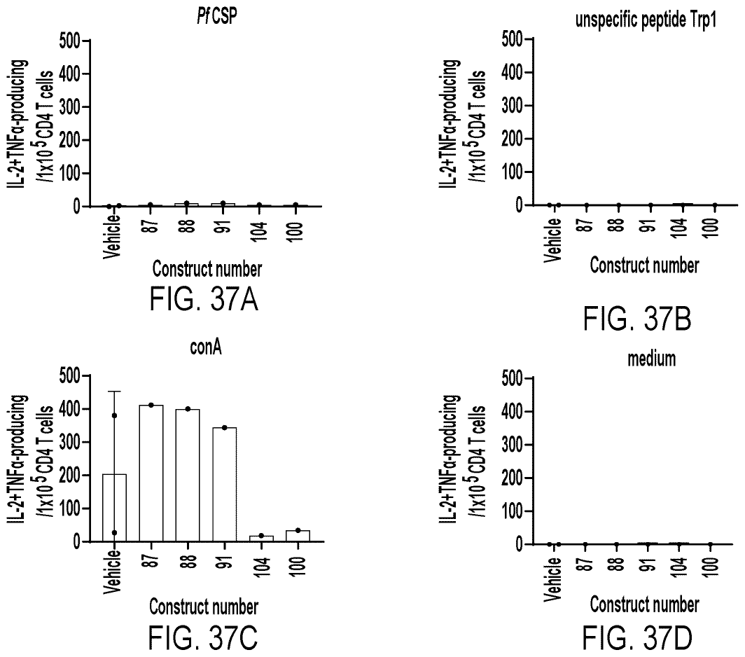

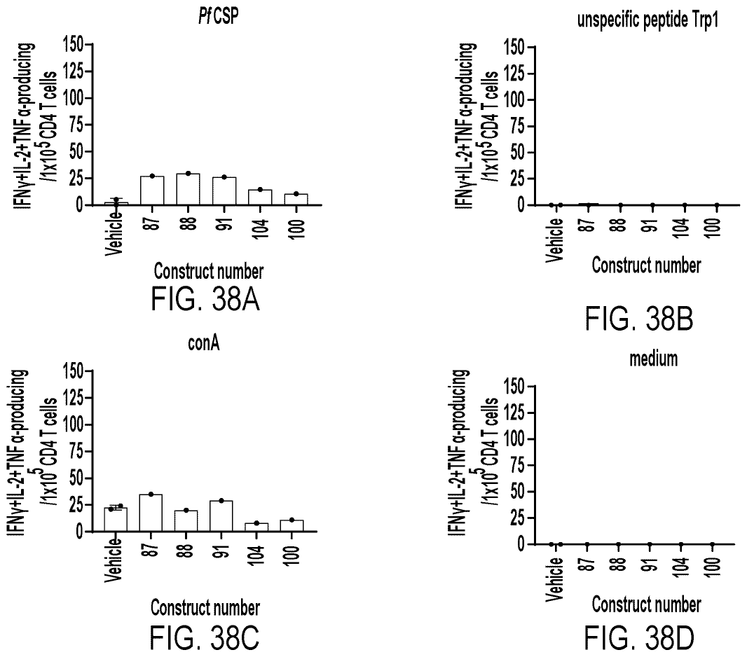

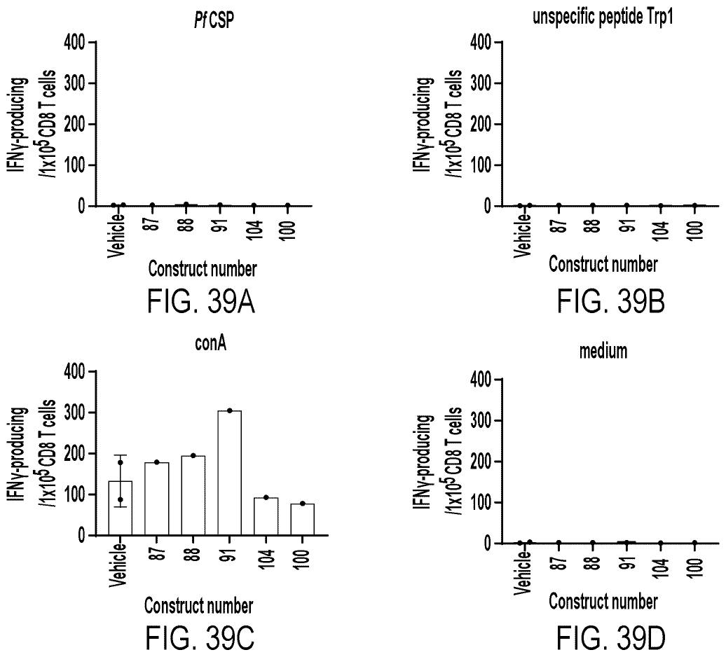

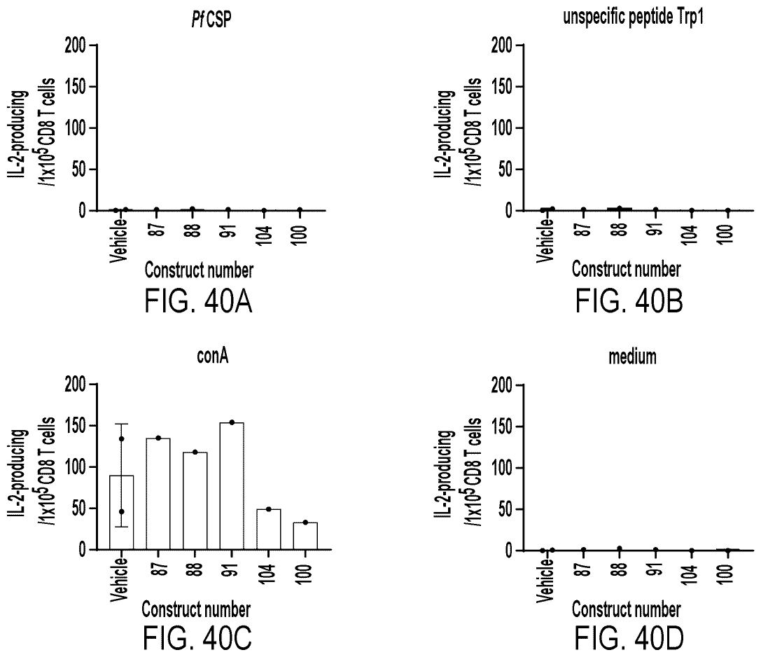

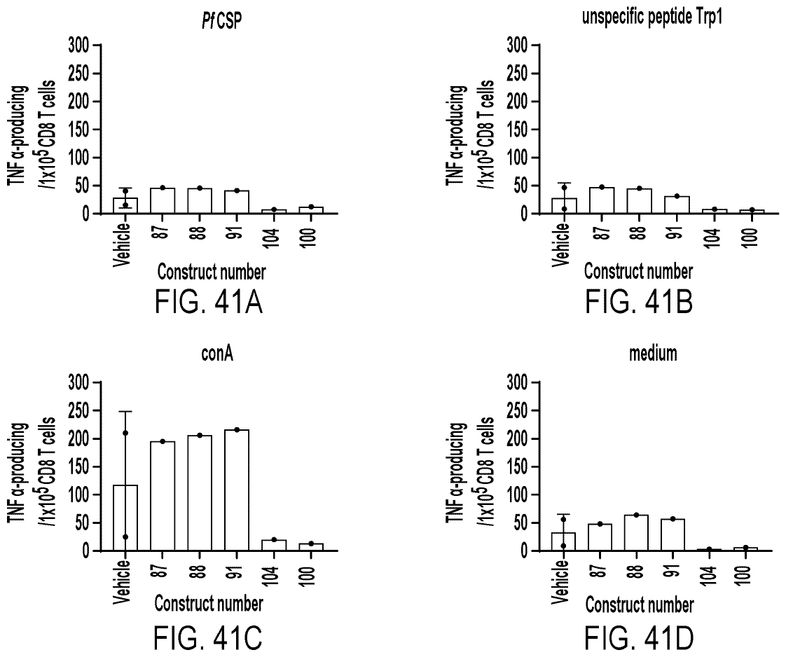

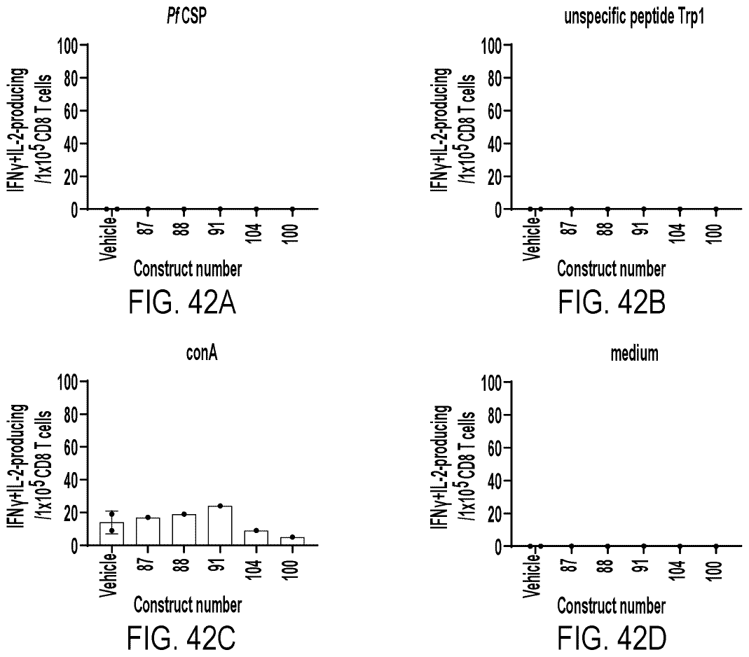

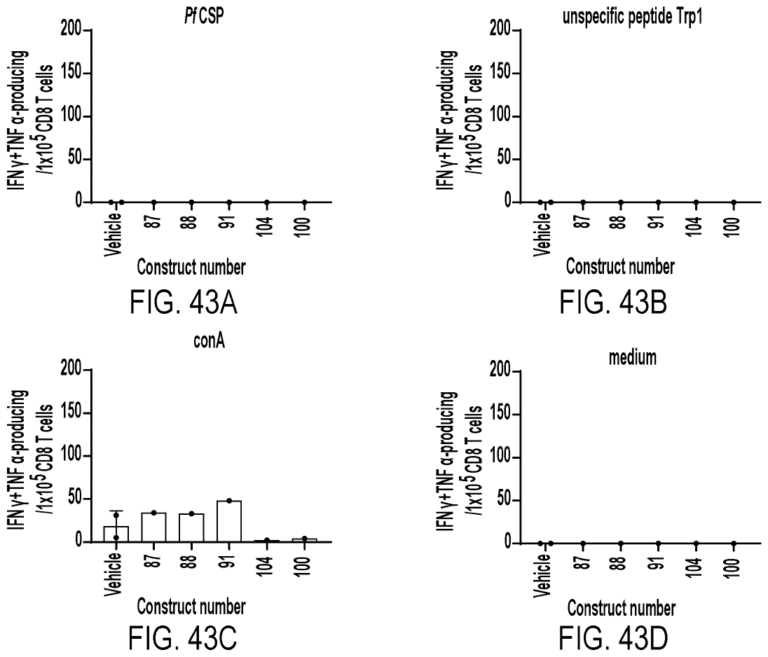

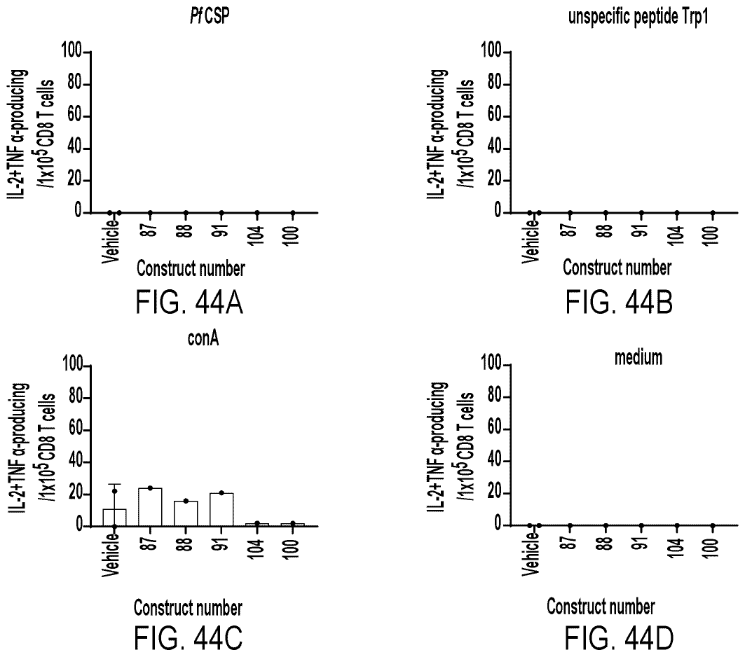

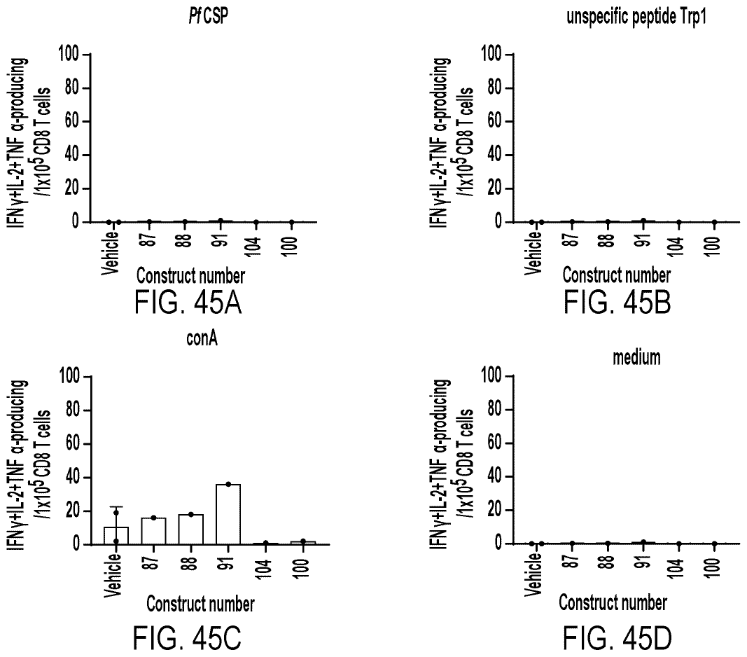

The ability of RNA constructs 87, 88, 91, 100 and 104 to elicit T-cell responses from isolated CD4 and CD8 T cells was evaluated by a fluorospot assay, after MACS separation of these specific cell populations. As shown in FIGS. 32-45, upon incubation with overlapping peptides spanning the PfCSP-FL protein, CD4 T cells from mice immunized with the different constructs produced pro-inflammatory cytokines, such as IFNγ, IL-2 and TNFα, alone or in different combinations (FIGS. 32-38). A similar CD4 T cell response was observed for all the RNA constructs. On the other hand, no response was observed from CD8 T cells (FIGS. 39-45). A response was also not observed for the vehicle group or upon incubation with the unspecific peptide Trp1 or with culture medium only.

Thus, the present Example demonstrates that certain polyribonucleotides effectively induce an immune response characterized by activation of T-cells secreting one or more pro-inflammatory cytokines (e.g., IFN-γ, TNF-α, and/or IL-2), e.g., assessed using a fluorospot assay. All except one RNA construct induced an immune response characterized by activation of T-cells secreting against at least one pro-inflammatory cytokines (e.g., IFN-γ, TNF-α, or IL-2).

In view of the results, the peptides within the peptide pools were analyzed and it was found that the data generated upon incubation with MHC-I peptide pool is not representative of the ability of each construct to stimulate CD8 T cell responses. After analysis of the individual peptides, only 2 peptides present in the N-terminus of CSP have shown ability to stimulate cells. Therefore, only constructs with N-terminus had detectable responses. For all other constructs, absence of a response with MHC-I peptide pool did not mean the construct was unable to elicit a CD8 T cell response.

### (9) Sporozolte Immunofluorescence Assay

Provided polyribonucleotides can be assessed for their ability to induce production of antibodies that may bind to native PfCSP on PfCSP-expressing Plasmodium berghei (PbPf) sporozoites. In some embodiments, a provided polyribonucleotide is determined to induce a useful immune response if serum from a subject (e.g., a mouse) immunized with such polyribonucleotide is shown to bind to native PfCSP on PbPf sporozoites in a sporozoite immunofluorescence assay, as described herein.

In the sporozoite immunofluorescence assay, sporozoites were fixed with 2% formaldehyde in PBS for 20 min at room temperature. Then, sporozoites were washed on a spin-X centrifuge tube filter 0.45 μm Cellulose Acetate and maintained at 4° C. until further experiments. 4,000 PfCSP-expressing P. berghei sporozoites per sample were prepared in 1% bovine serum albumin (BSA)-PBS in a total volume of 5 μL. Sporozoites were mixed with 5 μL serum (from mice immunized with either a positive control (e.g., anti-PfCSP 2A10 mAb IV) or with RNA constructs (as described in Example 1)) diluted in 1% BSA-PBS (10-fold serial dilutions, from 1:103 to 1:108). Samples were incubated overnight at 4° C. in a humid chamber in the dark. The day after, samples were incubated with Alexa 647 donkey anti-mouse IgG (H+L) in 1% BSA-PBS (final concentration of 20 μg/mL) for 30 min at 4° C. in the dark. Samples were 11-fold diluted with cold PBS prior to acquisition by flow cytometry. The median antigen presenting cells (APC) intensity of the sporozoite population at different dilutions was calculated and log transformed. The cut-off values were determined according to the methods described (Frey et al. 1998), and used to interpolate the titer values from a linear regression.

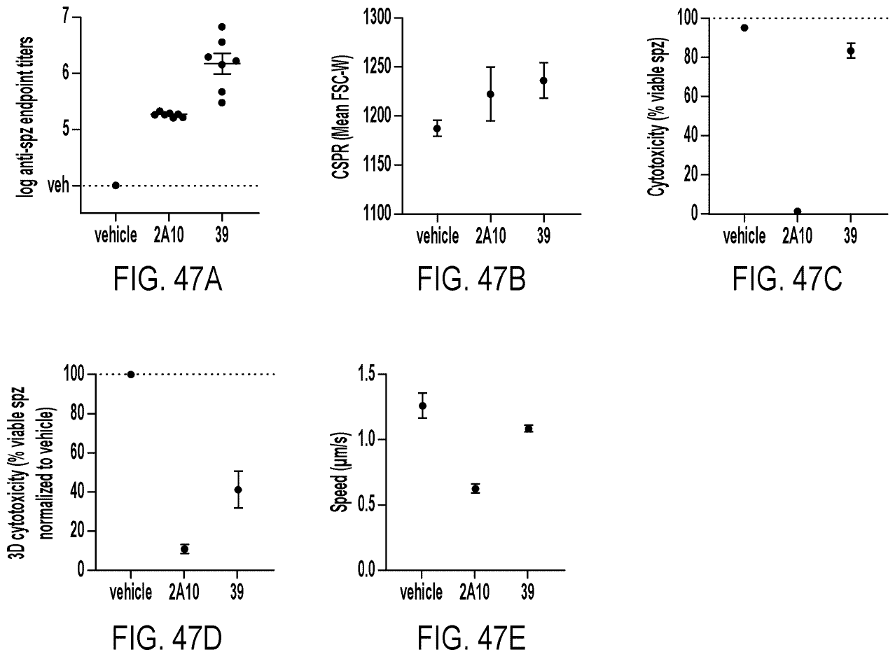

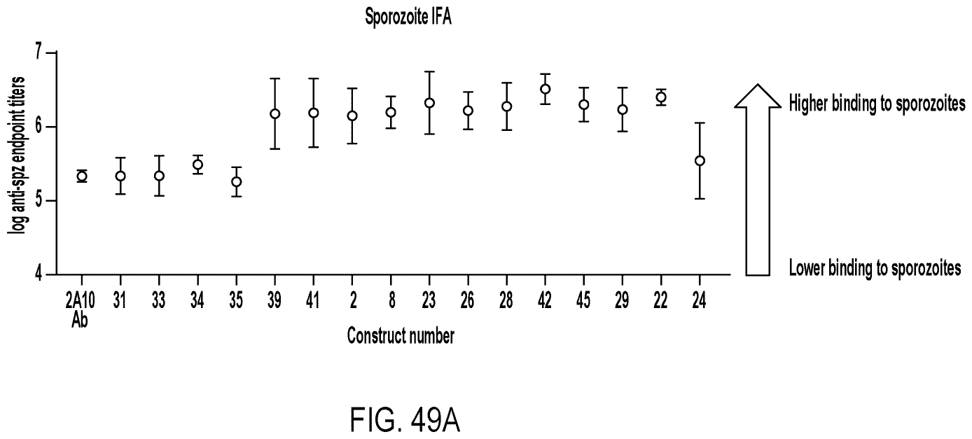

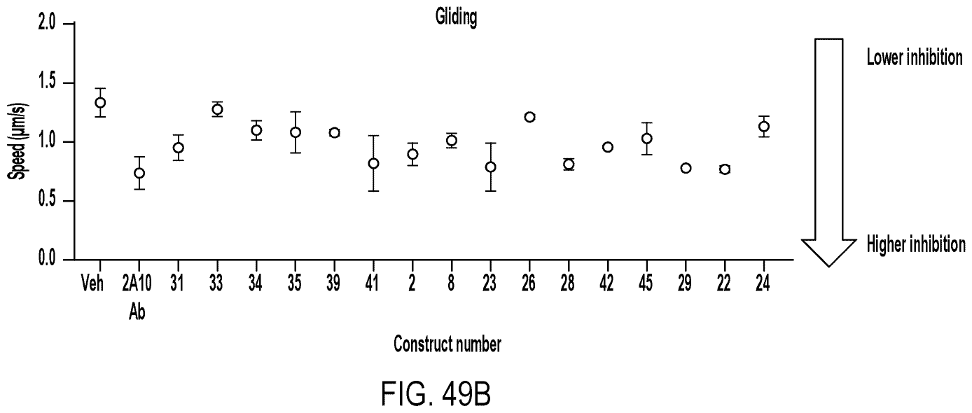

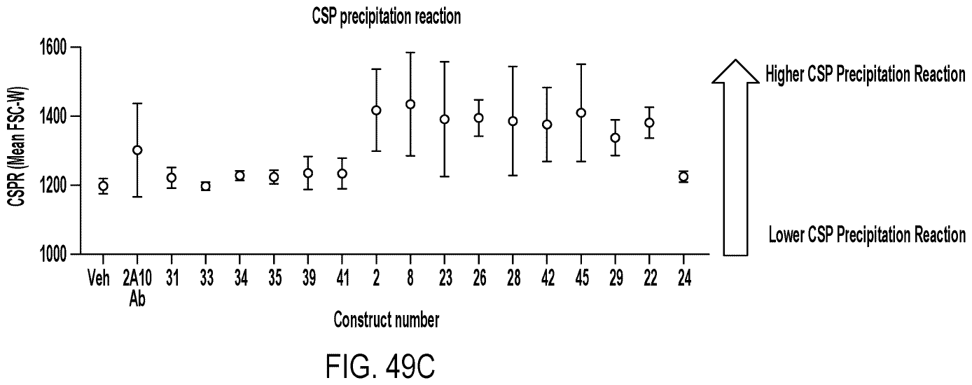

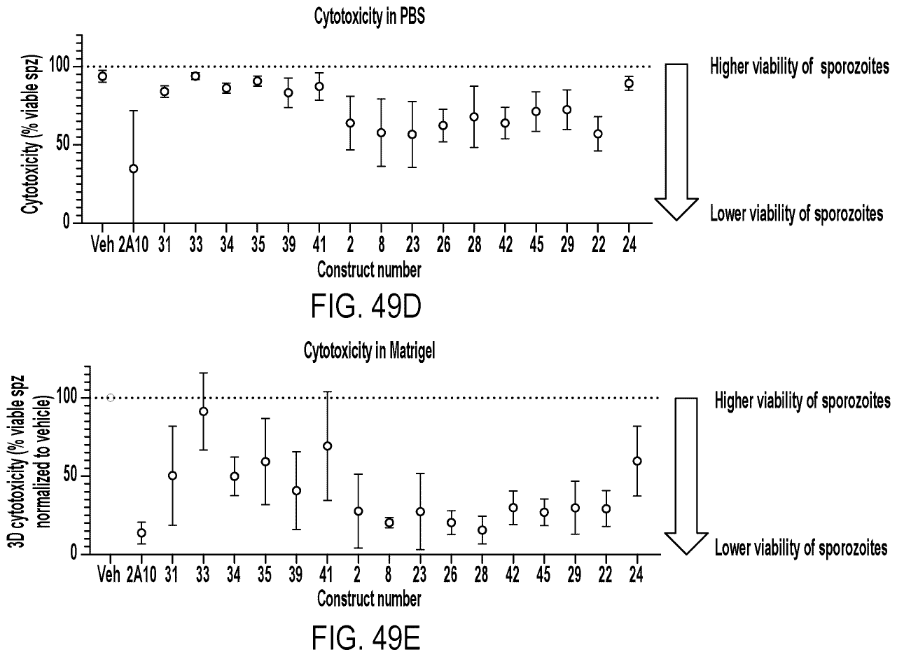

As shown in FIG. 47A, RNA construct 39 induced high levels of antibodies to native PfCSP on PbPf sporozites (exhibiting a median reciprocal end titer of at least about 106 (at least about 6 after LOG transformation)). Sera from mice immunized with construct 39, at different dilutions, were more variable in their ability to induce antibodies to native PfCSP on PbPf sporozites (exhibiting a median reciprocal end titer of least about 105 to 107 (at least about 5 to 7 after LOG transformation)), as compared to sera from mice immunized with anti-PfCSP 2A10 mAb IV, at different dilutions (exhibiting a median reciprocal end titer of least about LOG 105 to LOG 106 (at least about 5 to 6 after LOG transformation)). Additionally, as shown in FIG. 49A, higher titers of PfCSP-binding antibodies were observed for mice immunized with RNA constructs 39, 41, 2, 8, 23, 26, 28, 42, 45, 29 and 22 when compared to mice injected with the remaining RNA constructs or with 100 μg of 2A10 monoclonal antibody.

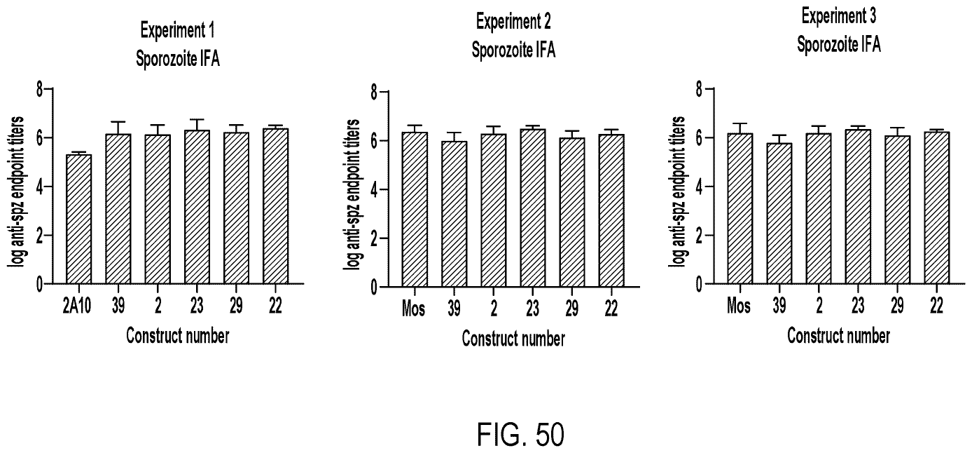

Sporozoite IFA experiments were also performed to assess RNA constructs 2, 22, 23, 29, and 39. As shown in FIG. 50, in Experiment 1, sera from all the tested constructs showed a higher binding to PfCSP on the sporozoites than the sera from the 2A₁₀-injected control. In Experiments 2 and 3, a similar binding was observed for sera from mice immunized with the RNA constructs and sera from mice immunized with Mosquirix®.

Thus, the present Example demonstrates that provided RNA constructs provided herein effectively induced an immune response characterized by production of antibodies that bind to native PfCSP on PbPf sporozoites as assessed using a sporozoite immunofluorescence assay. For example, sera from mice immunized with RNA constructs 39, 41, 2, 8, 23, 26, 28, 42, 45, 29 and 22 were shown to have induced a stronger antibody response to native PfCSP on PbPf sporozites, as compared to sera from mice immunized with anti-PfCSP 2A10 mAb IV (exhibiting a median reciprocal end titer of least about LOG 105 to LOG 106.

### (10) Circumsporozoite Precipitation Reaction (CSPR) Assay

Provided polyribonucleotides can be assessed for their ability to induce production of antibodies that can elicit a CSP reaction on viable sporozoites. In some embodiments, a provided polyribonucleotide is determined to induce a useful immune response if serum from a subject (e.g., a mouse) immunized with such construct is shown to induce precipitation of CSP on viable sporozoites as measured by flow cytometry, as described herein.

In the circumsporozoite precipitation reaction (CSPR) assay, 12,000 PbPf sporozoites were incubated at 37° C. for 45 min with 17% serum (from mice immunized with either a positive control (e.g., anti-PfCSP 2A10 mAb IV) or with RNA constructs (as described in Example 1). Samples were next placed on ice, incubated for 10 min with 5 μg/mL propidium iodide and diluted 21 times with cold PBS prior to acquisition by flow cytometry. CSPR was measured by the estimated sporozoite length assessed by the mean of forward-scatter width (FSC—W). Data were analyzed using the CytExpert 2.0 software, which is incorporated herein by reference in its entirety.

As shown in FIG. 47B, sera from mice immunized with RNA construct 39 increased circumsporozoite precipitation (exhibited by a mean FSC—W of at least about 1230), as compared to sera from mice administered vehicle (exhibited by a mean FSC—W of at least about 1175). Sera from mice immunized with anti-PfCSP 2A10 mAb had a similar effect on circumsporozoite precipitation as compared to sera from mice immunized with RNA construct 39 IV (exhibited by a mean FSC—W of at least about 1220). Further, as shown in FIG. 49C, serum from mice immunized with RNA constructs 2, 8, 23, 26, 28, 42, 45, 29 or 22 elicited higher CSPR than serum from mice injected with the remaining RNA constructs, 2A10 or vehicle.

CSPR was also assessed for RNA constructs 2, 22, 23, 29, and 39 across three experiments. In Experiment 1, a higher CSPR was observed for RNA constructs 2, 23, 29 and 22 when compared to serum from mice injected with the vehicle. Mice injected with 2A10 also had higher CSPR. In Experiments 2 and 3, the highest CSPR was observed for RNA constructs 2 and 23, and to a similar level as that observed for Mosquirix®.

Thus, the present Example demonstrates that RNA constructs provided herein effectively induced an immune response characterized by production of antibodies that elicit CSP precipitation on viable sporozoites e.g., assessed using a circumsporozoite precipitation reaction (CSPR) Assay. For example, sera from mice immunized with RNA constructs 2, 8, 23, 26, 28, 42, 45, 29 and 22 were shown to have induced a stronger response as compared to sera from mice immunized with anti-PfCSP 2A10 mAb.

### (11) Cytotoxicity

Provided polyribonucleotides can be assessed for their ability to induce production of antibodies with an inhibitory effect on sporozoite viability. In some embodiments, a provided polyribonucleotide is determined to induce a useful immune response if serum from a subject (e.g., a mouse) immunized with such construct is shown to reduce viable sporozoites in a cytotoxicity assay, as described herein.

In the cytotoxicity assay, 12,000 PbPf sporozoites were incubated at 37° C. for 45 min with 17% serum (from mice immunized with either a positive control (e.g., anti-PfCSP 2A10 mAb IV) or RNA constructs (as described in Example 1). Samples were next placed on ice, incubated for 10 min with 5 μg/mL propidium iodide and diluted 21 times with cold PBS prior to acquisition by flow cytometry. CSPR was measured by the estimated sporozoite length assessed by the mean of FSC-Width. Viability was defined as the percentage of GFP+PI⁻ sporozoites to the sum of GFP⁺PI⁻, GFP⁻PI+ and GFP⁺PI⁺ sporozoites. Data were analyzed using the CytExpert 2.0 software.

In a following experiment, PbPf sporozoites were mixed on ice with Corning® Matrigel® matrix in a 1:5 ratio. For each condition, the mix was divided into 5 μL reactions containing 12,000 sporozoites. Following polymerization at 37° C. for 5 min, 1 μL serum (17% final concentration; from mice immunized with either a positive control (e.g., anti-PfCSP 2A10 mAb IV) or RNA constructs (as described in Example 1) was added. After 45 min at 37° C., samples were placed on ice for 10 min to allow depolymerization, followed by an incubation of 10 min with 5 μg/mL propidium iodide and finally diluted 21 times with cold PBS prior to acquisition by flow cytometry. The number of GFP⁺PI sporozoites from the negative control group was used to normalize the sporozoite recovery across samples. Viability was defined as the percentage of GFP⁺PI sporozoites compared to the negative control. Data was analyzed using the CytExpert 2.0 software.

As demonstrated, in FIG. 47C, as well as FIG. 49D and FIG. 49E, sera from mice immunized with various RNA constructs reduced viable sporozoites in suspension (exhibited by a mean of at least about 80% viable sporozoite) as compared to sera from mice administered a vehicle (exhibited by a mean of at least about 95% viable sporozoite). Incubation with serum from mice immunized with RNA constructs 2, 8, 23, 26, 28, 42, 45, 29 and 22 lead to a higher decrease in the viability of the sporozoites, as also observed for serum from mice passively immunized with 2A10 monoclonal antibody. Further, as demonstrated in FIG. 47D, and FIG. 49D and FIG. 49E, sera from mice immunized with certain RNA constructs greatly reduced viable sporozoites in 3D (i.e., Matrigel) (exhibited by a mean of at least about 40% viable sporozoite). Sera from mice immunized with anti-PfCSP 2A10 mAb IV greatly reduced viable sporozoites in suspension (exhibited by a mean of at least about 0% viable sporozoite) and in 3D (i.e., Matrigel) (exhibited by a mean of at least about 10% viable sporozoite). The decrease in viability of the sporozoites was more pronounced when the incubation is done in Matrigel than when it was done in PBS.

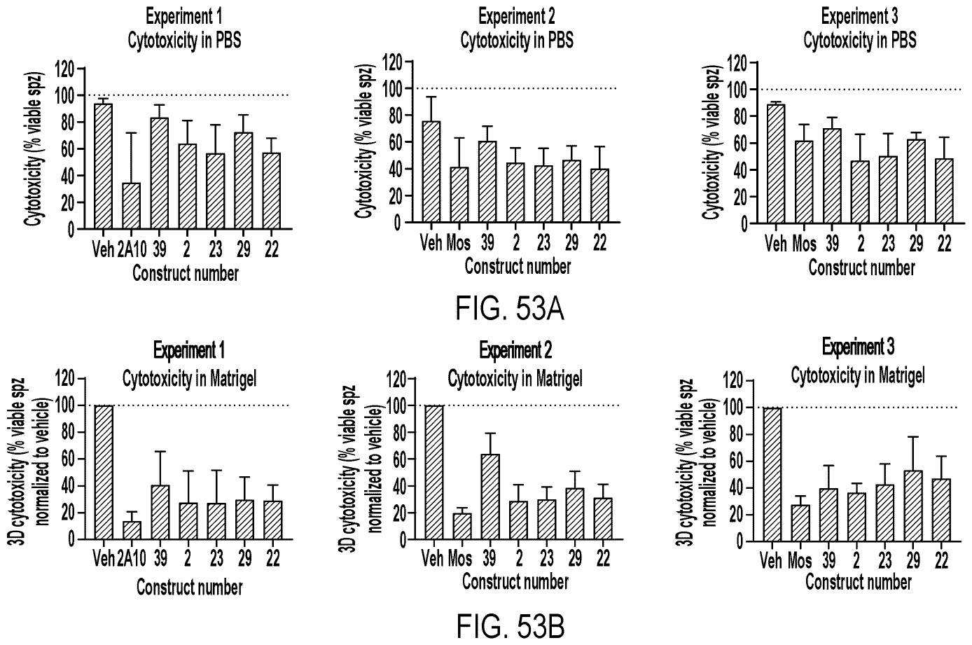

The cytotoxicity of serum from mice immunized with RNA constructs 2, 22, 23, 29, and 39 was evaluated both in suspension (in PBS) and in a 3D Matrigel matrix as described above across three experiments. As shown in FIG. 53A-53B, in Experiment 1, the highest impact in sporozoite viability in PBS was observed for RNA constructs 2, 23 and 22. In Experiments 2 and 3, the lowest viability was observed for sporozoites incubated with sera from mice immunized with RNA constructs 2, 23, 29 and 22, similarly to what was observed for the positive control, Mosquirix®. The decrease in viability of the sporozoites was more pronounced when the incubation was done in Matrigel than when it was done in PBS.

Thus, the present Example demonstrates that tested RNA constructs induced an immune response characterized by production of antibodies that are shown to differentially reduce viable sporozoites as assessed using a cytotoxicity assay. For example, in suspension, sera from mice immunized with most RNA constructs reduced viable sporozoites (exhibited by a mean of at least about 80% viable sporozoite) compared to vehicle, while sera from mice immunized with all tested RNA constructs reduced viable sporozoites in 3D (i.e., Matrigel) (exhibited by a mean of at least about 40% viable sporozoite).

### (12) In Vitro Gliding Assay

Provided polyribonucleotides can be assessed for their ability to induce production of antibodies with an inhibitory effect on sporozoite motility. In some embodiments, a polyribonucleotide is determined to induce a useful immune response if serum from a subject (e.g., a mouse) immunized with such construct is shown to reduce sporozoite gliding speed in an In vitro gliding assay, as described herein.

In the gliding assay, 5,000 PbPf sporozoites were resuspended in Dulbecco's modified Eagle medium (DMEM) and incubated with 5% serum (from mice immunized with either a positive control anti-PfCSP 2A10 mAb IV or RNA constructs (as described in Example 1). The resulting suspension was transferred to an 18-well slide and centrifuged at 400×g for 3 min at 4° C. The slide was then allowed to equilibrate at 37° C., 5% CO2 for 3 min in the incubation chamber (Incubation System S) of an inverted epifluorescence wide-field microscope (AxioObserver Z.1) equipped with an LED illumination system (Colibri2), a CCD camera (AxioCam MR) and controlled by the AxioVision software (version 4.8.2.0). Time-lapse movies were then recorded for 2 min at a rate of one image per second with an EC “Plan-Neofluar” 10×/0.3 objective using a 470 nm LED and a matching filter cube (43HE) to excite and detect GFP and thus visualize sporozoites. Average sporozoite velocity over the first 2 min of the acquisition was determined using the MTrack2 plug-in from Fiji.

As shown in FIG. 47E, sera from mice immunized with RNA construct 39 reduced sporozoite gliding speed (exhibited by a mean speed of at least about 1.1 μm/s) as compared to sera from mice administered a vehicle (exhibited by a mean speed of at least about 1.3 μm/s). Sera from mice immunized with anti-PfCSP 2A10 mAb IV displayed the greatest reduction in sporozoite gliding speed (exhibited by a mean speed of at least about 0.6 μm/s). Moreover, as shown in FIG. 49B, sporozoites incubated with serum from mice immunized with RNA constructs 31, 41, 2, 23, 28, 29 and 22 or those passively immunized with 2A₁₀, displayed a similar decrease in gliding speed in comparison with sporozoites incubated with serum from mice immunized with vehicle alone.

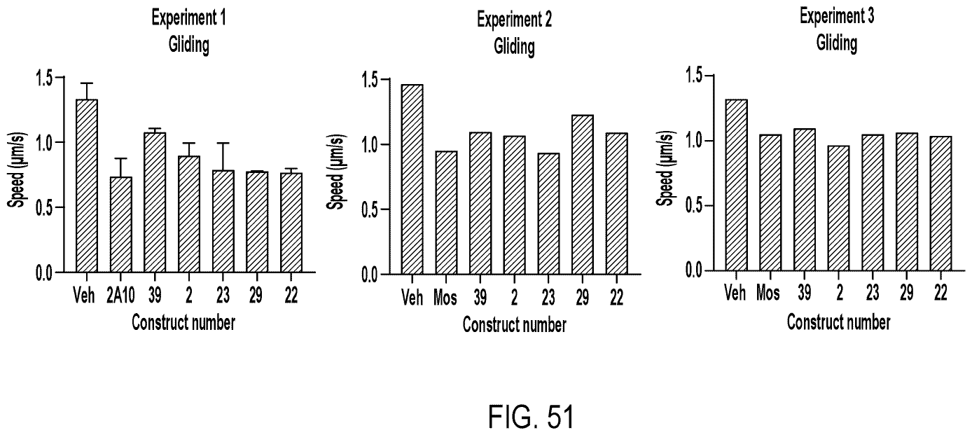

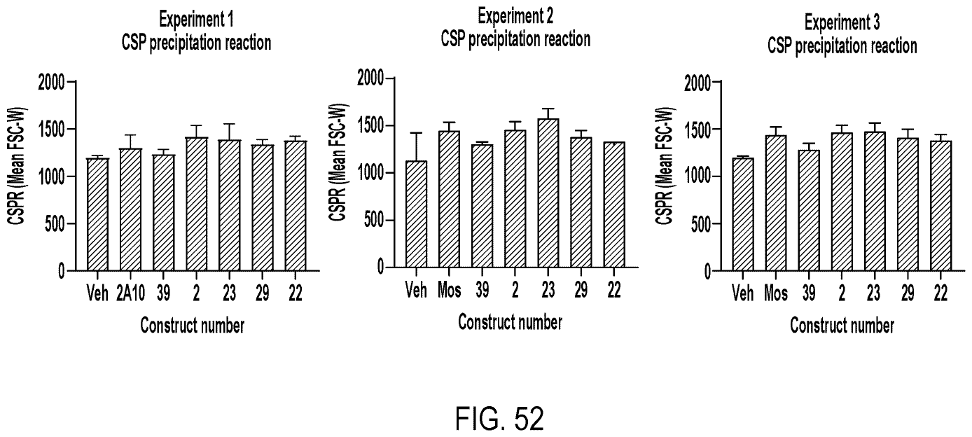

Three sporozoites gliding experiments were performed to assess RNA constructs 2, 22, 23, 29, and 39 specifically. As shown in FIG. 51, in all experiments, all of the RNA constructs showed an inhibition of the gliding motility of sporozoites in comparison to the vehicle groups. In Experiment 1, the strongest inhibition of gliding was observed for RNA constructs 23, 29 and 22, and to a similar extent as the inhibition observed for the 2A10 group. In Experiment 2, the strongest inhibition was observed for RNA construct 23, similarly to Mosquirix®. In Experiment 3, inhibition of gliding was very similar for all the tested constructs, and also for Mosquirix®.

Thus, the present Example demonstrates that most RNA constructs compared to vehicle induced an immune response characterized by production of antibodies that are shown to reduce motility of sporozoites, e.g., assessed using an in vitro gliding assay. For example, RNA constructs 41, 23, 28, 29 and 22 induced an immune response characterized by production of antibodies that are shown to reduce motility of sporozoites similar to the positive control.

### Example 3: Protection Studies of Exemplary Polyribonucleotides Encoding Plasmodium Polypeptide Constructs

The present Example documents the ability of certain polyribonucleotides, provided by the present disclosure, to induce immune responses, as assessed in mice.

Provided polyribonucleotides can be assessed for their ability to protect a subject from sporozoite challenge. In some embodiments, a provided polyribonucleotide is determined to induce a useful immune response if a subject (e.g., a mouse) immunized with a polyribonucleotide and injected with sporozoites demonstrate a reduced level of infection as assessed by monitoring blood parasitemia, e.g., in a challenge assay as described herein.

A challenge assay was performed in which C57BL/6 female mice (7-week-old, ˜20 g at day 0) were immunized intramuscularly (IM) twice, on days 0 and 21, with 1 μg of a RNA construct (as described in Example 1) or injected with vehicle (n=7 mice/group). Blood samples were collected on days 7, 14, 20, 28, 35, 42, and 49 for analysis of antibody titers and functionality in serum. Sporozoite challenge was performed at day 50 by micro-injection of 5000 Plasmodium falciparum CSP-expressing Plasmodium berghei ANKA GFP-fluorescent sporozoites freshly dissected from the salivary glands of infected female Anopheles mosquitoes. Protection against infection was assessed by monitoring blood parasitemia by flow cytometry until experimental day 60 (day 10 after challenge). The positive control group was passively immunized with 100 μg of anti-PfCSP 2A10 mAb IV, one day before infection.

Antibody titers were assessed by ELISA as described above in Example 2. Pre-challenge samples (day 49) are also used in functionality tests that include a fixed sporozoite assay, an inhibition of sporozoite motility assay, an assay to quantify cytotoxic activity of generated antibodies, and an assay that evaluated occurrence of CSP precipitation reaction as described above on Example 2 (data are shown in Example 2). Animals were divided into multiple treatment receiving groups, as exemplified in Table 15.

TABLE 15
General study plan for challenge studies with CSP constructs

| Size | | | | | D −7, 7, | | D 28, | | | D 50 to |
|---|---|---|---|---|---|---|---|---|---|---|
| (n) | Group | Treatment | Dose | D 0 | 14, 20 | D 21 | 35 | D 49 | D 50 | D 60 |
| 7 | 1 | Buffer | N/A | IM | Blood | IM | BC | BC | Infection | Blood |
| | | (NaCl) | | injection | collection | | | | with 5000 | parasitemia |
| 7 | 3-10 | F1-F8 | 1 | (20 μl | (BC) | | | | PICSP- | monitored |
| | | | ug | in one | | | | | expressing | by flow |
| | | | | leg) | | | | | P. berghei | cytometry |
| 7 | 2 | 2A10 | 100 | N/A | N/A | N/A | N/A | IV | spz | |
| | | | ug | | | | | injection | | |
| | | | | | | | | (150 μL) | | |

F1, F2, F3, . . . , F8—LNP formulations comprising RNA construct with modified nucleotides
2A10—mouse monoclonal antibody against PICSP major repeats
BC—Blood collection/Serum generation
Pre-immune serum collected at day −7 from all the mice
N/A—not applicable

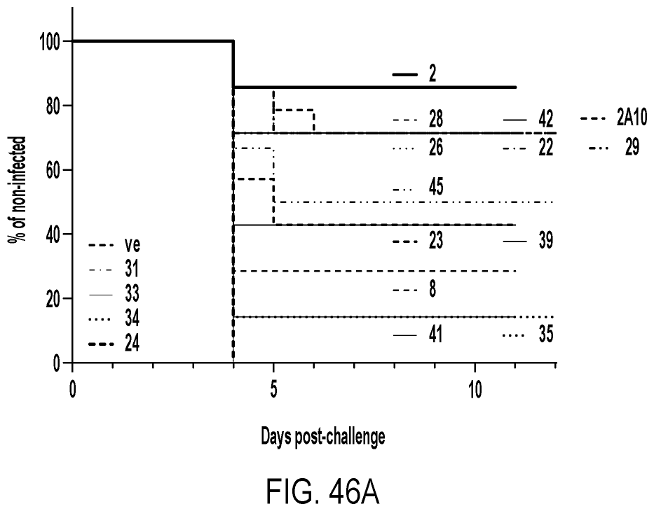

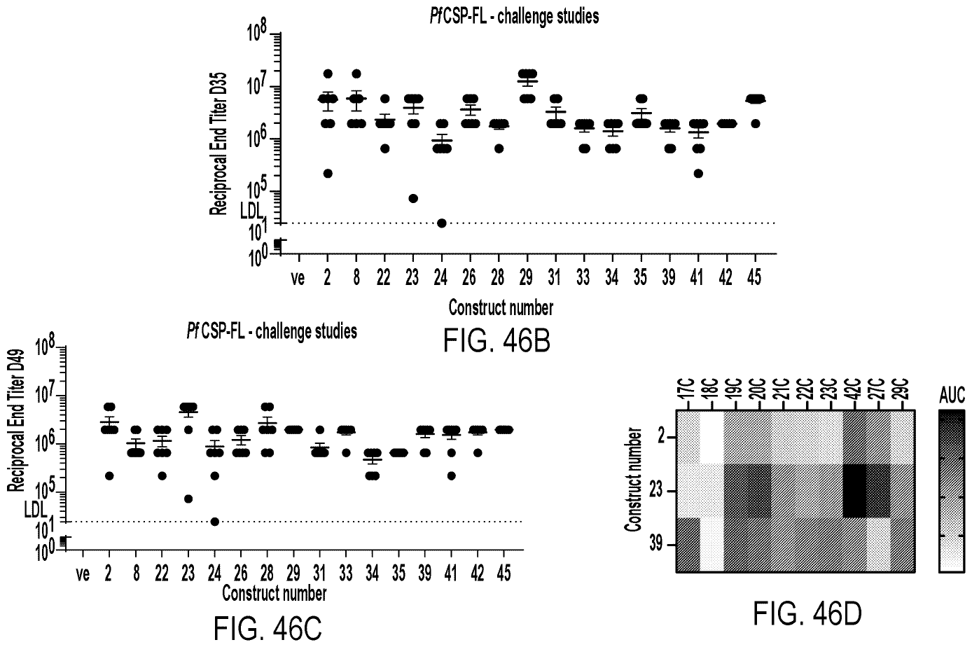

As shown in FIG. 46A, mice immunized with polyribonucleotide encoding a RNA construct 2 (as described in Example 1) were exhibited the highest level of protection after challenge with PfCSP-expressing P. berghei sporozoites with 6 out of 7 mice surviving at day 10. About 70% of mice immunized with polyribonucleotide encoding RNA constructs 26, 28, 42, a polyribonucleotide encoding a AN-term Plasmodium polypeptide construct (RNA construct 22) and a polyribonucleotide encoding a Plasmodium polypeptide construct that only includes major repeats and the C-term (RNA construct 29) were protected, which was comparable to protection following immunization with the positive control anti-PfCSP 2A10 mAb IV. Fewer than 50% of mice immunized with other tested constructs exhibited protection following challenge, and immunization with RNA constructs 31, 33, 34 and 24 provided no protection. As shown in FIGS. 46B and 46C, mice immunized with all tested RNA constructs showed high titers when measured in an ELISA assay against PfCSP full length protein at days 35 and 49, respectively. FIG. 46D shows binding between antibodies generated by immunization with RNA constructs 2, 23 and 39 during challenge studies, and specific PfCSP epitopes. Overall, full length RNA constructs 2 and 23 showed higher binding to epitopes 42C and 27C in the major repeat region.

Thus, the present Example demonstrates that certain polyribonucleotides effectively induced an immune response sufficient to inhibit and/or reduce level of infection by P. falciparum. For example, RNA construct 2 protected about 85% of mice following challenge, and RNA constructs 22, 26, 28, 29, and 42 protected about 70% of mice following challenge.

For selected constructs the challenge experiment was repeated twice (Experiment 2 and Experiment 3) as described above, except that the positive control was changed in these two experiments and mice were immunized twice IM with 5 μg of Mosquirix® as positive control.

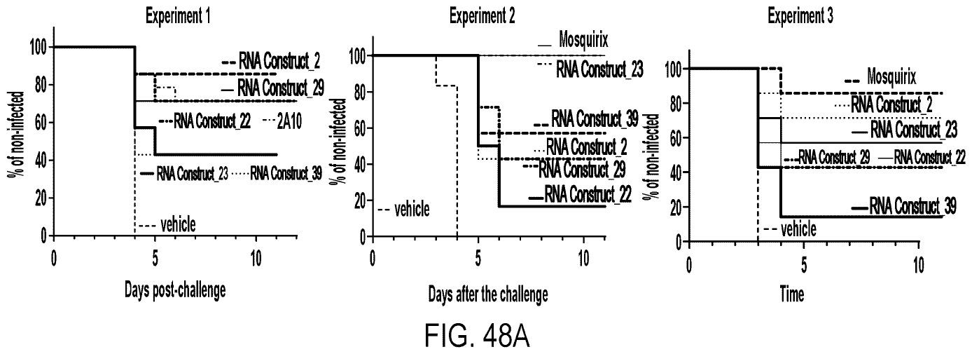

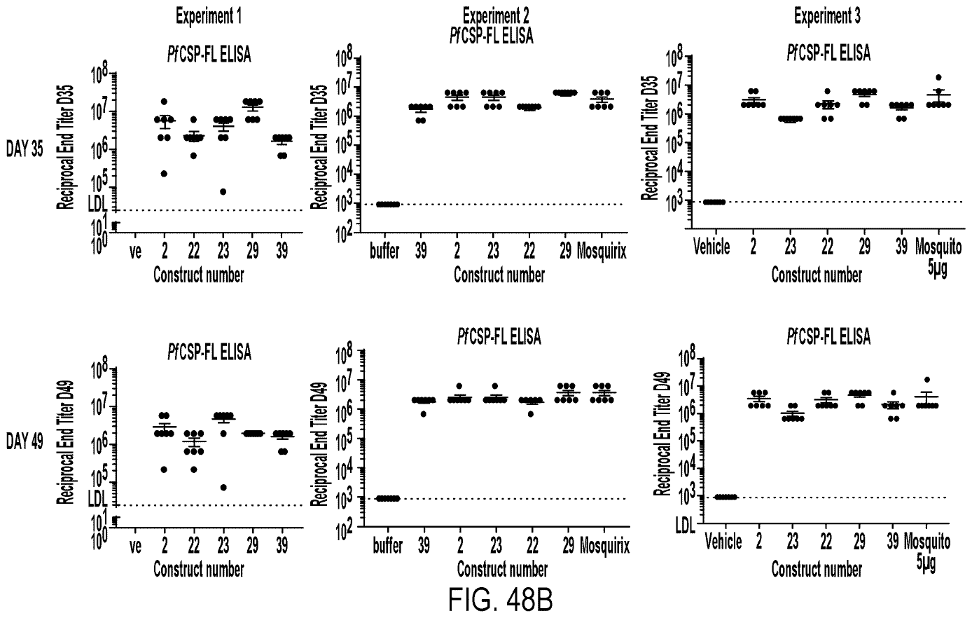

The results are shown in FIG. 48. As is evident from FIG. 48A, three challenge experiments were performed in total with RNA constructs 2, 22, 23, 29 and 39. Results from experiment 1 were described above. In experiment 2, all vehicle mice had blood stage parasitaemia by day 4 post sporozoite injection, whereas 1/7 mice immunized with RNA Construct 22 were protected, 3/7 mice immunized with RNA Construct 2 and RNA Construct 29 were protected, and 4/7 mice immunized with RNA Construct 39 were protected against infection. All mice from both the RNA Construct 23-immunized group and the Mosquirix® immunized group remained uninfected until day 11 post sporozoite challenge and were therefore fully protected. In experiment 3, blood stage infection was detected in all vehicle control mice already on day 3 post sporozoite injection. Six out of 7 mice immunized with Mosquirix® were fully protected, 5/7 mice immunized with RNA Construct 2 were protected, immunization with RNA Construct 23 protected 4/7 mice against infection, and mice immunized with RNA Construct 29, RNA Construct 22 and 39 protected less than 4 mice out of 7. As shown in FIG. 48B, mice immunized with RNA constructs 2, 22, 23, 29 and 39 showed high titers when measured in an ELISA assay against PfCSP full length protein at days 35 and 49, respectively.

### Example 4: In vivo Immunogenicity in Humans Following Administration of an Exemplary Polyribonucleotides Encoding Plasmodium CSP Polypeptide Construct

The present Example demonstrates that exemplary polyribonucleotides encoding different malarial polypeptide, as described herein, can be immunogenic in vivo in humans. Specifically, the present Example provides a randomized, dose-escalation trial for the evaluation of safety, tolerability, and immunogenicity of a formulated RNA construct as provided herein. The exemplary formulated RNA constructs to be assessed in this Example are RNA Construct 23 or RNA Construct 39.

### Trial Inclusion Criteria

Subjects to be included in a trial as described in this Example:
i. Are aged 18 to 55 years.
ii. Have a body mass index over 18.5 kg/m2 and under 35 kg/m2 and weigh at least 45 kg at an initial visit (“Visit 0”).
iii. Are healthy, in the clinical judgment of a health practitioner (e.g., a trial investigator) based on reported medical history data, physical examination, 12-lead electrocardiogram (ECG), vital signs, and clinical laboratory test outcomes. Healthy subjects with pre-existing stable disease (e.g., obesity, hypertension, etc.), defined as disease but not requiring significant change in therapy or hospitalization for worsening disease during the three months (e.g., 90 days) before Visit 0, can be included.
iv. Agree not to enroll in another trial with an investigational medicinal product (“IMP”) starting from Visit 0 and until 12 weeks after receiving a third dose of a formulated RNA construct or a combination of formulated RNA constructs.
v. Have not traveled and agree not to travel to a malaria-endemic region, as defined per Centers for Disease Control and Prevention (CDC) starting 6 months before Visit 0 and continuously until 28 days after receiving a third dose of a formulated RNA construct or a combination of formulated RNA constructs.
vi. Have a negative blood test result for human immunodeficiency virus (HIV) −1 and −2 at Visit 0.
vii. Have a negative blood test result for SARS-COV-2 at Visit 0.
viii. Have a negative blood test result for Hepatitis B surface antigen (HBsAg) at Visit 0 and negative anti Hepatitis C virus (anti-HCV) antibodies, or negative HCV PCR test result if the anti-HCV is positive at Visit 0.
ix. If a subject has childbearing potential, the subject has a negative serum beta-human chorionic gonadotropin (β-HCG) pregnancy test result at Visit 0 and negative urine pregnancy test results before each administration of a formulated RNA construct or a combination of formulated RNA constructs. Subjects born female who are postmenopausal or permanently sterilized are not be considered to have childbearing potential.
x. If a subject has childbearing potential, the subject agree to practice a highly effective form of contraception starting at Visit 0 and continuously until 90 days after receiving a third dose of a formulated RNA construct or a combination of formulated RNA constructs.
xi. If a subject has childbearing potential, the subject agree not to donate or cryopreserve eggs (ova, oocytes) for the purposes of assisted reproduction during trial, starting at Visit 0 and continuously until 90 days after receiving a third dose of a formulated RNA construct or a combination of formulated RNA constructs.
xii. If a subject is male, does not have had a vasectomy and are sexually active with partners of childbearing potential agree to use condoms and to practice a highly effective form of contraception with their sexual partners of childbearing potential during the trial, starting at Visit 0 and continuously until 90 days after receiving a third dose of a formulated RNA construct or a combination of formulated RNA constructs.
xiii. If a subject is male, will refrain from sperm donation, starting at Visit 0 and continuously until 90 days after receiving a third dose of a formulated RNA construct or a combination of formulated RNA constructs.

### Trial Exclusion Criteria

Subjects with the following are excluded from a trial described in the present Example:
i. A history of Plasmodium parasitemia (any species) based on subject-reported medical history.
ii. A prior residence for greater than 6 months continuously in a malaria-endemic region as defined per CDC at any point during their lifetime.
iii. Participation in breastfeeding or an intention to become pregnant or to father children starting with Visit 0 and continuously until 90 after receiving a third dose of a formulated RNA construct or a combination of formulated RNA constructs.
iv. A history of any serious adverse reactions to vaccines or to vaccine components such as lipids, and including history of anaphylaxis and related symptoms such as hives, respiratory difficulty, angioedema, and/or abdominal pain (not excluded from participation: a subject who had an anaphylactic adverse reaction to pertussis vaccine as a child).
v. Existence or history of the following medical conditions:
   a. Uncontrolled or moderate or severe respiratory diseases (e.g., asthma, chronic obstructive pulmonary disease); symptoms of asthma severity as defined in the US National Asthma Education and Prevention Program Expert Panel report, 2020—e.g., exclude a volunteer who:
      i. Uses a short-acting rescue inhaler (typically a beta 2 agonist) daily, or
      ii. Uses high dose inhaled corticosteroids (per American Academy of Allergy Asthma & Immunology), or
      iii. In the past year has either of the following:
         1. Greater than one exacerbation of symptoms treated with oral/parenteral corticosteroids; or
         2. Needed hospitalization, or intubation for asthma;
   b. Diabetes mellitus type 1 or type 2, including cases controlled with diet alone or elevated hemoglobin A₁C (HbAlc)≥6.5% at screening (not excluded: history of isolated gestational diabetes);
   c. Hypertension:
      i. If a person has a history of hypertension, or elevated blood pressure detected during screening, exclude for blood pressure that is not well controlled. Well controlled blood pressure is defined as consistently≤140 mm Hg systolic and ≤90 mm Hg diastolic, with or without medication, with only isolated, brief instances of higher readings, which must be ≤150 mm Hg systolic and ≤100 mm Hg diastolic at screening and enrollment;
   d. Malignancy within 5 years of screening, excluding localized basal or squamous cell skin cancer;
   e. Any current or history of cardiovascular diseases, (e.g., myocarditis, pericarditis, myocardial infarction, congestive heart failure, cardiomyopathy or clinically significant arrhythmias), unless such disease is not considered relevant for participation in this trial in a health practitioner's (e.g., investigator's) judgment;
   f. An abnormal screening ECG (i.e., showing the corrected QT interval by Fridericia (QTcF)>150 ms; significant ST-T wave changes suggestive of myocardial ischemia or of an acute or indeterminate-age myocardial infarction; left ventricular hypertrophy; any non-sinus rhythm including isolated premature ventricular contractions; complete right or left bundle branch block [QRS>120 ms]; second- or third-degree atrioventricular [AV] block); or other clinically significant abnormalities on the ECG at the investigator's discretion;
   g. Bleeding disorder diagnosed by a doctor (e.g., factor deficiency, coagulopathy, or platelet disorder requiring special precautions); or
   h. Seizure disorder: History of seizure(s) within past 3 years. Also exclude if volunteer has used medications in order to prevent or treat seizure(s) at any time within the past 3 years.
vi. Documented major psychiatric illness, including bipolar disorder, major depressive disorder, schizophrenia, autism, and attention deficit-hyperactivity disorder that at the discretion of the investigator could interfere with participation and follow-up as outlined by the trial.
vii. The following diseases associated with immune dysregulation:
   a. Primary immunodeficiencies;
   b. History of solid organ or bone marrow transplantation;
   C. Asplenia: any condition resulting in the absence of a functional spleen; or
   d. Existence or history of autoimmune disease including and not limited to thyroid autoimmune disease, multiple sclerosis, psoriasis, etc.
viii. Previous vaccination with an approved or investigational malaria vaccine at any time or having taken part in a human malaria challenge study.
ix. Receipt of any investigational product within 28 days before Visit 0.
x. Any planned non-trial vaccinations starting at Visit 0 and continuously until 28 days after a third dose of a formulated RNA construct or a combination of formulated RNA constructs. Seasonal influenza and COVID-19 vaccines are allowed; however, they should be administered at least 14 days before or 28 days after any IMP administration. Emergency vaccinations, such as tetanus, are allowed to be administered when medically indicated.
xi. Received blood/plasma products, monoclonal antibodies or immunoglobulin within 120 days before Visit 1 or planned administration starting at Visit 0 and continuously until Visit 21 (e.g., 365 days after administration of a third dose).
xii. Received allergy treatment with antigen injections within 28 days before and after each IMP administration.
xiii. Current or planned treatment with immunosuppressive therapy, including systemic corticosteroids (if systemic corticosteroids are administered for ≥14 days at a dose of ≥20 mg/d of prednisone or equivalent) starting at Visit 0 and continuously until a third dose of a formulated RNA construct or a combination of formulated RNA constructs. Intraarticular, intrabursal, or topical (skin or eyes) corticosteroids are permitted.
xiv. Have a history of alcohol abuse or drug addiction within 1 year before Visit 0 or have a history (within the past 5 years) of substance abuse, which in the opinion of a health practitioner (e.g., an investigator), could compromise their wellbeing if they participate as a subject in a trial, or that could prevent, limit, or confound the protocol specified assessments.
xv. Any existing condition which may affect vaccine injection and/or assessment of local reactions at the injection site, e.g., tattoos, severe scars, etc.
xvi. Are vulnerable individuals as per International Council for Harmonisation (ICH) E6 definition, i.e., are individuals whose willingness to volunteer in a clinical trial may be unduly influenced by the expectation, whether justified or not, of benefits associated with participation, or of a retaliatory response from senior members of a hierarchy in case of refusal to participate.
xvii. Any screening hematology and/or blood chemistry laboratory value that meets the definition of a Grade ≥2 abnormality or a Grade 1 abnormality at a health practitioner's (e.g., an investigator's) discretion at Visit 0. Subjects with abnormal but not clinically significant parameters not included in the toxicity guidance may be considered eligible at discretion of a health practitioner (e.g., an investigator).
xviii. Prior residence for greater than 6 months in a malaria endemic region.
xix. Sickle cell disease.

### Trial-RNA Construct 23

RNA Construct 23 is evaluated in different dose combinations in a 3 dose schedule to select a safe, tolerable, and immunogenic dose combination, and to assess the impact of third dose on immunogenicity.

To assess safety, tolerability, and immunogenicity of RNA Construct 23, subjects are divided into cohorts. Cohorts are randomized 5:1 active: placebo. Evaluation uses a staggered dose-escalation schema with sentinel subjects in all cohorts. Different cohorts receive different doses of RNA Construct 23. Table 16 provides an overview of the dose combinations used. A placebo cohort that receives isotonic NaCl solution (0.9%) is also assessed.

TABLE 16
Dose Combinations

| | Dose level (μg) RNA | Sample size |
|---|---|---|
| Cohort | Construct 23 | (active:placebo) |
| 1 | 10 | 10:2 |
| 2 | ≤30 | 10:2 |
| 3 | ≤70 | 10:2 |

A first dose of RNA Construct 23 is administered to subject on Day 1. Approximately eight weeks later, a second dose of RNA Construct 23 is administered to the subject. A third dose of RNA Construct 23 is administered approximately 18 weeks after the second dose.

### Trial-RNA Construct 39

RNA Construct 39 is evaluated in different dose combinations in a 3 dose schedule to select a safe, tolerable, and immunogenic dose combination, and to assess the impact of third dose on immunogenicity.

To assess safety, tolerability, and immunogenicity of RNA Construct 39, subjects are divided into cohorts. Cohorts are randomized 4:1 active: placebo. Evaluation uses a staggered dose-escalation schema with sentinel subjects in all cohorts. Cohort 1 receives a 3 μg dose of a formulated RNA Construct 39 construct, Cohort 2 receives a 10 μg or less dose of a formulated RNA Construct 39 construct, and Cohort 3 receives a 30 μg or less dose of a formulated RNA Construct 39 construct. Placebo cohorts receive isotonic NaCl solution (0.9%).

A first dose of RNA Construct 39 is administered to subject on Day 1. A second dose of RNA Construct 39 is administered to the subject approximately weeks later (e.g., around Day 57). Approximately 18 weeks after the second dose (e.g., around Day 183), a third dose of RNA Construct 39 is administered.

### Post-Trial Assessments

Subjects are assessed for the following primary outcome measures after each dose of a formulated RNA construct (e.g., RNA Construct 23 or RNA Construct 39):
i. Frequency of solicited local reactions at the injection site (e.g., pain, erythema/redness and/or induration/swelling) recorded up to 7 days after each dose;
ii. Frequency of solicited systemic reactions (vomiting, diarrhea, headache, fatigue, muscle/joint pain, and fever) recorded up to 7 days after each dose;
iii. Frequency of subjects with at least one AE occurring until 28 days after each dose; and
iv. Frequency of subjects with at least one medically attended adverse event (MAAE) occurring until 28 days after each dose.

Subjects are assessed to determine a frequency of subjects in each cohort with at least one SAE occurring until 24 weeks after a third dose.

Descriptive statistics on antibody levels (e.g., anti-CSP antibodies) in a subject can be assessed at various time points. For example, levels of antigen-specific serum and/or plasma antibodies can be assessed using ELISA or similar assays. Additionally, the functionality and/or avidity of serum and/or plasma antibodies are evaluated.

Other immune responses of subjects following administration of one or more doses are also assessed. For instance, CD4+ and CD8+ T-cell responses to antigens in a formulated RNA construct can be measured using polychromatic flow cytometry. Vaccine-induced plasmablasts and/or memory B cells can be measured using flow cytometry, and levels of inflammatory markers, chemokines, and/or cytokines can be measured.

RNA expression patterns in subjects following administration of one or more doses can be assessed using, e.g., RNASeq technology.

### EQUIVALENTS

Those skilled in the art will recognize, or be able to ascertain using no more than routine experimentation, many equivalents to the specific embodiments of technologies described herein. The scope of the present disclosure is not intended to be limited to the above Description, but rather is as set forth in the following claims.

1-48. (canceled)

49. An RNA construct comprising in 5′ to 3′ order:
(i) a 5′ UTR that comprises or consists of a modified human alpha-globin 5′-UTR;
(ii) a polyribonucleotide encoding a polypeptide that comprises the amino acid sequence of SEQ ID NO: 1 or an amino acid sequence having at least 98% or at least 99% sequence identity to an amino acid sequence according to SEQ ID NO: 1;
(iii) a 3′ UTR that comprises or consists of a first sequence from the amino terminal enhancer of split
(AES) messenger RNA and a second sequence from the mitochondrial encoded 125 ribosomal RNA; and
(iv) a polyA tail sequence.

50. The RNA construct of claim 49, further comprising a 5′ cap.

51. The RNA construct of claim 50, wherein the 5′ cap is a cap0, a cap1, or cap2.

52. The RNA construct of claim 51, wherein the 5′ cap is a cap1 structure comprising m7(3′0MeG) (5′) ppp (5′) (2′0MeA₁) PG₂, wherein A₁ is position +1 of the RNA construct, and G₂ is position +2 of the polyribonucleotide, and wherein the RNA construct further comprises the nucleic acid sequence A₃A₄U₅ (SEQ ID NO: 424) at positions +3, +4 and +5 respectively of the RNA construct.

53. The RNA construct of claim 49, wherein the 5′ UTR comprises the nucleic acid sequence according to SEQ ID NO: 415, or a nucleic acid sequence having at least 90%, at least 95%, or at least 97% identity to the nucleic acid sequence according to SEQ ID NO: 415.

54. The RNA construct of claim 49, wherein the 3′UTR comprises the nucleic acid sequence according to SEQ ID NO: 416, or a nucleotide sequence having at least 98%, or at least 99% identity to the nucleic acid sequence according to SEQ ID NO: 416.

55. The RNA construct of claim 49, wherein the polyA tail sequence comprises the nucleotide sequence according to SEQ ID NO: 428, or a nucleotide sequence having at least 96%, at least 97%, at least 98%, or at least 99% identity to the nucleotide sequence of according to SEQ ID NO: 428.

56. The RNA construct of claim 49, wherein the RNA construct comprises N₁-methylpseudouridine in place of each uridine.

57. The RNA construct of claim 49, which is codon-optimized.

58. A composition comprising one or more RNA constructs of claim 49.

59. The composition of claim 58, further comprising lipid nanoparticles, polyplexes (PLX), lapidated polyplexes (LPLX), or liposomes, wherein the one or more RNA constructs are fully or partially encapsulated within the lipid nanoparticles, polyplexes (PLX), lipidated polyplexes (LPLX), or liposomes.

60. A pharmaceutical composition comprising the composition of claim 58 and at least one pharmaceutically acceptable excipient.

61. A method of treating or preventing a malaria infection comprising administering to a subject an RNA construct according to claim 49.

62. A method of treating or preventing a malaria infection comprising administering to a subject a composition according to claim 58.

63. A method of treating or preventing a malaria infection comprising administering to a subject a pharmaceutical composition of claim 60.


SEQUENCE LISTING (450 sequences)

SEQ ID NO: 1 - protein, 397 aa, unidentified
  source 1..397: note = Plasmodium falciparum
MMRKLAILSVSSFLFVEALFQEYQCYGSSSNTRVLNELNYDNAGTNLYNELEMNYYGKQENWYSLKKNSRSLGENDDGNNEDNEKLRKPKHKKLKQPADGNPDPNANPNVDPNANPNVDPNANPNVDPNANPNANPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVFNVVNSSIGLIMVLSFLFLN

SEQ ID NO: 2 - DNA, 1134 bp, synthetic construct
  source 1..1134: mol_type = other DNA
atgatgagaaaattagctattttatctgtttcttcctttttatttgttgaggccttattccaggaataccagtgctatggaagttcgtcaaacacaagggttctaaatgaattaaattatgataatgcaggcactaatttatataatgaattagaaatgaattattatgggaaacaggaaaattggtatagtcttaaaaaaaatagtagatcacttggagaaaatgatgatggaaataacgaagacaacgagaaattaaggaaaccaaaacataaaaaattaaagcaaccagcggatggtaatcctgatccaaatgcaaacccaaatgtagatcccaatgccaacccaaatgtagatccaaatgcaaacccaaatgtagatccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaaccccaatgcaaatcctaatgcaaacccaaatgcaaacccaaacgtagatcctaatgcaaatccaaatgcaaacccaaacgcaaaccccaatgcaaatcctaatgcaaaccccaatgcaaatcctaatgcaaatcctaatgccaatccaaatgcaaatccaaatgcaaacccaaacgcaaaccccaatgcaaatcctaatgccaatccaaatgcaaatccaaatgcaaacccaaatgcaaacccaaatgcaaaccccaatgcaaatcctaataaaaacaatcaaggtaatggacaaggtcacaatatgccaaatgacccaaaccgaaatgtagatgaaaatgctaatgccaacagtgctgtaaaaaataataataacgaagaaccaagtgataagcacataaaagaatatttaaacaaaatacaaaattctctttcaactgaatggtccccatgtagtgtaacttgtggaaatggtattcaagttagaataaagcctggctctgctaataaacctaaagacgaattagattatgcaaatgatattgaaaaaaaaatttgtaaaatggaaaaatgttccagttgatga

SEQ ID NO: 3 - protein, 376 aa, synthetic construct
MMRKLAILSVSSFLFVEALFQEYQCYGSSSNTRVLNELNYDNAGTNLYNELEMNYYGKQENWYSLKKNSRSLGENDDGNNEDNEKLRKPKHKKLKQPADGNPDPNANPNVDPNANPNVDPNANPNVDPNANPNANPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSS

SEQ ID NO: 4 - RNA, 1134 nt, synthetic construct
  source 1..1134: mol_type = other RNA
atgatgagaaaattagctattttatctgtttcttcctttttatttgttgaggccttattccaggaataccagtgctatggaagttcgtcaaacacaagggttctaaatgaattaaattatgataatgcaggcactaatttatataatgaattagaaatgaattattatgggaaacaggaaaattggtatagtcttaaaaaaaatagtagatcacttggagaaaatgatgatggaaataacgaagacaacgagaaattaaggaaaccaaaacataaaaaattaaagcaaccagcggatggtaatcctgatccaaatgcaaacccaaatgtagatcccaatgccaacccaaatgtagatccaaatgcaaacccaaatgtagatccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaaccccaatgcaaatcctaatgcaaacccaaatgcaaacccaaacgtagatcctaatgcaaatccaaatgcaaacccaaacgcaaaccccaatgcaaatcctaatgcaaaccccaatgcaaatcctaatgcaaatcctaatgccaatccaaatgcaaatccaaatgcaaacccaaacgcaaaccccaatgcaaatcctaatgccaatccaaatgcaaatccaaatgcaaacccaaatgcaaacccaaatgcaaaccccaatgcaaatcctaataaaaacaatcaaggtaatggacaaggtcacaatatgccaaatgacccaaaccgaaatgtagatgaaaatgctaatgccaacagtgctgtaaaaaataataataacgaagaaccaagtgataagcacataaaagaatatttaaacaaaatacaaaattctctttcaactgaatggtccccatgtagtgtaacttgtggaaatggtattcaagttagaataaagcctggctctgctaataaacctaaagacgaattagattatgcaaatgatattgaaaaaaaaatttgtaaaatggaaaaatgttccagttgatga

SEQ ID NO: 5 - DNA, 1134 bp, synthetic construct
  source 1..1134: mol_type = other DNA
atgatgcggaagctggccatcctgagcgtgtccagcttcctgtttgtggaagccctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcagcccgccgacggcaatcccgatcctaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcctaacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagcagctgatga

SEQ ID NO: 6 - protein, 376 aa, synthetic construct; identical to SEQ ID NO: 3

SEQ ID NO: 7 - RNA, 1134 nt, synthetic construct
  source 1..1134: mol_type = other RNA
atgatgcggaagctggccatcctgagcgtgtccagcttcctgtttgtggaagccctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcagcccgccgacggcaatcccgatcctaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcctaacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagcagctgatga

SEQ ID NO: 8 - skipped

SEQ ID NO: 9 - protein, 376 aa, synthetic construct; identical to SEQ ID NO: 3

SEQ ID NO: 10 - skipped

SEQ ID NO: 11 - DNA, 1134 bp, synthetic construct
  source 1..1134: mol_type = other DNA
atgatgagaaaactggcaattctgtctgtgtcttcttttctgtttgtggaagctctgtttcaggaataccagtgctacggatcttcttctaatacaagagtgctgaatgaactgaattacgataatgctggaacaaatctctacaatgaactggaaatgaattactacggaaaacaggaaaattggtactctctgaagaaaaattctagatctctgggagaaaatgatgatggaaataatgaagataatgaaaaactgagaaaaccaaaacacaagaaactgaagcagccagctgatggaaatccagatccaaatgcaaatccaaatgtggacccaaatgcaaatccaaatgtggacccaaatgcaaatccaaatgtggacccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgtggacccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaataaaaacaatcaaggaaatggacagggacacaatatgccaaatgatccaaatagaaatgtggatgaaaatgcaaatgcaaactctgctgtgaaaaataataataatgaagaaccatctgataagcacatcaaagaatacctgaataaaatccagaattctctgtctacagaatggtctccttgctctgtgacatgcggaaatggaatccaggtgagaatcaaaccaggatctgcaaataaaccaaaagatgaactggattatgcaaatgatattgaaaagaaaatttgcaaaatggaaaaatgctccagttgatga

SEQ ID NO: 12 - protein, 376 aa, synthetic construct; identical to SEQ ID NO: 3

SEQ ID NO: 13 - RNA, 1134 nt, synthetic construct
  source 1..1134: mol_type = other RNA
atgatgagaaaactggcaattctgtctgtgtcttcttttctgtttgtggaagctctgtttcaggaataccagtgctacggatcttcttctaatacaagagtgctgaatgaactgaattacgataatgctggaacaaatctctacaatgaactggaaatgaattactacggaaaacaggaaaattggtactctctgaagaaaaattctagatctctgggagaaaatgatgatggaaataatgaagataatgaaaaactgagaaaaccaaaacacaagaaactgaagcagccagctgatggaaatccagatccaaatgcaaatccaaatgtggacccaaatgcaaatccaaatgtggacccaaatgcaaatccaaatgtggacccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgtggacccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaataaaaacaatcaaggaaatggacagggacacaatatgccaaatgatccaaatagaaatgtggatgaaaatgcaaatgcaaactctgctgtgaaaaataataataatgaagaaccatctgataagcacatcaaagaatacctgaataaaatccagaattctctgtctacagaatggtctccttgctctgtgacatgcggaaatggaatccaggtgagaatcaaaccaggatctgcaaataaaccaaaagatgaactggattatgcaaatgatattgaaaagaaaatttgcaaaatggaaaaatgctccagttgatga

SEQ ID NO: 14 - DNA, 537 bp, synthetic construct
  source 1..537: mol_type = other DNA
atgaatgcaaatccaaatgcaaacccaaacgcaaaccccaatgcaaatcctaatgcaaaccccaatgcaaatcctaatgcaaatcctaatgccaatccaaatgcaaatccaaatgcaaacccaaacgcaaaccccaatgcaaatcctaatgccaatccaaatgcaaatccaaatgcaaacccaaatgcaaacccaaatgcaaaccccaatgcaaatcctaataaaaacaatcaaggtaatggacaaggtcacaatatgccaaatgacccaaaccgaaatgtagatgaaaatgctaatgccaacagtgctgtaaaaaataataataacgaagaaccaagtgataagcacataaaagaatatttaaacaaaatacaaaattctctttcaactgaatggtccccatgtagtgtaacttgtggaaatggtattcaagttagaataaagcctggctctgctaataaacctaaagacgaattagattatgcaaatgatattgaaaaaaaaatttgtaaaatggaaaaatgttccagttgatga

SEQ ID NO: 15 - protein, 177 aa, synthetic construct
MNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSS

SEQ ID NO: 16 - RNA, 537 nt, synthetic construct
  source 1..537: mol_type = other RNA
atgaatgcaaatccaaatgcaaacccaaacgcaaaccccaatgcaaatcctaatgcaaaccccaatgcaaatcctaatgcaaatcctaatgccaatccaaatgcaaatccaaatgcaaacccaaacgcaaaccccaatgcaaatcctaatgccaatccaaatgcaaatccaaatgcaaacccaaatgcaaacccaaatgcaaaccccaatgcaaatcctaataaaaacaatcaaggtaatggacaaggtcacaatatgccaaatgacccaaaccgaaatgtagatgaaaatgctaatgccaacagtgctgtaaaaaataataataacgaagaaccaagtgataagcacataaaagaatatttaaacaaaatacaaaattctctttcaactgaatggtccccatgtagtgtaacttgtggaaatggtattcaagttagaataaagcctggctctgctaataaacctaaagacgaattagattatgcaaatgatattgaaaaaaaaatttgtaaaatggaaaaatgttccagttgatga

SEQ ID NO: 17 - DNA, 537 bp, synthetic construct
  source 1..537: mol_type = other DNA
atgaacgctaaccccaacgccaatcctaacgcaaaccctaatgcaaatccgaacgctaatccaaatgctaaccctaacgctaacccgaatgctaaccccaatgcaaatccaaatgccaatcctaatgccaatccaaacgcaaaccccaacgctaacccaaacgctaatccgaacgccaatccgaatgcaaatcccaatgctaacccgaacgcaaatcccaacaagaacaatcaaggcaacggccagggccacaacatgcccaacgatcccaacagaaacgtggacgagaacgccaacgcaaacagcgccgtgaagaacaacaacaacgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggacgagctggactacgccaacgacatcgagaagaaaatctgcaagatggaaaagtgcagcagctgatga

SEQ ID NO: 18 - protein, 177 aa, synthetic construct; identical to SEQ ID NO: 15

SEQ ID NO: 19 - RNA, 537 nt, synthetic construct
  source 1..537: mol_type = other RNA
atgaacgctaaccccaacgccaatcctaacgcaaaccctaatgcaaatccgaacgctaatccaaatgctaaccctaacgctaacccgaatgctaaccccaatgcaaatccaaatgccaatcctaatgccaatccaaacgcaaaccccaacgctaacccaaacgctaatccgaacgccaatccgaatgcaaatcccaatgctaacccgaacgcaaatcccaacaagaacaatcaaggcaacggccagggccacaacatgcccaacgatcccaacagaaacgtggacgagaacgccaacgcaaacagcgccgtgaagaacaacaacaacgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggacgagctggactacgccaacgacatcgagaagaaaatctgcaagatggaaaagtgcagcagctgatga

SEQ ID NO: 20 - DNA, 1272 bp, synthetic construct
  source 1..1272: mol_type = other DNA
atggggggggctgccgccaggttgggggccgtgattttgtttgtcgtcatagtgggcctccatggggtccgcagccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcagcccgccgacggcaatcccgatcctaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcctaacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagcagcggcctgatcgccggcgcggtgggcggcagtctcctggcagccctggtcatttgcggaattgtgtactggatgcgccgccacactcaaaaagccccaaagcgcatacgcctcccccacatccggtgatga

SEQ ID NO: 21 - protein, 422 aa, synthetic construct
MGGAAARLGAVILFVVIVGLHGVRSQEYQCYGSSSNTRVLNELNYDNAGTNLYNELEMNYYGKQENWYSLKKNSRSLGENDDGNNEDNEKLRKPKHKKLKQPADGNPDPNANPNVDPNANPNVDPNANPNVDPNANPNANPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSGLIAGAVGGSLLAALVICGIVYWMRRHTQKAPKRIRLPHIR

SEQ ID NO: 22 - RNA, 1272 nt, synthetic construct
  source 1..1272: mol_type = other RNA
atggggggggctgccgccaggttgggggccgtgattttgtttgtcgtcatagtgggcctccatggggtccgcagccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcagcccgccgacggcaatcccgatcctaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcctaacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagcagcggcctgatcgccggcgcggtgggcggcagtctcctggcagccctggtcatttgcggaattgtgtactggatgcgccgccacactcaaaaagccccaaagcgcatacgcctcccccacatccggtgatga

SEQ ID NO: 23 - DNA, 1170 bp, synthetic construct
  source 1..1170: mol_type = other DNA
atgggtgttacaggaatcttgcagctgcctagagatcgattcaagaggacatcattctttctgtgggtgattatcctgttccaaagaacattttcccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcagcccgccgacggcaatcccgatcctaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcctaacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagcagctgatga

SEQ ID NO: 24 - protein, 388 aa, synthetic construct
MGVTGILQLPRDRFKRTSFFLWVIILFQRTFSQEYQCYGSSSNTRVLNELNYDNAGTNLYNELEMNYYGKQENWYSLKKNSRSLGENDDGNNEDNEKLRKPKHKKLKQPADGNPDPNANPNVDPNANPNVDPNANPNVDPNANPNANPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSS

SEQ ID NO: 25 - RNA, 1170 nt, synthetic construct
  source 1..1170: mol_type = other RNA
atgggtgttacaggaatcttgcagctgcctagagatcgattcaagaggacatcattctttctgtgggtgattatcctgttccaaagaacattttcccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcagcccgccgacggcaatcccgatcctaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcctaacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagcagctgatga

SEQ ID NO: 26 - DNA, 1119 bp, synthetic construct
  source 1..1119: mol_type = other DNA
atgatgcggaagctggccatcctgagcgtgtccagcttcctgtttgtggaagccctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaaggccgacggcaatcccgatcctaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcctaacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagcagctgatga

SEQ ID NO: 27 - protein, 371 aa, synthetic construct
MMRKLAILSVSSFLFVEALFQEYQCYGSSSNTRVLNELNYDNAGTNLYNELEMNYYGKQENWYSLKKNSRSLGENDDGNNEDNEKLRKPKHKADGNPDPNANPNVDPNANPNVDPNANPNVDPNANPNANPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSS

SEQ ID NO: 28 - RNA, 1119 nt, synthetic construct
  source 1..1119: mol_type = other RNA
atgatgcggaagctggccatcctgagcgtgtccagcttcctgtttgtggaagccctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaaggccgacggcaatcccgatcctaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcctaacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagcagctgatga

SEQ ID NO: 29 - DNA, 1089 bp, synthetic construct
  source 1..1089: mol_type = other DNA
atgggcggagctgctgctagactgggagccgtgatcctgttcgtggttatcgtgggactgcatggcgtgcggggaaagctgaaacagcctgccgatggcaaccccgatcctaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcctaacaagaacaatcaaggcaacggccagggccacaacatgcccaacgatcccaacagaaacgtggacgagaacgccaacgccaacagcgccgtgaagaacaacaacaacgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggacgagctggactacgccaacgacatcgagaagaaaatctgcaagatggaaaagtgcagcagcgtgggaggatccggtggtggcggcagcggcggcggacttattgccggcgctgttggaggatctctgctggccgctctggtcatctgtggcatcgtgtactggatgcggcggcacacacagaaggcccctaagagaatcagactgccccacatcagatgatga

SEQ ID NO: 30 - protein, 361 aa, synthetic construct
MGGAAARLGAVILFVVIVGLHGVRGKLKQPADGNPDPNANPNVDPNANPNVDPNANPNVDPNANPNANPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVGGSGGGGSGGGLIAGAVGGSLLAALVICGIVYWMRRHTQKAPKRIRLPHIR

SEQ ID NO: 31 - RNA, 1089 nt, synthetic construct
  source 1..1089: mol_type = other RNA
atgggcggagctgctgctagactgggagccgtgatcctgttcgtggttatcgtgggactgcatggcgtgcggggaaagctgaaacagcctgccgatggcaaccccgatcctaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcctaacaagaacaatcaaggcaacggccagggccacaacatgcccaacgatcccaacagaaacgtggacgagaacgccaacgccaacagcgccgtgaagaacaacaacaacgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggacgagctggactacgccaacgacatcgagaagaaaatctgcaagatggaaaagtgcagcagcgtgggaggatccggtggtggcggcagcggcggcggacttattgccggcgctgttggaggatctctgctggccgctctggtcatctgtggcatcgtgtactggatgcggcggcacacacagaaggcccctaagagaatcagactgccccacatcagatgatga

SEQ ID NO: 32 - DNA, 1197 bp, synthetic construct
  source 1..1197: mol_type = other DNA
atgatgcggaagctggccatcctgagcgtgtccagcttcctgtttgtggaagccctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcagcccgccgacggcaatcccgatcctaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcctaacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagcagcgtgttcaacgtggtcaacagcagcatcggcctgattatggtgctgtccttcctgttcctgaactgatga

SEQ ID NO: 33 - protein, 397 aa, synthetic construct; identical to SEQ ID NO: 1

SEQ ID NO: 34 - RNA, 1197 nt, synthetic construct
  source 1..1197: mol_type = other RNA
atgatgcggaagctggccatcctgagcgtgtccagcttcctgtttgtggaagccctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcagcccgccgacggcaatcccgatcctaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcctaacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagcagcgtgttcaacgtggtcaacagcagcatcggcctgattatggtgctgtccttcctgttcctgaactgatga

SEQ ID NO: 35 - DNA, 1062 bp, synthetic construct
  source 1..1062: mol_type = other DNA
atgggcggagctgctgctagactgggagccgtgatcctgttcgtggttatcgtgggactgcacggcgtgcgcggagaggataatgagaagctgagaaagcccaagcacaagaagctgaagcagcccgccgacggcaaccccgatcctaacgctaatcccaacgtggaccccaacgccaatcctaatgtcgacccaaacgcaaacccaaacgtcgaccctaatgctaaccccaatgttgatcccaatgcaaacccgaatgtggaccctaacgcaaatccaaatgttgatccgaacgccaatccgaacgttgacccgaatgctaatccaaacgtggacccaaatgcaaaccccaacgtggacccaaacaagaacaatcaaggcaacggccagggccacaacatgcccaacgatcccaacagaaatgtggacgagaatgccaatgccaacagcgccgtgaagaacaacaacaacgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggacgagctggactacgccaacgacatcgagaagaaaatctgcaagatggaaaagtgcagcagcgtcggcggatctggtggcggaggatctggcggagaactggacggctctaagaatgagtggggctggtccaagtctaagctgggcgctaatgctggcggcggaagcgaactggatggcagcaaaaacgaatggggatggtctaagagcaagctgggagccaacgcaggcggaggctctgaactcgatggctccaaaaatgagtggggatggtcaaaatccaagctcggggcaaatgccggtggcggctcagagctggacggaagcaagaatgaatggggctggtcaaaaagcaaactcggcgcaaacgcaggcggtggcagcgagcttgacggatcaaagaacgagtggggatggtccaaaagtaaactgggcgccaatgcctgatga

SEQ ID NO: 36 - protein, 352 aa, synthetic construct
MGGAAARLGAVILFVVIVGLHGVRGEDNEKLRKPKHKKLKQPADGNPDPNANPNVDPNANPNVDPNANPNVDPNANPNVDPNANPNVDPNANPNVDPNANPNVDPNANPNVDPNANPNVDPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVGGSGGGGSGGELDGSKNEWGWSKSKLGANAGGGSELDGSKNEWGWSKSKLGANAGGGSELDGSKNEWGWSKSKLGANAGGGSELDGSKNEWGWSKSKLGANAGGGSELDGSKNEWGWSKSKLGANA

SEQ ID NO: 37 - RNA, 1062 nt, synthetic construct
  source 1..1062: mol_type = other RNA
atgggcggagctgctgctagactgggagccgtgatcctgttcgtggttatcgtgggactgcacggcgtgcgcggagaggataatgagaagctgagaaagcccaagcacaagaagctgaagcagcccgccgacggcaaccccgatcctaacgctaatcccaacgtggaccccaacgccaatcctaatgtcgacccaaacgcaaacccaaacgtcgaccctaatgctaaccccaatgttgatcccaatgcaaacccgaatgtggaccctaacgcaaatccaaatgttgatccgaacgccaatccgaacgttgacccgaatgctaatccaaacgtggacccaaatgcaaaccccaacgtggacccaaacaagaacaatcaaggcaacggccagggccacaacatgcccaacgatcccaacagaaatgtggacgagaatgccaatgccaacagcgccgtgaagaacaacaacaacgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggacgagctggactacgccaacgacatcgagaagaaaatctgcaagatggaaaagtgcagcagcgtcggcggatctggtggcggaggatctggcggagaactggacggctctaagaatgagtggggctggtccaagtctaagctgggcgctaatgctggcggcggaagcgaactggatggcagcaaaaacgaatggggatggtctaagagcaagctgggagccaacgcaggcggaggctctgaactcgatggctccaaaaatgagtggggatggtcaaaatccaagctcggggcaaatgccggtggcggctcagagctggacggaagcaagaatgaatggggctggtcaaaaagcaaactcggcgcaaacgcaggcggtggcagcgagcttgacggatcaaagaacgagtggggatggtccaaaagtaaactgggcgccaatgcctgatga

SEQ ID NO: 38 - DNA, 1437 bp, synthetic construct
  source 1..1437: mol_type = other DNA
atgggcggagctgctgctagactgggagccgtgatcctgttcgtggttatcgtgggactgcatggcgtgcggggcgagaatgtgcaagtgtccgacgagctgttcaacgagctgctgaacagcgtggacgtgaacggcgaagtgaaagagaacatcctggaagaaagccaagtgaacgacgacatcttcaacagcctggtcaagagcgtgcagcaagagcagcagcacaacgtggaagaaaaggtggaagaatccgtcgaggaaaacgacgaggaaagcgttgaagaaaacgttgaggaaaatgtcgaagaaaacgatgacgagagcgtggccagcagcgtcgaagaatctatcgccagctccgtggacgagagcatcgacagcagcattgaggaaaacgtggcccctacagtggaagaaatcgtggccccaactgtcgaggaaattgtggctcccagcgtggtggaatctgtggcccctagcgttgaggaatcagttgaggaaaatgttgaggaaagcgtggccgagaacgtggaagaatctgtggccgagaatgtcgaggaatccgtcgctgaaaacgtcgaagaatccgttgcagagaatgtggaagaaagcgtcgcagaaaatgtcgaggaaagtgtcgccgctggcaacagagtgcggagatctgtgggcgaagataacgagaagctgcggaagcccaagcacaagaagctgaagcagcccgccgacggcaaccccgatcctaacgctaatcccaacgtggaccccaacgccaatcctaatgtcgacccaaacgcaaacccaaacgtcgaccctaatgctaaccccaatgttgatcccaatgcaaacccgaatgtggaccctaacgcaaatccaaatgttgatccgaacgccaatccgaacgttgacccgaatgctaatccaaacgtggacccaaatgcaaaccccaacgtggacccaaacaagaacaatcaaggcaacggccagggccacaacatgcccaacgatcccaacagaaatgtggatgagaacgccaacgctaacagcgccgtgaagaacaacaacaacgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggacgagctggactacgccaacgacatcgagaagaaaatctgcaagatggaaaagtgcagcagcgtgggaggatccggtggtggcggcagcggcggcggacttattgccggcgctgttggaggatctctgctggccgctctggtcatctgtggcatcgtgtactggatgcggcggcacacacagaaggcccctaagagaatcagactgccccacatcagatgatga

SEQ ID NO: 39 - protein, 477 aa, synthetic construct
MGGAAARLGAVILFVVIVGLHGVRGENVQVSDELFNELLNSVDVNGEVKENILEESQVNDDIFNSLVKSVQQEQQHNVEEKVEESVEENDEESVEENVEENVEENDDESVASSVEESIASSVDESIDSSIEENVAPTVEEIVAPTVEEIVAPSVVESVAPSVEESVEENVEESVAENVEESVAENVEESVAENVEESVAENVEESVAENVEESVAAGNRVRRSVGEDNEKLRKPKHKKLKQPADGNPDPNANPNVDPNANPNVDPNANPNVDPNANPNVDPNANPNVDPNANPNVDPNANPNVDPNANPNVDPNANPNVDPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVGGSGGGGSGGGLIAGAVGGSLLAALVICGIVYWMRRHTQKAPKRIRLPHIR

SEQ ID NO: 40 - RNA, 1437 nt, synthetic construct
  source 1..1437: mol_type = other RNA
atgggcggagctgctgctagactgggagccgtgatcctgttcgtggttatcgtgggactgcatggcgtgcggggcgagaatgtgcaagtgtccgacgagctgttcaacgagctgctgaacagcgtggacgtgaacggcgaagtgaaagagaacatcctggaagaaagccaagtgaacgacgacatcttcaacagcctggtcaagagcgtgcagcaagagcagcagcacaacgtggaagaaaaggtggaagaatccgtcgaggaaaacgacgaggaaagcgttgaagaaaacgttgaggaaaatgtcgaagaaaacgatgacgagagcgtggccagcagcgtcgaagaatctatcgccagctccgtggacgagagcatcgacagcagcattgaggaaaacgtggcccctacagtggaagaaatcgtggccccaactgtcgaggaaattgtggctcccagcgtggtggaatctgtggcccctagcgttgaggaatcagttgaggaaaatgttgaggaaagcgtggccgagaacgtggaagaatctgtggccgagaatgtcgaggaatccgtcgctgaaaacgtcgaagaatccgttgcagagaatgtggaagaaagcgtcgcagaaaatgtcgaggaaagtgtcgccgctggcaacagagtgcggagatctgtgggcgaagataacgagaagctgcggaagcccaagcacaagaagctgaagcagcccgccgacggcaaccccgatcctaacgctaatcccaacgtggaccccaacgccaatcctaatgtcgacccaaacgcaaacccaaacgtcgaccctaatgctaaccccaatgttgatcccaatgcaaacccgaatgtggaccctaacgcaaatccaaatgttgatccgaacgccaatccgaacgttgacccgaatgctaatccaaacgtggacccaaatgcaaaccccaacgtggacccaaacaagaacaatcaaggcaacggccagggccacaacatgcccaacgatcccaacagaaatgtggatgagaacgccaacgctaacagcgccgtgaagaacaacaacaacgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggacgagctggactacgccaacgacatcgagaagaaaatctgcaagatggaaaagtgcagcagcgtgggaggatccggtggtggcggcagcggcggcggacttattgccggcgctgttggaggatctctgctggccgctctggtcatctgtggcatcgtgtactggatgcggcggcacacacagaaggcccctaagagaatcagactgccccacatcagatgatga

SEQ ID NO: 41 - DNA, 1296 bp, synthetic construct
  source 1..1296: mol_type = other DNA
atgggcggagctgctgctagactgggagccgtgatcctgttcgtggttatcgtgggactgcacggcgtgcggggacaagagtatcagtgttacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcagcccgccgacggcaatcccgatcctaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcctaacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagcagcgtgggctctcctggctctggaagcggctctggctatatccctgaggctcctagagatggccaggcctacgtcagaaaggatggcgaatgggtgctgctgagcaccttcctgggcagatctctggaagtgctgtttcaaggccctggctgatga

SEQ ID NO: 42 - protein, 430 aa, synthetic construct
MGGAAARLGAVILFVVIVGLHGVRGQEYQCYGSSSNTRVLNELNYDNAGTNLYNELEMNYYGKQENWYSLKKNSRSLGENDDGNNEDNEKLRKPKHKKLKQPADGNPDPNANPNVDPNANPNVDPNANPNVDPNANPNANPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVGSPGSGSGSGYIPEAPRDGQAYVRKDGEWVLLSTFLGRSLEVLFQGPG

SEQ ID NO: 43 - RNA, 1296 nt, synthetic construct
  source 1..1296: mol_type = other RNA
atgggcggagctgctgctagactgggagccgtgatcctgttcgtggttatcgtgggactgcacggcgtgcggggacaagagtatcagtgttacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcagcccgccgacggcaatcccgatcctaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcctaacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagcagcgtgggctctcctggctctggaagcggctctggctatatccctgaggctcctagagatggccaggcctacgtcagaaaggatggcgaatgggtgctgctgagcaccttcctgggcagatctctggaagtgctgtttcaaggccctggctgatga

SEQ ID NO: 44 - DNA, 1815 bp, synthetic construct
  source 1..1815: mol_type = other DNA
atgggcggagctgctgctagactgggagccgtgatcctgttcgtggttatcgtgggactgcacggcgtgcgcggagaggataatgagaagctgagaaagcccaagcacaagaagctgaagcagcccgccgacggcaaccccgatcctaacgctaatcccaacgtggaccccaacgccaatcctaatgtcgacccaaacgcaaacccaaacgtcgaccctaatgctaaccccaatgttgatcccaatgcaaacccgaatgtggaccctaacgcaaatccaaatgttgatccgaacgccaatccgaacgttgacccgaatgctaatccaaacgtggacccaaatgcaaaccccaacgtggacccaaacaagaacaatcaaggcaacggccagggccacaacatgcccaacgatcccaacagaaatgtggacgagaatgccaatgccaacagcgccgtgaagaacaacaacaacgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggacgagctggactacgccaacgacatcgagaagaaaatctgcaagatggaaaagtgcagcagcgtggccggcaacagagtgcgaagatctgtgggagaagaagcccctaagcctgagaaagagatctgcggcctgaaagtgggcagactgctggactctgtgaaaggctggctgagcgtgtcccagcaagagaagtgccctctgaacaagtactgcgagaacaagattcaggccgaccagtacaacctggtgcctctgacctgtatccgttggcggagcctgaatcctgcctctccaacaggatctctcggcggcaaggatgtggtgtccaagatcgatgccgccatgagcaacttcaagaccctgttcgagcccatgaaggccgacctggccaagctggaagaagaagtgaagcggcaggtcctggacgcctggaaagctctggaacccctccagaaagaggtgtacagaagcacactggccagcggcagaatcgagagagccgtgttctacagcttcatggaaatgggcgacaacgtgaagctggacaactacttccagcctgccaatgtggaagaactgctgaagtacgcctgggctctgcccatgcacaaaaagcagcggtctatgtacgacctgatcggccagctggtgcagagcagcaagagccctatgctccagacactgcatgccgtggaactggccaccgtggtcaatcccgagctggaaaacagagagaacctgctgaacgaccaggtggtgcagctgcgggacaacctgtacaagaacagcttcgccacactggtgtctatcgccagacacttccccgaccacttcgacaccctgcggcagagactgttcaagctgcctgatggctctaagcctggcgccgacacactgcccaacatcgtgaactttatcgcccagctgcctagcgacgagctgaggctgagttctgtggatctgctgctccagagcctgaccgccgagaatggaaccctggtgcaggaccctgagtacgtgtacagactgtctcagctggcccacgccatgccttctctggtggatgtgaaggcccatcctgacctccagcagagcgtggacgatctgatggccaagttcaacacccctatcgacggcaagaccctccagtacttccagaacatcggcatcagccccagcagctctgtggctacatgatga

SEQ ID NO: 45 - protein, 603 aa, synthetic construct
MGGAAARLGAVILFVVIVGLHGVRGEDNEKLRKPKHKKLKQPADGNPDPNANPNVDPNANPNVDPNANPNVDPNANPNVDPNANPNVDPNANPNVDPNANPNVDPNANPNVDPNANPNVDPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVAGNRVRRSVGEEAPKPEKEICGLKVGRLLDSVKGWLSVSQQEKCPLNKYCENKIQADQYNLVPLTCIRWRSLNPASPTGSLGGKDVVSKIDAAMSNFKTLFEPMKADLAKLEEEVKRQVLDAWKALEPLQKEVYRSTLASGRIERAVFYSFMEMGDNVKLDNYFQPANVEELLKYAWALPMHKKQRSMYDLIGQLVQSSKSPMLQTLHAVELATVVNPELENRENLLNDQVVQLRDNLYKNSFATLVSIARHFPDHFDTLRQRLFKLPDGSKPGADTLPNIVNFIAQLPSDELRLSSVDLLLQSLTAENGTLVQDPEYVYRLSQLAHAMPSLVDVKAHPDLQQSVDDLMAKFNTPIDGKTLQYFQNIGISPSSSVAT

SEQ ID NO: 46 - RNA, 1815 nt, synthetic construct
  source 1..1815: mol_type = other RNA
atgggcggagctgctgctagactgggagccgtgatcctgttcgtggttatcgtgggactgcacggcgtgcgcggagaggataatgagaagctgagaaagcccaagcacaagaagctgaagcagcccgccgacggcaaccccgatcctaacgctaatcccaacgtggaccccaacgccaatcctaatgtcgacccaaacgcaaacccaaacgtcgaccctaatgctaaccccaatgttgatcccaatgcaaacccgaatgtggaccctaacgcaaatccaaatgttgatccgaacgccaatccgaacgttgacccgaatgctaatccaaacgtggacccaaatgcaaaccccaacgtggacccaaacaagaacaatcaaggcaacggccagggccacaacatgcccaacgatcccaacagaaatgtggacgagaatgccaatgccaacagcgccgtgaagaacaacaacaacgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggacgagctggactacgccaacgacatcgagaagaaaatctgcaagatggaaaagtgcagcagcgtggccggcaacagagtgcgaagatctgtgggagaagaagcccctaagcctgagaaagagatctgcggcctgaaagtgggcagactgctggactctgtgaaaggctggctgagcgtgtcccagcaagagaagtgccctctgaacaagtactgcgagaacaagattcaggccgaccagtacaacctggtgcctctgacctgtatccgttggcggagcctgaatcctgcctctccaacaggatctctcggcggcaaggatgtggtgtccaagatcgatgccgccatgagcaacttcaagaccctgttcgagcccatgaaggccgacctggccaagctggaagaagaagtgaagcggcaggtcctggacgcctggaaagctctggaacccctccagaaagaggtgtacagaagcacactggccagcggcagaatcgagagagccgtgttctacagcttcatggaaatgggcgacaacgtgaagctggacaactacttccagcctgccaatgtggaagaactgctgaagtacgcctgggctctgcccatgcacaaaaagcagcggtctatgtacgacctgatcggccagctggtgcagagcagcaagagccctatgctccagacactgcatgccgtggaactggccaccgtggtcaatcccgagctggaaaacagagagaacctgctgaacgaccaggtggtgcagctgcgggacaacctgtacaagaacagcttcgccacactggtgtctatcgccagacacttccccgaccacttcgacaccctgcggcagagactgttcaagctgcctgatggctctaagcctggcgccgacacactgcccaacatcgtgaactttatcgcccagctgcctagcgacgagctgaggctgagttctgtggatctgctgctccagagcctgaccgccgagaatggaaccctggtgcaggaccctgagtacgtgtacagactgtctcagctggcccacgccatgccttctctggtggatgtgaaggcccatcctgacctccagcagagcgtggacgatctgatggccaagttcaacacccctatcgacggcaagaccctccagtacttccagaacatcggcatcagccccagcagctctgtggctacatgatga

SEQ ID NO: 47 - DNA, 1290 bp, synthetic construct
  source 1..1290: mol_type = other DNA
atgatgcggaagctggccatcctgagcgtgtccagcttcctgtttgtggaagccctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcagcccgccgacggcaatcccgatcctaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcctaacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagcagcggcggaggcggatctggtggcggaggaagtggtggcggcggatctcctaacaaaggcagcggcacaacctccggcaccacaagactgctgtctggccacacctgtttcaccctgactggcctgctgggcaccctggttacaatgggactgctgacatgatga

SEQ ID NO: 48 - protein, 428 aa, synthetic construct
MMRKLAILSVSSFLFVEALFQEYQCYGSSSNTRVLNELNYDNAGTNLYNELEMNYYGKQENWYSLKKNSRSLGENDDGNNEDNEKLRKPKHKKLKQPADGNPDPNANPNVDPNANPNVDPNANPNVDPNANPNANPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSGGGGSGGGGSGGGGSPNKGSGTTSGTTRLLSGHTCFTLTGLLGTLVTMGLLT

SEQ ID NO: 49 - RNA, 1290 nt, synthetic construct
  source 1..1290: mol_type = other RNA
atgatgcggaagctggccatcctgagcgtgtccagcttcctgtttgtggaagccctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcagcccgccgacggcaatcccgatcctaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcctaacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagcagcggcggaggcggatctggtggcggaggaagtggtggcggcggatctcctaacaaaggcagcggcacaacctccggcaccacaagactgctgtctggccacacctgtttcaccctgactggcctgctgggcaccctggttacaatgggactgctgacatgatga

SEQ ID NO: 50 - DNA, 618 bp, synthetic construct
  source 1..618: mol_type = other DNA
atgggcggagctgctgctagactgggagccgtgatcctgttcgtggttatcgtgggactgcatggcgtgcggggcaagtacaacgccaatcctaacgctaaccccaacgcaaaccctaatgccaatccaaatgcaaatcccaacgctaatccgaatgctaacccaaatgctaacccgaacgcaaatccaaacgcaaacccaaacgccaatccgaacgctaatcctaatgccaatcctaacgccaatcctaatgcaaacccgaatgctaaccccaatgccaatcctaatgctaaccctaacaagaacaatcaaggcaacggccagggccacaacatgcccaacgatcccaacagaaacgtggacgagaacgccaacgccaacagcgccgtgaagaacaacaacaacgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggacgagctggactacgccaacgacatcgagaagaaaatctgcaagatggaaaagtgcagcagcgtgtgatga

SEQ ID NO: 51 - protein, 204 aa, synthetic construct
MGGAAARLGAVILFVVIVGLHGVRGKYNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSV

SEQ ID NO: 52 - RNA, 618 nt, synthetic construct
  source 1..618: mol_type = other RNA
atgggcggagctgctgctagactgggagccgtgatcctgttcgtggttatcgtgggactgcatggcgtgcggggcaagtacaacgccaatcctaacgctaaccccaacgcaaaccctaatgccaatccaaatgcaaatcccaacgctaatccgaatgctaacccaaatgctaacccgaacgcaaatccaaacgcaaacccaaacgccaatccgaacgctaatcctaatgccaatcctaacgccaatcctaatgcaaacccgaatgctaaccccaatgccaatcctaatgctaaccctaacaagaacaatcaaggcaacggccagggccacaacatgcccaacgatcccaacagaaacgtggacgagaacgccaacgccaacagcgccgtgaagaacaacaacaacgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggacgagctggactacgccaacgacatcgagaagaaaatctgcaagatggaaaagtgcagcagcgtgtgatga

SEQ ID NO: 53 - DNA, 771 bp, synthetic construct
  source 1..771: mol_type = other DNA
atgggcggagctgctgctagactgggagccgtgatcctgttcgtggttatcgtgggactgcatggcgtgcggggcaagtacaacgccaatcctaacgctaaccccaacgcaaaccctaatgccaatccaaatgcaaatcccaacgctaatccgaatgctaacccaaatgctaacccgaacgcaaatccaaacgcaaacccaaacgccaatccgaacgctaatcctaatgccaatcctaacgccaatcctaatgcaaacccgaatgctaaccccaatgccaatcctaatgctaaccctaacaagaacaatcaaggcaacggccagggccacaacatgcccaacgatcccaacagaaacgtggacgagaacgccaacgccaacagcgccgtgaagaacaacaacaacgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggacgagctggactacgccaacgacatcgagaagaaaatctgcaagatggaaaagtgcagcagcgtgggaggatccggtggtggcggcagcggcggcggacttattgccggcgctgttggaggatctctgctggccgctctggtcatctgtggcatcgtgtactggatgcggcggcacacacagaaggcccctaagagaatcagactgccccacatcagatgatga

SEQ ID NO: 54 - protein, 255 aa, synthetic construct
MGGAAARLGAVILFVVIVGLHGVRGKYNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVGGSGGGGSGGGLIAGAVGGSLLAALVICGIVYWMRRHTQKAPKRIRLPHIR

SEQ ID NO: 55 - RNA, 771 nt, synthetic construct
  source 1..771: mol_type = other RNA
atgggcggagctgctgctagactgggagccgtgatcctgttcgtggttatcgtgggactgcatggcgtgcggggcaagtacaacgccaatcctaacgctaaccccaacgcaaaccctaatgccaatccaaatgcaaatcccaacgctaatccgaatgctaacccaaatgctaacccgaacgcaaatccaaacgcaaacccaaacgccaatccgaacgctaatcctaatgccaatcctaacgccaatcctaatgcaaacccgaatgctaaccccaatgccaatcctaatgctaaccctaacaagaacaatcaaggcaacggccagggccacaacatgcccaacgatcccaacagaaacgtggacgagaacgccaacgccaacagcgccgtgaagaacaacaacaacgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggacgagctggactacgccaacgacatcgagaagaaaatctgcaagatggaaaagtgcagcagcgtgggaggatccggtggtggcggcagcggcggcggacttattgccggcgctgttggaggatctctgctggccgctctggtcatctgtggcatcgtgtactggatgcggcggcacacacagaaggcccctaagagaatcagactgccccacatcagatgatga

SEQ ID NO: 56 - DNA, 651 bp, synthetic construct
  source 1..651: mol_type = other DNA
atgggcggagctgctgctagactgggagccgtgatcctgttcgtggttatcgtgggactgcatggcgtgcggggaaagtatcctgccgacggcaaccccgatcctaacgccaatcctaacgtggaccccaacgctaaccccaatgtggacccaaatgccaatccaaatgtcgacccgaacaagaacaatcaaggcaacggccagggccacaacatgcccaacgatcccaacagaaacgtggacgagaacgccaacgccaacagcgccgtgaagaacaacaacaacgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggacgagctggactacgccaacgacatcgagaagaaaatctgcaagatggaaaagtgcagcagcgtgggaggatccggtggtggcggcagcggcggcggacttattgccggcgctgttggaggatctctgctggccgctctggtcatctgtggcatcgtgtactggatgcggcggcacacacagaaggcccctaagagaatcagactgccccacatcagatgatga

SEQ ID NO: 57 - protein, 215 aa, synthetic construct
MGGAAARLGAVILFVVIVGLHGVRGKYPADGNPDPNANPNVDPNANPNVDPNANPNVDPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVGGSGGGGSGGGLIAGAVGGSLLAALVICGIVYWMRRHTQKAPKRIRLPHIR

SEQ ID NO: 58 - RNA, 651 nt, synthetic construct
  source 1..651: mol_type = other RNA
atgggcggagctgctgctagactgggagccgtgatcctgttcgtggttatcgtgggactgcatggcgtgcggggaaagtatcctgccgacggcaaccccgatcctaacgccaatcctaacgtggaccccaacgctaaccccaatgtggacccaaatgccaatccaaatgtcgacccgaacaagaacaatcaaggcaacggccagggccacaacatgcccaacgatcccaacagaaacgtggacgagaacgccaacgccaacagcgccgtgaagaacaacaacaacgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggacgagctggactacgccaacgacatcgagaagaaaatctgcaagatggaaaagtgcagcagcgtgggaggatccggtggtggcggcagcggcggcggacttattgccggcgctgttggaggatctctgctggccgctctggtcatctgtggcatcgtgtactggatgcggcggcacacacagaaggcccctaagagaatcagactgccccacatcagatgatga

SEQ ID NO: 59 - DNA, 795 bp, synthetic construct
  source 1..795: mol_type = other DNA
atgggcggagctgctgctagactgggagccgtgatcctgttcgtggttatcgtgggactgcatggcgtgcggggaaagtatcctgccgacggcaaccccgatcctaacgccaatcctaacgtggaccccaacgctaaccccaatgtggacccaaatgccaatccaaatgtcgatcccaatgcaaacccaaacgtcgacccgaatgctaatcctaatgttgaccctaacgcaaatccgaatgttgatccgaacgctaaccctaatgtcgatcccaacgctaatcccaacgttgacccgaatgcaaaccctaacgtggacccaaacaagaacaatcaaggcaacggccagggccacaacatgcctaacgaccccaacagaaacgtggacgagaacgccaacgccaacagcgccgtgaagaacaacaacaacgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggacgagctggactacgccaacgacatcgagaagaaaatctgcaagatggaaaagtgcagcagcgtgggaggatccggtggtggcggcagcggcggcggacttattgccggcgctgttggaggatctctgctggccgctctggtcatctgtggcatcgtgtactggatgcggcggcacacacagaaggcccctaagagaatcagactgccccacatcagatgatga

SEQ ID NO: 60 - protein, 263 aa, synthetic construct
MGGAAARLGAVILFVVIVGLHGVRGKYPADGNPDPNANPNVDPNANPNVDPNANPNVDPNANPNVDPNANPNVDPNANPNVDPNANPNVDPNANPNVDPNANPNVDPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVGGSGGGGSGGGLIAGAVGGSLLAALVICGIVYWMRRHTQKAPKRIRLPHIR

SEQ ID NO: 61 - RNA, 795 nt, synthetic construct
  source 1..795: mol_type = other RNA
atgggcggagctgctgctagactgggagccgtgatcctgttcgtggttatcgtgggactgcatggcgtgcggggaaagtatcctgccgacggcaaccccgatcctaacgccaatcctaacgtggaccccaacgctaaccccaatgtggacccaaatgccaatccaaatgtcgatcccaatgcaaacccaaacgtcgacccgaatgctaatcctaatgttgaccctaacgcaaatccgaatgttgatccgaacgctaaccctaatgtcgatcccaacgctaatcccaacgttgacccgaatgcaaaccctaacgtggacccaaacaagaacaatcaaggcaacggccagggccacaacatgcctaacgaccccaacagaaacgtggacgagaacgccaacgccaacagcgccgtgaagaacaacaacaacgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggacgagctggactacgccaacgacatcgagaagaaaatctgcaagatggaaaagtgcagcagcgtgggaggatccggtggtggcggcagcggcggcggacttattgccggcgctgttggaggatctctgctggccgctctggtcatctgtggcatcgtgtactggatgcggcggcacacacagaaggcccctaagagaatcagactgccccacatcagatgatga

SEQ ID NO: 62 - DNA, 657 bp, synthetic construct
  source 1..657: mol_type = other DNA
atgggcggagctgctgctagactgggagccgtgatcctgttcgtggttatcgtgggactgcatggcgtgcggggaaagctgaaacagcctgccgatggcaaccccgatcctaacgccaatcctaacgtggaccccaacgctaaccccaatgtggacccaaatgccaatccaaatgtcgacccgaacaagaacaatcaaggcaacggccagggccacaacatgcccaacgatcccaacagaaacgtggacgagaacgccaacgccaacagcgccgtgaagaacaacaacaacgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggacgagctggactacgccaacgacatcgagaagaaaatctgcaagatggaaaagtgcagcagcgtgggaggatccggtggtggcggcagcggcggcggacttattgccggcgctgttggaggatctctgctggccgctctggtcatctgtggcatcgtgtactggatgcggcggcacacacagaaggcccctaagagaatcagactgccccacatcagatgatga

SEQ ID NO: 63 - protein, 217 aa, synthetic construct
MGGAAARLGAVILFVVIVGLHGVRGKLKQPADGNPDPNANPNVDPNANPNVDPNANPNVDPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVGGSGGGGSGGGLIAGAVGGSLLAALVICGIVYWMRRHTQKAPKRIRLPHIR

SEQ ID NO: 64 - RNA, 657 nt, synthetic construct
  source 1..657: mol_type = other RNA
atgggcggagctgctgctagactgggagccgtgatcctgttcgtggttatcgtgggactgcatggcgtgcggggaaagctgaaacagcctgccgatggcaaccccgatcctaacgccaatcctaacgtggaccccaacgctaaccccaatgtggacccaaatgccaatccaaatgtcgacccgaacaagaacaatcaaggcaacggccagggccacaacatgcccaacgatcccaacagaaacgtggacgagaacgccaacgccaacagcgccgtgaagaacaacaacaacgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggacgagctggactacgccaacgacatcgagaagaaaatctgcaagatggaaaagtgcagcagcgtgggaggatccggtggtggcggcagcggcggcggacttattgccggcgctgttggaggatctctgctggccgctctggtcatctgtggcatcgtgtactggatgcggcggcacacacagaaggcccctaagagaatcagactgccccacatcagatgatga

SEQ ID NO: 65 - DNA, 801 bp, synthetic construct
  source 1..801: mol_type = other DNA
atgggcggagctgctgctagactgggagccgtgatcctgttcgtggttatcgtgggactgcatggcgtgcggggaaagctgaaacagcctgccgatggcaaccccgatcctaacgccaatcctaacgtggaccccaacgctaaccccaatgtggacccaaatgccaatccaaatgtcgatcccaatgcaaacccaaacgtcgacccgaatgctaatcctaatgttgaccctaacgcaaatccgaatgttgatccgaacgctaaccctaatgtcgatcccaacgctaatcccaacgttgacccgaatgcaaaccctaacgtggacccaaacaagaacaatcaaggcaacggccagggccacaacatgcctaacgaccccaacagaaacgtggacgagaacgccaacgccaacagcgccgtgaagaacaacaacaacgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggacgagctggactacgccaacgacatcgagaagaaaatctgcaagatggaaaagtgcagcagcgtgggaggatccggtggtggcggcagcggcggcggacttattgccggcgctgttggaggatctctgctggccgctctggtcatctgtggcatcgtgtactggatgcggcggcacacacagaaggcccctaagagaatcagactgccccacatcagatgatga

SEQ ID NO: 66 - protein, 265 aa, synthetic construct
MGGAAARLGAVILFVVIVGLHGVRGKLKQPADGNPDPNANPNVDPNANPNVDPNANPNVDPNANPNVDPNANPNVDPNANPNVDPNANPNVDPNANPNVDPNANPNVDPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVGGSGGGGSGGGLIAGAVGGSLLAALVICGIVYWMRRHTQKAPKRIRLPHIR

SEQ ID NO: 67 - RNA, 801 nt, synthetic construct
  source 1..801: mol_type = other RNA
atgggcggagctgctgctagactgggagccgtgatcctgttcgtggttatcgtgggactgcatggcgtgcggggaaagctgaaacagcctgccgatggcaaccccgatcctaacgccaatcctaacgtggaccccaacgctaaccccaatgtggacccaaatgccaatccaaatgtcgatcccaatgcaaacccaaacgtcgacccgaatgctaatcctaatgttgaccctaacgcaaatccgaatgttgatccgaacgctaaccctaatgtcgatcccaacgctaatcccaacgttgacccgaatgcaaaccctaacgtggacccaaacaagaacaatcaaggcaacggccagggccacaacatgcctaacgaccccaacagaaacgtggacgagaacgccaacgccaacagcgccgtgaagaacaacaacaacgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggacgagctggactacgccaacgacatcgagaagaaaatctgcaagatggaaaagtgcagcagcgtgggaggatccggtggtggcggcagcggcggcggacttattgccggcgctgttggaggatctctgctggccgctctggtcatctgtggcatcgtgtactggatgcggcggcacacacagaaggcccctaagagaatcagactgccccacatcagatgatga

SEQ ID NO: 68 - DNA, 663 bp, synthetic construct
  source 1..663: mol_type = other DNA
atgggcggagctgctgctagactgggagccgtgatcctgttcgtggttatcgtgggactgcacggcgtgcggggcaaatatgctgctaaacagcccgccgacggcaaccccgatcctaacgctaatcccaacgtggaccccaacgccaatcctaatgtcgacccaaacgcaaacccaaacgtcgaccctaacaagaacaatcaaggcaacggccagggccacaacatgcccaacgatcccaacagaaacgtggacgagaacgccaatgccaacagcgccgtgaagaacaacaacaacgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggacgagctggactacgccaacgacatcgagaagaaaatctgcaagatggaaaagtgcagcagcgtgggaggatccggtggtggcggcagcggcggcggacttattgccggcgctgttggaggatctctgctggccgctctggtcatctgtggcatcgtgtactggatgcggcggcacacacagaaggcccctaagagaatcagactgccccacatcagatgatga

SEQ ID NO: 69 - protein, 219 aa, synthetic construct
MGGAAARLGAVILFVVIVGLHGVRGKYAAKQPADGNPDPNANPNVDPNANPNVDPNANPNVDPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVGGSGGGGSGGGLIAGAVGGSLLAALVICGIVYWMRRHTQKAPKRIRLPHIR

SEQ ID NO: 70 - RNA, 663 nt, synthetic construct
  source 1..663: mol_type = other RNA
atgggcggagctgctgctagactgggagccgtgatcctgttcgtggttatcgtgggactgcacggcgtgcggggcaaatatgctgctaaacagcccgccgacggcaaccccgatcctaacgctaatcccaacgtggaccccaacgccaatcctaatgtcgacccaaacgcaaacccaaacgtcgaccctaacaagaacaatcaaggcaacggccagggccacaacatgcccaacgatcccaacagaaacgtggacgagaacgccaatgccaacagcgccgtgaagaacaacaacaacgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggacgagctggactacgccaacgacatcgagaagaaaatctgcaagatggaaaagtgcagcagcgtgggaggatccggtggtggcggcagcggcggcggacttattgccggcgctgttggaggatctctgctggccgctctggtcatctgtggcatcgtgtactggatgcggcggcacacacagaaggcccctaagagaatcagactgccccacatcagatgatga

SEQ ID NO: 71 - DNA, 807 bp, synthetic construct
  source 1..807: mol_type = other DNA
atgggcggagctgctgctagactgggagccgtgatcctgttcgtggttatcgtgggactgcacggcgtgcggggcaaatatgctgctaaacagcccgccgacggcaaccccgatcctaacgctaatcccaacgtggaccccaacgccaatcctaatgtcgacccaaacgcaaacccaaacgtcgaccctaatgctaaccccaatgttgatcccaatgcaaacccgaatgtggaccctaacgcaaatccaaatgttgatccgaacgccaatccgaacgttgacccgaatgctaatccaaacgtggacccaaatgcaaaccccaacgtggacccaaacaagaacaatcaaggcaacggccagggccacaacatgcccaacgatcccaacagaaatgtggacgagaatgccaatgccaacagcgccgtgaagaacaacaacaacgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggacgagctggactacgccaacgacatcgagaagaaaatctgcaagatggaaaagtgcagcagcgtgggaggatccggtggtggcggcagcggcggcggacttattgccggcgctgttggaggatctctgctggccgctctggtcatctgtggcatcgtgtactggatgcggcggcacacacagaaggcccctaagagaatcagactgccccacatcagatgatga

SEQ ID NO: 72 - protein, 267 aa, synthetic construct
MGGAAARLGAVILFVVIVGLHGVRGKYAAKQPADGNPDPNANPNVDPNANPNVDPNANPNVDPNANPNVDPNANPNVDPNANPNVDPNANPNVDPNANPNVDPNANPNVDPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVGGSGGGGSGGGLIAGAVGGSLLAALVICGIVYWMRRHTQKAPKRIRLPHIR

SEQ ID NO: 73 - RNA, 807 nt, synthetic construct
  source 1..807: mol_type = other RNA
atgggcggagctgctgctagactgggagccgtgatcctgttcgtggttatcgtgggactgcacggcgtgcggggcaaatatgctgctaaacagcccgccgacggcaaccccgatcctaacgctaatcccaacgtggaccccaacgccaatcctaatgtcgacccaaacgcaaacccaaacgtcgaccctaatgctaaccccaatgttgatcccaatgcaaacccgaatgtggaccctaacgcaaatccaaatgttgatccgaacgccaatccgaacgttgacccgaatgctaatccaaacgtggacccaaatgcaaaccccaacgtggacccaaacaagaacaatcaaggcaacggccagggccacaacatgcccaacgatcccaacagaaatgtggacgagaatgccaatgccaacagcgccgtgaagaacaacaacaacgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggacgagctggactacgccaacgacatcgagaagaaaatctgcaagatggaaaagtgcagcagcgtgggaggatccggtggtggcggcagcggcggcggacttattgccggcgctgttggaggatctctgctggccgctctggtcatctgtggcatcgtgtactggatgcggcggcacacacagaaggcccctaagagaatcagactgccccacatcagatgatga

SEQ ID NO: 74 - DNA, 642 bp, synthetic construct
  source 1..642: mol_type = other DNA
atgggcggagctgctgctagactgggagccgtgatcctgttcgtggttatcgtgggactgcatggcgtcagaggcgccgatggaaaccccgatcctaacgccaatcctaacgtggaccccaacgctaaccccaatgtggacccaaatgccaatccaaatgtcgacccgaacaagaacaatcaaggcaacggccagggccacaacatgcccaacgatcccaacagaaacgtggacgagaacgccaacgccaacagcgccgtgaagaacaacaacaacgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggacgagctggactacgccaacgacatcgagaagaaaatctgcaagatggaaaagtgcagcagcgtgggaggatccggtggtggcggcagcggcggcggacttattgccggcgctgttggaggatctctgctggccgctctggtcatctgtggcatcgtgtactggatgcggcggcacacacagaaggcccctaagagaatcagactgccccacatcagatgatga

SEQ ID NO: 75 - protein, 212 aa, synthetic construct
MGGAAARLGAVILFVVIVGLHGVRGADGNPDPNANPNVDPNANPNVDPNANPNVDPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVGGSGGGGSGGGLIAGAVGGSLLAALVICGIVYWMRRHTQKAPKRIRLPHIR

SEQ ID NO: 76 - RNA, 642 nt, synthetic construct
  source 1..642: mol_type = other RNA
atgggcggagctgctgctagactgggagccgtgatcctgttcgtggttatcgtgggactgcatggcgtcagaggcgccgatggaaaccccgatcctaacgccaatcctaacgtggaccccaacgctaaccccaatgtggacccaaatgccaatccaaatgtcgacccgaacaagaacaatcaaggcaacggccagggccacaacatgcccaacgatcccaacagaaacgtggacgagaacgccaacgccaacagcgccgtgaagaacaacaacaacgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggacgagctggactacgccaacgacatcgagaagaaaatctgcaagatggaaaagtgcagcagcgtgggaggatccggtggtggcggcagcggcggcggacttattgccggcgctgttggaggatctctgctggccgctctggtcatctgtggcatcgtgtactggatgcggcggcacacacagaaggcccctaagagaatcagactgccccacatcagatgatga

SEQ ID NO: 77 - DNA, 786 bp, synthetic construct
  source 1..786: mol_type = other DNA
atgggcggagctgctgctagactgggagccgtgatcctgttcgtggttatcgtgggactgcatggcgtcagaggcgccgatggaaaccccgatcctaacgccaatcctaacgtggaccccaacgctaaccccaatgtggacccaaatgccaatccaaatgtcgatcccaatgcaaacccaaacgtcgacccgaatgctaatcctaatgttgaccctaacgcaaatccgaatgttgatccgaacgctaaccctaatgtcgatcccaacgctaatcccaacgttgacccgaatgcaaaccctaacgtggacccaaacaagaacaatcaaggcaacggccagggccacaacatgcctaacgaccccaacagaaacgtggacgagaacgccaacgccaacagcgccgtgaagaacaacaacaacgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggacgagctggactacgccaacgacatcgagaagaaaatctgcaagatggaaaagtgcagcagcgtgggaggatccggtggtggcggcagcggcggcggacttattgccggcgctgttggaggatctctgctggccgctctggtcatctgtggcatcgtgtactggatgcggcggcacacacagaaggcccctaagagaatcagactgccccacatcagatgatga

SEQ ID NO: 78 - protein, 260 aa, synthetic construct
MGGAAARLGAVILFVVIVGLHGVRGADGNPDPNANPNVDPNANPNVDPNANPNVDPNANPNVDPNANPNVDPNANPNVDPNANPNVDPNANPNVDPNANPNVDPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVGGSGGGGSGGGLIAGAVGGSLLAALVICGIVYWMRRHTQKAPKRIRLPHIR

SEQ ID NO: 79 - RNA, 786 nt, synthetic construct
  source 1..786: mol_type = other RNA
atgggcggagctgctgctagactgggagccgtgatcctgttcgtggttatcgtgggactgcatggcgtcagaggcgccgatggaaaccccgatcctaacgccaatcctaacgtggaccccaacgctaaccccaatgtggacccaaatgccaatccaaatgtcgatcccaatgcaaacccaaacgtcgacccgaatgctaatcctaatgttgaccctaacgcaaatccgaatgttgatccgaacgctaaccctaatgtcgatcccaacgctaatcccaacgttgacccgaatgcaaaccctaacgtggacccaaacaagaacaatcaaggcaacggccagggccacaacatgcctaacgaccccaacagaaacgtggacgagaacgccaacgccaacagcgccgtgaagaacaacaacaacgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggacgagctggactacgccaacgacatcgagaagaaaatctgcaagatggaaaagtgcagcagcgtgggaggatccggtggtggcggcagcggcggcggacttattgccggcgctgttggaggatctctgctggccgctctggtcatctgtggcatcgtgtactggatgcggcggcacacacagaaggcccctaagagaatcagactgccccacatcagatgatga

SEQ ID NO: 80 - DNA, 837 bp, synthetic construct
  source 1..837: mol_type = other DNA
atgggcggagctgctgctagactgggagccgtgatcctgttcgtggttatcgtgggactgcacggcgtgcgcggagaggataatgagaagctgagaaagcccaagcacaagaagctgaagcagcccgccgacggcaaccccgatcctaacgctaatcccaacgtggaccccaacgccaatcctaatgtcgacccaaacgcaaacccaaacgtcgaccctaatgctaaccccaatgttgatcccaatgcaaacccgaatgtggaccctaacgcaaatccaaatgttgatccgaacgccaatccgaacgttgacccgaatgctaatccaaacgtggacccaaatgcaaaccccaacgtggacccaaacaagaacaatcaaggcaacggccagggccacaacatgcccaacgatcccaacagaaatgtggacgagaatgccaatgccaacagcgccgtgaagaacaacaacaacgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggacgagctggactacgccaacgacatcgagaagaaaatctgcaagatggaaaagtgcagcagcgtgggaggatccggtggtggcggcagcggcggcggacttattgccggcgctgttggaggatctctgctggccgctctggtcatctgtggcatcgtgtactggatgcggcggcacacacagaaggcccctaagagaatcagactgccccacatcagatgatga

SEQ ID NO: 81 - protein, 277 aa, synthetic construct
MGGAAARLGAVILFVVIVGLHGVRGEDNEKLRKPKHKKLKQPADGNPDPNANPNVDPNANPNVDPNANPNVDPNANPNVDPNANPNVDPNANPNVDPNANPNVDPNANPNVDPNANPNVDPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVGGSGGGGSGGGLIAGAVGGSLLAALVICGIVYWMRRHTQKAPKRIRLPHIR

SEQ ID NO: 82 - RNA, 837 nt, synthetic construct
  source 1..837: mol_type = other RNA
atgggcggagctgctgctagactgggagccgtgatcctgttcgtggttatcgtgggactgcacggcgtgcgcggagaggataatgagaagctgagaaagcccaagcacaagaagctgaagcagcccgccgacggcaaccccgatcctaacgctaatcccaacgtggaccccaacgccaatcctaatgtcgacccaaacgcaaacccaaacgtcgaccctaatgctaaccccaatgttgatcccaatgcaaacccgaatgtggaccctaacgcaaatccaaatgttgatccgaacgccaatccgaacgttgacccgaatgctaatccaaacgtggacccaaatgcaaaccccaacgtggacccaaacaagaacaatcaaggcaacggccagggccacaacatgcccaacgatcccaacagaaatgtggacgagaatgccaatgccaacagcgccgtgaagaacaacaacaacgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggacgagctggactacgccaacgacatcgagaagaaaatctgcaagatggaaaagtgcagcagcgtgggaggatccggtggtggcggcagcggcggcggacttattgccggcgctgttggaggatctctgctggccgctctggtcatctgtggcatcgtgtactggatgcggcggcacacacagaaggcccctaagagaatcagactgccccacatcagatgatga

SEQ ID NO: 83 - DNA, 837 bp, synthetic construct
  source 1..837: mol_type = other DNA
atgggcggagctgctgctagactgggagccgtgatcctgttcgtggttatcgtgggactgcacggcgtgcgcggagaggataatgagaagctgagaaagcccaagcacaaggccgccaagcagcctgccgatggcaaccctgatcctaacgccaatcctaacgtggaccccaacgctaaccccaatgtggacccaaatgccaatccaaatgtcgatcccaatgcaaacccaaacgtcgacccgaatgctaatcctaatgttgaccctaacgcaaatccgaatgttgatccgaacgctaaccctaatgtcgatcccaacgctaatcccaacgttgacccgaatgcaaaccctaacgtggacccaaacaagaacaatcaaggcaacggccagggccacaacatgcctaacgaccccaacagaaacgtggacgagaacgccaacgccaacagcgccgtgaagaacaacaacaacgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggacgagctggactacgccaacgacatcgagaagaaaatctgcaagatggaaaagtgcagcagcgtgggaggatccggtggtggcggcagcggcggcggacttattgccggcgctgttggaggatctctgctggccgctctggtcatctgtggcatcgtgtactggatgcggcggcacacacagaaggcccctaagagaatcagactgccccacatcagatgatga

SEQ ID NO: 84 - protein, 277 aa, synthetic construct
MGGAAARLGAVILFVVIVGLHGVRGEDNEKLRKPKHKAAKQPADGNPDPNANPNVDPNANPNVDPNANPNVDPNANPNVDPNANPNVDPNANPNVDPNANPNVDPNANPNVDPNANPNVDPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVGGSGGGGSGGGLIAGAVGGSLLAALVICGIVYWMRRHTQKAPKRIRLPHIR

SEQ ID NO: 85 - RNA, 837 nt, synthetic construct
  source 1..837: mol_type = other RNA
atgggcggagctgctgctagactgggagccgtgatcctgttcgtggttatcgtgggactgcacggcgtgcgcggagaggataatgagaagctgagaaagcccaagcacaaggccgccaagcagcctgccgatggcaaccctgatcctaacgccaatcctaacgtggaccccaacgctaaccccaatgtggacccaaatgccaatccaaatgtcgatcccaatgcaaacccaaacgtcgacccgaatgctaatcctaatgttgaccctaacgcaaatccgaatgttgatccgaacgctaaccctaatgtcgatcccaacgctaatcccaacgttgacccgaatgcaaaccctaacgtggacccaaacaagaacaatcaaggcaacggccagggccacaacatgcctaacgaccccaacagaaacgtggacgagaacgccaacgccaacagcgccgtgaagaacaacaacaacgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggacgagctggactacgccaacgacatcgagaagaaaatctgcaagatggaaaagtgcagcagcgtgggaggatccggtggtggcggcagcggcggcggacttattgccggcgctgttggaggatctctgctggccgctctggtcatctgtggcatcgtgtactggatgcggcggcacacacagaaggcccctaagagaatcagactgccccacatcagatgatga

SEQ ID NO: 86 - DNA, 1143 bp, synthetic construct
  source 1..1143: mol_type = other DNA
atgggcggagctgctgctagactgggagccgtgatcctgttcgtggttatcgtgggactgcacggcgtgcgcggagaggataatgagaagctgagaaagcccaagcacaagaagctgaagcagcccgccgacggcaaccccgatcctaacgctaatcccaacgtggaccccaacgccaatcctaatgttgaccctggcggatctggcggcggaggttctggtggcgaggacaacgagaaactgcggaagcctaaacacaagaaactcaaacagcctgctgacgggaatcctgatccaaacgcaaaccccaatgtcgaccctaatgcaaacccaaacgttgacccaggtggcagcggaggcggaggaagtggtggcgaagataacgaaaagcttcgcaaaccgaaacataagaagttgaaacagccggcagatggaaatccagatccgaatgccaatccgaacgtcgacccaaacgctaacccaaatgtggacccaggcggtagtggcggtggtggctctggcggggaagataacgagaaactcagaaagccgaaacacaaaaagcttaaacaacccgccgatgggaacccagatccaaatgctaaccctaatgttgatcccaacgctaaccctaacgtcgacccgaatgctaatcctaacgccaatccgaacaagaacaatcaaggcaacggccagggccacaacatgcccaacgatcccaacagaaacgtggacgagaacgccaatgccaacagcgccgtgaagaacaacaacaacgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggacgagctggactacgccaacgacatcgagaagaaaatctgcaagatggaaaagtgcagcagcgtgggaggatccggtggtggcggcagcggcggcggacttattgccggcgctgttggaggatctctgctggccgctctggtcatctgtggcatcgtgtactggatgcggcggcacacacagaaggcccctaagagaatcagactgccccacatcagatgatga

SEQ ID NO: 87 - protein, 379 aa, synthetic construct
MGGAAARLGAVILFVVIVGLHGVRGEDNEKLRKPKHKKLKQPADGNPDPNANPNVDPNANPNVDPGGSGGGGSGGEDNEKLRKPKHKKLKQPADGNPDPNANPNVDPNANPNVDPGGSGGGGSGGEDNEKLRKPKHKKLKQPADGNPDPNANPNVDPNANPNVDPGGSGGGGSGGEDNEKLRKPKHKKLKQPADGNPDPNANPNVDPNANPNVDPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVGGSGGGGSGGGLIAGAVGGSLLAALVICGIVYWMRRHTQKAPKRIRLPHIR

SEQ ID NO: 88 - RNA, 1143 nt, synthetic construct
  source 1..1143: mol_type = other RNA
atgggcggagctgctgctagactgggagccgtgatcctgttcgtggttatcgtgggactgcacggcgtgcgcggagaggataatgagaagctgagaaagcccaagcacaagaagctgaagcagcccgccgacggcaaccccgatcctaacgctaatcccaacgtggaccccaacgccaatcctaatgttgaccctggcggatctggcggcggaggttctggtggcgaggacaacgagaaactgcggaagcctaaacacaagaaactcaaacagcctgctgacgggaatcctgatccaaacgcaaaccccaatgtcgaccctaatgcaaacccaaacgttgacccaggtggcagcggaggcggaggaagtggtggcgaagataacgaaaagcttcgcaaaccgaaacataagaagttgaaacagccggcagatggaaatccagatccgaatgccaatccgaacgtcgacccaaacgctaacccaaatgtggacccaggcggtagtggcggtggtggctctggcggggaagataacgagaaactcagaaagccgaaacacaaaaagcttaaacaacccgccgatgggaacccagatccaaatgctaaccctaatgttgatcccaacgctaaccctaacgtcgacccgaatgctaatcctaacgccaatccgaacaagaacaatcaaggcaacggccagggccacaacatgcccaacgatcccaacagaaacgtggacgagaacgccaatgccaacagcgccgtgaagaacaacaacaacgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggacgagctggactacgccaacgacatcgagaagaaaatctgcaagatggaaaagtgcagcagcgtgggaggatccggtggtggcggcagcggcggcggacttattgccggcgctgttggaggatctctgctggccgctctggtcatctgtggcatcgtgtactggatgcggcggcacacacagaaggcccctaagagaatcagactgccccacatcagatgatga

SEQ ID NO: 89 - DNA, 1305 bp, synthetic construct
  source 1..1305: mol_type = other DNA
atgggcggagctgctgctagactgggagccgtgatcctgttcgtggttatcgtgggactgcacggcgtgcggggacaagagtatcagtgttacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcagcccgccgacggcaatcccgatcctaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcctaacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagcagcgtgggaggatccggtggtggcggcagcggcggcggacttattgccggcgctgttggaggatctctgctggccgctctggtcatctgtggcatcgtgtactggatgcggcggcacacacagaaggcccctaagagaatcagactgccccacatcagatgatga

SEQ ID NO: 90 - protein, 433 aa, synthetic construct
MGGAAARLGAVILFVVIVGLHGVRGQEYQCYGSSSNTRVLNELNYDNAGTNLYNELEMNYYGKQENWYSLKKNSRSLGENDDGNNEDNEKLRKPKHKKLKQPADGNPDPNANPNVDPNANPNVDPNANPNVDPNANPNANPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVGGSGGGGSGGGLIAGAVGGSLLAALVICGIVYWMRRHTQKAPKRIRLPHIR

SEQ ID NO: 91 - RNA, 1305 nt, synthetic construct
  source 1..1305: mol_type = other RNA
atgggcggagctgctgctagactgggagccgtgatcctgttcgtggttatcgtgggactgcacggcgtgcggggacaagagtatcagtgttacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcagcccgccgacggcaatcccgatcctaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcctaacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagcagcgtgggaggatccggtggtggcggcagcggcggcggacttattgccggcgctgttggaggatctctgctggccgctctggtcatctgtggcatcgtgtactggatgcggcggcacacacagaaggcccctaagagaatcagactgccccacatcagatgatga

SEQ ID NO: 92 - DNA, 1152 bp, synthetic construct
  source 1..1152: mol_type = other DNA
atgggcggagctgctgctagactgggagccgtgatcctgttcgtggttatcgtgggactgcacggcgtgcggggacaagagtatcagtgttacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcagcccgccgacggcaatcccgatcctaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcctaacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagcagcgtgtgatga

SEQ ID NO: 93 - protein, 382 aa, synthetic construct
MGGAAARLGAVILFVVIVGLHGVRGQEYQCYGSSSNTRVLNELNYDNAGTNLYNELEMNYYGKQENWYSLKKNSRSLGENDDGNNEDNEKLRKPKHKKLKQPADGNPDPNANPNVDPNANPNVDPNANPNVDPNANPNANPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSV

SEQ ID NO: 94 - RNA, 1152 nt, synthetic construct
  source 1..1152: mol_type = other RNA
atgggcggagctgctgctagactgggagccgtgatcctgttcgtggttatcgtgggactgcacggcgtgcggggacaagagtatcagtgttacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcagcccgccgacggcaatcccgatcctaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcctaacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagcagcgtgtgatga

SEQ ID NO: 95 - DNA, 744 bp, synthetic construct
  source 1..744: mol_type = other DNA
atgggcggagctgctgctagactgggagccgtgatcctgttcgtggttatcgtgggactgcacggcgtgcgcggagaggataatgagaagctgagaaagcccaagcacaagaagctgaagcagcccgccgacggcaaccccgatcctaacgctaatcccaacgtggaccccaacgccaatcctaatgtcgacccaaacgcaaacccaaacgtcgaccctaatgctaaccccaatgttgatcccaatgcaaacccgaatgtggaccctaacgcaaatccaaatgttgatccgaacgccaatccgaacgttgacccgaatgctaatccaaacgtggacccaaatgcaaaccccaacgtggacccaaacaagaacaatcaaggcaacggccagggccacaacatgcccaacgatcccaacagaaatgtggacgagaatgccaatgccaacagcgccgtgaagaacaacaacaacgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggacgagctggactacgccaacgacatcgagaagaaaatctgcaagatggaaaagtgcagcagcgtgttcaacgtggtcaacagcagcatcggcctgattatggtgctgagcttcctgttcctgaactgataa

SEQ ID NO: 96 - protein, 246 aa, synthetic construct
MGGAAARLGAVILFVVIVGLHGVRGEDNEKLRKPKHKKLKQPADGNPDPNANPNVDPNANPNVDPNANPNVDPNANPNVDPNANPNVDPNANPNVDPNANPNVDPNANPNVDPNANPNVDPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVFNVVNSSIGLIMVLSFLFLN

SEQ ID NO: 97 - RNA, 744 nt, synthetic construct
  source 1..744: mol_type = other RNA
atgggcggagctgctgctagactgggagccgtgatcctgttcgtggttatcgtgggactgcacggcgtgcgcggagaggataatgagaagctgagaaagcccaagcacaagaagctgaagcagcccgccgacggcaaccccgatcctaacgctaatcccaacgtggaccccaacgccaatcctaatgtcgacccaaacgcaaacccaaacgtcgaccctaatgctaaccccaatgttgatcccaatgcaaacccgaatgtggaccctaacgcaaatccaaatgttgatccgaacgccaatccgaacgttgacccgaatgctaatccaaacgtggacccaaatgcaaaccccaacgtggacccaaacaagaacaatcaaggcaacggccagggccacaacatgcccaacgatcccaacagaaatgtggacgagaatgccaatgccaacagcgccgtgaagaacaacaacaacgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggacgagctggactacgccaacgacatcgagaagaaaatctgcaagatggaaaagtgcagcagcgtgttcaacgtggtcaacagcagcatcggcctgattatggtgctgagcttcctgttcctgaactgataa

SEQ ID NO: 98 - DNA, 723 bp, synthetic construct
  source 1..723: mol_type = other DNA
atgatgcggaagctggccatcctgagcgtgtccagcttcctgtttgtggaagccgaggacaacgagaagctgcggaagcccaagcacaagaagctgaagcagcccgccgacggcaaccccgatcctaacgctaatcccaacgtggaccccaacgccaatcctaatgtcgacccaaacgcaaacccaaacgtcgaccctaatgctaaccccaatgttgatcccaatgcaaacccgaatgtggaccctaacgcaaatccaaatgttgatccgaacgccaatccgaacgttgacccgaatgctaatccaaacgtggacccaaatgcaaaccccaacgtggacccaaacaagaacaatcaaggcaacggccagggccacaacatgcccaacgatcccaacagaaatgtggacgagaatgccaatgccaacagcgccgtgaagaacaacaacaacgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggacgagctggactacgccaacgacatcgagaagaaaatctgcaagatggaaaagtgcagcagcgtgttcaacgtggtcaacagcagcatcggcctgattatggtgctgtccttcctgttcctgaactgataa

SEQ ID NO: 99 - protein, 239 aa, synthetic construct
MMRKLAILSVSSFLFVEAEDNEKLRKPKHKKLKQPADGNPDPNANPNVDPNANPNVDPNANPNVDPNANPNVDPNANPNVDPNANPNVDPNANPNVDPNANPNVDPNANPNVDPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVFNVVNSSIGLIMVLSFLFLN

SEQ ID NO: 100 - RNA, 723 nt, synthetic construct
  source 1..723: mol_type = other RNA
atgatgcggaagctggccatcctgagcgtgtccagcttcctgtttgtggaagccgaggacaacgagaagctgcggaagcccaagcacaagaagctgaagcagcccgccgacggcaaccccgatcctaacgctaatcccaacgtggaccccaacgccaatcctaatgtcgacccaaacgcaaacccaaacgtcgaccctaatgctaaccccaatgttgatcccaatgcaaacccgaatgtggaccctaacgcaaatccaaatgttgatccgaacgccaatccgaacgttgacccgaatgctaatccaaacgtggacccaaatgcaaaccccaacgtggacccaaacaagaacaatcaaggcaacggccagggccacaacatgcccaacgatcccaacagaaatgtggacgagaatgccaatgccaacagcgccgtgaagaacaacaacaacgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggacgagctggactacgccaacgacatcgagaagaaaatctgcaagatggaaaagtgcagcagcgtgttcaacgtggtcaacagcagcatcggcctgattatggtgctgtccttcctgttcctgaactgataa

SEQ ID NO: 101 - skipped

SEQ ID NO: 102 - protein, 8 aa, synthetic construct
NANPNVDP

SEQ ID NO: 103 - skipped

SEQ ID NO: 104 - DNA, 375 bp, unidentified
  source 1..375: mol_type = other DNA; note = Plasmodium falciparum
aacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagcagcgtgttcaacgtggtcaacagcagcatcggcctgattatggtgctgtccttcctgttcctgaac

SEQ ID NO: 105 - protein, 125 aa, unidentified
  source 1..125: note = Plasmodium falciparum
NKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVFNVVNSSIGLIMVLSFLFLN

SEQ ID NO: 106 - RNA, 375 nt, unidentified
  source 1..375: mol_type = other RNA; note = Plasmodium falciparum
aacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagcagcgtgttcaacgtggtcaacagcagcatcggcctgattatggtgctgtccttcctgttcctgaac

SEQ ID NO: 107 - DNA, 309 bp, unidentified
  source 1..309: mol_type = other DNA; note = Plasmodium falciparum
aacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagc

SEQ ID NO: 108 - protein, 103 aa, unidentified
  source 1..103: note = Plasmodium falciparum
NKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCS

SEQ ID NO: 109 - RNA, 309 nt, unidentified
  source 1..309: mol_type = other RNA; note = Plasmodium falciparum
aacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagc

SEQ ID NO: 110 - DNA, 42 bp, unidentified
  source 1..42: mol_type = other DNA; note = Plasmodium falciparum
aagcacatcaaagagtacctgaacaagatccagaacagcctg

SEQ ID NO: 111 - protein, 14 aa, unidentified
  source 1..14: note = Plasmodium falciparum
KHIKEYLNKIQNSL

SEQ ID NO: 112 - RNA, 42 nt, unidentified
  source 1..42: mol_type = other RNA; note = Plasmodium falciparum
aagcacatcaaagagtacctgaacaagatccagaacagcctg

SEQ ID NO: 113 - DNA, 36 bp, unidentified
  source 1..36: mol_type = other DNA; note = Plasmodium falciparum
aacaagcctaaggatgagctggactacgccaatgac

SEQ ID NO: 114 - protein, 12 aa, unidentified
  source 1..12: note = Plasmodium falciparum
NKPKDELDYAND

SEQ ID NO: 115 - RNA, 36 nt, unidentified
  source 1..36: mol_type = other RNA; note = Plasmodium falciparum
aacaagcctaaggatgagctggactacgccaatgac

SEQ ID NO: 116 - DNA, 147 bp, unidentified
  source 1..147: mol_type = other DNA; note = Plasmodium falciparum
agcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgc

SEQ ID NO: 117 - protein, 49 aa, unidentified
  source 1..49: note = Plasmodium falciparum
SLSTEWSPCSVTCGNGIQVRIKPGSANKPKDELDYANDIEKKICKMEKC

SEQ ID NO: 118 - RNA, 147 nt, unidentified
  source 1..147: mol_type = other RNA; note = Plasmodium falciparum
agcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgc

SEQ ID NO: 119 - DNA, 42 bp, synthetic construct
  source 1..42: mol_type = other DNA
atcgagaagaaaatctgcaagatggaaaagtgcagcagcgtg

SEQ ID NO: 120 - protein, 14 aa, synthetic construct
IEKKICKMEKCSSV

SEQ ID NO: 121 - RNA, 42 nt, synthetic construct
  source 1..42: mol_type = other RNA
atcgagaagaaaatctgcaagatggaaaagtgcagcagcgtg

SEQ ID NO: 122 - DNA, 504 bp, unidentified
  source 1..504: mol_type = other DNA; note = Plasmodium falciparum
aacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcct

SEQ ID NO: 123 - protein, 168 aa, unidentified
  source 1..168: note = Plasmodium falciparum
NANPNVDPNANPNVDPNANPNVDPNANPNANPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANP

SEQ ID NO: 124 - RNA, 504 nt, unidentified
  source 1..504: mol_type = other RNA; note = Plasmodium falciparum
aacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcct

SEQ ID NO: 125 - DNA, 36 bp, unidentified
  source 1..36: mol_type = other DNA; note = Plasmodium falciparum
aagctgaagcagcccgccgacggcaatcccgatcct

SEQ ID NO: 126 - protein, 12 aa, unidentified
  source 1..12: note = Plasmodium falciparum
KLKQPADGNPDP

SEQ ID NO: 127 - RNA, 36 nt, unidentified
  source 1..36: mol_type = other RNA; note = Plasmodium falciparum
aagctgaagcagcccgccgacggcaatcccgatcct

SEQ ID NO: 128 - DNA, 24 bp, unidentified
  source 1..24: mol_type = other DNA; note = Plasmodium falciparum
cccgccgacggcaatcccgatcct

SEQ ID NO: 129 - protein, 8 aa, unidentified
  source 1..8: note = Plasmodium falciparum
PADGNPDP

SEQ ID NO: 130 - RNA, 24 nt, unidentified
  source 1..24: mol_type = other RNA; note = Plasmodium falciparum
cccgccgacggcaatcccgatcct

SEQ ID NO: 131 - DNA, 21 bp, unidentified
  source 1..21: mol_type = other DNA; note = Plasmodium falciparum
gccgacggcaatcccgatcct

SEQ ID NO: 132 - protein, 7 aa, unidentified
  source 1..7: note = Plasmodium falciparum
ADGNPDP

SEQ ID NO: 133 - RNA, 21 nt, unidentified
  source 1..21: mol_type = other RNA; note = Plasmodium falciparum
gccgacggcaatcccgatcct

SEQ ID NO: 134 - DNA, 36 bp, unidentified
  source 1..36: mol_type = other DNA; note = Plasmodium falciparum
gaggacaacgagaagctgcggaagcccaagcacaag

SEQ ID NO: 135 - protein, 12 aa, unidentified
  source 1..12: note = Plasmodium falciparum
EDNEKLRKPKHK

SEQ ID NO: 136 - RNA, 36 nt, unidentified
  source 1..36: mol_type = other RNA; note = Plasmodium falciparum
gaggacaacgagaagctgcggaagcccaagcacaag

SEQ ID NO: 137 - DNA, 186 bp, unidentified
  source 1..186: mol_type = other DNA; note = Plasmodium falciparum
ctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaac

SEQ ID NO: 138 - protein, 62 aa, unidentified
  source 1..62: note = Plasmodium falciparum
LFQEYQCYGSSSNTRVLNELNYDNAGTNLYNELEMNYYGKQENWYSLKKNSRSLGENDDGNN

SEQ ID NO: 139 - RNA, 186 nt, unidentified
  source 1..186: mol_type = other RNA; note = Plasmodium falciparum
ctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaac

SEQ ID NO: 140 - skipped

SEQ ID NO: 141 - protein, 4 aa, synthetic construct
NPNA

SEQ ID NO: 142-143 - skipped (2 entries)

SEQ ID NO: 144 - protein, 4 aa, synthetic construct
NVDP

SEQ ID NO: 145-146 - skipped (2 entries)

SEQ ID NO: 147 - protein, 4 aa, synthetic construct
NANP

SEQ ID NO: 148-149 - skipped (2 entries)

SEQ ID NO: 150 - protein, 6 aa, synthetic construct
NPNANP

SEQ ID NO: 151-152 - skipped (2 entries)

SEQ ID NO: 153 - protein, 6 aa, synthetic construct
NANPNA

SEQ ID NO: 154 - skipped

SEQ ID NO: 155 - DNA, 432 bp, unidentified
  source 1..432: mol_type = other DNA; note = Plasmodium falciparum
aatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcct

SEQ ID NO: 156 - protein, 144 aa, unidentified
  source 1..144: note = Plasmodium falciparum
NANPNANPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANP

SEQ ID NO: 157 - RNA, 432 nt, unidentified
  source 1..432: mol_type = other RNA; note = Plasmodium falciparum
aatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcct

SEQ ID NO: 158 - DNA, 81 bp, Human alphaherpesvirus 1
  source 1..81: mol_type = other DNA
atggggggggctgccgccaggttgggggccgtgattttgtttgtcgtcatagtgggcctccatggggtccgcagcaaatat

SEQ ID NO: 159 - protein, 27 aa, Human alphaherpesvirus 1
MGGAAARLGAVILFVVIVGLHGVRSKY

SEQ ID NO: 160 - RNA, 81 nt, unidentified
  source 1..81: mol_type = other RNA; note = Human alphaherpesvirus 1
atggggggggctgccgccaggttgggggccgtgattttgtttgtcgtcatagtgggcctccatggggtccgcagcaaatat

SEQ ID NO: 161 - DNA, 81 bp, synthetic construct
  source 1..81: mol_type = other DNA
atgggaggagccgccgccagactgggagccgtgatcctgttcgtggtgatcgtgggactgcatggagtgagaagcaagtac

SEQ ID NO: 162 - protein, 27 aa, Human alphaherpesvirus 1
MGGAAARLGAVILFVVIVGLHGVRSKY

SEQ ID NO: 163 - RNA, 81 nt, Human alphaherpesvirus 1
  source 1..81: mol_type = other RNA
atgggaggagccgccgccagactgggagccgtgatcctgttcgtggtgatcgtgggactgcatggagtgagaagcaagtac

SEQ ID NO: 164 - DNA, 81 bp, Human alphaherpesvirus 1
  source 1..81: mol_type = other DNA
atggggggggctgccgccaggttgggggccgtgattttgtttgtcgtcatagtgggcctccatggggtccgcggcaaatat

SEQ ID NO: 165 - protein, 27 aa, Human alphaherpesvirus 1
MGGAAARLGAVILFVVIVGLHGVRGKY

SEQ ID NO: 166 - RNA, 81 nt, Human alphaherpesvirus 1
  source 1..81: mol_type = other RNA
atggggggggctgccgccaggttgggggccgtgattttgtttgtcgtcatagtgggcctccatggggtccgcggcaaatat

SEQ ID NO: 167 - DNA, 75 bp, Human alphaherpesvirus 2
  source 1..75: mol_type = other DNA
atggggcgtttgacctccggcgtcgggacggcggccctgctagttgtcgcggtgggactccgcgtcgtctgcgcc

SEQ ID NO: 168 - protein, 25 aa, Human alphaherpesvirus 2
MGRLTSGVGTAALLVVAVGLRVVCA

SEQ ID NO: 169 - RNA, 75 nt, Human alphaherpesvirus 2
  source 1..75: mol_type = other RNA
atggggcgtttgacctccggcgtcgggacggcggccctgctagttgtcgcggtgggactccgcgtcgtctgcgcc

SEQ ID NO: 170 - DNA, 84 bp, Human alphaherpesvirus 2
  source 1..84: mol_type = other DNA
atggggcgtttgacctccggcgtcgggacggcggccctgctagttgtcgcggtgggactccgcgtcgtctgcgccaaatacgcc

SEQ ID NO: 171 - protein, 28 aa, Human alphaherpesvirus 2
MGRLTSGVGTAALLVVAVGLRVVCAKYA

SEQ ID NO: 172 - RNA, 84 nt, Human alphaherpesvirus 2
  source 1..84: mol_type = other RNA
atggggcgtttgacctccggcgtcgggacggcggccctgctagttgtcgcggtgggactccgcgtcgtctgcgccaaatacgcc

SEQ ID NO: 173 - DNA, 54 bp, unidentified
  source 1..54: mol_type = other DNA; note = Plasmodium falciparum
atgatgagaaaattagctattttatctgtttcttcctttttatttgttgaggcc

SEQ ID NO: 174 - protein, 18 aa, unidentified
  source 1..18: note = Plasmodium falciparum
MMRKLAILSVSSFLFVEA

SEQ ID NO: 175 - RNA, 54 nt, unidentified
  source 1..54: mol_type = other RNA; note = Plasmodium falciparum
atgatgagaaaattagctattttatctgtttcttcctttttatttgttgaggcc

SEQ ID NO: 176 - DNA, 96 bp, Zaire ebolavirus
  source 1..96: mol_type = other DNA
atgggtgttacaggaatcttgcagctgcctagagatcgattcaagaggacatcattctttctgtgggtgattatcctgttccaaagaacattttcc

SEQ ID NO: 177 - protein, 32 aa, Zaire ebolavirus
MGVTGILQLPRDRFKRTSFFLWVIILFQRTFS

SEQ ID NO: 178 - RNA, 96 nt, Zaire ebolavirus
  source 1..96: mol_type = other RNA
atgggtgttacaggaatcttgcagctgcctagagatcgattcaagaggacatcattctttctgtgggtgattatcctgttccaaagaacattttcc

SEQ ID NO: 179 - DNA, 57 bp, Severe acute respiratory syndrome-related coronavirus
  source 1..57: mol_type = other DNA
atgtttgtgtttcttgtgctgctgcctcttgtgtcttctcagtgtgtgaatttgaca

SEQ ID NO: 180 - protein, 19 aa, Severe acute respiratory syndrome-related coronavirus
MFVFLVLLPLVSSQCVNLT

SEQ ID NO: 181 - RNA, 56 nt, Severe acute respiratory syndrome-related coronavirus
  source 1..56: mol_type = other RNA
tgtttgtgtttcttgtgctgctgcctcttgtgtcttctcagtgtgtgaatttgaca

SEQ ID NO: 182 - DNA, 63 bp, Homo sapiens
  source 1..63: mol_type = other DNA
atggattggatttggagaatcctgttcctcgtgggagccgctacaggagcccactcccagatg

SEQ ID NO: 183 - protein, 21 aa, Homo sapiens
MDWIWRILFLVGAATGAHSQM

SEQ ID NO: 184 - RNA, 63 nt, Homo sapiens
  source 1..63: mol_type = other RNA
atggattggatttggagaatcctgttcctcgtgggagccgctacaggagcccactcccagatg

SEQ ID NO: 185 - skipped

SEQ ID NO: 186 - protein, 20 aa, Homo sapiens
METPAQLLFLLLLWLPDTTG

SEQ ID NO: 187-188 - skipped (2 entries)

SEQ ID NO: 189 - protein, 18 aa, synthetic construct
MDWTWILFLVAAATRVHS

SEQ ID NO: 190-191 - skipped (2 entries)

SEQ ID NO: 192 - protein, 24 aa, Japanese encephalitis virus
MLGSNSGQRVVFTILLLLVAPAYS

SEQ ID NO: 193-194 - skipped (2 entries)

SEQ ID NO: 195 - protein, 17 aa, unidentified
  source 1..17: note = Vesicular stomatitis virus
MKCLLYLAFLFIGVNCA

SEQ ID NO: 196-197 - skipped (2 entries)

SEQ ID NO: 198 - protein, 24 aa, Anopheles gambiae
MCRGLSAVLILLVSLSAQLHVVVG

SEQ ID NO: 199 - skipped

SEQ ID NO: 200 - DNA, 57 bp, Homo sapiens
  source 1..57: mol_type = other DNA
atggagttgggactgagctggattttccttttggctattttaaaaggtgtccagtgt

SEQ ID NO: 201 - protein, 19 aa, Homo sapiens
MELGLSWIFLLAILKGVQC

SEQ ID NO: 202 - RNA, 57 nt, Homo sapiens
  source 1..57: mol_type = other RNA
atggagttgggactgagctggattttccttttggctattttaaaaggtgtccagtgt

SEQ ID NO: 203 - DNA, 57 bp, Homo sapiens
  source 1..57: mol_type = other DNA
atggaactggggctccgctgggttttccttgttgctattttagaaggtgtccagtgt

SEQ ID NO: 204 - protein, 19 aa, Homo sapiens
MELGLRWVFLVAILEGVQC

SEQ ID NO: 205 - RNA, 57 nt, Homo sapiens
  source 1..57: mol_type = other RNA
atggaactggggctccgctgggttttccttgttgctattttagaaggtgtccagtgt

SEQ ID NO: 206 - DNA, 57 bp, Homo sapiens
  source 1..57: mol_type = other DNA
atgaaacacctgtggttcttcctcctgctggtggcagctcccagatgggtcctgtcc

SEQ ID NO: 207 - protein, 19 aa, Homo sapiens
MKHLWFFLLLVAAPRWVLS

SEQ ID NO: 208 - RNA, 57 nt, Homo sapiens
  source 1..57: mol_type = other RNA
atgaaacacctgtggttcttcctcctgctggtggcagctcccagatgggtcctgtcc

SEQ ID NO: 209 - DNA, 57 bp, Homo sapiens
  source 1..57: mol_type = other DNA
atggactggacctggaggatcctcttcttggtggcagcagcaacaggtgcccactcg

SEQ ID NO: 210 - protein, 19 aa, Homo sapiens
MDWTWRILFLVAAATGAHS

SEQ ID NO: 211 - RNA, 57 nt, Homo sapiens
  source 1..57: mol_type = other RNA
atggactggacctggaggatcctcttcttggtggcagcagcaacaggtgcccactcg

SEQ ID NO: 212 - DNA, 57 bp, Homo sapiens
  source 1..57: mol_type = other DNA
atggactggacctggaggttcctctttgtggtggcagcagctacaggtgtccagtcc

SEQ ID NO: 213 - protein, 19 aa, Homo sapiens
MDWTWRFLFVVAAATGVQS

SEQ ID NO: 214 - RNA, 57 nt, Homo sapiens
  source 1..57: mol_type = other RNA
atggactggacctggaggttcctctttgtggtggcagcagctacaggtgtccagtcc

SEQ ID NO: 215 - DNA, 57 bp, Homo sapiens
  source 1..57: mol_type = other DNA
atggagtttgggctgagctggctttttcttgtggcgattctaaaaggtgtccagtgt

SEQ ID NO: 216 - protein, 19 aa, Homo sapiens
MEFGLSWLFLVAILKGVQC

SEQ ID NO: 217 - RNA, 57 nt, Homo sapiens
  source 1..57: mol_type = other RNA
atggagtttgggctgagctggctttttcttgtggcgattctaaaaggtgtccagtgt

SEQ ID NO: 218 - DNA, 57 bp, Homo sapiens
  source 1..57: mol_type = other DNA
atggagtttgggctgagctgggttttcctcgttgctctttttagaggtgtccagtgt

SEQ ID NO: 219 - protein, 19 aa, Homo sapiens
MEFGLSWVFLVALFRGVQC

SEQ ID NO: 220 - RNA, 57 nt, Homo sapiens
  source 1..57: mol_type = other RNA
atggagtttgggctgagctgggttttcctcgttgctctttttagaggtgtccagtgt

SEQ ID NO: 221 - DNA, 78 bp, Homo sapiens
  source 1..78: mol_type = other DNA
atggacctcctgcacaagaacatgaaacacctgtggttcttcctcctcctggtggcagctcccagatgggtgctgtcc

SEQ ID NO: 222 - protein, 26 aa, Homo sapiens
MDLLHKNMKHLWFFLLLVAAPRWVLS

SEQ ID NO: 223 - RNA, 78 nt, Homo sapiens
  source 1..78: mol_type = other RNA
atggacctcctgcacaagaacatgaaacacctgtggttcttcctcctcctggtggcagctcccagatgggtgctgtcc

SEQ ID NO: 224 - DNA, 66 bp, Homo sapiens
  source 1..66: mol_type = other DNA
atggacatgagggtccctgctcagctcctggggctcctgctgctctggctctcaggtgccagatgt

SEQ ID NO: 225 - protein, 22 aa, Homo sapiens
MDMRVPAQLLGLLLLWLSGARC

SEQ ID NO: 226 - RNA, 66 nt, Homo sapiens
  source 1..66: mol_type = other RNA
atggacatgagggtccctgctcagctcctggggctcctgctgctctggctctcaggtgccagatgt

SEQ ID NO: 227 - DNA, 66 bp, Homo sapiens
  source 1..66: mol_type = other DNA
atgaaatacctattgcctacggcagccgctggattgttattactcgcggcccagccggccatggcc

SEQ ID NO: 228 - protein, 22 aa, Homo sapiens
MKYLLPTAAAGLLLLAAQPAMA

SEQ ID NO: 229 - RNA, 66 nt, Homo sapiens
  source 1..66: mol_type = other RNA
atgaaatacctattgcctacggcagccgctggattgttattactcgcggcccagccggccatggcc

SEQ ID NO: 230 - skipped

SEQ ID NO: 231 - protein, 20 aa, unidentified
  source 1..20: note = Plasmodium falciparum
FNVVNSSIGLIMVLSFLFLN

SEQ ID NO: 232 - RNA, 60 nt, unidentified
  source 1..60: mol_type = other RNA; note = Plasmodium falciparum
ttcaacgtggtcaacagcagcatcggcctgattatggtgctgtccttcctgttcctgaac

SEQ ID NO: 233 - DNA, 123 bp, Human alphaherpesvirus 1
  source 1..123: mol_type = other DNA
ggcctgatcgccggcgcggtgggcggcagtctcctggcagccctggtcatttgcggaattgtgtactggatgcgccgccacactcaaaaagccccaaagcgcatacgcctcccccacatccgg

SEQ ID NO: 234 - protein, 41 aa, Human alphaherpesvirus 1
GLIAGAVGGSLLAALVICGIVYWMRRHTQKAPKRIRLPHIR

SEQ ID NO: 235 - RNA, 123 nt, Human alphaherpesvirus 1
  source 1..123: mol_type = other RNA
ggcctgatcgccggcgcggtgggcggcagtctcctggcagccctggtcatttgcggaattgtgtactggatgcgccgccacactcaaaaagccccaaagcgcatacgcctcccccacatccgg

SEQ ID NO: 236 - DNA, 111 bp, synthetic construct
  source 1..111: mol_type = other DNA
cctaacaaaggcagcggcacaacctccggcaccacaagactgctgtctggccacacctgtttcaccctgactggcctgctgggcaccctggttacaatgggactgctgaca

SEQ ID NO: 237 - protein, 37 aa, Homo sapiens
PNKGSGTTSGTTRLLSGHTCFTLTGLLGTLVTMGLLT

SEQ ID NO: 238 - RNA, 111 nt, synthetic construct
  source 1..111: mol_type = other RNA
cctaacaaaggcagcggcacaacctccggcaccacaagactgctgtctggccacacctgtttcaccctgactggcctgctgggcaccctggttacaatgggactgctgaca

SEQ ID NO: 239 - DNA, 60 bp, unidentified
  source 1..60: mol_type = other DNA; note = Plasmodium falciparum
gaactcgatggctccaaaaatgagtggggatggtcaaaatccaagctcggggcaaatgcc

SEQ ID NO: 240 - protein, 20 aa, unidentified
  source 1..20: note = Plasmodium falciparum
ELDGSKNEWGWSKSKLGANA

SEQ ID NO: 241 - RNA, 60 nt, unidentified
  source 1..60: mol_type = other RNA; note = Plasmodium falciparum
gaactcgatggctccaaaaatgagtggggatggtcaaaatccaagctcggggcaaatgcc

SEQ ID NO: 242 - DNA, 504 bp, unidentified
  source 1..504: mol_type = other DNA; note = Plasmodium falciparum
gagaatgtgcaagtgtccgacgagctgttcaacgagctgctgaacagcgtggacgtgaacggcgaagtgaaagagaacatcctggaagaaagccaagtgaacgacgacatcttcaacagcctggtcaagagcgtgcagcaagagcagcagcacaacgtggaagaaaaggtggaagaatccgtcgaggaaaacgacgaggaaagcgttgaagaaaacgttgaggaaaatgtcgaagaaaacgatgacgagagcgtggccagcagcgtcgaagaatctatcgccagctccgtggacgagagcatcgacagcagcattgaggaaaacgtggcccctacagtggaagaaatcgtggccccaactgtcgaggaaattgtggctcccagcgtggtggaatctgtggcccctagcgttgaggaatcagttgaggaaaatgttgaggaaagcgtggccgagaacgtggaagaatctgtggccgctgagaatgtcgaggaatccgtcgaaaac

SEQ ID NO: 243 - protein, 190 aa, unidentified
  source 1..190: note = Plasmodium falciparum
ENVQVSDELFNELLNSVDVNGEVKENILEESQVNDDIFNSLVKSVQQEQQHNVEEKVEESVEENDEESVEENVEENVEENDDESVASSVEESIASSVDESIDSSIEENVAPTVEEIVAPTVEEIVAPSVVESVAPSVEESVEENVEESVAENVEESVAENVEESVAENVEESVAENVEESVAENVEESVA

SEQ ID NO: 244 - RNA, 504 nt, unidentified
  source 1..504: mol_type = other RNA; note = Plasmodium falciparum
gagaatgtgcaagtgtccgacgagctgttcaacgagctgctgaacagcgtggacgtgaacggcgaagtgaaagagaacatcctggaagaaagccaagtgaacgacgacatcttcaacagcctggtcaagagcgtgcagcaagagcagcagcacaacgtggaagaaaaggtggaagaatccgtcgaggaaaacgacgaggaaagcgttgaagaaaacgttgaggaaaatgtcgaagaaaacgatgacgagagcgtggccagcagcgtcgaagaatctatcgccagctccgtggacgagagcatcgacagcagcattgaggaaaacgtggcccctacagtggaagaaatcgtggccccaactgtcgaggaaattgtggctcccagcgtggtggaatctgtggcccctagcgttgaggaatcagttgaggaaaatgttgaggaaagcgtggccgagaacgtggaagaatctgtggccgctgagaatgtcgaggaatccgtcgaaaac

SEQ ID NO: 245 - protein, 391 aa, Anopheles gambiae
MCRGLSAVLILLVSLSAQLHVVVGEEAPKPEKEICGLKVGRLLDSVKGWLSVSQQEKCPLNKYCENKIQADQYNLVPLTCIRWRSLNPASPTGSLGGKDVVSKIDAAMSNFKTLFEPMKADLAKLEEEVKRQVLDAWKALEPLQKEVYRSTLASGRIERAVFYSFMEMGDNVKLDNYFQPANVEELLKYAWALPMHKKQRSMYDLIGQLVQSSKSPMLQTLHAVELATVVNPELENRENLLNDQVVQLRDNLYKNSFATLVSIARHFPDHFDTLRQRLFKLPDGSKPGADTLPNIVNFIAQLPSDELRLSSVDLLLQSLTAENGTLVQDPEYVYRLSQLAHAMPSLVDVKAHPDLQQSVDDLMAKFNTPIDGKTLQYFQNIGISPSSSVAT

SEQ ID NO: 246 - protein, 391 aa, Anopheles gambiae; identical to SEQ ID NO: 245

SEQ ID NO: 247-248 - skipped (2 entries)

SEQ ID NO: 249 - protein, 791 aa, unidentified
  source 1..791: note = Plasmodium falciparum
MVDFNDLSVELKKTELIKEDLRNLSHIINNEFSYFCQNENKNVSFNNNISSYYNDDIFSKSTLNNLYTSWKLEDFSHFDFSSILDILKRNQYVMCSIYFLLIFSCIYFLTLLLYTKCIKTTLKKWFCRYCSENINENNSNHNEQRTVLQNVINKSCYFITYSSIICLLLFLLLSGITYMHYFIKTKKGIHSNICNIYTRLDKFLLNKCLDPKKVDTSCYSAEHILNDLSSILEEYKKVKQQAKDDTLLDENTPFPLLERYITTFNKLNVLKNNINKNNTTLENEYFHTYPALKGISETLTTIISEGNKNFGNARNVIKEVKSTIKYSFHTVDETIRNVFKDSVPKITGLITQAGKSIKGINNKYKIKERIPKYTNIILLTNIILLLPPFLILLGIIIFMIFILMGYIQKNNNFFIKLFGHFSAYFGLLTIIILSFGILFLSTSVIGGTSCILSERILKNELRFDILNNTLIDYCIKNESAPLIDDDITTSFVAKINSFDTGHIDHNINEYEKHFTILKESFFHKSLKFMDYIWIVIMKRENNTFLNRIRTEQVKKSLLITGIINENIKYENMEAIGIRSYLTTLNKIIFPENNGKICFNDIICEKENNTYNITENSKTTDQKYRNIRDGMDEHLRNDLDAIVQLFVYKARILKENIFDINDLDSNEKNKIGWSEYTPRNINGTQKKSIINTFLVNVIESINFSEIINFFDKMRDQFNVLKDLILLKIDTLTENTKCNKLVKELINVRKDYCNNVVLNLSTLSVYLIIFSITSFLLWYLFLFLWFYYNIKPS

SEQ ID NO: 250 - skipped

SEQ ID NO: 251 - DNA, 498 bp, unidentified
  source 1..498: mol_type = other DNA; note = Plasmodium falciparum
gagaatgtgcaagtgtccgacgagctgttcaacgagctgctgaacagcgtggacgtgaacggcgaagtgaaagagaacatcctggaagaaagccaagtgaacgacgacatcttcaacagcctggtcaagagcgtgcagcaagagcagcagcacaacgtggaagaaaaggtggaagaatccgtcgaggaaaacgacgaggaaagcgttgaagaaaacgttgaggaaaatgtcgaagaaaacgatgacgagagcgtggccagcagcgtcgaagaatctatcgccagctccgtggacgagagcatcgacagcagcattgaggaaaacgtggcccctacagtggaagaaatcgtggccccaactgtcgaggaaattgtggctcccagcgtggtggaatctgtggcccctagcgttgaggaatcagttgaggaaaatgttgaggaaagcgtggccgagaacgtggaagaatctgtggccgctgagaatgtcgaggaatccgtc

SEQ ID NO: 252 - protein, 190 aa, unidentified; identical to SEQ ID NO: 243
  source 1..190: note = Plasmodium falciparum

SEQ ID NO: 253 - RNA, 498 nt, unidentified
  source 1..498: mol_type = other RNA; note = Plasmodium falciparum
gagaatgtgcaagtgtccgacgagctgttcaacgagctgctgaacagcgtggacgtgaacggcgaagtgaaagagaacatcctggaagaaagccaagtgaacgacgacatcttcaacagcctggtcaagagcgtgcagcaagagcagcagcacaacgtggaagaaaaggtggaagaatccgtcgaggaaaacgacgaggaaagcgttgaagaaaacgttgaggaaaatgtcgaagaaaacgatgacgagagcgtggccagcagcgtcgaagaatctatcgccagctccgtggacgagagcatcgacagcagcattgaggaaaacgtggcccctacagtggaagaaatcgtggccccaactgtcgaggaaattgtggctcccagcgtggtggaatctgtggcccctagcgttgaggaatcagttgaggaaaatgttgaggaaagcgtggccgagaacgtggaagaatctgtggccgctgagaatgtcgaggaatccgtc

SEQ ID NO: 254 - DNA, 117 bp, synthetic construct
  source 1..117: mol_type = other DNA
ggctatatccctgaggctcctagagatggccaggcctacgtcagaaaggatggcgaatgggtgctgctgagcaccttcctgggcagatctctggaagtgctgtttcaaggccctggc

SEQ ID NO: 255 - protein, 39 aa, synthetic construct
GYIPEAPRDGQAYVRKDGEWVLLSTFLGRSLEVLFQGPG

SEQ ID NO: 256 - RNA, 117 nt, synthetic construct
  source 1..117: mol_type = other RNA
ggctatatccctgaggctcctagagatggccaggcctacgtcagaaaggatggcgaatgggtgctgctgagcaccttcctgggcagatctctggaagtgctgtttcaaggccctggc

SEQ ID NO: 257 - skipped

SEQ ID NO: 258 - protein, 10 aa, synthetic construct
GGSGGGGSGG

SEQ ID NO: 259-260 - skipped (2 entries)

SEQ ID NO: 261 - protein, 11 aa, synthetic construct
SGGGGSGGGGS

SEQ ID NO: 262-263 - skipped (2 entries)

SEQ ID NO: 264 - protein, 6 aa, synthetic construct
GSGSGS

SEQ ID NO: 265-266 - skipped (2 entries)

SEQ ID NO: 267 - protein, 9 aa, synthetic construct
GSPGSGSGS

SEQ ID NO: 268-269 - skipped (2 entries)

SEQ ID NO: 270 - protein, 15 aa, synthetic construct
GGGGSGGGGSGGGGS

SEQ ID NO: 271-272 - skipped (2 entries)

SEQ ID NO: 273 - protein, 10 aa, synthetic construct
GGSLGGGGSG

SEQ ID NO: 274-278 - skipped (5 entries)

SEQ ID NO: 279 - protein, 4 aa, synthetic construct
GGGS

SEQ ID NO: 280-281 - skipped (2 entries)

SEQ ID NO: 282 - protein, 10 aa, synthetic construct
AGNRVRRSVG

SEQ ID NO: 283 - skipped

SEQ ID NO: 284 - DNA, 1197 bp, unidentified; identical to SEQ ID NO: 32
  source 1..1197: mol_type = other DNA; note = Plasmodium falciparum

SEQ ID NO: 285 - RNA, 1197 nt, unidentified; identical to SEQ ID NO: 34
  source 1..1197: mol_type = other RNA; note = Plasmodium falciparum

SEQ ID NO: 286 - skipped

SEQ ID NO: 287 - protein, 574 aa, unidentified
  source 1..574: note = Plasmodium falciparum
MNHLGNVKYLVIVFLIFFDLFLVNGRDVQNNIVDEIKYREEVCNDEVDLYLLMDCSGSIRRHNWVNHAVPLAMKLIQQLNLNDNAIHLYASVFSNNAREIIRLHSDASKNKEKALIIIKSLLSTNLPYGKTNLTDALLQVRKHLNDRINRENANQLVVILTDGIPDSIQDSLKESRKLSDRGVKIAVFGIGQGINVAFNRFLVGCHPSDGKCNLYADSAWENVKNVIGPFMKAVCVEVEKTASCGVWDEWSPCSVTCGKGTRSRKREILHEGCTSELQEQCEEERCLPKREPLDVPDEPEDDQPRPRGDNFAVEKPNENIIDNNPQEPSPNPEEGKGENPNGFDLDENPENPPNPPNPPNPPNPPNPPNPDIPEQEPNIPEDSEKEVPSDVPKNPEDDREENFDIPKKPENKHDNQNNLPNDKSDRYIPYSPLSPKVLDNERKQSDPQSQDNNGNRHVPNSEDRETRPHGRNNENRSYNRKHNNTPKHPEREEHEKPDNNKKKAGSDNKYKIAGGIAGGLALLACAGLAYKFVVPGAATPYAGEPAPFDETLGEEDKDLDEPEQFRLPEENEWN

SEQ ID NO: 288-289 - skipped (2 entries)

SEQ ID NO: 290 - protein, 1162 aa, unidentified
  source 1..1162: note = Plasmodium falciparum
MKHILYISFYFILVNLLIFHINGKIIKNSEKDEIIKSNLRSGSSNSRNRINEEKHEKKHVLSHNSYEKTKNNENNKFFDKDKELTMSNVKNVSQTNFKSLLRNLGVSENIFLKENKLNKEGKLIEHIINDDDDKKKYIKGQDENRQEDLEQERLAKEKLQGQQSDLEQERLAKEKLQEQQSDLEQERLAKEKLQEQQSDLEQDRLAKEKLQEQQSDLEQERRAKEKLQEQQSDLERTKTSKEKLHEQQSDLEQERRAKEKLQEQQSDLEQERLAKEKLQEQQSDLEQERLAKEKLQEQQSDLEQERLAKEKLQEQQSDLEQERLAKEKLQEQQSDLEQERLAKEKLQEQQSDLEQDRLAKEKLQEQQSDLEQERLAKEKLQEQQSDLEQERRAKEKLQEQQSDLEQERRAKEKLQEQQSDLEQERRAKEKLQEQQSDLEQERRAKEKLQEQQSDLEQERLAKEKLQEQQSDLEQERRAKEKLQEQQSDLEQERLAKEKLQEQQSDLEQERRAKEKLQEQQSDLEQERRAKEKLQEQQSDLEQDRLAKEKLQEQQSDLEQERRAKEKLQEQQSDLERTKASTETLHEQQSDLEQERLAKEKLQEQQSDLEQERLAKEKLQEQQSDLERTKASTETLREQQSDLEQEKLAKEKLQGQQSDLEQERLAKEKLQGQQSDLEQERLAKEKLQGQQSDLEQERLAKEKLQGQQSDLEQERLAKEKLQERQSDLEQERLAKEKLQEQQSDLEQERLAKEKLQEQQSDLEQDRLAKEKLQEQQSDLEQERLAKEKLQGQHSDLERTKASKETLQEQQSDLEQERLAKEKLQEQQSDLEQERRAKEKLQEQQSDLEQERRAKEKLQEQQSDLEQERRAKEKLQEQQRDLEQRKADTKKNLERKKEHGDVLAEDLYGRLEIPAIELPSENERGYYIPHQSSLPQDNRGNSRDSKEISIIENTNRESITTNVEGRRDIHKGHLEEKKDGSIKPEQKEDKSADIQNHTLETVNISDVNDFQISKYEDEISAEYDDSLIDEEEDDEDLDEFKPIVQYDNFQDEENIGIYKELEDLIEKNENLDDLDEGIEKSSEELSEEKIKKGKKYEKTKDNNFKPNDKSLYDEHIKKYKNDKQVNKEKEKFIKSLFHIFDGDNEILQIVDELSEDITKYFMKL

SEQ ID NO: 291-292 - skipped (2 entries)

SEQ ID NO: 293 - protein, 165 aa, unidentified
  source 1..165: note = Plasmodium
ERRAKEKLQEQQRDLEQRKADTKKNLERKKEHGDVLAEDLYGRLEIPAIELPSENERGYYIPHQSSLPQDNRGNSRDSKEISIIENTNRESITTNVEGRRDIHKGHLEEKKDGSIKPEQKEDKSADIQNHTLETVNISDVNDFQISKYEDEISAEYDDSLIDEEE

SEQ ID NO: 294-295 - skipped (2 entries)

SEQ ID NO: 296 - protein, 132 aa, unidentified
  source 1..132: note = Plasmodium
DDEDLDEFKPIVQYDNFQDEENIGIYKELEDLIEKNENLDDLDEGIEKSSEELSEEKIKKGKKYEKTKDNNFKPNDKSLYDEHIKKYKNDKQVNKEKEKFIKSLFHIFDGDNEILQIVDELSEDITKYFMKL

SEQ ID NO: 297-298 - skipped (2 entries)

SEQ ID NO: 299 - protein, 1558 aa, unidentified
  source 1..1558: note = Plasmodium falciparum
MTNSNYKSNNKTYNENNNEQITTIFNRTNMNPIKKCHMREKINKYFFLIKILTCTILIWAVQYANNSDINKSWKKNTYVDKKLNKLFNRSLGESQVNGELASEEVKEKILDLLEEGNTLTESVDDNKNLEEAEDIKENILLSNIEEPKENIIDNLLNNIGQNSEKQESVSENVQVSDELFNELLNSVDVNGEVKENILEESQVNDDIFNSLVKSVQQEQQHNVEEKVEESVEENDEESVEENVEENVEENDDESVASSVEESIASSVDESIDSSIEENVAPTVEEIVAPTVEEIVAPSVVESVAPSVEESVEENVEESVAENVEESVAENVEESVAENVEESVAENVEESVAENVEESVAENVEEIVAPTVEESVAPTVEEIVAPSVEESVAPSVEEIVVPTVEESVAENVEEIVAPSVEEIVAPSVEEIVAPTVEESVAPTVEEIVAPSVEESVAPSVEEIVVPTVEESVAENVEESVAENVEEIVAPSVEEIVAPSVEEIVAPSVEEIVAPSVEEIVAPSVEEIVAPSVEEIVAPSVEEIVAPSVEEIVAPTVEEIVAPTVEEIVAPSVEEIVAPTVEESVAENVATNLSDNLLSNLLGGIETEEIKDSILNEIEEVKENVVTTILENVEETTAESVTTFSNILEEIQENTITNDTIEEKLEELHENVLSAALENTQSEEEKKEVIDVIEEVKEEVATTLIETVEQAEEESASTITEIFENLEENAVESNENVAENLEKLNETVFNTVLDKVEETVEISGESLENNEMDKAFFSEIFDNVKGIQENLLTGMFRSIETSIVIQSEEKVDLNENVVSSILDNIENMKEGLLNKLENISSTEGVQETVTEHVEQNVYVDVDVPAMKDQFLGILNEAGGLKEMFFNLEDVFKSESDVITVEEIKDEPVQKEVEKETVSIIEEMEENIVDVLEEEKEDLTDKMIDAVEESIEISSDSKEETESIKDKEKDVSLVVEEVQDNDMDESVEKVLELKNMEEELMKDAVEINDITSKLIEETQELNEVEADLIKDMEKLKELEKALSEDSKEIIDAKDDTLEKVIEEEHDITTTLDEVVELKDVEEDKIEKVSDLKDLEEDILKEVKEIKELESEILEDYKELKTIETDILEEKKEIEKDHFEKFEEEAEEIKDLEADILKEVSSLEVEEEKKLEEVHELKEEVEHIISGDAHIKGLEEDDLEEVDDLKGSILDMLKGDMELGDMDKESLEDVTAKLGERVESLKDVLSSALGMDEEQMKTRKKAQRPKLEEVLLKEEVKEEPKKKITKKKVRFDIKDKEPKDEIVEVEMKDEDIDEDIEEDVEEDIEEDKVEDIDEDIDEDIDEDIGEDKDEVIDLIVQKEKRIEKVKEKKKKLEKKVEEGVSGLKKHVDEVMKYVQKIDKEVDKEVSKALESKNDVTNVLKQNQDFFSKVKNFVKKYKVFAAPFISAVAAFASYVVGFFTFSLFSSCVTIASSTYLLSKVDKTINKNKERPFYSFVFDIFKNLKHYLQQMKEKFSKEKNNNVIEVTNKAEKKGNVQVTNKTEKTTKVDKNNKVPKKSRTQKSK

SEQ ID NO: 300-301 - skipped (2 entries)

SEQ ID NO: 302 - protein, 106 aa, unidentified
  source 1..106: note = Plasmodium falciparum
MKTIIIVTLFILILNTIIINPCTCVPNTLPIEYFDYETLKENVKIRDSNTKTQLIILSAVCASILLSLSSLWGIRIHYNHEMNKKEINNYELNSDLLKDADFYMVE

SEQ ID NO: 303-304 - skipped (2 entries)

SEQ ID NO: 305 - protein, 302 aa, unidentified
  source 1..302: note = Plasmodium falciparum
MWLCKRGLSVNDTTKCDVPCKDFYMLFLSNKKEKIKCGTFFGYIFLSKFMKLSISLLLLALIQNILLSNVSLISGSHLYKRNSRKFAEGYMKGSGSEKNVYLSNKNKEINMNQQSDNKMCDECDDMNQPGDVNKNDKTSNDQANSSDSDCEPLPFGLKPSDLNRKVTEEDLERMIIELPGKLERKDMYLIWHYSHSLLRDKFNKMKSSLWSICGKLAHEHKLPFKIKMKKWWKCCGHVTDELLIKEHDDYNSIYNYINNESSSREQFLIFLNMIKHSWTTFTMETFIKCKISLENNMRNVTN

SEQ ID NO: 306-307 - skipped (2 entries)

SEQ ID NO: 308 - protein, 3597 aa, unidentified
  source 1..3597: note = Plasmodium falciparum
MKLSVLYLVVSLYFFDRKIVCNKKNGKSKSNNHNYFNNTTKHIDKQVIYNLPENDNLFHVLKYLIHGENKNILYDVDESLYTKIENKEYITKEDLLRTLALIEKKNVDCNDNIKKVKIAKLLIDINDLLRTNYIILDDYKEINKNDLTKNGFLLKLQNSLNKRDMVNSKRVNQDIILRNQYLEKNYKLCSISSDMLIISTPRNLNISDTSIKEIKHPSSINIMNEIFCDNVISSNVSNISPLQLNKSIGLYKPLLDLDIKSRIDEYRKKIITYYQDSMNEIFYLIRDEFNEIKKKFYLYIHPSKMPEAKPNNDIFKKEDMEGNEIITYVSNKIENKENIKNKEEYILKSNNNNVIKISSKRTKKVINIPIKKKKLSNDIKENVNSSTKSDNNNIISNETVDACTKIESENGNKKKDDNVKNINRIYHIFLKKKNNEKKCDISKQTLYNNSFKENDVIKTFTHNDNILKKIIIIKDVSREKPTIKEIIHQQPLPVENSELYNISEHIQNKCNCCFYSIPLIHIYHPNGSMHCLSCSQYEKNVNRKCPIYEKYITFNYNDTCLLCVPSSYITNNCFCNLIHSKGLGEKTNDVPKKLNMMPHFSSIIDVYGNKNIDGYNNFENDNNLLVYSNNDEPILKKNHKERPNKKCSNRYKLKCPVGNGSSDMFSGQSISIFPIIGDDINRGHYENCPYYDEEDVMCYNTITNKKININNEDDNYEKILYDKDKIVYLNSSSFDTINIKIEGEDLNMNDRNEENDLLDKESIEYINEINEKEYYDSIEACKDYRYHTFEESIKNVNNRECYKNVYYSTVHKKEDNIDKKCSHDIESRNKRVNEYEDECENDNNNNNNNNDDDDGEERNYFLFDLRFIYKTGFFTKRKTIINSYLKTNKIDITYNKEKILYLFKNLFEKTFKDINYQIKESLVHMFPIVQNSEIQGFDIDFGKVKLHYATSINMFKYSVFDLINMKIYFISPDKTYILLEDIIREIREEKENSNNDGSRRKNKIINKYLHEMDITNIEWSTENSKTNTTNSESKTTTTRENTIVIKEKIGTHDEKKKKCDNTKNNRNNNDDNDDNDNDNDNNNDNNNDNNNNNNNNNNNNNNNNNNNNNNSNSNSCDNNNKRDNDSFEHVDIQKNDTITTNIDCNIIHNNTIDSSNKNRIKYNLNILKFLLKYKDLLSKEDINMISDKNNLVTINKNDKTNIGDEIIEITIRKNEKRNIENNCTYTNNSIINNIDNNISESININGGEGINAVIETVDKYLDKYFSVHNMPIYYMENMKISKNIIYVTSDVNSDLDMLKMSILGICNITNKSIDNFNFFLVRNEAELYDPNKCQKIPEHIKNVQDLYNYSKMLDMKIIIAPVYAEDIDIIPKSFDYLFKLYKDGDDEKGVTTDSRDNDCQQKGNVDTQERNKTENDTTRITLSNIKHNNIIKNNNKNNNNNNNNSHNNNHSYYNHELSLFNDAESNKPNDNTTTSNDKNYYFFNYDNKKSLILNDPENDENIEYLSEEISIYEDEHIYDEGNLYNSFVIYIDNPFKRINIENAPILKGYTLNLFINDIMNKLIHIPSTFINTQSYKKGMCVMSICYCFNYYSLNIYTNNIILLNFDKDVTLYEVIKEFKSRENEYLLIKNKMNEECQDVDNLSNIKLPHTIDLIDSKYIHLEFPLLYIDEHSNFFQDKITLMNVPENLTVYELCKSMKNILNESLISFNLSTINDIKIYTIRGRQWEMVLDNLLVFGLKINYEDIFTILIDNNLLTKNYYELLSNIIIDFRDNNMYIKKLDIYIDYIFQPYTIYNIPIYITPNNLKYILLQYTNTFLSNSLLDEFFFTYNKRHTTINVVPEVRNWEHVEDSDVYIYEENDINDKKKEQKRNISFIYVLNMLSTFYKIKFNSSKLLESVMGEIHQLCGKRFNENLWLDIKKSTKNEKDIEIYLENSSKKVLIKNVPLDLLVWKLINIIMRGSFNIPIEDKEKLYNLFMLVPKNKDKIKDKNVDYYFLSTPGYTVEEMLRVFEKKKKPKKKGKEKNSANDNNNNNNNDNNNDNYNDNYNNDNNINSNNNYGDMKQTYYKILLIKAYPQHLHGIQNNERFNEYLTFNLEALPSVIDVKNPTTCLSFYVNYKNENKMTHIINVPENISTDMLLKTILSDNFCRWKKLPNEEKMKFSLHLNKKEITNIKDYINNGDIAQLKTFILINNKKRRVQEKYIDLDNTTELGSNVNNISKEELKEIQLYDNVLREILNKNYIDYNNISLSGYTITVYVFDGNKYEPLTIYNVPLNITNKDIINFLLIKSNHINITKLIDEFLLLNEKLYNTYIVKNKNKNMLEEQNVLFIKKLNEEEEVFQLNESKFYLVHNPLFNPLDNLLQNIVHKKYDFKSEKIDIHSVGEKFEILTLNILINNDINNMHNNNVNNNYVNNNNINNNNINNNNINNNNINNNNINNNNFSFGKKKFVITVLNIPYNMYITEFLRYSIGYQRKWIYKFVLFYIIKGSEKYILNDGNDIIRHGKTVSEIFQICKSGDLCTLHIDINFNEADNNLENDIFNEEINAKEMKNVDVDSHADVDVDLDANVYLDANADVEGNINKFVNSLHNDNENRFIVLKELSKKIYTDDYENKKIDMSIICFEYNAGLYERNLFGSSRICNIPKNYTVGDVLRYVKNKIIEDTNISMLNDLQLKIIHNDNYIDIPKDMNIEYAINYLFIYTPFIVYPSDDILKYNNIIDLILYNTIIDIKENSFVKKDIPIYVKKGNIFKNVSLKYIPINIKEDELLTYLLHYLTDNHEVVSFIRDITCICVYPLCDSIYIFHEKKQLSLIPSISYHIQLKNVYYISTVETYENFYNILGHMELNLNKFLHIQNDGNNYKKKEAYNNIYYMNNVDELIHIDVNIIFPSIQKIIRVKNLNVNIKMEELLYKIFGCITNKIYKWGELFRNFVGKKEKINIVNNDIKNNSNNINSINNMNSINNMNNINNMNSINNINNINSINNINNCDNIDRIDNKVLKDYLCEEQNIITFIFCSHDNSIHDYLTSRMIYLPAIINYQTNYITFYSSINGFANNETTFHGFPNYLSPETMLTMLQYNFFHVIGKNYLKLYGSLYDKGTDEDIIYNTNDIRKSENKDIYNKNLIAMRGAIKFILRIKNKGKKKSLDTIQNLINNELNIACENVENDVDMEECNDFLSSLNNSSIFWKNGILGFMANSIVTRDNMNNSLILPYVDFEINEKMFLQNILYHINISPYLYKLFELTHIDKKCNTYKKKLKNSVAHIQCLLSYGFSIYFDLHYDSTFDKYKGFTLGDNSFDITQIHSFQNLHEQNFVDLILYNHDNTFITIKNVYRDMTVATFLNDLSKTRCHILGLTYNQISTYYKLTYEIYNETHDISPNLSIADVHNIVISFSKSSILLKIRKIDITNDISHNNNNNNNNDSDFIINSDNIDYINALPDLYLSPYPPVIDLSQNNFQVNIYMHSKFIPNDYSLRKDIVPHITINNVPSNVFILSLKELLKTIFIQHMEKSQTTTFQLNLYDFEVEFFILTFNRFTSKFHKMNSNVLSNLTISNFYSTYIHGGLFLNIDKIKIDIPNLLNDFIDHFSKVVIDFSHHNIVKQ

SEQ ID NO: 309-310 - skipped (2 entries)

SEQ ID NO: 311 - protein, 1964 aa, unidentified
  source 1..1964: note = Plasmodium falciparum
MEKGSILSFIFFCSVVIFIRFIGYFFCNRYMTEEPYNNIFEIIKPENLYSSLLLLSNEKENDFPSSTNCNGYMKCIPFYNNVSERWKRYNFIQLYIIRSALNIHVMSKYNMLNKYNKETNRLLKRNNNVENRINNISNHYLCSGFKKENRLFFLLFYKTIKMMKLYIRNLFMKYIKIYYKTKHFEKNIETNKKVVYVERDNLFDIERNNLFDILYMLKRIDSYVKNIYSIISNNFLYVIRIIFLPFEKIYFSLKSLIMIKKMNMSSSYYYYYVNMFSLYKKNYNKYEEIFIHEQRVIYPNEYLKNEMLDKYRRVIRILSGQHDNPFIDSLLINPEKIEKDDLDVKQKKKKIIEELKKKKENTNTNTNTSTNTSANTNTSTNTSANTNTSTKESHILDESKLETFYRDELDKMGKEEIETYFKGNIDKKSLDEFHKILLEELNKMDKDELYEMYREELNRIEQEKIRNMNKEEINKTYKDEINNMNSDQVDKIHREELEKIEKEKINKMDKDEIDKIYREELDKMDRDAIYSMYIEDISNKNIKDLIKNEKETNKDKNKKKDIDINKKKKKDIDIDVDIDKDIHKDHVEELYGEVKNKLSKEELDRMDRDALYRVYLEELDRMNRDELYRVYLEELEKIDKEEKEKIHREKLHKIEKEKINKMDKDQIDKIYEEELNKMDSDEIQHVRRAILQDIQKEKIQNLELEEIDRLYKEELDRMDREARYEIPMRNLSRNEKDNIIHRNIKNESNQKNKKENVNVFIIHDNNDSNNNNNNNNRDVNNLNNKHTNNNYNENVEVELVVRNLDKDKGAKIEDIIDYFNKEIKKDKNVNVSNIVNFLNSKVGKDNTPIQHKKENQVDVVRKNIQIIQEDNIKNKGQKDNTEMLDNNKEITNIDIKNVDDIKNVGDIKSVGDIKSVDDINNVDGIKNVDGIKNVDGIKNVDGINNVGDINNAGDTNNAGDINNVGDINNSVDIYNVEHIDEAEKKPNLDNPKKFDWTQVFKDKVTEKIKNEEKFNNSKENIQNDIRDKEIHKDDRIKGITSREKNAEEINNNEKKDKFVYEFYTSNKKENIDKEEENNIDDKNIKIEIEPNYEINNNFEEENKNEINVIIDKEAKNNMDKDDSNNNNNIQKNNIIIKDNTNVSEEVHITESSKEIAEFFNNIIKNSNILDMCSKMNASDSEKGFICINGNNYIINPGTYHIINIKYPDYNNVRKKWYDSMDCISINNKDENNNNKEHNYYNKNDDDLYLKKSVEEFIPGFLSNINKVDDLARIFTPSFIQNDIFLNCIYKYRNDFDKNNNIYSFPMKIFLRKNSTKIKGCSFQIDEDPLLYKDYSEKESFLSNKIILNNSNRNTECVLHASNEIVGFQCGPPYKSYDNIQYRHLTNKSNDIQKNIFGIYSNNNSSYSHLFKNIFNNEHKLYNVGGYFQTEPINCFEFVNDNINVEDILPGAVPFPRFDLIHHDLDVNQTRYILLNETNQDKTISCTCNYFTEPNIVYTGKIIIKVEEEKIYKTKKLQTEFNDIINNKKEIYHEKKMNHIIKEKKDEDENDMSFNKNYVNNYNENFKINDINNFYNRNNHPNNNYHNDYHNNHSSKGNHTNKIHDTFLKNKYNISFNNLKFYNIKHENKNNQDVINYEYNVDDYHEVQDGQDESFKEEEDFIDFKENIINDNTNHNNTDLDDDKYNKYHNNNNNNNNNSSFKSIESNLDLQRSILQSGDTQQVVVINKSKNVNVLFPDKKKNHTHPNEEKRTFPKYISLVYKEKNKDNEKIDKTLTFIKEFYPPLLRGGKDSDPENKQTGVEINNGVEKKNDVQIKNDVEIKNDVEIKNDVEINDDVEIKNDVEIKNDVEIKNDVEINDDVEINDDVEINNGVEINDGVENKDNIHEGNNNLENDSFNEDTIEEPFENIFDFINEETSSNENSEIILDSADSIKRKLGHNFLDIISAGKFKIRHKEKKTKNKKKK

SEQ ID NO: 312-313 - skipped (2 entries)

SEQ ID NO: 314 - protein, 162 aa, unidentified
  source 1..162: note = Plasmodium falciparum
MKILSVFFLALFFIIFNKESLAEKTNKGTGSGVSSKKKNKKGSGEPLIDVHDLISDMIKKEEELVEVNKRKSKYKLATSVLAGLLGVVSTVLLGGVGLVLYNTEKGRHPFKIGSSDPADNANPDADSESNGEPNAGPQVTAQDVTPEQPQGDDNNLVSGTEH

SEQ ID NO: 315-316 - skipped (2 entries)

SEQ ID NO: 317 - protein, 287 aa, unidentified
  source 1..287: note = Plasmodium falciparum
MKVSYIFSFFLLFFVYKNTNTVVCDNGYGDLAATSALTTVIKDPISLTIKDIYEHGVKNPFTKIIHKLKKFIRYRKVLRWSRMWWVLLVREIVGDNTIEKKTEKALREIWDQCTIAVYNNTLNAVESKPLLFLHGILNECRNNFATKLRQDPSLIVAKIDQIIKSQIYRFWVSEPYLKIGRSHTLYTHITPDAVPQLPKECTLKHLSSYMEEKLKSMESKKNIESGKYEFDVDSSETDSTKDDGKPDDDDDDDDNFDDDDNFDDDTVEEEDASGDLFKNEKKDENKE

SEQ ID NO: 318-319 - skipped (2 entries)

SEQ ID NO: 320 - protein, 151 aa, unidentified
  source 1..151: note = Plasmodium falciparum
MCSTYSVEPLVYDSYEYVYLKPKKAGTSAVYYPLNISWKYVAKKRSVGCFGSRKKYTLTPEAYYYPYYYYYVYYYPSAARLVRTTRKEKVLKENNNKESEDENKQDNVGTEKKECDCSEKEKYIPTYVPLTESYYFPPSALYVPHYSVLVP

SEQ ID NO: 321-322 - skipped (2 entries)

SEQ ID NO: 323 - protein, 863 aa, unidentified
  source 1..863: note = Plasmodium falciparum
MSSILLFFVVFQYLLISFSGTSYKRFVVDGASIFLRNPYKITLGKSEKKGKVFSEFSEEEDSIVRRDTEKKKKWFGKGNDVNKKVKEKKNVILEEKQIGKEENNLVGQQNDSVGQQNDSVGQQNDSVGQQNDPVGQQNDSVGQQNDPVGQQNDPVGQQNDYVGQQNDSVGQQNDYVGQQNDYVGQQNDPVGQQNDSVGQQNDYVGQQNDPVGQQNDPVGQQNDPVGQQNDSVGQQNDYVGQQNDPVGQQNDYVGQQNDYVGQQNDPVGQQNDPVGQQNDPVEQEDNVGIVKKEKKEKIIVEKENEIGVIERENISGVEVINKDSEEDIYNSNVDKNFPADDNNRDEILVRRKKVSNKDSLFMNDIIRGENEDNDDDNEEEEEKVSKNLENVKLGELSKLQYIFGTNITDNDDDSEIRSESEIDDIDELDEEEYDWTEHVYNYKPTTYLLPGLGGSTLIAEYKNATIHSCSRYLLNSKPFRIWISLSRLLSIQSNIYCTFDTIRLKYDEKKNIYYNQPGVFIDVEKFGNLKGIEYLDYFNNTGIGITKYFNVVGQYFTSHGYVDGESIIGAPYDWRYPLSQQNYKILKEHIEYIYEKRNGTKVNLIGHSLGGLYLNFFLSRVVSKKWKQKHLSKIIFISTPFKGSVKTIRALIQSRKDFISFRITKLIKLSIPESMMKALGNSLGSLFDILPYREYYKRDQVVILINMSNTPIDEDHVQYLVTLCGIYKPECYRNRADVNLKVYTLKNWHELLDDKLKAKYENYKLYRERYYNKDHGVPIYCLYSTINKKETEYLLYFETQNTREEPTIYYGTGDGTVGTESLQACSNFYNTVLTHHFSDTSHVGILYTNETAKYIYDIVESPN

SEQ ID NO: 324-325 - skipped (2 entries)

SEQ ID NO: 326 - protein, 969 aa, unidentified
  source 1..969: note = Plasmodium falciparum
MKIPFFILHILLLQFLLCLIRCYVHNDVIKFGEENSLKCSQGNLYVLHCEVQCLNGNNEIIHKRCNDDIEKKCNGNNKCIYFFEYELRKKTQSFRNKNSIEISECVESEQNEVKTSTTCLLSNSFILDEAFIQYFFFIKNKNEEPVICKDGNINIKSALLHSPFCEIKLKDISEYIRKKCDNNKECLIDPLDVQKNLLNEEDPCYINNSYVSVNVVCNKEEEIGDESTDSSSMEIQDSTSNEQDENVKGMSSSQEMNSNNDENKNQDNESDDDVNNNNNNNNDDQDEQGNDGDVTSSMNKNEDNKDLEHGSSNDVNNNTDTLVNNKENKEFVLKEKSSLTSKINKELAHRTALFNKLADNISLLLNKKYDSFEIKDVLEDRYNEMKRDANPDVYYIYLMDTLDIEKIEDINLEEVKMSLLASLKETMNKIDTIEKKIEEFKNKYISLYNKVKTTMPELFDLNEDLVLLYNDFPFDNGMISSDIFFKYNPSENIMDHQEMVKKGSITEDELRIVNDLEPLDNYRRRKRITELRKILVEKLRILYLEKNNLFNTQASCIKSYCYKNPLNLKTLEVLLKKNYYRLKENKDYDVVSSIIQHLDNVDANKKKKWLTHERILKKLQVLIAEGYKRINEKEKDIDRRMAVYNALYEKAQSYNLQKLFNDSNDFLKKYAIMGNSFDDGDEVFGSQSSNFNIFDSNNTDQNNEQEQPKQDDQLLNNNNDDVLSESNNENKEKTSDDATHKETQEKSDQEPSQNIQEDNSDEKHAENEENVEQIETDSNVSEEANDENKDNMQTTTDEGTEELQQNDEDAESLTKENSKSEEQENEDSTDAEAIDKEEVETEEKGKDEQKKDEQKEQDEEEDGEKENKHKSSETTNETVTDIEENKNEVKGEEHLQGSEQSIEASESSQKDETKETEDKEEYVNANDDESSEEDTTPNETNKTDNGSSFFFAMSNALLVILLLLFIEFL

SEQ ID NO: 327-328 - skipped (2 entries)

SEQ ID NO: 329 - protein, 362 aa, unidentified
  source 1..362: note = Plasmodium falciparum
MIIPFHKKFISFFQIVLVVLLLCRSINCDSRHVFIRTELSFIKNNVPCIRDMFFIYKRELYNICLDDLKGEEDETHIYVQKKVKDSWITLNDLFKETDLTGRPHIFAYVDVEEIIILLCEDEEFSNRKKDMTCHRFYSNDGKEYNNSEITISDYILKDKLLSSYVSLPLKIENREYFLICGVSPYKFKDDNKKDDILCMASHDKGETWGTKIVIKYDNYKLGVQYFFLRPYISKNDLSFHFYVGDNINNVKNVNFIECTHEKDLEFVCSNRDFLKDNKVLQDVSTLNDEYIVSYGNDNNFAECYIFFNNENSILIKPEKYGNTTAGCYGGTFVKIDENRTLFIYSSSQGIYNIHTIYYANYE

SEQ ID NO: 330-331 - skipped (2 entries)

SEQ ID NO: 332 - protein, 1086 aa, unidentified
  source 1..1086: note = Plasmodium falciparum
MFRIFFTLLIIILIKKTSAIDLIEGIFYEKNEIDKLTFSLDHRVRDNLKTDLILNNNGENDYAYLNKYVYTILNRDSTEKIKTFFSHNKDMKSCDYFISKEYNSSDKTNQICYKKTFCGVVIPNSEEIKTNKITNDKLYCAHFNSTHIIIYYISQPLLLEPHVVYEETFFEKGKNDQINCQGMYISLRSVHVHTHNAILQQETLTYIKNLCDGKNNCKFDFDSIKYENKSLTHYLFFINIQYQCISPLNLQENEMCDVYNDDTHKATCKYGFNKIELLKNVCEENYRCTQDICSVNQFCDGENETCTCKTSLLPSAKNNCEYNDLCTVLNCPENSTCEQIGNGKKAECKCENGKYYHNNKCYTKNDLELAIKIEPHKKEKFYKNNLYQGKALKPEYIFMQCENGFSIEVINAYVSCYRVSFNLNKLKYVTESLKKMCDGKTKCAYGNTIDPIDDLNHHNICNNFNTIFKYDYLCVFNNQNITSDKNSHLHSNIPSLYNSSILPDINKSKFHLISRNSRTNQYPHNNISMLEIQNEISSHNSNQFSTDPHTNSNNINNMNIKKVEIFRSRFSSKLQCQGGKINIDKAILKGGEGCNDLLLTNSLKSYCNDLSECDIGLIYHFDTYCINDQYLFVSYSCSNLCNKCHNNSTCYGNRFNYDCFCDNPYISKYGNKLCERPNDCESVLCSQNQVCQILPNDKLICQCEEGYKNVKGKCVPDNKCDLSCPSNKVCVIENGKQTCKCSERFVLENGVCICANDYKMEDGINCIAKNKCKRKEYENICTNPNEMCAYNEETDIVKCECKEHYYRSSRGECILNDYCKDINCKENEECSIVNFKPECVCKENLKKNNKGECIYENSCLINEGNCPKDSKCIYREYKPHECVCNKQGHVAVNGKCVLEDKCVHNKKCSENSICVNVMNKEPICVCTYNYYKKDGVCLIQNPCLKDNGGCSRNSECTFKYSKINCTCKENYKNKDDSCVPNTNEYDESFTFQYNDDASIILGACGMIEFSYIYNQIIWKINNSKESYVFYYDYPTAGNIEVQIKNEIFHTIIYLKKKIGNSVIYDDFQVDHQTCIYENVFYYSNQN

SEQ ID NO: 333-334 - skipped (2 entries)

SEQ ID NO: 335 - protein, 791 aa, unidentified; identical to SEQ ID NO: 249
  source 1..791: note = Plasmodium falciparum

SEQ ID NO: 336-337 - skipped (2 entries)

SEQ ID NO: 338 - protein, 622 aa, unidentified
  source 1..622: note = Plasmodium falciparum
MRKLYCVLLLSAFEFTYMINFGRGQNYWEHPYQNSDVYRPINEHREHPKEYEYPLHQEHTYQQEDSGEDENTLQHAYPIDHEGAEPAPQEQNLFSSIEIVERSNYMGNPWTEYMAKYDIEEVHGSGIRVDLGEDAEVAGTQYRLPSGKCPVFGKGIIIENSNTTFLTPVATGNQYLKDGGFAFPPTEPLMSPMTLDEMRHFYKDNKYVKNLDELTLCSRHAGNMIPDNDKNSNYKYPAVYDDKDKKCHILYIAAQENNGPRYCNKDESKRNSMFCFRPAKDISFQNYTYLSKNVVDNWEKVCPRKNLQNAKFGLWVDGNCEDIPHVNEFPAIDLFECNKLVFELSASDQPKQYEQHLTDYEKIKEGFKNKNASMIKSAFLPTGAFKADRYKSHGKGYNWGNYNTETQKCEIFNVKPTCLINNSSYIATTALSHPIEVENNFPCSLYKDEIMKEIERESKRIKLNDNDDEGNKKIIAPRIFISDDKDSLKCPCDPEMVSNSTCRFFVCKCVERRAEVTSNNEVVVKEEYKDEYADIPEHKPTYDKMKIIIASSAAVAVLATILMVYLYKRKGNAEKYDKMDEPQDYGKSNSRNDEMLDPEASFWGEEKRASHTTPVLMEKPYY

SEQ ID NO: 339-340 - skipped (2 entries)

SEQ ID NO: 341 - protein, 673 aa, unidentified
  source 1..673: note = Plasmodium falciparum
MNVLFLSYNICILFFVVCTLNFSTKCFSNGLLKNQNILNKSFDSITGRLLNETELEKNKDDNSKSETLLKEEKDEKDDVPTTSNDNLKNAHNNNEISSSTDPTNIINVNDKDNENSVDKKKDKKEKKHKKDKKEKKEKKDKKEKKDKKEKKHKKEKKHKKDKKKEENSEVMSLYKTGQHKPKNATEHGEENLYEEMVSEINNNAQGGLLLSSPYQYREQGGCGIISSVHETSNDTKDNDKENISEDKKEDHQQEEMLKTLDKKERKQKEKEMKEQEKIEKKKKKQEEKEKKKQEKERKKQEKKERKQKEKEMKKQKKIEKERKKKEEKEKKKKKHDKENEETMQQPDQTSEETNNEIMVPLPSPLTDVTTPEEHKEGEHKEEEHKEGEHKEGEHKEEEHKEEEHKKEEHKSKEHKSKGKKDKGKKDKGKHKKAKKEKVKKHVVKNVIEDEDKDGVEIINLEDKEACEEQHITVESRPLSQPQCKLIDEPEQLTLMDKSKVEEKNLSIQEQLIGTIGRVNVVPRRDNHKKKMAKIEEAELQKQKHVDKEEDKKEESKEVEEESKEVQEDEEEVEEDEEEEEEEEEEEEEEEEEEEEEEDEVEEDEDDAEEDEDDAEEDEDDAEEDDDDAEEDDDDAEEDDDEDEDEDEEEEEDEEEEEESEKKIKRNLRKNAKI

SEQ ID NO: 342-343 - skipped (2 entries)

SEQ ID NO: 344 - protein, 408 aa, unidentified
  source 1..408: note = Plasmodium falciparum
MLLFFAKLVVFTFFFWLLKYGKTRSYPKSGHKGHTKLNQPVVRTLADFNDMFANQKNTFNFLKHINHYKNEQDTNNTHTPNHDEYSHNLPKNHEESNANMNNHNSFNDKSVNKKEAFDQFLQTLLNNYEIMHKEDESKESNQHNYKEGPSYEDKKNMYKEILKGYYNVFFENYANDTESNVHNKPEEVHKHEEIHKHRKLHKHEEVHKPEEFHKPEEFHKHEKVHKHEEVHKPEEVHKHEENHKHEENHKPQMVGQAPPEKEIRQESRTLILGSFPQAGEILREDLWNKEDNKFSYALDPNDYASIEDKLLGSIFGYFKKNHDNLVKHLLQQINTYKHKYMELKEQYINEVMKLKKIYNKSIMVIFIASCISILGPVMLHMHQNNPEEFFATILSFSISLGLHNLLLT

SEQ ID NO: 345-346 - skipped (2 entries)

SEQ ID NO: 347 - protein, 2074 aa, unidentified
  source 1..2074: note = Plasmodium falciparum
MMENKYPNELFCYINRYNINEIIENGEEKYVNEYDEDKNMSINHMNENDGICEYEIPFLLDYVDDSNKEDSEKNSLKSYLDDGASTILSKPDELENYNKQNENEFDENNNNKNNKIDQLKEKINIIIIPNKGVINNFEEILSMANRNDKNIEKKLNDRFYQICCKSIADINTHNLNKIKDLKKKKNNKGSLNIEHIDYGDIFLTIHDTLKSNNKIKGNNKTNLLHDSSYEIKKKTRRGTNIYKNPFHHRGSYLTSYENQKDIIYLNNLNNIMMDKYSNCSDSRKKEYSHFNSQEFSYDKYSMKDRMFLKNLYMKQNRLRDKRGKYHKLGDYQNIENYRKTGEHSFDCMNMSDIMHSNKMSHVNIMDHMIYKDNNNMSKLVDTINSREKDVKNYDDNFESYNNFFKNNNDEQHICLEYDDTYNLKDTVKNIIVEEEQCGKGVACICDKNEDVDDLFVSKKTNYSSNKKREDYEKVFLEDNLHLKQTPSKRTKINIIPDYYDNNRSNKSYKENEEDALFEVCGSLKNDDILYKDNKLNVINEDNIKEEDDKESVVHLDNDEDKKEEMYKDVYPNVLSCEKETIRRNEKYNKSLNSTSSFEKIDNPSEINVESKEDTEYFDLLIKKYEDTKINVYDNESLLLDLSNELREEMAKGDSNKNVNKVEDNDNKKENICHDNIMEDICHNNNVEDMYRNNNVEDMYRNNNVEDMYRNNNVEDMYRNNNVEDVCHNNNVEDVCHNNNVEDVCHNNNVEDVYHNNNVEDMYHDNNIEDVCHNNNVEDVCHNNNVEDHVNYDNEELNKKMDEMKEEKEERNEDRGIYDELLENDMCDLYNLKMHDLHNLKSYDFGLSKDLLKKDIFIYSNNLKNDDMDDDDNNNMNDIAIGENVIYENDIHENNIDDNDMYNNYVNGNDLYINNMQDDAMDDIVYDEEEIKSFLDKLKSDISNQMNVKNGNVEVTGNGGNEEMSYINNDENLQAFDLLDNFHMDDYGNNYNDNEEDGDGDGDDDEQKKRKQKELHNVNGKLNLSDLNELNVDDINNNFYMSTPRKSIDERKDTECQTDFPLLDVSRNTNRTPRRKSVEVILVEKKLKKKKQKCMDKYTDANEDSNRRYPKRNRIKTLRYWIGERELTERNPYTGEIDVVGFSECKNLQDLSPHIIGPIEYKKIYLKNLNSNEHEENEDNNGDIIENNNGDVIENNNGDIIEDNNANEKNHNNLESEGKGIVYDDVNNLHVHTNSDNSAHSKKIKGAPSRFSNTNNGRKKRRRRKFINVVNYIKKKKKKKLIKSMDNMEVTDNFKNDMSDENKQSGDENKQSGDENKQSGDENKQSGDENKQTNNDIKQSDNDIKQSDDIYMNEDMNLFNDLNDNFDNNEYFINNGDKDSHAEEEMAIENIQSKSIEKDILNNEEQDNNNIFDIDNELIDMKDGNVDEMESDEKLKTFEKLESLKSTTHLNNTDNCDVNLSEQTNEINYDEEKKVNKKTNHEKMKKKKKKKKKKKKKKKKEKKQIDIMYKNLSRLNLNLLLPTKKKVKKSKNSFKKEEEKQKKKNKKVKKIKGINKGEKIKSNKKENKDNNNDSSTECVVEGEKGKDLHEFNKNGNLEDEQMDVDISMNISSINCESDNKNVSKEGEEEKKDIAENKEEVDKNKEEVYMDKHEMDLNNEEVYMDKNEMDLNNEEVYMDKHEMDLNNEEVYMDKHEMDLNNEEVYMDKHEMDLNKEEVYMDKHEMDLNNEEVDKENEYDENILSDNIIYNENNSFGNNKNSFFNNTSPLKTEIINEEENSLNEMKEDINEYVEMENKLDTEKIKDSEKIGGKIEVDNKMISPINRHNFYLTILEGMNKNFPRQWNKNNITLSKNQGQIYKGRKEKKRKRSYRNDEKLLDHSILNDINISDKMDERNELLESIKSNSTINNVLEIIKYDNRKKIKKNDTNKEIIKYDNFTSKYNNKSNDIQLNGGIYINKFKLSLDMPINKLAVSSNLGPPSSIGSTEIQPIQKNFNDFKMNINVYCIRMEPHEKYSSYSHKNNLVVYIDKGEKINIIINMSKTYEKGDFFYIPRFSNFQIINDSRCDCVLYVCPLI

SEQ ID NO: 348-349 - skipped (2 entries)

SEQ ID NO: 350 - protein, 182 aa, unidentified
  source 1..182: note = Plasmodium falciparum
MNALRRLPVICSFLVFLVFSNVLCFRGNNGHNSSSSLYNGSQFIEQLNNSFTSAFLESQSMNKIGDDLAETISNELVSVLQKNSPTFLESSFDIKSEVKKHAKSMLKELIKVGLPSFENLVAENVKPPKVDPATYGIIVPVLTSLFNKVETAVGAKVSDEIWNYNSPDVSESEESLSDDFFD

SEQ ID NO: 351-352 - skipped (2 entries)

SEQ ID NO: 353 - protein, 245 aa, unidentified
  source 1..245: note = Plasmodium falciparum
MKMKIPICFLIILVLLKCVLSYNLNNDLSKNNNFSLNTYVRKDDVEDDSKNEIVDNIQKMVDDFSDDIGFVKTSMREVLLDTEASLEEVSDHVVQNISKYSLTIEEKLNLFDGLLEEFIENNKGLISNLSKRQQKLKGDKIKKVCDLILKKLKKLENVNKLIKYKIILKYGNKDNKKEMIQTLKNEEGLSDDFKNNLSNYETEQNNDDIKEIELVNFISTNYDKFVVNLEDLNKELLKDLNMALS

SEQ ID NO: 354-355 - skipped (2 entries)

SEQ ID NO: 356 - protein, 842 aa, unidentified
  source 1..842: note = Plasmodium falciparum
MKLRILKKHYYVVFILLYLYDISCFKCIRLNNRSIYKNKYKNNVHIGTNENIRSIEKYSNVLCNSILCKNDKISSFINQRKNVDDDDESENDDMYESTTAGSSSETDNESDEEENDSSDNNNSDEEQIENSNNNNSDEEQNDSSSNDNNDEENEEQDDVMDNDQNDKKIKHSFNLANESKHTKEERVKEEKKLKIYDFINDKEKRLNFNGDQKDEDNEENDDKDENTLENRNIISKHTSVFPGLYFIGIGYNLLFGNPLGEADSLIDPGYRAQIYLMEWALSKEGIANDLSTLQPVNGWIRKENACSRVESITECSSISDYTKSLSAEAKVSGSYWGIASFSASTGYSSFLHEVTKRSKKTFLVKSNCVKYTIGLPPYIPWDKTTAYKNAVNELPAVFTGLDKESECPSDVYEENKTKSNCENVSLWMKFFDIYGTHIIYESQLGGKITKIINVSTSSIEQMKKNGVSVKAKIQAQFGFGSAGGSTDVNSSNSSANDEQSYDMNEQLIVIGGNPIKDVTKEENLFEWSKTVTNHPMPINIKLTPISDSFDSDDLKESYDKAIIYYSRLYGLSPHDTMQKDDKDIIKILTNADTVTKNSAPPINAQCPHGKVVMFGFSLKQNFWDNTNALKGYNIEVCEAGSNSCTSKQGSSNKYDTSYLYMECGDQPLPFSEQVISESTSTYNTVKCPNDYSILLGFGISSSSGRINSAEYVYSTPCIPGMKSCSLNMNNDNQKSYIYVLCVDTTIWSGVNNLSLVALDGAHGKVNRSKKYSDGELVGTCPLDGTVLTGFKVEFHTSSPYVQTPFEKCAKSLKACSVHGSGHAIGIQNFKSLFIYMLCKNNK

SEQ ID NO: 357-358 - skipped (2 entries)

SEQ ID NO: 359 - protein, 229 aa, unidentified
  source 1..229: note = Plasmodium
MKVSKLVLFAHIFFIINILCQYICLNASKVNKKGKIAEEKKRKNIKNIDKAIEEHNKRKKLIYYSLIASGAIASVAAILGLGYYGYKKSREDDLYYNKYLEYRNGEYNIKYQDGAIASTSEFYIEPEGINKINLNKPIIENKNNVDVSIKRYNNFVDIARLSIQKHFEHLSNDQKDSHVNNMEYMQKFVQGLQENRNISLSKYQENKAVMDLKYHLQKVYANYLSQEEN

SEQ ID NO: 360-361 - skipped (2 entries)

SEQ ID NO: 362 - protein, 108 aa, unidentified
  source 1..108: note = Plasmodium falciparum
MKVSRHTVLLNIILIVSLLGCVLTLNLFSANDDKRALKDIDRTLEKLLRKKQIIISTAAVALAITLGGLFGSLGYKSWKNKNKSKDKVNDGSDSEELDSSKEDKDKDE

SEQ ID NO: 363-364 - skipped (2 entries)

SEQ ID NO: 365 - protein, 526 aa, unidentified
  source 1..526: note = Plasmodium falciparum
MIRIKKKLILTIIYIHLFILNRLSFENAIKKTKNQENNLTLLPIKSTEEEKDDIKNGKDIKKEIDNDKENIKTNNAKDHSTYIKSYLNTNVNDGLKYLFIPSHNSFIKKYSVFNQINDGMLLNEKNDVKNNEDYKNVDYKNVNFLQYHFKELSNYNIANSIDILQEKEGHLDFVIIPHYTFLDYYKHLSYNSIYHKSSTYGKCIAVDAFIKKINETYDKVKSKCNDIKNDLIATIKKLEHPYDINNKNDDSYRYDISEEIDDKSEETDDETEEVEDSIQDTDSNHTPSNKKKNDLMNRTFKKMMDEYNTKKKKLIKCIKNHENDFNKICMDMKNYGTNLFEQLSCYNNNFCNTNGIRYHYDEYIHKLILSVKSKNLNKDLSDMTNILQQSELLLTNLNKKMGSYIYIDTIKFIHKEMKHIFNRIEYHTKIINDKTKIIQDKIKLNIWRTFQKDELLKRILDMSNEYSLFITSDHLRQMLYNTFYSKEKHLNNIFHHLIYVLQMKFNDVPIKMEYFQTYKKNKPLTQ

SEQ ID NO: 366-367 - skipped (2 entries)

SEQ ID NO: 368 - protein, 378 aa, unidentified
  source 1..378: note = Plasmodium
MARNFECKKINSDDMTSSKKYSKNVGEKFNLISCTKLFALSMLFLICQNYENSPQSTSSHQEYQYNGLVLGNRILSELDQAENHTISYKTNNYEDSSVENPNQQTSDSSSLQTDEDKKKDDSDATSIGETSPTTETTSVEETVTIEDTESVEETESVEETPSTSTEETSSTGKKTYVDRIASILNPLINGEKKSTEKKSSEKKSSEKKSSDEQSSSDEQNSSDDQNSFDDQKLFEDIDNLINGIKSRYQEFSAKIKSPEFQNKCKSYMNTAKEMIEERRNCAMSFISRNLNALGIDKIFEDEFGGYALLGKMMLTKVFIDNMFIPDFLRNSSTIILTIVYFLIMMFIVGSYLDINQDTKTERRNTNESKLFNRTQPPM

SEQ ID NO: 369-370 - skipped (2 entries)

SEQ ID NO: 371 - protein, 327 aa, unidentified
  source 1..327: note = Plasmodium
MKKGNNGNFSMARNCECKKIDNDDMVSSTRYSNKFGEKFNLISCTKLFALGMLFLICKNCDNSTQTTSAYQEYQYNGLILGKSRILSELDQNKDQNLGYKTQSYEDSSTENASTSYMSTLKTDEESNTAGEDRKEDNNDAAFIGKKSTEKKPHIDRISSTLNPESTDRRKSSDEQKSSNDKNIFKDVDDLINGIKSRYQDITNKIKSPEFQNECKGYINVAKEMIEDHRNHAMRFVSRNLNSLGIDQIFNDEHGGYAFLAKIMLTKVFVDNLFVPNFLKNNSTILSTIVYFLIISFIVSNYLDATQNNERERKNLNKSNIFCRAKPM

SEQ ID NO: 372-373 - skipped (2 entries)

SEQ ID NO: 374 - protein, 406 aa, unidentified
  source 1..406: note = Plasmodium
MKKETKVDNGVIVPPTRHSQNWGKKLNLISCTKIFALIVLFMICQNGDNSTQSTSSYQEYQHNGLVLGKSRILSELDKTETHATDYKTQSNEDSSVEDPNSPTIITELASDSSKLKTDEESENEDTDKKKDDNDSTSIGETSSAGETTSAEEKLYTDIIDSILSSVVIDGKKIFEEKKESDGKNIFEKKPYDGKKLFEEKKPSDEKKTPGEKKSSNEKKTPGEKKSSNEKKSSSEQKTSSEQKPSSEQKPSSEQKPYDQKSFADDLDSIVNGIKSCYQEVANKVKSPEFQSACKEYVNTAKELFDEHKSSALNFVSSSMQNLGINQIFDDENFGGFATLGKLAVSKVMVDNLFIPDYLKNQSPIILTVVYFLILFMTFGTIYDILPNNRRRGNSNAPQTNYKPQPL

SEQ ID NO: 375-376 - skipped (2 entries)

SEQ ID NO: 377 - protein, 175 aa, unidentified
  source 1..175: note = Plasmodium yoelii
MKVPKVSALFLVFLLSVSFLPSSSLCDPNAANTKIPNIPNPTANAQSPTVSGNSIEQTAEEPNAATKVNNVPVPEKKNDAPVENKDIEKVNDQINAKSKTKPLLISAISAVVAIAIASALGYGIYTKKNSNTPNEDEPSIPSTDDDTGVSEESNEQPIPEELPIPETTENTETPQ

SEQ ID NO: 378-379 - skipped (2 entries)

SEQ ID NO: 380 - protein, 114 aa, unidentified
  source 1..114: note = Plasmodium yoelii
MKLAKALYFVAFLLAIKVLTPGSNNYVEANPPNSDKVANGGDRTLIKKIKDNKVAFISTIAATVALAIATTLGVMHYKKNAKENDSDDDIKIIPLSYPHKDPGKPINYEGNPFA

SEQ ID NO: 381 - skipped

SEQ ID NO: 382 - protein, 381 aa, unidentified
  source 1..381: note = Plasmodium falciparum
MGGAAARLGAVILFVVIVGLHGVRGQEYQCYGSSSNTRVLNELNYDNAGTNLYNELEMNYYGKQENWYSLKKNSRSLGENDDGNNEDNEKLRKPKHKKLKQPADGNPDPNANPNVDPNANPNVDPNANPNVDPNANPNANPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSS

SEQ ID NO: 383 - protein, 78 aa, unidentified
  source 1..78: note = Plasmodium falciparum
LKKIKNSISTEWSPCSVTCGNGIQVRIKPGSANKPKDELDYENDIEKKICKMEKCSSVFNVVNSSIGLIMVLSFLFLN

SEQ ID NO: 384 - protein, 66 aa, unidentified
  source 1..66: note = Plasmodium falciparum
DDGNNEDNEKLRKPKHKKLKQPADGNPDPNANPNVDPNANPNVDPNANPNVDPNANPNANPNANPA

SEQ ID NO: 385 - protein, 16 aa, unidentified
  source 1..16: note = Plasmodium falciparum
CRKPKHKKLKQPADGN

SEQ ID NO: 386 - protein, 16 aa, unidentified
  source 1..16: note = Plasmodium falciparum
CHKKLKQPADGNPDPN

SEQ ID NO: 387 - protein, 20 aa, unidentified
  source 1..20: note = Plasmodium falciparum
CKQPADGNPDPNANPNVDPN

SEQ ID NO: 388 - protein, 16 aa, unidentified
  source 1..16: note = Plasmodium falciparum
CPADGNPDPNANPNVD

SEQ ID NO: 389 - protein, 16 aa, unidentified
  source 1..16: note = Plasmodium falciparum
CNPDPNANPNVDPNAN

SEQ ID NO: 390 - protein, 16 aa, unidentified
  source 1..16: note = Plasmodium falciparum
CNANPNVDPNANPNVD

SEQ ID NO: 391 - protein, 16 aa, unidentified
  source 1..16: note = Plasmodium falciparum
CNVDPNANPNVDPNAN

SEQ ID NO: 392 - protein, 16 aa, unidentified
  source 1..16: note = Plasmodium falciparum
CNVDPNANPNANPNAN

SEQ ID NO: 393 - protein, 16 aa, unidentified
  source 1..16: note = Plasmodium falciparum
CNANPNANPNANPNAN

SEQ ID NO: 394 - protein, 16 aa, unidentified
  source 1..16: note = Plasmodium falciparum
CNVDPNANPNVDPNAN

SEQ ID NO: 395 - protein, 397 aa, synthetic construct; identical to SEQ ID NO: 1

SEQ ID NO: 396 - protein, 9 aa, synthetic construct
SSFLFVEAL

SEQ ID NO: 397 - protein, 10 aa, synthetic construct
YSLKKNSRSL

SEQ ID NO: 398 - protein, 9 aa, synthetic construct
TNLYNELEM

SEQ ID NO: 399 - protein, 9 aa, synthetic construct
SANKPKDEL

SEQ ID NO: 400 - protein, 9 aa, synthetic construct
YLNKIQNSL

SEQ ID NO: 401 - protein, 14 aa, synthetic construct
IQVRIKPGSANKPK

SEQ ID NO: 402 - protein, 14 aa, synthetic construct
NVDENANANSAVKN

SEQ ID NO: 403 - protein, 14 aa, synthetic construct
NANPNANPNANPNV

SEQ ID NO: 404 - protein, 13 aa, synthetic construct
VDENANANSAVKN

SEQ ID NO: 405 - protein, 13 aa, synthetic construct
LFQEYQCYGSSSN

SEQ ID NO: 406 - DNA, 72 bp, unidentified
  source 1..72: mol_type = other DNA; note = Plasmodium falciparum
aacgccaatcctaacgtggaccccaacgctaaccccaatgtggacccaaatgccaatccaaatgtcgatccc

SEQ ID NO: 407 - protein, 24 aa, unidentified
  source 1..24: note = Plasmodium falciparum
NANPNVDPNANPNVDPNANPNVDP

SEQ ID NO: 408 - RNA, 72 nt, unidentified
  source 1..72: mol_type = other RNA; note = Plasmodium falciparum
aacgccaatcctaacgtggaccccaacgctaaccccaatgtggacccaaatgccaatccaaatgtcgatccc

SEQ ID NO: 409 - DNA, 60 bp, unidentified
  source 1..60: mol_type = other DNA; note = Plasmodium falciparum
ttcaacgtggtcaacagcagcatcggcctgattatggtgctgtccttcctgttcctgaac

SEQ ID NO: 410-411 - skipped (2 entries)

SEQ ID NO: 412 - DNA, 15 bp, unidentified
  source 1..15: mol_type = other DNA; note = Plasmodium falciparum
aagctgaagcagccc

SEQ ID NO: 413 - protein, 5 aa, synthetic construct
KLKQP

SEQ ID NO: 414 - RNA, 15 nt, unidentified
  source 1..15: mol_type = other RNA; note = Plasmodium falciparum
aagctgaagcagccc

SEQ ID NO: 415 - RNA, 47 nt, synthetic construct
  source 1..47: mol_type = other RNA
aactagtattcttctggtccccacagactcagagagaacccgccacc

SEQ ID NO: 416 - RNA, 278 nt, synthetic construct
  source 1..278: mol_type = other RNA
ctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacacc

SEQ ID NO: 417 - RNA, 110 nt, synthetic construct
  source 1..110: mol_type = other RNA
aaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 418 - DNA, 2379 bp, synthetic construct
  source 1..2379: mol_type = other DNA
atggtggacttcaacgacctgagcgtggaactgaagaaaaccgagctgatcaaagaggacctgcggaacctgagccacatcatcaacaacgagttcagctacttctgccagaacgagaacaagaacgtgtccttcaacaacaacatcagcagctactacaacgacgacatcttcagcaagagcaccctgaacaacctgtacaccagctggaagctggaagatttcagccacttcgacttcagcagcatcctggacatcctgaagcggaaccagtacgtgatgtgcagcatctactttctgctgatcttcagctgcatctattttctcaccctgctgctctacaccaagtgcatcaagaccacactgaagaagtggttctgccggtactgctccgagaacatcaatgagaacaacagcaaccacaacgagcagcggaccgtgctccagaacgtgatcaacaagagctgctacttcatcacctacagcagcattatctgcctgctgctgtttctgctgctgagcggcatcacctatatgcactacttcattaagaccaagaagggcatccactccaacatctgcaacatctatacccggctggacaagttcctgctgaacaagtgtctggaccccaagaaggtggacacctcctgttacagcgccgagcacattctgaacgacctgtccagcatccttgaggagtacaagaaagtcaaacagcaggccaaggacgacacactgctggacgagaacaccccatttccactgctcgaacggtacatcaccacctttaacaagctgaacgtgctgaagaacaacattaacaagaacaatacgaccctggaaaacgagtacttccacacataccccgctctgaagggaatcagcgagacactgaccaccatcatcagcgagggcaacaagaacttcggcaacgcccggaacgtcatcaaagaagtgaagtctaccatcaagtacagcttccacaccgtggacgagacaatccgcaacgtgttcaaggacagcgtgcccaagatcaccggcctgattacacaggccggcaagagcatcaagggcattaacaacaagtacaagatcaaagaacgcatccccaagtacaccaacatcatcctgctgacgaatatcatcctcctgctgcctccattcctgatcctgctgggcatcatcatcttcatgatcttcatcctgatgggctacatccagaaaaacaacaacttcttcatcaagctgttcggccacttcagcgcctacttcggcctgctgaccatcattatcctgagcttcggcatcctgtttctgagcacctctgtgatcggcggcacctcctgcatcctgagcgagagaatcctgaagaacgagctgcgcttcgacatcctcaacaataccctgatcgactactgtatcaagaacgagagcgcccctctgatcgacgacgatatcaccaccagcttcgtggccaagattaactccttcgacaccggccacatcgaccacaacatcaacgagtacgagaagcacttcacgatcctgaaagagagcttcttccacaagagcctgaagttcatggactacatctggatcgtcatcatgaagcgggaaaacaataccttcctgaaccgcatcagaaccgagcaagtgaagaagtccctgctgatcaccgggatcatcaacgaaaacatcaaatacgagaacatggaagccatcggcatccggtcctacctgaccactctgaacaagatcatcttccccgagaacaacgggaagatctgcttcaacgatatcatctgcgagaaagaaaacaacacctacaatatcaccgagaacagcaagaccaccgaccagaagtaccggaacatccgcgacggcatggacgagcacctgagaaacgatctggacgccatcgtgcagctgttcgtgtacaaggcccggattctcaaagagaacatcttcgacattaacgacctggacagcaacgagaagaacaagattggctggagcgagtacacaccccggaatatcaacggcacccagaagaagtctattatcaacaccttcctggtcaacgtgatcgagtccatcaatttcagcgagattatcaatttcttcgacaagatgcgggaccagttcaacgtcctgaaggacctgattctgctgaaaatcgacaccctgaccgagaatacgaagtgcaacaagctggtcaaagaactgatcaatgtgcggaaggactactgcaacaacgtggtgctgaacctgtccacactgagcgtgtacctgatcattttctccatcaccagctttctgctctggtatctctttctgttcctgtggttttactataacatcaagcccagctgatga

SEQ ID NO: 419 - protein, 791 aa, synthetic construct; identical to SEQ ID NO: 249

SEQ ID NO: 420 - RNA, 2379 nt, synthetic construct
  source 1..2379: mol_type = other RNA
atggtggacttcaacgacctgagcgtggaactgaagaaaaccgagctgatcaaagaggacctgcggaacctgagccacatcatcaacaacgagttcagctacttctgccagaacgagaacaagaacgtgtccttcaacaacaacatcagcagctactacaacgacgacatcttcagcaagagcaccctgaacaacctgtacaccagctggaagctggaagatttcagccacttcgacttcagcagcatcctggacatcctgaagcggaaccagtacgtgatgtgcagcatctactttctgctgatcttcagctgcatctattttctcaccctgctgctctacaccaagtgcatcaagaccacactgaagaagtggttctgccggtactgctccgagaacatcaatgagaacaacagcaaccacaacgagcagcggaccgtgctccagaacgtgatcaacaagagctgctacttcatcacctacagcagcattatctgcctgctgctgtttctgctgctgagcggcatcacctatatgcactacttcattaagaccaagaagggcatccactccaacatctgcaacatctatacccggctggacaagttcctgctgaacaagtgtctggaccccaagaaggtggacacctcctgttacagcgccgagcacattctgaacgacctgtccagcatccttgaggagtacaagaaagtcaaacagcaggccaaggacgacacactgctggacgagaacaccccatttccactgctcgaacggtacatcaccacctttaacaagctgaacgtgctgaagaacaacattaacaagaacaatacgaccctggaaaacgagtacttccacacataccccgctctgaagggaatcagcgagacactgaccaccatcatcagcgagggcaacaagaacttcggcaacgcccggaacgtcatcaaagaagtgaagtctaccatcaagtacagcttccacaccgtggacgagacaatccgcaacgtgttcaaggacagcgtgcccaagatcaccggcctgattacacaggccggcaagagcatcaagggcattaacaacaagtacaagatcaaagaacgcatccccaagtacaccaacatcatcctgctgacgaatatcatcctcctgctgcctccattcctgatcctgctgggcatcatcatcttcatgatcttcatcctgatgggctacatccagaaaaacaacaacttcttcatcaagctgttcggccacttcagcgcctacttcggcctgctgaccatcattatcctgagcttcggcatcctgtttctgagcacctctgtgatcggcggcacctcctgcatcctgagcgagagaatcctgaagaacgagctgcgcttcgacatcctcaacaataccctgatcgactactgtatcaagaacgagagcgcccctctgatcgacgacgatatcaccaccagcttcgtggccaagattaactccttcgacaccggccacatcgaccacaacatcaacgagtacgagaagcacttcacgatcctgaaagagagcttcttccacaagagcctgaagttcatggactacatctggatcgtcatcatgaagcgggaaaacaataccttcctgaaccgcatcagaaccgagcaagtgaagaagtccctgctgatcaccgggatcatcaacgaaaacatcaaatacgagaacatggaagccatcggcatccggtcctacctgaccactctgaacaagatcatcttccccgagaacaacgggaagatctgcttcaacgatatcatctgcgagaaagaaaacaacacctacaatatcaccgagaacagcaagaccaccgaccagaagtaccggaacatccgcgacggcatggacgagcacctgagaaacgatctggacgccatcgtgcagctgttcgtgtacaaggcccggattctcaaagagaacatcttcgacattaacgacctggacagcaacgagaagaacaagattggctggagcgagtacacaccccggaatatcaacggcacccagaagaagtctattatcaacaccttcctggtcaacgtgatcgagtccatcaatttcagcgagattatcaatttcttcgacaagatgcgggaccagttcaacgtcctgaaggacctgattctgctgaaaatcgacaccctgaccgagaatacgaagtgcaacaagctggtcaaagaactgatcaatgtgcggaaggactactgcaacaacgtggtgctgaacctgtccacactgagcgtgtacctgatcattttctccatcaccagctttctgctctggtatctctttctgttcctgtggttttactataacatcaagcccagctgatga

SEQ ID NO: 421 - DNA, 2379 bp, synthetic construct
  source 1..2379: mol_type = other DNA
atggttgattttaatgatttgtctgttgaattgaagaaaacagaattgataaaagaagatttgagaaatttgtctcacataataaataatgaattttcttacttttgccaaaatgaaaataaaaatgtttcttttaataataatatttcttcttactacaatgatgatattttctcaaaatcaacattgaataatttgtacacatcttggaaattggaagatttctctcactttgatttctcatcaattttggatattttgaaaagaaatcagtacgtcatgtgctcaatttactttttgttgattttctcttgcatttactttttgacattgttgttgtacacaaaatgcattaaaacaacattgaagaaatggttttgcagatactgctctgaaaatataaatgaaaataattcaaatcacaatgaacaaagaacagttttacaaaatgtcataaataaatcttgctactttattacatactcatcaataatatgccttcttctttttcttcttctttctggaataacatacatgcactactttataaagacaaagaaaggaatacactcaaatatttgcaatatttacacaagacttgataaatttttgttgaataaatgcttggatccaaagaaagttgatacatcttgctactctgctgaacacattttgaatgatttgtcatcaattttggaagaatacaaaaaggtgaagcaacaagcaaaagatgatacacttcttgatgaaaatacaccttttccacttcttgaaagatacataacaacatttaataaattgaatgttttgaaaaataatataaataaaaataatacaacacttgaaaatgaatactttcacacataccctgctttgaaaggaatttctgaaacattgacaacaataatatctgaaggaaataaaaattttggaaatgcaagaaatgtcattaaagaagtcaaatcaacaataaaatcatcatttcacacagttgatgaaacaataagaaatgtttttaaagattctgttccaaaaataacaggattaataacacaagctggaaaatcaataaaaggaataaataataaatacaaaattaaagaaagaattccaaaatacacaaatattattttgttgacaaatattattttgttgttgcctcctttcttgattttgttgggaattattatttttatgatttttattttgatgggatacattcagaaaaataataatttctttataaaattgtttggacacttttctgcttactttggattgttgacaataataatattgtcttttggaatattgtttttgtcaacatcagtcattggaggaacatcatgcattttatcagaaagaattttaaagaatgaattaagatttgatattttaaataatacattaattgattactgcattaaaaatgaatctgctcctttaattgatgatgatattacaacatcttttgttgcaaaaattaattcttttgatacaggacacattgatcacaatattaatgaatacgaaaaacactttacaattttaaaagaatctttctttcacaaatctttaaaatttatggattacatttggattgtcattatgaaaagagaaaataatacatttttaaatagaattagaacagaacaagtcaagaaatctttattaataacaggaataataaatgaaaatataaaatatgaaaatatggaagcaataggaataagatcttacttaacaacattaaataaaattatttttcctgaaaataatggaaaaatttgctttaatgatattatttgtgaaaaagaaaataatacatacaatattacagaaaattcaaaaacaacagatcagaaatacagaaatataagagatggaatggatgaacacttaagaaatgatttagatgcaattgttcaattatttgtttacaaagcaagaattttaaaagaaaatatttttgatattaatgatttagattcaaatgagaaaaataaaattggatggtcagaatacacaccaagaaatattaatggaacacaaaagaaatcaattattaatacatttttagtcaatgtcattgaatcaattaatttttcagaaattattaatttctttgataaaatgagagatcaatttaatgttttgaaagatttgattttgttgaaaattgatacattgacagaaaatacaaaatgcaataaattggtcaaagaattaattaatgtcagaaaagattactgcaataatgtcgtcttgaatttgtcaacattatcagtttacttaattattttctcaattacatcatttttattatggtacttatttttatttttatggttttactacaatattaaaccttcttgatga

SEQ ID NO: 422 - protein, 791 aa, synthetic construct; identical to SEQ ID NO: 249

SEQ ID NO: 423 - RNA, 2379 nt, synthetic construct
  source 1..2379: mol_type = other RNA
atggttgattttaatgatttgtctgttgaattgaagaaaacagaattgataaaagaagatttgagaaatttgtctcacataataaataatgaattttcttacttttgccaaaatgaaaataaaaatgtttcttttaataataatatttcttcttactacaatgatgatattttctcaaaatcaacattgaataatttgtacacatcttggaaattggaagatttctctcactttgatttctcatcaattttggatattttgaaaagaaatcagtacgtcatgtgctcaatttactttttgttgattttctcttgcatttactttttgacattgttgttgtacacaaaatgcattaaaacaacattgaagaaatggttttgcagatactgctctgaaaatataaatgaaaataattcaaatcacaatgaacaaagaacagttttacaaaatgtcataaataaatcttgctactttattacatactcatcaataatatgccttcttctttttcttcttctttctggaataacatacatgcactactttataaagacaaagaaaggaatacactcaaatatttgcaatatttacacaagacttgataaatttttgttgaataaatgcttggatccaaagaaagttgatacatcttgctactctgctgaacacattttgaatgatttgtcatcaattttggaagaatacaaaaaggtgaagcaacaagcaaaagatgatacacttcttgatgaaaatacaccttttccacttcttgaaagatacataacaacatttaataaattgaatgttttgaaaaataatataaataaaaataatacaacacttgaaaatgaatactttcacacataccctgctttgaaaggaatttctgaaacattgacaacaataatatctgaaggaaataaaaattttggaaatgcaagaaatgtcattaaagaagtcaaatcaacaataaaatcatcatttcacacagttgatgaaacaataagaaatgtttttaaagattctgttccaaaaataacaggattaataacacaagctggaaaatcaataaaaggaataaataataaatacaaaattaaagaaagaattccaaaatacacaaatattattttgttgacaaatattattttgttgttgcctcctttcttgattttgttgggaattattatttttatgatttttattttgatgggatacattcagaaaaataataatttctttataaaattgtttggacacttttctgcttactttggattgttgacaataataatattgtcttttggaatattgtttttgtcaacatcagtcattggaggaacatcatgcattttatcagaaagaattttaaagaatgaattaagatttgatattttaaataatacattaattgattactgcattaaaaatgaatctgctcctttaattgatgatgatattacaacatcttttgttgcaaaaattaattcttttgatacaggacacattgatcacaatattaatgaatacgaaaaacactttacaattttaaaagaatctttctttcacaaatctttaaaatttatggattacatttggattgtcattatgaaaagagaaaataatacatttttaaatagaattagaacagaacaagtcaagaaatctttattaataacaggaataataaatgaaaatataaaatatgaaaatatggaagcaataggaataagatcttacttaacaacattaaataaaattatttttcctgaaaataatggaaaaatttgctttaatgatattatttgtgaaaaagaaaataatacatacaatattacagaaaattcaaaaacaacagatcagaaatacagaaatataagagatggaatggatgaacacttaagaaatgatttagatgcaattgttcaattatttgtttacaaagcaagaattttaaaagaaaatatttttgatattaatgatttagattcaaatgagaaaaataaaattggatggtcagaatacacaccaagaaatattaatggaacacaaaagaaatcaattattaatacatttttagtcaatgtcattgaatcaattaatttttcagaaattattaatttctttgataaaatgagagatcaatttaatgttttgaaagatttgattttgttgaaaattgatacattgacagaaaatacaaaatgcaataaattggtcaaagaattaattaatgtcagaaaagattactgcaataatgtcgtcttgaatttgtcaacattatcagtttacttaattattttctcaattacatcatttttattatggtacttatttttatttttatggttttactacaatattaaaccttcttgatga

SEQ ID NO: 424 - RNA, 12 nt, synthetic construct
  source 1..12: mol_type = other RNA
aaaaaaattttt

SEQ ID NO: 425 - protein, 9 aa, synthetic construct
SPSYVYHQF

SEQ ID NO: 426 - protein, 4 aa, synthetic construct
AAKQ

SEQ ID NO: 427 - RNA, 53 nt, synthetic construct
  source 1..53: mol_type = other RNA
agaataaactagtattcttctggtccccacagactcagagagaacccgccacc

SEQ ID NO: 428 - RNA, 110 nt, synthetic construct; identical to SEQ ID NO: 417
  source 1..110: mol_type = other RNA

SEQ ID NO: 429 - RNA, 278 nt, synthetic construct; identical to SEQ ID NO: 416
  source 1..278: mol_type = other RNA

SEQ ID NO: 430-431 - skipped (2 entries)

SEQ ID NO: 432 - protein, 287 aa, synthetic construct
MMRKLAILSVSSFLFVEAEDNEKLRKPKHKKLKQPADGNPDPNANPNVDPNANPNVDPNANPNVDPNANPNANPNANPNANPNANPNANPNANPNVDPNANPNVDPNANPNVDPNANPNANPNANPNANPNANPNANPNANPNVDPNANPNVDPNANPNVDPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVFNVVNSSIGLIMVLSFLFLN

SEQ ID NO: 433 - protein, 267 aa, synthetic construct
MMRKLAILSVSSFLFVEAEDNEKLRKPKHKKLKQPADGNPDPNANPNVDPNANPNVDPNANPNVDPNANPNANPNANPNANPNANPNANPNANPNVDPNANPNVDPNANPNVDPNANPNANPNANPNANPNANPNANPNANPNVDPNANPNVDPNANPNVDPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSV

SEQ ID NO: 434 - protein, 287 aa, synthetic construct
MMRKLAILSVSSFLFVEAEDNEKLRKPKHKKLKQPADGNPDPNANPNVDPNANPNVDPNANPNVDPNANPNANPNANPNANPNANPNANPNANPNVDPNANPNVDPNANPNVDPNANPNANPNANPNANPNANPNANPNANPNVDPNANPNVDPNANPNVDPNKNNQGNGQGHNMPNDPNRNVDENANANSAVKNNNNEEPSDKHIKEYLNKIQNSLSTEWSPCSVNCGNGIQVRIKPGSANKPKDELDYANDIEKKICKMEKCSSVFNVVNSSIGLIMVLSFLFLN

SEQ ID NO: 435 - protein, 268 aa, synthetic construct
MGGAAARLGAVILFVVIVGLHGVRGADGNPDPADGNPDPNANPNVDPNANPNVDPNANPNVDPNANPNANPNANPNANPNANPNANPNAADGNPDPADGNPDPNANPNVDPNANPNVDPNANPNVDPNANPNANPNANPNANPNANPNANPNAADGNPDPADGNPDPNANPNVDPNANPNVDPNANPNVDPNANPNANPNANPNANPNANPNANPNAGGSGGGGSGGGLIAGAVGGSLLAALVICGIVYWMRRHTQKAPKRIRLPHIR

SEQ ID NO: 436 - protein, 303 aa, synthetic construct
MGGAAARLGAVILFVVIVGLHGVRGKLKQPADGNPDPADGNPDPNANPNVDPNANPNVDPNANPNVDPNANPNANPNANPNANPNANPNANPNAGGSGGGGSGGKLKQPADGNPDPADGNPDPNANPNVDPNANPNVDPNANPNVDPNANPNANPNANPNANPNANPNANPNAGGSGGGGSGGKLKQPADGNPDPADGNPDPNANPNVDPNANPNVDPNANPNVDPNANPNANPNANPNANPNANPNANPNAGGSGGGGSGGGLIAGAVGGSLLAALVICGIVYWMRRHTQKAPKRIRLPHIR

SEQ ID NO: 437 - protein, 24 aa, synthetic construct
NANPNANPNANPNANPNANPNANP

SEQ ID NO: 438 - protein, 9 aa, synthetic construct
TAPDNLGYA

SEQ ID NO: 439 - protein, 104 aa, synthetic construct
NKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSS

SEQ ID NO: 440 - protein, 6 aa, synthetic construct
HHHHHH

SEQ ID NO: 441 - DNA, 867 bp, synthetic construct
  source 1..867: mol_type = other DNA
atgatgcggaagctggccatcctgagcgtgtccagcttcctgtttgtggaagccgaggacaacgagaagctgcggaagcccaagcacaagaagctgaagcagcccgccgacggcaaccccgatcctaacgctaatcccaacgtggaccccaacgccaatcctaatgtcgacccaaacgcaaacccaaacgtcgaccctaatgctaaccccaatgcaaatccgaacgctaacccgaacgctaatccaaatgccaatcctaacgcaaaccccaatgctaaccctaacgttgaccccaatgctaatccaaatgttgatccgaacgccaatccgaacgttgaccctaacgctaacccaaacgctaaccccaacgcaaatcccaatgcaaaccccaatgccaatccaaacgccaatccaaatgcaaatccgaatgtggaccccaatgcaaacccgaatgtcgatcccaacgctaaccctaatgtggaccctaacaagaacaatcaaggcaacggccagggccacaacatgcctaacgaccccaacagaaacgtggacgagaacgcaaatgctaacagcgccgtgaagaacaacaacaacgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggacgagctggactacgccaacgacatcgagaagaaaatctgcaagatggaaaagtgcagcagcgtgttcaacgtggtcaacagcagcatcggcctgattatggtgctgtccttcctgttcctgaactgataa

SEQ ID NO: 442 - RNA, 867 nt, synthetic construct
  source 1..867: mol_type = other RNA
atgatgcggaagctggccatcctgagcgtgtccagcttcctgtttgtggaagccgaggacaacgagaagctgcggaagcccaagcacaagaagctgaagcagcccgccgacggcaaccccgatcctaacgctaatcccaacgtggaccccaacgccaatcctaatgtcgacccaaacgcaaacccaaacgtcgaccctaatgctaaccccaatgcaaatccgaacgctaacccgaacgctaatccaaatgccaatcctaacgcaaaccccaatgctaaccctaacgttgaccccaatgctaatccaaatgttgatccgaacgccaatccgaacgttgaccctaacgctaacccaaacgctaaccccaacgcaaatcccaatgcaaaccccaatgccaatccaaacgccaatccaaatgcaaatccgaatgtggaccccaatgcaaacccgaatgtcgatcccaacgctaaccctaatgtggaccctaacaagaacaatcaaggcaacggccagggccacaacatgcctaacgaccccaacagaaacgtggacgagaacgcaaatgctaacagcgccgtgaagaacaacaacaacgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggacgagctggactacgccaacgacatcgagaagaaaatctgcaagatggaaaagtgcagcagcgtgttcaacgtggtcaacagcagcatcggcctgattatggtgctgtccttcctgttcctgaactgataa

SEQ ID NO: 443 - DNA, 807 bp, synthetic construct
  source 1..807: mol_type = other DNA
atgatgcggaagctggccatcctgagcgtgtccagcttcctgtttgtggaagccgaggacaacgagaagctgcggaagcccaagcacaagaagctgaagcagcccgccgacggcaaccccgatcctaacgctaatcccaacgtggaccccaacgccaatcctaatgtcgacccaaacgcaaacccaaacgtcgaccctaatgctaaccccaatgcaaatccgaacgctaacccgaacgctaatccaaatgccaatcctaacgcaaaccccaatgctaaccctaacgttgaccccaatgctaatccaaatgttgatccgaacgccaatccgaacgttgaccctaacgctaacccaaacgctaaccccaacgcaaatcccaatgcaaaccccaatgccaatccaaacgccaatccaaatgcaaatccgaatgtggaccccaatgcaaacccgaatgtcgatcccaacgctaaccctaatgtggaccctaacaagaacaatcaaggcaacggccagggccacaacatgcctaacgaccccaacagaaacgtggacgagaacgcaaatgctaacagcgccgtgaagaacaacaacaacgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggacgagctggactacgccaacgacatcgagaagaaaatctgcaagatggaaaagtgcagcagcgtgtgataa

SEQ ID NO: 444 - RNA, 807 nt, synthetic construct
  source 1..807: mol_type = other RNA
atgatgcggaagctggccatcctgagcgtgtccagcttcctgtttgtggaagccgaggacaacgagaagctgcggaagcccaagcacaagaagctgaagcagcccgccgacggcaaccccgatcctaacgctaatcccaacgtggaccccaacgccaatcctaatgtcgacccaaacgcaaacccaaacgtcgaccctaatgctaaccccaatgcaaatccgaacgctaacccgaacgctaatccaaatgccaatcctaacgcaaaccccaatgctaaccctaacgttgaccccaatgctaatccaaatgttgatccgaacgccaatccgaacgttgaccctaacgctaacccaaacgctaaccccaacgcaaatcccaatgcaaaccccaatgccaatccaaacgccaatccaaatgcaaatccgaatgtggaccccaatgcaaacccgaatgtcgatcccaacgctaaccctaatgtggaccctaacaagaacaatcaaggcaacggccagggccacaacatgcctaacgaccccaacagaaacgtggacgagaacgcaaatgctaacagcgccgtgaagaacaacaacaacgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggacgagctggactacgccaacgacatcgagaagaaaatctgcaagatggaaaagtgcagcagcgtgtgataa

SEQ ID NO: 445 - DNA, 867 bp, synthetic construct
  source 1..867: mol_type = other DNA
atgatgcggaagctggccatcctgagcgtgtccagcttcctgtttgtggaagccgaggacaacgagaagctgcggaagcccaagcacaagaagctgaagcagcccgccgacggcaaccccgatcctaacgctaatcccaacgtggaccccaacgccaatcctaatgtcgacccaaacgcaaacccaaacgtcgaccctaatgctaaccccaatgcaaatccgaacgctaacccgaacgctaatccaaatgccaatcctaacgcaaaccccaatgctaaccctaacgttgaccccaatgctaatccaaatgttgatccgaacgccaatccgaacgttgaccctaacgctaacccaaacgctaaccccaacgcaaatcccaatgcaaaccccaatgccaatccaaacgccaatccaaatgcaaatccgaatgtggaccccaatgcaaacccgaatgtcgatcccaacgctaaccctaatgtggaccctaacaagaacaatcaaggcaacggccagggccacaacatgcctaacgaccccaacagaaacgtggacgagaacgcaaatgctaacagcgccgtgaagaacaacaacaacgaggaacccagcgacaagcacatcaaagagtacctgaacaagatccagaacagcctgagcaccgagtggtccccatgctccgtgaattgcggcaacggaatccaagtgcggatcaagcctggcagcgccaacaagcctaaggacgagctggactacgccaacgacatcgagaagaaaatctgcaagatggaaaagtgcagcagcgtgttcaacgtggtcaacagcagcatcggcctgattatggtgctgtccttcctgttcctgaactgataa

SEQ ID NO: 446 - RNA, 867 nt, synthetic construct
  source 1..867: mol_type = other RNA
atgatgcggaagctggccatcctgagcgtgtccagcttcctgtttgtggaagccgaggacaacgagaagctgcggaagcccaagcacaagaagctgaagcagcccgccgacggcaaccccgatcctaacgctaatcccaacgtggaccccaacgccaatcctaatgtcgacccaaacgcaaacccaaacgtcgaccctaatgctaaccccaatgcaaatccgaacgctaacccgaacgctaatccaaatgccaatcctaacgcaaaccccaatgctaaccctaacgttgaccccaatgctaatccaaatgttgatccgaacgccaatccgaacgttgaccctaacgctaacccaaacgctaaccccaacgcaaatcccaatgcaaaccccaatgccaatccaaacgccaatccaaatgcaaatccgaatgtggaccccaatgcaaacccgaatgtcgatcccaacgctaaccctaatgtggaccctaacaagaacaatcaaggcaacggccagggccacaacatgcctaacgaccccaacagaaacgtggacgagaacgcaaatgctaacagcgccgtgaagaacaacaacaacgaggaacccagcgacaagcacatcaaagagtacctgaacaagatccagaacagcctgagcaccgagtggtccccatgctccgtgaattgcggcaacggaatccaagtgcggatcaagcctggcagcgccaacaagcctaaggacgagctggactacgccaacgacatcgagaagaaaatctgcaagatggaaaagtgcagcagcgtgttcaacgtggtcaacagcagcatcggcctgattatggtgctgtccttcctgttcctgaactgataa

SEQ ID NO: 447 - DNA, 810 bp, synthetic construct
  source 1..810: mol_type = other DNA
atgggcggagctgctgctagactgggagccgtgatcctgttcgtggttatcgtgggactgcatggcgtcagaggcgccgatggaaatcctgatcctgccgacggcaaccccgatcctaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatccgaacgccgccgacgggaatccagatccagctgatggaaacccagatccaaatgctaatcccaacgttgacccgaatgcaaatcctaatgttgacccaaacgcaaacccaaacgtcgaccccaacgcaaatccaaacgctaaccctaatgcaaacccaaatgcaaatcccaacgccaatcctaatgctaaccccaacgccgccgatggcaaccctgatccagcagatgggaatcctgatccgaacgctaatcctaatgtcgaccctaatgctaatccgaatgtggacccgaacgctaatccaaacgttgaccctaacgccaatccgaatgccaatcctaatgcaaatcctaacgctaatcccaacgcaaacccgaacgctaatcctaacgcaggcggatctggtggcggaggttctggcggaggacttattgctggtgccgttggcggatctctgctggccgctctggttatctgtggcatcgtgtactggatgcggcggcacacacagaaggcccctaagagaatcagactgccccacatcagatgataa

SEQ ID NO: 448 - RNA, 810 nt, synthetic construct
  source 1..810: mol_type = other RNA
atgggcggagctgctgctagactgggagccgtgatcctgttcgtggttatcgtgggactgcatggcgtcagaggcgccgatggaaatcctgatcctgccgacggcaaccccgatcctaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatccgaacgccgccgacgggaatccagatccagctgatggaaacccagatccaaatgctaatcccaacgttgacccgaatgcaaatcctaatgttgacccaaacgcaaacccaaacgtcgaccccaacgcaaatccaaacgctaaccctaatgcaaacccaaatgcaaatcccaacgccaatcctaatgctaaccccaacgccgccgatggcaaccctgatccagcagatgggaatcctgatccgaacgctaatcctaatgtcgaccctaatgctaatccgaatgtggacccgaacgctaatccaaacgttgaccctaacgccaatccgaatgccaatcctaatgcaaatcctaacgctaatcccaacgcaaacccgaacgctaatcctaacgcaggcggatctggtggcggaggttctggcggaggacttattgctggtgccgttggcggatctctgctggccgctctggttatctgtggcatcgtgtactggatgcggcggcacacacagaaggcccctaagagaatcagactgccccacatcagatgataa

SEQ ID NO: 449 - DNA, 915 bp, synthetic construct
  source 1..915: mol_type = other DNA
atgggcggagctgctgctagactgggagccgtgatcctgttcgtggttatcgtgggactgcatggcgtgcggggaaagctgaaacagcctgccgatggcaatcccgatccagccgacggaaaccccgatcctaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatccgaacgctggcggatctggtggcggaggatctggcggaaaactgaagcaacccgctgacggcaatcctgatcctgctgatgggaaccctgatccaaatgctaatcccaacgttgacccgaatgcaaatcctaatgttgacccaaacgcaaacccaaacgtcgaccccaacgcaaatccaaacgctaaccctaatgcaaacccaaatgcaaatcccaacgccaatcctaatgctaaccccaacgcaggcggatctggcggaggtggaagcggaggaaaactcaaacaaccagcagatggcaacccagatccggcagatggaaatccagatcctaacgctaacccaaatgttgatccgaacgctaacccgaacgtggaccccaatgcaaatcccaacgtcgaccctaacgctaaccctaacgccaatccaaacgccaatccgaacgctaacccaaacgccaatcctaatgctaatcctaatgccggcggaagcggcggtggtggttcaggcggaggacttattgctggcgccgttggaggatctctgctggccgctctggttatctgcggcatcgtgtactggatgcggcggcacacacagaaggcccctaagagaatcagactgccccacatcagatgataa

SEQ ID NO: 450 - RNA, 915 nt, synthetic construct
  source 1..915: mol_type = other RNA
atgggcggagctgctgctagactgggagccgtgatcctgttcgtggttatcgtgggactgcatggcgtgcggggaaagctgaaacagcctgccgatggcaatcccgatccagccgacggaaaccccgatcctaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatccgaacgctggcggatctggtggcggaggatctggcggaaaactgaagcaacccgctgacggcaatcctgatcctgctgatgggaaccctgatccaaatgctaatcccaacgttgacccgaatgcaaatcctaatgttgacccaaacgcaaacccaaacgtcgaccccaacgcaaatccaaacgctaaccctaatgcaaacccaaatgcaaatcccaacgccaatcctaatgctaaccccaacgcaggcggatctggcggaggtggaagcggaggaaaactcaaacaaccagcagatggcaacccagatccggcagatggaaatccagatcctaacgctaacccaaatgttgatccgaacgctaacccgaacgtggaccccaatgcaaatcccaacgtcgaccctaacgctaaccctaacgccaatccaaacgccaatccgaacgctaacccaaacgccaatcctaatgctaatcctaatgccggcggaagcggcggtggtggttcaggcggaggacttattgctggcgccgttggaggatctctgctggccgctctggttatctgcggcatcgtgtactggatgcggcggcacacacagaaggcccctaagagaatcagactgccccacatcagatgataa